(19) United States
(12) Patent Application Publication
(10) Pub. No.: US 2020/0354419 A1
(43) Pub. Date: Nov. 12, 2020

(54) COMPOSITIONS AND METHODS OF USE THEREOF FOR THE TREATMENT OF DUCHENNE MUSCULAR DYSTROPHY

(71) Applicant: HUNTERIAN MEDICINE LLC, Cambridge, MA (US)

(72) Inventor: Vinod Jaskula-Ranga, Cambridge, MA (US)

(21) Appl. No.: 16/761,112

(22) PCT Filed: Nov. 2, 2018
(86) PCT No.: PCT/US18/59074

§ 371 Date: May 1, 2020

Related U.S. Application Data
(60) Provisional application No. 62/581,493, filed on Nov. 3, 2017.

(51) Int. Cl. C07K 14/47; C12N 15/113; A61K 48/00; C12N 15/86

(52) U.S. Cl. CPC C07K 14/4708; C12N 15/113; A61K 48/005; A61K 38/00; C12N 2310/20; C12N 2310/33; C12N 2750/14143; C12N 15/86

(57) ABSTRACT

The present disclosure provides, in part, a method of treating subjects with Duchenne muscular dystrophy (DMD) via gene editing approaches that induce exon deletion(s) to restore the reading frame of the dystrophin gene, thereby restoring dystrophin protein activity. The invention also provides compositions comprising adeno-associated viral vectors, an RNA-guided nucleases, nickases or DNA endonucleases, and guide RNAs for use in the treatment of subjects with DMD, or subjects with other neuromuscular genetic diseases or disorders.

### CROSS-REFERENCE TO RELATED APPLICATIONS

This application claims the benefit of U.S. Provisional Patent Application No. 62/581,493, filed Nov. 3, 2017, the entire contents of which are incorporated by reference herein.

### FIELD OF THE INVENTION

The present disclosure relates to the fields of molecular biology, medicine and genetics. In particular, the present disclosure provides therapeutic compositions and their use in genome editing based methods for the treatment of Duchenne muscular dystrophy and other genetic diseases or disorders.

### BACKGROUND OF THE INVENTION

Duchenne muscular dystrophy (DMD) is an X-linked neuromuscular disorder and is the most frequent lethal heritable childhood disease: DMD affects approximately 1 in 3500 to 5000 boys. DMD is a severe disease, characterized by progressive muscle weakness that leads to DMD patients being confined to wheelchairs by the age of 12, and ultimately death at around age 25.

DMD is caused by a wide variety of mutations in the gene encoding dystrophin at locus Xp21, located on the short arm of the X chromosome. The dystrophin gene product is a large intracellular protein that links the dystroglycan complex at the cell surface with the underlying cytoskeleton. As such, dystrophin protein maintains the integrity of muscle cell membranes during contraction. With 79 exons, the dystrophin gene is one of the largest protein encoding genes in the human genome, and this size makes the dystrophin gene prone to alterations such as deletions and duplications. Frame disrupting alterations to the dystrophin gene result in premature stop codons, and subsequently lead to a truncated protein. Truncated dystrophin proteins cannot provide the structural support necessary to withstand the stress of muscle contraction. This leads to muscle fiber damage and membrane leakage that ultimately manifest as the muscle wasting, breathing complications, and cardiomyopathy that are symptomatic of DMD.

Many different therapeutic strategies for DMD have been explored over the years but existing approved treatments are palliative at best. Beta-2 adrenergic receptor agonists are sometimes used to increase muscle strength, but they do not modify disease progression. A common treatment for DMD is the use of steroids (e.g., prednisone and deflazacort) that have been found to be effective in slowing the course of muscle loss. However, such treatment can lead to side effects such as delayed onset of puberty. Also, rapid withdrawal of corticosteroids can result in life-threatening complications. Additionally, several gene- and cell-based therapies have been developed that aim to deliver functional dystrophin genes or proteins to diseased muscle tissue. However, these approaches have been challenged by the large size of the dystrophin gene, low efficiency, limited persistence of transgene expression, and the host immune response to the vectors used to package the dystrophin gene or protein. As such, no curative treatment for DMD currently exists.

Accordingly, there is a clear, on-going, and urgent need to develop new approaches for the treatment of DMD that provide lifelong restoration of dystrophin expression and activity in the various muscles of the body, in order to improve patient quality of life and to reduce DMD associated morbidity. Recent advances in genome editing technologies, to correct a gene carrying a harmful mutation for example, have sparked enthusiasm and hope for the development of such novel therapeutic strategies for DMD, as well as other genetic diseases, disorders or conditions. Thus, the present disclosure provides genome editing methods, and compositions for use in such methods, for the treatment of DMD and other genetic muscular, neurological, or other diseases, disorders or conditions.

### SUMMARY OF THE INVENTION

The present disclosure presents a genome engineering approach to address the genetic basis of DMD, as well as other genetic diseases, disorders or conditions. The present disclosure provides gene editing methods to create changes to the genome of such patients that can restore the reading frame and the protein activity of mutated genes, thereby effecting permanent corrections to the underlying genetic defect causing the disease, disorder or condition.

Described herein in part is a method of treating a subject (e.g., a human) with Duchenne muscular dystrophy (DMD), comprising administering to the subject a composition comprising an adeno-associated viral vector, an RNA-guided nuclease or nickase or a DNA endonuclease, and at least one dystrophin-targeted guide RNA in amount sufficient to delete a skippable exon encoding the dystrophin gene, wherein the subject with DMD has a mutation causing a frameshift in the dystrophin gene and wherein the deletion of the skippable exon prevents the frameshift. In some embodiments, preventing the frameshift in the dystrophin gene results in partial or complete restoration of dystrophin protein activity.

In some embodiments, the DNA endonuclease is a Cas9 endonuclease or a Cpf1 endonuclease. In some embodiments, the composition comprises two dystrophin-targeted guide RNAs that recognize different DNA sequences.

In some embodiments, the skippable exon is selected from the group consisting of exon 2, exon 8, exon 17, exon 43, exon 44, exon 45, exon 46, exon 50, exon 51, exon 52, exon 53, and exon 55 of the dystrophin gene. In some embodiments, the skippable exon is selected based on the deleted exon or deleted exons that specifically occur in the dystrophin gene of the subject. For example, in some embodiments, the skippable exon is selected from the list in Table 1 according to the indicated dystrophin exon deletions.

The mutation may be a deletion, an insertion, a duplication, or a translocation. In some embodiments, the mutation is a deletion of exons 3-7, 3-19, 3-21, 4-7, 5-7, 6-7, 12-16, 18-33, 18-41, 18-44, 44, 44-47, 44-49, 44-51, 14-43, 19-43, 30-43, 35-43, 36-43, 40-43, 42-43, 45, 45-54, 12-44, 18-44, 46-47, 46-48, 46-49, 46-51, 46-53, 46-55, 21-45, 47-54, 47-56, 51, 51-53, 51-55, 45-50, 47-50, 48-50, 49-50, 50, 52, 52-63, 53, 53-55, 10-52, 45-52, 46-52, 47-52, 48-52, 49-52, 50-52, 45-54, or 48-54 of the dystrophin gene.

Also provided herein is a method of treating a subject (e.g., a human) with Duchenne muscular dystrophy (DMD), comprising administering to the subject a composition comprising an adeno-associated viral vector, an RNA-guided nuclease or nickase or a DNA endonuclease, and at least one dystrophin targeted guide RNA in amount sufficient to restore the reading frame of the dystrophin gene in the subject wherein the dystrophin targeted guide RNA recognizes a target site in the dystrophin gene selected from the sequences identified in Table 2. In some embodiments, one or more target sites recognized by the at least one dystrophin targeted guide RNA occur in an intron of the dystrophin gene.

In some embodiments, the DNA endonuclease is a Cas9 endonuclease or a Cpf1 endonuclease. In some embodiments, the composition comprises two dystrophin-targeted guide RNAs that recognize different target sites.

In some embodiments, restoring the reading frame of the dystrophin gene results in partial or complete restoration of dystrophin protein activity.

The present disclosure also contemplates a method of treating a subject (e.g., a human) with a genetic disease or disorder, comprising a gene editing strategy comprising administering to the subject a composition comprising an adeno-associated viral vector, an RNA-guided nuclease or nickase or a DNA endonuclease, at least one guide RNA, and optionally a donor DNA template in an amount sufficient (i) to delete a skippable exon encoding a mutated gene, wherein such deletion prevents a frameshift of the mutated gene; or (ii) to correct the DNA sequence of the mutated gene via homology directed repair (HDR), wherein the mutated gene is associated with the cause of the disease or disorder; and wherein said method of treatment reduces at least one symptom associated with the disease or disorder. The donor DNA template may comprise a correct DNA sequence (e.g., a wild type or non-mutated sequence) for insertion in the mutated gene via a homology directed repair mechanism.

In some embodiments, the DNA endonuclease is a Cas9 endonuclease or a Cpf1 endonuclease. In some embodiments, the composition comprises two guide RNAs that recognize different target sites. In some embodiments, the guide RNA(s) recognizes a target site in a gene selected from the group consisting of MYH7, TNNT2, TPM1, MYBPC3, PRKAG2, TNNI3, MYL3, TNN, MYL2, ACTC1, CSRP3, TNNC1, MYH6, VCL, MYOZ2, JPH2, PLN, CALR3, NEXN, MYPN, ACTN2, LDB3, TCAP, FLNC, SOD1, C9ORF72, and RYR.

In some embodiments, the genetic disease or disorder can be a muscle-related disease or disorder selected from the group consisting of Hypertrophic cardiomyopathy (HCM or CMH), amyotrophic lateral sclerosis, Becker's muscular dystrophy, central core disease, centronuclear myopathy (including myotubular myopathy), Charcot-Marie-Tooth disease, congenital muscular dystrophy, congenital myasthenic syndrome, Dejerine-Sottas disease, dermatomyositis, Duchenne muscular dystrophy, Emery-Dreifuss muscular dystrophy, facioscapulohumeral muscular dystrophy, Friedreich's ataxia, hyperthyroid myopathy, hypothyroid myopathy, inclusion body myositis, Lambert-Eaton myasthenic syndrome, Limb-girdle muscular dystrophy, mitochondrial myopathy, myasthenia gravis, myotonia congenita (including Thomsen's disease and Becker disease), nemaline myopathy, paramyotonia congenita, periodic paralysis (including hypokalemic and hyperkalemic), polymyositis, spinal and bulbar muscular atrophy, and spinal muscular atrophy.

In some embodiments the disease or disorder is suitable for treatment by an exon skipping therapeutic strategy. For example, in some embodiments, the subject to be treated has a disease or disorder selected from the group comprising Ataxia-telangiectasia, congenital disorder of glycosylation, fronto-temporal dementia and parkinsonism linked to chromosome 17, Niemann-Pick disease type C, neurofibromatosis type 1, neurofibromatosis type 2, megalencephalic leukoencephalopathy with subcortical cysts type 1, Pelizaeus-Merzbacher disease, familial dysautonomia, Marfan syndrome, and Loeys-Dietz syndrome.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

FIG. 1 is a schematic representation of the exonic structure of the human dystrophin gene. The dystrophin gene is the largest know protein coding human gene with 79 exons encoded by over 2 million base pairs.

FIGS. 2A-2D are schematic representations of frame shifting alterations in the human dystrophin gene and various genome editing strategies employed to correct them. FIG. 2A is a schematic representation of a frameshifting deletion of exon 50 within the dystrophin gene that results in a premature stop codon leading to a truncated and defective dystrophin protein. FIG. 2B depicts a single exon skipping strategy to correct the frameshifting alteration from FIG. 2A. Skipping of exon 51 by direct splicing of exon 49 to exon 52 prevents the frameshift and restores the reading frame of the dystrophin gene. FIG. 2C depicts a multiple exon skipping strategy to correct the frameshifting alteration from FIG. 2A. Skipping of exons 51-53 by direct splicing of exon 49 to exon 54 prevents the frameshift and restores the reading frame of the dystrophin gene. FIG. 2D depicts a repair strategy to correct the frameshifting alteration from FIG. 2A. A template with the correct wild type DNA sequence can be used to insert a correct copy of exon 50 through homology directed repair. This strategy will prevent the frameshift and lead to a functional, full length dystrophin protein product.

FIGS. 3A-3C are schematic representations of frame shifting alterations in the human dystrophin gene and various genome editing strategies employed to correct them. FIG. 3A is a schematic representation of a duplication of exon 2 of the dystrophin gene that results in a premature stop codon leading to a truncated and defective dystrophin protein. FIG. 3B shows an exon skipping strategy to correct the frameshifting alteration from FIG. 3A. The extra copy of exon 2 is skipped to prevent the frameshifting alteration. FIG. 3C shows an exon removal strategy in which the extra copy of exon 2 is deleted to prevent the frameshifting alteration from FIG. 3A.

FIG. 4 depicts a gene editing strategy to correct an R1667C mutation of the Ryr1 gene that occurs in central core disease. FIG. 4A is a genome browser view of a portion of the sequence of exon 34 of the human Ryr1 gene in which a C to T mutation effects a R1667C missense mutation. hs059742637 (5′-AGTGGAAGCGCTGCAGGTCCAGG-3′ (SEQ ID NO: 1) and hs059742644 (5′-CGCCCTGGGCAACAATCGCGTGG-3′ (SEQ ID NO: 2)) correspond to guide RNA sequences that direct the SpCas9 nickase to induce single strand breaks at DNA sites complementary to the guide RNA sequence. FIG. 4B depicts the overall strategy: a composition comprising an adeno-associated viral vector expressing the 2 guide RNAs and the SpCas9 nickase in combination with a second adeno-associated viral vector expressing a donor DNA template with the correct exon 34 sequence is used to mediate homology directed repair and correct the mutated Ryr1 gene.

FIG. 5 depicts a gene editing strategy to correct an T4709M mutation of the Ryr1 gene that occurs in central core disease. FIG. 5A is a genome browser view of a portion of the sequence of exon 96 of the human Ryr1 gene in which a C to T mutation effects a T4709M missense mutation. hs059750155 (5′-CTGGTGCTCAACACGCCGTAAGG-3′ (SEQ ID NO: 3)) and hs059750156 (5′-ACGGCGTGTTGAGCACCAGTCGG-3′ (SEQ ID NO: 4)) are exemplary guide RNA sequences that direct the SpCas9 nuclease to induce double strand breaks at DNA sites complementary to the guide RNA sequence. Either guide RNA can be used. FIG. 5B depicts the overall strategy: a composition comprising an adeno-associated viral vector expressing the guide RNA (e.g., hs059750155 or hs059750156) and the SpCas9 nuclease in combination with a second adeno-associated viral vector expressing a donor DNA template with the correct exon 96 sequence is used to mediate homology directed repair and correct the mutated Ryr1 gene.

FIG. 6A depicts a gene editing strategy to maintain the correct coding frame for nonsense mutations in exon 51 of DMD. The two targeting guide RNAs are indicated (hs132637319) and hs132637262), along with amplifying PCR primers. The entire amplicon is ˜1,450 bp, and the expected deletion from both guide RNAs is ˜630 bp. This deletion, resulting in exon-skipping is expected to maintain coding frame for non-sense mutations.

FIG. 6B depicts a PCR analysis following transfection indicates the deleted product at the expected size. FIG. 6C depicts a sequencing analysis which confirmed the deleted product following targeting.

FIG. 7A depicts an Interference of CRISPR Edits (ICE) analysis indicating cutting of the mouse DMD gene and mutations around the expected site (dotted line) following infection. AAV was used to deliver Cpf1 and a single guide RNA in mouse N2a cells. The WT sequence is indicated. FIG. 7B depicts an ICE analysis indicating cutting of the mouse DMD gene and mutations around the expected site (dotted line) following infection. AAV was used to deliver Cpf1 and a two targeting guide RNAs in mouse N2a cells: one that targets WT sequence (indicated), and one that targets the mdx mutation, a point mutation in the mouse DMD gene. The WT sequence is indicated. FIG. 7C depicts an ICE analysis indicating cutting of the mouse DMD gene and mutations around the expected site (dotted line) following transfection of MAD7 nuclease. The WT sequence is indicated.

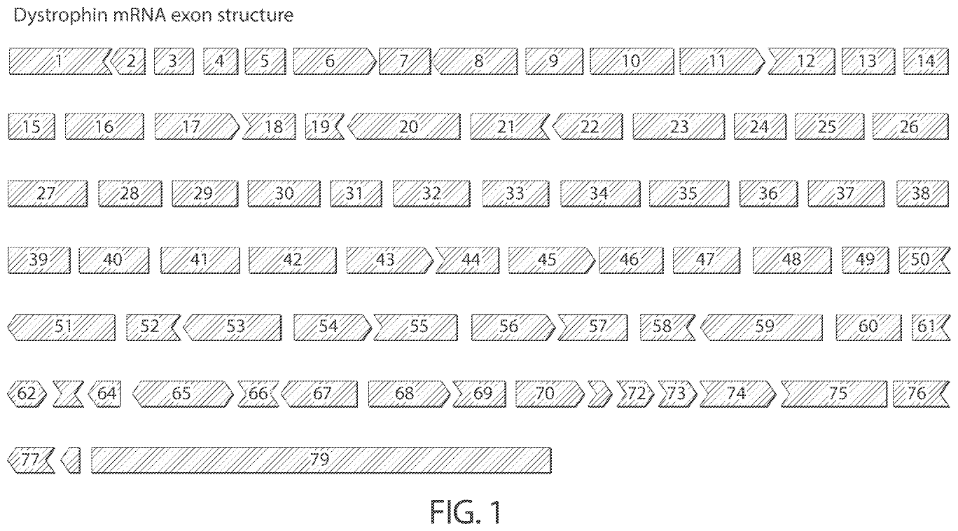

### DETAILED DESCRIPTION

The present disclosure is based in part, upon advancements in gene editing technology that have enhanced the ability to correct genetic defects in cells, thereby providing new avenues for treatment, as well as potential cures for genetic diseases or disorders. The present disclosure provides methods of treating subjects with genetic diseases or disorders through adeno-associated viral vector delivery of some CRIPSR systems (e.g., CRIPSR/Cas9 or CRIPSR/Cpf1) in order to edit the genomes of cells carrying defects in a mutated gene (e.g., dystrophin) to generate a precise corrective modification at the target locus, thereby restoring the activity of the mutated gene product. These and other aspects of the disclosure are set out in detail below.

### Definitions

The following definitions are included for the purpose of understanding the present subject matter and for constructing the appended patent claims.

Throughout the application, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

It should be understood that the expression “at least one of” includes individually each of the recited objects after the expression and the various combinations of two or more of the recited objects unless otherwise understood from the context and use.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

The use of the singular herein, for example, “a,” “an,” or “the,” includes the plural (and vice versa) unless specifically stated otherwise.

It should be understood that the order of steps or order for performing certain actions is immaterial so long as the present teachings remain operable. Moreover, two or more steps or actions can be conducted simultaneously.

At various places in the present specification, values are disclosed in groups or in ranges. It is specifically intended that the description include each and every individual subcombination of the members of such groups and ranges and any combination of the various endpoints of such groups or ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as,” “including,” or “for example,” is intended merely to better illustrate the present teachings and does not pose a limitation on the scope of the invention unless claimed. No language in the specification should be construed as indicating any non-claimed element as essential to the practice of the present teachings.

As used herein, the term “effective amount” or “amount sufficient” refers to the amount of a therapeutic composition or therapy that is sufficient to result in the prevention of the development, recurrence, or onset of a disease or disorder and one or more symptoms thereof, to reduce the severity or duration of a disease or disorder, to ameliorate one or more symptoms of a disease or disorder, to prevent the advancement of a disease or disorder, to cause regression of a disease or disorder, and/or to enhance or improve the therapeutic effect(s) of another therapy. In an embodiment, the “effective amount” or “therapeutically effective amount” refers to the amount of a composition that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. In a specific embodiment of the invention, a therapeutic composition in an “amount sufficient” refers to the amount of the composition needed to prevent, reduce, or alleviate at least one or more signs or symptoms of DMD, and relates to a sufficient amount of the composition to provide the desired effect, e.g., to treat a subject having DMD. The term “effective amount” therefore refers to an amount of a composition that is sufficient to promote a particular effect when administered to a typical subject, such as one who has or is at risk for DMD. An effective amount would also include an amount sufficient to delay the development of a symptom of the disease, alter the course of a symptom of the disease (for example but not limited to, slow the progression of a symptom of the disease), or reverse a symptom of the disease. It is understood that for any given case, an appropriate “effective amount” can be determined by one of ordinary skill in the art using routine experimentation.

The terms “subject” and “patient” are used interchangeably herein. The subject treated by the presently disclosed methods in their many embodiments is desirably a human subject, although it is to be understood that the methods described herein are effective with respect to all vertebrate species which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, humans, monkeys, apes, and the like; bovines, e.g., cattle, oxen, and the like; ovines, sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease.

As used herein, the terms “treat,” “treatment,” and “treating” in the context of the administration of a therapeutic composition to a subject refer to the reversing, reduction or inhibition of the progression and/or duration of the disease, preventing or reducing the likelihood of the disease, reduction or amelioration of the severity, and/or the amelioration of one or more symptoms of the disease, disorder, or condition to which such term applies resulting from the administration of one or more therapies. In some embodiments, the treatment reduces the amount of defective dystrophin protein in cells (e.g., muscle cells) of the subject. For example, the treatment can reduce the amount of defective dystrophin protein in cells (e.g., muscle cells) of the subject by at least 5%, 10%, 15%, 20%, 25%, 30%, 33%, 35%, 40%, 45%, 50%, 55%, 60%, 70%, 75%, 80%, 85%, 90%, 91%, 92%, 93%, 94%, 95%, 96%, 97%, 98%, 99% or more as compared to the amount of defective dystrophin in cells in a subject before undergoing treatment or in a subject who does not undergo treatment. In some embodiments, the treatment increases the amount of dystrophin protein activity in cells (e.g., muscle cells) of the subject. For example, the treatment can increase the amount of dystrophin protein activity in by at least 5%, 10%, 15%, 20%, 25%, 30%, 33%, 35%, 40%, 45%, 50%, 55%, 60%, 70%, 75%, 80%, 85%, 90%, 91%, 92%, 93%, 94%, 95%, 96%, 97%, 98%, 99% or more as compared to the amount of dystrophin protein activity in cells in a subject before undergoing treatment or in a subject who does not undergo treatment.

As used herein, the terms “manage,” “managing,” and “management” in the context of the administration of a therapy to a subject refer to the beneficial effects that a subject derives from a therapy (e.g., a prophylactic or therapeutic composition) or a combination of therapies, while not resulting in a cure of the disease or disorder. In certain embodiments, a subject is administered one or more therapies (e.g., one or more prophylactic or therapeutic compositions) to “manage” DMD so as to prevent the progression or worsening of the condition.

As used herein, the terms “prevent,” “preventing” and “prevention” in the context of the administration of a therapy to a subject refer to the prevention or inhibition of the recurrence, onset, and/or development of a disease or disorder, or a symptom thereof in a subject resulting from the administration of a therapy (e.g., a prophylactic or therapeutic composition), or a combination of therapies (e.g., a combination of prophylactic or therapeutic compositions). In some embodiments, such terms refer to one, two, three, or more results following the administration of one or more therapies: (1) a delay in the development of a symptom of the disease, (2) an alteration of the course of a symptom of the disease (for example but not limited to, slowing the progression of a symptom of the disease), and (3) a reverse of a symptom of the disease. In some embodiments, such terms refer to a reduction in mortality and/or an increase in survival rate of a patient population. In further embodiments, such terms refer to an increase or enhancement in the quality of life of a patient population. In some embodiments, such terms refer to a decrease in hospitalization rate of a patient population and/or a decrease in hospitalization length for a patient population.

As used herein, the terms “therapies” and “therapy” can refer to any method(s), composition(s), and/or agent(s) that can be used in the prevention, treatment and/or management of a disease, disorder or condition, or one or more symptoms thereof. In certain embodiments, the terms “therapy” and “therapies” refer to steroid therapy, physical therapy, gene therapy, chemotherapy, small molecule therapy, radioimmunotherapy, toxin therapy, prodrug-activating enzyme therapy, biologic therapy, antibody therapy, surgical therapy, hormone therapy, immunotherapy, anti-angiogenic therapy, targeted therapy, epigenetic therapy, demethylation therapy, histone deacetylase inhibitor therapy, differentiation therapy, radiation therapy, or a combination of the foregoing and/or other therapies useful in the prevention, management and/or treatment of a disease, disorder or condition, or one or more symptoms thereof.

As used herein, the terms “administering” and “administered” refer to the delivery of a composition into a subject by a method or route that results in at least partial localization of the composition at a desired site. A composition can be administered by any appropriate route that results in effective treatment in the subject, i.e. administration results in delivery to a desired location in the subject where at least a portion of the composition is delivered to the desired site for a period of time. Modes of administration include injection, infusion, instillation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some examples, the route is intravenous.

### Methods of Treatment

In one aspect, the present disclosure provides a method for treating a subject (e.g., a human) with Duchenne muscular dystrophy (DMD) By using gene editing tools to create permanent changes to the genome that can restore the dystrophin reading frame and restore the dystrophin protein activity, the resulting therapy can correct the underlying genetic defect causing the disease. In some embodiments, the present disclosure provides a method of treating a subject with Duchenne muscular dystrophy (DMD), comprising administering to the subject a composition comprising an adeno-associated viral vector, an RNA-guided nuclease or nickase or a DNA endonuclease, and at least one dystrophin-targeted guide RNA in amount sufficient to delete a skippable exon encoding the dystrophin gene, wherein the subject with DMD has a mutation causing a frameshift in the dystrophin gene and wherein the deletion of the skippable exon prevents the frameshift.

In some embodiments, preventing the frameshift in the dystrophin gene results in partial or complete restoration of dystrophin protein activity. In some embodiments, preventing the frameshift in the dystrophin gene results in partial restoration of dystrophin protein activity. For example, in some embodiments, preventing the frameshift in the dystrophin gene increases the amount of dystrophin protein activity in the subject's cells (e.g., muscle cells). For example, the amount of dystrophin protein activity in the subject's cells (e.g., muscle cells) increases by at least 5%, 10%, 15%, 20%, 25%, 30%, 33%, 35%, 40%, 45%, 50%, 55%, 60%, 70%, 75%, 80%, 85%, 90%, 91%, 92%, 93%, 94%, 95%, 96%, 97%, 98%, 99% or more as compared to the amount of dystrophin protein activity in such cells in a subject before undergoing treatment or in a subject who does not undergo treatment. In some embodiments, preventing the frameshift in the dystrophin gene results in complete restoration of dystrophin protein activity.

In some embodiments, the DNA endonuclease is a Cas9 endonuclease or a Cpf1 endonuclease. For example, in some embodiments, the DNA endonuclease is a Cas9 endonuclease. In another embodiment, the DNA endonuclease is a Cpf1 endonuclease.

In some embodiments, the composition comprises at least one (e.g., 1, 2, 3, 4, 5 or more) dystrophin-targeted guide RNA. In some embodiments, the composition comprises one dystrophin-targeted guide RNA. In some embodiments, the composition comprises two dystrophin-targeted guide RNAs. In some embodiments, the composition comprises more than two dystrophin-targeted guide RNAs. In some embodiments, the dystrophin-targeted guide RNAs recognize different DNA sequences (also referred to as target sites). For example, in some embodiments, the composition comprises two dystrophin-targeted guide RNAs that recognize different target sites. The target site(s) recognized by the at least one dystrophin targeted guide RNA may occur in an intron and/or exon of the dystrophin gene. In some embodiments for example, the target site(s) recognized by the at least one dystrophin targeted guide RNA may occur in an intron of the dystrophin gene. In some embodiments, the composition comprises two dystrophin-targeted guide RNAs that recognize different intronic target sites. In some embodiments, the composition comprises two dystrophin-targeted guide RNAs wherein at least one guide RNA recognizes an intron target site.

Exon skipping is a strategy in which sections of genes are “skipped” during pre-mRNA splicing, to enlarge a deletion so that it becomes its nearest in-frame counterpart. In the case of DMD, this allows for the creation of a partially deleted but functional dystrophin protein (van Deutekom J, 2003). The exon skipping approach has been employed through the use of antisense oligonucleotide (AON) to modulate splicing by hiding specific sites essential for exon inclusion from the splicing machinery. However AON-mediated exon skipping has met limited success because it suffers from inefficiency of oligonucleotide tissue uptake, requirement for lifelong delivery of oligonucleotides and incomplete exon skipping. The present disclosure circumvents this challenge by providing methods that use the CRISPR systems (e.g., CRISPR/Cas9 or CRISPR/Cpf1) to destroy exon splice sites preceding DMD mutations or to delete mutant or out-of-frame exons, thereby allowing splicing between surrounding exons to recreate an in-frame dystrophin protein that lacks the mutations.

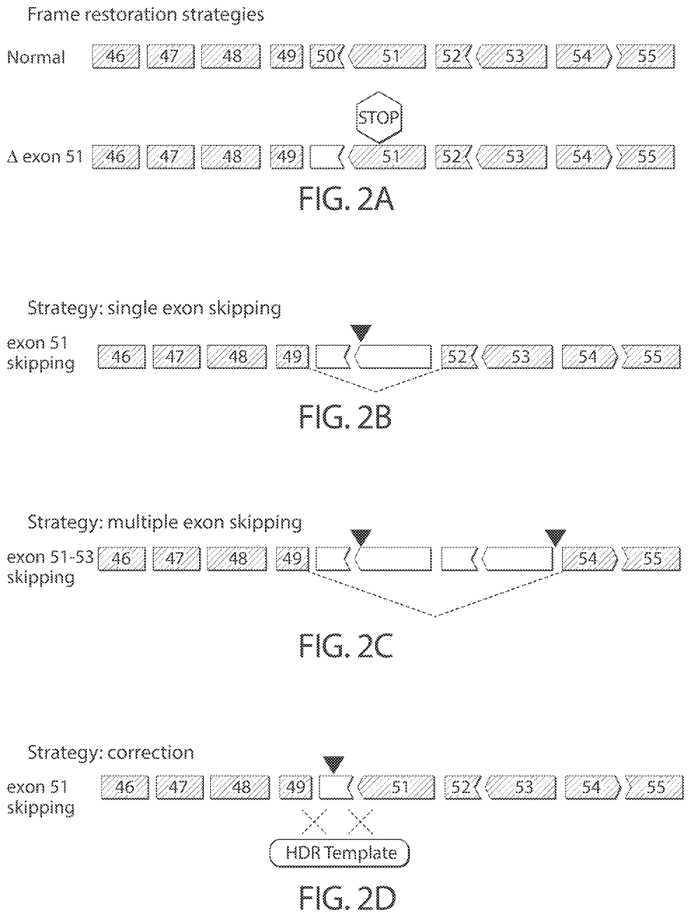

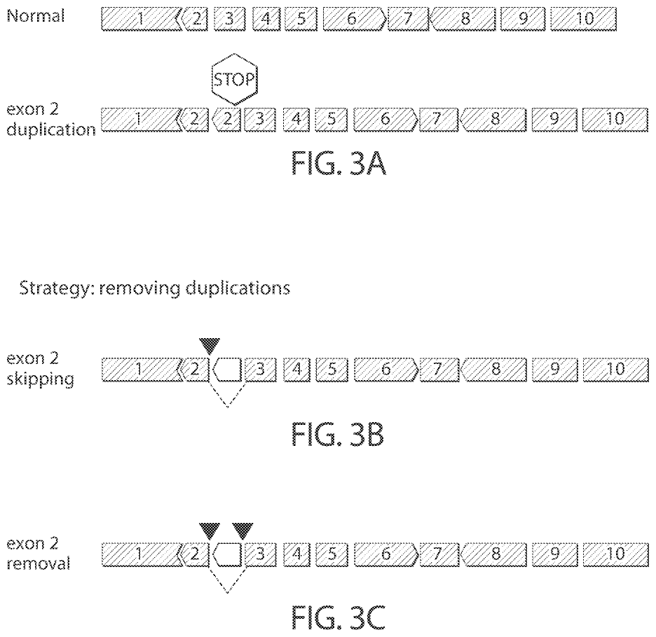

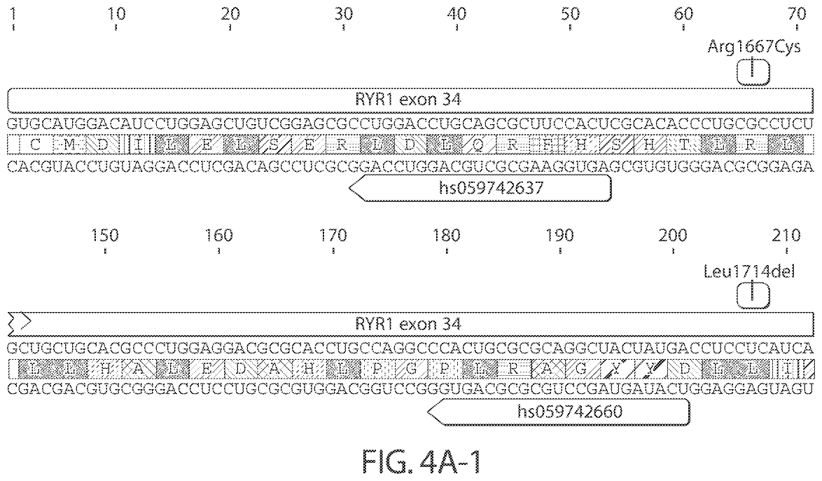

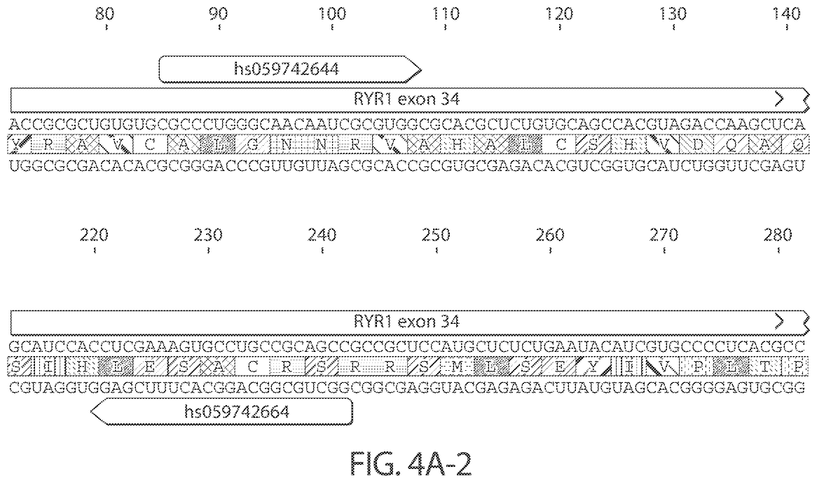

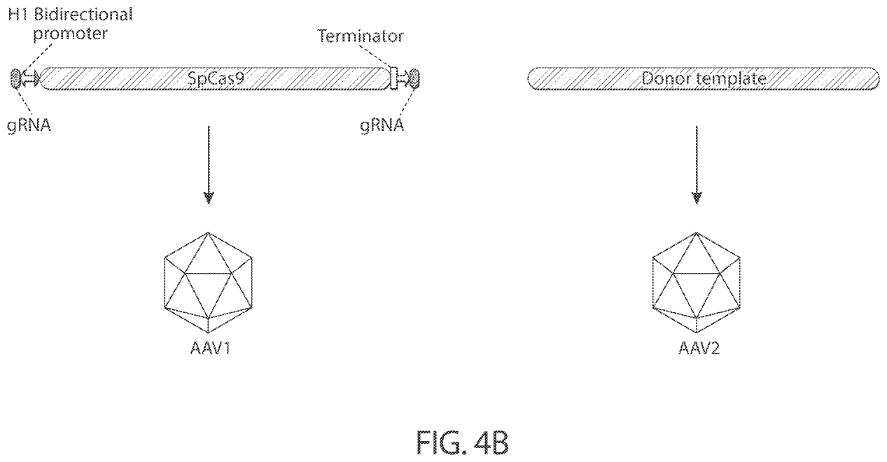

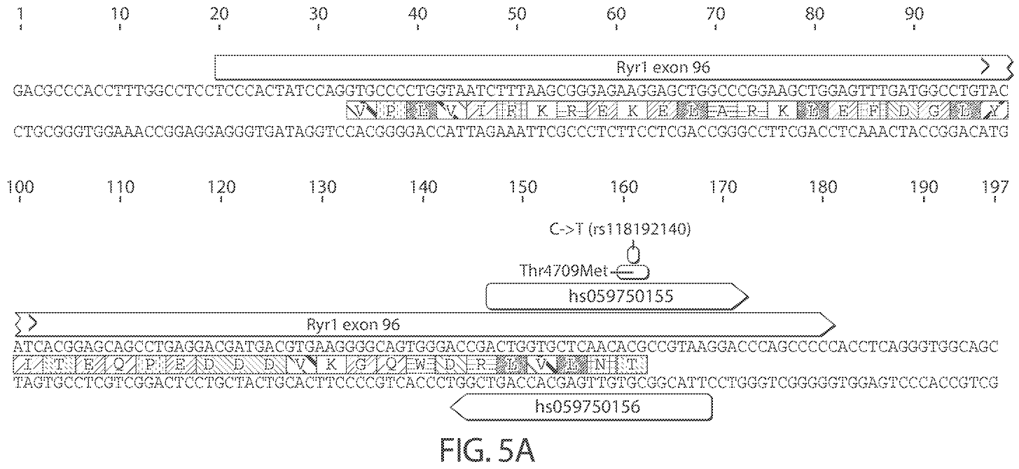

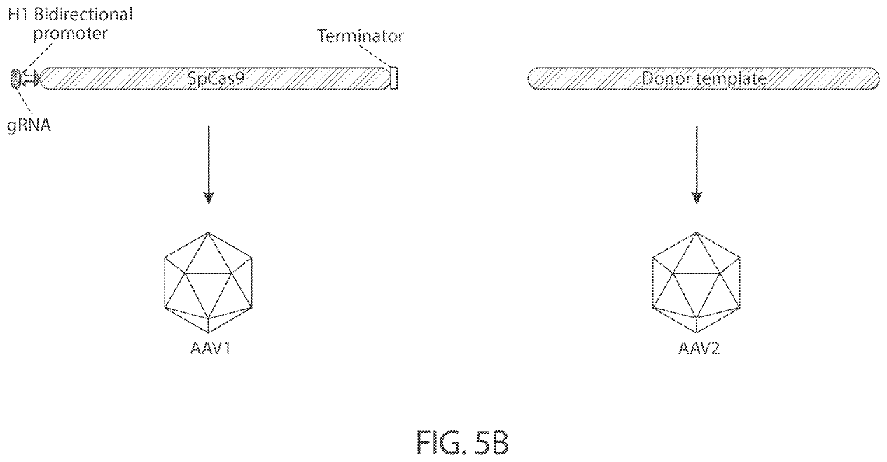

In some embodiments, the disclosed method comprises deletion of one or more (e.g., 1, 2, 3, 4, 5, 6, 7, 8, 9, 10 or more) of any of the 79 exons in the dystrophin gene. For example, in some embodiments, the disclosed method comprises deletion of one exon (for example, refer to FIG. 2B) in the dystrophin gene. In some embodiments, for example, the disclosed method comprises deletion of two exons in the dystrophin gene. In some embodiments, for example, the disclosed method comprises deletion of three exons in the dystrophin gene (for example, refer to FIG. 2C). In some embodiments, for example, the disclosed method comprises deletion of more than three of any of the 79 exons in the dystrophin gene.

Assessment of the range of mutations that cause DMD shows that the skipping of any exon could be an effective therapy for many different DMD causing mutations in the dystrophin gene. It is estimated that the skipping of only 12 different exons could theoretically correct approximately 75% of DMD causing mutations (van Deutekom J, 2003). Accordingly, in some embodiments, the disclosed method comprises deletion of one or more (e.g., 1, 2, 3, 4, 5, 6, 7, 8, 9, 10, 11 or 12) skippable dystrophin exons selected from the group consisting of exon 2, exon 8, exon 17, exon 43, exon 44, exon 45, exon 46, exon 50, exon 51, exon 52, exon 53, and exon 55. For example, in some embodiments, the disclosed method comprises deletion of one skippable dystrophin exon selected from the group consisting of exon 2, exon 8, exon 17, exon 43, exon 44, exon 45, exon 46, exon 50, exon 51, exon 52, exon 53, and exon 55. In some embodiments, the disclosed method comprises deletion of two skippable dystrophin exons selected from the group consisting of exon 2, exon 8, exon 17, exon 43, exon 44, exon 45, exon 46, exon 50, exon 51, exon 52, exon 53, and exon 55. In some embodiments, the disclosed method comprises deletion of three skippable dystrophin exons selected from the group consisting of exon 2, exon 8, exon 17, exon 43, exon 44, exon 45, exon 46, exon 50, exon 51, exon 52, exon 53, and exon 55. In some embodiments, the disclosed method comprises deletion of more than three skippable dystrophin exons selected from the group consisting of exon 2, exon 8, exon 17, exon 43, exon 44, exon 45, exon 46, exon 50, exon 51, exon 52, exon 53, and exon 55.

In some embodiments, the skippable exon in the dystrophin gene is selected from the list in Table 1, based on the deleted exon or deleted exons that specifically occur in the dystrophin gene of the subject.

TABLE 1
Therapeutic exon skipping strategies for DMD deletions

| Skippable | Dystrophin |
|---|---|
| Exon | exon deletions |
| exon 2 | 3-7, 3-19, 3-21 |
| exon 8 | 3-7, 4-7, 5-7, 6-7 |
| exon 17 | 12-16, 18-33, 18-41, 18-44 |
| exon 43 | 44, 44-47, 44-49, 44-51 |
| exon 44 | 14-43, 19-43, 30-43, 35-43, 36-43, 40-43, 42-43, 45, 45-54 |
| exon 45 | 12-44, 18-44, 44, 46-47, 46-48, 46-49, 46-51, 46-53, 46-55 |
| exon 46 | 21-45, 45, 47-54, 47-56 |
| exon 50 | 51, 51-53, 51-55 |
| exon 51 | 45-50, 47-50, 48-50, 49-50, 50, 52, 52-63 |
| exon 52 | 51, 53, 53-55 |
| exon 53 | 10-52, 45-52, 46-52, 47-52, 48-52, 49-52, 50-52, 52 |
| exon 55 | 45-54, 48-54 |

In some embodiments, the present disclosure provides a method of treating a subject (e.g., a human) with Duchenne muscular dystrophy (DMD), wherein the subject has a mutation that causes a frameshift in the dystrophin gene. There are a variety of frameshifting mutations that occur in dystrophin gene which are known to cause DMD. Non-limiting examples of such mutations in the dystrophin gene include a deletion, an insertion, a duplication, or a translocation. The most prevalent cause of DMD is deletions of one or more exons; approximately 60-70% of DMD cases are caused by large deletions (van Deutekom J, 2003). In some embodiments, the subject with DMD (e.g., a human) has one or more of a deletion, an insertion, a duplication, or a translocation in their dystrophin gene. For example, in some embodiments, the subject with DMD (e.g., a human) has a deletion and/or a duplication in the dystrophin gene. In a specific embodiment, the subject with DMD (e.g., a human) has a deletion in their dystrophin gene.

In some embodiments, the subject with DMD (e.g., a human) has a mutation (e.g., a deletion, an insertion, a duplication, or a translocation) in their dystrophin gene, wherein the mutation is in exons 3-7, 3-19, 3-21, 4-7, 5-7, 6-7, 12-16, 18-33, 18-41, 18-44, 44, 44-47, 44-49, 44-51, 14-43, 19-43, 30-43, 35-43, 36-43, 40-43, 42-43, 45, 45-54, 12-44, 18-44, 46-47, 46-48, 46-49, 46-51, 46-53, 46-55, 21-45, 47-54, 47-56, 51, 51-53, 51-55, 45-50, 47-50, 48-50, 49-50, 50, 52, 52-63, 53, 53-55, 10-52, 45-52, 46-52, 47-52, 48-52, 49-52, 50-52, 45-54, or 48-54. In some embodiments, the subject with DMD (e.g., a human) has a mutation in their dystrophin gene, wherein the mutation is a deletion of exons 3-7, 3-19, 3-21, 4-7, 5-7, 6-7, 12-16, 18-33, 18-41, 18-44, 44, 44-47, 44-49, 44-51, 14-43, 19-43, 30-43, 35-43, 36-43, 40-43, 42-43, 45, 45-54, 12-44, 18-44, 46-47, 46-48, 46-49, 46-51, 46-53, 46-55, 21-45, 47-54, 47-56, 51, 51-53, 51-55, 45-50, 47-50, 48-50, 49-50, 50, 52, 52-63, 53, 53-55, 10-52, 45-52, 46-52, 47-52, 48-52, 49-52, 50-52, 45-54, or 48-54.

In another aspect, the present disclosure provides a method of treating a subject (e.g., a human) with Duchenne muscular dystrophy (DMD), comprising administering to the subject a composition comprising an adeno-associated viral vector, an RNA-guided nuclease or nickase or a DNA endonuclease, and at least one dystrophin targeted guide RNA in amount sufficient to restore the reading frame of the dystrophin gene in the subject; wherein the dystrophin targeted guide RNA recognizes a target site in the dystrophin gene selected from the sequences identified in Table 2.

In some embodiments, restoration of the reading frame of the dystrophin gene results in partial or complete restoration of dystrophin protein activity. In some embodiments, restoration of the reading frame of the dystrophin gene results in partial restoration of dystrophin protein activity. For example, in some embodiments, restoration of the reading frame of the dystrophin gene increases the amount of dystrophin protein activity in the subject's cells (e.g., muscle cells). For example, the amount of dystrophin protein activity in the subject's cells (e.g., muscle cells) increases by at least 5%, 10%, 15%, 20%, 25%, 30%, 33%, 35%, 40%, 45%, 50%, 55%, 60%, 70%, 75%, 80%, 85%, 90%, 91%, 92%, 93%, 94%, 95%, 96%, 97%, 98%, 99% or more as compared to the amount of dystrophin protein activity in such cells in a subject before undergoing treatment or in a subject who does not undergo treatment. In some embodiments, restoration of the reading frame of the dystrophin gene results in complete restoration of dystrophin protein activity.

In some embodiments, the DNA endonuclease is a Cas9 endonuclease, a Cpf1 endonuclease, or a MAD7 endonuclease. For example, in some embodiments, the DNA endonuclease is a Cas9 endonuclease. In another embodiment, the DNA endonuclease is a Cpf1 endonuclease. In another embodiment, the DNA endonuclease is a MAD7 endonuclease.

In some embodiments, the composition comprises at least one (e.g., 1, 2, 3, 4, 5 or more) dystrophin-targeted guide RNA. In some embodiments, the composition comprises one dystrophin-targeted guide RNA. In some embodiments, the composition comprises two dystrophin-targeted guide RNAs. In some embodiments, the composition comprises more than two dystrophin-targeted guide RNAs. In some embodiments, the dystrophin-targeted guide RNAs recognize different DNA sequences (also referred to as target sites). For example, in some embodiments, the composition comprises two dystrophin-targeted guide RNAs that recognize different target sites. The target site(s) recognized by the at least one dystrophin targeted guide RNA may occur in an intron and/or exon of the dystrophin gene. In some embodiments for example, the target site(s) recognized by the at least one dystrophin targeted guide RNA may occur in an intron of the dystrophin gene. In some embodiments, the composition comprises two dystrophin-targeted guide RNAs that recognize different intronic target sites. In some embodiments, the composition comprises two dystrophin-targeted guide RNAs wherein at least one guide RNA recognizes an intron target site. Non-limiting examples of intronic targets sites within the dystrophin gene are identified in Table 2.

In yet another aspect, the present disclosure provides a method of treating a subject (e.g., a human) with a genetic disease or disorder, comprising a gene editing strategy comprising administering to the subject a composition comprising an adeno-associated viral vector, an RNA-guided nuclease or nickase or a DNA endonuclease, at least one guide RNA, and optionally a donor DNA template in an amount sufficient to (i) delete a skippable exon encoding a mutated gene, wherein such deletion prevents a frameshift of the mutated gene; and/or (ii) correct the DNA sequence of the mutated gene via homology directed repair (HDR) wherein the mutated gene is associated with the cause of the disease or disorder; and wherein said method of treatment reduces at least one symptom associated with the disease or disorder.

In some embodiments, the present disclosure provides a method of treating a subject (e.g., a human) with a genetic disease or disorder, comprising a gene editing strategy comprising administering to the subject a composition comprising an adeno-associated viral vector, a DNA endonuclease, at least one guide RNA, and a donor DNA template in an amount sufficient to (i) delete a skippable exon encoding a mutated gene, wherein such deletion prevents a frameshift of the mutated gene; and/or (ii) correct the DNA sequence of the mutated gene via homology directed repair (HDR) wherein the mutated gene is associated with the cause of the disease or disorder; and wherein said method of treatment reduces at least one symptom associated with the disease or disorder. In some embodiments, the present disclosure provides a method of treating a subject (e.g., a human) with a genetic disease or disorder, comprising a gene editing strategy comprising administering to the subject a composition comprising an adeno-associated viral vector, a DNA endonuclease, and at least one guide RNA in an amount sufficient to (i) delete a skippable exon encoding a mutated gene, wherein such deletion prevents a frameshift of the mutated gene; wherein the mutated gene is associated with the cause of the disease or disorder; and wherein said method of treatment reduces at least one symptom associated with the disease or disorder.

In some embodiments of the foregoing methods of treatment, the DNA endonuclease is a Cas9 endonuclease, a Cpf1 endonuclease, or a MAD7 endonuclease. For example, in some embodiments, the DNA endonuclease is a Cas9 endonuclease. In other embodiments, the DNA endonuclease is a Cpf1 endonuclease. In other embodiments, the DNA endonuclease is a MAD7 endonuclease.

In some embodiments of the foregoing methods of treatment, the composition comprises at least one (e.g., 1, 2, 3, 4, 5 or more) guide RNA. In some embodiments, the composition comprises one guide RNA. In some embodiments, the composition comprises two guide RNAs. In some embodiments, the composition comprises more than two guide RNAs. In some embodiments, the guide RNAs recognize different DNA sequences (also referred to as target sites). For example, in some embodiments, the composition comprises two guide RNAs that recognize different target sites. The target site(s) recognized by the at least one guide RNA may occur in an intron and/or exon of the mutated gene associated with the cause of the disease or disorder. In some embodiments for example, the target site(s) recognized by the at least one guide RNA may occur in an intron of the mutated gene. In some embodiments, the composition comprises two guide RNAs that recognize different intronic target sites. In some embodiments, the composition comprises two guide RNAs wherein at least one guide RNA recognizes an intron target site. Non-limiting examples of intronic targets sites within the dystrophin gene, which is mutated in DMD for example, are identified in Table 2.

In some embodiments of the foregoing method of treatment, the composition comprises at least one (e.g., 1, 2, 3, 4, 5 or more) guide RNA that recognizes a target site in a gene selected from the group consisting of MYH7, TNNT2, TPM1, MYBPC3, PRKAG2, TNNI3, MYL3, TNN, MYL2, ACTC1, CSRP3, TNNC1, MYH6, VCL, MYOZ2, JPH2, PLN, CALR3, NEXN, MYPN, ACTN2, LDB3, TCAP, FLNC, SOD1, C9ORF72, and RYR. In some embodiments, the composition comprises one guide RNA that recognizes a target site in a gene selected from the group consisting of MYH7, TNNT2, TPM1, MYBPC3, PRKAG2, TNNI3, MYL3, TNN, MYL2, ACTC1, CSRP3, TNNC1, MYH6, VCL, MYOZ2, JPH2, PLN, CALR3, NEXN, MYPN, ACTN2, LDB3, TCAP, FLNC, SOD1, C9ORF72, and RYR. In some embodiments, the composition comprises two guide RNAs that recognize a target site in a gene selected from the group consisting of MYH7, TNNT2, TPM1, MYBPC3, PRKAG2, TNNI3, MYL3, TNN, MYL2, ACTC1, CSRP3, TNNC1, MYH6, VCL, MYOZ2, JPH2, PLN, CALR3, NEXN, MYPN, ACTN2, LDB3, TCAP, FLNC, SOD1, C9ORF72, and RYR. In some embodiments, the composition comprises more than two guide RNAs that recognize a target site in a gene selected from the group consisting of MYH7, TNNT2, TPM1, MYBPC3, PRKAG2, TNNI3, MYL3, TNN, MYL2, ACTC1, CSRP3, TNNC1, MYH6, VCL, MYOZ2, JPH2, PLN, CALR3, NEXN, MYPN, ACTN2, LDB3, TCAP, FLNC, SOD1, C9ORF72, and RYR.

In some embodiments of the foregoing method of treatment, the composition optionally comprises a donor DNA template to correct the DNA sequence of the mutated gene via a homology directed repair mechanism. When an endonuclease cleaves DNA to effect a double-strand break, such breaks can be repaired by natural, endogenous cellular processes such as homology-directed repair (HDR) and non-homologous end-joining (NHEJ). NHEJ directly joins the DNA ends resulting from a double-strand break without the need for a homologous template. NHEJ is an error-prone repair mechanism that can sometimes result in the loss or addition of nucleotide sequence (small deletions or insertions known as indels), which can disrupt or otherwise alter gene expression. HDR, on the other hand, utilizes a homologous sequence, or donor sequence, as a template for inserting a defined DNA sequence at the break point.

The donor sequence can be in the endogenous genome, such as a sister chromatid. Alternatively, the donor can be an exogenous nucleic acid, such as a plasmid, a single-strand oligonucleotide, a double-strand oligonucleotide, a duplex oligonucleotide or a virus, that has regions of high homology with the endonuclease-cleaved locus, but which can also contain additional sequence or sequence changes including deletions that can be incorporated into the cleaved target locus. For the purposes of gene editing as described herein, the repair template can be supplied as an exogenous nucleic acid, such as a plasmid, duplex oligonucleotide, single-strand oligonucleotide or viral nucleic acid.

The donor template may comprise at least a portion of the wild-type sequence of the mutated gene. In this way, a corrected version of the mutated gene can be introduced between the flanking regions of homology so that the corrected nucleic acid sequence becomes incorporated into the target locus. Hence, using the exogenous donor template, HDR results in permanent insertion or correction of a mutated gene, thereby leading to rescued expression and activity of the causative gene, and subsequently, reduction of at least one symptom associated with the disease or disorder. One skilled in the art would appreciate that with other exogenous donor templates, a non-wild-type nucleic acid sequence (such as a transgene) or modification (such as a single or multiple base change or a deletion) can be introduced between the flanking regions of homology so that the additional or altered nucleic acid sequence also becomes incorporated into the target locus. The present disclosure also contemplates such non-wild-type corrections or modifications.

In some embodiments, the composition optionally comprises a donor DNA template to correct the DNA sequence of the mutated gene via a homology directed repair mechanism. In some embodiments, the donor DNA template is homologous to a gene selected from the group consisting of DMD, MYH7, TNNT2, TPM1, MYBPC3, PRKAG2, TNNI3, MYL3, TNN, MYL2, ACTC1, CSRP3, TNNC1, MYH6, VCL, MYOZ2, JPH2, PLN, CALR3, NEXN, MYPN, ACTN2, LDB3, TCAP, FLNC, SOD1, C9ORF72, and RYR. For example, in some embodiments, the donor DNA template is homologous to RYR. In some embodiments, the donor DNA template is homologous to the dystrophin gene (DMD). In some embodiments, the donor DNA template is homologous to DMD and provides a portion of the wild-type DMD sequence. In some embodiments, the donor DNA template comprises at least a part of exon 1, exon 2, exon 3, exon 4, exon 5, exon 6, exon 7, exon 8, exon 9, exon 10, exon 1 1, exon 12, exon 13, exon 14, exon 15, exon 16, exon 17, exon 18, exon 19, exon 20, exon 21 exon 22, exon 23, exon 24, exon 25, exon 26, exon 27, exon 28, exon 29, exon 30, exon 31 exon 32, exon 33, exon 34, exon 35, exon 36, exon 37, exon 38, exon 39, exon 40, exon 41 exon 42, exon 43, exon 44, exon 45, exon 46, exon 47, exon 48, exon 49, exon 50, exon 51 exon 52, exon 53, exon 54, exon 55, exon 56, exon 57, exon 58, exon 59, exon 60, exon 61 exon 62, exon 63, exon 64, exon 65, exon 66, exon 67, exon 68, exon 69, exon 70, exon 71 exon 72, exon 73, exon 74, exon 75, exon 76, exon 77, exon 78, exon 79, intronic regions, synthetic intronic regions, fragments, combinations thereof, or the entire dystrophin gene.

In certain embodiments, the present disclosure provides a method of treating a subject (e.g., a human) with a genetic disease or disorder, such as, for example Duchenne muscular dystrophy. In some embodiments, the genetic disease or disorder is a muscle-related disease or disorder selected from the group consisting of Hypertrophic cardiomyopathy (HCM or CMH), amyotrophic lateral sclerosis, Becker's muscular dystrophy, central core disease, centronuclear myopathy (including myotubular myopathy), Charcot-Marie-Tooth disease, congenital muscular dystrophy, congenital myasthenic syndrome, Dejerine-Sottas disease, dermatomyositis, Duchenne muscular dystrophy, Emery-Dreifuss muscular dystrophy, facioscapulohumeral muscular dystrophy, Friedreich's ataxia, hyperthyroid myopathy, hypothyroid myopathy, inclusion body myositis, Lambert-Eaton myasthenic syndrome, Limb-girdle muscular dystrophy, mitochondrial myopathy, myasthenia gravis, myotonia congenita (including Thomsen's disease and Becker disease), nemaline myopathy, paramyotonia congenita, periodic paralysis (including hypokalemic and hyperkalemic), polymyositis, spinal and bulbar muscular atrophy, and spinal muscular atrophy.

In certain embodiments, the subject to be treated (e.g., a human) has a disease or disorder that is suitable for treatment by an exon skipping therapeutic strategy. As discussed earlier, exon skipping is a strategy in which sections of genes are “skipped” during pre-mRNA splicing, to enlarge a deletion so that it becomes its nearest in-frame counterpart. In the case of DMD, this allows for the creation of a partially deleted but functional dystrophin protein (van Deutekom J, 2003). In certain embodiments, exon skipping is effected by deletion of at least a portion of the targeted exon. For example, in certain embodiments, in DMD exon skipping is effected by deletion of at least a portion of one or more (e.g., 1, 2, 3, 4, 5, 6, 7, 8, 9, 10, 11 or 12) dystrophin exons selected from the group consisting of exon 2, exon 8, exon 17, exon 43, exon 44, exon 45, exon 46, exon 50, exon 51, exon 52, exon 53, and exon 55. In any disease or disorder suitable for treatment by an exon skipping therapeutic strategy, exon skipping would be directed to restoring the reading frame of a mutated gene associated with the cause of the disease or disorder.

Accordingly, in certain embodiments, the subject to be treated (e.g., a human) has a disease or disorder selected from the group comprising Ataxia-telangiectasia, congenital disorder of glycosylation, fronto-temporal dementia and parkinsonism linked to chromosome 17, Niemann-Pick disease type C, neurofibromatosis type 1, neurofibromatosis type 2, megalencephalic leukoencephalopathy with subcortical cysts type 1, Pelizaeus-Merzbacher disease, familial dysautonomia, Marfan syndrome, and Loeys-Dietz syndrome.

### Endonucleases and RNA-Guided Nucleases

In some embodiments, the composition administered in a disclosed method of treating subject (e.g., a human) comprises a DNA endonuclease. The DNA endonuclease can be any endonuclease that is capable of cleaving DNA to effect a single or double strand break at the intended locus. For example, the DNA endonuclease can be a MAD1, MAD2, MAD3, MAD4, MAD5, MAD6, MAD7, MAD5, MAD5 MAD10, MAD11, or MAD11 endonuclease (see, e.g., U.S. Pat. No. 9,982,279). The DNA endonuclease can be a Cas1, Cas1 B, Cas2, Cas3, Cas4, Cas5, Cas6, Cas7, Cas8, Cas9 (also known as Csn1 and Csx12), Cas1 OO, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease; a homolog thereof, a recombinant of the naturally occurring molecule thereof, a codon-optimized version thereof, a modified version thereof (e.g., a mutated variant such as a nickase), and combinations of any of the foregoing. For example, in some embodiments, the DNA endonuclease is a Cas9 or Cpf1 endonuclease that effects a single-strand break (SSB) or double-strand break (DSB) at a locus within or near the mutated gene (e.g., the dystrophin gene).

In some embodiments, the DNA endonuclease is a Cas9 endonuclease (e.g., a recombinant Cas9, a codon-optimized Cas9, a modified or mutated Cas9). The Cas9 endonuclease can be derived from a variety of bacterial species. For example, in certain embodiments, the Cas9 endonuclease is derived from Streptococcus thermophiles, Streptococcus pyogenes, Neisseria meningitides, Staphylococcus aureus, or Treponema denticola. In a specific embodiment, the Cas9 endonuclease is derived from Staphylococcus aureus (SaCas9). In another specific embodiment, the Cas9 endonuclease is derived from Streptococcus pyogenes (SpCas9). Wild type Cas9 has two active sites (RuvC and HNH nuclease domains) for cleaving DNA, one for each strand of the double helix. However, nickase variants of Cas9 are readily available (e.g., Addgene, plasmid #: 48873) that are only capable of cleaving one strand of the DNA due to catalytic inactivation of the RuvC or HNH nuclease domains. Accordingly, in a specific embodiment, the Cas9 endonuclease is a mutated SpCas9 endonuclease (e.g., a nickase) and/or a codon-optimized version thereof.

In other embodiments, the DNA endonuclease is a Cpf1 endonuclease (e.g., a recombinant Cpf1, a codon-optimized Cpf1, a modified or mutated Cpf1). The Cpf1 endonuclease can be derived from a variety of bacterial species. For example, in certain embodiments, the Cpf1 endonuclease is derived from Acidaminococcus bacteria or Lachnospiraceae bacteria. In a specific embodiment, the Cpf1 endonuclease is a Lachnospiraceae bacterium ND2006 Cpf1.

In other embodiments, the DNA endonuclease is a MAD7 endonuclease (e.g., a recombinant MAD7, a codon-optimized MAD7, a modified or mutated MAD7). MAD7 is a codon optimized endonuclease can be derived from Eubacterium rectale (Inscripta, Boulder, Colo.) MAD7 is described in U.S. Pat. No. 9,982,279.

In other embodiments, an RNA-guided nuclease is used. Exemplary RNA-guided nucleases include Cas13a, Cas 13b and Cas13d.

### Guide RNAs

In addition to an endonuclease, methods of treatment presently disclosed also comprise a guide RNA to recruit and direct the endonuclease activity to the locus of a mutated gene, such as a gene associated with the cause of the disease or disorder (e.g., the dystrophin gene). In some embodiments, the composition administered in a disclosed method of treating a subject (e.g., a human) comprises at least one (e.g., 1, 2, 3, 4, 5 or more) guide RNA. For example, in certain embodiments of a method of treatment disclosed herein, the composition comprises one dystrophin-targeted guide RNA. In some embodiments of a method of treatment disclosed herein, the composition comprises two dystrophin-targeted guide RNAs. In some embodiments of a method of treatment disclosed herein, the composition comprises more than two dystrophin-targeted guide RNAs.

A “guide RNA” or “gRNA” is a genome-targeting nucleic acid (e.g., an RNA in this context) that can direct the activities of an associated DNA endonuclease. In the context of a CRISPR/Cas or CRISPR/Cpf1 system, the endonuclease can bind to the guide RNA, which in turn, specifies the site in the target DNA to which the endonuclease is directed. CRISPR sequences in bacteria are expressed in multiple RNAs and are then processed to create guide strands for RNA. A guide RNA can comprise at least a spacer sequence that hybridizes to a target nucleic acid sequence of interest, and a CRISPR repeat sequence. In some CRISPR systems, the gRNA also comprises a second RNA called the tracrRNA sequence. In such cases the CRISPR repeat sequence and tracrRNA sequence hybridize to each other to form a duplex. The RNA duplex can then bind an endonuclease (e.g., Cas9) to form a complex. The genome-targeting nucleic acid can provide target specificity to the complex by virtue of its association with the endonuclease. The genome-targeting nucleic acid thus can direct the activity of the complexed endonuclease.

Cleavage by the CRISPR system requires complementary base pairing of the guide RNA to a 20-nucleotide DNA sequence and the requisite protospacer-adjacent motif (PAM), a short nucleotide motif found 3′ to the target site. One can, theoretically, target any unique N₂₀-PAM sequence in the genome using CRISPR technology. Currently, the most commonly used Cas9 protein is from S. pyogenes (SpCas9), which recognizes the PAM sequence NGG, and thus, the CRISPR targeting sequence is N₂₀NGG. The degenerate N in the NGG sequence, means that given a unique sequence of 20 nucleotides (N₂₀), Cas9 would cleave N₂₀AGG, N₂₀TGG, N₂₀CGG, and N₂₀GGG equally which can be an issue for precise targeting of alleles.

Exemplary Cas9 target sites within the human dystrophin gene that require the NGG PAM are provided in Table 2. Those skilled in the art would appreciate that for each case, the guide RNA directing Cas9 to the specific site is designed to be complementary to the indicated genomic target sequence.

While PAM sequences for the commonly used S. pyogenes Cas9 (NGG) are abundant throughout the human genome, they are not always positioned correctly to target particular genes for Cas9-mediated modification. Furthermore, a target sequence may have high homology elsewhere in the genome. These additional sequences (so-called “off-targets”) may be unintentionally altered during attempts to use CRISPR mediated genome engineering on the gene of interest. To circumvent this limitation, the present disclosure also contemplates use of S. pyogenes Cas9 variants with varying PAM sequence specificities. For example, the SpCas9 VQR variant exhibits a NGAN (SEQ ID NO: 5) or NGNG (SEQ ID NO: 6) PAM binding specificity; the SpCas9 EQR variant exhibits a NGAG (SEQ ID NO: 7) PAM binding specificity; and the SpCas9 VRER variant exhibits a NGCG (SEQ ID NO: 8) PAM binding specificity.

The present disclosure also contemplates the use of Cas9 homologs or other endonuclease alternatives that further broaden the range of targetable sites in the human genome due to alternative PAM binding specificities. For example, contemplated herein are S. thermophiles Cas9 that exhibits a NNAGAAW (SEQ ID NO: 9) PAM binding specificity; N. meningitides Cas9 that exhibits a NNNNGATT (SEQ ID NO: 10) PAM binding specificity; S. aureus Cas9 that exhibits a NGRRT (SEQ ID NO: 11) or NGRRN (SEQ ID NO: 12) PAM binding specificity; and T. denticola Cas9 that exhibits a NAAAAC (SEQ ID NO: 13) PAM binding specificity. Also contemplated herein is the use of the Cpf1 endonuclease which allows for targeting of genomic regions with low GC or high AT content. Unlike Cas9, Cpf1 recognizes a T-rich PAM (TTTV (SEQ ID NO: 14)), creates a staggered, double-stranded DNA cut with a 5′ overhang, and does not require a tracrRNA for function.

Nickase variants of RNA guided endonucleases, for example Cas9, can be used to increase the specificity of CRISPR-mediated genome editing. Wild type Cas9 is typically guided by a single guide RNA designed to hybridize with a specified ˜2.0 nucleotide sequence in the target sequence (such as an endogenous genomic locus). However, several mismatches can be tolerated between the guide RNA and the target locus, effectively reducing the length of required homology in the target site to, for example, as little as 13 nt of homology, and thereby resulting in elevated potential for binding and double-strand nucleic acid cleavage by the CRISPR/Cas9 complex elsewhere in the target genome—also known as off-target cleavage. Because nickase variants of Cas9 each only cut one strand, in order to create a double-strand break it is necessary for a pair of nickases to bind in close proximity and on opposite strands of the target nucleic acid, thereby creating a pair of nicks, which is the equivalent of a double-strand break. This requires that two separate guide RNAs—one for each nickase—must bind in close proximity and on opposite strands of the target nucleic acid. This requirement essentially doubles the minimum length of homology needed for the double-strand break to occur, thereby reducing the likelihood that a double-strand cleavage event will occur elsewhere in the genome, where the two guide RNA sites—if they exist—are unlikely to be sufficiently close to each other to enable the double-strand break to form.

Accordingly, in some embodiments of the methods of treatment presently disclosed, the composition comprises two guide RNAs. In some embodiments of the methods of treatment presently disclosed, the composition comprises an adeno-associated viral vector, a Cas9 nickase, and at least one (e.g., 2) guide RNA. In some embodiments of the methods of treatment presently disclosed, the composition comprises an adeno-associated viral vector, a Cas9 nickase, and two guide RNAs. In some embodiments of the methods of treatment presently disclosed, the composition comprises an adeno-associated viral vector, a Cas9 nickase, at least one (e.g., 2) guide RNA. In some embodiments, the composition may further comprise a donor DNA template. For example, in some embodiments of the methods of treatment presently disclosed, the composition comprises an adeno-associated viral vector, a Cas9 nickase, at least one (e.g., 2) guide RNA, and a donor DNA template.

Nickases can also be used to promote homology directed repair (HDR) versus the more error-prone non-homologous end joining. HDR can be used to introduce selected changes into target sites in the genome through the use of specific donor sequences that effectively mediate the desired changes. Descriptions of various CRISPR/Cas systems for use in gene editing can be found, e.g., in international patent application publication number WO2013/176772.

### AAV Vectors

While CRISPR-based targeting of disease mutations has been shown to be effective in vitro and in vivo, through mouse and other animal studies, delivery constraints have proven to be limiting. For example, there is a need for appropriate somatic cell delivery systems capable of directing the components of the CRISPR/Cas9 or CRISPR/Cpf1 systems to dystrophic muscle or satellite cells in vivo. In this regard, the non-pathogenic adeno-associated virus (AAV) delivery system has proven to be safe and effective and has already been advanced in clinical trials for gene therapy. As such, in applications where transient expression of the CRISPR components is suitable, the AAV vector based systems can be used. For example, AAV vectors are frequently used in ocular gene therapy. Several features of AAV make it an attractive choice: the virus is nonpathogenic, it infects both dividing and non-dividing cells, expression can persist for long periods of time, and it is particularly noteworthy for its history of safety, efficacy and a general lack of toxicity in clinical trials. With AAV vectors, high titer and levels of expression have been obtained. This vector can be produced in large quantities in a relatively simple system. Construction of recombinant AAV vectors are described in a number of publications, including U.S. Pat. No. 5,173,414.

The AAV genome is a 4.7 kb single-stranded DNA molecule that can be modified to carry up to 5.2 kb of recombinant DNA, although pushing this limit may lead to reduced packaging efficiency and deleted inserts. Due to the large size of the gene encoding the commonly used Cas9 protein (4.1 kb) itself, delivery with a gRNA, including promoter, terminator and viral inverted terminal repeat (ITR) sequences necessary for expression through a single viral vector, is sometimes limited by this AAV packaging capacity. Hence, at times, use of AAV vectors in gene therapy may necessitate co-transduction of multiple AAV vectors.

Techniques to produce AAV particles, in which an AAV genome to be packaged that includes the polynucleotide to be delivered, rep and cap genes, and helper virus functions are provided to a cell are standard in the art. Packaging cells are typically used to form virus particles that are capable of infecting a host cell. Such cells include 293 cells which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences.

The AAV rep and cap genes can be from any AAV serotype for which recombinant virus can be derived, and can be from a different AAV serotype than the AAV genome ITRs, including, but not limited to, AAV serotypes AAV-1, AAV-2, AAV-3, AAV-5, AAV-6, AAV-7, AAV-8, AAV-9, AAV-10, AAV-11, AAV-12, AAV-13 and AAV rh.74. AAV vector serotypes can be matched to target cell types. For example, the following exemplary cell types can be transduced by the indicated AAV serotypes among others: liver—AAV8, AAV3, AAV5, AAV9; skeletal muscle—AAV1, AAV7, AAV6, AAV8, AAV9, central nervous system—AAV5, AAV1, AAV4, photoreceptor cells—AAV5; lung—AAV9; heart—AAV8; pancreas—AAV8; and kidney AAV2. Production of pseudotyped AAV is disclosed in, for example, international patent application publication number WO01/83692.

In light of the foregoing, methods of treatment disclosed by the present invention may comprise administration of a composition comprising, at least in part, an adeno-associated viral vector (AAV), a DNA endonuclease, a guide RNA, and/or a donor DNA template. In some embodiments, the composition comprises at least one (e.g., 1, 2 or more) AAV vector. In some embodiments, the composition comprises one AAV vector. In some embodiments, the composition comprises two AAV vectors. In some embodiments, the composition comprises more than two AAV vectors.

In some embodiments, one or more of the non-AAV components of the composition are packaged into the AAV vector before administering to the subject (e.g., a human). For example, in some embodiments, the DNA endonuclease, the guide RNA(s), and/or the donor DNA template are packaged into the AAV vector. In some embodiments for example, the DNA endonuclease, the guide RNA(s), and/or the donor DNA template are packaged into one AAV vector. In other embodiments, the DNA endonuclease, the guide RNA(s), and/or the donor DNA template are packaged into multiple (e.g., 2) AAV vectors. In general, it has been found that an AAV vector can be a very effective means of delivery of a donor template, though the packaging limits for individual donors is <5 kb. As such, in some embodiments, the DNA endonuclease and guide RNA(s) are packaged into a first AAV vector, and the donor DNA template is packaged into a separate, second AAV vector. In such cases, the methods of treatment contemplated herein may comprise administration of a composition comprising both the first and second AAV vectors.

The AAV vectors contemplated by the methods of treatment of the present invention can be of any AAV serotype, such as, for example, AAV-1, AAV-2, AAV-3, AAV-4, AAV-5, AAV-6, AAV-7, AAV-8, AAV-9, AAV-10, AAV-11, AAV-12, AAV-13 and AAV rh.74. Notably, the AAV9 serotype has been shown to provide robust expression in skeletal muscle, heart and brain, the major tissues affected in DMD patients. Therefore, in some embodiments, methods of treatment disclosed by the present invention may comprise administration of a composition comprising, at least in part, an AAV9 viral vector. For example, in some embodiments, the administered composition may comprise a DNA endonuclease, at least one dystrophin-targeted guide RNA, and an AAV9 viral vector. In some embodiments, the DNA endonuclease and at least one dystrophin-targeted guide RNA are packaged into an AAV9 viral vector before administration to the subject (e.g., a human).

### EXAMPLES

### Example 1: Use of Gene Editing to Skip Exon Skipping

The present example demonstrates use of CRISPR to delete mutant or out-of-frame exons, thereby allowing splicing between surrounding exons to recreate an in-frame dystrophin protein that lacks the mutations.

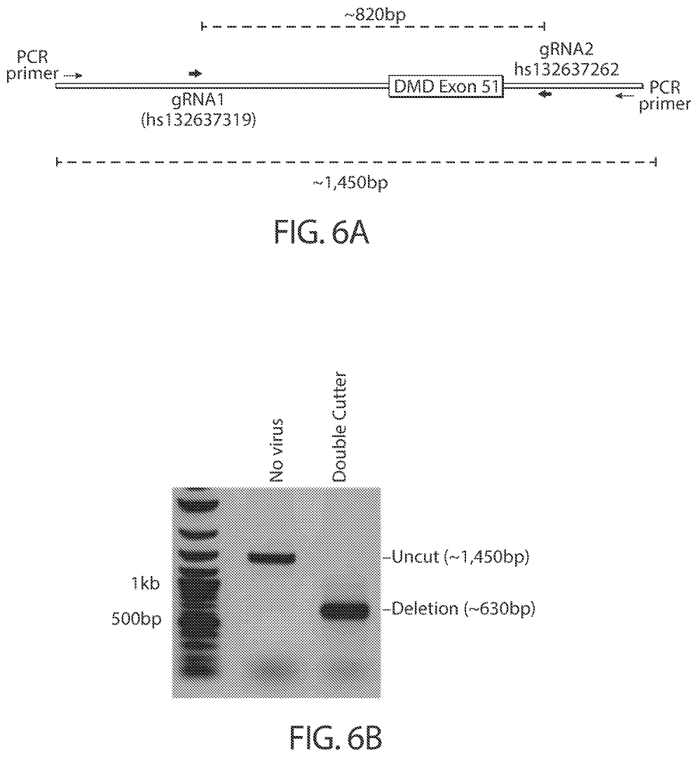

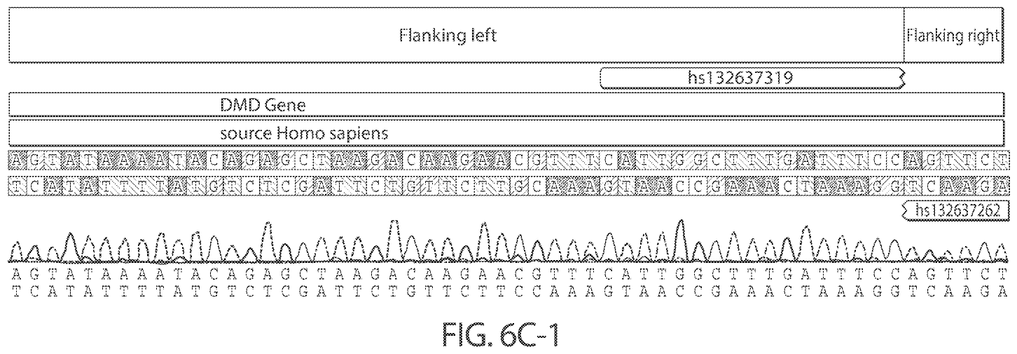

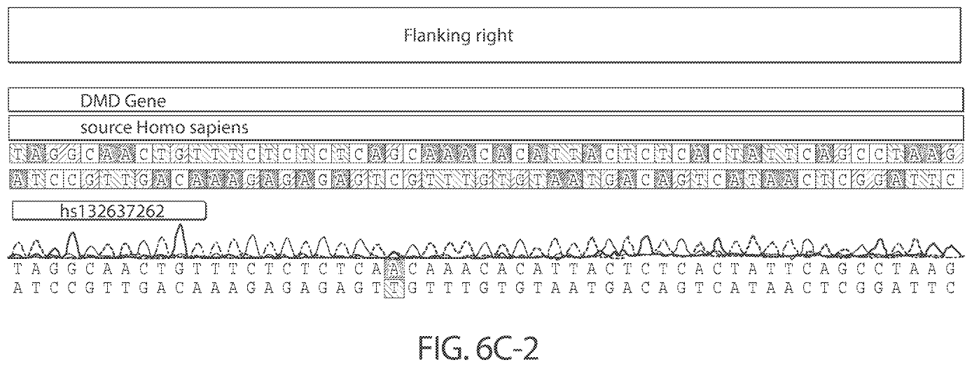

As shown in FIG. 6A, two targeting guide RNAs, hs132637319 (CATTGGCTTTGATTTCCCTAGGG (SEQ ID NO: 15)) and hs132637262 (ACAGTTGCCTAAGAACTGGTGGG (SEQ ID NO: 16)) were designed to delete exon 51 and maintain coding frame, thus bypassing frameshifting mutations on exon 51. As shown below, the entire amplicon is 1,450 bp, and the expected deletion from both guide RNAs is ˜630 bp.

Human DMD amplicon
(SEQ ID NO: 17)
GAGAGGTTATGTGGCTTTACCAAGGTCCCAGAGTTCCTAGGGCAGAGAAC
AGGATTGTCTACCAGACATTTTAATTCTAGTACTATGCATCTTAACCATT
ACCATAGGCTGACTTACTCTACAGTGTCCAACACTATTCATATTAAGATT
TATTTAATGACTTTGAAACAGTATTTCATGTCTAAATAGAAAAACTACTA
ACTCGCATTTTTAAGAAAATATTGTATCTTGGTTTTTCTTCACTGCTGGC
CAGTTTACTAACAATCTGAAATAAAAAGAAAAAAATATGATAAACTGCTC
CCAGTATAAAATACAGAGCTAAGACAAGAACGTTTCATTGGCTTTGATTT
CCCTAGGGTCCAGCTTCAAATTAATTTACTTCCTATTCAAGGGAATTTTA
AATCAGAAAGAAGATCTTATCCCATCTTGTTTTGCCTTTGTTTTTTCTTG
AATAAAAAAAAAATAAGTAAAATTTATTTCCCTGGCAAGGTCTGAAAACT
TTTGTTTTCTTTACCACTTCCACAATGTATATGATTGTTACTGAGAAGGC
TTATTTAACTTAAGTTACTTGTCCAGGCATGAGAATGAGCAAAATCGTTT
TTTAAAAAATTGTTAAATGTATATTAATGAAAAGGTTGAATCTTTTCATT
TTCTACCATGTATTGCTAAACAAAGTATCCACATTGTTAGAAAAAGATAT
ATAATGTCATGAATAAGAGTTTGGCTCAAATTGTTACTCTTCAATTAAAT
TTGACTTATTGTTATTGAAATTGGCTCTTTAGCTTGTGTTTCTAATTTTT
CTTTTTCTTCTTTTTTCCTTTTTGCAAAAACCCAAAATATTTTAGCTCCT
ACTCAGACTGTTACTCTGGTGACACAACCTGTGGTTACTAAGGAAACTGC
CATCTCCAAACTAGAAATGCCATCTTCCTTGATGTTGGAGGTACCTGCTC
TGGCAGATTTCAACCGGGCTTGGACAGAACTTACCGACTGGCTTTCTCTG
CTTGATCAAGTTATAAAATCACAGAGGGTGATGGTGGGTGACCTTGAGGA
TATCAACGAGATGATCATCAAGCAGAAGGTATGAGAAAAAATGATAAAAG
TTGGCAGAAGTTTTTCTTTAAAATGAAGATTTTCCACCAATCACTTTACT
CTCCTAGACCATTTCCCACCAGTTCTTAGGCAACTGTTTCTCTCTCAGCA
AACACATTACTCTCACTATTCAGCCTAAGTATAATCAAGGATATAAATTA
ATGCAAATAACAAAAGTAGCCATACATTAAAAAGGAAATATACAAAAAAA
AAAAAAAAAAAAAGCAGAAACCTTACAAGAATAGTTGTCTCAGTTAAATT
TACTAAACAACCTGGTATTTTAAAAATCTATTTTATACCAAATAAGTCAC
TCAACTGAGCTATTTACATTTAAACTGTTTGTTTTGGCACTACGCAG

As shown in FIG. 6B, a PCR analysis following transfection indicated that use of the CRISPR system resulted in a deleted product of the expected size. PCR primers used were:

| Human DMD exon 51 forward primer | |
|---|---|
| GAGAGGTTATGTGGCTTTACCA | (SEQ ID NO: 18) |
| Human DMD exon 51 reverse primer | |
| CTGCGTAGTGCCAAAACAAA | (SEQ ID NO: 19) |

Sequencing analysis further confirmed the deleted product following targeting (see FIG. 6C).

### Example 2: Use of Gene Editing to Skip Exon Skipping

This experiment demonstrates the ability to use CRISPR systems to cut the DMD gene at a desired location. Specifically, AAV was used to deliver to mouse N2a cells either (1) Cpf1 and a single guide RNA/crRNA (AAGGCCAAACCTCGGCTTACCTG (SEQ ID NO: 20)) (2) Cfp1 and two guide RNA/crRNA (AAAGAGCAATAAAATGGCTTCAA (SEQ ID NO: 21) and AAGGCCAAACCTCGGCTTACCTG (SEQ ID NO: 22)) or (3) MAD7 and the guide RNA/crRNA AAGGCCAAACCTCGGCTTACC (SEQ ID NO: 23) or TTTAAAGGCCAAACCTCGGCTTACC (SEQ ID NO: 24), which included a PAM sequence.

Cpf1 vector (region cloned between AAV2 ITRs)
was encoded by the following sequence:
(SEQ ID NO: 25)
GCGGCCGCGGTCCGACTAGTTAATTAAAAAAACAGGTAAGCCGAGGTTTG
GCCTTATCTACACTTAGTAGAAATTTTCAGGATGTAGACCGGCCGCCACT
ATAAGGCTCGAAAGAGGAATAAATTTTTCGTTTAGGGTGATTTCCCACAA
AGCACAGCGCGTAATTTGCATGCGCTCTACCCCAGGCTCCTGTGCTAGAC
AAGAAGCCCGCGCATCCGGGCAAGGGATGATGACGTCGTCCTTCAAGAGC
GCCGCCACCATGGCCCCCAAGAAGAAGAGGAAAGTGGGAATCCACGGCGT
GCCTGCCGCCAGCAAGCTGGAGAAGTTTACAAACTGCTACTCCCTGTCTA
AGACCCTGCGCTTCAAGGCCATCCCCGTGGGCAAGACCCAGGAGAACATC
GACAATAAGAGGCTGCTGGTGGAGGACGAGAAGAGAGCCGAGGATTATAA
GGGCGTGAAGAAGCTGCTGGATAGGTACTATCTGTCTTTTATCAACGACG
TGCTGCACAGCATCAAGCTGAAGAATCTGAACAATTACATCAGCCTGTTC
AGGAAGAAGACAAGAACCGAGAAGGAGAATAAGGAGCTGGAGAACCTGGA
GATCAATCTGAGAAAGGAGATCGCCAAGGCCTTCAAGGGCAACGAGGGCT
ACAAGTCCCTGTTTAAGAAGGATATCATCGAGACAATCCTGCCTGAGTTC
CTGGACGATAAGGACGAGATCGCCCTGGTGAACAGCTTCAATGGCTTTAC
CACAGCCTTCACCGGCTTCTTTGATAACAGAGAGAATATGTTTTCCGAGG
AGGCCAAGAGCACATCCATCGCCTTCCGGTGTATCAACGAGAATCTGACC
CGCTACATCTCTAATATGGACATCTTCGAGAAGGTGGACGCCATCTTTGA
TAAGCACGAGGTGCAGGAGATCAAGGAGAAGATCCTGAACAGCGACTATG
ATGTGGAGGATTTCTTTGAGGGCGAGTTCTTTAACTTTGTGCTGACACAG
GAGGGCATCGACGTGTACAATGCCATCATCGGCGGCTTCGTGACCGAGAG
CGGCGAGAAGATCAAGGGCCTGAACGAGTACATCAACCTGTATAATCAGA
AGACCAAGCAGAAGCTGCCTAAGTTTAAGCCACTGTACAAGCAGGTGCTG
AGCGATAGGGAGTCTCTGAGCTTCTACGGCGAGGGCTATACATCCGATGA
GGAGGTGCTGGAGGTGTTTAGAAACACCCTGAACAAGAATTCCGAGATCT
TCAGCTCCATCAAGAAGCTGGAGAAGCTGTTCAAGAATTTTGACGAGTAC
TCTAGCGCCGGCATCTTTGTGAAGAACGGCCCTGCCATCAGCACAATCTC
CAAGGATATCTTCGGCGAGTGGAACGTGATCCGGGACAAGTGGAATGCCG
AGTATGACGATATCCACCTGAAGAAGAAGGCCGTGGTGACCGAGAAGTAC
GAGGACGATAGGAGAAAGTCCTTCAAGAAGATCGGCTCCTTTTCTCTGGA
GCAGCTGCAGGAGTACGCCGACGCCGATCTGTCTGTGGTGGAGAAGCTGA
AGGAGATCATCATCCAGAAGGTGGATGAGATCTACAAGGTGTATGGCTCC
TCTGAGAAGCTGTTCGACGCCGATTTTGTGCTGGAGAAGAGCCTGAAGAA
GAACGACGCCGTGGTGGCCATCATGAAGGACCTGCTGGATTCTGTGAAGA
GCTTCGAGAATTACATCAAGGCCTTCTTTGGCGAGGGCAAGGAGACAAAC
CGGGACGAGTCCTTCTATGGCGATTTTGTGCTGGCCTACGACATCCTGCT
GAAGGTGGACCACATCTACGATGCCATCCGCAATTATGTGACCCAGAAGC
CATACTCTAAGGATAAGTTCAAGCTGTATTTTCAGAACCCCCAGTTCATG
GGCGGCTGGGACAAGGATAAGGAGACAGACTATAGGGCCACCATCCTGAG
ATACGGCTCCAAGTACTATCTGGCCATCATGGATAAGAAGTACGCCAAGT
GCCTGCAGAAGATCGACAAGGACGATGTGAACGGCAATTACGAGAAGATC
AACTATAAGCTGCTGCCCGGCCCTAATAAGATGCTGCCTAAGGTGTTCTT
TTCTAAGAAGTGGATGGCCTACTATAACCCAAGCGAGGACATCCAGAAGA
TCTACAAGAATGGCACATTCAAGAAGGGCGATATGTTTAACCTGAATGAC
TGTCACAAGCTGATCGACTTCTTTAAGGATAGCATCTCCAGGTATCCTAA
GTGGTCCAATGCCTACGATTTCAACTTTTCTGAGACAGAGAAGTATAAGG
ACATCGCCGGCTTTTACAGAGAGGTGGAGGAGCAGGGCTATAAGGTGAGC
TTCGAGTCTGCCAGCAAGAAGGAGGTGGATAAGCTGGTGGAGGAGGGCAA
GCTGTACATGTTCCAGATCTATAACAAGGACTTTTCCGATAAGTCTCACG
GCACACCAAATCTGCACACCATGTACTTCAAGCTGCTGTTTGACGAGAAC
AATCACGGCCAGATCAGGCTGAGCGGAGGAGCAGAGCTGTTCATGAGGAG
GGCCTCCCTGAAGAAGGAGGAGCTGGTGGTGCACCCAGCAAACTCCCCTA
TCGCCAACAAGAATCCAGATAATCCCAAGAAGACCACAACCCTGTCCTAC
GACGTGTATAAGGATAAGAGGTTTTCTGAGGACCAGTACGAGCTGCACAT
CCCAATCGCCATCAATAAGTGCCCCAAGAACATCTTCAAGATCAATACAG
AGGTGAGAGTGCTGCTGAAGCACGACGATAACCCATATGTGATCGGCATC
GATCGGGGCGAGCGCAATCTGCTGTACATCGTGGTGGTGGACGGCAAGGG
CAACATCGTGGAGCAGTATTCCCTGAACGAGATCATCAACAACTTCAACG
GCATCCGGATCAAGACAGATTACCACTCTCTGCTGGACAAGAAGGAGAAG
GAGCGGTTCGAGGCCCGCCAGAACTGGACCTCCATCGAGAATATCAAGGA
GCTGAAGGCCGGCTATATCTCTCAGGTGGTGCACAAGATCTGCGAGCTGG
TGGAGAAGTACGATGCCGTGATCGCCCTGGAGGACCTGAACTCTGGCTTT
AAGAATAGCCGCGTGAAGGTGGAGAAGCAGGTGTATCAGAAGTTCGAGAA
GATGCTGATCGATAAGCTGAACTACATGGTGGACAAGAAGTCTAATCCAT
GTGCAACAGGCGGCGCCCTGAAGGGATATCAGATCACCAATAAGTTCGAG
AGCTTTAAGTCCATGTCTACCCAGAACGGCTTCATCTTTTACATCCCTGC
CTGGCTGACATCCAAGATCGACCCATCTACCGGCTTCGTGAACCTGCTGA
AGACAAAGTATACCAGCATCGCCGATTCCAAGAAGTTCATCAGCTCCTTT
GACCGGATCATGTACGTGCCAGAGGAGGATCTGTTCGAGTTTGCCCTGGA
CTATAAGAACTTCTCTCGCACAGACGCCGATTACATCAAGAAGTGGAAGC
TGTACAGCTATGGCAACAGGATCAGAATCTTCCGGAATCCCAAGAAGAAC
AACGTGTTCGACTGGGAGGAGGTGTGCCTGACCAGCGCCTATAAGGAGCT
GTTCAACAAGTACGGCATCAATTATCAGCAGGGCGATATCCGCGCCCTGC
TGTGCGAGCAGTCCGACAAGGCCTTCTACTCTAGCTTTATGGCCCTGATG
AGCCTGATGCTGCAGATGCGGAATTCCATCACAGGCCGCACCGACGTGGA
TTTTCTGATCAGCCCCGTGAAGAACTCCGACGGCATCTTCTACGATAGCA
GGAACTATGAGGCCCAGGAGAATGCCATCCTGCCTAAGAACGCCGACGCC
AATGGCGCCTATAACATCGCCAGAAAGGTGCTGTGGGCCATCGGCCAGTT
CAAGAAGGCCGAGGACGAGAAGCTGGATAAGGTGAAGATCGCCATCTCTA
ACAAGGAGTGGCTGGAGTACGCCCAGACCAGCGTGAAGCATAAACGACCA
GCCGCAACTAAAAAGGCAGGGCAGGCAAAGAAGAAGAAAGGAAGCTAACC
GGTCAGACATGATAAGATACATTGATGAGTTTGGACAAACCACAACTAGA
ATGCAGTGAAAAAAATGCTTTATTTGTGAAATTTGTGATGCTATTGCTTT
ATTTGTAACCATTATAAGCTGCAATAAACAAGTTAACAACAACAATTGCA
TTCATTTTATGTTTCAGGTTCAGGGGGAGGTGTGGGAGGTTTTTTAAAGC
TAGCGGCCGC
Cpf1 dual vector (region cloned between AAV2 ITRs)
was encoded by the following sequence:
(SEQ ID NO: 26)
GCGGCCGCGGTCCGACTAGTTAATTAAAAAAACAGGTAAGCCGAGGTTTG
GCCTTATCTACACTTAGTAGAAATTTTGAAGCCATTTTATTGCTCTTTAT
CTACACTTAGTAGAAATTTTCAGGATGTAGACCGGCCGCCACTATAAGGC
TCGAAAGAGGAATAAATTTTTCGTTTAGGGTGATTTCCCACAAAGCACAG
CGCGTAATTTGCATGCGCTCTACCCCAGGCTCCTGTGCTAGACAAGAAGC
CCGCGCATCCGGGCAAGGGATGATGACGTCGTCCTTCAAGAGCGCCGCCA
CCATGGCCCCCAAGAAGAAGAGGAAAGTGGGAATCCACGGCGTGCCTGCC
GCCAGCAAGCTGGAGAAGTTTACAAACTGCTACTCCCTGTCTAAGACCCT
GCGCTTCAAGGCCATCCCCGTGGGCAAGACCCAGGAGAACATCGACAATA
AGAGGCTGCTGGTGGAGGACGAGAAGAGAGCCGAGGATTATAAGGGCGTG
AAGAAGCTGCTGGATAGGTACTATCTGTCTTTTATCAACGACGTGCTGCA
CAGCATCAAGCTGAAGAATCTGAACAATTACATCAGCCTGTTCAGGAAGA
AGACAAGAACCGAGAAGGAGAATAAGGAGCTGGAGAACCTGGAGATCAAT
CTGAGAAAGGAGATCGCCAAGGCCTTCAAGGGCAACGAGGGCTACAAGTC
CCTGTTTAAGAAGGATATCATCGAGACAATCCTGCCTGAGTTCCTGGACG
ATAAGGACGAGATCGCCCTGGTGAACAGCTTCAATGGCTTTACCACAGCC
TTCACCGGCTTCTTTGATAACAGAGAGAATATGTTTTCCGAGGAGGCCAA
GAGCACATCCATCGCCTTCCGGTGTATCAACGAGAATCTGACCCGCTACA
TCTCTAATATGGACATCTTCGAGAAGGTGGACGCCATCTTTGATAAGCAC
GAGGTGCAGGAGATCAAGGAGAAGATCCTGAACAGCGACTATGATGTGGA
GGATTTCTTTGAGGGCGAGTTCTTTAACTTTGTGCTGACACAGGAGGGCA
TCGACGTGTACAATGCCATCATCGGCGGCTTCGTGACCGAGAGCGGCGAG
AAGATCAAGGGCCTGAACGAGTACATCAACCTGTATAATCAGAAGACCAA
GCAGAAGCTGCCTAAGTTTAAGCCACTGTACAAGCAGGTGCTGAGCGATA
GGGAGTCTCTGAGCTTCTACGGCGAGGGCTATACATCCGATGAGGAGGTG
CTGGAGGTGTTTAGAAACACCCTGAACAAGAATTCCGAGATCTTCAGCTC
CATCAAGAAGCTGGAGAAGCTGTTCAAGAATTTTGACGAGTACTCTAGCG
CCGGCATCTTTGTGAAGAACGGCCCTGCCATCAGCACAATCTCCAAGGAT
ATCTTCGGCGAGTGGAACGTGATCCGGGACAAGTGGAATGCCGAGTATGA
CGATATCCACCTGAAGAAGAAGGCCGTGGTGACCGAGAAGTACGAGGACG
ATAGGAGAAAGTCCTTCAAGAAGATCGGCTCCTTTTCTCTGGAGCAGCTG
CAGGAGTACGCCGACGCCGATCTGTCTGTGGTGGAGAAGCTGAAGGAGAT
CATCATCCAGAAGGTGGATGAGATCTACAAGGTGTATGGCTCCTCTGAGA
AGCTGTTCGACGCCGATTTTGTGCTGGAGAAGAGCCTGAAGAAGAACGAC
GCCGTGGTGGCCATCATGAAGGACCTGCTGGATTCTGTGAAGAGCTTCGA
GAATTACATCAAGGCCTTCTTTGGCGAGGGCAAGGAGACAAACCGGGACG
AGTCCTTCTATGGCGATTTTGTGCTGGCCTACGACATCCTGCTGAAGGTG
GACCACATCTACGATGCCATCCGCAATTATGTGACCCAGAAGCCATACTC
TAAGGATAAGTTCAAGCTGTATTTTCAGAACCCCCAGTTCATGGGCGGCT
GGGACAAGGATAAGGAGACAGACTATAGGGCCACCATCCTGAGATACGGC
TCCAAGTACTATCTGGCCATCATGGATAAGAAGTACGCCAAGTGCCTGCA
GAAGATCGACAAGGACGATGTGAACGGCAATTACGAGAAGATCAACTATA
AGCTGCTGCCCGGCCCTAATAAGATGCTGCCTAAGGTGTTCTTTTCTAAG
AAGTGGATGGCCTACTATAACCCAAGCGAGGACATCCAGAAGATCTACAA
GAATGGCACATTCAAGAAGGGCGATATGTTTAACCTGAATGACTGTCACA
AGCTGATCGACTTCTTTAAGGATAGCATCTCCAGGTATCCTAAGTGGTCC
AATGCCTACGATTTCAACTTTTCTGAGACAGAGAAGTATAAGGACATCGC
CGGCTTTTACAGAGAGGTGGAGGAGCAGGGCTATAAGGTGAGCTTCGAGT
CTGCCAGCAAGAAGGAGGTGGATAAGCTGGTGGAGGAGGGCAAGCTGTAC
ATGTTCCAGATCTATAACAAGGACTTTTCCGATAAGTCTCACGGCACACC
AAATCTGCACACCATGTACTTCAAGCTGCTGTTTGACGAGAACAATCACG
GCCAGATCAGGCTGAGCGGAGGAGCAGAGCTGTTCATGAGGAGGGCCTCC
CTGAAGAAGGAGGAGCTGGTGGTGCACCCAGCAAACTCCCCTATCGCCAA
CAAGAATCCAGATAATCCCAAGAAGACCACAACCCTGTCCTACGACGTGT
ATAAGGATAAGAGGTTTTCTGAGGACCAGTACGAGCTGCACATCCCAATC
GCCATCAATAAGTGCCCCAAGAACATCTTCAAGATCAATACAGAGGTGAG
AGTGCTGCTGAAGCACGACGATAACCCATATGTGATCGGCATCGATCGGG
GCGAGCGCAATCTGCTGTACATCGTGGTGGTGGACGGCAAGGGCAACATC
GTGGAGCAGTATTCCCTGAACGAGATCATCAACAACTTCAACGGCATCCG
GATCAAGACAGATTACCACTCTCTGCTGGACAAGAAGGAGAAGGAGCGGT
TCGAGGCCCGCCAGAACTGGACCTCCATCGAGAATATCAAGGAGCTGAAG
GCCGGCTATATCTCTCAGGTGGTGCACAAGATCTGCGAGCTGGTGGAGAA
GTACGATGCCGTGATCGCCCTGGAGGACCTGAACTCTGGCTTTAAGAATA
GCCGCGTGAAGGTGGAGAAGCAGGTGTATCAGAAGTTCGAGAAGATGCTG
ATCGATAAGCTGAACTACATGGTGGACAAGAAGTCTAATCCATGTGCAAC
AGGCGGCGCCCTGAAGGGATATCAGATCACCAATAAGTTCGAGAGCTTTA
AGTCCATGTCTACCCAGAACGGCTTCATCTTTTACATCCCTGCCTGGCTG
ACATCCAAGATCGACCCATCTACCGGCTTCGTGAACCTGCTGAAGACAAA
GTATACCAGCATCGCCGATTCCAAGAAGTTCATCAGCTCCTTTGACCGGA
TCATGTACGTGCCAGAGGAGGATCTGTTCGAGTTTGCCCTGGACTATAAG
AACTTCTCTCGCACAGACGCCGATTACATCAAGAAGTGGAAGCTGTACAG
CTATGGCAACAGGATCAGAATCTTCCGGAATCCCAAGAAGAACAACGTGT
TCGACTGGGAGGAGGTGTGCCTGACCAGCGCCTATAAGGAGCTGTTCAAC
AAGTACGGCATCAATTATCAGCAGGGCGATATCCGCGCCCTGCTGTGCGA
GCAGTCCGACAAGGCCTTCTACTCTAGCTTTATGGCCCTGATGAGCCTGA
TGCTGCAGATGCGGAATTCCATCACAGGCCGCACCGACGTGGATTTTCTG
ATCAGCCCCGTGAAGAACTCCGACGGCATCTTCTACGATAGCAGGAACTA
TGAGGCCCAGGAGAATGCCATCCTGCCTAAGAACGCCGACGCCAATGGCG
CCTATAACATCGCCAGAAAGGTGCTGTGGGCCATCGGCCAGTTCAAGAAG
GCCGAGGACGAGAAGCTGGATAAGGTGAAGATCGCCATCTCTAACAAGGA
GTGGCTGGAGTACGCCCAGACCAGCGTGAAGCATAAACGACCAGCCGCAA
CTAAAAAGGCAGGGCAGGCAAAGAAGAAGAAAGGAAGCTAACCGGTCAGA
CATGATAAGATACATTGATGAGTTTGGACAAACCACAACTAGAATGCAGT
GAAAAAAATGCTTTATTTGTGAAATTTGTGATGCTATTGCTTTATTTGTA
ACCATTATAAGCTGCAATAAACAAGTTAACAACAACAATTGCATTCATTT
TATGTTTCAGGTTCAGGGGGAGGTGTGGGAGGTTTTTTAAAGCTAGCGGC
CGC

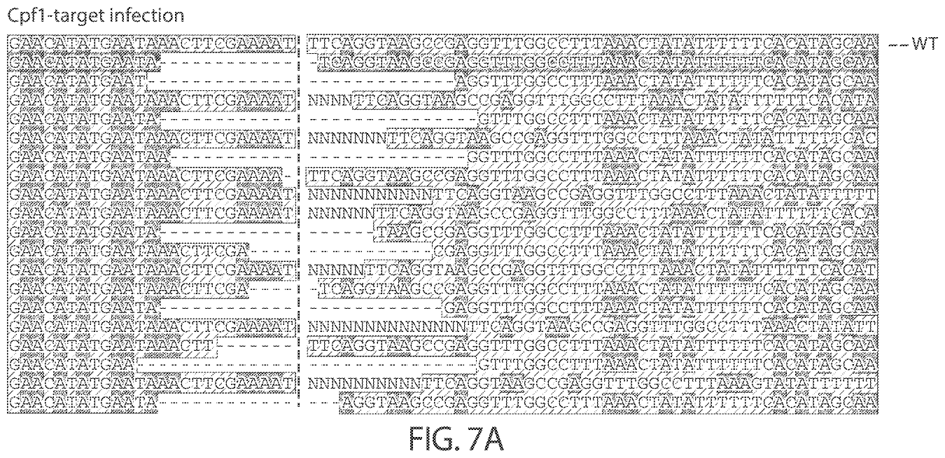

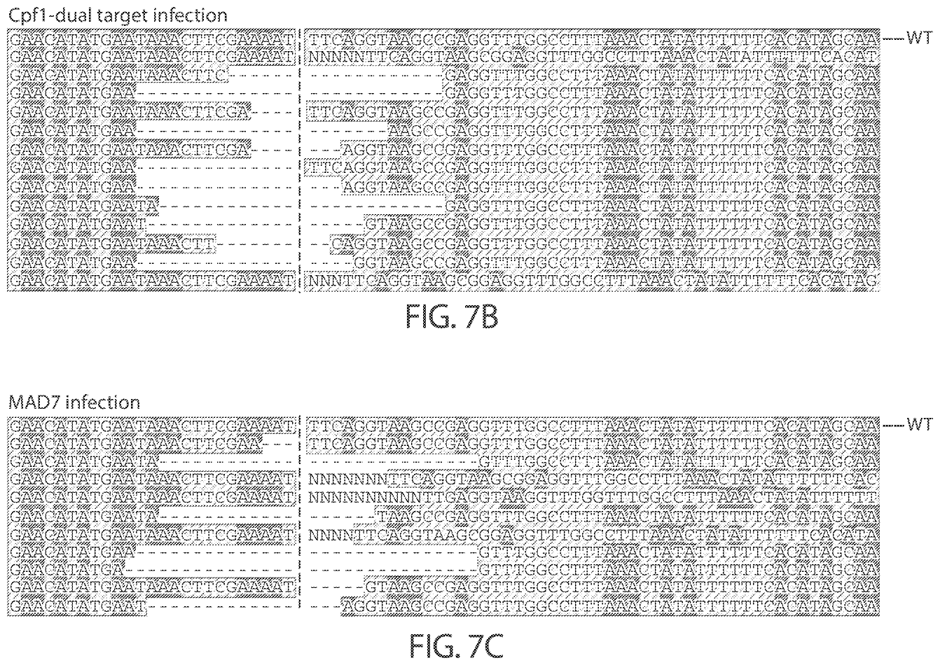

As shown in FIGS. 7A-C, an Interference of CRISPR Edits (ICE) analysis was performed indicating cutting of the mouse DMD gene and mutations around the expected site (dotted line) in exon 23 following infection for all three experiments. A discussion of ICE analysis is found at www.synthego.com.

MAD7 vector (region cloned between AAV2 ITRs)
was encoded by the following sequence:
(SEQ ID NO: 28)
GCGGCCGCGGTCCGACTAGTTAATTAAAAAAAGGTAAGCCGAGGTTTGGC
CTTATCTACAAGAGTAGAAATTAAAAAGGTCTTTTGACTTCAGGATGTAG
ACCGGCCGCCACTATAAGGCTCGAAAGAGGAATAAATTTTTCGTTTAGGG
TGATTTCCCACAAAGCACAGCGCGTAATTTGCATGCGCTCTACCCCAGGC
TCCTGTGCTAGACAAGAAGCCCGCGCATCCGGGCAAGGGATGATGACGTC
GTCCTTCAAGAGCGCCGCCACCATGGCACCCAAGAAGAAAAGAAAGGTCG
GAATCCACGGAGTCCCAGCAGCTAACAATGGAACTAACAACTTCCAGAAC
TTCATCGGGATCAGCTCCCTGCAGAAGACCCTGAGGAACGCCCTGATCCC
CACAGAGACAACACAGCAGTTCATCGTGAAGAATGGCATCATCAAGGAGG
ATGAGCTGAGGGGCGAGAACAGACAGATCCTGAAGGACATCATGGACGAT
TACTATAGAGGCTTCATCAGCGAGACACTGTCTAGCATCGACGATATCGA
CTGGACATCCCTGTTTGAGAAGATGGAGATCCAGCTGAAGAATGGCGATA
ACAAGGACACCCTGATCAAGGAGCAGACAGAGTACAGGAAGGCCATCCAC
AAGAAGTTCGCCAATGACGATAGATTCAAGAACATGTTTTCCGCCAAGCT
GATCTCTGACATCCTGCCTGAGTTTGTGATCCACAACAATAACTATAGCG
CCTCCGAGAAGGAGGAGAAGACCCAGGTCATCAAGCTGTTCAGCCGGTTT
GCCACATCCTTCAAGGACTACTTTAAGAATCGCGCCAACTGCTTCAGCGC
CGACGATATCTCCTCTAGCTCCTGTCACCGGATCGTGAATGATAACGCCG
AGATCTTCTTTTCCAACGCCCTGGTGTACAGGAGAATCGTGAAGTCTCTG
AGCAATGACGATATCAACAAGATCTCTGGCGATATGAAGGACTCTCTGAA
GGAGATGAGCCTGGAGGAGATCTACAGCTATGAGAAGTATGGCGAGTTCA
TCACCCAGGAGGGCATCTCTTTTTACAACGACATCTGCGGCAAGGTCAAT
AGCTTCATGAACCTGTATTGTCAGAAGAATAAGGAGAATAAGAACCTGTA
CAAGCTGCAGAAGCTGCACAAGCAGATCCTGTGCATCGCCGATACAAGCT
ATGAGGTGCCATACAAGTTCGAGTCTGACGAGGAGGTGTACCAGAGCGTG
AATGGCTTTCTGGATAACATCTCTAGCAAGCACATCGTGGAGAGGCTGAG
AAAGATCGGCGATAATTATAACGGCTACAACCTGGACAAGATCTACATCG
TGTCCAAGTTCTATGAGTCCGTGTCTCAGAAGACCTATCGCGACTGGGAG
ACAATCAACACAGCCCTGGAGATCCACTACAATAACATCCTGCCCGGCAA
CGGCAAGAGCAAGGCCGATAAGGTGAAGAAGGCCGTGAAGAATGACCTGC
AGAAGTCCATCACCGAGATCAATGAGCTGGTGAGCAACTATAAGCTGTGC
TCCGACGATAACATCAAGGCCGAGACATACATCCACGAGATCAGCCACAT
CCTGAATAACTTCGAGGCCCAGGAGCTGAAGTATAATCCCGAGATCCACC
TGGTGGAGTCCGAGCTGAAGGCCTCTGAGCTGAAGAATGTGCTGGACGTG
ATCATGAACGCCTTCCACTGGTGTTCCGTGTTTATGACCGAGGAGCTGGT
GGACAAGGATAATAACTTTTACGCCGAGCTGGAGGAGATCTATGATGAGA
TCTACCCTGTGATCTCCCTGTACAATCTGGTGCGGAACTATGTGACCCAG
AAGCCTTACTCTACAAAGAAGATCAAGCTGAACTTCGGAATCCCAACCCT
GGCCGACGGATGGAGCAAGTCCAAGGAGTACTCTAATAACGCCATCATCC
TGATGCGCGATAATCTGTACTATCTGGGCATCTTTAATGCCAAGAACAAG
CCTGACAAGAAGATCATCGAGGGCAATACAAGCGAGAACAAGGGCGATTA
TAAGAAGATGATCTACAATCTGCTGCCCGGCCCTAACAAGATGATCCCCA
AGGTGTTCCTGTCCTCTAAGACCGGCGTGGAGACATATAAGCCTAGCGCC
TACATCCTGGAGGGCTATAAGCAGAACAAGCACATCAAGAGCTCCAAGGA
CTTCGATATCACCTTTTGCCACGATCTGATCGACTACTTCAAGAATTGTA
TCGCCATCCACCCAGAGTGGAAGAACTTCGGCTTTGATTTCTCCGACACC
TCTACATATGAGGACATCTCCGGCTTTTACAGGGAGGTGGAGCTGCAGGG
CTATAAGATCGATTGGACATACATCTCTGAGAAGGACATCGATCTGCTGC
AGGAGAAGGGCCAGCTGTACCTGTTCCAGATCTATAACAAGGACTTCAGC
AAGAAGAGCACCGGCAATGACAACCTGCACACAATGTACCTGAAGAATCT
GTTCTCCGAGGAGAACCTGAAGGACATCGTGCTGAAGCTGAATGGCGAGG
CCGAGATCTTCTTTAGAAAGTCTAGCATCAAGAATCCTATCATCCACAAG
AAGGGCTCTATCCTGGTGAACAGGACCTATGAGGCCGAGGAGAAGGACCA
GTTCGGCAACATCCAGATCGTGAGAAAGAATATCCCAGAGAACATCTACC
AGGAGCTGTACAAGTACTTCAACGATAAGAGCGACAAGGAGCTGTCCGAT
GAGGCCGCCAAGCTGAAGAATGTGGTGGGACACCACGAGGCAGCAACCAA
CATCGTGAAGGATTACAGGTACACATACGACAAGTACTTCCTGCACATGC
CAATCACCATCAATTTCAAGGCCAACAAGACAGGCTTTATCAACGACCGC
ATCCTGCAGTATATCGCCAAGGAGAAGGATCTGCACGTGATCGGAATCGA
CCGGGGCGAGCGCAATCTGATCTACGTGTCTGTGATCGACACCTGTGGCA
ACATCGTGGAGCAGAAGAGCTTTAATATCGTGAACGGCTATGATTACCAG
ATCAAGCTGAAGCAGCAGGAGGGAGCAAGGCAGATCGCACGCAAGGAGTG
GAAGGAGATCGGCAAGATCAAGGAGATCAAGGAGGGCTACCTGAGCCTGG
TCATCCACGAGATCTCTAAGATGGTCATCAAGTATAACGCCATCATCGCT
ATGGAGGACCTGAGCTATGGCTTCAAGAAGGGCCGGTTTAAGGTGGAGCG
CCAGGTGTATCAGAAGTTCGAGACAATGCTGATCAATAAGCTGAACTACC
TGGTGTTTAAGGACATCTCCATCACCGAGAACGGCGGCCTGCTGAAGGGC
TATCAGCTGACATACATCCCTGATAAGCTGAAGAATGTGGGCCACCAGTG
CGGCTGTATCTTCTACGTGCCAGCCGCCTATACCTCCAAGATCGACCCCA
CCACAGGCTTTGTGAACATCTTTAAGTTCAAGGATCTGACAGTGGACGCC
AAGCGGGAGTTCATCAAGAAGTTTGATTCTATCCGCTATGACAGCGAGAA
GAACCTGTTTTGCTTCACCTTTGATTACAATAACTTCATCACCCAGAATA
CAGTGATGTCCAAGTCCTCTTGGAGCGTGTACACATACGGCGTGAGGATC
AAGCGGCGCTTCGTGAATGGCAGATTTTCTAACGAGAGCGATACCATCGA
CATCACAAAGGATATGGAGAAGACCCTGGAGATGACAGACATCAACTGGA
GGGATGGCCACGACCTGAGACAGGATATCATCGACTACGAGATCGTGCAG
CACATCTTCGAGATCTTTCGGCTGACCGTGCAGATGCGCAACTCTCTGAG
CGAGCTGGAGGACAGGGATTATGACAGACTGATCTCCCCAGTGCTGAATG
AGAATAACATCTTCTACGATTCTGCCAAGGCAGGCGACGCACTGCCAAAG
GATGCAGACGCAAACGGCGCCTATTGTATCGCCCTGAAGGGCCTGTACGA
GATCAAGCAGATCACAGAGAACTGGAAGGAGGATGGCAAGTTTAGCAGAG
ACAAGCTGAAGATCTCCAATAAGGACTGGTTCGACTTTATTCAGAACAAG
AGGTATCTGCCTAAGAAGAAGAGGAAGGTGTAACCGGTCAGACATGATAA
GATACATTGATGAGTTTGGACAAACCACAACTAGAATGCAGTGAAAAAAA
TGCTTTATTTGTGAAATTTGTGATGCTATTGCTTTATTTGTAACCATTAT
AAGCTGCAATAAACAAGTTAACAACAACAATTGCATTCATTTTATGTTTC
AGGTTCAGGGGGAGGTGTGGGAGGTTTTTTAAAGCTAGCGGCCGC

### EQUIVALENTS

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein, will become apparent to those skilled in the art from the foregoing description and accompanying drawings using no more than routine experimentation. Such modifications and equivalents are intended to fall within the scope of the appended claims.

### INCORPORATION BY REFERENCE

The entire disclosure of each of the patent and scientific documents referred to herein is incorporated by reference for all purposes. The reference van Deutekom J, van Ommen G. Nat Rev Genet. 2003 October; 4(10):774-83 is incorporated herein by reference for all purposes.

TABLE 2
Dystrophin gene intronic target sites

| | | SEQ |
|---|---|---|
| | | ID |
| grID | sequence | NO. |
| hs132692864 | TATGACAAGAGACATCCATGGGG | 29 |
| hs132693671 | AAAATGTGTAAGTAGCACTGTGG | 30 |
| hs132688560 | ATACTTATATTCAAGCACAGGGG | 31 |
| hs132693865 | TAGGAAGATTCTATGCACAGGGG | 32 |
| hs132685714 | AAAGAACCTAAAGTACACAGGGG | 33 |
| hs132684395 | CATGTAGATTAGCCTCACAGGGG | 34 |
| hs132685328 | ATTGAGATTAGAGCAACACGGGG | 35 |
| hs132689934 | TGCAACTAAATATCACACTGTGG | 36 |
| hs132691849 | TCAGATAGTCTACTACCACGTGG | 37 |
| hs132689886 | TGTTATGAACAGATGTTACGTGG | 38 |
| hs132686696 | CGGGTGGGGAAAGAAATATGCGG | 39 |
| hs132690456 | TCCAAACCAGAATAGCACAGTGG | 40 |
| hs132692865 | ATGACAAGAGACATCCATGGGGG | 41 |
| hs132684946 | ACACCCAGATTAAGGCATCGGGG | 42 |
| hs132685812 | GGATCAGGAAGGTCTCACTGTGG | 43 |
| hs132684886 | GAAGATGTCAGGGAACCCTGGGG | 44 |
| hs132687410 | AAGACTATTACAATAAAACGGGG | 45 |
| hs132687348 | AAGATCTGGGAACAACTACGTGG | 46 |
| hs132686482 | ACAGGAATCTGGGAACACTGAGG | 47 |
| hs132687291 | TTTCTACAATCTCATCACTGTGG | 48 |
| hs132692095 | GACCACCCTAAAAATCACCAGGG | 49 |
| hs132686763 | ATGCTTTCTCTAAAACACAGAGG | 50 |
| hs132693151 | TCAATTAGTACAAAGCCATGGGG | 51 |
| hs132687127 | TATCGTACCGATGAACCCTGAGG | 52 |
| hs132683622 | AAATAGACACGATTGCTACGTGG | 53 |
| hs132693659 | GTTTCATGAATAAAGCCCAGAGG | 54 |
| hs132684410 | ACAGACACCCCAAATCCTGGAGG | 55 |
| hs132688945 | GGGTGATTTCAAAGCCACAGAGG | 56 |
| hs132688960 | GCACTTGGCTGGTAATACAGGGG | 57 |
| hs132683504 | TTAGTGGTCAGCTTACAATGAGG | 58 |
| hs132687589 | CTTGACTCAATTCTCCTGAGCGG | 59 |
| hs132693827 | CTTACACATGACCTCCCCAGAGG | 60 |
| hs132692747 | AATCTTAGGAGTAATTACCACGG | 61 |
| hs132693213 | TTGCATTCCAAGAATCACAGTGG | 62 |
| hs132686932 | GTCATCCAAATATATCCCAGGGG | 63 |
| hs132692495 | TGTAACTGGAGACAAGAAGGGGG | 64 |
| hs132690588 | TCAACCAATATGGATCACTGTGG | 65 |
| hs132686172 | CCAAACTGATAAGTCCTCAGGGG | 66 |
| hs132690946 | AATTGTCCAGTAAATCCATGTGG | 67 |
| hs132690397 | ACTGTATAGGAACTACATGGAGG | 68 |
| hs132687602 | AATCATACTGAATATCACTGAGG | 69 |
| hs132683437 | TTTATTTTATTCTCTCTACGGGG | 70 |
| hs132686632 | ACTGGGAAGCTCTTCCACAGTGG | 71 |
| hs132692204 | TAGGGAGGAATCCCACAATGGGG | 72 |
| hs132691458 | AATCCCCTACATTCTCAGGGAGG | 73 |
| hs132688992 | GAAGTGCTTGAAGAGGAACAGGG | 74 |
| hs132688164 | TAAATGAGAGAAGCCCATGGTGG | 75 |
| hs132685798 | CATCATCTCCTACTTCACTGGGG | 76 |
| hs132689462 | CAGACTCTTAAGCAGCAGTGGGG | 77 |
| hs132692750 | GAATGAAAACAAACCCACCGTGG | 78 |
| hs132692262 | CAGACATTCAAAATTCTGGGTGG | 79 |
| hs132688865 | TCTTCTGTGACTATGCAGGGTGG | 80 |
| hs132692640 | AAAGTGAAAGAGCCTCCTCGAGG | 81 |
| hs132688568 | GAGATAAAGAGAAAGATTGGGGG | 82 |
| hs132689902 | GTGTAAAGAACACTACAACACGG | 83 |
| hs132684145 | TGACTAAACACTGAGCCATGAGG | 84 |
| hs132683758 | TCTATTGGCAATGTACACTGAGG | 85 |
| hs132684428 | CAGAGATGTGACATCCAATGTGG | 86 |
| hs132690386 | ATAATAGACATGAAACAACAAGG | 87 |
| hs132687217 | ATAAGCGGGTAGCAGTCCTGCGG | 88 |
| hs132689080 | ATAAATATGATGATCCCAGGAGG | 89 |
| hs132690427 | CTACAGGACTCAGATCTGGGAGG | 90 |
| hs132685282 | TCTAGGCTAGAGTAAAGCTGTGG | 91 |
| hs132692268 | AATTGCGTATGGCTTCATGTGGG | 92 |
| hs132684093 | GATCAAGATCAAGACCGGTGTGG | 93 |
| hs132691237 | TCACCACAGCAACCCCAGGGAGG | 94 |
| hs132684573 | AGTATGTGACGTCAATTGAGTGG | 95 |
| hs132689458 | AGCAGTCCTCAGTAACTCAGGGG | 96 |
| hs132690552 | ACTCTGGGACATGAATCTAGAGG | 97 |
| hs132687556 | CTGTTACCAGTACAGTACAGTGG | 98 |
| hs132684205 | CCTGGGAAGTCAATGTACAGTGG | 99 |
| hs132692233 | TGTGTGTTAGGCCTGGACTGAGG | 100 |
| hs132684753 | ATGTTAAGCCCTCAGCATCGAGG | 101 |
| hs132686955 | GTAAGAGAAAAGTACCAACAGGG | 102 |
| hs132685684 | CATTTCTTGAGAAATCCTTGGGG | 103 |
| hs132691062 | AGAACAATAACACTACCACCCGG | 104 |
| hs132692511 | ACTGTGGTTTAAATGCAGTGAGG | 105 |
| hs132687555 | TTACCAGTACAGTACAGTGGTGG | 106 |
| hs132688050 | TAGAGCTGTGTGAATCCATGGGG | 107 |
| hs132691218 | CGATTAGGTCACAACCAATGGGG | 108 |
| hs132685069 | AGTGGACTTAGGAAGGTACAGGG | 109 |
| hs132692859 | GTGGAAATAATGGTAGATGTGGG | 110 |
| hs132693083 | TCCAAACAGACAGAAATAGGTGG | 111 |
| hs132687780 | ATATCTACTGTGAAATACTGTGG | 112 |
| hs132688699 | GGGTACCTGAGGGATCTGTGAGG | 113 |
| hs132684794 | GAGGTATTGAAGGAATCCAGCGG | 114 |
| hs132688163 | TCTTAAATGAGAGAAGCCCATGG | 115 |
| hs132689423 | AACAGTATTAGAGAAAACCAGGG | 116 |
| hs132689321 | GTGCCATAGAGCACTCTTGGGGG | 117 |
| hs132686643 | CTTGGCTGGATATCGAACAGTGG | 118 |
| hs132693602 | TTCATACACACTCATCATGGGGG | 119 |
| hs132692513 | TGTGGTTTAAATGCAGTGAGGGG | 120 |
| hs132686383 | GTATAACAAAGAAATAACCGTGG | 121 |
| hs132691243 | CAGGGTCAACAAGCTAACAGTGG | 122 |
| hs132691844 | ACCACAAACAGCACTCCACGTGG | 123 |
| hs132684425 | GACATCCAATGTGGTCTGTGGGG | 124 |
| hs132687714 | TGTGTGCAAAGGTAACAAAGAGG | 125 |
| hs132683904 | AAATGGCCCTATGCAGACAAGGG | 126 |
| hs132689023 | TTAGTAACAACCTAGTAACACGG | 127 |
| hs132691952 | GGATCAGAATTGAAACTGAGGGG | 128 |
| hs132692000 | GATGAATTCAACACACCCGGAGG | 129 |
| hs132692165 | ATGAATAGTGACCATGTGAGTGG | 130 |
| hs132693130 | GGTTGAGTGTTTATGCACAGAGG | 131 |
| hs132692978 | GCAAGAAAGATACAGTCTAGGGG | 132 |
| hs132693153 | ACTCTGTCTCTGTAATACTGAGG | 133 |
| hs132686520 | TAAGCCACAAGTGTAATGGGGGG | 134 |
| hs132687319 | AAAGCTTAAAGCTAACGGAGGGG | 135 |
| hs132694337 | GAATACGTAATTGAAAACCATGG | 136 |
| hs132693324 | GGGTAACAAAGTTAAAACCAAGG | 137 |
| hs132693986 | GTACATAAACTACCAATGGGAGG | 138 |
| hs132692198 | GTTGAAACCACATGTTGTGGAGG | 139 |
| hs132694406 | CTTCAGGTAGATGATGACAGGGG | 140 |
| hs132684128 | ACTACTTGAACACACCGCAGTGG | 141 |
| hs132684041 | TGGGACCCGCCTGTTCAGAGGGG | 142 |
| hs132693428 | AGACCCTACACTATGCAAGTGGG | 143 |
| hs132688850 | GATTGAGGGTAGAATGACAAGGG | 144 |
| hs132687303 | ACACTCACTCAATATCATAGGGG | 145 |
| hs132685186 | GAATGGCATAGAGCCCCCAACGG | 146 |
| hs132693008 | TGCTACCTATATATTCCCAGTGG | 147 |
| hs132689334 | AATGAGATAGGGATTCAACAAGG | 148 |
| hs132684482 | ATTTGGGTCAAAGAGAAGGGAGG | 149 |
| hs132688126 | TGGTCTTGTAGACTTACCCGAGG | 150 |
| hs132689750 | ATTGTGGCCAGCTCCCAGGGTGG | 151 |
| hs132688301 | TTTCAGCTAGAAATGAGCTGTGG | 152 |
| hs132690707 | ATGGTCATCCCATAGTACCAAGG | 153 |
| hs132694325 | TTATGAATGATATTCAACCACGG | 154 |
| hs132688352 | GCACTGCAGAGTTCCAGCAGAGG | 155 |
| hs132692070 | AGCTATAAGTGATTTAACAGGGG | 156 |
| hs132684543 | TATGGCAAACTAAAGAAAGGGGG | 157 |
| hs132684790 | GGACTTTGATGAGGACCATGCGG | 158 |
| hs132683980 | CACAAAGTTTGAACACATCGAGG | 159 |
| hs132688744 | CGGATGCTGGCCATAAACAGGGG | 160 |
| hs132687704 | AATTTGAAGAATAAGAACAGAGG | 161 |
| hs132692582 | TTGAGATACCAAAATAAACAGGG | 162 |
| hs132689530 | ACGAAAGCATGGTAGTTTGGGGG | 163 |
| hs132685501 | ATATAGTAAGAAACCCTGAGAGG | 164 |
| hs132692090 | TTATCCTAAAGCAATAGACAAGG | 165 |
| hs132685057 | TCAGCCGGGGAGCAAAACCAGGG | 166 |
| hs132691045 | AGAATGATCTGTAATCTCTGAGG | 167 |
| hs132689993 | AGAATTGCATAGTTATGACAAGG | 168 |
| hs132688078 | AAAACAGCATTGGTTCCAAGTGG | 169 |
| hs132691850 | AATCTAGGCAGCTATTCCAGGGG | 170 |
| hs132688469 | AAATAAGTAGTAAACAAAGGGGG | 171 |
| hs132687269 | ACCTTACAGTGCAAGCAGGGAGG | 172 |
| hs132689948 | AGATGATTACAAACAAACCAGGG | 173 |
| hs132690630 | TCACTACCAGGACCTTAGTGAGG | 174 |
| hs132690861 | ATAGCTATTAGGTCTATGTGAGG | 175 |
| hs132689879 | GTGGTGAGCAAGAACAGTTGAGG | 176 |
| hs132685189 | CCAACGGTACCAGCAATGTGGGG | 177 |
| hs132686308 | ATTTCAAACAGAAACCAGCAGGG | 178 |
| hs132684147 | TAAACACTGAGCCATGAGGTGGG | 179 |
| hs132686359 | TTGTCAATGGGTATGATACGTGG | 180 |
| hs132688977 | GGCTGGCTTGAAAATCAGGAGGG | 181 |
| hs132694162 | TCTCTATGGTAACAGACACAAGG | 182 |
| hs132691795 | TGACTGTTTAGGAGACACACAGG | 183 |
| hs132689131 | ATAACAGCAAGGGATAGGGAAGG | 184 |
| hs132685567 | CCATATTTTATAGAGAACAGTGG | 185 |
| hs132692790 | ATTCAATCACCTATACAATGTGG | 186 |
| hs132684558 | GGAAGATTGAGTGAATACACTGG | 187 |
| hs132686958 | ATGGGGTATGGTAAACCTGGAGG | 188 |
| hs132688973 | TTTCGCATAGACATCTGAGGAGG | 189 |
| hs132691044 | GGAGGCCTTATCAAACACCAGGG | 190 |
| hs132690439 | CCTGGAATTTGAAATATCCGGGG | 191 |
| hs132690818 | AAAACCTCAGTATGAAAACGGGG | 192 |
| hs132685239 | AAGTGATTGAAGGATGACAAAGG | 193 |
| hs132689450 | GGTAACCACAAAGTACCAGAGGG | 194 |
| hs132690736 | GAAGCCTGATGGAGATTAGGTGG | 195 |
| hs132683748 | TTGCTAATACTGATGACAAGTGG | 196 |
| hs132686015 | GATGTGGTATAAAAATTGTGAGG | 197 |
| hs132686527 | ACGAAAGGCATTATGGTAGGGGG | 198 |
| hs132693160 | TGATCCCTGAAATCCAGCAGTGG | 199 |
| hs132691006 | GGAGATGGCGCTAATCTGGGAGG | 200 |
| hs132689365 | TCTAGGGTGAAATTACACGCTGG | 201 |
| hs132694134 | ACAAGAACTAAATTTGCCAGGGG | 202 |
| hs132688730 | AAGGAAATAAAAGTATGGCGGGG | 203 |
| hs132689567 | TGATATCCAGCATAGATGGGGGG | 204 |
| hs132691976 | TCTACATTATGGAAACTCAGAGG | 205 |
| hs132686756 | TGAAGAGATAAAAACAAGCGGGG | 206 |
| hs132692645 | GAAAGCCATTCACCTCGAGGAGG | 207 |
| hs132692330 | AGGCCCTCAAATCTTCCTGGAGG | 208 |
| hs132689399 | GAAAACTTAGGAATTTACTGTGG | 209 |
| hs132685959 | ATTTTCCTAGCATATCTCTGAGG | 210 |
| hs132693504 | ATAATTGAGAATTAGTGCAGGGG | 211 |
| hs132694402 | CAGGGGGAAAAAAATCTTCGGGG | 212 |
| hs132687478 | GGAGAAAAAAGTACTCACAAGGG | 213 |
| hs132693775 | GCTTATATTAGCCAAAGCAGAGG | 214 |
| hs132686630 | AGCATGCTTCTTCACCACTGTGG | 215 |
| hs132685298 | ACACATAGATAGAACTCACACGG | 216 |
| hs132685330 | AAATTGAGATTAGAGCAACACGG | 217 |
| hs132689117 | GAACATGGCAGTTATAACACAGG | 218 |
| hs132687798 | ATATCAGGGCTATTGAGCAGGGG | 219 |
| hs132686189 | CCCCCAGTTAAAATGAACAGTGG | 220 |
| hs132689000 | TATTTATTGTTCCGACTCAGGGG | 221 |
| hs132690232 | AGTTACTCTCCCATTCGAAGGGG | 222 |
| hs132687956 | CATTGCTGTAACACAGACAAGGG | 223 |
| hs132689272 | TCTGGATCAGCATTCAACAAAGG | 224 |
| hs132691446 | AGTCCTCCCTGAGAATGTAGGGG | 225 |
| hs132687104 | TGTGACGACGATCATTGGTGGGG | 226 |
| hs132685245 | TACTCGATCCTACAAGTGCGTGG | 227 |
| hs132687374 | AGATGATGAAGTTTGGACAATGG | 228 |
| hs132693650 | GCCACAGGGACAGAAGTAAGCGG | 229 |
| hs132687494 | TAATAGCAAAGAGGTAAACGTGG | 230 |
| hs132684350 | TAAACAACCCTAAGGCAGTGAGG | 231 |
| hs132690753 | GTAAAAAAATAATTCTAACAGGG | 232 |
| hs132690298 | CAACACATCTCTTCATACAGAGG | 233 |
| hs132694401 | AATTGCATATAACTAATCTGGGG | 234 |
| hs132691551 | ATATAGTTCTTATAATAGTGGGG | 235 |
| hs132686150 | ATACCAAGTATGGTAACACATGG | 236 |
| hs132687025 | AATGAAATAAGTATAGAACATGG | 237 |
| hs132685246 | ACGCACTTGTAGGATCGAGTAGG | 238 |
| hs132685880 | AGCAAGGAGTAGATACAGGGAGG | 239 |
| hs132690686 | AGGGAGCATATAATACACAAAGG | 240 |
| hs132690438 | CTGGAATTTGAAATATCCGGGGG | 241 |
| hs132684212 | GCAGCAGTAACAAACGAACAGGG | 242 |
| hs132683807 | AAATAGTGCCCAATCCTAGGTGG | 243 |
| hs132689451 | TGGTAACCACAAAGTACCAGAGG | 244 |
| hs132690159 | GTTCCCATTAGTGAGGAATGGGG | 245 |
| hs132684260 | TTGCAGATTTAGATTAACTGGGG | 246 |
| hs132685059 | TATGTTCTGTACATCAGCCGGGG | 247 |
| hs132690276 | AGTCCTGCTATAGTCCTAGGAGG | 248 |
| hs132694068 | ATGACAACTGGAAACGTAGGTGG | 249 |
| hs132690916 | AAAATAGTATGAGCATGCAACGG | 250 |
| hs132687720 | AAAAACAAAACATATCACAAAGG | 251 |
| hs132688858 | TGACAATCATGGGAACGACAAGG | 252 |
| hs132688159 | TGCCAACCTGAAGAACGTGAGGG | 253 |
| hs132684399 | GATAAGCAGCTAACATGTGTCGG | 254 |
| hs132692272 | ATAATGTGAAGTAATATGCATGG | 255 |
| hs132686899 | TGTCTAAATTACTATTCACAAGG | 256 |
| hs132690382 | AAGGATTTAAAAATGGACTGTGG | 257 |
| hs132690474 | ACTTTTGATTGAATCCCAAGGGG | 258 |
| hs132692457 | CAGCCTCTGAGCCCTCAAGGTGG | 259 |
| hs132689320 | TGTGCCATAGAGCACTCTTGGGG | 260 |
| hs132687089 | GTGTCCTTGAAGGTGCATTGTGG | 261 |
| hs132684229 | GGTTCAGTGAAACCTTCGTGAGG | 262 |
| hs132689969 | AATAGTGCTATGCACACACAAGG | 263 |
| hs132689815 | CAATAAGTAAGCTTGTGAAGGGG | 264 |
| hs132686337 | AAAACAACGACGAAGTGGGGAGG | 265 |
| hs132688196 | CCTATTCTGAGCATGCCCAGTGG | 266 |
| hs132687490 | GTAAACGTGGCAGAAAGCAGGGG | 267 |
| hs132684310 | GCATATAAAGCAGACCCAAGAGG | 268 |
| hs132693134 | ATATAAAAGAGAATTTAGTGAGG | 269 |
| hs132692661 | AGGCCATTGAACAATTTGAGGGG | 270 |
| hs132686776 | GATATGATTCCTTCACCATGAGG | 271 |
| hs132685390 | GTATAATATCAAAAAGATGGGGG | 272 |
| hs132687439 | TATGGGTAAATATAAGAACATGG | 273 |
| hs132690966 | AACTCTCTGATGGAGTTGTGGGG | 274 |
| hs132691824 | GCGAAGCCACTACCCATGGGGGG | 275 |
| hs132688464 | ATATGTACTGAAAGCAACTGCGG | 276 |
| hs132690082 | AAGAAATGTTGGCTTGTAGGTGG | 277 |
| hs132686839 | ACAACGCATAAAGAGAACACAGG | 278 |
| hs132691575 | AATCTAAGTGGAAGTCCAAGGGG | 279 |
| hs132688947 | CCTTGTAGTTATCCCACATGGGG | 280 |
| hs132690881 | GGGTGACGTGCATATGTGCGTGG | 281 |
| hs132687149 | AGACATTTAATATATGCCCAGGG | 282 |
| hs132693609 | TAGGAGTTCAGAGCATGGTGAGG | 283 |
| hs132689128 | GATGTGGTGGATAACAGCAAGGG | 284 |
| hs132685420 | AAGATGGCGAATATTGACAAGGG | 285 |
| hs132691757 | AGATCCGAACAAACCATCAGCGG | 286 |
| hs132690960 | TAAGGGTGAGTAGTAACAGGAGG | 287 |
| hs132691924 | TCTAATCTCTGTAAGCCAAGTGG | 288 |
| hs132684712 | GCATCAATAGCTAATTCTAGGGG | 289 |
| hs132694065 | TACACCTGAAGGTAAATGGATGG | 290 |
| hs132692819 | TCTTCAGCTATGACCAGACGTGG | 291 |
| hs132691763 | ATAACAGCCAGAGTAATACCAGG | 292 |
| hs132684364 | AGTGAGAGCTAGAAGCCGTGTGG | 293 |
| hs132691725 | CATTGTGTAAAACAATATAGCGG | 294 |
| hs132692184 | TTGGTTGGTATTGATCAGAGAGG | 295 |
| hs132691556 | ACAAGATTGAGGAACAAAGGTGG | 296 |
| hs132686549 | TTAGAACCAAAAGTCGATGGAGG | 297 |
| hs132692419 | ATGTAATCTACGAATCATTGGGG | 298 |
| hs132693290 | CAATTTGTCAAAGCTGACTGGGG | 299 |
| hs132683441 | AGGGGTTAAAGATTACTGAGAGG | 300 |
| hs132687739 | TCTATGATGAAGGAAATGGTGGG | 301 |
| hs132684551 | TAGGATGTGATATCTGGCAGTGG | 302 |
| hs132689071 | AGAAACTTAACAGAAAAAGGAGG | 303 |
| hs132690425 | CCAGATCTGAGTCCTGTAGGGGG | 304 |
| hs132692748 | CTTAGGAGTAATTACCACGGTGG | 305 |
| hs132693208 | CCAAAGTTGGGCAAAGACTGGGG | 306 |
| hs132683725 | ACTTCCGTATTTGGTGAACAGGG | 307 |
| hs132692569 | TACAGGCCATGGGAATGATGGGG | 308 |
| hs132688774 | TGGGAGAGATTACACTCCCGTGG | 309 |
| hs132686587 | ATTAGTCATGGAGTCCTAAGGGG | 310 |
| hs132689161 | AGCTAAGCCACTGATTACTGTGG | 311 |
| hs132693190 | GATAATAGCTGAACATTACGTGG | 312 |
| hs132688165 | GCTTCATCAGAAACCTAGTGTGG | 313 |
| hs132690726 | TGAAGACTTTCGTCTCAGAGAGG | 314 |
| hs132687321 | GAAAAGCTTAAAGCTAACGGAGG | 315 |
| hs132686543 | AGCCGTTAGAGAAAGTGTTGTGG | 316 |
| hs132686677 | GAGGGTGAGGAAACTCACACGGG | 317 |
| hs132687746 | GGTAAGAGTTAGTTACACATGGG | 318 |
| hs132685845 | AATTACGGTGCTACAAGACGAGG | 319 |
| hs132684266 | GAGCTTCCACTAGGCCAGAGGGG | 320 |
| hs132684724 | CCTAGAATTGACGTTACAGGAGG | 321 |
| hs132692162 | ATGGAATGCAGTGTTCAGAGTGG | 322 |
| hs132690034 | GGAACAACAGGAAGGATTAGGGG | 323 |
| hs132694069 | AGGATGACAACTGGAAACGTAGG | 324 |
| hs132690361 | TTTCCACAAAGTTTTCCCAGTGG | 325 |
| hs132690142 | TGGTTGTGTCAACGAAGAGGAGG | 326 |
| hs132693649 | ATGTTGGAGCAATAGCCACAGGG | 327 |
| hs132693054 | GCTAGAAGTAGAAAAGCAGGAGG | 328 |
| hs132685703 | AAACGAATATTGGCGCCATGAGG | 329 |
| hs132684324 | TGATATGCTAAATAAAAGGGGGG | 330 |
| hs132692298 | AAATAAGGTGGGATCCAAGGTGG | 331 |
| hs132685844 | TACGGTGCTACAAGACGAGGAGG | 332 |
| hs132690766 | ATTGCGGTGCCTAAAGCTCGAGG | 333 |
| hs132685660 | AAGTTTCCTCCCTAGAGGAGGGG | 334 |
| hs132687767 | CATCCAAATACCCCTCATAGGGG | 335 |
| hs132692237 | GTCCAGAACATGTTACAACAGGG | 336 |
| hs132690048 | CCATGAAATGCACACCTGGGTGG | 337 |
| hs132693576 | GCCAATTATCTAATTCTCAGGGG | 338 |
| hs132690254 | CTATTAGTTAGACTTCCTTGGGG | 339 |
| hs132692646 | CTGGAAAGCCATTCACCTCGAGG | 340 |
| hs132690892 | ATATGCAAATCAGTATGGAGTGG | 341 |
| hs132691225 | ATACAGTACTAAACAGCCTGAGG | 342 |
| hs132690776 | GAGTAAGTATATTCATTGTGGGG | 343 |
| hs132693450 | GCTCAATCTACTAAGTAACCAGG | 344 |
| hs132687944 | AGCTTGTTGAGAGCCACACACGG | 345 |
| hs132690132 | ACATGGGCATATCAACAATGAGG | 346 |
| hs132689141 | TCAAAAGCAGGATCTTACAAAGG | 347 |
| hs132685645 | CCTCAATTAGAAAAATGCAGAGG | 348 |
| hs132690065 | GGTGTGTACACCTAATCCAAAGG | 349 |
| hs132684631 | ATAAACATGTGGTAGTGGTGAGG | 350 |
| hs132691280 | AATGCTTGTAATGGATGTGACGG | 351 |
| hs132687333 | CAAAACCCATCAACTATAGGCGG | 352 |
| hs132687477 | GAAAAAAGTACTCACAAGGGAGG | 353 |
| hs132692353 | GTTTATGCTCTCCCGCACAATGG | 354 |
| hs132694289 | TGGATTGGACAGACGCTAGGTGG | 355 |
| hs132692756 | AGATTGTTACGGAAGTTTTGGGG | 356 |
| hs132692863 | GTATGACAAGAGACATCCATGGG | 357 |
| hs132690943 | GTGACAAGCTTAGCGAACAGTGG | 358 |
| hs132694217 | TAGTACTTAAGTAGTATGGGTGG | 359 |
| hs132686953 | TTTCTCTTACACATTCTATGGGG | 360 |
| hs132693559 | CCTTTACTGAAATCAGACAAGGG | 361 |
| hs132684360 | CCTACCAAGAGAAGAAGAGGGGG | 362 |
| hs132689568 | GTGATATCCAGCATAGATGGGGG | 363 |
| hs132688082 | TAAATAAATATACTAACCAGTGG | 364 |
| hs132690533 | TGGTGCTTTAGACATTACCCAGG | 365 |
| hs132685901 | TCAAGCAACACTCACACGGGTGG | 366 |
| hs132688101 | AGTGCATACTCTAGTAGTGACGG | 367 |
| hs132691956 | ACGAAGGAGAAAATTCTTTGGGG | 368 |
| hs132684538 | AGAACTGGTAAACAACTGCAGGG | 369 |
| hs132687785 | ACAAACCAGGAAAAATGAGAGGG | 370 |
| hs132694405 | TTCAGGTAGATGATGACAGGGGG | 371 |
| hs132690112 | AGTCTTAGCCAAAAGCAGAGTGG | 372 |
| hs132686174 | TCACTTTTAGTGTTCCCCTGAGG | 373 |
| hs132689675 | TGATTACATTAGCTTCAGAGTGG | 374 |
| hs132686553 | GGAAAAAGATTCCAGAAAGGGGG | 375 |
| hs132690077 | ACGGGAAGCTCAGACAGCTGGGG | 376 |
| hs132692946 | GTGCCTATCCTAATAAGCAGGGG | 377 |
| hs132687052 | TACTAAGTGAATGGAAATGGAGG | 378 |
| hs132684124 | TCCATTATTTTTGACCACTGCGG | 379 |
| hs132690009 | GCAATATGGGAGGAAATTAGGGG | 380 |
| hs132693092 | AATTTTAAAAAGTTTCACAAGGG | 381 |
| hs132688933 | AATCCTTCTGATACAGAAGGTGG | 382 |
| hs132683301 | GAATAAATACTGTTATTCAGAGG | 383 |
| hs132693475 | GTAGATATATACATAAATGGGGG | 384 |
| hs132692372 | TAATTATGTGTCAACACCTGAGG | 385 |
| hs132694152 | CACTGAAGACTATAAAACTGAGG | 386 |
| hs132693536 | CATAATTCTCAAAAAGAGGGGGG | 387 |
| hs132688605 | CATTGATGATGCCTTGAGTGTGG | 388 |
| hs132694275 | TGGGATTTGAAGACTTCACGTGG | 389 |
| hs132687902 | ACAAGGGCAGTGATAAGGAGAGG | 390 |
| hs132694302 | ACTATGGTAACAAGAGAGTGAGG | 391 |
| hs132692001 | ATGAATTCAACACACCCGGAGGG | 392 |
| hs132684000 | ATTCTCAAGTGCAGTCACCAGGG | 393 |
| hs132691315 | ATTTATGGCATAATTACCAGAGG | 394 |
| hs132683560 | GCAGTATGATGAAGTGCAGGGGG | 395 |
| hs132685408 | TGTCTATTAAGCATCAACCTAGG | 396 |
| hs132691318 | TATAGCTGTATCATTTACTGAGG | 397 |
| hs132688107 | GTTACATTTAACCTTCACCATGG | 398 |
| hs132686513 | AGTTTTCTACCATAGACAAGGGG | 399 |
| hs132690064 | CTGACAGCCCATTAGAGACGGGG | 400 |
| hs132690041 | AATGGCACCATTATCCACCCAGG | 401 |
| hs132692217 | AATTCTTTCCCAGAACAATGTGG | 402 |
| hs132683561 | AGCAGTATGATGAAGTGCAGGGG | 403 |
| hs132686456 | TAAACTGCAGACACTTCCACGGG | 404 |
| hs132684593 | TAAAAAATGAGAGATAGGTGGGG | 405 |
| hs132692125 | ACACTTGAGAAGAAACTAGACGG | 406 |
| hs132693874 | GATGGAGTCAACATTTCCAAAGG | 407 |
| hs132692624 | ATAAATATCCTTGATGCCTGTGG | 408 |
| hs132688604 | GTGGAAAACAGAAGTCAATGAGG | 409 |
| hs132690481 | TGTGTGTGTATAGGTCTCATGGG | 410 |
| hs132685498 | ACTGAACAATTGGTATACTGAGG | 411 |
| hs132692326 | ATACAGATATAGCACATACCAGG | 412 |
| hs132691826 | ATGCGAAGCCACTACCCATGGGG | 413 |
| hs132692109 | TCATTTGCATGGCTCTACGTGGG | 414 |
| hs132688700 | GTATAAGACAGGGTACCTGAGGG | 415 |
| hs132689699 | GGGCTCACATTGTAGAAATGTGG | 416 |
| hs132690143 | ATGTGGTTGTGTCAACGAAGAGG | 417 |
| hs132685663 | CTGACAAGTTTCCTCCCTAGAGG | 418 |
| hs132686622 | GTGAAAAGTGATGTGAGACAAGG | 419 |
| hs132685752 | GTAATTTTGGTATATATACGTGG | 420 |
| hs132693717 | GTGCTCGGCATGAATGCAGTGGG | 421 |
| hs132683869 | GTCTGCTACATGAGGAACAGCGG | 422 |
| hs132687706 | AAATCAACACCGATAATACAGGG | 423 |
| hs132685225 | TCCTGCCTGATACAACCAGAAGG | 424 |
| hs132691463 | TGACTCATCACATCCTGGAGAGG | 425 |
| hs132683653 | CAGACAAATGGTCAAACACAGGG | 426 |
| hs132689941 | TGAACAAAAAGCTAAGAACTGGG | 427 |
| hs132687781 | TATTTCACAGTAGATATCCAGGG | 428 |
| hs132687783 | TGTGGTCTCACATACAAACCAGG | 429 |
| hs132693826 | AGGTCATGTGTAAGTCACAAAGG | 430 |
| hs132688758 | AAGCAACTAGCAATAGACAAAGG | 431 |
| hs132684215 | AAAGGCTGCCAGAGTAACACGGG | 432 |
| hs132689549 | ACATGACCTTACATACATCACGG | 433 |
| hs132692278 | ATTGCCTCTCATATTTACAGAGG | 434 |
| hs132683844 | TTGGCATTGTATAAACATCAGGG | 435 |
| hs132687584 | GGCCTTTGAAGAAATCACGCTGG | 436 |
| hs132692319 | GTGTTTCAAACACTACTGATGGG | 437 |
| hs132686754 | CTTGAAGAGATAAAAACAAGCGG | 438 |
| hs132694328 | CCTTGAAGCATACATTACCGTGG | 439 |
| hs132688081 | TCTTAATTAATGTTACTGAGAGG | 440 |
| hs132688978 | GGGCTGGCTTGAAAATCAGGAGG | 441 |
| hs132690988 | TCACAAAAAACGTTGTGTAGGGG | 442 |
| hs132690877 | AAAATACTCATGAAGTACCAAGG | 443 |
| hs132693471 | ATATGTGAGTAGAATACACTGGG | 444 |
| hs132691959 | ATCCTCCAGATTCATAACGAAGG | 445 |
| hs132690513 | ATTTAGACAACCACACTGAGAGG | 446 |
| hs132683557 | ATAATTCTCCCATCTAAGTGTGG | 447 |
| hs132688044 | ATTAGATGAAGAAAATCACATGG | 448 |
| hs132687378 | ACACGTGTTGGACGTTAGTGGGG | 449 |
| hs132690727 | AGGTACCTAATAGCAAGACCAGG | 450 |
| hs132692934 | AGTAAGAATTGAGATATGTGCGG | 451 |
| hs132694240 | GACCAACTAAAATATTGTCGTGG | 452 |
| hs132689036 | AATACATTAAAACCAGACAAAGG | 453 |
| hs132689339 | ATCTGGCCTAGGCTACATGTAGG | 454 |
| hs132688542 | AAATCAGTGATACATATCGATGG | 455 |
| hs132694252 | TCTGAAGAGCAACACCTGAGAGG | 456 |
| hs132690388 | GTAAACGACATACAAACTTGAGG | 457 |
| hs132690870 | TTAAAATCTAGAGTATTACAGGG | 458 |
| hs132686114 | GAAAGAATTTGAATACCAAGAGG | 459 |
| hs132694145 | ATTGAGAAAAAGTTGTCGTGGGG | 460 |
| hs132686478 | GTTATCAAAGTGTTAAACACAGG | 461 |
| hs132685730 | GTTGTATGATTGGATGTACTGGG | 462 |
| hs132687835 | ATGGCTGCATGATAATTAGTGGG | 463 |
| hs132693387 | ATACACGATGATCTAGTACAAGG | 464 |
| hs132687046 | GCAGTACTCTTTGTAGACAAGGG | 465 |
| hs132692877 | TGTGGGAGACATCACCACAATGG | 466 |
| hs132685312 | CATAACTAGAGAGGAAATGGAGG | 467 |
| hs132694257 | GCAGCTACTCTAGCTCATGTGGG | 468 |
| hs132684154 | CTAAAAAGTACAAGACTGTGAGG | 469 |
| hs132693256 | TGTAGCTTAGTCTCACAGCAAGG | 470 |
| hs132684554 | AGTGGTGATTCCAATCATTGGGG | 471 |
| hs132690013 | AGACACCTAATGCTAAAAGTGGG | 472 |
| hs132691063 | TGCACAACAAAAGCTTAACATGG | 473 |
| hs132691059 | GCTAGTGGGGAAGGACCGGGTGG | 474 |
| hs132689854 | ATCATCACCAAACCTAATCGAGG | 475 |
| hs132683413 | TCCTATGAATGACATACCTGCGG | 476 |
| hs132688454 | ATTTCACCTAAATTACACAAAGG | 477 |
| hs132689569 | TGTGATATCCAGCATAGATGGGG | 478 |
| hs132693589 | CATCCATAATCAGAGAAGGGAGG | 479 |
| hs132689464 | GATCCAAAGACCATTTCCCAGGG | 480 |
| hs132687951 | CCACATGTAAAAATCTCCAGAGG | 481 |
| hs132690167 | ATAATGACAGAGTGAAAGTGAGG | 482 |
| hs132694379 | GGGTTCTAAAAATCGTACGCAGG | 483 |
| hs132688825 | TCATATAATACCATAAGCTGAGG | 484 |
| hs132692979 | AGAAAGATACAGTCTAGGGGTGG | 485 |
| hs132693575 | GAAAATACTCAGTAAGACTGAGG | 486 |
| hs132687744 | TAGATTGAAAGCATTAACAATGG | 487 |
| hs132691943 | CAACCAGCAAAATAACCCTAGGG | 488 |
| hs132687498 | TTTGAAGGTTGACAACTAAGGGG | 489 |
| hs132692982 | GGGTGGACAGGTAAAGAACAGGG | 490 |
| hs132683465 | TGAAAGCATTTAAAAACTGGGGG | 491 |
| hs132683683 | CTGAAAAGAGGAAATAATCGTGG | 492 |
| hs132687953 | AGTAATTCTCTGTAGGCAAGCGG | 493 |
| hs132688972 | GCCTTTCGCATAGACATCTGAGG | 494 |
| hs132683610 | TGTTTGCACAATCTACCAGATGG | 495 |
| hs132690379 | ACTGTGTGTCCCGAAGAGATGGG | 496 |
| hs132692067 | GATTTATAATGATTACCAAGTGG | 497 |
| hs132690093 | TTGCCTTTGATGAAAATACGTGG | 498 |
| hs132690511 | AACCACACTGAGAGGAAAGGAGG | 499 |
| hs132683857 | GGTTCCAAAAAAAAGCAGAACGG | 500 |
| hs132694375 | TTATAATCCACTGATTGTAGGGG | 501 |
| hs132692966 | GGAGCTGCAGCTTTTCAAAGGGG | 502 |
| hs132691903 | TCTGCTGGATGAAAGAACACAGG | 503 |
| hs132688015 | GATTAAAAATCACCAAAACAAGG | 504 |
| hs132691624 | AACCATGCTTGACGCTAGGATGG | 505 |
| hs132691545 | AGTCTATATCAAAATATAGGGGG | 506 |
| hs132690529 | TATGTTTCTAAAAACAACCGTGG | 507 |
| hs132693056 | TTAAAATAACCATTATCACCAGG | 508 |
| hs132693121 | AATGTGACTTATGAGACTGAAGG | 509 |
| hs132691156 | ATTCACAATTGACCATTTCGGGG | 510 |
| hs132684629 | GTAGAAATGGAATAAACATGTGG | 511 |
| hs132689284 | TGTTTTGATAGCAATACCTGAGG | 512 |
| hs132687376 | TTAGTGTACTGAGAAATGAAAGG | 513 |
| hs132690518 | CAGTGTCCCTCTATGCTGTGAGG | 514 |
| hs132685707 | CTTTACAAAATGATAAACCCAGG | 515 |
| hs132693840 | TTTCCCTAAACTGAATGAGGAGG | 516 |
| hs132684656 | AAGTAAATCAGTCCATCACAAGG | 517 |
| hs132690453 | ATAAGGTTGTGCTTCCAGCATGG | 518 |
| hs132686615 | GATAGCTAGGCTAATTCCAAGGG | 519 |
| hs132685314 | GCACATTCACATAACTAGAGAGG | 520 |
| hs132687287 | GAGAAGCCAAAATCATAGCACGG | 521 |
| hs132688112 | ATGGCAGGAAGATTTATTGGGGG | 522 |
| hs132687130 | CTCCAAGCTAAAAATTCATGGGG | 523 |
| hs132688360 | TTCTAAACACGTATTCTGTGAGG | 524 |
| hs132693754 | GTTGGGTTAAGCAGTGACATGGG | 525 |
| hs132690566 | GCCATCTATTTAATAACACAAGG | 526 |
| hs132683517 | AGCCTGAAAATATAGCCAGAGGG | 527 |
| hs132686675 | AGTACCTAAAAGTGAACAGGAGG | 528 |
| hs132693990 | TGACCATGCAAACTTTCAGGAGG | 529 |
| hs132683438 | GAGAGAATAAAATAAAACGTTGG | 530 |
| hs132689647 | TTTTCAAATTTAAGCCAGAGGGG | 531 |
| hs132686213 | GGGAAGCTTATTGAAGACAGTGG | 532 |
| hs132694354 | CCAGTCTCCTCCATAGCCTGAGG | 533 |
| hs132685335 | AAAAAAATACAATGGTAAGGGGG | 534 |
| hs132693262 | ATTAGGCTAATACTGGTCTGTGG | 535 |
| hs132690162 | TTCCTCACTAATGGGAACTGGGG | 536 |
| hs132690059 | TGAACCTCTGTGATAACACTTGG | 537 |
| hs132686210 | TTATGAGGCAAATAATGAGGAGG | 538 |
| hs132692879 | CAGACAGTGATGTTTCATGTGGG | 539 |
| hs132693069 | GGTATTACCTGTAAAAACCAGGG | 540 |
| hs132688105 | GCATATGGTATGTGCTAGCGTGG | 541 |
| hs132694362 | TATCTTCACATGCTGTCATGGGG | 542 |
| hs132685043 | AAAATGTTAAGTACTGAGGGTGG | 543 |
| hs132688817 | AGTTTAAAGTGTCTTCTCAGTGG | 544 |
| hs132688684 | AATGCTACAGTAGTACAGCTGGG | 545 |
| hs132684366 | TGAGAGCTAGAAGCCGTGTGGGG | 546 |
| hs132687544 | ATAGTACAAAGATGTTCAGGAGG | 547 |
| hs132694195 | TAAACCCACGAAGAGAGAAGAGG | 548 |
| hs132688437 | AACCCAAACTGACATGTAGGAGG | 549 |
| hs132687658 | CACGCACAAGAGATGTGGAAAGG | 550 |
| hs132692655 | TCACAATGGGATGTACACTTGGG | 551 |
| hs132692654 | GCAGTGAGAAGAATATAGAGAGG | 552 |
| hs132689877 | TTTGGTTCCAGGTAATAAAGTGG | 553 |
| hs132693541 | ACTAACAGACTAATGGAAGGAGG | 554 |
| hs132687659 | TTTAGCACGCACAAGAGATGTGG | 555 |
| hs132691818 | CTACTGTGTCTGATATGTGGAGG | 556 |
| hs132684784 | TGAAAATAGAAAGTCCCGCATGG | 557 |
| hs132692008 | CCATTATTACAACACTCTAGAGG | 558 |
| hs132688987 | ATACACAAGAATCTTGGCAAGGG | 559 |
| hs132685559 | ATAACAATAATTAATACTGGTGG | 560 |
| hs132688671 | TCTGTTTTTTCCCCAAATTGCGG | 561 |
| hs132692115 | GGTTGATTGTAAATGTCCCAAGG | 562 |
| hs132683505 | GTGGTCAGCTTACAATGAGGAGG | 563 |
| hs132688690 | ATACAATAGACTGCAACCAAAGG | 564 |
| hs132684687 | GTAAAACTAACATTTACCTGTGG | 565 |
| hs132693573 | TAATTGTTTCAGTAGAACAGTGG | 566 |
| hs132687108 | GCTTGGCCTCATTAGCTCTGGGG | 567 |
| hs132688413 | ATCGTGTGTCTATATTTCAGAGG | 568 |
| hs132683617 | AATATATTACAACTTCCCGTGGG | 569 |
| hs132689807 | ACGCACAAACATAAAATCAGAGG | 570 |
| hs132686850 | AATGCATGTACAAACTAAGGAGG | 571 |
| hs132687906 | TATTTGAACTCCATGCCAGAGGG | 572 |
| hs132686336 | CTGAAAACAACGACGAAGTGGGG | 573 |
| hs132693102 | TTATATGTAAGTCTCTTCGGTGG | 574 |
| hs132688092 | CTGTGATTTTGTGAACACAATGG | 575 |
| hs132688936 | GATTGTTCTGATAACCCAAGAGG | 576 |
| hs132692246 | GTGGCACCCATCTTAATGAGAGG | 577 |
| hs132692037 | TAAGTTCAGGAGTTTTAACGTGG | 578 |
| hs132693376 | CAGATATATTCCTAGGAGGGTGG | 579 |
| hs132694299 | ACACAGTTTGGGGATTCCAGTGG | 580 |
| hs132691202 | CCAGCCAGTTCTCTAGACTGAGG | 581 |
| hs132692452 | TGTCTATGAAAAGGAGTAGGAGG | 582 |
| hs132686205 | CCAAAACATAAACATTCACAGGG | 583 |
| hs132692069 | TAGCTATAAGTGATTTAACAGGG | 584 |
| hs132689045 | ATAAGGTATTTATTACCACTGGG | 585 |
| hs132685276 | GTTTCTCAAAGAAAGACCCCAGG | 586 |
| hs132690222 | TATGAAATACTTTAACACCCAGG | 587 |
| hs132684644 | AGAGATTGATTGGTTGACAGTGG | 588 |
| hs132692053 | ACCTACATAAAAATAAGGGGAGG | 589 |
| hs132688396 | ATTACACACAAAATAGTGGACGG | 590 |
| hs132689496 | ATGCTCATCTCTAGGTATGGGGG | 591 |
| hs132693338 | CCATTACCACAATAGTCCAGTGG | 592 |
| hs132693550 | TGTAAAGGATTAGGTAACTGAGG | 593 |
| hs132689144 | GAGAAAGTGTGGAAAGCACTGGG | 594 |
| hs132690446 | ACCAACTTGAGAGAGAAGGCGGG | 595 |
| hs132693824 | TTAAATTAAGACCTCTGGGGAGG | 596 |
| hs132684280 | ACAGTTAACTAGAACTGCTGGGG | 597 |
| hs132692110 | AGAGCCATGCAAATGAGAGTGGG | 598 |
| hs132686460 | TAACTCTTAACCATTTTGAGGGG | 599 |
| hs132683989 | AGGGAATATGAAACCCCCATAGG | 600 |
| hs132691050 | GCAAAAACTCCCAAATGGTGAGG | 601 |
| hs132692199 | ACATGTTGTGGAGGAACTGATGG | 602 |
| hs132685384 | AATTACATCAACAAAAATGGAGG | 603 |
| hs132694270 | TAAAGCACTTCAGAAGACACAGG | 604 |
| hs132686017 | CTTAGCATAACCATAACCTAGGG | 605 |
| hs132689989 | GAAATGTCTAAAAATTCTGGAGG | 606 |
| hs132687190 | GGGCATTGCACCTTGCACAAAGG | 607 |
| hs132692216 | GAAAGAATTGAGACATTACTGGG | 608 |
| hs132692613 | GGGTATGGAGTGGATCAACATGG | 609 |
| hs132683363 | CATCTTCATAGACTTTATGGAGG | 610 |
| hs132694392 | TCAAAAGCTAGAAATTGATGAGG | 611 |
| hs132686432 | TCCTATATACTAGAGCCAGAAGG | 612 |
| hs132688483 | TCTAGTGGAGCAGTAACTGAGGG | 613 |
| hs132689914 | ATATATTTCGGAGACCCAGTAGG | 614 |
| hs132685586 | TATCCCTGAGAGTATATGAGTGG | 615 |
| hs132687241 | GCTACAATAATTGTTCTGAGAGG | 616 |
| hs132687184 | CCTTTAAATGAGCACAACTGGGG | 617 |
| hs132690260 | CATTGAGCACACTCTACAAGGGG | 618 |
| hs132686351 | GCATAACTCTCTCAGTCCAAAGG | 619 |
| hs132691241 | TCTAGTCCTCAGCATCATCAGGG | 620 |
| hs132686751 | AAGATTAAACATGAACCTGTGGG | 621 |
| hs132687550 | AATTATAGGGATAAACACCCAGG | 622 |
| hs132684436 | TTATTCCAATCCTAGAGTGGAGG | 623 |
| hs132693755 | ACGTAATGATGTCAACCAGTAGG | 624 |
| hs132693997 | CAGTGAAGATGAGCAACAGGTGG | 625 |
| hs132691394 | TTTCTCAGTTGGTATCTCTGGGG | 626 |
| hs132690280 | TACTGTATAAAAATAAACCAAGG | 627 |
| hs132684915 | TTTTCATAAATGAATTACTGGGG | 628 |
| hs132686106 | TTTTTTAAATTATATTCACAAGG | 629 |
| hs132686518 | TGTAAGCCACAAGTGTAATGGGG | 630 |
| hs132691626 | ATATTTTTCCTAGAAAAGCGTGG | 631 |
| hs132685910 | GTCTGATAAAACCATAACAAGGG | 632 |
| hs132693396 | ACCCTACGCATAAGAGCAGGTGG | 633 |
| hs132692949 | GCTACATACTGTAATCACTCAGG | 634 |
| hs132691100 | GATAATACCTACATAATACTGGG | 635 |
| hs132684367 | GAGAGCTAGAAGCCGTGTGGGGG | 636 |
| hs132688632 | AAAGCATGTATGTAATTGGGAGG | 637 |
| hs132688151 | ATAGGCAATAATATTATGTGAGG | 638 |
| hs132686408 | TATTATTTTATAGTTCAAAGGGG | 639 |
| hs132690757 | CTTGCCATGGAAAAAAATGGTGG | 640 |
| hs132684654 | TCTGTGCCCTTGAAGGCTAGAGG | 641 |
| hs132693143 | CTAGTTGATTATGATCCCCAAGG | 642 |
| hs132693643 | CGACAGACACCAAATGCCAATGG | 643 |
| hs132688776 | GATCTTACTTGATACTACCACGG | 644 |
| hs132683757 | CTATTGGCAATGTACACTGAGGG | 645 |
| hs132684038 | TACTAAACTCTACCACCCATGGG | 646 |
| hs132691096 | GCTAGAACCCAGCTAAGAGAGGG | 647 |
| hs132690424 | CCCAGATCTGAGTCCTGTAGGGG | 648 |
| hs132692395 | CATGTCATATGCTTTCCCATGGG | 649 |
| hs132690666 | AGGCAAAGGCTACATTGCGTAGG | 650 |
| hs132693040 | GATTATAGAAGTTTACATTGGGG | 651 |
| hs132690434 | CTGATGCCAGGATTCTGTAGAGG | 652 |
| hs132690890 | ACTGTTCGCTCAAGAAGTAAGGG | 653 |
| hs132684460 | AATGCTATGTGAGATAGGAAAGG | 654 |
| hs132692888 | AAAATGTGAATAGTTCTGAGAGG | 655 |
| hs132688783 | AATAAAATAGAGAAAAGACTAGG | 656 |
| hs132686683 | GCACTTAAGATGTTAGAGGGAGG | 657 |
| hs132684858 | CGAGACATAGTCTCTGCCAATGG | 658 |
| hs132685900 | AAATCAAGCAACACTCACACGGG | 659 |
| hs132687670 | TGTTAAACATCAATAGAGGGAGG | 660 |
| hs132684887 | CAATACTATTAACAACCCCAGGG | 661 |
| hs132694251 | TGAGCAGGGGAAGACACTATCGG | 662 |
| hs132687344 | GAAATTCTCAGATAATCTCAGGG | 663 |
| hs132690098 | TTAGCCTTGGAAGCTGTGTGAGG | 664 |
| hs132691640 | TCCGTTACACGTTTACAAAGGGG | 665 |
| hs132692360 | CTTGCCCCAATGCCATTGTGCGG | 666 |
| hs132683495 | AAAATGGCAGTGCAGAGTGGAGG | 667 |
| hs132683965 | TCTACCAATGCCGGGCTCCGGGG | 668 |
| hs132690353 | AATTCTGCTGAAATTAAGCAAGG | 669 |
| hs132684148 | AGTGTTTAGTCATATAGCAGAGG | 670 |
| hs132691510 | GACTAGATGACCATCAGAGACGG | 671 |
| hs132684901 | GAATAGAAAAAGTCTTACAAAGG | 672 |
| hs132687907 | CTATTTGAACTCCATGCCAGAGG | 673 |
| hs132683641 | GTATTTTTTTAAATTTTCAGGGG | 674 |
| hs132686256 | ATAATACATGCCAAGAGGGTAGG | 675 |
| hs132684973 | GTTTCCCACACACCTCTGGAAGG | 676 |
| hs132686841 | ATCGTCTTCCAAGAGTGTGTGGG | 677 |
| hs132689946 | ACAGTTAGCCATCCTTAGAGTGG | 678 |
| hs132686965 | TCCGGTTGCCGTGAATAGTGAGG | 679 |
| hs132687065 | TCCATGTCCTAGCTCGAGGGCGG | 680 |
| hs132688507 | ATCATCGCTGAGAAAAGGCAAGG | 681 |
| hs132684054 | GTATCTTGACCCTCTCTCAGTGG | 682 |
| hs132689830 | TTGTCAAGTTACCATGCCCGTGG | 683 |
| hs132691378 | TCATGTAGCAAGGAGTGCAGAGG | 684 |
| hs132685987 | ACATCGTTAGAAGACAGTAAAGG | 685 |
| hs132683620 | CATTTATACAGAAGACCCACGGG | 686 |
| hs132692259 | TTTGAATGTCTGGTATTGAGTGG | 687 |
| hs132685625 | GTAATTAAAAGTGCTTTAGGGGG | 688 |
| hs132690006 | CACAGAAATAGCAATATGGGAGG | 689 |
| hs132689932 | AATAATGTCAGAGATCGTGATGG | 690 |
| hs132685918 | TCATGTATCAGTAGCTTTGGGGG | 691 |
| hs132686386 | TGTTCCATAAGTATTGGGCACGG | 692 |
| hs132683843 | CTGATGTTTATACAATGCCAAGG | 693 |
| hs132683977 | TTTGAACACATCGAGGCAGGTGG | 694 |
| hs132691989 | ACTGACCTCTAAAACTTAGGGGG | 695 |
| hs132689465 | CCATTTCCCAGGGAACCAGTCGG | 696 |
| hs132692517 | GCTCTTCAAAGCAACATCTGTGG | 697 |
| hs132683959 | TTAATGTTAGTTGTTTGCCCCGG | 698 |
| hs132689723 | GCCTGTGCATACCAGATCTGTGG | 699 |
| hs132683458 | AATGATACTGGGCAAACTGAGGG | 700 |
| hs132691103 | AAATATGCATGATATTTGTGGGG | 701 |
| hs132691922 | GAATGACTCTCAGCCTAGGGTGG | 702 |
| hs132687669 | TATTGTTAAACATCAATAGAGGG | 703 |
| hs132688177 | GGGACCGAATAGACAATGCAAGG | 704 |
| hs132684217 | GTAACACGGGTGACAAATGTGGG | 705 |
| hs132683942 | GTCTTCTATGGCAAACCATGGGG | 706 |
| hs132683984 | TTGACACCCGTAGCCTATGGGGG | 707 |
| hs132686058 | TGACTGGACTATTCTTACAAGGG | 708 |
| hs132693379 | CCCTCCTAGGAATATATCTGTGG | 709 |
| hs132684980 | TTTCGTTGACCCTTAACTAGGGG | 710 |
| hs132687994 | CTTGAAAATTCAAATCCACAGGG | 711 |
| hs132685637 | TAGAGGGAGATAAAGGAAGGGGG | 712 |
| hs132684146 | CTAAACACTGAGCCATGAGGTGG | 713 |
| hs132686414 | GTAGATAATTATAACTCAGGAGG | 714 |
| hs132692738 | GTGATTAGAAGGCATTACATGGG | 715 |
| hs132688698 | GGTACCTGAGGGATCTGTGAGGG | 716 |
| hs132686979 | ACTGAGGTTACTGATTAGAAGGG | 717 |
| hs132685985 | GATCAGTGAAATTTACATTGGGG | 718 |
| hs132688255 | AGGCAAAGTGTCCTCTAGGGTGG | 719 |
| hs132691999 | ACAGATGAATTCAACACACCCGG | 720 |
| hs132691459 | CCAAATCCCCTACATTCTCAGGG | 721 |
| hs132685585 | TCAGGGATAAAATTCTGTAGAGG | 722 |
| hs132693564 | GGAGTGATAACATATTGCACTGG | 723 |
| hs132692047 | TCAATATTCTAGACATAGTGGGG | 724 |
| hs132685401 | ATAGTGCTGGGAATGCCAGAGGG | 725 |
| hs132685570 | GTCACAACCCTAGAAATGGGAGG | 726 |
| hs132693261 | AGGCTAATACTGGTCTGTGGCGG | 727 |
| hs132686997 | ATTTTTAAAGGTGTGTCTGGAGG | 728 |
| hs132687573 | TATATCAGTAACTTTAAATGGGG | 729 |
| hs132692230 | TATTGCACAGTTACATCTCAGGG | 730 |
| hs132689407 | GTACAAATGTAGGTATGAGGAGG | 731 |
| hs132686967 | ACTATTCACGGCAACCGGAGTGG | 732 |
| hs132692664 | TTCATCCTAAGGTCAATAGGAGG | 733 |
| hs132693001 | TCAAAAGAGAAAGAATCACTTGG | 734 |
| hs132683536 | TCTAGGGAGTGCATGAACAAAGG | 735 |
| hs132685481 | CCAAATAATAAGTAGAACCAGGG | 736 |
| hs132687593 | ACAACTTTTGACCAGTAGCGAGG | 737 |
| hs132684098 | GACCAACCGCTTCACCACACCGG | 738 |
| hs132690878 | GCAAGCACATCATTACTGAAAGG | 739 |
| hs132688721 | TGCGTCAAGATGCATCATGAGGG | 740 |
| hs132690324 | TGAATGCTATAGGAATAAAGAGG | 741 |
| hs132690348 | CAGCGTGAAAAAATCCTACAGGG | 742 |
| hs132683506 | ACCACTAACGTCACCTCAGGCGG | 743 |
| hs132692925 | AGTTGATCATCACATGCTTGTGG | 744 |
| hs132685131 | TGGCACCAAAGATTATAAGCCGG | 745 |
| hs132684414 | ATAGGCAGTTCAACTAAGGGCGG | 746 |
| hs132690640 | TGACACCAGCTGGACATCAGAGG | 747 |
| hs132687912 | AAACGCTGTACTCATCTAGAGGG | 748 |
| hs132685133 | TTATAATCTTTGGTGCCAGTAGG | 749 |
| hs132688912 | AAGAGTAATCATATCCCTAAGGG | 750 |
| hs132690785 | AAATGGATTGAATTTAGAGGAGG | 751 |
| hs132685235 | GATGGCCTGTGAATAGCTGTCGG | 752 |
| hs132684639 | TCTGCGTGCATTTTTTCCAGCGG | 753 |
| hs132686512 | CAGTTTTCTACCATAGACAAGGG | 754 |
| hs132692630 | TAGATCTGAAAAGTAAGGAACGG | 755 |
| hs132693339 | CATTACCACAATAGTCCAGTGGG | 756 |
| hs132688080 | GTAACATTAATTAAGAACCCTGG | 757 |
| hs132691988 | TACTGACCTCTAAAACTTAGGGG | 758 |
| hs132688627 | GCATGCAAGAGAATCAATAGAGG | 759 |
| hs132687473 | GAAAAGGAAACACTACTCCAAGG | 760 |
| hs132685584 | TCACCACTCATATACTCTCAGGG | 761 |
| hs132684401 | GTCGGTGCTTGAGAAGGTCATGG | 762 |
| hs132685338 | AGAAGGAGACTGATTTCCCAAGG | 763 |
| hs132693067 | AGCATGAGTTTCCTTCCAAGTGG | 764 |
| hs132690743 | GAGTATTAACAATAACACAATGG | 765 |
| hs132689479 | GGCATCTGAGTGAAATATGTGGG | 766 |
| hs132685402 | CATAGTGCTGGGAATGCCAGAGG | 767 |
| hs132688753 | GTGACCAAGCCTCTTGAGAGAGG | 768 |
| hs132691200 | CCTCAGTCTAGAGAACTGGCTGG | 769 |
| hs132686276 | GGAGTCATTAACTTTAGTGGAGG | 770 |
| hs132687522 | CCAACACCTAATTAATGACAGGG | 771 |
| hs132691506 | ACCTGTTGTAAAATATCGAGAGG | 772 |
| hs132687571 | TATACTTAATCTGTATGCCACGG | 773 |
| hs132691788 | TACTTTTTAAATTCTCAGTGAGG | 774 |
| hs132691435 | GCAGAGAGAAGACAGATAGGTGG | 775 |
| hs132692255 | GAGATGCAGGAATATTTGCAAGG | 776 |
| hs132689213 | GCTGCCACTACAAAAGAATGAGG | 777 |
| hs132691583 | GAAGTGCTTTTTATCCCAGACGG | 778 |
| hs132687774 | ATAAGTAAGGAATCAAGGTGAGG | 779 |
| hs132691332 | GTAAATGCAATAATGATATGGGG | 780 |
| hs132685672 | ACAATATTTAGCAATTACACAGG | 781 |
| hs132689601 | TCTATTTGTTAGATGACACAGGG | 782 |
| hs132690133 | TAAATATGAAAACCATACATGGG | 783 |
| hs132685211 | ACTATGCCCGTACATAAACCTGG | 784 |
| hs132693954 | AATTACTAATGAAAGTTGGGAGG | 785 |
| hs132688798 | ATCCTTAACTTGGAGTCCAGTGG | 786 |
| hs132685851 | GCAAAGCTCCTATTGTGGGAAGG | 787 |
| hs132684750 | ACAAAGTCTATTGTGATCAGTGG | 788 |
| hs132685657 | CCAATTCCCCTCCTCTAGGGAGG | 789 |
| hs132685991 | GCAGACCAGAGTAAATAACTAGG | 790 |
| hs132694169 | ATTCTTATTGTATTTCCATGAGG | 791 |
| hs132685947 | TTTGCTAAGGCAGCTCAGAAGGG | 792 |
| hs132688939 | GAGAGCTTGACACTGCATGCAGG | 793 |
| hs132689784 | GCCAAATTGATGCAAAGTGGTGG | 794 |
| hs132688462 | TCTCAGCTAAGGGACGACATAGG | 795 |
| hs132689501 | GCATGTTTATCAGTGTTACACGG | 796 |
| hs132687736 | GGAAATGGTGGGTTTTCCAGTGG | 797 |
| hs132693803 | ACTACATACTGAAGGATTTGGGG | 798 |
| hs132686726 | TAGGGCCCTAGAAACCTCCAAGG | 799 |
| hs132688934 | GGGAATCCTTCTGATACAGAAGG | 800 |
| hs132684437 | CTCTAGGATTGGAATAACACAGG | 801 |
| hs132691143 | TAATCAGCTGTCTATACCAGTGG | 802 |
| hs132691919 | TCGTCACCTACTCCCTTGAAAGG | 803 |
| hs132693491 | AGAGTACAAGAAGTAATCAAGGG | 804 |
| hs132691809 | ATACTAATAGAAGACAAACTAGG | 805 |
| hs132693845 | CTATGGTTAGTAATATTAGAGGG | 806 |
| hs132692064 | TCTGTAATAAAAAGTATGGGTGG | 807 |
| hs132684956 | GCAGAAATAATTCCATGATGCGG | 808 |
| hs132688938 | AGAAGCTATCTGATTACTGGAGG | 809 |
| hs132693799 | TGCAAAGTCTGCCAGCACACAGG | 810 |
| hs132692951 | TTGTGGTTATCTGATACAGGTGG | 811 |
| hs132684932 | AGTATGCAGATTTTAAGTGGTGG | 812 |
| hs132692441 | CGTTGTGAAAGAAATTTCCAGGG | 813 |
| hs132686295 | ATATGTAACACATTAAAATGTGG | 814 |
| hs132691930 | GTTTTAAACAAGACTTTAGGCGG | 815 |
| hs132689761 | TATTGTTGGAGGTTTCCTGAAGG | 816 |
| hs132686649 | CATTGTATATATAATAGAGAAGG | 817 |
| hs132689292 | TTTTGAGTGACTTTTTGTAGAGG | 818 |
| hs132694253 | CTCTTCAGAAAGATGAGCAGGGG | 819 |
| hs132688813 | TACTCAGATTCAAAAGAGAGAGG | 820 |
| hs132685911 | TGTCTGATAAAACCATAACAAGG | 821 |
| hs132690527 | TCACGCCTTGGCCTAAACAAGGG | 822 |
| hs132693756 | AATGCAGGTCAAAAGTGTGTGGG | 823 |
| hs132690038 | GGGAAATTGGAACAACAGGAAGG | 824 |
| hs132689013 | ACATAGCATTTGTGAGTGAAGGG | 825 |
| hs132688442 | GAAGTGTGAATAAAAGTTAGGGG | 826 |
| hs132691719 | CCCAACAGTCTAATAACGATAGG | 827 |
| hs132692653 | CAGTGAGAAGAATATAGAGAGGG | 828 |
| hs132685289 | TGCATTGGCAGTGACAGCGGAGG | 829 |
| hs132683588 | TGACCTACCTGAGAAAGAAGTGG | 830 |
| hs132685151 | GTCATATAGTGAGATGAGGATGG | 831 |
| hs132692466 | CAGCTACTCTACACGCAGCATGG | 832 |
| hs132694347 | GTAGCACTGTGTACACTGAAAGG | 833 |
| hs132689470 | AGTCGGGTCACAGGAAGAGTGGG | 834 |
| hs132684994 | TTATTGTCCATATAAATAGACGG | 835 |
| hs132692907 | TTTAATCAAAAATTTCAACCAGG | 836 |
| hs132690049 | TAAGTCCCTTCTTTAAACTGTGG | 837 |
| hs132691734 | AAGTCTAAGAACAAATCAGAAGG | 838 |
| hs132684810 | ACCATGCCCTCTACTGATGTGGG | 839 |
| hs132693300 | TTTGCATAAAATCTACCAGTAGG | 840 |
| hs132684478 | TTTAAAGTGGCACAAACAGAGGG | 841 |
| hs132684289 | ACGATGGAGTGCAGAATATGGGG | 842 |
| hs132683621 | TCATTTATACAGAAGACCCACGG | 843 |
| hs132686532 | ATTTATCAGTAGTTTAGGCGTGG | 844 |
| hs132687064 | AGTTCCATGTCCTAGCTCGAGGG | 845 |
| hs132684304 | AATTCTGCCTGAATACCAGAAGG | 846 |
| hs132690025 | TAAATATGCAACTCATGGAGTGG | 847 |
| hs132684072 | TGGAGGGCGACGCGCAGAGGCGG | 848 |
| hs132690639 | GACACCAGCTGGACATCAGAGGG | 849 |
| hs132686539 | AAGCAATTATGTCGATAACTGGG | 850 |
| hs132694019 | AGCCCACCAACTGAAATGAAAGG | 851 |
| hs132689218 | TCTTTTGTAGTGGCAGCCAGAGG | 852 |
| hs132684480 | GGAGGCACCAGTGTTTAAAGTGG | 853 |
| hs132688157 | ATGCCAACCTGAAGAACGTGAGG | 854 |
| hs132692858 | CATCTACCATTATTTCCACAGGG | 855 |
| hs132694155 | CTTAGGAATATGGAGCAATGAGG | 856 |
| hs132686982 | AGTTATTTCTAGAGGAACTGAGG | 857 |
| hs132686368 | ACACATCCTTGAAGAATGAGAGG | 858 |
| hs132691611 | TAAACTTTCACTCAAACCCATGG | 859 |
| hs132684542 | ATATGGCAAACTAAAGAAAGGGG | 860 |
| hs132684479 | GTTTAAAGTGGCACAAACAGAGG | 861 |
| hs132689771 | ACAGGAGCTCTAAGTCCAGTGGG | 862 |
| hs132690156 | TCCCCCAGTTCCCATTAGTGAGG | 863 |
| hs132690945 | ATTGTCCATTTACTGGACAATGG | 864 |
| hs132689271 | TTGTTGAATGCTGATCCAGAAGG | 865 |
| hs132693794 | CAAAGAGCGTAGGAATTCAGTGG | 866 |
| hs132683420 | GGTGGCTTTAGGTTAACTAAGGG | 867 |
| hs132685083 | AATGTTACATGAAAAATGAACGG | 868 |
| hs132691177 | GGATTAAATCATGAACAGGAAGG | 869 |
| hs132689113 | TGGTCTCTATATCAGTCGATGGG | 870 |
| hs132689037 | CATAAATTAATGCAAACGTGTGG | 871 |
| hs132686059 | GACTGGACTATTCTTACAAGGGG | 872 |
| hs132688833 | GTTGGAAAATAAGAGGGCAGAGG | 873 |
| hs132687820 | ATCAACCAGTAATTTCTGAAGGG | 874 |
| hs132683477 | AGAAAATGTATCTTTAAAAGGGG | 875 |
| hs132693864 | TTAGGAAGATTCTATGCACAGGG | 876 |
| hs132691409 | ACAATAGTTTACATTTACTGAGG | 877 |
| hs132684869 | GGCACAGCTACCAGGAAAAGGGG | 878 |
| hs132686329 | GGTAGCCAATTATGGCCCACAGG | 879 |
| hs132688319 | ATTGATTTGAATATACACTTCGG | 880 |
| hs132689361 | AGATGAGAAGATAGTCTTTGGGG | 881 |
| hs132694386 | CGAGAAATCATAGACCAGCTGGG | 882 |
| hs132684890 | TCTCTCTCAAAAGTACTGCAAGG | 883 |
| hs132690398 | ACTACTGTATAGGAACTACATGG | 884 |
| hs132690811 | ATATTTAAAATAAAAAGGGGAGG | 885 |
| hs132691084 | AATTTAGATCCCTTTCCAAGTGG | 886 |
| hs132689163 | ATATTTTCCACAGTAATCAGTGG | 887 |
| hs132690182 | TTGGGAATTTCAGTAGGCAACGG | 888 |
| hs132683472 | TTTTAAATGATATTTCCTTGGGG | 889 |
| hs132693720 | TGTATCTACTTGGAACTCGTGGG | 890 |
| hs132690200 | AGACAAATGAGAATGAGGAAGGG | 891 |
| hs132683914 | GAATTCACAAACACAGATAGGGG | 892 |
| hs132684972 | CACACCTCTGGAAGGAACACAGG | 893 |
| hs132692189 | GTGACTGATATGAATTCAAGGGG | 894 |
| hs132684435 | GTGTTATTCCAATCCTAGAGTGG | 895 |
| hs132694390 | ATATATCAATATCTCCTGCAGGG | 896 |
| hs132691699 | TTATTTTCTGGTTGGCACTGAGG | 897 |
| hs132691505 | TATTTTACAACAGGTATAAGCGG | 898 |
| hs132685065 | ACAGTCCTAGACACCCATAGAGG | 899 |
| hs132683743 | ATGCTAATAGATTATCCAAGTGG | 900 |
| hs132685754 | TAGACTATATTTGTAGCTGTGGG | 901 |
| hs132684881 | CATTCTCAAAAATTATATCGAGG | 902 |
| hs132689831 | GACAATGCCTGAGACTACCTGGG | 903 |
| hs132691509 | GGTCATCTAGTCTCTAATGAGGG | 904 |
| hs132689607 | GTCTAAGCTAGTACTAGGTGAGG | 905 |
| hs132690693 | TTGTTAGACCCATCTATGAGTGG | 906 |
| hs132686838 | ATATATGCTTCAATTTTGTGAGG | 907 |
| hs132691672 | GCTTATTAGGAAAATCACATTGG | 908 |
| hs132687671 | GTTAAACATCAATAGAGGGAGGG | 909 |
| hs132686457 | ACGAATTATTCATCTACCCGTGG | 910 |
| hs132683562 | GAGCAGTATGATGAAGTGCAGGG | 911 |
| hs132685549 | TGATTTAAGTGAGTGTATCAGGG | 912 |
| hs132686656 | CAGCATGGTTCAGTTGATGGTGG | 913 |
| hs132687762 | CTACTAGTAACACAATACTCAGG | 914 |
| hs132689429 | AAGCATTGTAAGGTACTACATGG | 915 |
| hs132693141 | TTTCTAAGAACTAGGCCTTGGGG | 916 |
| hs132690769 | GAAAGGAGAGAAGCTTTGAGAGG | 917 |
| hs132685158 | ATTTTACACCAGGTCGAAGGAGG | 918 |
| hs132687191 | ATGCCCAGAGCCATAAGGAGTGG | 919 |
| hs132690454 | AACCTTATATGTAGTGACCATGG | 920 |
| hs132687932 | TAACAAAAACTAGAACAAAGTGG | 921 |
| hs132686087 | ACAGGGACCATCCTATCCATAGG | 922 |
| hs132687511 | GGAAGAAGCTGGGAGTCCAAAGG | 923 |
| hs132683409 | TTTCTGCTACCAGTAGAAGTGGG | 924 |
| hs132687236 | TGATTTTAAAGAAATAGTAGTGG | 925 |
| hs132686122 | TCCGTGAGCCTAAAGAACAATGG | 926 |
| hs132683923 | TATACAAAACAAAACATGAGAGG | 927 |
| hs132689664 | ACTAAAATAGCTAACTTCAGAGG | 928 |
| hs132693429 | CCTACACTATGCAAGTGGGTTGG | 929 |
| hs132693815 | AGTAACACAAAGCAAAAATGTGG | 930 |
| hs132687643 | TGTCACTTTCATTTATTCGGGGG | 931 |
| hs132685919 | ATGACAGGTTTATATTCTGGAGG | 932 |
| hs132688371 | ATTGGCTTAGAAAACATGAGTGG | 933 |
| hs132688559 | TATACTTATATTCAAGCACAGGG | 934 |
| hs132690907 | AGAGTTCCCAGGACGCAGGAAGG | 935 |
| hs132684214 | AAAAGGCTGCCAGAGTAACACGG | 936 |
| hs132684464 | AAAGTAAGCAAAATTGAGGAGGG | 937 |
| hs132684352 | ACTCTTGATTTCACTGACAGAGG | 938 |
| hs132691373 | CATGAATAAATACATTCAAGAGG | 939 |
| hs132685233 | GTAGACCGACAGCTATTCACAGG | 940 |
| hs132685618 | TAACTCATAGTTATATAATGGGG | 941 |
| hs132691718 | CTAATAACGATAGGAAACAATGG | 942 |
| hs132684286 | ATTGCCTTCTTACCTGACGATGG | 943 |
| hs132693603 | TTTCATACACACTCATCATGGGG | 944 |
| hs132692241 | TCATTCAGAGATTATAACCAGGG | 945 |
| hs132689402 | ATTTACTGTGGCAACAGGAGAGG | 946 |
| hs132692218 | TGTCTGTGAGACAAAATGAAAGG | 947 |
| hs132691245 | TGTTGACCCTGATGATGCTGAGG | 948 |
| hs132689945 | TAGTGAGACCACTCTAAGGATGG | 949 |
| hs132688030 | CTACATAAACTCTATTCATGGGG | 950 |
| hs132690410 | GCTTTGGTGGGAAGAAGTAGAGG | 951 |
| hs132685053 | TCCCTTAAGACTTACTCTGACGG | 952 |
| hs132683657 | CACAGGGCCTGGATTATAAGGGG | 953 |
| hs132692211 | ACGCAGATTGTAATTAGAGGTGG | 954 |
| hs132686519 | GTAAGCCACAAGTGTAATGGGGG | 955 |
| hs132685879 | AATAGCAAGGAGTAGATACAGGG | 956 |
| hs132691274 | CCTTTAAGTAAGACAGCCAGGGG | 957 |
| hs132692017 | TTATACAAATAAATTTACAAGGG | 958 |
| hs132684024 | TCGAATGTGGATATCCCAAGAGG | 959 |
| hs132691855 | TGGGGACAACAGCAAAGTGGTGG | 960 |
| hs132685813 | ATTCAGGTTTGGGATCAGGAAGG | 961 |
| hs132694254 | GCTCTTCAGAAAGATGAGCAGGG | 962 |
| hs132688171 | GCGAAAACCAGAGCCACACTAGG | 963 |
| hs132690633 | ACTTCACATATGCCTCACTAAGG | 964 |
| hs132693858 | CATAAACTATTAGTGCCTGAGGG | 965 |
| hs132692903 | ATACATAGCCCATATGTCAAAGG | 966 |
| hs132688156 | GGAGAGCTGCAGCCTACATGAGG | 967 |
| hs132685154 | CCCTACATAATAGTATGCAAGGG | 968 |
| hs132688065 | ATAGTGTGGGTCAACTGAAGGGG | 969 |
| hs132694290 | GAATGGATTGGACAGACGCTAGG | 970 |
| hs132687102 | TCTGTGACGACGATCATTGGTGG | 971 |
| hs132683383 | TTCTTGTATTAAAGACGTAGAGG | 972 |
| hs132687513 | GATCGGCAAAGGAAGAAGCTGGG | 973 |
| hs132692054 | AATACCTACATAAAAATAAGGGG | 974 |
| hs132689844 | ATAAAATATCATGATGATGGAGG | 975 |
| hs132690839 | TTAATACTGCAATATAAGCAAGG | 976 |
| hs132692071 | ACTTATAGCTACGATCCACTTGG | 977 |
| hs132692225 | TCAGGGTAATGCTTAGAGGTGGG | 978 |
| hs132689410 | TTTAGCATAGTTGCAATGTGTGG | 979 |
| hs132692787 | ATTTTGGATTCTGGAGACAGTGG | 980 |
| hs132691712 | CCAGTTTCCAAAAAAGTCTGAGG | 981 |
| hs132691613 | ATAATTAGACAATTCAGAGAAGG | 982 |
| hs132685713 | AAAAGAACCTAAAGTACACAGGG | 983 |
| hs132683416 | AATGGACATATTTTCCCAAGTGG | 984 |
| hs132686270 | ATAAATTCATAGTCAATATGGGG | 985 |
| hs132688450 | TGGTGGCTTGAGGAAATGTGGGG | 986 |
| hs132685103 | GTGCTAACATGCAGTAGAGGAGG | 987 |
| hs132685741 | TTTTTTTTCAGTCAATCTAGAGG | 988 |
| hs132692171 | AGATGTCATTGGAAAAATGGGGG | 989 |
| hs132690692 | TGTTAGACCCATCTATGAGTGGG | 990 |
| hs132688422 | ACAGGATTGTTGATTGCCTGTGG | 991 |
| hs132692335 | AGACTATTGCCTTCATAGCGAGG | 992 |
| hs132686484 | GATGAATGATCACATTAGGAAGG | 993 |
| hs132685068 | ACAAGTGCTAGTGCGTGAAGTGG | 994 |
| hs132694335 | TCAGTAAATTAATTAGCAAGTGG | 995 |
| hs132690094 | TGCCTTTGATGAAAATACGTGGG | 996 |
| hs132692460 | CCTCCACCTTGAGGGCTCAGAGG | 997 |
| hs132686128 | AAAAAAAGAAACTTCCAAAGAGG | 998 |
| hs132690609 | TCCATAGACAATGAAGCCCATGG | 999 |
| hs132690536 | TGGCAATTAAATGTATCTCAGGG | 1000 |
| hs132686970 | ACACTCCCCTCTCACAGCAATGG | 1001 |
| hs132686255 | GTGTATAATACATGCCAAGAGGG | 1002 |
| hs132693432 | CCAACCCACTTGCATAGTGTAGG | 1003 |
| hs132685458 | GCAAGTCCAAAGGCATGTGATGG | 1004 |
| hs132693873 | AATTGTTTATGACTGTGGGATGG | 1005 |
| hs132685488 | ATACCTCAAAGGAAAATGGCCGG | 1006 |
| hs132693313 | GTAGAAGTACTATATATAGGAGG | 1007 |
| hs132691037 | ACTTTGCCTACACAAAAAGCTGG | 1008 |
| hs132690447 | AACCAACTTGAGAGAGAAGGCGG | 1009 |
| hs132687164 | TCAAGGGCTTGTGAAGATGAGGG | 1010 |
| hs132683514 | CAGAGGAGAAAAATGAGAAGAGG | 1011 |
| hs132691137 | GCAGACATATTATCTGATCAGGG | 1012 |
| hs132688218 | GACTGGCTAGACTAGGGCTAGGG | 1013 |
| hs132688520 | ACATACGGATGAATTCAGTAAGG | 1014 |
| hs132686340 | AAACAACATCTACCTGTAAGGGG | 1015 |
| hs132694098 | ATGAGAGCTTTATACTAAGACGG | 1016 |
| hs132694317 | TTTTGCATCCAAATGCTACAGGG | 1017 |
| hs132690147 | CATTTGAATAAAAGAATCTGAGG | 1018 |
| hs132688070 | TCAAATCCAGATGCTACCACAGG | 1019 |
| hs132691372 | ATGAATAAATACATTCAAGAGGG | 1020 |
| hs132686853 | AGCAATGTCAGCGAGCTTGGTGG | 1021 |
| hs132684822 | TACATTTTTTATGAGCCCCGTGG | 1022 |
| hs132686528 | AAAGGCATTATGGTAGGGGGAGG | 1023 |
| hs132686825 | TAGTTTTAATGGCCCAAAAGGGG | 1024 |
| hs132693400 | GCCAAGTGCCAAATGGTGCAGGG | 1025 |
| hs132690631 | TTAGTGAGGCATATGTGAAGTGG | 1026 |
| hs132683764 | TCACTGAGTTGAACTTGTAGAGG | 1027 |
| hs132691385 | TCTACTGCATATAACACTGATGG | 1028 |
| hs132689448 | AGGGCAAAAGAACTGTAGACGGG | 1029 |
| hs132689937 | TAGTACTTGGCATAAACAGTAGG | 1030 |
| hs132690247 | AAGTTAAAATGGCAACACCATGG | 1031 |
| hs132687995 | CTATCTTTTCAAGATCCCTGTGG | 1032 |
| hs132693374 | ACCACAGATATATTCCTAGGAGG | 1033 |
| hs132684166 | ATTTGCAGCCTTTTGTAGAGGGG | 1034 |
| hs132688954 | ATTTACGTATATTATGCCCAAGG | 1035 |
| hs132693919 | ATGGCAGAAGAACTAAAGGCAGG | 1036 |
| hs132690541 | ATAATGATTTCTGCATTGTGTGG | 1037 |
| hs132693880 | CTGTTTGTATGGCTAGGGTGAGG | 1038 |
| hs132687286 | TTTTGGCTTCTCTCCTAGGGAGG | 1039 |
| hs132684821 | ACATTTTTTATGAGCCCCGTGGG | 1040 |
| hs132694151 | AAGACTATAAAACTGAGGAAAGG | 1041 |
| hs132683849 | AATGCAATTACAATTATGCAAGG | 1042 |
| hs132686218 | TCTCTGTTGGAAGTGAATAGGGG | 1043 |
| hs132688732 | CAAAGGAAATAAAAGTATGGCGG | 1044 |
| hs132691048 | TCTGAGGTGAGAAGCTAGTGAGG | 1045 |
| hs132692274 | CTGCCCTCTGTAAATATGAGAGG | 1046 |
| hs132692222 | TATTCCAAGGGCCTACCGCAAGG | 1047 |
| hs132691095 | AGAACCCAGCTAAGAGAGGGAGG | 1048 |
| hs132690810 | TCTATATTTAAAATAAAAAGGGG | 1049 |
| hs132693796 | CTTGAGGCTGCAAAGAGCGTAGG | 1050 |
| hs132688644 | TCAATAAATTCGTAGCCCTAGGG | 1051 |
| hs132691918 | AAGGGAGTAGGTGACGAAGATGG | 1052 |
| hs132692093 | AGGTGTGTCTCAGGGTAGAGAGG | 1053 |
| hs132692973 | GGCCTTCCTTAGAATCTGCACGG | 1054 |
| hs132689197 | TTGAGAGTGATTGTTACCAAAGG | 1055 |
| hs132689346 | AAATATATATTCAATACCATAGG | 1056 |
| hs132687765 | CTCACAGTCGCCCCTATGAGGGG | 1057 |
| hs132686367 | GGACTGCTGAGTTTCTGTAGAGG | 1058 |
| hs132683824 | GAGTGGTTCCTTCATGACATGGG | 1059 |
| hs132689247 | AATGAAAGTTGTAACAGGAGAGG | 1060 |
| hs132691696 | TGACCACTAACTAAATCTGAAGG | 1061 |
| hs132691013 | GCCTAATCTAATTCATAGTGAGG | 1062 |
| hs132690102 | AGTGCTGACTTAAAGGGCAAGGG | 1063 |
| hs132690415 | AGGCAGCGATAAAAAAAACCTGG | 1064 |
| hs132685941 | TTGCAGTTTGTGTGGTTACGTGG | 1065 |
| hs132683369 | GAAAGTACGCATGCACATGTAGG | 1066 |
| hs132693142 | CTAGCCTGAGCCACACACAATGG | 1067 |
| hs132691008 | TGGGTCCCCATTAATTAGCAAGG | 1068 |
| hs132688306 | AGTAAGCCTTTGGATGGGGAAGG | 1069 |
| hs132684861 | ACTCCCAAAGATATATAAGTCGG | 1070 |
| hs132693488 | CATTTACTAGATAACAAACATGG | 1071 |
| hs132690249 | TTTTATAGCATGAAACCCCAAGG | 1072 |
| hs132687882 | TTTGCAAAACAATTTAAGAGGGG | 1073 |
| hs132693257 | ACAACGTGCTATTAGCAAGTAGG | 1074 |
| hs132686521 | GGGGGGATCTCCTAAACGAAAGG | 1075 |
| hs132689526 | TCCCTGACAACACGAAAGCATGG | 1076 |
| hs132688928 | ATCATCTCAAAGAACTACATGGG | 1077 |
| hs132693198 | GTAGGAAGGAAGGCAAACTAGGG | 1078 |
| hs132685643 | GGAATAAAATAAGAATGCGAAGG | 1079 |
| hs132689550 | CCTTACATACATCACGGAGTTGG | 1080 |
| hs132683894 | GAGCATCATGACCTTACAAGAGG | 1081 |
| hs132693522 | TTTCAAAGAAGGCTGACCACAGG | 1082 |
| hs132686879 | AGCTAGTTTTTTTTTCAGGGGGG | 1083 |
| hs132692889 | TGTCTTCTAGGGTACACTGTAGG | 1084 |
| hs132683625 | TCATTCTTTTGAGCATGAGGAGG | 1085 |
| hs132685063 | ACTATCAGCAATTAGTGAGAGGG | 1086 |
| hs132687268 | AGTACCTTACAGTGCAAGCAGGG | 1087 |
| hs132687512 | CGATCAGCTTTAAGGGACAGTGG | 1088 |
| hs132687637 | TATCTTTGATTGAGGGAAGGAGG | 1089 |
| hs132683670 | TGTAATCCAGAATGGCCCATAGG | 1090 |
| hs132689087 | AATCATTATTATCAACAGAAGGG | 1091 |
| hs132684332 | TTTTTTTACAAGGGAATGTGAGG | 1092 |
| hs132690600 | TAGAATTCAAGCAATTTGGAGGG | 1093 |
| hs132688260 | CTATTATTCAAAAGCCAGCAAGG | 1094 |
| hs132688240 | TCTTATAATAGGTTATTGGAAGG | 1095 |
| hs132684151 | GGGCTGCCAGGTAAATTGGAAGG | 1096 |
| hs132684413 | AGAATAGGCAGTTCAACTAAGGG | 1097 |
| hs132688927 | TGATCGATCTATGTTTTGGGAGG | 1098 |
| hs132692453 | GTGTGTCTATGAAAAGGAGTAGG | 1099 |
| hs132693397 | AGAACCCTACGCATAAGAGCAGG | 1100 |
| hs132685497 | TGTTCAGTTTTGGTGCAAGGTGG | 1101 |
| hs132689274 | TGTGCTAGCATAATCTCACTAGG | 1102 |
| hs132689793 | AAAAAGAGCCCCAATTTGTGGGG | 1103 |
| hs132690862 | GCTATTAGGTCTATGTGAGGTGG | 1104 |
| hs132683624 | AATCCTACTAATTATTAACATGG | 1105 |
| hs132691217 | AATGTAGTACATATCCCCATTGG | 1106 |
| hs132684224 | ATTTCTTAAAATCCTCACGAAGG | 1107 |
| hs132687758 | AGGTGAGAGCTGGCAAACACAGG | 1108 |
| hs132689720 | TTATTATAAAAGCAGGTCAGAGG | 1109 |
| hs132692815 | GGAAGGATCTGCGCTAGGTGTGG | 1110 |
| hs132684394 | GCATGTAGATTAGCCTCACAGGG | 1111 |
| hs132691005 | AGATTAGCGCCATCTCCCATGGG | 1112 |
| hs132694186 | TGAACATAGGGATATGAGAAGGG | 1113 |
| hs132689759 | TTCCTGAAGGAGACTTAGAACGG | 1114 |
| hs132686171 | TCCAAACTGATAAGTCCTCAGGG | 1115 |
| hs132685093 | TTTTAATCAAGTATATTCGGTGG | 1116 |
| hs132687606 | ATACACCTAATTGCTCTCTAGGG | 1117 |
| hs132684650 | TTTCAACCTCTAGCCTTCAAGGG | 1118 |
| hs132685025 | GCATAAGAGGACACTGCACATGG | 1119 |
| hs132685735 | CACATGATTGAGTATCTTAGGGG | 1120 |
| hs132691014 | GCTGTCCTTGCTAATTAATGGGG | 1121 |
| hs132686049 | CATTGTTGACTCAAGGACACTGG | 1122 |
| hs132694389 | GAGTTGTTGAAAAACCCTGCAGG | 1123 |
| hs132693131 | CTGTGCATAAACACTCAACCAGG | 1124 |
| hs132689188 | ACGCAAATTCTAGTTTAAAGCGG | 1125 |
| hs132690372 | ACACTCCACAAGTCAAAGATGGG | 1126 |
| hs132684239 | CCTGTTTGTTTCTTTCACTGTGG | 1127 |
| hs132690660 | ATCCAAGACTGAAATGGTAGGGG | 1128 |
| hs132692126 | CTTCTCAAGTGTCTAAGCTGAGG | 1129 |
| hs132691117 | AACATGTCATCTGGTAACCATGG | 1130 |
| hs132688000 | TTAGAAATAAACTAGCCAGAAGG | 1131 |
| hs132684296 | TATGGTTCTGAATTAGCACTCGG | 1132 |
| hs132687272 | AGGGAGGCATTATTGCTCAATGG | 1133 |
| hs132683596 | AATTCTGTGATTAAGTGGCAGGG | 1134 |
| hs132693427 | TAGACCCTACACTATGCAAGTGG | 1135 |
| hs132690161 | TCCTCACTAATGGGAACTGGGGG | 1136 |
| hs132685064 | AACTATCAGCAATTAGTGAGAGG | 1137 |
| hs132689622 | AGTTCACCTACATCTAAAGAAGG | 1138 |
| hs132693144 | CTGAGCCACACACAATGGATAGG | 1139 |
| hs132693110 | GGGTAATAGGAATATGGGATGGG | 1140 |
| hs132693081 | ATCCACACACTATTTTGAAGAGG | 1141 |
| hs132690899 | GGGAAAAAACGGATTGGAGTGGG | 1142 |
| hs132685594 | ACATATTTTAGTCAAATCAGAGG | 1143 |
| hs132683464 | GTGAAAGCATTTAAAAACTGGGG | 1144 |
| hs132686805 | GATATGTTTAATCAAAAACATGG | 1145 |
| hs132689143 | AAGTGTGGAAAGCACTGGGTTGG | 1146 |
| hs132687342 | AGAATGATCTGCTTAACACTTGG | 1147 |
| hs132694351 | TTTAGCTCCTCAGGCTATGGAGG | 1148 |
| hs132692816 | ATCCTTCCTCTCATAAATGAGGG | 1149 |
| hs132685101 | GTACAAATATTACATTAACATGG | 1150 |
| hs132683611 | GCATGTGTTAGATACAATGCAGG | 1151 |
| hs132692749 | TTAGGAGTAATTACCACGGTGGG | 1152 |
| hs132687336 | CGTGAGTCAGTAACCCCTGAAGG | 1153 |
| hs132694101 | GGAAAATATAAGTCAGAGGGAGG | 1154 |
| hs132686211 | ACATTATGAGGCAAATAATGAGG | 1155 |
| hs132690971 | GAATACTGGCCTTTACTGGAGGG | 1156 |
| hs132690653 | GCAAAATGGGTGTGGTAGCGAGG | 1157 |
| hs132693959 | AATTACTAATGAATGTTGGGAGG | 1158 |
| hs132685757 | ATTTCTTTAGGAGATACTCATGG | 1159 |
| hs132693655 | CTCCATATATTCTTTCAGTGTGG | 1160 |
| hs132691128 | TGTAACAAGAAACAAAGCTAAGG | 1161 |
| hs132691091 | AATGCTGCATGTCATATACCTGG | 1162 |
| hs132686455 | ATAAACTGCAGACACTTCCACGG | 1163 |
| hs132687846 | GCTAAAAGCCAGTGAGAAGACGG | 1164 |
| hs132688071 | AATCCAGATGCTACCACAGGAGG | 1165 |
| hs132687037 | TGAAAATTGAGAGTAGGGAACGG | 1166 |
| hs132686018 | AGCCCTTCTCTTAAGTAGCACGG | 1167 |
| hs132686856 | TGAAGGTTCAATAAATAGGTCGG | 1168 |
| hs132692425 | CCTCTGACTGTCCATGAGAGAGG | 1169 |
| hs132686089 | AGCTTCCTAGTGGCCACACAGGG | 1170 |
| hs132685308 | AAAAGCTTCACTGATCAAGATGG | 1171 |
| hs132691687 | CCATATTAGAAGCAAGTCCAAGG | 1172 |
| hs132684472 | ACTGATCGTGTTGCTGACAAAGG | 1173 |
| hs132687249 | CCTAAATATACAGTACATCAGGG | 1174 |
| hs132691864 | AACTTGGAAATAATTTTTAGGGG | 1175 |
| hs132687226 | ATAATCACTCACATTTCCCAAGG | 1176 |
| hs132690472 | AAAATAACTTTGGTAGACAGTGG | 1177 |
| hs132690519 | ATTGTACCTCACAGCATAGAGGG | 1178 |
| hs132683474 | AACATATTAACTAGCCCCCAAGG | 1179 |
| hs132688271 | TTTACTAGATCATCACCGGCAGG | 1180 |
| hs132684196 | ACACAGCACACACGAATGCATGG | 1181 |
| hs132687426 | TCTCTTTTTATAGTGGTGGGGGG | 1182 |
| hs132691684 | ACTCCATCCATTAGTAATCACGG | 1183 |
| hs132684813 | ATAATACCCACATCAGTAGAGGG | 1184 |
| hs132687760 | AGTATTGTGTTACTAGTAGAGGG | 1185 |
| hs132686881 | ACAGCTAGTTTTTTTTTCAGGGG | 1186 |
| hs132685726 | AGAAAAATGAAGTAGAGCAAAGG | 1187 |
| hs132688353 | AATAGACTGCTATCCTGCCATGG | 1188 |
| hs132692814 | TGAGAGGAAGGATCTGCGCTAGG | 1189 |
| hs132689511 | TCAATGGAGATTGGTGAGAGGGG | 1190 |
| hs132688646 | AAGTAAGCTGTCATTACCCAAGG | 1191 |
| hs132691449 | GGGAGGACTAGTTCTTCCTAAGG | 1192 |
| hs132687969 | AATGTACAAAAAAAATACAATGG | 1193 |
| hs132691051 | TTTTTGCTAGGAGAGAAAAGTGG | 1194 |
| hs132689127 | TGATGTGGTGGATAACAGCAAGG | 1195 |
| hs132688023 | ATTGATAGCAAGATTTCAAGGGG | 1196 |
| hs132686469 | AATTACTCCAAGAAATGTGTAGG | 1197 |
| hs132688366 | GTAGCTAAAAAGTCTTGTAGAGG | 1198 |
| hs132693537 | GCATAATTCTCAAAAAGAGGGGG | 1199 |
| hs132693126 | AGAACTAATCCTTTTCACCATGG | 1200 |
| hs132692111 | TAGAGCCATGCAAATGAGAGTGG | 1201 |
| hs132684183 | GATTCCCAAAAGTAAGTGAAGGG | 1202 |
| hs132686764 | ATTCGGAACACAGTTGAGAGAGG | 1203 |
| hs132688589 | GAGTATGGCTAATAATTCAAAGG | 1204 |
| hs132686931 | AGTCATCCAAATATATCCCAGGG | 1205 |
| hs132683429 | CATGTGCATATTAATAAGAGAGG | 1206 |
| hs132688854 | CCCTCAATCCCAATTCTGCAGGG | 1207 |
| hs132691742 | AGTGGAGAAAGGCATCTCTAGGG | 1208 |
| hs132687320 | AAAAGCTTAAAGCTAACGGAGGG | 1209 |
| hs132686633 | GCAGTAGTAGTAGTTGTACTGGG | 1210 |
| hs132685288 | CTGTGCATTGGCAGTGACAGCGG | 1211 |
| hs132683935 | GTTTAAATGCACAATGCCAAGGG | 1212 |
| hs132686629 | GTCAAAGTAGCATCTTGCTGTGG | 1213 |
| hs132688012 | AAATATCCATCTTTGTGGCAGGG | 1214 |
| hs132686692 | GATACTGCATGGAACTCGGGTGG | 1215 |
| hs132690478 | ATGCAGCAGCTGAATTCTGAAGG | 1216 |
| hs132689245 | GTTGTAACAGGAGAGGAGCTGGG | 1217 |
| hs132687592 | TCACACTGAGAGTACCGCTCAGG | 1218 |
| hs132686567 | TATTATAGCTCAATTTCACCAGG | 1219 |
| hs132690369 | TTATTCTAAAATAGATATGAGGG | 1220 |
| hs132690219 | AAGTATTTCATAATCTCTAGAGG | 1221 |
| hs132684096 | TCAAGACCGGTGTGGTGAAGCGG | 1222 |
| hs132684406 | GCAAGCTGGCAGTCTTTCAAGGG | 1223 |
| hs132691752 | GAAGCTTTCCTCATAACTGTAGG | 1224 |
| hs132692219 | TAGTCACTTGCTCTTCCTTGCGG | 1225 |
| hs132683306 | CCATGCCATCCACCCAACCATGG | 1226 |
| hs132687929 | CAGGGCCATCAGCTGCCTGAAGG | 1227 |
| hs132691230 | GGCCAGTGCAGAACACACCTGGG | 1228 |
| hs132686971 | GCAATGGTTTCTTCCCTCACCGG | 1229 |
| hs132693927 | GTACAGAAGTAGATCATGGAAGG | 1230 |
| hs132686109 | AACAAGCTTACTGATATAGAGGG | 1231 |
| hs132690938 | GGCGAAAGAATCACTGAGAGTGG | 1232 |
| hs132685464 | AGCCAGGCTACGGAGCAGCTCGG | 1233 |
| hs132692277 | TTGCCTCTCATATTTACAGAGGG | 1234 |
| hs132690355 | TATCATTACAAATATATGCATGG | 1235 |
| hs132686959 | TAGCCAAGCACAGATAAAGCAGG | 1236 |
| hs132688234 | AGTTTATCTCCTAAATATGAGGG | 1237 |
| hs132683667 | AACAATTGAGGAGCCCTACAGGG | 1238 |
| hs132684373 | ACCCTCCCCACACCCCCACACGG | 1239 |
| hs132688793 | ATTTTACTAATAGTGACAAGAGG | 1240 |
| hs132694358 | TTATTTGCTTGTTACCCTAGTGG | 1241 |
| hs132692945 | TGTGCCTATCCTAATAAGCAGGG | 1242 |
| hs132693981 | AAGGTCTCTAAAGATAGCATAGG | 1243 |
| hs132690651 | AGAGAATTGCAAAATGGGTGTGG | 1244 |
| hs132689366 | ATACATCTACTGCTAATGATGGG | 1245 |
| hs132685505 | AGTTACATATTAAACAGCTAAGG | 1246 |
| hs132686404 | TAGTTCAAAGGGGGATAGGACGG | 1247 |
| hs132691546 | AAGTCTATATCAAAATATAGGGG | 1248 |
| hs132689893 | GCTAAGTTCAAGTGAAGTAGAGG | 1249 |
| hs132686566 | CTATGTAGCCGAAAGTTCCAAGG | 1250 |
| hs132684340 | AAGATGGCATTGTTAGGTGGTGG | 1251 |
| hs132684403 | AGAAGGTCATGGAAGTTAGGAGG | 1252 |
| hs132692932 | AGATATGTGCGGCTAACACAGGG | 1253 |
| hs132689954 | CCATTCTCTAGACTTGCATAGGG | 1254 |
| hs132687147 | TTACTTTGTCAGTACTTCAGGGG | 1255 |
| hs132692316 | GTACTAGCCATCAAAATTTGGGG | 1256 |
| hs132688990 | GATTCATGCAAAACTAGACTAGG | 1257 |
| hs132693973 | GCCCTCCTTTGTAACCTCTGAGG | 1258 |
| hs132693302 | CATAAAATCTACCAGTAGGTGGG | 1259 |
| hs132691345 | AGTGCAACATGATTGGACATAGG | 1260 |
| hs132686548 | GCTTTAGAACCAAAAGTCGATGG | 1261 |
| hs132693976 | GACCTCAGAGGTTACAAAGGAGG | 1262 |
| hs132687628 | ATTGCTGTGCTAGATTACACTGG | 1263 |
| hs132689786 | TCAGCTTTGTAACTCCTCAAAGG | 1264 |
| hs132687749 | TTAGTGAACGTATTTCAACTTGG | 1265 |
| hs132685172 | ACCTTAAAACAAGTTACAGAGGG | 1266 |
| hs132689066 | CTTGCATCAGTTTCTAAACGAGG | 1267 |
| hs132688608 | ATGATGCCTTGAGTGTGGCTGGG | 1268 |
| hs132690687 | AACAAAGCCCAGAAGAATGAGGG | 1269 |
| hs132687899 | GAACTCAAGGTTTTCAACAAGGG | 1270 |
| hs132685457 | ACATTGAATGGCAAGTCCAAAGG | 1271 |
| hs132687834 | AATGGCTGCATGATAATTAGTGG | 1272 |
| hs132687435 | AGCTTCCTTTTACATCCAAGAGG | 1273 |
| hs132693352 | TAGGGCTTACTTATGATGCAAGG | 1274 |
| hs132688370 | TTGGCTTAGAAAACATGAGTGGG | 1275 |
| hs132694059 | TTATGAATACAAAGAGCTAAGGG | 1276 |
| hs132693534 | AATAATTGTAGTAGAAACCAAGG | 1277 |
| hs132685219 | AATGGGAGAAATCCAAGCATAGG | 1278 |
| hs132693708 | ACATCTTGAATGAAAACAAGAGG | 1279 |
| hs132689543 | ACTCCTAACCCTTTAGCCAAAGG | 1280 |
| hs132692116 | CTTAACTCTCTGTTGTGAGGGGG | 1281 |
| hs132690073 | TTAGTAGACGCTGAAGGGCAGGG | 1282 |
| hs132684646 | CCTCTTTGAGCAGAGATCAAGGG | 1283 |
| hs132688248 | CTAAGCCCGTGTCTATGAAGTGG | 1284 |
| hs132684677 | TTTAAATACTGAAGTTCACATGG | 1285 |
| hs132683370 | TCTGCCAAAAGCTCAATATGAGG | 1286 |
| hs132687309 | TGCACAGACATCTTACAACATGG | 1287 |
| hs132686944 | AGACATTTCAAATTCTGCGGAGG | 1288 |
| hs132692496 | CTGTAACTGGAGACAAGAAGGGG | 1289 |
| hs132692206 | CATAGGGAGGAATCCCACAATGG | 1290 |
| hs132685668 | GGCTGGTTAAAAGCTTTCAGAGG | 1291 |
| hs132686194 | GCCACTGTTCATTTTAACTGGGG | 1292 |
| hs132683790 | CCATAAAAAGAATGGTACAATGG | 1293 |
| hs132690509 | AGAGGAAAGGAGGTGACCTAGGG | 1294 |
| hs132689885 | ACTAATTAATACTTGCCAGTCGG | 1295 |
| hs132685602 | GTTCAATCCTATGATAATCAGGG | 1296 |
| hs132687661 | AACTCACAAATATTGTTGCAGGG | 1297 |
| hs132685744 | GAAATGGCTTCGGAGATACATGG | 1298 |
| hs132687889 | AATCAGGACAGTAATTTGGTAGG | 1299 |
| hs132683839 | TTGTTTGAGTAACCTAGCTGAGG | 1300 |
| hs132689760 | CCTCCAACAATAGCTCAAGCAGG | 1301 |
| hs132683718 | GTGAGTTATTGTAATAGCAATGG | 1302 |
| hs132689048 | ACATTCTGTTTTTATCCCAGTGG | 1303 |
| hs132683317 | GCATCTACATACTCTAACATGGG | 1304 |
| hs132690179 | TGGAGGCCTACAACCTCTGAAGG | 1305 |
| hs132693172 | GGAACCATTAGTACTTACAATGG | 1306 |
| hs132687867 | GAACATTTAATATATAGGAATGG | 1307 |
| hs132683831 | AAACCAGTATTTTCAAAGCGTGG | 1308 |
| hs132688743 | TCGGATGCTGGCCATAAACAGGG | 1309 |
| hs132689548 | GGAGGTAGCAGCAATAAGCTGGG | 1310 |
| hs132687398 | ACAAATTCCAATAAATGATGAGG | 1311 |
| hs132687165 | AAGCCCTTGAAGAGGAGTTGAGG | 1312 |
| hs132692346 | ACATGGGCAACAAATTAGATGGG | 1313 |
| hs132694165 | GCTGCTCAGGAACATCCTCATGG | 1314 |
| hs132684182 | GGATTCCCAAAAGTAAGTGAAGG | 1315 |
| hs132687526 | TGCCAGCATATGTAGTAAGTAGG | 1316 |
| hs132688757 | TATCCTTCACCTCTCTCAAGAGG | 1317 |
| hs132684883 | GCTGGGAATAGAAGATGTCAGGG | 1318 |
| hs132687897 | AGACTATTAAGAAGAACTCAAGG | 1319 |
| hs132685321 | ATGTAAACTAATAAAAACGTTGG | 1320 |
| hs132684322 | GTTGATATGCTAAATAAAAGGGG | 1321 |
| hs132687254 | AATTTATTCAGTATGTCACCAGG | 1322 |
| hs132688008 | TAGACTAAATTCAAAGACTGAGG | 1323 |
| hs132683891 | ATCATGACCTTACAAGAGGAAGG | 1324 |
| hs132684178 | TGATTTTTCATTGAACTCAGAGG | 1325 |
| hs132683964 | GTAGACATCTCACTGCTCATGGG | 1326 |
| hs132683585 | GAAAGAAGTGGCGTTTGCAGTGG | 1327 |
| hs132683924 | ATACAAAACAAAACATGAGAGGG | 1328 |
| hs132685426 | GTCTATTCATTCAGGCTAAGGGG | 1329 |
| hs132684351 | TGTATTTATAAACAACCCTAAGG | 1330 |
| hs132693233 | TGGGGAGAGGAATTGGTAAGGGG | 1331 |
| hs132693474 | TCTTGTACATCTTAACCTCAAGG | 1332 |
| hs132688930 | TAGCCACCTTCTGTATCAGAAGG | 1333 |
| hs132692993 | TTCCGATGATCCTTTAACTGTGG | 1334 |
| hs132690646 | ATGAAGAGACAAGCTTTCCAGGG | 1335 |
| hs132690259 | GCATTGAGCACACTCTACAAGGG | 1336 |
| hs132689772 | CACAGGAGCTCTAAGTCCAGTGG | 1337 |
| hs132694199 | GGAAAACAAGGTAAAGTGAGTGG | 1338 |
| hs132690050 | GGTGACCACAGTTTAAAGAAGGG | 1339 |
| hs132690729 | ACGGTGTTATCTTTAAAACCTGG | 1340 |
| hs132685909 | GACAAGCAGAACAATTTCAAGGG | 1341 |
| hs132684783 | CTAAGGGAACATCTTGCTGTAGG | 1342 |
| hs132691951 | TGGATCAGAATTGAAACTGAGGG | 1343 |
| hs132690190 | AAGATGTGAGCTATAAAAGAGGG | 1344 |
| hs132694137 | GCCTGAAATTGGTAAGAGGACGG | 1345 |
| hs132685661 | CAAGTTTCCTCCCTAGAGGAGGG | 1346 |
| hs132694062 | TATGTTGCAATTTGAAGACAGGG | 1347 |
| hs132686612 | AATTCCAAGGGACTTTTGAGGGG | 1348 |
| hs132683829 | GATACAGTGAATTCACAGCTTGG | 1349 |
| hs132685242 | CTGTTCAGTAAGTGTCTGCAGGG | 1350 |
| hs132693528 | AAGTCAACAACATACATGGATGG | 1351 |
| hs132687657 | TCTTGTGCGTGCTAAAGCATAGG | 1352 |
| hs132690119 | CAAAACAAAAGCCAAGAAGAGGG | 1353 |
| hs132688191 | TGCCCAGTGGTGGGTCAGAGGGG | 1354 |
| hs132690494 | CCAAAATTGATAGAAAACATTGG | 1355 |
| hs132690226 | TAATATGCTGTAATATATGAGGG | 1356 |
| hs132691254 | GATAATATCAAAGACTCTTGGGG | 1357 |
| hs132688099 | GTCACTACTAGAGTATGCACTGG | 1358 |
| hs132692564 | GCCATAGGCCATCTCCATAGTGG | 1359 |
| hs132693079 | CACCTCTTCAAAATAGTGTGTGG | 1360 |
| hs132683546 | ACACATATGTATATATCAGAAGG | 1361 |
| hs132692847 | TTTCTATAATCTATTGCCAAGGG | 1362 |
| hs132685169 | ATAAGAACATACATAGCCAAAGG | 1363 |
| hs132685305 | GCTTTGTGAAACAATTGGCAAGG | 1364 |
| hs132690962 | ACTCCATCAGAGAGTTCTGTAGG | 1365 |
| hs132687560 | AGGATAGCTTACTTATACAGGGG | 1366 |
| hs132690532 | CTTTGTTACATTTAGAACCACGG | 1367 |
| hs132688246 | TTTTCCCACTTCATAGACACGGG | 1368 |
| hs132693371 | ATGAAGTTCTACATTGAACTGGG | 1369 |
| hs132683473 | TTTAAATGATATTTCCTTGGGGG | 1370 |
| hs132687076 | CAGAAGTTACTGACTTGAGGAGG | 1371 |
| hs132683755 | TCTGTACTAAGATCGGTGACAGG | 1372 |
| hs132690683 | TAATACAAAGAAATTGCAAGTGG | 1373 |
| hs132685694 | TGAGAGATGAGCAAATAGCCTGG | 1374 |
| hs132690953 | GGGCTGAAGCCCATGCTGAGGGG | 1375 |
| hs132686673 | TGAACAGGAGGTGGATCGAAAGG | 1376 |
| hs132694103 | TTCAGGAAAATATAAGTCAGAGG | 1377 |
| hs132688577 | TTTAACATCAGAAAGAACGTTGG | 1378 |
| hs132688277 | ATAGAGAATTTTTAACACCTAGG | 1379 |
| hs132683580 | GTGGAGGCAAAGGAACAGACAGG | 1380 |
| hs132690431 | TCAACACAGTAACTGATGCCAGG | 1381 |
| hs132685374 | AGAACTCTGCTCAAGCTGAAAGG | 1382 |
| hs132684474 | CACATAATAATACTATCTAAGGG | 1383 |
| hs132689489 | ATAAACAAATACATATCTAAGGG | 1384 |
| hs132685648 | TGTTTGTTGCCAGGAAACACAGG | 1385 |
| hs132686409 | ATTTTGGTAAAATATGGTGAAGG | 1386 |
| hs132693460 | GGAGCTTGCAAAGGGACTGTAGG | 1387 |
| hs132689816 | TTATTGTTGGCTTTAAAAGGGGG | 1388 |
| hs132686476 | TAGAATAAATCTGAGCTAGGAGG | 1389 |
| hs132690503 | ATCTCATACACCTTTTGCTGGGG | 1390 |
| hs132685666 | CCAAAGCCAGTTTACTTCTGGGG | 1391 |
| hs132688364 | CTCTTACTCATAGTGATAAGGGG | 1392 |
| hs132688935 | ATTGTTCTGATAACCCAAGAGGG | 1393 |
| hs132687063 | AAGTTCCATGTCCTAGCTCGAGG | 1394 |
| hs132693813 | CCCAATTGTACTGTGCAGCATGG | 1395 |
| hs132690017 | TCATTAAATTGGAGATCGAAAGG | 1396 |
| hs132684265 | GGAGCTTCCACTAGGCCAGAGGG | 1397 |
| hs132689997 | TACAGTAGTCAGTTGAAATGGGG | 1398 |
| hs132685451 | TAGCTGGTAGATTAAAGCAAAGG | 1399 |
| hs132692962 | TTTCAAATAGAAGCTAAGAAGGG | 1400 |
| hs132685662 | ACAAGTTTCCTCCCTAGAGGAGG | 1401 |
| hs132688106 | GTATGTGCTAGCGTGGATGATGG | 1402 |
| hs132689154 | ATTTCTGGTTCATATAAAGGTGG | 1403 |
| hs132685979 | CCCTTTCACTGGTATTAGGGAGG | 1404 |
| hs132692447 | CTGAGGGTGGGAAAATGCCATGG | 1405 |
| hs132685112 | TTTATACTAACTATGGGTGGGGG | 1406 |
| hs132689054 | AACACTATCTGGCTGCCTGAGGG | 1407 |
| hs132689850 | GTCTATCACTCAAGGTACAAAGG | 1408 |
| hs132692308 | GTGTAGAAACCATTCTGCCAGGG | 1409 |
| hs132683353 | TTATTCACCCTTGTCAACAAAGG | 1410 |
| hs132685954 | TATTATTTTTTACATTCTGGAGG | 1411 |
| hs132692947 | CTATCCTAATAAGCAGGGGATGG | 1412 |
| hs132693977 | TCAGACCTCAGAGGTTACAAAGG | 1413 |
| hs132690669 | CAAGGAAGTTCAGAGATGTAGGG | 1414 |
| hs132684058 | GTACTCAATCCTTTGATGGAAGG | 1415 |
| hs132692428 | TGAGAGAGGCCAGACTTGCAGGG | 1416 |
| hs132687702 | GAAATGTGTACAAAGTTAGGAGG | 1417 |
| hs132693587 | ACTACATCCATAATCAGAGAAGG | 1418 |
| hs132689560 | TCACATTCCAAACCCAGCCTAGG | 1419 |
| hs132687538 | TGTTAATTGGAGGTTGACAAGGG | 1420 |
| hs132687015 | TTATATGCTTTGATCAGATGGGG | 1421 |
| hs132684780 | ATCTTGCTGTAGGCAGCCTGGGG | 1422 |
| hs132686192 | CCACTGTTCATTTTAACTGGGGG | 1423 |
| hs132694312 | AAAGTAACATTGGCCAAAGAGGG | 1424 |
| hs132687041 | TTTCATTTGGAGAGAACAGGAGG | 1425 |
| hs132683543 | TTGATTGATAAGTTATGCCAGGG | 1426 |
| hs132686130 | AGAGGAGATATTACCCCAGAAGG | 1427 |
| hs132693234 | TTGGGGAGAGGAATTGGTAAGGG | 1428 |
| hs132694202 | CTACTGAAATCATACACTAACGG | 1429 |
| hs132689394 | ATCATACTCTAATTCACAGAAGG | 1430 |
| hs132694154 | TTAGGAATATGGAGCAATGAGGG | 1431 |
| hs132692514 | ATTTAAACCACAGTTCTCAAAGG | 1432 |
| hs132684155 | TATTCCCTTCTCAACAGCCTAGG | 1433 |
| hs132694214 | TGCAGCCTAACAGATAAAGATGG | 1434 |
| hs132694039 | TGATGGACACATTAGTGAGTAGG | 1435 |
| hs132691773 | TTGAAGATGGGCGCTTCCAAAGG | 1436 |
| hs132689168 | TTTCACAGCAATACATTTTGGGG | 1437 |
| hs132692997 | TACCACAGTTAAAGGATCATCGG | 1438 |
| hs132693584 | AATGTATTAGTACTGTTGTGTGG | 1439 |
| hs132687576 | ACTTAACAATTATTATCTAGGGG | 1440 |
| hs132694037 | CTAATGTGTCCATCACATGCAGG | 1441 |
| hs132691666 | GACCATCTCTACATACAGAATGG | 1442 |
| hs132685513 | ACATCTAAGATATCCCAATACGG | 1443 |
| hs132684033 | TTACGCCTTTTAGAAGAGGAGGG | 1444 |
| hs132689696 | TATGCAAATTAATTTTTCAGTGG | 1445 |
| hs132688740 | TTTTTGAAAGAAGGACTAGTCGG | 1446 |
| hs132688221 | TCTGAAGACTGGCTAGACTAGGG | 1447 |
| hs132685293 | GGAGGTATTGTACTTTGCCAAGG | 1448 |
| hs132694418 | CCAAAATCTAACGCAGCACAAGG | 1449 |
| hs132690800 | GGAGAGAGAGACATGTACCTTGG | 1450 |
| hs132686743 | CTTTTGGTTAAAGAGTAATGAGG | 1451 |
| hs132688389 | TTGCAAATTGTTATAGACCAAGG | 1452 |
| hs132689132 | CCATATGTCATTATTAGCTGCGG | 1453 |
| hs132692745 | GTCTTGTGCAGTATTAGTAGTGG | 1454 |
| hs132684940 | GATATTGTAGGCATATTCGATGG | 1455 |
| hs132684358 | TGCCTACCAAGAGAAGAAGAGGG | 1456 |
| hs132693443 | TCTGCTCAGATCATTCAAAAGGG | 1457 |
| hs132685447 | ACTCAATTAACTATTCAGACTGG | 1458 |
| hs132693651 | CCACAGGGACAGAAGTAAGCGGG | 1459 |
| hs132688891 | AAGTGATTACACCTTGTACTGGG | 1460 |
| hs132686652 | ATACCTACTCCTCTTTGCTAAGG | 1461 |
| hs132691599 | TATCTGAGCTGTTACAAAAGTGG | 1462 |
| hs132691655 | GGGTTTCTGGAGACCGAGAAGGG | 1463 |
| hs132692585 | TCTACATCACGATAATATGTTGG | 1464 |
| hs132684836 | TAACTTCCATTGCCTATCAGTGG | 1465 |
| hs132687924 | GTCTTCTTTTAAAAATACAAAGG | 1466 |
| hs132683411 | GGAATTATCATCTCAGCCGCAGG | 1467 |
| hs132692220 | CACTTGCTCTTCCTTGCGGTAGG | 1468 |
| hs132684957 | ACCTGTTTACTACAAATCAAAGG | 1469 |
| hs132690772 | TTTTAAGGCATTCTGTAGTGAGG | 1470 |
| hs132691417 | TTTCTCGTGTTCAAGAGGCATGG | 1471 |
| hs132685624 | TATTTATTATAGGATGTGAAAGG | 1472 |
| hs132684812 | ACCCACATCAGTAGAGGGCATGG | 1473 |
| hs132684961 | GCTGACACAACTTTGTCCCCAGG | 1474 |
| hs132684588 | TTAAATTCAAGTCTCTCGAAAGG | 1475 |
| hs132689457 | CAGCAGTCCTCAGTAACTCAGGG | 1476 |
| hs132685396 | ATGTATGTGTGCGTGTACATAGG | 1477 |
| hs132693939 | ATACAGGTTTAAATATTGCATGG | 1478 |
| hs132686762 | TTCGGAACACAGTTGAGAGAGGG | 1479 |
| hs132694359 | AATAATCTTTATCACCACTAGGG | 1480 |
| hs132685977 | AGGGAGGAGATATCAAAAGATGG | 1481 |
| hs132691242 | CTGAGGACTAGATGGCAGAATGG | 1482 |
| hs132688158 | ATATGACTGAACTTAAAGGCTGG | 1483 |
| hs132684968 | TGTTCCTTCCAGAGGTGTGTGGG | 1484 |
| hs132689594 | TACACCTGCTTTTAGAACAGTGG | 1485 |
| hs132693507 | ATATAATTGAGAATTAGTGCAGG | 1486 |
| hs132692078 | ATAAGGACCAGAAGGTGAAGTGG | 1487 |
| hs132687759 | GAGTATTGTGTTACTAGTAGAGG | 1488 |
| hs132690847 | TTTTAATATCTAATTCAACATGG | 1489 |
| hs132685184 | ACTAATCTTAGGAAGCTGGCTGG | 1490 |
| hs132685159 | GGAATTTTACACCAGGTCGAAGG | 1491 |
| hs132692616 | GCCTGTCAGCGATACTACCTGGG | 1492 |
| hs132692323 | ATACCAGGAATCTCAGTCATGGG | 1493 |
| hs132687686 | TAGAGGGCTGATAATAACCTTGG | 1494 |
| hs132689604 | GCATCTTGAATTTTTAAAGGAGG | 1495 |
| hs132685421 | ATATTGACAAGGGAATGAAATGG | 1496 |
| hs132694182 | AATTCTAAGCATAAAGCTGAGGG | 1497 |
| hs132693375 | CCACAGATATATTCCTAGGAGGG | 1498 |
| hs132687676 | TTAACGTTATAATTTTTACAAGG | 1499 |
| hs132689007 | AAAAGCTTCGGCTTCATACGTGG | 1500 |
| hs132693831 | GGTATGATAGTTACCCTACATGG | 1501 |
| hs132690230 | AGAGTTACTCTCCCATTCGAAGG | 1502 |
| hs132683888 | TTTCCATCTCTCATTCAGATGGG | 1503 |
| hs132691223 | TGAATCCTGCTTTTTCAAGGTGG | 1504 |
| hs132692200 | ATCAGTTCCTCCACAACATGTGG | 1505 |
| hs132683662 | ACAAAAAAAAAAAAAAACAATGG | 1506 |
| hs132689243 | CGGATCATGATGAAAGCCACAGG | 1507 |
| hs132690289 | TCAAACTCAGTTAATGGAGGCGG | 1508 |
| hs132683376 | TTTGAAGTGATTTAATCTAGAGG | 1509 |
| hs132689977 | ATAAAAAGCAAAGAGAACATAGG | 1510 |
| hs132692306 | AATTAGTCTATAGGATAAGGTGG | 1511 |
| hs132693154 | AGACAGAGTCTGGGTCCCCATGG | 1512 |
| hs132692531 | GAAAACCATATACAAAGCATAGG | 1513 |
| hs132687910 | CATTCAAAAATACATGAACAGGG | 1514 |
| hs132692892 | GATAGGAGAATATAGACTGTAGG | 1515 |
| hs132694045 | TGAAATTGTATGGGTTTTAGGGG | 1516 |
| hs132684825 | TATATACATAATATAAGCCATGG | 1517 |
| hs132692798 | AAACAGGGTTTAATAGGTGAGGG | 1518 |
| hs132684172 | GAATAGGAAAAGAACAACTAGGG | 1519 |
| hs132686027 | TGCTTTTCAAAGGTGTGTGGAGG | 1520 |
| hs132693557 | TTAATAAAGTTTCTACAGAAGGG | 1521 |
| hs132688387 | TTACTGGAGAACCATGCAGTAGG | 1522 |
| hs132685074 | CTAAGATAAAATGTGACAGTAGG | 1523 |
| hs132688875 | CTCGAGAAGCCGCAAAACCAAGG | 1524 |
| hs132688404 | CTACATTGGCTAAGGAGCCAAGG | 1525 |
| hs132686707 | GAAAACTTATACATTTAAGTGGG | 1526 |
| hs132683515 | AATAGAATTAATATCTGCAGAGG | 1527 |
| hs132687551 | AATCTATTAAATGAAGTCCTGGG | 1528 |
| hs132684202 | TCTATGAATTTGCTATGCACCGG | 1529 |
| hs132684625 | CTGAATATTCTATCAAAGCAAGG | 1530 |
| hs132684648 | CCCTTGATCTCTGCTCAAAGAGG | 1531 |
| hs132690865 | TTCTGGAAAACTTAGCACGTTGG | 1532 |
| hs132690030 | TGTATTTTTATGTAGCGCGTGGG | 1533 |
| hs132686023 | TGCCGTGCTACTTAAGAGAAGGG | 1534 |
| hs132691871 | GAAATATGTCAAACAACTGGAGG | 1535 |
| hs132690523 | CTCACAGTGCAAGATACAAAGGG | 1536 |
| hs132692128 | TGAGGACGTGTGCAACGGGATGG | 1537 |
| hs132685348 | TAACACCTGCTGCTATGAAGAGG | 1538 |
| hs132688135 | TACACGAAACTGATGTCCAATGG | 1539 |
| hs132684423 | TTCTTCCCCACAGACCACATTGG | 1540 |
| hs132691625 | ATGTAACCATGCTTGACGCTAGG | 1541 |
| hs132690885 | ACTTGGGCTCTTAGAAAGTAGGG | 1542 |
| hs132687725 | AGTTTTCACAGAGAGCAAAAGGG | 1543 |
| hs132685541 | TAATTGTTATCAGTTGTACAAGG | 1544 |
| hs132692014 | GCCTATGGAACAATACATGTTGG | 1545 |
| hs132693839 | GCATTTCCCTAAACTGAATGAGG | 1546 |
| hs132687887 | TCCTGATTGTGGGAAATCAGAGG | 1547 |
| hs132684874 | AAATGAATAATCTTTTGCTAGGG | 1548 |
| hs132684157 | TCATCCTAGGCTGTTGAGAAGGG | 1549 |
| hs132685914 | TTTATATTAATAATCAAGAGTGG | 1550 |
| hs132687193 | TACTCAAGCACCTTTGTGCAAGG | 1551 |
| hs132687619 | TGTTCTTGCTATAAAATGTGTGG | 1552 |
| hs132689892 | TACTTCACTTGAACTTAGCAGGG | 1553 |
| hs132684746 | GTGCAAAATATGTTTGGCAGGGG | 1554 |
| hs132692561 | GTAGCATACCACTATGGAGATGG | 1555 |
| hs132689882 | AACTTACCACATAATCCGACTGG | 1556 |
| hs132687888 | CCTGATTGTGGGAAATCAGAGGG | 1557 |
| hs132689352 | AGTCAATGCTTATATGGGAAAGG | 1558 |
| hs132691804 | GCTTTAATAAGAGAAACCTCTGG | 1559 |
| hs132683893 | CTCTATATCTGCAAACTCAAGGG | 1560 |
| hs132688355 | ATTTGTATTAAGCGAAGCCATGG | 1561 |
| hs132691923 | ATCTTATGACACACCACCCTAGG | 1562 |
| hs132692077 | TAAGGACCAGAAGGTGAAGTGGG | 1563 |
| hs132689115 | GTTAATTTATTTCAGGAACATGG | 1564 |
| hs132690760 | ATGAAAACTTAGCCTTGCCATGG | 1565 |
| hs132687142 | TGTATGCTTACACTGTGTGTGGG | 1566 |
| hs132693418 | AAAATTGTACCACCAGTACAAGG | 1567 |
| hs132686901 | AAGTTTTTAATCAAAGAAGACGG | 1568 |
| hs132688421 | TCTAGCACAACAGGGTCCACAGG | 1569 |
| hs132691938 | GGCAGGTCCTAGTTTACCCTAGG | 1570 |
| hs132690375 | CCACAAGTCAAAGATGGGTGGGG | 1571 |
| hs132688888 | GATGAACAATCCAGATATAGGGG | 1572 |
| hs132691921 | CTAGAATGACTCTCAGCCTAGGG | 1573 |
| hs132692118 | ACTTAACTCTCTGTTGTGAGGGG | 1574 |
| hs132690322 | AGCAAATGACATGGAGTACAGGG | 1575 |
| hs132692609 | ATAGCTCATTAGCTCAATGAAGG | 1576 |
| hs132689208 | ACAAATAGCTGAGAGTTGGTGGG | 1577 |
| hs132693752 | AATGATGTCAACCAGTAGGATGG | 1578 |
| hs132687080 | AAGAAATTAGCTCTTGGCCAGGG | 1579 |
| hs132688832 | CACTGTGTTGGAAAATAAGAGGG | 1580 |
| hs132684261 | AAACGAAAGGAGCTTCCACTAGG | 1581 |
| hs132686993 | TTCTTCTCCTAGCATCACACAGG | 1582 |
| hs132685403 | GAGCTGTCAGTCATAGTGCTGGG | 1583 |
| hs132689179 | CATATCTTGTAAATATACTAGGG | 1584 |
| hs132692280 | CCTGAAAGCAAAATAAAATGTGG | 1585 |
| hs132693207 | AGTTGGGCAAAGACTGGGGTAGG | 1586 |
| hs132683739 | GAGAATACAAAAGAAATCCTGGG | 1587 |
| hs132693802 | CATACTGAAGGATTTGGGGTTGG | 1588 |
| hs132688258 | ACAAATACATAAATTTGGCAGGG | 1589 |
| hs132684152 | TTACCTGGCAGCCCACCTCATGG | 1590 |
| hs132687166 | ATCTTCACAAGCCCTTGAAGAGG | 1591 |
| hs132688336 | CTGAGGGATTGGAAGAGTCAGGG | 1592 |
| hs132687808 | TTTCAAATATCATTACTCCCTGG | 1593 |
| hs132689095 | TGTTGTATCTCTGTTCGTTGTGG | 1594 |
| hs132693814 | TCATCACTAATAGAAAATGATGG | 1595 |
| hs132692398 | ACACTACAATCATATATATGTGG | 1596 |
| hs132686523 | AGATCCCCCCATTACACTTGTGG | 1597 |
| hs132687466 | AGAAAGAAGTAGCAAATGCAAGG | 1598 |
| hs132686473 | TTCAAAATAATGAGCACTAGAGG | 1599 |
| hs132693798 | GCAGAGTACTACTGAAGTTGAGG | 1600 |
| hs132693192 | AGTATAAATCAACTGCGCATTGG | 1601 |
| hs132691934 | AGATTACACATGTTGTTAAGGGG | 1602 |
| hs132685097 | GTTCTTACTTCGTAGAATGGTGG | 1603 |
| hs132691493 | TTAATAGAACTAGAGAATCAGGG | 1604 |
| hs132692880 | TCAGACAGTGATGTTTCATGTGG | 1605 |
| hs132683889 | CTTCCCATCTGAATGAGAGATGG | 1606 |
| hs132687430 | GGGAGAAATGAGTAAAAATGTGG | 1607 |
| hs132687525 | AATTTCTATGAATTTCAAGTGGG | 1608 |
| hs132687115 | ACAGGGACTCTCTTCTAGTAGGG | 1609 |
| hs132693200 | GCAAGGACATGTAGGAAGGAAGG | 1610 |
| hs132688312 | ACAGTGAGAGTTGGATAGAGAGG | 1611 |
| hs132685267 | TCATATGTTCACTAGTACTGAGG | 1612 |
| hs132683315 | AAGGAATAATCATTTCTCCATGG | 1613 |
| hs132692223 | AGGATTTGTCAATATTCCAAGGG | 1614 |
| hs132694226 | ACATACTTTTCAGATAACATTGG | 1615 |
| hs132684871 | CAGGAAAAGGGGTTCAAGAAGGG | 1616 |
| hs132693866 | TTCAGCCAAAAGTAAAGAAGAGG | 1617 |
| hs132688304 | GGCACAGTAAGCCTTTGGATGGG | 1618 |
| hs132684985 | TTTTAAGTAAAAGGGACACCTGG | 1619 |
| hs132686637 | GGATTTAGTGTCAGATATGGAGG | 1620 |
| hs132691701 | CTATGGCACCTTCAGTGATAGGG | 1621 |
| hs132686562 | ATCCTGGCAGAAAACTATGCTGG | 1622 |
| hs132690520 | TATTGTACCTCACAGCATAGAGG | 1623 |
| hs132694006 | TTTTCCACATCACTCACACTAGG | 1624 |
| hs132684052 | AGGTAGAAGCCACTGAGAGAGGG | 1625 |
| hs132690184 | ATCACTCGTTCAAAATACTTGGG | 1626 |
| hs132685179 | AGGTAGCTAAGTATTAGGAAGGG | 1627 |
| hs132686083 | ACTATAACCTATGGATAGGATGG | 1628 |
| hs132691907 | ATATTGGAACTTACTCTGCTAGG | 1629 |
| hs132693974 | GGTTACAAAGGAGGGCTGAAAGG | 1630 |
| hs132689105 | CCTAGTATCTTGATAACCCCAGG | 1631 |
| hs132692802 | CTTTTATATGTTTAGCCTAGAGG | 1632 |
| hs132691606 | TCATGCATATGATTACAGAAAGG | 1633 |
| hs132684387 | ATAGTGCCCAACCAGAGCAAAGG | 1634 |
| hs132687103 | CTGTGACGACGATCATTGGTGGG | 1635 |
| hs132690982 | AAGAGAATTACGTTAAGGGATGG | 1636 |
| hs132684582 | AATACTGTAGGTTAAGAAAGTGG | 1637 |
| hs132694196 | AAACCCACGAAGAGAGAAGAGGG | 1638 |
| hs132691555 | TGGACAAGATTGAGGAACAAAGG | 1639 |
| hs132691151 | AGGATGGTATCAGGTTAGCTTGG | 1640 |
| hs132691572 | TCATATTTAACAAATCTAAGTGG | 1641 |
| hs132686074 | TAACATGAAAAAATTGGTAGGGG | 1642 |
| hs132688053 | TTGTTATTAGCCACAAATCAGGG | 1643 |
| hs132687935 | TAGAACAAAGTGGCAGGCATGGG | 1644 |
| hs132692094 | AGACCACCCTAAAAATCACCAGG | 1645 |
| hs132685041 | CCTTAAAATGTTAAGTACTGAGG | 1646 |
| hs132686645 | GATCAGTCAGAGTCCAGGCTTGG | 1647 |
| hs132683368 | GCACATGTAGGAAATATAAGTGG | 1648 |
| hs132691113 | TAGTATCATAATCTTTACCATGG | 1649 |
| hs132685485 | TTATCTGTATACAAATTCACCGG | 1650 |
| hs132692028 | TTTCTATCACAGATTTACATGGG | 1651 |
| hs132687457 | AAGCACTGAGAATTAGGGATAGG | 1652 |
| hs132690172 | ATCCAGACTACTAAGAACATTGG | 1653 |
| hs132691609 | AGTATTGACAGTCTGACCATGGG | 1654 |
| hs132694072 | TGAGCAAACGTTTATAGAGGAGG | 1655 |
| hs132688475 | CAAGGGCATCAAGTAAGAAGAGG | 1656 |
| hs132692409 | GAAAGAAACTTAACTAGCAGTGG | 1657 |
| hs132692728 | AGATGTATTTTTAAGCTAGGAGG | 1658 |
| hs132693182 | GGATGAGTAACTTTTGAAGGAGG | 1659 |
| hs132691642 | ACTCCGTTACACGTTTACAAAGG | 1660 |
| hs132685175 | GGTTAAACGGAAGCAAAAGTAGG | 1661 |
| hs132689488 | TTATTGTTCTAGCAGCAAGAAGG | 1662 |
| hs132694331 | TCTGTTTTGAAAAATAGTCAAGG | 1663 |
| hs132689800 | TTACAATATATTGAGTACACTGG | 1664 |
| hs132691632 | ACACCTGTCATCTCAAAAGAAGG | 1665 |
| hs132688769 | GCTCATTTAGGTAGTTTGAGTGG | 1666 |
| hs132690903 | TTGAACTCCTAATTAGAGGATGG | 1667 |
| hs132687664 | CCATATTCTTAAGACAACTTGGG | 1668 |
| hs132684834 | CCACATCCACTGATAGGCAATGG | 1669 |
| hs132689460 | AACAGACTCTTAAGCAGCAGTGG | 1670 |
| hs132687915 | TGAAACATATAAGTTATGACAGG | 1671 |
| hs132687627 | AGTGTAATCTAGCACAGCAATGG | 1672 |
| hs132690894 | CTTTTCATGACTCTATCTGGTGG | 1673 |
| hs132691985 | GTGACTAGAAAATCTGAACTAGG | 1674 |
| hs132693727 | AGAGAATAAAAGGCCCAGAATGG | 1675 |
| hs132692670 | ACAGTGCCTAAAGTCGAAAAGGG | 1676 |
| hs132691827 | AATGCGAAGCCACTACCCATGGG | 1677 |
| hs132692099 | TCACCAGGGCTGAGAATAGGAGG | 1678 |
| hs132691581 | TTACATTTCTAAATAAATGTGGG | 1679 |
| hs132690220 | ATGAAATACTTTAACACCCAGGG | 1680 |
| hs132687334 | ATATACCGCCTATAGTTGATGGG | 1681 |
| hs132686616 | AGATAGCTAGGCTAATTCCAAGG | 1682 |
| hs132693695 | CATTAGCTGCTTTCTAGCTGAGG | 1683 |
| hs132693946 | CAACACATACAATAAATGTAGGG | 1684 |
| hs132690416 | TCAAACTTTATTGCTCCAGTAGG | 1685 |
| hs132689250 | ATTAGTTCCTTTACTCAACAAGG | 1686 |
| hs132691880 | ATATAACACAGAAAGTAAAAGGG | 1687 |
| hs132688724 | GTGAGTAACTAGAAATGCATGGG | 1688 |
| hs132683830 | TTCACTGTATCTATGCAGAAAGG | 1689 |
| hs132693221 | AATTAAACTCCAATATATTGAGG | 1690 |
| hs132694124 | CATAATCACTGAGCTTCTCAGGG | 1691 |
| hs132688226 | AGATGAATGTGATTTAAATGGGG | 1692 |
| hs132684845 | GTTACATTTTCTATGACTAGAGG | 1693 |
| hs132687305 | AGAATGAAAATCTATCTGGTTGG | 1694 |
| hs132688820 | GGCTTAATAGAAGATCAGTTGGG | 1695 |
| hs132688892 | GATTACACCTTGTACTGGGATGG | 1696 |
| hs132692529 | ATTGTGTTCAAAATTTACACTGG | 1697 |
| hs132691942 | CCAACCAGCAAAATAACCCTAGG | 1698 |
| hs132690731 | TTTATAGTAGGCAGTGCATACGG | 1699 |
| hs132684046 | GAACAGGCGGGTCCCATGGGTGG | 1700 |
| hs132690925 | ATCGACTCTTGGAATGGCAGCGG | 1701 |
| hs132693170 | TGGTTCCAGTGAGATACAGCAGG | 1702 |
| hs132684424 | GGAAGAAGATCTTTGCATGCAGG | 1703 |
| hs132688386 | TACTGGAGAACCATGCAGTAGGG | 1704 |
| hs132688948 | GCCTTGTAGTTATCCCACATGGG | 1705 |
| hs132691092 | ATATGTGCTCAAGTGAGTACAGG | 1706 |
| hs132693315 | TTTGATGGAAGTATGAGTAAGGG | 1707 |
| hs132690547 | ACAATAACTGGAAAAGTGAAAGG | 1708 |
| hs132687368 | GTGTTTTAAGAAGATATATGTGG | 1709 |
| hs132693505 | TATAATTGAGAATTAGTGCAGGG | 1710 |
| hs132686420 | GAAATTGCTAATACAATGGACGG | 1711 |
| hs132691911 | CAAGGATAGGACTTTATCCATGG | 1712 |
| hs132690305 | GGGTATAAAGTTTCCTAAGAGGG | 1713 |
| hs132691477 | TTATGAGTCAGTTTGAAATGAGG | 1714 |
| hs132692081 | TGAAAAAAATAAGGACCAGAAGG | 1715 |
| hs132687322 | TGAGAAAAGCTTAAAGCTAACGG | 1716 |
| hs132689449 | GAGGGCAAAAGAACTGTAGACGG | 1717 |
| hs132686813 | GCTTTTGATGTATTCTAATGGGG | 1718 |
| hs132686208 | ATATGAGGAAATAGATCACCAGG | 1719 |
| hs132686176 | ACATAACAGACTCCTTTCACAGG | 1720 |
| hs132691821 | ACAGACTATTTTCCCCCCATGGG | 1721 |
| hs132685258 | CCAGGCAAAGAAAGAAGAGATGG | 1722 |
| hs132684195 | TTAAGACTCATTCAGGACTCAGG | 1723 |
| hs132692332 | TTTTCCAAACGTTTATAACAAGG | 1724 |
| hs132688051 | GTAGAGCTGTGTGAATCCATGGG | 1725 |
| hs132693465 | GTAAACATCATCAAGGCAGATGG | 1726 |
| hs132693381 | GTAGTTAAGGTACTAAAGTAGGG | 1727 |
| hs132689229 | GCAGGAAGTTGGGAGGCTGGTGG | 1728 |
| hs132690231 | GAGTTACTCTCCCATTCGAAGGG | 1729 |
| hs132689790 | AAAGGAATCCCCACAAATTGGGG | 1730 |
| hs132688695 | AAAAATCATATACATAGAAGTGG | 1731 |
| hs132685425 | TCTATTCATTCAGGCTAAGGGGG | 1732 |
| hs132689839 | ACAAGCGCTAGAAGTTATGCTGG | 1733 |
| hs132688606 | ATGATCTCAGAGTACTACATTGG | 1734 |
| hs132683405 | TACTGGTAGCAGAAAGAATGAGG | 1735 |
| hs132691685 | TCACCGTGATTACTAATGGATGG | 1736 |
| hs132685725 | TTTTAAACGTCCTTTTGTAGAGG | 1737 |
| hs132692972 | AAGGCCTAGTGAGGAAAGATGGG | 1738 |
| hs132693749 | ACTGGTTGACATCATTACGTTGG | 1739 |
| hs132686039 | GTACACGTTGTCTTTATGAAAGG | 1740 |
| hs132686655 | CATCTGATGCCTTAGCAAAGAGG | 1741 |
| hs132690733 | GCACGAATGTCAGTGTCGTATGG | 1742 |
| hs132688704 | TGCATTTTATTAAATTAGAGTGG | 1743 |
| hs132689814 | ACAATAAGTAAGCTTGTGAAGGG | 1744 |
| hs132688812 | TGGGGTACGATACAGTGTAAAGG | 1745 |
| hs132691955 | CTCCTTCGTTATGAATCTGGAGG | 1746 |
| hs132690243 | ATTATTTTACCAAGTTTGGAGGG | 1747 |
| hs132687748 | AAGTTGAAATACGTTCACTAAGG | 1748 |
| hs132694115 | GTATTTCGTAAGAGGATGCTGGG | 1749 |
| hs132686875 | CACACGTTTTTAATTTAATGCGG | 1750 |
| hs132692850 | ACCAGAAAAGAGTTAAAGCATGG | 1751 |
| hs132688325 | TGGAGATGAAGCCCTACACTAGG | 1752 |
| hs132686983 | GACGACACAGTTATTTCTAGAGG | 1753 |
| hs132689340 | GGAGAGCCTACATGTAGCCTAGG | 1754 |
| hs132686214 | AATCACATTTGATGTTGTAAGGG | 1755 |
| hs132688308 | TAATTACCTTCCCCATCCAAAGG | 1756 |
| hs132693917 | TTATCCCTAAGACCTATGTATGG | 1757 |
| hs132686682 | CACTTAAGATGTTAGAGGGAGGG | 1758 |
| hs132688864 | GCATCTTCTGTGACTATGCAGGG | 1759 |
| hs132688292 | TGATAAAAATTATTGAAAAGGGG | 1760 |
| hs132693251 | TGTTCCTTAGTATTGAGGGAAGG | 1761 |
| hs132689505 | CCATACCTAACCTCCATTGGCGG | 1762 |
| hs132687143 | TGTGTGGGTTTTTATGCCTGTGG | 1763 |
| hs132690301 | CTGACAACACAGAACGAACCAGG | 1764 |
| hs132684191 | TGTGTGTTTCAGAGTTGGGATGG | 1765 |
| hs132683322 | ATACGTGCATGCTTTAAGGAAGG | 1766 |
| hs132692553 | GGCTGACTGGACCAAGATTGAGG | 1767 |
| hs132689721 | TATTATAAAAGCAGGTCAGAGGG | 1768 |
| hs132686789 | TCCTACAGACAATTTTGCCAAGG | 1769 |
| hs132693736 | TATATTTGAGGAAATAATGAGGG | 1770 |
| hs132687745 | GGCTGACACAATCATAAGCATGG | 1771 |
| hs132691863 | TTTCATCTAGACTGAACTCATGG | 1772 |
| hs132685279 | CCTAGTAGATTGTAAGGCCAAGG | 1773 |
| hs132693050 | AGTATTTTGCATGAAGCCAAAGG | 1774 |
| hs132688723 | TGTGAGTAACTAGAAATGCATGG | 1775 |
| hs132683614 | GTTAGATACAATGCAGGCCGGGG | 1776 |
| hs132689367 | ATTAGCAGTAGATGTATCTAGGG | 1777 |
| hs132689910 | CTAGTGTATGAGATTAGATGAGG | 1778 |
| hs132690724 | CTTTTCTTTTAATATACCCAAGG | 1779 |
| hs132688653 | ATATTAAAGATTGAAGTCAATGG | 1780 |
| hs132686654 | ATGCCTTAGCAAAGAGGAGTAGG | 1781 |
| hs132685688 | GTATGTGCATTAGAGAGCTATGG | 1782 |
| hs132691273 | TCCTTTAAGTAAGACAGCCAGGG | 1783 |
| hs132692376 | GCTAGAGAAGGTCAGAAGAAGGG | 1784 |
| hs132684153 | ATGAAGCCTTCCAATTTACCTGG | 1785 |
| hs132693301 | GCATAAAATCTACCAGTAGGTGG | 1786 |
| hs132683541 | AAGATCTAGGTCGAATGTGAGGG | 1787 |
| hs132693766 | GATATAACCACAATCAGAAGTGG | 1788 |
| hs132691030 | GGAAACAACTTGAAAGAGGAAGG | 1789 |
| hs132691167 | AATGGAAGAATTTATCTGGTAGG | 1790 |
| hs132685095 | TTACTTCGTAGAATGGTGGTTGG | 1791 |
| hs132685727 | TACTTCATTTTTCTCCCGAGAGG | 1792 |
| hs132690613 | CATCGTTATGTGAATGCCATGGG | 1793 |
| hs132693623 | TATTACAATGTAATTAGCCATGG | 1794 |
| hs132694129 | TTTCTTTTTCCTGATTCCAAGGG | 1795 |
| hs132693966 | TCTCTTTTTAATTTCCTAGGAGG | 1796 |
| hs132692042 | CTATAATTTAATAAATCCAAGGG | 1797 |
| hs132690238 | AGGGAGCTTGAAATTTTGCAAGG | 1798 |
| hs132694029 | TGAATGGGTCAGCAGCCAAAGGG | 1799 |
| hs132684259 | AGATTTAGATTAACTGGGGTAGG | 1800 |
| hs132686112 | TGAATACCAAGAGGATGGAAAGG | 1801 |
| hs132684258 | ATCTAAATCTGCAAAACGAAAGG | 1802 |
| hs132693238 | GAAAACACTCAATTGGGGAGAGG | 1803 |
| hs132694371 | AAGCAAATGGAGCATGAGCATGG | 1804 |
| hs132685002 | AATTTGCCCTTATGAAAGGAAGG | 1805 |
| hs132684586 | GATTTATAAAAGGCAAACATGGG | 1806 |
| hs132693544 | ATTAAGCACTATGTCTGCATAGG | 1807 |
| hs132691182 | TTAGTTTAAAACAATACTGAAGG | 1808 |
| hs132686749 | ATTAAAAAGAACTTTCCCACAGG | 1809 |
| hs132686817 | TAGTTTCAGTTTTCAAACTGTGG | 1810 |
| hs132692431 | CTCTCTCATGGACAGTCAGAGGG | 1811 |
| hs132688678 | AGAGCTTCTTATTGAGAAAGGGG | 1812 |
| hs132693248 | TATGCTCACCCTTTACAGAAAGG | 1813 |
| hs132683352 | AGTATTTCCTTTGTTGACAAGGG | 1814 |
| hs132692968 | CTCCGTGCAGATTCTAAGGAAGG | 1815 |
| hs132686306 | GAAACCAGCAGGGACCTGAATGG | 1816 |
| hs132688491 | GCTTTCTGAACTGATTGGAAAGG | 1817 |
| hs132691348 | AGTATCCCTTGATACCTGAATGG | 1818 |
| hs132684271 | GGATGGGAAAACAATCAGAATGG | 1819 |
| hs132688461 | ATGAAATGTTTCTCAGCTAAGGG | 1820 |
| hs132693648 | GATGTTGGAGCAATAGCCACAGG | 1821 |
| hs132689987 | GGGATAATCACTTTCACTAACGG | 1822 |
| hs132689461 | ACAGACTCTTAAGCAGCAGTGGG | 1823 |
| hs132688212 | ATATCTGAGCAACAGATGCTAGG | 1824 |
| hs132686494 | TATCTCAAGAGCAAACACTTAGG | 1825 |
| hs132689067 | TTGCATCAGTTTCTAAACGAGGG | 1826 |
| hs132692721 | TAGTTTATATTACTTGAGGGTGG | 1827 |
| hs132684513 | TATTCACTGCCAATAACTTGGGG | 1828 |
| hs132687996 | GATGAAAAACAACTGCAGAAAGG | 1829 |
| hs132686542 | CAAAAGTTCATAGAAAAAAGAGG | 1830 |
| hs132693431 | CAACCCACTTGCATAGTGTAGGG | 1831 |
| hs132691500 | TCTAAGTGACAAAATTCTGATGG | 1832 |
| hs132683944 | TTGTCTTCTATGGCAAACCATGG | 1833 |
| hs132689175 | GATAGATTTACAGAAAGAGAAGG | 1834 |
| hs132691481 | AAGTATAGTTTCTCAATAGGAGG | 1835 |
| hs132686500 | CATATTCATTATGAATGCTGAGG | 1836 |
| hs132688784 | ATATTGAGATACTTGCCATAAGG | 1837 |
| hs132688137 | TGACAGCAGTCAGTTCATTGAGG | 1838 |
| hs132686755 | TTGAAGAGATAAAAACAAGCGGG | 1839 |
| hs132694067 | TGCTGTCAATGTACACCTGAAGG | 1840 |
| hs132686867 | GAGAGAGAAGACTTGCAAAAGGG | 1841 |
| hs132693542 | TTAACTAACAGACTAATGGAAGG | 1842 |
| hs132686849 | AGCATAATTTAATAAATAGACGG | 1843 |
| hs132693109 | TGGGTAATAGGAATATGGGATGG | 1844 |
| hs132689415 | AGTGGGGCAAAAAATGAGGTAGG | 1845 |
| hs132688584 | GTATGGTACTGGGATTGTAGTGG | 1846 |
| hs132692148 | TTTATAACCCTGATATTCCAAGG | 1847 |
| hs132693077 | ACAGTTCCTAAGGCCTTACCTGG | 1848 |
| hs132690279 | TTTTGATTGGATTCAGAGAGAGG | 1849 |
| hs132694220 | CTACTCCACTCGCATCATCCAGG | 1850 |
| hs132683941 | TTCATATTTCAATATCCCCATGG | 1851 |
| hs132687312 | GGAAGAACTTGAGCCAACTTGGG | 1852 |
| hs132684323 | TTGATATGCTAAATAAAAGGGGG | 1853 |
| hs132684400 | ACATGTGTCGGTGCTTGAGAAGG | 1854 |
| hs132684102 | CAGAGATCCTACAAGAATCCCGG | 1855 |
| hs132689837 | CTAGAAGTTATGCTGGTCCACGG | 1856 |
| hs132684200 | CATTCGTGTGTGCTGTGTTGTGG | 1857 |
| hs132690643 | AGTTAGTGCACAAGAGGCGTTGG | 1858 |
| hs132686831 | TCGCAGAGAATAAGAATCAAAGG | 1859 |
| hs132691347 | ATGTTCCATTCAGGTATCAAGGG | 1860 |
| hs132688670 | TGGGGAAAAAACAGACCAGATGG | 1861 |
| hs132689205 | TTAGTTGACAACACTGCTGAAGG | 1862 |
| hs132686016 | GCTTAGCATAACCATAACCTAGG | 1863 |
| hs132693033 | AGATAGGAAAAGGTTAGTTGAGG | 1864 |
| hs132683968 | GTGAGATGTCTACCAATGCCGGG | 1865 |
| hs132693392 | GGCCACCTGCTCTTATGCGTAGG | 1866 |
| hs132692792 | AGAAAGAAAATATACAAAGGAGG | 1867 |
| hs132691756 | CAGATGATCCTACAGTTATGAGG | 1868 |
| hs132683804 | ATATTACTCTTCAGCCACCTAGG | 1869 |
| hs132685699 | ATATCTAAATGTGAAGTTGGCGG | 1870 |
| hs132691464 | GAGAGGATCCAATGCCACATTGG | 1871 |
| hs132691081 | AAGCACTATGGTGTGCCACTTGG | 1872 |
| hs132691848 | GAATCTAGGCAGCTATTCCAGGG | 1873 |
| hs132693483 | AGGAAAGGGTAGTTTTCAAGAGG | 1874 |
| hs132686377 | TGACATCAAGGAATATTAAGTGG | 1875 |
| hs132684429 | CTTGACCTAGAATCAGAAGTTGG | 1876 |
| hs132689063 | GTTATCTAAAATAATCAGCATGG | 1877 |
| hs132693797 | GCCAGCACACAGGTGCCTTGAGG | 1878 |
| hs132694326 | TCATAATTCATATATACAGCAGG | 1879 |
| hs132688378 | GCTCCCCCCACCCTACTGCATGG | 1880 |
| hs132687314 | TCTGTTTGATGAAGGAGGCTGGG | 1881 |
| hs132683940 | TGCATCTAACATAATAAAGAGGG | 1882 |
| hs132693305 | CCAATGAGTGACCCACCTACTGG | 1883 |
| hs132691670 | ATGATGCTAAAAAAAATTGGAGG | 1884 |
| hs132687976 | AAAAACTGAGCATTATGCAAGGG | 1885 |
| hs132689595 | TTCTAAAAGCAGGTGTAAGAGGG | 1886 |
| hs132689592 | TATCCCAGATAAGAGCCTTAGGG | 1887 |
| hs132694192 | TGATCAAATATGTTCTAGAAGGG | 1888 |
| hs132690972 | ATTCCTACAGAACTCTCTGATGG | 1889 |
| hs132685750 | TATATATACGTGGCTTATGAGGG | 1890 |
| hs132687173 | ACATTTTCGTTGGCAGTGAAGGG | 1891 |
| hs132690300 | TCGTTCTGTGTTGTCAGACAGGG | 1892 |
| hs132693889 | TCTGGATTTAAATTTCTAAGTGG | 1893 |
| hs132685264 | ATTTTCTTAGAATATGGCTAAGG | 1894 |
| hs132688326 | AGTGCTATCATCCTAGTGTAGGG | 1895 |
| hs132687120 | GAATGGTAAGTTTGTCTGCAGGG | 1896 |
| hs132692226 | CTCAGGGTAATGCTTAGAGGTGG | 1897 |
| hs132688691 | TCTTTTTTTCACCTACTCGTAGG | 1898 |
| hs132690087 | AAAATAACTAGGAAGCAAAAGGG | 1899 |
| hs132684797 | TAATGCCCATGTGAGAAAAGAGG | 1900 |
| hs132688025 | AATTGATAGCAAGATTTCAAGGG | 1901 |
| hs132691480 | TATAGTTTCTCAATAGGAGGTGG | 1902 |
| hs132686379 | ATATTAAGTGGCACATTGAAGGG | 1903 |
| hs132690573 | TATATTACTTTGGAAGTGCAGGG | 1904 |
| hs132690096 | GAAAAATCACAAAATCAGAATGG | 1905 |
| hs132688501 | TGGCAGGATACAGGAATACCAGG | 1906 |
| hs132684250 | ATTAGGCTCAACTTTTCAGTCGG | 1907 |
| hs132692130 | CTAGTTGAGGACGTGTGCAACGG | 1908 |
| hs132685543 | TATTGTAGCTCAAATGTTGGAGG | 1909 |
| hs132690526 | ATAAACCCTTGTTTAGGCCAAGG | 1910 |
| hs132690715 | ATAAATACACATTTATTCCACGG | 1911 |
| hs132690177 | CTACGCAAAGTCACCTTCAGAGG | 1912 |
| hs132685780 | GCATATGAGTTTGTGAGCGAAGG | 1913 |
| hs132685926 | TGATATCCAAAAATGAAGACAGG | 1914 |
| hs132686091 | CTCTTCCAGCAGCTTCCTAGTGG | 1915 |
| hs132686188 | ATTTAGCTCTGATAATTACAAGG | 1916 |
| hs132690224 | ATGTGCCTTCTTAAACCAGATGG | 1917 |
| hs132693838 | CTATTTCACACCGTAAAAGTCGG | 1918 |
| hs132691038 | AGTGTAGTCATCTTTGTGGTAGG | 1919 |
| hs132683761 | TAATAGATGTGAGTGTAGGTTGG | 1920 |
| hs132684800 | TGAGTTAGAAGGTAGAGGAGTGG | 1921 |
| hs132693640 | GAATTAAGCGTTAACAAAGAAGG | 1922 |
| hs132690246 | AGTTAAAATGGCAACACCATGGG | 1923 |
| hs132694108 | ATACTTTGAATCTAAGGGAAAGG | 1924 |
| hs132685060 | ATATGTTCTGTACATCAGCCGGG | 1925 |
| hs132684207 | CACTGTACATTGACTTCCCAGGG | 1926 |
| hs132688385 | TGGAGAACCATGCAGTAGGGTGG | 1927 |
| hs132689769 | CTAAGTCCAGTGGGTGTCTGAGG | 1928 |
| hs132688481 | TTAAAACAAGAAAGCTCTAGTGG | 1929 |
| hs132688766 | ATAAGTATGTTTGGCAGTGGTGG | 1930 |
| hs132691662 | TTTATGTTTAGAATATAGAAGGG | 1931 |
| hs132687456 | AGTGCTTTAGGCTATTTCCCAGG | 1932 |
| hs132687161 | AATCCTCAACTCCTCTTCAAGGG | 1933 |
| hs132683324 | ACGTGCATGCTTTAAGGAAGGGG | 1934 |
| hs132684894 | AAAGGGAAAATAATAAGCTAGGG | 1935 |
| hs132694089 | TTACTAATATCTATATAGAGTGG | 1936 |
| hs132691140 | GCCATACTTCTGTCAACCACTGG | 1937 |
| hs132690614 | ACATCGTTATGTGAATGCCATGG | 1938 |
| hs132683760 | CATTTAATAGATGTGAGTGTAGG | 1939 |
| hs132683482 | CACGTAGTGAAGAAATGCAATGG | 1940 |
| hs132688949 | GGCCTTGTAGTTATCCCACATGG | 1941 |
| hs132690278 | TTGGATTCAGAGAGAGGCAATGG | 1942 |
| hs132684918 | GAGTTGTGACTCGCTAAAGAAGG | 1943 |
| hs132687061 | AAGCTTCTATCTGATTGAAAGGG | 1944 |
| hs132691588 | TCATCCGTATTTTATAGGAAAGG | 1945 |
| hs132685958 | TATCACCTCAGAGATATGCTAGG | 1946 |
| hs132689740 | AATTTGGGCATACTTGTATGGGG | 1947 |
| hs132688252 | AACGCGTTTAGGAAAAATAGAGG | 1948 |
| hs132693806 | ATTTTCTGATGAAAATTCAGTGG | 1949 |
| hs132684875 | ATAATCTTTTGCTAGGGCTGAGG | 1950 |
| hs132685462 | TTTCAGTGAGGTAACCAGCCAGG | 1951 |
| hs132691917 | CATGTACCTTTCAAGGGAGTAGG | 1952 |
| hs132684667 | AGATTTCTAAATGGCTACACAGG | 1953 |
| hs132688735 | TTAAAGCCTAGAGCATAGACAGG | 1954 |
| hs132692612 | ACCCAGGTAGTATCGCTGACAGG | 1955 |
| hs132693989 | AATCCTCCTGAAAGTTTGCATGG | 1956 |
| hs132688976 | TCCTCAGATGTCTATGCGAAAGG | 1957 |
| hs132689337 | CAGTAGCTCAGAATGAGATAGGG | 1958 |
| hs132690115 | CCAAAAGCAGAGTGGAAGAAGGG | 1959 |
| hs132683312 | ATCATTTCTCCATGGTTGGGTGG | 1960 |
| hs132692486 | TTTAACATAAAAAGTAGAAGAGG | 1961 |
| hs132692730 | CTTTTTTAAGGATGAAGGAGGGG | 1962 |
| hs132690318 | CTTTTATTAAACTACACAAGTGG | 1963 |
| hs132687299 | CAAACTTGCTGTAACCCTATGGG | 1964 |
| hs132688348 | AAAATTCTCTAATAAACTGGAGG | 1965 |
| hs132690820 | ATTTCCCCGTTTTCATACTGAGG | 1966 |
| hs132689735 | TATTGCATGACTCAAATACATGG | 1967 |
| hs132685261 | TCCAATTACAATAATCAAAGAGG | 1968 |
| hs132684087 | TCGAGCCCCGCGAGAAGGAGGGG | 1969 |
| hs132683606 | GTATATGAAAAGGTGTTAAGTGG | 1970 |
| hs132690632 | CATATGCCTCACTAAGGTCCTGG | 1971 |
| hs132693879 | GAGAACTGTTTGTATGGCTAGGG | 1972 |
| hs132685605 | TCAGACAGACTATCATTCAAAGG | 1973 |
| hs132689572 | ACTCTCCTGAAGCATAAACTTGG | 1974 |
| hs132688047 | ATAATCTTACATTCTCCCCATGG | 1975 |
| hs132685527 | ATATTATAAATTAGGCCATAGGG | 1976 |
| hs132690803 | ATGTACCTCATTTGCTAACGTGG | 1977 |
| hs132683538 | TAGTGAGTGTCCTCATTCTAGGG | 1978 |
| hs132692929 | TCTGTGTTTCTGGATGAAATGGG | 1979 |
| hs132693984 | AGACGTACATAAACTACCAATGG | 1980 |
| hs132690822 | ACAATTGGCTCCTAGAAGGTTGG | 1981 |
| hs132684076 | TTCCGCCCCTCCTTCTCGCGGGG | 1982 |
| hs132691489 | TATGGACCAAGAATAATGAATGG | 1983 |
| hs132693792 | GCTATGCATTAGAAGGAAGTAGG | 1984 |
| hs132686375 | ATTTCAGCTGCTCAAATGGAAGG | 1985 |
| hs132685411 | TCTTATGTAAAGAACACAAAAGG | 1986 |
| hs132693052 | GGCTTCATGCAAAATACTGAAGG | 1987 |
| hs132685356 | CTCATAGGCAATGTATTCCAAGG | 1988 |
| hs132694170 | GAAATTCCTGTAAAACCCATAGG | 1989 |
| hs132693899 | AGTTGTTTCTCAGGCATCTGTGG | 1990 |
| hs132691102 | CCATCAACCCAGTATTATGTAGG | 1991 |
| hs132692776 | ATTTGTAATACAAATTACGATGG | 1992 |
| hs132689609 | CTCTGAGCAACATATTACAATGG | 1993 |
| hs132684949 | CAACACCCAGATTAAGGCATCGG | 1994 |
| hs132692759 | CAAAACCCCAAAGATTGTTACGG | 1995 |
| hs132692396 | ACATGTCATATGCTTTCCCATGG | 1996 |
| hs132689919 | ATACTAAACAATTGATACCATGG | 1997 |
| hs132689238 | TGTGGTCACCAAGTCACCTGTGG | 1998 |
| hs132683885 | TGAGTATTAATTAGGACACTTGG | 1999 |
| hs132685899 | CAAATCAAGCAACACTCACACGG | 2000 |
| hs132692472 | ACTCATCTGTAATTTCAAGTTGG | 2001 |
| hs132686505 | AACTCTATAGTGATAACAGCTGG | 2002 |
| hs132685864 | GTAACATGTTTTGATTGAAACGG | 2003 |
| hs132692735 | AAGTCATGTTTTTCAAACAACGG | 2004 |
| hs132688778 | CCTGCATTTAACGCATTAGGAGG | 2005 |
| hs132689199 | ATATGTATGTATATATTCATGGG | 2006 |
| hs132684069 | AGGCGGTAGCTATTTAGGCTTGG | 2007 |
| hs132692270 | CAGCATATGCTAATTGCGTATGG | 2008 |
| hs132689706 | TATATTATATAAGGCCTGGATGG | 2009 |
| hs132688964 | AGAAAATTAGTTCTTGCCTAGGG | 2010 |
| hs132691365 | TCTGGCTGACTAATTAGCAATGG | 2011 |
| hs132693653 | ACAGAAGTAAGCGGGTTAGAGGG | 2012 |
| hs132684331 | GGAAAGTCCTTTTTTTACAAGGG | 2013 |
| hs132691920 | GCTAGAATGACTCTCAGCCTAGG | 2014 |
| hs132686973 | AAGAAACCATTGCTGTGAGAGGG | 2015 |
| hs132685181 | GGAAGGGTGGAATAATGTCTAGG | 2016 |
| hs132684964 | CTAACCATTATAGAATTCCTGGG | 2017 |
| hs132693793 | CTACGCTCTTTGCAGCCTCAAGG | 2018 |
| hs132693442 | TCTGAGCAGAGATCAACTGCTGG | 2019 |
| hs132685167 | GCAATGCTATATAGATGAGCAGG | 2020 |
| hs132686564 | AGTCAATCAGCTGAAAGCCTTGG | 2021 |
| hs132689644 | ACCTGTATATGTAATTTCCAAGG | 2022 |
| hs132684686 | TACCTGTGGTCTAAAAAAGTCGG | 2023 |
| hs132687126 | TGCATATCCTCAGGGTTCATCGG | 2024 |
| hs132683740 | TCCTGGGAGCTAATCAAAAGAGG | 2025 |
| hs132692247 | GTAAATCCTCTCATTAAGATGGG | 2026 |
| hs132683998 | AGAAGTCAGTTCGAACTCCCTGG | 2027 |
| hs132688033 | GAAAGGATAATATAATGAGTTGG | 2028 |
| hs132689298 | AATATGGCAGATAAATTGCAAGG | 2029 |
| hs132686742 | ACTCTTTAACCAAAAGGCTGTGG | 2030 |
| hs132690235 | ATTCTGATAACCCCTTCGAATGG | 2031 |
| hs132684357 | GTGCCTACCAAGAGAAGAAGAGG | 2032 |
| hs132688499 | TGTGGGAAATCGGAGATGGCAGG | 2033 |
| hs132691122 | TGCTATCTAGGTTTAAGTGAGGG | 2034 |
| hs132692208 | ATTTTGATGTCATTACCATAGGG | 2035 |
| hs132693988 | TGAAAACAGTTACCTCCCATTGG | 2036 |
| hs132690075 | CTGATTTAGTAGACGCTGAAGGG | 2037 |
| hs132688823 | ACTTGGCAATCCAAGCAAGATGG | 2038 |
| hs132685997 | AAAGATTCATCCCAGTTTAGCGG | 2039 |
| hs132694348 | TATATGATAGTCTATTAACTTGG | 2040 |
| hs132688179 | TCCAGCTAGGACAGGCCTGAGGG | 2041 |
| hs132688585 | TATGGTACTGGGATTGTAGTGGG | 2042 |
| hs132691249 | ATTAGAAACACAACACAGCCAGG | 2043 |
| hs132691568 | ATAATGCACAAATATAAAGAAGG | 2044 |
| hs132687491 | GGTAAACGTGGCAGAAAGCAGGG | 2045 |
| hs132685835 | TATGCCTTAATTTCAGCCTGTGG | 2046 |
| hs132691853 | GCTATTCCAGGGGCCAGATGGGG | 2047 |
| hs132691260 | AACAGAATTCACTATCTCTAGGG | 2048 |
| hs132687383 | ATATTTACTGAACATTACATTGG | 2049 |
| hs132693696 | CTTTCTAGCTGAGGATGAGTAGG | 2050 |
| hs132694133 | AACAAGAACTAAATTTGCCAGGG | 2051 |
| hs132687517 | GGTTCTGTTTGACTTTTGAAAGG | 2052 |
| hs132693918 | ATGTATGGCAGAAGAACTAAAGG | 2053 |
| hs132687316 | TGAGCTCTGTTTGATGAAGGAGG | 2054 |
| hs132692288 | AATACAAAACAAACACAAGTAGG | 2055 |
| hs132687432 | GCTTCTAGTTTCACTAGGAACGG | 2056 |
| hs132685218 | CATAACTCTGACATAATCGTTGG | 2057 |
| hs132685990 | ACTTACCTAGTTATTTACTCTGG | 2058 |
| hs132684559 | AGTCCTTTCTACAGTTCCCAAGG | 2059 |
| hs132691031 | CCCAGGAAACAACTTGAAAGAGG | 2060 |
| hs132688626 | CTATCTTCTAGAGAGTTGACAGG | 2061 |
| hs132693149 | TCTCAATTAGTACAAAGCCATGG | 2062 |
| hs132685010 | ACATATTCAAAAATAGAGGTTGG | 2063 |
| hs132688965 | AATTAGTTCTTGCCTAGGGAAGG | 2064 |
| hs132684952 | CAGTGGTTAGGAGGAAGGAGAGG | 2065 |
| hs132685071 | TTTTCTAAGTGGACTTAGGAAGG | 2066 |
| hs132692188 | TGTGACTGATATGAATTCAAGGG | 2067 |
| hs132692622 | GCTTGATAGTATTTCACCACAGG | 2068 |
| hs132686570 | TTCAGGTTCTCGATGATGCAAGG | 2069 |
| hs132690939 | TCGCTAAGCTTGTCACCACATGG | 2070 |
| hs132691328 | GGAAAATTGCAGAATGCAGAGGG | 2071 |
| hs132683393 | TCATCCAGCACCATTAAAGAAGG | 2072 |
| hs132684347 | CTAACTTACCTTACTTGCAAAGG | 2073 |
| hs132686623 | ATAAATGGAGTAAAAGTTGAGGG | 2074 |
| hs132692501 | AACGTTATAGGCCCATAAAAGGG | 2075 |
| hs132691356 | TATCAAATTTGTTAAACAGGTGG | 2076 |
| hs132690831 | AAGATTGCGAACATAGCAAAAGG | 2077 |
| hs132689646 | GTTTTCAAATTTAAGCCAGAGGG | 2078 |
| hs132685202 | AGTGTGCTTCTTATGTCCTGAGG | 2079 |
| hs132693276 | AATCCAAACAGTGTTCATATGGG | 2080 |
| hs132686024 | GTGCCGTGCTACTTAAGAGAAGG | 2081 |
| hs132690720 | GTATAATCTATTTGAGGGCAGGG | 2082 |
| hs132691916 | AAACTTCATGTACCTTTCAAGGG | 2083 |
| hs132686802 | TTATTGCTGAAGCATGTGATAGG | 2084 |
| hs132688588 | CTACAATCCCAGTACCATACAGG | 2085 |
| hs132693975 | ACCTCAGAGGTTACAAAGGAGGG | 2086 |
| hs132687014 | ATATAAACTAAATTTTTCCAAGG | 2087 |
| hs132689086 | GAATCATTATTATCAACAGAAGG | 2088 |
| hs132688713 | GATCACAAAATATCTCTCAAAGG | 2089 |
| hs132684754 | TTCATGGTCCTCGATGCTGAGGG | 2090 |
| hs132689459 | TTTTTTCCCCTGAGTTACTGAGG | 2091 |
| hs132689411 | TGCAATGTGTGGTATATGAGTGG | 2092 |
| hs132689239 | CAGAAATGAGAACACAGGGTTGG | 2093 |
| hs132687881 | TTCTAATATCCCTCTCCCAAAGG | 2094 |
| hs132692393 | TGGCATAATCATTTGCCCATGGG | 2095 |
| hs132688943 | CCCCATGTGGGATAACTACAAGG | 2096 |
| hs132691754 | GTAGGATCATCTGCCGCTGATGG | 2097 |
| hs132689162 | AGTGGCTTAGCTGTAAATAGAGG | 2098 |
| hs132688286 | GTAGACATAGAGCTCAGAATAGG | 2099 |
| hs132693904 | CTTTTTAGCAACCTATTCCAAGG | 2100 |
| hs132684664 | AGACACAGCTATAAGGAAAGAGG | 2101 |
| hs132688331 | TTAATGGGAATTGACAAAAAGGG | 2102 |
| hs132684785 | TTCTATTTTCAGGAACCTAAGGG | 2103 |
| hs132687277 | TTACTCTTATTGAGAAAGCTCGG | 2104 |
| hs132688951 | ATTTGTTACAAAAATCAAGATGG | 2105 |
| hs132692152 | AAATACACCTTGGAATATCAGGG | 2106 |
| hs132689050 | GTCACATGATGCAGGCAGCTGGG | 2107 |
| hs132689545 | GATGAGGAGAAGGGTAGGAGAGG | 2108 |
| hs132688058 | CATTTTTCAGAGAAGAAGAGAGG | 2109 |
| hs132687397 | TTATTGGAATTTGTTATGGAGGG | 2110 |
| hs132692166 | CATTTGCTTTGCCACTCACATGG | 2111 |
| hs132694172 | CTTTCCCTAGAAAAAAAATGTGG | 2112 |
| hs132686382 | GAAAACAATTTCTCAATCCACGG | 2113 |
| hs132689224 | GCTATTTTTAGGTTTGGGGTAGG | 2114 |
| hs132689065 | CATCTGATTTCAAATCTGCATGG | 2115 |
| hs132693409 | ATAAGGAGATACAAAACAGATGG | 2116 |
| hs132683612 | GTGTTAGATACAATGCAGGCCGG | 2117 |
| hs132692604 | CCTCTTATAAAGACGAGAGCAGG | 2118 |
| hs132685490 | GCCAAATAGGGATACCTCAAAGG | 2119 |
| hs132689108 | TTATAGTTTTATCTGGCCTGGGG | 2120 |
| hs132691252 | GGGAGCAGAAGAATTAGAAATGG | 2121 |
| hs132691945 | AGGGACAAAATAAAGTCAGTTGG | 2122 |
| hs132687630 | TGTGAAACATACCAAAGGAAAGG | 2123 |
| hs132687863 | ATAATTATGCCATATTCAGATGG | 2124 |
| hs132693255 | TGAACCTTCCCTCAATACTAAGG | 2125 |
| hs132694387 | CAGCTGGGTCGACATCAACTTGG | 2126 |
| hs132688354 | GTATTAAGCGAAGCCATGGCAGG | 2127 |
| hs132683483 | ACGTAGTGAAGAAATGCAATGGG | 2128 |
| hs132687373 | GATGAAGTTTGGACAATGGATGG | 2129 |
| hs132689255 | AGCAGCTGCAGAGTGTTCTGAGG | 2130 |
| hs132694048 | ATGCTAACCTGAAATTGTATGGG | 2131 |
| hs132685871 | TTAAGGTGCGTATAAACTCATGG | 2132 |
| hs132688144 | AGCTAAAGAGTAAATCAGGCAGG | 2133 |
| hs132690679 | GGTGTTTCTTACCTGATGGAAGG | 2134 |
| hs132692992 | CATCAAGGTGAATGGGAAAGTGG | 2135 |
| hs132692871 | GTAATGCTTTCTCTTGTGAAAGG | 2136 |
| hs132684100 | GAAAAAATGAGAGCTGAAGATGG | 2137 |
| hs132687125 | TGTTTTCATGCATATCCTCAGGG | 2138 |
| hs132691574 | AAATCTAAGTGGAAGTCCAAGGG | 2139 |
| hs132684870 | CCAGGAAAAGGGGTTCAAGAAGG | 2140 |
| hs132688525 | CTACTTTTTCAGGTATTGTGGGG | 2141 |
| hs132685698 | TATGTGTCTACGTTGCTGGATGG | 2142 |
| hs132687431 | TTTCAGCTTCTAGTTTCACTAGG | 2143 |
| hs132688321 | GCAGTATAGGCTTTAAAGTAGGG | 2144 |
| hs132683590 | GTTGGACTCAGAAACCAGATTGG | 2145 |
| hs132684788 | TGGTCCTCATCAAAGTCCGCTGG | 2146 |
| hs132688522 | AAGTCACTGATATTTAATAGAGG | 2147 |
| hs132693860 | GTACATCACAGCAAAATAAGTGG | 2148 |
| hs132692413 | CATTTTGCTGAATTTAATTGGGG | 2149 |
| hs132691384 | AATACCAGATAAATGTAGCTCGG | 2150 |
| hs132693393 | GCCACCTGCTCTTATGCGTAGGG | 2151 |
| hs132683833 | TAACCAGTAACCGCTAAAAGAGG | 2152 |
| hs132686940 | TCAGAAGCAGGCTTCCCTAAAGG | 2153 |
| hs132683993 | TATCTCCAACAACGGAAAAGAGG | 2154 |
| hs132683864 | ATATAAATATAAGGGAATGTTGG | 2155 |
| hs132684088 | CTCGAGCCCCGCGAGAAGGAGGG | 2156 |
| hs132689015 | TCTTAAAGACAGTATACAAACGG | 2157 |
| hs132692658 | ATTTGAGGGGATGTCACAATGGG | 2158 |
| hs132687952 | GTTACTTAGTAATTCTCTGTAGG | 2159 |
| hs132693744 | TATCAGCAGATCCATCTGCTTGG | 2160 |
| hs132684493 | TTTATAGTCCATCATTTGCAGGG | 2161 |
| hs132685888 | TTTCATGTAGTTGGGATCAAGGG | 2162 |
| hs132691502 | GAATTTTGTCACTTAGAGAAGGG | 2163 |
| hs132688155 | ATTATTTGCTAGCCTCATGTAGG | 2164 |
| hs132693269 | CATTCATTTGGCCTTTGGAGAGG | 2165 |
| hs132690641 | CCTAAAAGTTAGTGCACAAGAGG | 2166 |
| hs132694284 | TTGCCTGCTCTTCAGGTGCGTGG | 2167 |
| hs132685285 | GAGAAAGAACACCTGTGCATTGG | 2168 |
| hs132684017 | TTATCTGTGAATCACCCAATGGG | 2169 |
| hs132685976 | CCTCCCTAATACCAGTGAAAGGG | 2170 |
| hs132692731 | TCTTTTTTAAGGATGAAGGAGGG | 2171 |
| hs132687790 | GAGTTATTTTAAATGTATAGGGG | 2172 |
| hs132693607 | GAGACAGAAATGAAAAATCAGGG | 2173 |
| hs132694139 | ATCTTTTCTTGAGTAGCGAAAGG | 2174 |
| hs132684607 | ACTTTGGTACTATTCCATAGTGG | 2175 |
| hs132690417 | AAGTTTGAAGAACTTTTACCAGG | 2176 |
| hs132688662 | AGTGTAAATAAACCAACTGATGG | 2177 |
| hs132689388 | AAACATTTCTTGCCATTCAAGGG | 2178 |
| hs132687980 | TATTTCAGAGAAAAAATATGTGG | 2179 |
| hs132690364 | AAGGCAGAAGTTTTGCCACTGGG | 2180 |
| hs132685207 | AGTTCTGCCAGGTTTATGTACGG | 2181 |
| hs132691660 | TTAATTAAAAGGAACGAAAGTGG | 2182 |
| hs132685788 | AGGGATACTTAACCAAAAGCTGG | 2183 |
| hs132687358 | ATATAGGAGACGTAATATATGGG | 2184 |
| hs132694353 | CTCAGGCTATGGAGGAGACTGGG | 2185 |
| hs132690712 | AAAACAATGGAACTGGGCCGTGG | 2186 |
| hs132683801 | CTAGGCCAGATAAGGAGCCCTGG | 2187 |
| hs132692283 | TCAATCTTAGCACCAGTGCCAGG | 2188 |
| hs132687750 | TATAGAATAGCTATATATTGAGG | 2189 |
| hs132684404 | GGAAGTTAGGAGGAGCAAGCTGG | 2190 |
| hs132692948 | CATCCCCTGCTTATTAGGATAGG | 2191 |
| hs132689955 | CCCATTCTCTAGACTTGCATAGG | 2192 |
| hs132693599 | ACACTCATCATGGGGGAGGAGGG | 2193 |
| hs132685208 | GTTCTGCCAGGTTTATGTACGGG | 2194 |
| hs132689659 | TATTACAATAATAAATTCAAGGG | 2195 |
| hs132687516 | CACTGTCCCTTAAAGCTGATCGG | 2196 |
| hs132689223 | CGAGCCTCATTCTTTTGTAGTGG | 2197 |
| hs132692753 | AACTTCCGTAACAATCTTTGGGG | 2198 |
| hs132684943 | TCTCCCCGATGCCTTAATCTGGG | 2199 |
| hs132690748 | TTAAAGAAAGAAATTAGGAAAGG | 2200 |
| hs132683564 | GGAAATGAATTGGTGACAGAAGG | 2201 |
| hs132687033 | ATTATAAGCATGGACCGTGCTGG | 2202 |
| hs132688297 | AACAACTTCAGATTAGTGTAGGG | 2203 |
| hs132684614 | TCTACTTAAAAAGGTTAAGGAGG | 2204 |
| hs132693177 | GCTTTCTACCCTAACAATAGAGG | 2205 |
| hs132691423 | TCTCCTTTTGTCTTTTAGTGGGG | 2206 |
| hs132685680 | GATTCTGGCTACTGTTTGAGAGG | 2207 |
| hs132689456 | ACAGCAGTCCTCAGTAACTCAGG | 2208 |
| hs132685042 | CTTAAAATGTTAAGTACTGAGGG | 2209 |
| hs132683345 | TATGTTTTTAAGAGAAGCAACGG | 2210 |
| hs132683574 | GTAGCAATATTTCAGTTCCAAGG | 2211 |
| hs132684477 | GTTTGTGCCACTTTAAACACTGG | 2212 |
| hs132693252 | AGGAACAACAGAGCATTCAATGG | 2213 |
| hs132690659 | CCTACCATTTCAGTCTTGGATGG | 2214 |
| hs132687221 | TATTACACTTTGTTGTCCGCAGG | 2215 |
| hs132692285 | TGAGATTCATCTCCTGGCACTGG | 2216 |
| hs132684805 | CGTTACAGGATACTGTAAAAGGG | 2217 |
| hs132686930 | GAGTCATCCAAATATATCCCAGG | 2218 |
| hs132686975 | CAGGTAGATTGAGCCGGTGAGGG | 2219 |
| hs132685284 | TCCCAGTGAGTCTAACAGCCAGG | 2220 |
| hs132691129 | ACCAATCTAAACATTTCCTAAGG | 2221 |
| hs132686227 | ACTTGAGGCCATGAAATTGAGGG | 2222 |
| hs132689606 | TCACTGTCTAAGCTAGTACTAGG | 2223 |
| hs132684457 | ATTTATTTAGTGCCCGGAAGGGG | 2224 |
| hs132685137 | CTTGGAATTCCTATGCCAGCAGG | 2225 |
| hs132686880 | CAGCTAGTTTTTTTTTCAGGGGG | 2226 |
| hs132691416 | AGCGTTTTCTCGTGTTCAAGAGG | 2227 |
| hs132686586 | CATTAGTCATGGAGTCCTAAGGG | 2228 |
| hs132691678 | CAGAAACGCTCTTTCAATGGTGG | 2229 |
| hs132683616 | AAATATATTACAACTTCCCGTGG | 2230 |
| hs132685796 | TGATGTTATCTATTTAGTACGGG | 2231 |
| hs132683664 | ATTGCTAAGCATGCCCTGTAGGG | 2232 |
| hs132692543 | GGGTGATAGTCGCCAGAGAAGGG | 2233 |
| hs132684142 | TCCAAATGATCAATTTTCCATGG | 2234 |
| hs132692107 | GGGTAGATACATTTTATGAGTGG | 2235 |
| hs132691083 | TATGGTGTGCCACTTGGAAAGGG | 2236 |
| hs132691691 | CATTTTATAGAAGTTGAACATGG | 2237 |
| hs132686125 | TTATCTCAAGGAATAAATAGAGG | 2238 |
| hs132694249 | AGAATGGGAGGATAATAGGATGG | 2239 |
| hs132688767 | TGTATAAGTATGTTTGGCAGTGG | 2240 |
| hs132684283 | GATGCACATTGATTTAACTTCGG | 2241 |
| hs132686309 | GATTTCAAACAGAAACCAGCAGG | 2242 |
| hs132686569 | GCAAGGAAATTACAGACGTTAGG | 2243 |
| hs132684981 | ATTTCGTTGACCCTTAACTAGGG | 2244 |
| hs132685474 | CAGCTGTGTAACATAAAAGCTGG | 2245 |
| hs132692108 | CTCATTTGCATGGCTCTACGTGG | 2246 |
| hs132683508 | ACATTGACTGTAAAGCGAATGGG | 2247 |
| hs132687959 | CCTTGTCTGTGTTACAGCAATGG | 2248 |
| hs132689858 | ATAAGCTCTTGGTTTTCTGAAGG | 2249 |
| hs132691494 | AAGCTGTATAGAATTTAAAGTGG | 2250 |
| hs132690360 | TTTTAATGTGACTCAATACATGG | 2251 |
| hs132690694 | TTTAGCTCTCAAAAGCACATTGG | 2252 |
| hs132692368 | ATATAAGAATGACATTTACTAGG | 2253 |
| hs132684035 | AATCTTACGCCTTTTAGAAGAGG | 2254 |
| hs132690391 | GAGTTGGGACGGATAATTGAAGG | 2255 |
| hs132683863 | TCTCGCAGTTGAAGGATTCAGGG | 2256 |
| hs132687764 | GCTCACAGTCGCCCCTATGAGGG | 2257 |
| hs132692350 | CTATAGCCTTGACTCACCACAGG | 2258 |
| hs132688472 | ATGCACAGAAAATAAAATGAAGG | 2259 |
| hs132691629 | TTATTCTGTTCTAGGATGAGAGG | 2260 |
| hs132692817 | GATCCTTCCTCTCATAAATGAGG | 2261 |
| hs132687223 | TGTTTGGGATTTCAGTTGGAAGG | 2262 |
| hs132691075 | CCCATGACAAAACTTTCAGCCGG | 2263 |
| hs132687700 | CATTCGGTTAATTTTCAAAGAGG | 2264 |
| hs132692848 | ATTTCTATAATCTATTGCCAAGG | 2265 |
| hs132687261 | GGTAGCTTACTGCTATTTGGAGG | 2266 |
| hs132687070 | GCCGCCCTCGAGCTAGGACATGG | 2267 |
| hs132690475 | AACTTTTGATTGAATCCCAAGGG | 2268 |
| hs132687275 | AGCTTTCTCAATAAGAGTAAAGG | 2269 |
| hs132692615 | TCAGCGATACTACCTGGGTATGG | 2270 |
| hs132692914 | TTTCATCTCTGTAGATGAGGTGG | 2271 |
| hs132689779 | CCTATAGTGCATGCATGCCAGGG | 2272 |
| hs132694100 | TATAAGTCAGAGGGAGGAGATGG | 2273 |
| hs132686693 | ATACTGCATGGAACTCGGGTGGG | 2274 |
| hs132686592 | TTACATCCTATACTTGGCTGAGG | 2275 |
| hs132688066 | AATAGTGTGGGTCAACTGAAGGG | 2276 |
| hs132690110 | ATGGAATCAGGACTATAAAAGGG | 2277 |
| hs132684189 | TCTGAAACACACAACTTGGATGG | 2278 |
| hs132693342 | TCAGATTATCTGCAACCCACTGG | 2279 |
| hs132691080 | TGCAAATGATAGACTCAAGAAGG | 2280 |
| hs132693455 | TGTCTTCTTGTTAAGCGAAGTGG | 2281 |
| hs132687192 | GTGCAATGCCCAGAGCCATAAGG | 2282 |
| hs132693345 | ATGTTCTGAAAGAACACAAAAGG | 2283 |
| hs132689938 | ATACTATTCTGAATAGTACTTGG | 2284 |
| hs132692777 | TTTGTAATACAAATTACGATGGG | 2285 |
| hs132694204 | ATTTCAGTAGACTTACAGAAAGG | 2286 |
| hs132683638 | ATATTGAGTAGGCAGCTGTATGG | 2287 |
| hs132691706 | AAATGCAGCTGGAGTGAGTAGGG | 2288 |
| hs132685432 | GAGCAATGAGTATGTAGGAAGGG | 2289 |
| hs132693439 | CCATAACCAAATGGGATGAAAGG | 2290 |
| hs132688846 | TGCAGGGTCTGGGGATTCTGGGG | 2291 |
| hs132692497 | CCTGTAACTGGAGACAAGAAGGG | 2292 |
| hs132688611 | AGTCTACCTGGATTATTGATGGG | 2293 |
| hs132690262 | CGTTTTTATTAAATGCAACCAGG | 2294 |
| hs132684566 | AGTTTGGAGCTCCATTTTCAGGG | 2295 |
| hs132689810 | TATATAAAGTTTCTTGGTGAGGG | 2296 |
| hs132693891 | ATCTACTCTGCATGAAGCTAGGG | 2297 |
| hs132693287 | GACTGGGGAATATAAAGAAAGGG | 2298 |
| hs132689397 | GATCCTTAAAGAGAAAACTTAGG | 2299 |
| hs132688237 | CAGACCTTTGACACTTACTTCGG | 2300 |
| hs132685234 | CAGGCCATCAAACACCCATAAGG | 2301 |
| hs132690189 | GAAGATGTGAGCTATAAAAGAGG | 2302 |
| hs132688639 | CAAATGATCTTTATGCCCTAGGG | 2303 |
| hs132684131 | TATAAACCACACATCACGTATGG | 2304 |
| hs132694305 | GGAAGCTCACATTTTTTCGAAGG | 2305 |
| hs132693857 | ATAGAGAATTCATGGCCCTCAGG | 2306 |
| hs132691224 | ATATTCCACCTTGAAAAAGCAGG | 2307 |
| hs132691043 | TGGAGGCCTTATCAAACACCAGG | 2308 |
| hs132694171 | TTTCTGCTCACTGGTAGCGCTGG | 2309 |
| hs132688042 | ACTTTCTTTTGCTAAGTAGGAGG | 2310 |
| hs132685873 | GATTCTACTTGTATCATGATGGG | 2311 |
| hs132687031 | ATATTTGGCAATTATAAGCATGG | 2312 |
| hs132691868 | TTAGAGACCGATGAGTGAAAGGG | 2313 |
| hs132684827 | GTTCTATCTGAATAAAGCCATGG | 2314 |
| hs132692510 | GCTTTTTCCTTTGAGAACTGTGG | 2315 |
| hs132686212 | GATGTGTTCAAAACATTATGAGG | 2316 |
| hs132687419 | TCATGCCAAGTTAAAGTGGTTGG | 2317 |
| hs132687035 | CTACTCTCAATTTTCAGATGCGG | 2318 |
| hs132684021 | ATTGTTTCCATCTAAAGTAAGGG | 2319 |
| hs132693051 | ACTGAAGGACAGCGAGTGCATGG | 2320 |
| hs132689263 | GAATGCTTATAAAGAGTGGCAGG | 2321 |
| hs132685975 | TCCTCCCTAATACCAGTGAAAGG | 2322 |
| hs132685346 | TGTTTTGCTTGAAGGAACTCAGG | 2323 |
| hs132686099 | CATTCCTAAGCATATTAAAGAGG | 2324 |
| hs132692026 | CCAAGACTGAATCATAGAATGGG | 2325 |
| hs132686302 | TTTAACATAAGGCTTTAGACTGG | 2326 |
| hs132692866 | CATGGATGTCTCTTGTCATACGG | 2327 |
| hs132683636 | ATTTCACCTCTAAATTACACTGG | 2328 |
| hs132688434 | GGAAGGAGCAGCAGTTCTGTGGG | 2329 |
| hs132685824 | ATATAAAAGGATTCAGTATGAGG | 2330 |
| hs132684673 | TTTGAAAATGGGTCTGAGAACGG | 2331 |
| hs132687215 | TCAAAGAGTACCATATAAGCGGG | 2332 |
| hs132683650 | ATGCTTTAGAAACAGACAAATGG | 2333 |
| hs132687055 | TGCTTAGAAAGATTAGTTGAGGG | 2334 |
| hs132686761 | GAGAGGGAGAGAAAGAGAAGTGG | 2335 |
| hs132687029 | TATTAAAGAAAACAAGGTGTGGG | 2336 |
| hs132692809 | ATATTAAGTATGTGTCCAAAAGG | 2337 |
| hs132691033 | GGAACAGAGGCGTAAATAGCAGG | 2338 |
| hs132684737 | ACAATGTCGTTCTTGAGGGATGG | 2339 |
| hs132687202 | TGCTTTATTGGAGGATCACAGGG | 2340 |
| hs132688897 | TCCTTGTCCATCCCAGTACAAGG | 2341 |
| hs132689766 | TTACTGAGAAAGTGAATGGTTGG | 2342 |
| hs132690934 | TCTCAGTGATTCTTTCGCCCAGG | 2343 |
| hs132692546 | CTTATGGATTGTTTGACTGGAGG | 2344 |
| hs132690063 | TCTGACAGCCCATTAGAGACGGG | 2345 |
| hs132688982 | GTTGAAGTGGGCATGTGGAAGGG | 2346 |
| hs132687129 | CCTCCAAGCTAAAAATTCATGGG | 2347 |
| hs132684713 | GGCATCAATAGCTAATTCTAGGG | 2348 |
| hs132685381 | TCTTTTTTCAAAATTATGCCGGG | 2349 |
| hs132686315 | AAGAAGCAGAAGAAACAAACCGG | 2350 |
| hs132692157 | TTATATGTTAACATGATCAAAGG | 2351 |
| hs132683523 | ATATTATCGATTAGGCCTGTTGG | 2352 |
| hs132692788 | GACAGTGGACCACATTGTATAGG | 2353 |
| hs132684165 | AATTTGCAGCCTTTTGTAGAGGG | 2354 |
| hs132686903 | AACTCATTAGAAAACTAATGAGG | 2355 |
| hs132685935 | TGGCTCTGGAGATTTTATAGAGG | 2356 |
| hs132693811 | CCATGCTGCACAGTACAATTGGG | 2357 |
| hs132688685 | TTAGAGTTCTCAGTAAATGTAGG | 2358 |
| hs132690330 | GCATAATTACATTATTTAAGGGG | 2359 |
| hs132692662 | GAGGCCATTGAACAATTTGAGGG | 2360 |
| hs132685009 | GATTCAAATGGCACAACAGAAGG | 2361 |
| hs132684904 | TTTCACATAGTGGTTGGTGATGG | 2362 |
| hs132685035 | ACATGCATATTTTACTGCCATGG | 2363 |
| hs132689027 | TTCTGATTCCCATTCTCTGAGGG | 2364 |
| hs132686380 | ATTCTACTATTAAGTAATGAAGG | 2365 |
| hs132689586 | TTAGTAAAACTTATTTGCGCGGG | 2366 |
| hs132684584 | ATTTCTGCCATTAATACTGTAGG | 2367 |
| hs132693985 | GACGTACATAAACTACCAATGGG | 2368 |
| hs132689428 | GGTATTACAGAAGCATTGTAAGG | 2369 |
| hs132688412 | CTGCTTTTAGAAAGTAAGCCCGG | 2370 |
| hs132692626 | TCTTCTTAAGCAGCTAGTATGGG | 2371 |
| hs132693770 | TAAAACGGCATGAAAGGAAAGGG | 2372 |
| hs132692648 | GTTTTATGTTGGGTACAGGTTGG | 2373 |
| hs132685204 | ATTCTGCCTAAAGTTCTGCCAGG | 2374 |
| hs132685291 | ATTGGCAGTGACAGCGGAGGAGG | 2375 |
| hs132689856 | TCTTGGTTTTCTGAAGGCTAGGG | 2376 |
| hs132690312 | GAAAATATTACAATAGAAGGTGG | 2377 |
| hs132688969 | TATTGTGAAGGAGCAAATACCGG | 2378 |
| hs132692432 | CCTCTCTCATGGACAGTCAGAGG | 2379 |
| hs132691760 | GAGCAGGACTGTCTAGACTTTGG | 2380 |
| hs132689442 | GTCCGAAAAGAGCAAAGGGCTGG | 2381 |
| hs132683598 | GCATAAATTCTGTGATTAAGTGG | 2382 |
| hs132693723 | AGACTGAGACTGTATCTACTTGG | 2383 |
| hs132684416 | TATTCTGCCTCCAGGATTTGGGG | 2384 |
| hs132686922 | GGGGCTTTTGTTGTGAGGAAGGG | 2385 |
| hs132687245 | GTTAGGCTCCTGCTGAGAGTTGG | 2386 |
| hs132683992 | ATCTCCAACAACGGAAAAGAGGG | 2387 |
| hs132688229 | GATTGATAAATGAGAGTCCTGGG | 2388 |
| hs132690734 | AACTACATTGTGAAGCCTGATGG | 2389 |
| hs132685569 | CACATAACCTCCCATTTCTAGGG | 2390 |
| hs132683692 | TTAGAACTTCTTGAACAAGTTGG | 2391 |
| hs132690183 | AAATACTTGGGAATTTCAGTAGG | 2392 |
| hs132683387 | AAGATAATCAGAGTCACAAAAGG | 2393 |
| hs132685507 | TTAATATGTAACTCATGGCTTGG | 2394 |
| hs132686134 | TAACACCTGAGTTTTTAAAGTGG | 2395 |
| hs132691600 | TGAGCTGTTACAAAAGTGGTTGG | 2396 |
| hs132686133 | GTATTCCACTTTAAAAACTCAGG | 2397 |
| hs132689627 | GTTCATCAAAATACTCGTGTTGG | 2398 |
| hs132690141 | TAATGAAATGAGAATGTAGATGG | 2399 |
| hs132684922 | TCTAGAACAGCATTTCCAAATGG | 2400 |
| hs132688270 | GCAAGACCAAGATTATCATCTGG | 2401 |
| hs132693926 | GCAGGTACAGAAGTAGATCATGG | 2402 |
| hs132692122 | GACAGTGGTGAGTTTTAGTAGGG | 2403 |
| hs132684715 | CTCATATAGTTTAAAACACACGG | 2404 |
| hs132685494 | TTTTATTCAGTAGATATCACTGG | 2405 |
| hs132693175 | AAGCAGCGCCTCTATTGTTAGGG | 2406 |
| hs132693713 | ATTACAGTATATTCTACAGTTGG | 2407 |
| hs132694225 | CTTTTCCATTGCTACCAAAGAGG | 2408 |
| hs132694207 | GCTGAATTTACCTATTTGGTAGG | 2409 |
| hs132692488 | ATATTGCAGCAACATTATGAAGG | 2410 |
| hs132687401 | CTATAAACTTTAAGGGAGCAGGG | 2411 |
| hs132690421 | ACCTTCGGAGAAAAACGAATAGG | 2412 |
| hs132688267 | ACATTTCCTCTTGATAAATGAGG | 2413 |
| hs132683681 | AAGATTTCCAGTCTGAAAAGAGG | 2414 |
| hs132686066 | GAGAAAGGATGTTTTAGCTGTGG | 2415 |
| hs132693113 | ATCCCATATTCCTATTACCCAGG | 2416 |
| hs132688269 | ATAATCTTGGTCTTGCCTGCCGG | 2417 |
| hs132689151 | GGAAAAATACTTGGCCAGATGGG | 2418 |
| hs132690134 | ATAAATATGAAAACCATACATGG | 2419 |
| hs132694343 | GTCTGGTTAAGGAACACTATTGG | 2420 |
| hs132687849 | AATATCAACCGTCTTCTCACTGG | 2421 |
| hs132692302 | TGCTAGAAATTTGTGAGCAAAGG | 2422 |
| hs132688195 | ATTCTGAGCATGCCCAGTGGTGG | 2423 |
| hs132689768 | CAGATTACTGAGAAAGTGAATGG | 2424 |
| hs132687151 | TTTAATTATACAGCTACCCTGGG | 2425 |
| hs132687050 | CTAATTATACTCATGCTACAAGG | 2426 |
| hs132690737 | GATGCCACCTAATCTCCATCAGG | 2427 |
| hs132690062 | CTCTGACAGCCCATTAGAGACGG | 2428 |
| hs132686263 | TATGAAAAAAAAATAGGATAGGG | 2429 |
| hs132689867 | CAGTATGGGAAATCAAAAAAGGG | 2430 |
| hs132688428 | TCAGTGAGTTTATTAGGCTGAGG | 2431 |
| hs132685180 | TAGCTAAGTATTAGGAAGGGTGG | 2432 |
| hs132687058 | TGTTTCACATTTAAGAGGGAAGG | 2433 |
| hs132685141 | TGCTGTATGCCTGCTGGCATAGG | 2434 |
| hs132684440 | ATAATTTAGTGCAAAGGTGTAGG | 2435 |
| hs132684571 | AATTGAGTGGTGCACGTCGAGGG | 2436 |
| hs132693012 | ACTTTAAAGTCGTTGTGAAGTGG | 2437 |
| hs132690697 | AGGAGTTTAGTGATATCAGTTGG | 2438 |
| hs132692565 | CCATCATTCCCATGGCCTGTAGG | 2439 |
| hs132694051 | TCGGTCCAGCAACAATAAGCTGG | 2440 |
| hs132691722 | GCTCATTACATTCTATATTGTGG | 2441 |
| hs132691011 | GCTAATTAATGGGGACCCATGGG | 2442 |
| hs132693719 | TATTTGATGCCGTATGTGCTCGG | 2443 |
| hs132685427 | GGTCTATTCATTCAGGCTAAGGG | 2444 |
| hs132692801 | TATATGTTTAGCCTAGAGGCAGG | 2445 |
| hs132685998 | GTTTTCTCTTCCGCTAAACTGGG | 2446 |
| hs132690917 | AGTATGAGCATGCAACGGTTAGG | 2447 |
| hs132687515 | CCCTTAAAGCTGATCGGCAAAGG | 2448 |
| hs132692127 | CGTTGCACACGTCCTCAACTAGG | 2449 |
| hs132689751 | CCAGGGTGGAAAATAAGCTTAGG | 2450 |
| hs132694149 | TCAGTTTTATAGTCTTCAGTGGG | 2451 |
| hs132688557 | TTGAGAGTAAAATTTGAGCATGG | 2452 |
| hs132693398 | GCCATTTGCCAAGTGCCAAATGG | 2453 |
| hs132690286 | ATACAGGGTATATAATTTGAAGG | 2454 |
| hs132692203 | CATTGTGGGATTCCTCCCTATGG | 2455 |
| hs132693459 | TAAAAAGTGGAGCTTGCAAAGGG | 2456 |
| hs132687579 | TTTCTTGTTCATCTATTGTGAGG | 2457 |
| hs132690116 | GCTTTTGGCTAAGACTGTCATGG | 2458 |
| hs132689553 | TTGTAGCCATGATAAACAAATGG | 2459 |
| hs132685221 | TTTACATTTCATTCTTTAGGGGG | 2460 |
| hs132693171 | GAACCATTAGTACTTACAATGGG | 2461 |
| hs132687207 | TTTCAAAATTGGCTATTCTGTGG | 2462 |
| hs132689019 | AGATGTATTGATTTCTCCAAGGG | 2463 |
| hs132689400 | GTTTTCTCTTTAAGGATCTGTGG | 2464 |
| hs132692458 | CCTCTGAGCCCTCAAGGTGGAGG | 2465 |
| hs132685852 | TTATGCAAAGCTCCTATTGTGGG | 2466 |
| hs132689952 | CCTATGCAAGTCTAGAGAATGGG | 2467 |
| hs132685115 | CATTTTATACTAACTATGGGTGG | 2468 |
| hs132693028 | GCACCCATGTCAATATGATCAGG | 2469 |
| hs132690252 | ACTAATAGATGTTTTACGTAGGG | 2470 |
| hs132685214 | TGAAAACCTGGAAAAACTAAAGG | 2471 |
| hs132687779 | GGAAATAACAGACAAAAGAAAGG | 2472 |
| hs132685893 | AGACAGATTTTAGGTTAAAGGGG | 2473 |
| hs132694286 | TTTCATTAATAATCAACAGCTGG | 2474 |
| hs132692430 | GAGGCCAGACTTGCAGGGCAGGG | 2475 |
| hs132686694 | TACTGCATGGAACTCGGGTGGGG | 2476 |
| hs132691764 | AATTCAGCCTGGTATTACTCTGG | 2477 |
| hs132684234 | CTTTCACTGTGGTAACTGGAAGG | 2478 |
| hs132685786 | TATCAAGATACTTGAAAGCAGGG | 2479 |
| hs132692462 | TTGTGAACTCCTCCACCTTGAGG | 2480 |
| hs132688314 | ATAATTAGCTTATGATCAGATGG | 2481 |
| hs132685193 | CCACATTGCTGGTACCGTTGGGG | 2482 |
| hs132684454 | ACATGTATTTATTTAGTGCCCGG | 2483 |
| hs132686770 | CTTGGAATTGAAATGGAAGCTGG | 2484 |
| hs132687675 | ACATAATACAAACTTCACTTTGG | 2485 |
| hs132685350 | AAGGTCCTCTTCATAGCAGCAGG | 2486 |
| hs132683673 | TTATTGTCTGCTCTCTGTAGTGG | 2487 |
| hs132683410 | CTTTCTGCTACCAGTAGAAGTGG | 2488 |
| hs132686894 | TTTCAGATAATTAGGCAGCCTGG | 2489 |
| hs132689648 | AGAGGGGAAATATACCCTAATGG | 2490 |
| hs132685729 | GATTTCACAAATTAAGGAGAAGG | 2491 |
| hs132690403 | TGCATATTCTATTACTGCAGTGG | 2492 |
| hs132686514 | GTAGAAAACTGTGTCATTCGTGG | 2493 |
| hs132684679 | TTATGGGTGTAAAGTGTAAGTGG | 2494 |
| hs132691339 | CTACGTTGTTAAAAATGGGCAGG | 2495 |
| hs132693847 | TAGAGGGAAGAAGGATGCAATGG | 2496 |
| hs132691682 | ATTATCATAATTGAATAAGAAGG | 2497 |
| hs132693116 | TATTTTAGATTTGCTTGTAACGG | 2498 |
| hs132688752 | TTCCAACTCTACACCTCCATTGG | 2499 |
| hs132692266 | TTTGACTTTTTATATTTGAAGGG | 2500 |
| hs132694014 | CCAGACAGTAAAGCTGACTTAGG | 2501 |
| hs132692101 | AGCCCTGGTGATTTTTAGGGTGG | 2502 |
| hs132693853 | CTTTGCTTAGCTCTTCTGGAGGG | 2503 |
| hs132691214 | TTTAATTTCAGGGTTCATGAAGG | 2504 |
| hs132693298 | TTTTAGAATAGGTTACAAGTAGG | 2505 |
| hs132692252 | CAGTAGATCATGTTATGTAAAGG | 2506 |
| hs132686131 | GAGATATTACCCCAGAAGGCTGG | 2507 |
| hs132688583 | GGTCAAAATCAAGATTTGGCAGG | 2508 |
| hs132693196 | ATAGAGACTTCCAAAAAGAATGG | 2509 |
| hs132689508 | CCGCCAATGGAGGTTAGGTATGG | 2510 |
| hs132690113 | GCCAAAAGCAGAGTGGAAGAAGG | 2511 |
| hs132684739 | TGTCGTTCTTGAGGGATGGGTGG | 2512 |
| hs132684061 | AGTGTCATACCTTCCATCAAAGG | 2513 |
| hs132683674 | GACAATAACGATGCTCCTATGGG | 2514 |
| hs132685463 | TGAGGTAACCAGCCAGGCTACGG | 2515 |
| hs132689279 | TGATTTCCATGAGGAAAGCTGGG | 2516 |
| hs132687793 | TCAAAATTAGTTCCAGAAGAAGG | 2517 |
| hs132687171 | ATCTTCTTAAACATTTTCGTTGG | 2518 |
| hs132684592 | GTAAAAAATGAGAGATAGGTGGG | 2519 |
| hs132692720 | TTTATATTACTTGAGGGTGGTGG | 2520 |
| hs132693662 | GTATTATAACTCTTTAGCATAGG | 2521 |
| hs132691049 | TCCTAGCAAAAACTCCCAAATGG | 2522 |
| hs132688392 | TTATGGGCATGAACCAGCCTTGG | 2523 |
| hs132685636 | ATAGAGGGAGATAAAGGAAGGGG | 2524 |
| hs132685307 | GAAGCTTTTTCCCTTAAGGATGG | 2525 |
| hs132685085 | GTTAATAAATGAAGAACTAAGGG | 2526 |
| hs132683701 | CTCTTCATTTAAAAAAGAGAGGG | 2527 |
| hs132693408 | TGTTTTTATTATGCTAGCCATGG | 2528 |
| hs132684204 | TTTGCTATGCACCGGACCCTGGG | 2529 |
| hs132684814 | AATAATACCCACATCAGTAGAGG | 2530 |
| hs132688416 | AGAACACATCAGAAAATAAGTGG | 2531 |
| hs132687668 | CTATTGTTAAACATCAATAGAGG | 2532 |
| hs132692063 | ATTTCTGTAATAAAAAGTATGGG | 2533 |
| hs132692027 | GCCAAGACTGAATCATAGAATGG | 2534 |
| hs132686765 | CACAGAGGCTGAGTTTTGACTGG | 2535 |
| hs132690204 | TTTATATGCCCTTAAAACGCTGG | 2536 |
| hs132689472 | TGTGACCCGACTGGTTCCCTGGG | 2537 |
| hs132686636 | AGTGTCAGATATGGAGGTTGTGG | 2538 |
| hs132694058 | TTATCAGCATCATTCAGCTCTGG | 2539 |
| hs132686479 | ACTGAGGCTATACTTCTGTATGG | 2540 |
| hs132690835 | AGATTATAATTGTTTATAGAGGG | 2541 |
| hs132688303 | TGGCACAGTAAGCCTTTGGATGG | 2542 |
| hs132685237 | AGGATGACAAAGGTCCTTATGGG | 2543 |
| hs132691444 | CTAGTCCTCCCTGAGAATGTAGG | 2544 |
| hs132692641 | AAGAGCCTCCTCGAGGTGAATGG | 2545 |
| hs132688395 | GAGTATTACACACAAAATAGTGG | 2546 |
| hs132690622 | ATTTACCTCATAAAACAGTTGGG | 2547 |
| hs132693691 | TTGTTACATCTAATACTGGTTGG | 2548 |
| hs132686795 | CTTTAATCATGAAAACAAAAGGG | 2549 |
| hs132686101 | GTTAATGTCCTACTAAAGCTAGG | 2550 |
| hs132687553 | ATACCACCACTGTACTGTACTGG | 2551 |
| hs132687026 | ACGTAAGCCACAATTTTACAAGG | 2552 |
| hs132688756 | TTCACCTCTCTCAAGAGGCTTGG | 2553 |
| hs132687296 | TGATTGCATATCTTTCCCATAGG | 2554 |
| hs132685107 | GCTCGATTAGGTTTGGTGGTTGG | 2555 |
| hs132686025 | ATAACTTGAGTGCTTTTCAAAGG | 2556 |
| hs132683745 | CTTGGATAATCTATTAGCATTGG | 2557 |
| hs132694427 | TTTGCAGAGTTTGAAGAGCTCGG | 2558 |
| hs132692325 | TCTGTATATCTGCCTCCTCCAGG | 2559 |
| hs132686987 | TGAATACCAGACAAAAATACAGG | 2560 |
| hs132692855 | ATTGCATAACCAGTTGTCACTGG | 2561 |
| hs132687156 | GCAGCACAATGTATCACCTTAGG | 2562 |
| hs132691036 | GTAGTCATCTTTGTGGTAGGTGG | 2563 |
| hs132688652 | GGATTGTCTAAGTCATTGGCTGG | 2564 |
| hs132688313 | CAGTGAGAGTTGGATAGAGAGGG | 2565 |
| hs132692560 | TTCTTAGTAGCATACCACTATGG | 2566 |
| hs132685118 | ATTACAGCCAATCACAATAGTGG | 2567 |
| hs132685434 | ATAGAGAGCAATGAGTATGTAGG | 2568 |
| hs132690678 | TTAAGGTGTTTCTTACCTGATGG | 2569 |
| hs132691170 | TGCATGACTTGAAGACACTAGGG | 2570 |
| hs132685983 | GTGATCAGTGAAATTTACATTGG | 2571 |
| hs132691420 | AATCTGTCAGTATCTCTCTCTGG | 2572 |
| hs132692410 | GATTCGGTAAATGCCATTTGAGG | 2573 |
| hs132683700 | CCTCTTCATTTAAAAAAGAGAGG | 2574 |
| hs132686936 | TTGGTCAATTTGCAACCCCTGGG | 2575 |
| hs132691427 | GGGAAGGCAACTTAAGCTAAAGG | 2576 |
| hs132686830 | ATAGACTAAAACTTTACATAGGG | 2577 |
| hs132689281 | CTAAAACCCAGCTTTCCTCATGG | 2578 |
| hs132687011 | CCCAGAGCATAAGATTGAAGTGG | 2579 |
| hs132692088 | TTGTAGCTGTGAGTAAAGTAAGG | 2580 |
| hs132684388 | TAGTGCCCAACCAGAGCAAAGGG | 2581 |
| hs132691250 | AGAAATGGTGGCAAAGAGCTAGG | 2582 |
| hs132693230 | ATTGTTGTAAATTTTCCTGAAGG | 2583 |
| hs132687411 | CTTGGGAGAGCCCCGAAAGATGG | 2584 |
| hs132690221 | TAATCTCTAGAGGAGCCATCTGG | 2585 |
| hs132689153 | TATAAAGGTGGAAAAATACTTGG | 2586 |
| hs132687752 | TTATAGTTAGCAAGATAGCCTGG | 2587 |
| hs132685431 | TGCTCTCTATTATACAAAGAAGG | 2588 |
| hs132691963 | TTTTACATATATTGATTGGAGGG | 2589 |
| hs132685591 | ATTGGCATAGCATTACACTATGG | 2590 |
| hs132684475 | CCTTAGATAGTATTATTATGTGG | 2591 |
| hs132688202 | GTAGGCCTCAAGACCTTGAATGG | 2592 |
| hs132686242 | GTAGATGTCACAGCGCAAACAGG | 2593 |
| hs132688384 | GGAGAACCATGCAGTAGGGTGGG | 2594 |
| hs132683972 | CTCCCAGCTCAACTTTGGGATGG | 2595 |
| hs132685715 | AGGTTCTTTTACTTTTAGCAAGG | 2596 |
| hs132683985 | GTGTCAATCAAAATAGTCTTCGG | 2597 |
| hs132688755 | AGAGAGGTGAAGGATATCTCAGG | 2598 |
| hs132693159 | TCTCTGCTCCAATACATACTTGG | 2599 |
| hs132691116 | GTTAAAAATCTGCTAAGTAAAGG | 2600 |
| hs132690951 | CTTCAGCCCCACACCAAACATGG | 2601 |
| hs132692942 | ATCGTCAATGTTGAGCGTATCGG | 2602 |
| hs132690100 | TTTAAGTCAGCACTTAGCTCAGG | 2603 |
| hs132691833 | TCTTTTCAAGAATGATTGCTGGG | 2604 |
| hs132691424 | AGACCCCACTAAAAGACAAAAGG | 2605 |
| hs132686209 | AATGAGGAGGAAAAAATATGAGG | 2606 |
| hs132686585 | TCATTAGTCATGGAGTCCTAAGG | 2607 |
| hs132689025 | GATGTATGCCCTCAGAGAATGGG | 2608 |
| hs132687420 | GTCCTCATGCCAAGTTAAAGTGG | 2609 |
| hs132690023 | ATAGAGTGTGAAGAGGAGATCGG | 2610 |
| hs132686102 | TTATTCCCTTTTCTAAAATGAGG | 2611 |
| hs132683374 | TCATATTGAGCTTTTGGCAGAGG | 2612 |
| hs132689598 | AGAGCCACTGTTCTAAAAGCAGG | 2613 |
| hs132692555 | GATTGAGGCCTGATTCAACTTGG | 2614 |
| hs132683952 | ATCATTATAAGGAGATGAAAGGG | 2615 |
| hs132694421 | GCTGCGTTAGATTTTGGGGAGGG | 2616 |
| hs132693086 | TATTTCTGTCTGTTTGGAGTGGG | 2617 |
| hs132692234 | GCTCAACTGTGTGTTAGGCCTGG | 2618 |
| hs132691268 | GAGTCCAGTTAAAGGTAGCTTGG | 2619 |
| hs132685581 | GTAAGATAGTGTAAGTAAAGAGG | 2620 |
| hs132685416 | CTAAGCATCTTTTTTCAAGCGGG | 2621 |
| hs132691039 | GCAAAGTGTAGTCATCTTTGTGG | 2622 |
| hs132689887 | GAACAGATGTTACGTGGTAGAGG | 2623 |
| hs132686321 | AAGCTTTAGATGCTGTACTTAGG | 2624 |
| hs132685116 | TTACATTTTATACTAACTATGGG | 2625 |
| hs132686579 | TCCTTTGTCTCATATACTACGGG | 2626 |
| hs132690661 | CATCCAAGACTGAAATGGTAGGG | 2627 |
| hs132683943 | TGTCTTCTATGGCAAACCATGGG | 2628 |
| hs132688266 | AAATGTGAGTTTCTCTCAAAGGG | 2629 |
| hs132687561 | CAGGATAGCTTACTTATACAGGG | 2630 |
| hs132688650 | GTCTAAGTCATTGGCTGGAAGGG | 2631 |
| hs132688921 | TTTCTTTCTCAGCCTCCAAAAGG | 2632 |
| hs132694378 | TTTCTTTCCCCTACAATCAGTGG | 2633 |
| hs132694218 | CTACTTAAGTACTAGAGACTTGG | 2634 |
| hs132690339 | TTTGTAAGTTTATCAAACTACGG | 2635 |
| hs132693458 | CTAAAAAGTGGAGCTTGCAAAGG | 2636 |
| hs132684838 | ACCAGTCTTTTAAACAGTATCGG | 2637 |
| hs132687324 | ACTGAGTAATGGAATCTTGAGGG | 2638 |
| hs132693527 | TTTTAAGTCAACAACATACATGG | 2639 |
| hs132683840 | TTTAGTTTGACTCCTCAGCTAGG | 2640 |
| hs132688983 | GGTTGAAGTGGGCATGTGGAAGG | 2641 |
| hs132689133 | CCGCAGCTAATAATGACATATGG | 2642 |
| hs132688486 | TGATGTATAGGAAGCAAAGAAGG | 2643 |
| hs132685174 | CTCTGTAACTTGTTTTAAGGTGG | 2644 |
| hs132684546 | AAGTATTGCCAGAGCTTGAAGGG | 2645 |
| hs132692451 | TGAAAAGGAGTAGGAGGCTGAGG | 2646 |
| hs132692933 | GAGATATGTGCGGCTAACACAGG | 2647 |
| hs132689269 | GGAGCCAGAATAGGGAGCTAAGG | 2648 |
| hs132686100 | AGTGCCTCTTTAATATGCTTAGG | 2649 |
| hs132684438 | TCTAGCCTCCACTCTAGGATTGG | 2650 |
| hs132688302 | GCTGTGGCACAGTAAGCCTTTGG | 2651 |
| hs132690267 | ATTGGAATATTTTCTGGGGAAGG | 2652 |
| hs132685589 | AACTTAATATTTGCAAAACTGGG | 2653 |
| hs132693972 | CCTCACACCCAACTCCTCCTAGG | 2654 |
| hs132686576 | TTCCATAGTTGATTAGCGTATGG | 2655 |
| hs132689443 | AGGTGTCCGAAAAGAGCAAAGGG | 2656 |
| hs132693138 | TGTTTGTATTATAGTCCAAAAGG | 2657 |
| hs132691838 | AATCTGTTACACTAGAGATACGG | 2658 |
| hs132691770 | AGTTTTGTCCATATCATAGCAGG | 2659 |
| hs132683791 | GTGTATATCCATAAAAAGAATGG | 2660 |
| hs132685164 | TATCAAAACTAAGTGCAAATGGG | 2661 |
| hs132692563 | CTATGGCCCCATCATTCCCATGG | 2662 |
| hs132687514 | TGATCGGCAAAGGAAGAAGCTGG | 2663 |
| hs132691497 | TTAAATAGAGCAAGTATGCTTGG | 2664 |
| hs132683988 | TATGAAACCCCCATAGGCTACGG | 2665 |
| hs132684562 | TTGCAACCAAACAAATCCTTGGG | 2666 |
| hs132685863 | ATTCATGTATTATAAAAGGCTGG | 2667 |
| hs132693365 | AAGAGATTAATGGCAAGAAGAGG | 2668 |
| hs132686917 | GTTTGACACCAATAACCTATAGG | 2669 |
| hs132688382 | AGAACCATGCAGTAGGGTGGGGG | 2670 |
| hs132689608 | TCTGAGCAACATATTACAATGGG | 2671 |
| hs132689076 | TTTCAGAATAATAAATAGTAGGG | 2672 |
| hs132685450 | ATATATCTTTCAAAAATAGCTGG | 2673 |
| hs132683407 | GGTAGCAGAAAGAATGAGGTTGG | 2674 |
| hs132688975 | GACATCTGAGGAGGAACAAAGGG | 2675 |
| hs132692175 | ATATGCATATAAGATGTCATTGG | 2676 |
| hs132686777 | GAGGAATCAGAAAACTGAAAAGG | 2677 |
| hs132687639 | GATGATGTATCTTTGATTGAGGG | 2678 |
| hs132689566 | GTTATCATAAATCCTGTCCTAGG | 2679 |
| hs132691068 | TTAGTTTAAAAGAACTCTGTAGG | 2680 |
| hs132683729 | GCTTGATCTTTCATAAACACTGG | 2681 |
| hs132685286 | TCCTGGCTGTTAGACTCACTGGG | 2682 |
| hs132684653 | CTTCAAGGGCACAGATTACAGGG | 2683 |
| hs132683604 | ATTGAAGAAATGTATTAGAGTGG | 2684 |
| hs132694338 | TTACGTATTCCTCTTAGAAAGGG | 2685 |
| hs132684504 | GGAGGAAAGCAGTTTTGCATTGG | 2686 |
| hs132692163 | ATATCTAATGTTTCATCACATGG | 2687 |
| hs132686964 | TGCACCTGTTTCTTCCACTCCGG | 2688 |
| hs132691390 | GATATTTTTTATACTATCAAGGG | 2689 |
| hs132693828 | GAGCCTTTAAAATACCATGTAGG | 2690 |
| hs132684604 | GTTTCCTTTTGTTCTCTGGAGGG | 2691 |
| hs132691585 | TTAGCATTGTTATTCCGTCTGGG | 2692 |
| hs132685106 | ACTTGCTCGATTAGGTTTGGTGG | 2693 |
| hs132684169 | TATTGATGCCCCTCTACAAAAGG | 2694 |
| hs132693232 | CCCAATTGAGTGTTTTCCAAAGG | 2695 |
| hs132686824 | TTAGTTTTAATGGCCCAAAAGGG | 2696 |
| hs132683665 | AGGGCTCCTCAATTGTTGCATGG | 2697 |
| hs132687281 | TACTGCAGAACAATTATGGCCGG | 2698 |
| hs132683753 | ATTTATTTCTGTACTAAGATCGG | 2699 |
| hs132684067 | AGCTATTTAGGCTTGGTCGCCGG | 2700 |
| hs132689010 | TCAGGGATTCACAAGAGAGCAGG | 2701 |
| hs132691873 | ACAGACTAGTTCTTAAAGATAGG | 2702 |
| hs132690745 | TTTCTTGTGTAGTAATAACTTGG | 2703 |
| hs132686829 | AATAGACTAAAACTTTACATAGG | 2704 |
| hs132686578 | ATCCTTTGTCTCATATACTACGG | 2705 |
| hs132688970 | GCAGTTAACATTCATATGAAAGG | 2706 |
| hs132692570 | CTACAGGCCATGGGAATGATGGG | 2707 |
| hs132693366 | AGAGATTAATGGCAAGAAGAGGG | 2708 |
| hs132692965 | AGGAGCTGCAGCTTTTCAAAGGG | 2709 |
| hs132692657 | CATCCCCTCAAATTGTTCAATGG | 2710 |
| hs132688920 | GGAAGTCAACAAATTGTCAATGG | 2711 |
| hs132688506 | TATCAATCATCGCTGAGAAAAGG | 2712 |
| hs132686966 | GCAACCGGAGTGGAAGAAACAGG | 2713 |
| hs132686325 | AAAAAATTATTCCAGTTACATGG | 2714 |
| hs132689585 | ACTTATTTGCGCGGGTAATGAGG | 2715 |
| hs132693747 | AGAATCATTTTCCAAGCAGATGG | 2716 |
| hs132688558 | TTATACTTATATTCAAGCACAGG | 2717 |
| hs132690504 | TATCTCATACACCTTTTGCTGGG | 2718 |
| hs132686591 | TCTAATTTACATCCTATACTTGG | 2719 |
| hs132693674 | TCTGGGTTTTGCATGACAGCTGG | 2720 |
| hs132683412 | GCCGCAGGTATGTCATTCATAGG | 2721 |
| hs132689345 | GATCTGTGAGAGATGGGAATAGG | 2722 |
| hs132685723 | TTAAAAAAAAAGAAGCCTCTCGG | 2723 |
| hs132688034 | GCCTTTGACCATATTCTGAAAGG | 2724 |
| hs132687297 | GATTGCATATCTTTCCCATAGGG | 2725 |
| hs132685700 | TATCTAAATGTGAAGTTGGCGGG | 2726 |
| hs132693510 | CCTGGATTATTTCCATCTATGGG | 2727 |
| hs132685944 | TTCTGAGCTGCCTTAGCAAAGGG | 2728 |
| hs132683693 | AGTTTACATTATTTGCTCATCGG | 2729 |
| hs132688519 | ACAGCATAATTTTCTACATACGG | 2730 |
| hs132686771 | TGTGGCCCAAAAGAAAAGATAGG | 2731 |
| hs132690579 | GCTGCATTAATCATAATGCTTGG | 2732 |
| hs132684505 | TAATTCTGAGGGCGTTTTGGAGG | 2733 |
| hs132687753 | TTTTGTGTTTAGAAATGCTAGGG | 2734 |
| hs132683442 | TTATTTTTATTCAGTATATGTGG | 2735 |
| hs132692154 | ACACCTTTTTAAATACACCTTGG | 2736 |
| hs132687967 | TTTAAGTGACTGTCTATTGGAGG | 2737 |
| hs132690432 | GTGTTGACTCACTCAGTGTTGGG | 2738 |
| hs132684346 | CTTACCTTACTTGCAAAGGATGG | 2739 |
| hs132689185 | ACAAAAAGTTAGTTTTCTGTAGG | 2740 |
| hs132691745 | AAAGACATACTAGGATAAAGTGG | 2741 |
| hs132688578 | TTAACATCAGAAAGAACGTTGGG | 2742 |
| hs132683487 | TTAATGATAAACATTAGATAAGG | 2743 |
| hs132683953 | GATGCATATGAGATTATTGGAGG | 2744 |
| hs132685743 | CAATCTAGAGGAAATGGCTTCGG | 2745 |
| hs132690261 | GGAAATATTTTCATGAAACCTGG | 2746 |
| hs132690328 | ATTTAAGGGGAAAAAGGTGTTGG | 2747 |
| hs132690763 | GTGTCATCCTAAGAAACAATTGG | 2748 |
| hs132693173 | TGCTGCCTGCTGTATCTCACTGG | 2749 |
| hs132691781 | TCACTTGAAGATTTTTAGTAGGG | 2750 |
| hs132686690 | GCAAGATACTGCATGGAACTCGG | 2751 |
| hs132686371 | TTCTTCAAGGATGTGTTGTAGGG | 2752 |
| hs132688862 | TGACAAGTGATCCTCAAAACAGG | 2753 |
| hs132687084 | ATAACAAATTAAGATCTAAAGGG | 2754 |
| hs132683913 | GGAATTCACAAACACAGATAGGG | 2755 |
| hs132685061 | CATATGTTCTGTACATCAGCCGG | 2756 |
| hs132689120 | AAGCTGGTAAACCTCATTGTAGG | 2757 |
| hs132690842 | CATTGTGGATGTAGAGTCATTGG | 2758 |
| hs132690051 | GGGTGACCACAGTTTAAAGAAGG | 2759 |
| hs132692400 | ATATGCATACCTCTTAAAATGGG | 2760 |
| hs132688463 | CATAGGATCACAATTCATCTAGG | 2761 |
| hs132688161 | ATCCCTCACGTTCTTCAGGTTGG | 2762 |
| hs132692935 | ATTTAATTAATCTTCAGTGATGG | 2763 |
| hs132685333 | TTAAAAAAATACAATGGTAAGGG | 2764 |
| hs132694315 | CTACAGGGTTATTTTGTGGTTGG | 2765 |
| hs132683567 | TATGTCTAATGGGTGCTCAATGG | 2766 |
| hs132688138 | TGGATGTAGTAGTTGAGTAGTGG | 2767 |
| hs132689494 | TTATGCTCATCTCTAGGTATGGG | 2768 |
| hs132690126 | AATAATATGGGACACCGTAATGG | 2769 |
| hs132691750 | TGTAAAATGATGAAAGCAAAGGG | 2770 |
| hs132691651 | GCTCTTTTGGTGTGATCAAGGGG | 2771 |
| hs132683425 | GCTGCTTGAAATGACTAAACAGG | 2772 |
| hs132685897 | GCAAAGCTTGAAATAGCAAAAGG | 2773 |
| hs132686178 | GTAAAATATGACAAAACTCTAGG | 2774 |
| hs132692980 | GATACAGTCTAGGGGTGGACAGG | 2775 |
| hs132687687 | CAGCCCTCTACCCTAAATAAGGG | 2776 |
| hs132691410 | GTAAACTATTGTAGCTCTAAGGG | 2777 |
| hs132688923 | GAAACTTGATAGCCTTTTGGAGG | 2778 |
| hs132688405 | ATTAAGCTCTACATTGGCTAAGG | 2779 |
| hs132691694 | AATCTGAAGGACAAGAAGCTTGG | 2780 |
| hs132684523 | TCTGAAACACTGAAGATCAATGG | 2781 |
| hs132683490 | TTTTGGAGCATTATAAAGGAAGG | 2782 |
| hs132687597 | ATACATAGCAAGACATGTATTGG | 2783 |
| hs132687721 | GTGTATGCAACTAAGATGATTGG | 2784 |
| hs132687194 | GGGATGGCTGGGGGGAATGTGGG | 2785 |
| hs132693526 | GTAGTGTTTGCAGCATGAAAAGG | 2786 |
| hs132687392 | TAGAAAGGATATTTCTAGAGAGG | 2787 |
| hs132684879 | AATTTTTGAGAATGGAAGCTGGG | 2788 |
| hs132686422 | ATTACTTTCGATCTCAAGACTGG | 2789 |
| hs132692476 | TGGCTAAAACATCTATAGAAAGG | 2790 |
| hs132691768 | CATTCTGCATATATTTAACATGG | 2791 |
| hs132687844 | GCTCCATTTAAAACTATGGCAGG | 2792 |
| hs132693937 | TGATGCCGAAAAGAACATATAGG | 2793 |
| hs132692295 | GAGAATATTGAGAAATAAGGTGG | 2794 |
| hs132688962 | GTATTACCAGCCAAGTGCCTTGG | 2795 |
| hs132692176 | TTGTGTTCTAAATTCAAAGAAGG | 2796 |
| hs132688631 | GGAGGCCCCAATACTGTTGAAGG | 2797 |
| hs132694321 | GTATAGTGTGTTACAGAACTTGG | 2798 |
| hs132685965 | ATTGTGAAACAATCTCAAATAGG | 2799 |
| hs132689351 | ATAGCAGTCAATGCTTATATGGG | 2800 |
| hs132692348 | CTAATTTGTTGCCCATGTCAGGG | 2801 |
| hs132687596 | CTTGCTATGTATGGCCAAACGGG | 2802 |
| hs132690782 | ATTTGATTCGAATTTAAATGAGG | 2803 |
| hs132691327 | AGGAAAATTGCAGAATGCAGAGG | 2804 |
| hs132693333 | GCTCCATGCATTTTTAAGTACGG | 2805 |
| hs132692424 | GGCTTTAAAGATCTAAACTAAGG | 2806 |
| hs132690539 | GATTATCAACTAAGGCTGAATGG | 2807 |
| hs132694143 | GTAAAACTTTACTTGTATGTTGG | 2808 |
| hs132691479 | AAGAGAGTATCCATCTCCTAAGG | 2809 |
| hs132683524 | GGATATGGGTGACTGCCAACAGG | 2810 |
| hs132692726 | TTGTTTGCTTGATTTATACATGG | 2811 |
| hs132693226 | AAATAACTCTTTAAAGGAAAGGG | 2812 |
| hs132684801 | ATAATTGAGTTAGAAGGTAGAGG | 2813 |
| hs132687581 | TGCCAGCGTGATTTCTTCAAAGG | 2814 |
| hs132690821 | GAGTACAATTGGCTCCTAGAAGG | 2815 |
| hs132688009 | CTAGCTCCCTGCCACAAAGATGG | 2816 |
| hs132693922 | TTAGTTCTTCTGCCATACATAGG | 2817 |
| hs132683609 | GTTGGAAAAACAGTGCCATCTGG | 2818 |
| hs132689674 | TTCCAAATTTAAATTCCCATTGG | 2819 |
| hs132693943 | AACTCCTTGTCTCTTTTGGAAGG | 2820 |
| hs132685876 | ATCCTTCTATGAGCAAAAGTTGG | 2821 |
| hs132686836 | ATTTTACTGCCAGTTTCAGCAGG | 2822 |
| hs132691620 | TATTCAACAGTCACAGAAAGAGG | 2823 |
| hs132690599 | ATAGAATTCAAGCAATTTGGAGG | 2824 |
| hs132690596 | AAACTAGCATATTTTCCCATTGG | 2825 |
| hs132691432 | AGGTGGCTGTTAATGATAAGAGG | 2826 |
| hs132690628 | GTTGGAATAGCAATAGAAAAGGG | 2827 |
| hs132693688 | AATTTTATTTTCTAACCTGTTGG | 2828 |
| hs132688455 | TGTAATTTAGGTGAAATTGTTGG | 2829 |
| hs132692362 | CTGTTTGTTTTCTAAGCAAAGGG | 2830 |
| hs132687339 | GCCTTTGTTGTTGCCTTCAGGGG | 2831 |
| hs132693310 | ATATTTTTATTACAAGTATGTGG | 2832 |
| hs132691155 | GGAAAATCAAACCAAGCAAAAGG | 2833 |
| hs132686079 | AGCTGATGAGATTTTAGAAGAGG | 2834 |
| hs132689244 | GCACAGAAATGTAGATTTCGCGG | 2835 |
| hs132690234 | TTCTGATAACCCCTTCGAATGGG | 2836 |
| hs132688666 | CTTGATGAATTCAGAATCTAGGG | 2837 |
| hs132686421 | CCTTGAAATTGCTAATACAATGG | 2838 |
| hs132684137 | TCTTATCTATATGATGGGATGGG | 2839 |
| hs132692853 | AGAGACTAACCAGTGACAACTGG | 2840 |
| hs132687263 | TTCTTTTTGTAATAGGAGATGGG | 2841 |
| hs132687586 | GACATATTCCTGACTCCAGCAGG | 2842 |
| hs132691896 | ATATAATTCTTATTATTTCAGGG | 2843 |
| hs132684232 | CCACAGTGAAAGAAACAAACAGG | 2844 |
| hs132687649 | CCATTAACCTTATCTTTGAAAGG | 2845 |
| hs132684605 | AGTTTCCTTTTGTTCTCTGGAGG | 2846 |
| hs132689331 | GTAAGTTGTTGCTAAATTGTGGG | 2847 |
| hs132683397 | TTATGGGGAATTTAAAAGTGTGG | 2848 |
| hs132683994 | AACCATGATATCTCCAACAACGG | 2849 |
| hs132694073 | TTTTGAGCAAACGTTTATAGAGG | 2850 |
| hs132683792 | ACAGTAATTGGCACTGAAAGAGG | 2851 |
| hs132689799 | TAAATCTTAGACTAAAATGTTGG | 2852 |
| hs132692040 | ATTCCAGATAAGAAGACCCTTGG | 2853 |
| hs132686601 | CTTGCTTTACTCTTAAGCACTGG | 2854 |
| hs132688955 | TATATTATGCCCAAGGCACTTGG | 2855 |
| hs132693950 | TTACTTTCTTTAATCTACAAAGG | 2856 |
| hs132683634 | AGGTCTCCAGTGTAATTTAGAGG | 2857 |
| hs132687214 | TTCAAAGAGTACCATATAAGCGG | 2858 |
| hs132691375 | ACATAGACAAAGAAATAAGTAGG | 2859 |
| hs132690347 | GCAGCGTGAAAAAATCCTACAGG | 2860 |
| hs132694322 | CAGTACTTAAAAGTATTAAACGG | 2861 |
| hs132694083 | ATATTGCAATTAGTATCTATAGG | 2862 |
| hs132691728 | GTTTCCAAACCGGAACACTTTGG | 2863 |
| hs132685355 | TTCTTAGCAGAAAAACTCATAGG | 2864 |
| hs132684888 | TCAATACTATTAACAACCCCAGG | 2865 |
| hs132691017 | TCCTCACTATGAATTAGATTAGG | 2866 |
| hs132684291 | ATATTCTGCACTCCATCGTCAGG | 2867 |
| hs132690722 | CCAAGGTATAATCTATTTGAGGG | 2868 |
| hs132686758 | AGAGAAGTGGAAGGTTTAGAGGG | 2869 |
| hs132685792 | CTTCAAGCCCCCAGTGAAGTAGG | 2870 |
| hs132689349 | CTTGTTTCACAACATTTATGTGG | 2871 |
| hs132690926 | CAAAAAATCGACTCTTGGAATGG | 2872 |
| hs132693618 | TCTGACAAGTTGGCTTAAAAGGG | 2873 |
| hs132688696 | CTTTCCCTCACAGATCCCTCAGG | 2874 |
| hs132687768 | GCATCCAAATACCCCTCATAGGG | 2875 |
| hs132689808 | ATGTATCTGTTTATACAAGTTGG | 2876 |
| hs132683887 | TTTTCCATCTCTCATTCAGATGG | 2877 |
| hs132691064 | CTTTTGTTGTGCACGTATATGGG | 2878 |
| hs132689026 | GGGCATACATCCGTGTTACTAGG | 2879 |
| hs132685001 | ATAAGGGCAAATTAAAATGCAGG | 2880 |
| hs132688147 | CTGGGCAGAGAAGTCATGCTTGG | 2881 |
| hs132686143 | ACAGTGTTACATTTAATGCTAGG | 2882 |
| hs132688316 | AGCTTATGATCAGATGGTTAGGG | 2883 |
| hs132693890 | TATCTACTCTGCATGAAGCTAGG | 2884 |
| hs132687894 | AATAGACTATTTTGAAGCAAAGG | 2885 |
| hs132683815 | AGTACATTACAGAAAAAGGCTGG | 2886 |
| hs132692671 | TAGGCACTGTGCAAGTTAGTTGG | 2887 |
| hs132685067 | GTGTGTGTGTGTATTGTAGCAGG | 2888 |
| hs132691811 | ATTGTAAAGTCAATAAATAACGG | 2889 |
| hs132685785 | TTATCAAGATACTTGAAAGCAGG | 2890 |
| hs132684669 | AATTCTGCTCTTGAGAAAAGAGG | 2891 |
| hs132685572 | TTTGTCACAACCCTAGAAATGGG | 2892 |
| hs132689033 | GGTGTAACAAAAAGTGATAATGG | 2893 |
| hs132684489 | CCATCATTTGCAGGGCTGGAGGG | 2894 |
| hs132694181 | ATGGAGATTGTAATATTTGGTGG | 2895 |
| hs132691812 | TTGTAAAGTCAATAAATAACGGG | 2896 |
| hs132687684 | ATTCCCTTATTTAGGGTAGAGGG | 2897 |
| hs132689825 | TTACTTGCCTTTGTAGGTAGAGG | 2898 |
| hs132683402 | AGTCTCTAATAGCAAACAAAAGG | 2899 |
| hs132689053 | AGGTCAGTCACTGGACCCTCAGG | 2900 |
| hs132691948 | CTGACTTTATTTTGTCCCTAGGG | 2901 |
| hs132687771 | GGCTTAGTCCTCAATAAGTAAGG | 2902 |
| hs132693705 | GCTGTGTCTATGGGGTTATAGGG | 2903 |
| hs132684969 | TGTGTGGGAAACCCTGAGAATGG | 2904 |
| hs132692005 | GGTTTCCAACAAGAGCCCTCCGG | 2905 |
| hs132688288 | TCTACTCTTGGGCTTATTCGTGG | 2906 |
| hs132694267 | AGAAGGTGTGAAAGAGAAGTGGG | 2907 |
| hs132692537 | GTAAGGATTTTGAAACTTGGAGG | 2908 |
| hs132688804 | AATTCATGATTTTAGTTGGAGGG | 2909 |
| hs132690157 | CAGTTCCCATTAGTGAGGAATGG | 2910 |
| hs132692545 | TTATGGATTGTTTGACTGGAGGG | 2911 |
| hs132685656 | CCTGTCTCCTAAATTTTGCATGG | 2912 |
| hs132692909 | ACAAAAGCTTACATTCACTTTGG | 2913 |
| hs132691901 | ACAAGTCTACTGGAAGACTTTGG | 2914 |
| hs132684104 | TCATCATCCGGGATTCTTGTAGG | 2915 |
| hs132686249 | TTTTTTTTGTGTGTGTTGGGGGG | 2916 |
| hs132692647 | CAGCCAGCTTTCCAGAAGGCTGG | 2917 |
| hs132686810 | ATGTTCGTAACTGAGAGTTAGGG | 2918 |
| hs132692781 | TGTACATAAAAAACTGGCCTAGG | 2919 |
| hs132693059 | TGTCAGGGTCCTGGTGATAATGG | 2920 |
| hs132685030 | AGAAACAGACAAGATTACTTTGG | 2921 |
| hs132685047 | TATAATTTTTCCTAGAATGAGGG | 2922 |
| hs132690594 | TCTCTTTAGTTAAGTCCAATGGG | 2923 |
| hs132688916 | TACGTTATTTAGCTCCCTTAGGG | 2924 |
| hs132685472 | TTTTTACCTGAGTTGAGAAAAGG | 2925 |
| hs132693663 | ATTCATAAGAAATTTTTCAATGG | 2926 |
| hs132690104 | GAGCTAAGTGCTGACTTAAAGGG | 2927 |
| hs132689588 | TTTTCATTCCTAATGCCCTAAGG | 2928 |
| hs132690237 | CACTTTTAGTATTAAGTAGAGGG | 2929 |
| hs132687243 | TATTGTAGCCAGATCTTTGGTGG | 2930 |
| hs132684617 | TTAAAACGTGTATTTAAAAGTGG | 2931 |
| hs132687921 | TGCCTTATACTACCTGACTCTGG | 2932 |
| hs132685415 | CCTAAGCATCTTTTTTCAAGCGG | 2933 |
| hs132684707 | CAGAATCTTCAACATACAAATGG | 2934 |
| hs132694248 | ATATAATCCATCTTTTAAGAAGG | 2935 |
| hs132693611 | AGATGACAAGTGAATCTCATAGG | 2936 |
| hs132687417 | GATTTCCAACCACTTTAACTTGG | 2937 |
| hs132687049 | TAGCAGTGCTACTAAGTGAATGG | 2938 |
| hs132684540 | TCATATGGCAAACTAAAGAAAGG | 2939 |
| hs132689453 | GTGGGAAGTACAATAGCAACTGG | 2940 |
| hs132693779 | GTATACAATTGGATTCTGTCAGG | 2941 |
| hs132685150 | TCTTTCACTTAAAATAGCTCTGG | 2942 |
| hs132684541 | CATATGGCAAACTAAAGAAAGGG | 2943 |
| hs132683819 | TTAAAATATCTTAAGCAGTCAGG | 2944 |
| hs132686595 | TGATATCCTCAGCCAAGTATAGG | 2945 |
| hs132686610 | TGCTCCCCTCAAAAGTCCCTTGG | 2946 |
| hs132690663 | GTATCCATCCAAGACTGAAATGG | 2947 |
| hs132691870 | AGTTTTTCCCTTTCACTCATCGG | 2948 |
| hs132687877 | TGAAGAATAAACAGGCCTTTGGG | 2949 |
| hs132686842 | GGGAAGATCAAAGAAAAATCAGG | 2950 |
| hs132684293 | TCGGGCATAAAGCATTTCCTTGG | 2951 |
| hs132691275 | TCTTACTTAAAGGACTGATTCGG | 2952 |
| hs132685270 | TCAGCACTAGCAGCACTTGTTGG | 2953 |
| hs132687146 | ATTACTTTGTCAGTACTTCAGGG | 2954 |
| hs132684833 | TTAAAGCCACATCCACTGATAGG | 2955 |
| hs132691012 | TGCTAATTAATGGGGACCCATGG | 2956 |
| hs132685201 | GCACACTAAAAGGCCTATAGAGG | 2957 |
| hs132686866 | TGAGAGAGAAGACTTGCAAAAGG | 2958 |
| hs132683687 | ATTATTTCCTCTTTTCAGACTGG | 2959 |
| hs132693684 | TGCCAGTTATGGGCTTAAGATGG | 2960 |
| hs132684736 | ACGTACAATGTCGTTCTTGAGGG | 2961 |
| hs132694063 | TTATGTTGCAATTTGAAGACAGG | 2962 |
| hs132691969 | TTACAGTCATAGTAGGTTGTGGG | 2963 |
| hs132684039 | CATGGGACCCGCCTGTTCAGAGG | 2964 |
| hs132692015 | ATAAATTTACAAGGGGCCTATGG | 2965 |
| hs132684290 | CACTCCATCGTCAGGTAAGAAGG | 2966 |
| hs132686847 | AATCAGGTCATGGGGCTTTGCGG | 2967 |
| hs132683646 | ATACTTCTTTGCAAACCAAAAGG | 2968 |
| hs132692327 | GCCTCCTCCAGGAAGATTTGAGG | 2969 |
| hs132691704 | CTCCAGCTGCATTTATATAGAGG | 2970 |
| hs132685036 | ATTTGAGGATGATATTCAACAGG | 2971 |
| hs132685582 | ACTAGCATCTGACAGCCTATTGG | 2972 |
| hs132687442 | GTTATTCATTCAGCAAAAGAAGG | 2973 |
| hs132694292 | TATTTGGTCCCACTGATGAATGG | 2974 |
| hs132691900 | TCTAGTCTAAACAAGTCTACTGG | 2975 |
| hs132693945 | GCAACACATACAATAAATGTAGG | 2976 |
| hs132686487 | GATCATTCATCCCTAGGAACAGG | 2977 |
| hs132684518 | CATTTTGATGAAGCGATGTAGGG | 2978 |
| hs132685793 | TACTTCACTGGGGGCTTGAAGGG | 2979 |
| hs132687204 | GTATGACTCTGCTTTATTGGAGG | 2980 |
| hs132688477 | AAACACACAGGACAAGAAGATGG | 2981 |
| hs132688663 | TATTTACCAATTACTGACATAGG | 2982 |
| hs132684847 | CTACGCATGCATAGAAAACTAGG | 2983 |
| hs132692977 | TGCAAGAAAGATACAGTCTAGGG | 2984 |
| hs132689649 | ATTAGGGTATATTTCCCCTCTGG | 2985 |
| hs132687091 | CTGATCATTTATCCTTTTAGCGG | 2986 |
| hs132689933 | TATTCTCTCATCTCATAAAGAGG | 2987 |
| hs132691301 | TCACTTGTATTTATACAGAATGG | 2988 |
| hs132687460 | CCAGGAACATGCTATATAGCTGG | 2989 |
| hs132692691 | CTTTATTGTAGCATGACTTAGGG | 2990 |
| hs132685098 | TCAGTTCTTACTTCGTAGAATGG | 2991 |
| hs132690345 | ATGAGTTTGGTAAGAAAAAACGG | 2992 |
| hs132688208 | CCTGCTCCATAACAGGCAATAGG | 2993 |
| hs132693519 | AAATGAACAGACTTTGGTACTGG | 2994 |
| hs132686006 | TGAATAATATTGGTTAATATGGG | 2995 |
| hs132684299 | AGCTCTGGATTGTTTAAGGATGG | 2996 |
| hs132689524 | TCACTTAATTAATTAAAGCCTGG | 2997 |
| hs132692369 | ATAATTGCTGACATTTAAAACGG | 2998 |
| hs132691269 | AGTCCAGTTAAAGGTAGCTTGGG | 2999 |
| hs132690203 | TTCTTAAGCACAATTCTGATAGG | 3000 |
| hs132692249 | ACATTTAAAATGAAAACAATGGG | 3001 |
| hs132683431 | TTTACTTGAAAACTGTTCCCTGG | 3002 |
| hs132692520 | AATAAGTGATAAATAACAACAGG | 3003 |
| hs132693665 | TTCAATGGCAAAATGAGGCTTGG | 3004 |
| hs132685008 | CTTTGGCAATAGGATTCAAATGG | 3005 |
| hs132687232 | ACCATAATGTTGTTGAAGATGGG | 3006 |
| hs132684868 | GGGCACAGCTACCAGGAAAAGGG | 3007 |
| hs132688169 | GTGTGGCTCTGGTTTTCGCCAGG | 3008 |
| hs132685087 | TTTCTTTTTCTTACTAGTAGAGG | 3009 |
| hs132693554 | TAACAGTATTAAGATCATAAGGG | 3010 |
| hs132689032 | CATTTTTTAATACTCAAAATGGG | 3011 |
| hs132694185 | CTTCTCATATCCCTATGTTCAGG | 3012 |
| hs132691859 | CTGGAGCATTTAGAGTAAAAGGG | 3013 |
| hs132686969 | TGAGAACACCTCACTATTCACGG | 3014 |
| hs132687390 | AACCTCATCATATATAATGATGG | 3015 |
| hs132693697 | TCAGCTAGAAAGCAGCTAATGGG | 3016 |
| hs132683715 | TTTCTGTCTGTGCTAATACTTGG | 3017 |
| hs132687999 | ACTAGCCAGAAGGAGCAGTATGG | 3018 |
| hs132694340 | TTTTTCTGAATGTTAGTCCATGG | 3019 |
| hs132683581 | GCAAACGCCACTTCTTTCTCAGG | 3020 |
| hs132684047 | TCTGAACAGGCGGGTCCCATGGG | 3021 |
| hs132691277 | TTGGTTAGTTTATCAACCCCTGG | 3022 |
| hs132691303 | TGAGCATGAGTCTTACAGTATGG | 3023 |
| hs132691731 | AGTAGCAGAACTTTTGTTGGTGG | 3024 |
| hs132691726 | ATAACTATACCAAAGTGTTCCGG | 3025 |
| hs132692845 | AGACTAGCTCTTATTACCCTTGG | 3026 |
| hs132685230 | GTAAATTATTTTCATACAAGTGG | 3027 |
| hs132689326 | AATCCCCCAAGAGTGCTCTATGG | 3028 |
| hs132692944 | CTGTGCCTATCCTAATAAGCAGG | 3029 |
| hs132694268 | TAGAAGGTGTGAAAGAGAAGTGG | 3030 |
| hs132693373 | CTTACCACAGATATATTCCTAGG | 3031 |
| hs132691997 | GCCTAGATCCTGACTGTCACAGG | 3032 |
| hs132683326 | TCTGACTTTTAGATGAAAAGAGG | 3033 |
| hs132692257 | TGATATTTGAAGTCAAGAACCGG | 3034 |
| hs132690841 | TAAGTATCTTAAAACCCTAAAGG | 3035 |
| hs132687916 | TTTCAGACAAGAAAAGCTTTGGG | 3036 |
| hs132685423 | GCAAATCTATCTAGAAAAAAGGG | 3037 |
| hs132690603 | AATTTTAAAGGGAATTAAGATGG | 3038 |
| hs132690674 | GCATGTGTGATAAATAAATTGGG | 3039 |
| hs132687289 | ACTCTTTGCAGCTCTTCAGCTGG | 3040 |
| hs132686294 | TTTGTACTATCAGCTATAGATGG | 3041 |
| hs132690499 | GAGTCTGTCACTTTAACAAATGG | 3042 |
| hs132691244 | TGATGATGCTGAGGACTAGATGG | 3043 |
| hs132692663 | GGAGGCCATTGAACAATTTGAGG | 3044 |
| hs132683637 | GTAGGCAGCTGTATGGAAACAGG | 3045 |
| hs132688402 | TTAATTGTAATCCTTAGCCTTGG | 3046 |
| hs132684999 | AAGAATCCTTCCTTTCATAAGGG | 3047 |
| hs132692244 | ATGTTGTCCTTTTTCTTGCATGG | 3048 |
| hs132694138 | TCTTGCCTGAAATTGGTAAGAGG | 3049 |
| hs132683884 | TGAACTATCTTTATCAAATGTGG | 3050 |
| hs132686078 | CTAAAATCTCATCAGCTCCTAGG | 3051 |
| hs132685395 | GAGATTTTAACATATTAGAATGG | 3052 |
| hs132694198 | GCTACCCTCTTCTCTCTTCGTGG | 3053 |
| hs132690634 | CTTGCCCTCTGATGTCCAGCTGG | 3054 |
| hs132689621 | TTAACGATGACTGAATTGATAGG | 3055 |
| hs132684937 | GTAACTCCTGGTACACTTGTAGG | 3056 |
| hs132693748 | GATGCTGGCTTCCATCCTACTGG | 3057 |
| hs132690056 | TTAAATGTCTGAAAATGGATTGG | 3058 |
| hs132685325 | AAGAAGCTATAGAAATAGATTGG | 3059 |
| hs132689894 | TATCTCTTATATGCATCAGTTGG | 3060 |
| hs132688035 | ATCCAATTGTCATTTCTAAGAGG | 3061 |
| hs132686466 | TACCAGATTGAGGATATTAGAGG | 3062 |
| hs132690502 | GGTGTATGAGATAAAACATAAGG | 3063 |
| hs132692761 | AAAGGTGATGTAGGTGTGAAGGG | 3064 |
| hs132693683 | GCCCATAACTGGCAACCAACAGG | 3065 |
| hs132689024 | GGATGTATGCCCTCAGAGAATGG | 3066 |
| hs132692437 | ATAAGGACGTGTCTGCTTGTGGG | 3067 |
| hs132692436 | TCCTTATGAGATGCATGCCCTGG | 3068 |
| hs132690129 | GCTGAGCACAATTACCATTACGG | 3069 |
| hs132684602 | TTTTGTTCTCTGGAGGGAGGTGG | 3070 |
| hs132685108 | CGATTAGGTTTGGTGGTTGGAGG | 3071 |
| hs132691367 | GTCAGCCAGAAAATATATATAGG | 3072 |
| hs132684802 | GGTCAGATAATTGAGTTAGAAGG | 3073 |
| hs132689253 | TTTTTTTCCTTGTTGAGTAAAGG | 3074 |
| hs132684595 | CAGTCTATGCAGAGATTCAATGG | 3075 |
| hs132683512 | CACTCTCTTGGAAATGAGAAAGG | 3076 |
| hs132683800 | TGGCCTAGGGAATAAATAATGGG | 3077 |
| hs132691755 | TCTGCCGCTGATGGTTTGTTCGG | 3078 |
| hs132684793 | CTCTTCCTCTTTTCTCACATGGG | 3079 |
| hs132691153 | TTTCAAATGATCCTTTTGCTTGG | 3080 |
| hs132687318 | TTTCTCAATGAACTGAGTAATGG | 3081 |
| hs132690719 | CCTCAAATAGATTATACCTTGGG | 3082 |
| hs132684859 | ATATGAGAGTCTCTGAATAAGGG | 3083 |
| hs132689727 | TAATACTTTGGAAATTGAAAGGG | 3084 |
| hs132684598 | ACCTCCCTCCAGAGAACAAAAGG | 3085 |
| hs132688609 | AGTGTGGCTGGGAGTCTACCTGG | 3086 |
| hs132693009 | TGTTTCCACTGGGAATATATAGG | 3087 |
| hs132692401 | GTATGCATATGTACATAAATCGG | 3088 |
| hs132683982 | GATTGACACCCGTAGCCTATGGG | 3089 |
| hs132693303 | CCAGTAGGTGGGTCACTCATTGG | 3090 |
| hs132690099 | ACAAGTGCAGATTTTAGCCTTGG | 3091 |
| hs132692080 | TTTTTTCATATATCATCAATGGG | 3092 |
| hs132689920 | GATTATCATGTGCGTTTCCATGG | 3093 |
| hs132685376 | AAGCTGAAAGGTAAACAAATCGG | 3094 |
| hs132685789 | ATAGAACTTTCTATGTATAGAGG | 3095 |
| hs132693368 | CCAGAGAAAATGAAGCTAAAGGG | 3096 |
| hs132694260 | TATCACAAAAAGAGAAATAAGGG | 3097 |
| hs132684931 | CATAGTATGCAGATTTTAAGTGG | 3098 |
| hs132685055 | AATTCCCTGGTTTTGCTCCCCGG | 3099 |
| hs132688408 | GTTTATTATTGATAATCTAAAGG | 3100 |
| hs132686605 | AGATGAACGTTTCTTTTCCTAGG | 3101 |
| hs132684850 | TGTAACCACTTACACACTAATGG | 3102 |
| hs132692588 | GTACAGTGTTCTTTATCGTATGG | 3103 |
| hs132686480 | GTTCCCAGATTCCTGTTCCTAGG | 3104 |
| hs132686855 | GCTGTGAAGGTTCAATAAATAGG | 3105 |
| hs132689846 | ACGTTCCCATATAATAGAAATGG | 3106 |
| hs132688189 | CCACTGGGCATGCTCAGAATAGG | 3107 |
| hs132693920 | TCTGCCATACATAGGTCTTAGGG | 3108 |
| hs132694087 | AGTGTCTCCTTTGATAGTGTTGG | 3109 |
| hs132691761 | AATAGTTAAGATGTTGTATGAGG | 3110 |
| hs132683544 | ATTGATTGATAAGTTATGCCAGG | 3111 |
| hs132687540 | GGAGGTTGACAAGGGAAAAAGGG | 3112 |
| hs132694336 | TTTAACTCACCCTTTCTAAGAGG | 3113 |
| hs132684779 | CTACAGCAAGATGTTCCCTTAGG | 3114 |
| hs132691090 | GCATGTCATATACCTGGTTATGG | 3115 |
| hs132689763 | CCTGCTTGAGCTATTGTTGGAGG | 3116 |
| hs132693480 | TTAATATTGTCAAAAAAGCTTGG | 3117 |
| hs132684496 | CTACTTTCATATTTTTAGGTTGG | 3118 |
| hs132691094 | AAGACCTCCCTCTCTTAGCTGGG | 3119 |
| hs132690819 | GGGAAATCTGAGAGTACAATTGG | 3120 |
| hs132686111 | AGTTAACCTTTCCATCCTCTTGG | 3121 |
| hs132691644 | ACGATATGTGCAAAAATTCAGGG | 3122 |
| hs132687870 | TCTAGTTAAGGTTTTTCAGCAGG | 3123 |
| hs132687595 | TCTTGCTATGTATGGCCAAACGG | 3124 |
| hs132685358 | AGGTAGCTCACAGAATAATCAGG | 3125 |
| hs132691281 | TTACATTGTAATGCTTGTAATGG | 3126 |
| hs132692029 | TTTTCTATCACAGATTTACATGG | 3127 |
| hs132692971 | GAAGGCCTAGTGAGGAAAGATGG | 3128 |
| hs132691677 | TGACAGAAACGCTCTTTCAATGG | 3129 |
| hs132689006 | ATAAAATGAAATAAAAGCTTCGG | 3130 |
| hs132691079 | CTATCATTTGCAAAGCACTATGG | 3131 |
| hs132689157 | GAATACACATTAGAAACAATTGG | 3132 |
| hs132692248 | TGTAAATCCTCTCATTAAGATGG | 3133 |
| hs132694309 | TCTTGAATGGTCACTTAAAACGG | 3134 |
| hs132689726 | GTAATACTTTGGAAATTGAAAGG | 3135 |
| hs132693042 | TAGATTATAGAAGTTTACATTGG | 3136 |
| hs132693868 | CACTAGCTACACTATTTTGGAGG | 3137 |
| hs132683736 | AGAGTCAGTCTTTTAGAAAAGGG | 3138 |
| hs132691142 | AGACAGCTGATTATATAAGTTGG | 3139 |
| hs132689884 | ATTAGTCACGTACATACATATGG | 3140 |
| hs132687598 | AGTGCACAAGTCAACCCGTTTGG | 3141 |
| hs132690169 | GAGTGAAAGTGAGGCATTGAGGG | 3142 |
| hs132692957 | TTTTTTTTCCTGAGAGTAAGTGG | 3143 |
| hs132686821 | GTGGCTTTGGTAAGGTAAGTTGG | 3144 |
| hs132685165 | GATATTTGAGGCAAATCCTTCGG | 3145 |
| hs132683623 | CAGCCATGTTAATAATTAGTAGG | 3146 |
| hs132683836 | CAATAATGTTCCTCTTTTAGCGG | 3147 |
| hs132683776 | CCTACTAAACGAATTATAAAGGG | 3148 |
| hs132685550 | AAATCATCAGTAATACATATAGG | 3149 |
| hs132693265 | GTAAACATTTTATAGACAAAAGG | 3150 |
| hs132689749 | ATCATTGTGGCCAGCTCCCAGGG | 3151 |
| hs132693951 | TACTTTCTTTAATCTACAAAGGG | 3152 |
| hs132688643 | TGATAAGGTTAACAATCCTTGGG | 3153 |
| hs132694002 | TTTGAATTACTATTTTTCACTGG | 3154 |
| hs132694294 | GTTTAGGCTTGCTTCATTTGCGG | 3155 |
| hs132690140 | TTCATTACATGGATAAGCATTGG | 3156 |
| hs132691577 | TGTTTATTTGGAATTCCCCTTGG | 3157 |
| hs132690477 | GCTTCTCTTATTCAGACTGTTGG | 3158 |
| hs132687250 | AGGTTTGTACTACTACATATAGG | 3159 |
| hs132691790 | AAGCCTATCAAAAGTGAAATGGG | 3160 |
| hs132685138 | AGGAATTCCAAGCATATAATGGG | 3161 |
| hs132693933 | ATTAGAACCAAATTTGGAATGGG | 3162 |
| hs132688400 | GATAGCTTAAAGCTCAGAAATGG | 3163 |
| hs132686851 | CTAAATGCATGTACAAACTAAGG | 3164 |
| hs132693435 | AGTATAGTGCTTTATAAAAAGGG | 3165 |
| hs132694381 | GTAAGACAAAACTCATTAAAGGG | 3166 |
| hs132690680 | GATGGAGCTTGCCTTCCATCAGG | 3167 |
| hs132691968 | TTTACAGTCATAGTAGGTTGTGG | 3168 |
| hs132689118 | GTTATAACACAGGTAACTATAGG | 3169 |
| hs132694303 | CCATAGTTACTTTTAAAAGAAGG | 3170 |
| hs132688841 | TTATTGTATCTATATAAGATTGG | 3171 |
| hs132686046 | ATAAAGAATGAACTATCTATAGG | 3172 |
| hs132691443 | GTGGCATTCATGTTAGCCTTAGG | 3173 |
| hs132688467 | CCAAATAAGTAGTAAACAAAGGG | 3174 |
| hs132691732 | TGTAGTAGCAGAACTTTTGTTGG | 3175 |
| hs132693329 | TATTTTATAAGTTATTTGAATGG | 3176 |
| hs132689046 | GGATAAAAACAGAATGTTGATGG | 3177 |
| hs132683354 | GATTTGAAGGAGGCTAGAATAGG | 3178 |
| hs132685406 | AAAGTTATGGTACAATTCCTAGG | 3179 |
| hs132685393 | TTTCTAGGCAATCTGCTAGCAGG | 3180 |
| hs132686181 | GTCTGTGTTTGAATTAAGAATGG | 3181 |
| hs132687854 | GGACTTTTTCCCTATCAGTCAGG | 3182 |
| hs132684982 | GATTTCGTTGACCCTTAACTAGG | 3183 |
| hs132684776 | TTAATGAATTTTTATCCCCCTGG | 3184 |
| hs132684079 | GCGGAAAGCCGGGGTAACCGTGG | 3185 |
| hs132693449 | CCATAGTGCCTGACAAAACCTGG | 3186 |
| hs132689702 | TTATAAGCATTCTTCTGCCTGGG | 3187 |
| hs132683722 | TTAAAACAGAAATAAAGTTTCGG | 3188 |
| hs132690851 | TATATCTGACCTTTATTGACAGG | 3189 |
| hs132685739 | TGTAGCTATCACTTACTTCGTGG | 3190 |
| hs132693084 | CTGTCTGTTTGGAGTGGGCAGGG | 3191 |
| hs132692461 | TGTGAACTCCTCCACCTTGAGGG | 3192 |
| hs132687601 | TCAGTGATATTCAGTATGATTGG | 3193 |
| hs132692651 | GGAGATACAGAGTTTTATGTTGG | 3194 |
| hs132693909 | ATTCATGTGCAACCTAAATACGG | 3195 |
| hs132692186 | GATAATTACTTCACTCTACATGG | 3196 |
| hs132693434 | ATACTGTTTTTAGTTGCAAATGG | 3197 |
| hs132686653 | TAGCAAAGAGGAGTAGGTATTGG | 3198 |
| hs132690695 | TTAGCTCTCAAAAGCACATTGGG | 3199 |
| hs132691822 | TATTTTCCCCCCATGGGTAGTGG | 3200 |
| hs132692894 | ATAGACTGTAGGAAGTATAATGG | 3201 |
| hs132683656 | ACACAGGGCCTGGATTATAAGGG | 3202 |
| hs132689474 | CACTCTTCCTGTGACCCGACTGG | 3203 |
| hs132693332 | AGCCTATTGTAGCTGCCTAATGG | 3204 |
| hs132683492 | TTTAGGACATATTTAGAAAGTGG | 3205 |
| hs132684081 | CTCCTTCTCGCGGGGCTCGAGGG | 3206 |
| hs132690066 | GTACACACCCCGTCTCTAATGGG | 3207 |
| hs132686259 | AAATATATCTTGACAGCAAATGG | 3208 |
| hs132691729 | TTTACACTATGTTTCCAAACCGG | 3209 |
| hs132686988 | GTGCACTCATTTATGCAAACTGG | 3210 |
| hs132690921 | GGCAGCGGTGGCTAATACTTAGG | 3211 |
| hs132684843 | TTTTTGTAGGAGCTTGGGCTAGG | 3212 |
| hs132683734 | GTCAGTGCTGAAAACATGTTTGG | 3213 |
| hs132688239 | ATAATAGGTTATTGGAAGGTAGG | 3214 |
| hs132684552 | GCAGTGGTGATTCCAATCATTGG | 3215 |
| hs132689011 | GTGCACACTCAAGCTGTTGATGG | 3216 |
| hs132690356 | GCTTCATAAATTTCTTTTGGTGG | 3217 |
| hs132685173 | ACAAGTTACAGAGGGTTAAACGG | 3218 |
| hs132686948 | GGCTGGCTACACACCTTCATAGG | 3219 |
| hs132686021 | GCTACTTAAGAGAAGGGCTTGGG | 3220 |
| hs132687848 | AAGACGGTTGATATTTCTTAGGG | 3221 |
| hs132693816 | GTACATGGATTCAGGCATATTGG | 3222 |
| hs132691970 | GTAGGTTGTGGGAGAGATGCTGG | 3223 |
| hs132693441 | GTACAAACACCATAACCAAATGG | 3224 |
| hs132684238 | GGTACTTGTCAGGTTTATAAGGG | 3225 |
| hs132692868 | GATAAAAGTCAACAGCTGATTGG | 3226 |
| hs132693518 | ACAACAAAATGAACAGACTTTGG | 3227 |
| hs132688610 | GAGTCTACCTGGATTATTGATGG | 3228 |
| hs132684422 | AGATCTTTGCATGCAGGCATTGG | 3229 |
| hs132694034 | TTGGCTGCTGACCCATTCATGGG | 3230 |
| hs132692660 | AATGGCCTCCTATTGACCTTAGG | 3231 |
| hs132685359 | GATTATTCTGTGAGCTACCTTGG | 3232 |
| hs132686583 | AAGACTAGTCAGATCTCTAATGG | 3233 |
| hs132690993 | TCTTTCCTATTGAAGGCTATGGG | 3234 |
| hs132687356 | AATCAAAGGCTGATTTTAAGTGG | 3235 |
| hs132686314 | ATTAGTGTATTTTTTCAGTCCGG | 3236 |
| hs132686389 | ATAGGAGACGTCCAAATGATAGG | 3237 |
| hs132687608 | TACTACCCTAGAGAGCAATTAGG | 3238 |
| hs132692636 | TTAGTTGTTAATTTAAGTAATGG | 3239 |
| hs132684528 | AGGCAAACGGAAACTGCATTTGG | 3240 |
| hs132683447 | TTTATATTCCTTAGTCCCTCAGG | 3241 |
| hs132690725 | ATTGAAGTTTATCAATTAGCAGG | 3242 |
| hs132686941 | GAATTTATCATTCTTCTGAATGG | 3243 |
| hs132685608 | CTAAATTATCTGATTTTCATGGG | 3244 |
| hs132684197 | TGTTGTGGTTCTGGGTACTAAGG | 3245 |
| hs132688677 | GAGAGCTTCTTATTGAGAAAGGG | 3246 |
| hs132691374 | ATTATAGTACGCACTAACTTAGG | 3247 |
| hs132691088 | ATGACATGCAGCATTTTAAAGGG | 3248 |
| hs132687485 | TTTCTTGCTGGCTTCTGGCGTGG | 3249 |
| hs132692209 | CATTTTGATGTCATTACCATAGG | 3250 |
| hs132688660 | GGCTTGAATGTGACATTGACTGG | 3251 |
| hs132687958 | CTTGTCTGTGTTACAGCAATGGG | 3252 |
| hs132691882 | AGTTTCTTAGTTTTGAGCTATGG | 3253 |
| hs132686426 | TCTTGATCTTTGAATGAAGTTGG | 3254 |
| hs132685456 | CTACTAAATGGAACATTGAATGG | 3255 |
| hs132689748 | AATCATTGTGGCCAGCTCCCAGG | 3256 |
| hs132692021 | TTTTATTGCCAAAGTTTCCTAGG | 3257 |
| hs132685647 | CAAATGTTATGTTTGTTGCCAGG | 3258 |
| hs132693781 | ACAGTCCTTTTTATACATAGTGG | 3259 |
| hs132689101 | AACTAACTCATTGTTTAAAGAGG | 3260 |
| hs132683713 | TTTAATGTTTAAATTTTGAATGG | 3261 |
| hs132689183 | GGATTCCTTCTCTTACAAACTGG | 3262 |
| hs132689804 | AACTCAGGCATCATTCAGATTGG | 3263 |
| hs132694384 | TGTATCAGCTTGTTTCAGATAGG | 3264 |
| hs132687300 | TCAAACTTGCTGTAACCCTATGG | 3265 |
| hs132685534 | TGAATTCTAATGACTGCAATGGG | 3266 |
| hs132691591 | TCAAACTTGACCATATAAATCGG | 3267 |
| hs132683347 | TTATTTCTCATATTGACTAAAGG | 3268 |
| hs132683320 | ACATGGGCCTGATCTCACTATGG | 3269 |
| hs132689697 | TATAGACTCTGATTGAGAAATGG | 3270 |
| hs132689445 | TCTACAGTTCTTTTGCCCTCTGG | 3271 |
| hs132690816 | ATAAAACCTCAGTATGAAAACGG | 3272 |
| hs132684718 | TTATCAGGATATGTTTATAAGGG | 3273 |
| hs132688782 | CATTAACTGTTTCTATCAAAGGG | 3274 |
| hs132688377 | CACAACATTTAGCAACAGATTGG | 3275 |
| hs132684288 | GACGATGGAGTGCAGAATATGGG | 3276 |
| hs132691451 | GTAGGGGATTTGGGGTTGAATGG | 3277 |
| hs132692890 | AGAAAAAAGAAAAAAATAAGAGG | 3278 |
| hs132690423 | TCCCAGATCTGAGTCCTGTAGGG | 3279 |
| hs132687109 | GGGGCCACTAGTGTAAAAAAAGG | 3280 |
| hs132685746 | GGCCATCTCACAAAGCTCATTGG | 3281 |
| hs132691641 | CTCCGTTACACGTTTACAAAGGG | 3282 |
| hs132685378 | TCTTGTGGATTTACATTGCCAGG | 3283 |
| hs132693773 | TATTGACCTTTAATATAAAACGG | 3284 |
| hs132690897 | TTATACTTTGTTACTTCAAAGGG | 3285 |
| hs132685989 | AACTTTTTATTGTCACTAAGAGG | 3286 |
| hs132690997 | GGAAGCCCATAGCCTTCAATAGG | 3287 |
| hs132686041 | ACATCTTATGACAAATCAATAGG | 3288 |
| hs132690949 | GTGCTGCTGCCCCTCAGCATGGG | 3289 |
| hs132692600 | CCTGCTCTCGTCTTTATAAGAGG | 3290 |
| hs132683705 | GCTTATTTTAAATTTAATGATGG | 3291 |
| hs132691872 | TATGAAATATGTCAAACAACTGG | 3292 |
| hs132693277 | GTTCCCATATGAACACTGTTTGG | 3293 |
| hs132687923 | GCTAAAATGTAAGAGATGTTTGG | 3294 |
| hs132687642 | CACTTTCATTTATTCGGGGGAGG | 3295 |
| hs132683522 | AGAACTGTAGTTTCTCCATCAGG | 3296 |
| hs132685099 | CTACTTCTCAGAAGCTCTGTTGG | 3297 |
| hs132686860 | AGACAGGGCTTAGCTATATTCGG | 3298 |
| hs132691290 | CATTGAATGATATTTACAATTGG | 3299 |
| hs132688235 | CAGTTTATCTCCTAAATATGAGG | 3300 |
| hs132686034 | TTAAGATATTTAGTATATAGTGG | 3301 |
| hs132686738 | AATCAAGGCTAAGCTGCATTAGG | 3302 |
| hs132694153 | CTCCATATTCCTAAGCCAACAGG | 3303 |
| hs132688845 | CCCTGCAGAATTGGGATTGAGGG | 3304 |
| hs132687224 | GGATTTCAGTTGGAAGGCATTGG | 3305 |
| hs132686949 | CTTTTTGCTGATTTCCCTGTTGG | 3306 |
| hs132689165 | CAAAATGTATTGCTGTGAAAGGG | 3307 |
| hs132687066 | GGACATGGAACTTACTTGTTCGG | 3308 |
| hs132685940 | CAATATTTCTTTAGGTCAAAGGG | 3309 |
| hs132688489 | GCAACAGAATAGCAGATTAAGGG | 3310 |
| hs132692328 | CCTCCTCCAGGAAGATTTGAGGG | 3311 |
| hs132689899 | TGTTCTTTACACTTTTCAAATGG | 3312 |
| hs132686956 | TGTAAGAGAAAAGTACCAACAGG | 3313 |
| hs132690577 | TATTAAATAATCAAACAAGTTGG | 3314 |
| hs132694383 | GTATCAGCTTGTTTCAGATAGGG | 3315 |
| hs132685486 | TCACCGGCCATTTTCCTTTGAGG | 3316 |
| hs132684624 | GCAAACATTTTGATCAAAGTTGG | 3317 |
| hs132687615 | TTAGGTACTGTCATAGTGTTGGG | 3318 |
| hs132685127 | TTATCAGTTTGATTCATTAAGGG | 3319 |
| hs132683955 | TCCTTTGCATCTAATATGAATGG | 3320 |
| hs132692164 | TTTTAACTTAAATCATTGAATGG | 3321 |
| hs132688441 | GGAAGTGTGAATAAAAGTTAGGG | 3322 |
| hs132685854 | GCTCTTTACTCCAACAGAATAGG | 3323 |
| hs132685575 | TATTATGGATTTACAAAATGAGG | 3324 |
| hs132687229 | TTAATTTCCTCCAAAGCCTTGGG | 3325 |
| hs132691388 | ATTATATTAAAAAACTAGATTGG | 3326 |
| hs132685601 | AAAATATCCCTGATTATCATAGG | 3327 |
| hs132694160 | GTAACAGACACAAGGCTTAAGGG | 3328 |
| hs132687230 | TTTAATTTCCTCCAAAGCCTTGG | 3329 |
| hs132683962 | TTTGCCCCGGAGCCCGGCATTGG | 3330 |
| hs132686266 | TAAGTATTAGTTCTGTTGGTTGG | 3331 |
| hs132683439 | TTAGAAAGCTAATTTTATATGGG | 3332 |
| hs132685240 | CTCTCTACTGAAGTGATTGAAGG | 3333 |
| hs132689904 | CACATTACTTGGAAATTAAGTGG | 3334 |
| hs132689268 | GTTAAAAGGGAGCCAGAATAGGG | 3335 |
| hs132691869 | CATCGGTCTCTAAATAAACTTGG | 3336 |
| hs132686995 | CAAGCACTGTTGGTTTTCATAGG | 3337 |
| hs132689266 | CTGTGTTTTGATAGTTAAAAGGG | 3338 |
| hs132683403 | ATATTTAGCAAAAGTCATAAAGG | 3339 |
| hs132692310 | TGCCAGGGAGATGAAAATTAGGG | 3340 |
| hs132685132 | TTATAAGCCGGTCTCACAATTGG | 3341 |
| hs132693914 | TGTATCTTTTTTTTCCAAAACGG | 3342 |
| hs132685073 | GTAAAATCTGATTTTCTAAGTGG | 3343 |
| hs132687427 | CTTTTTATAGTGGTGGGGGGAGG | 3344 |
| hs132691637 | ACTCAGTTATAGGAAGTAACAGG | 3345 |
| hs132683291 | AAAACACTAACACATCATAATGG | 3346 |
| hs132686741 | CTCATTACTCTTTAACCAAAAGG | 3347 |
| hs132685690 | TGTTCATTAATGACTGGCTCTGG | 3348 |
| hs132690136 | TACATTCTCATTTCATTACATGG | 3349 |
| hs132683932 | TGGTGTCTAGCAAAGTTAAAGGG | 3350 |
| hs132685315 | GTATAGAACTTTGTATTGTATGG | 3351 |
| hs132693103 | GTATTATATGTAAGTCTCTTCGG | 3352 |
| hs132691300 | AATTTACTCTCAAATTAAGTTGG | 3353 |
| hs132692355 | GCTCTCCCGCACAATGGCATTGG | 3354 |
| hs132685266 | ATTTTTTTAGTGCAGTGTTTGGG | 3355 |
| hs132689082 | GTAAAATAAATATTTAGAATGGG | 3356 |
| hs132693250 | CTGTTGTTCCTTAGTATTGAGGG | 3357 |
| hs132690549 | GAACTGGAGTTCAATTTGTTCGG | 3358 |
| hs132689996 | ATACAGTAGTCAGTTGAAATGGG | 3359 |
| hs132687908 | CCTGTTCATGTATTTTTGAATGG | 3360 |
| hs132688786 | TGTTGATCAGTCTCACCTTATGG | 3361 |
| hs132694356 | CTATGTAATACAGAATCTTTCGG | 3362 |
| hs132693903 | GAAGTAATCAACCTTGGAATAGG | 3363 |
| hs132684923 | ATATAAATTATACATTTAAGAGG | 3364 |
| hs132684014 | CTTTTCATGACTTTTCCCATTGG | 3365 |
| hs132691024 | TCAAGTTGTTTCCTGGGTGTGGG | 3366 |
| hs132688908 | TATTTTTTAAACAGATCAACAGG | 3367 |
| hs132687652 | AATGCATTTCAATAAATATGAGG | 3368 |
| hs132686772 | GGCCACACTTGGAATTGAAATGG | 3369 |
| hs132685304 | TTACAGCTTTGTGAAACAATTGG | 3370 |
| hs132686607 | GTATTTCAGGATTTCACATCAGG | 3371 |
| hs132688824 | TTAGTCTTATCCATCTTGCTTGG | 3372 |
| hs132684935 | GTTTTAATCTGTTATACTGCAGG | 3373 |
| hs132685051 | ACCGTCAGAGTAAGTCTTAAGGG | 3374 |
| hs132683391 | TGAGCCTTCTTTAATGGTGCTGG | 3375 |
| hs132683876 | ATGTTTCTCAAGGCAGGTTAGGG | 3376 |
| hs132687020 | CCAAGAACTGCAGCTCTACTTGG | 3377 |
| hs132688264 | TGATAAATGAGGTCAGATATTGG | 3378 |
| hs132689818 | ACTTATTGTTGGCTTTAAAAGGG | 3379 |
| hs132694306 | CGAAGGAGACAATTCACAAAAGG | 3380 |
| hs132687701 | AACTATCTATTAATATCATTCGG | 3381 |
| hs132683690 | ATAATAATACTGATCTCTATTGG | 3382 |
| hs132686671 | TAGTGAACCTGCAAATCAATAGG | 3383 |
| hs132686461 | CTAACTCTTAACCATTTTGAGGG | 3384 |
| hs132684976 | GTGCATTTGCCCCTAGTTAAGGG | 3385 |
| hs132685886 | GCAAGAAATTTCATGTAGTTGGG | 3386 |
| hs132687620 | GCTTTTGTCAAGCTAAAATCTGG | 3387 |
| hs132690535 | TCAGGGCATTCAGATCTATCAGG | 3388 |
| hs132693957 | GGGAAATTACTAATGAATGTTGG | 3389 |
| hs132693148 | ACTGGCACTTTTTAGCTGTAAGG | 3390 |
| hs132690665 | CCTTGATCATATTTTCTACAAGG | 3391 |
| hs132692987 | TTTCTAGCCATTCTGACAATTGG | 3392 |
| hs132690866 | CCAAAACAAATCAGTTTAAGAGG | 3393 |
| hs132683476 | CAGAAAATGTATCTTTAAAAGGG | 3394 |
| hs132689106 | CCTGGGGTTATCAAGATACTAGG | 3395 |
| hs132692696 | TTTTGTTCTGAAATTTTCCTTGG | 3396 |
| hs132687060 | GTAATTGTTTCACATTTAAGAGG | 3397 |
| hs132690106 | TTGCAACCGTAATCTGATAAAGG | 3398 |
| hs132690872 | TTTCTCATGGTACTTAACTAAGG | 3399 |
| hs132691472 | GCTTAGGTATTTCCTCATAAGGG | 3400 |
| hs132690656 | TTCAGTTGTATTAGTGGCAGTGG | 3401 |
| hs132689313 | GGGGGCGTGGGGCCAAACAGAGG | 3402 |
| hs132690642 | GGAAATTATGTGACACCAGCTGG | 3403 |
| hs132691576 | ACTTAGATTTGTTAAATATGAGG | 3404 |
| hs132685236 | AGGTCCTTATGGGTGTTTGATGG | 3405 |
| hs132687884 | CATTTGCAAAACAATTTAAGAGG | 3406 |
| hs132683528 | GGTAGATGTATATAAGGTTTGGG | 3407 |
| hs132692832 | GCCACTGCAGTCCTGAAGGTTGG | 3408 |
| hs132687228 | CACATTTCCCAAGGCTTTGGAGG | 3409 |
| hs132689187 | TTTTTTCTAAAACAGTAGATTGG | 3410 |
| hs132689029 | ACTCCAATTGTTATAAAAGAAGG | 3411 |
| hs132693916 | TTTAATGATATTGGAAAAAGAGG | 3412 |
| hs132686216 | CTTCCAACAGAGATTTTAGTTGG | 3413 |
| hs132692838 | AGTTTAATATATTTGATACTAGG | 3414 |
| hs132692085 | AAAAGAAAATTATTGTATAAGGG | 3415 |
| hs132685309 | CACTTGTAATCCATCCTTAAGGG | 3416 |
| hs132687582 | TGATTTCTTCAAAGGCCTGCTGG | 3417 |
| hs132690165 | GAACCCCATTCCTCACTAATGGG | 3418 |
| hs132687741 | TTAATCTATGATGAAGGAAATGG | 3419 |
| hs132686286 | AAGCTGATTGATTCATTAAGAGG | 3420 |
| hs132683782 | GTTTGTTTATTTGTTTCTGCAGG | 3421 |
| hs132690303 | AGAAAATTATCTTTAAAGAAAGG | 3422 |
| hs132688399 | TCTGAGCTTTAAGCTATCATTGG | 3423 |
| hs132684524 | TTATATGCATATATACAGTTAGG | 3424 |
| hs132683364 | TAGACTTTATGGAGGAAGATTGG | 3425 |
| hs132690624 | AAGATTTCTGAATGTAAGAAAGG | 3426 |
| hs132690915 | CCATATGATCTAAAATCTATTGG | 3427 |
| hs132690337 | ATACAGTGATCTCTTTTAAAGGG | 3428 |
| hs132691775 | ATATGCTCATGAGCTTCCTTTGG | 3429 |
| hs132685675 | GGAAAGATTTGAAAATTCATTGG | 3430 |
| hs132691141 | ACCAGTGGTTGACAGAAGTATGG | 3431 |
| hs132693682 | AGATGGAAGCCAGCATCAATAGG | 3432 |
| hs132692825 | GTATCTTCACCCCAACCTTCAGG | 3433 |
| hs132692366 | AAGCTTTTGTGCAAATTAGTTGG | 3434 |
| hs132692051 | GCCTCCCCTTATTTTTATGTAGG | 3435 |
| hs132686474 | GCGTAATGAAAAACGATAGAAGG | 3436 |
| hs132690876 | TATTTTGCAACAGTATAATAGGG | 3437 |
| hs132688153 | GGACCAACACAGAAATATATAGG | 3438 |
| hs132691887 | AAGCAAATCATCAGACTTAAGGG | 3439 |
| hs132690070 | GAAGAGCTAGAGGATTCCTTTGG | 3440 |
| hs132689446 | ACTGTAGACGGGTAAATAAAAGG | 3441 |
| hs132685252 | TTAAGGCTTTTATAATCATTCGG | 3442 |
| hs132689280 | TGAGGAAAGCTGGGTTTTAGTGG | 3443 |
| hs132689864 | ACCACAAGTGAAAAGCAGTATGG | 3444 |
| hs132687609 | GTTTTTGTGGATTCACTGCTTGG | 3445 |
| hs132693558 | CCCTTGTCTGATTTCAGTAAAGG | 3446 |
| hs132686362 | CATGGTTGCCTATTGACTTAGGG | 3447 |
| hs132686961 | GTGCCTGCTTTATCTGTGCTTGG | 3448 |
| hs132691162 | GAAACTCTGTTCCTGTAAACAGG | 3449 |
| hs132691792 | CTTTGTATTTTGTCGATGATAGG | 3450 |
| hs132686398 | AATCTCAATTGTTTGCCAAATGG | 3451 |
| hs132687408 | CCAAGACTATTACAATAAAACGG | 3452 |
| hs132692742 | AATTTGCATGAATATACAAATGG | 3453 |
| hs132692642 | AGGAGGCTCTTTCACTTTTGCGG | 3454 |
| hs132684535 | CTAAACAATTTTCAGTGTAAAGG | 3455 |
| hs132684187 | CCTCAAATAATTAGAGCAACAGG | 3456 |
| hs132688357 | TGATATGTCAGGCCAGTAACAGG | 3457 |
| hs132693843 | ATAGTACTGCTCGTGTACTATGG | 3458 |
| hs132694190 | CACTATAAAAGAACAGTTAAGGG | 3459 |
| hs132684186 | CCTGTTGCTCTAATTATTTGAGG | 3460 |
| hs132694361 | CCATGACAGCATGTGAAGATAGG | 3461 |
| hs132683724 | CACTCCCTGTTCACCAAATACGG | 3462 |
| hs132694243 | GCTTTTTCCTTCTTAAAAGATGG | 3463 |
| hs132692086 | TGGCTCTCAAAAGATAGTTTGGG | 3464 |
| hs132687905 | CTCTGGCATGGAGTTCAAATAGG | 3465 |
| hs132688682 | AGAAAAGGAGATTCAAGATATGG | 3466 |
| hs132693350 | GTATCTGCTAAGACAAAATCAGG | 3467 |
| hs132688020 | GATTTCAAGGGGAAAAATTAGGG | 3468 |
| hs132690271 | TCAAGGCAGTTGGTTCGTATTGG | 3469 |
| hs132684666 | ATAAGGAAAGAGGAAATATAAGG | 3470 |
| hs132689671 | TCACTCCATTTTTCTCCAATGGG | 3471 |
| hs132684647 | CAAAGAGGCTAGAGATTGATTGG | 3472 |
| hs132685612 | TATGTTCCCATTTTAAAAGTAGG | 3473 |
| hs132687574 | ATATATCAGTAACTTTAAATGGG | 3474 |
| hs132690206 | ATTTGGCTCCAGCGTTTTAAGGG | 3475 |
| hs132692191 | AAGCAGATAGAATCGTGAAATGG | 3476 |
| hs132691147 | AGTTTGCTCAGAACTATATAAGG | 3477 |
| hs132693791 | TCACAAAGCTATGCATTAGAAGG | 3478 |
| hs132691582 | ACATTTATTTAGAAATGTAATGG | 3479 |
| hs132692584 | CATATTATCGTGATGTAGATAGG | 3480 |
| hs132688883 | CATTGGTCTCTGTATTTACAAGG | 3481 |
| hs132686104 | TACTTCCTCATTTTAGAAAAGGG | 3482 |
| hs132693566 | CTATTACTGTATTGATTAAGTGG | 3483 |
| hs132686968 | ACCTCACTATTCACGGCAACCGG | 3484 |
| hs132689430 | GAAAAAATCTATGGTATAAGAGG | 3485 |
| hs132693094 | ATAATTTTGTAAAATCGAATTGG | 3486 |
| hs132687119 | GTAAGTTTGTCTGCAGGGCTTGG | 3487 |
| hs132690483 | AGAGTATTCTTTATTTTAGCAGG | 3488 |
| hs132685579 | ACTTACACTATCTTACCAATAGG | 3489 |
| hs132688039 | ATCCTCTTAGAAATGACAATTGG | 3490 |
| hs132684015 | TTTTCATGACTTTTCCCATTGGG | 3491 |
| hs132691614 | CTAGCATGCTATACATCATATGG | 3492 |
| hs132690957 | GCCTGTTCCATGTTTGGTGTGGG | 3493 |
| hs132683645 | GTATATTTATCTATTTTTATGGG | 3494 |
| hs132690039 | GGAAGGGAAATTGGAACAACAGG | 3495 |
| hs132692677 | TCTCGTCTTCCTGTTGTCTTCGG | 3496 |
| hs132693013 | GTTTTATCATTGCTTTTTTGTGG | 3497 |
| hs132686557 | ATAAATGAGCTGGCCTGTATAGG | 3498 |
| hs132687436 | TTGGTCCTCTTGGATGTAAAAGG | 3499 |
| hs132686156 | GTTAAGTGAACGCTTTTCATTGG | 3500 |
| hs132684156 | TCTCAACAGCCTAGGATGATTGG | 3501 |
| hs132684682 | TGAGCAGTTAAATATTTAAAGGG | 3502 |
| hs132686357 | TCTCTCTTGTGATGTGATAAGGG | 3503 |
| hs132687599 | CTATAAATATAGAGTAGAAATGG | 3504 |
| hs132689787 | TTACAAAGCTGAGATATCTTAGG | 3505 |
| hs132694020 | ATCCTGTCTGGATGACCTAATGG | 3506 |
| hs132686497 | TCCACTTCATACTTTCAGTTAGG | 3507 |
| hs132693716 | GCACATACGGCATCAAATAATGG | 3508 |
| hs132690493 | CCTGTTGCATAAAAGTAATAAGG | 3509 |
| hs132689654 | TAAACCAGAATTTAAATTGAGGG | 3510 |
| hs132685310 | ACACTTGTAATCCATCCTTAAGG | 3511 |
| hs132690229 | CTTGGAACTATATTTAGTATAGG | 3512 |
| hs132690929 | TCCTAAGTCAGAAGTCTATTGGG | 3513 |
| hs132691504 | GCCTCTCGATATTTTACAACAGG | 3514 |
| hs132683587 | TCAATTGCTTTTTTATTGTAGGG | 3515 |
| hs132689447 | TTTTGCCCTCTGGTACTTTGTGG | 3516 |
| hs132692936 | AATGATATGGTCTTAATAGAAGG | 3517 |
| hs132685460 | TCTTAACCATCACATGCCTTTGG | 3518 |
| hs132687777 | TCTTTTGTCTGTTATTTCCCTGG | 3519 |
| hs132687458 | GCCTAAAGCACTGAGAATTAGGG | 3520 |
| hs132685375 | TCTTGTTTCATAAGAAAAACAGG | 3521 |
| hs132688917 | ATACGTTATTTAGCTCCCTTAGG | 3522 |
| hs132693543 | CTTTTTAACTAACAGACTAATGG | 3523 |
| hs132684144 | AAAGTTCTCTTTTTTTTCCATGG | 3524 |
| hs132690225 | ATAATATGCTGTAATATATGAGG | 3525 |
| hs132687558 | GCTATCCTGCAGATGCTAATGGG | 3526 |
| hs132687532 | ATTTTAGGTTGTTTTTATAAGGG | 3527 |
| hs132690257 | CTACAAACTATTTATTTGCTTGG | 3528 |
| hs132693158 | AATAGATTCCAAGTATGTATTGG | 3529 |
| hs132686893 | ATATCTCAGAATGATTTTAGAGG | 3530 |
| hs132690940 | AGCTTAGCGAACAGTGGCCTGGG | 3531 |
| hs132684979 | TTAAGGGTCAACGAAATCTAAGG | 3532 |
| hs132685930 | GTTTGATTTAGCACTCTTAAGGG | 3533 |
| hs132692924 | TCATTACAGCAGCTACAAAAAGG | 3534 |
| hs132686887 | AATATACTGACAGTATATATTGG | 3535 |
| hs132688674 | GCTGTCTAAATGCCTCAAAATGG | 3536 |
| hs132692055 | CAATACCTACATAAAAATAAGGG | 3537 |
| hs132685671 | GTGTAATTGCTAAATATTGTAGG | 3538 |
| hs132684521 | GTAGGGATTAGTTTTAAATAGGG | 3539 |
| hs132687941 | TATTTTTAAGTATTATTAGTAGG | 3540 |
| hs132692020 | ATTGAAATCCTAGGAAACTTTGG | 3541 |
| hs132690585 | TTTTCCACAGTGATCCATATTGG | 3542 |
| hs132684742 | TCAAACGTGCAAAATATGTTTGG | 3543 |
| hs132692919 | GTGTGTGTACATTCACAATTTGG | 3544 |
| hs132692402 | ACTATATATCCCATTTTAAGAGG | 3545 |
| hs132692891 | AATTCCAGTTTTATCATGATAGG | 3546 |
| hs132690905 | TTTCTCTTCTTAAAAGCTAAAGG | 3547 |
| hs132689691 | AATTAAGTGTCATCATCTAATGG | 3548 |
| hs132687723 | GATTTTCCTTTCATGGACTAAGG | 3549 |
| hs132686978 | GATTAGAAGGGAACGTAATCAGG | 3550 |
| hs132687117 | GCTTGGTCTGATTGATTTGAAGG | 3551 |
| hs132688129 | GACCCACAATTCAAACGTTTGGG | 3552 |
| hs132693552 | TTAACAGTATTAAGATCATAAGG | 3553 |
| hs132684759 | TGTTGCCTGTAGTAACAAATAGG | 3554 |
| hs132684723 | ATTCTAGGTATGTTCCAAAAAGG | 3555 |
| hs132683912 | AGGAATTCACAAACACAGATAGG | 3556 |
| hs132683879 | CTTAGAATAAACGTATCCTTTGG | 3557 |
| hs132691992 | AAAGTCCCCCTAAGTTTTAGAGG | 3558 |
| hs132688490 | GGGCAGCTTTCTGAACTGATTGG | 3559 |
| hs132691858 | AGAAATCTGTGAATAAATAAAGG | 3560 |
| hs132694036 | ATCTCAGGTTCCTCTCCCTTTGG | 3561 |
| hs132686135 | GTTCACTGAGCATAATGTATTGG | 3562 |
| hs132690770 | CAGAATGCCTTAAAAAGAAATGG | 3563 |
| hs132690010 | GGAAATTAGGGGTAAATAGATGG | 3564 |
| hs132683493 | GTGCCATTAATATTACAAAATGG | 3565 |
| hs132693968 | TTAATTTCCTAGGAGGAGTTGGG | 3566 |
| hs132687036 | TCAGATGCGGAAATTTCATTTGG | 3567 |
| hs132688342 | GTGCTCCAAAAATTTTTAGTAGG | 3568 |
| hs132687977 | TTCTCATTAACAAATGTAGATGG | 3569 |
| hs132686338 | GGGGAGGTGAAAAAGATTTACGG | 3570 |
| hs132692317 | TCATAAGCCCCAAATTTTGATGG | 3571 |
| hs132686565 | GCTGAAAGCCTTGGAACTTTCGG | 3572 |
| hs132691650 | TGCTCTTTTGGTGTGATCAAGGG | 3573 |
| hs132693117 | GAGAGAATCTGTTTACATATTGG | 3574 |
| hs132688444 | ACATCCATCAAAAAACTCTTTGG | 3575 |
| hs132691987 | TTACTGACCTCTAAAACTTAGGG | 3576 |
| hs132692177 | TCAAAGAAGGAAGAGTTCATTGG | 3577 |
| hs132693517 | TTTTTCCCATTATTGCTAGAAGG | 3578 |
| hs132685953 | GTATAGTTCTATGTGCTATAAGG | 3579 |
| hs132683971 | TTGACTCCCAGCTCAACTTTGGG | 3580 |
| hs132690461 | TTAAAAGTCAATTTTGAATCCGG | 3581 |
| hs132692665 | AACTTCATCCTAAGGTCAATAGG | 3582 |
| hs132687895 | CTATTTTGAAGCAAAGGTTTAGG | 3583 |
| hs132693395 | TTATGCGTAGGGTTCTGTTTAGG | 3584 |
| hs132691221 | GTACGTATTTAAAAACGATTAGG | 3585 |
| hs132685530 | TATGGTAAAATAAAGTATAAGGG | 3586 |
| hs132687682 | TCATTTTATTCCCTTATTTAGGG | 3587 |
| hs132686547 | TGAATTGAATTTTTGAAGATGGG | 3588 |
| hs132690873 | GCTAGTTTATGTGCTATGTTAGG | 3589 |
| hs132685075 | TGCTCTGTGAAGAAAGAAATTGG | 3590 |
| hs132686269 | ATTGACTATGAATTTATAGATGG | 3591 |
| hs132683659 | TATTTACTCAACTTACACTATGG | 3592 |
| hs132688624 | CTGTCAACTCTCTAGAAGATAGG | 3593 |
| hs132691209 | TTTTTACAGGAAAATACATTAGG | 3594 |
| hs132685604 | CTGTCAACAGATAATTTAGAAGG | 3595 |
| hs132691702 | GCTATGGCACCTTCAGTGATAGG | 3596 |
| hs132689847 | GCAATCCATTTCTATTATATGGG | 3597 |
| hs132685670 | AACCAGCCCCAGAAGTAAACTGG | 3598 |
| hs132692846 | AGCTAGTCTTAAAATCAAAAAGG | 3599 |
| hs132686560 | TGCTGGCTATACACTTTTAAGGG | 3600 |
| hs132686363 | TCATGGTTGCCTATTGACTTAGG | 3601 |
| hs132691784 | TCAAATAGATACCTTTTAAAGGG | 3602 |
| hs132693992 | TTAATATCATTGTATGCAACTGG | 3603 |
| hs132689764 | GCTCCTGCTTGAGCTATTGTTGG | 3604 |
| hs132687274 | GGAAGTCAATTTAAAATAAGAGG | 3605 |
| hs132693956 | TACTTTCCTTAATCTATAAAGGG | 3606 |
| hs132684231 | AGTAGTCATTCTAAAAAGATTGG | 3607 |
| hs132684710 | TGGCTGTTAATATCTCAAAAAGG | 3608 |
| hs132690340 | ACTACGGCAAATTTACAAACTGG | 3609 |
| hs132691527 | TTATAAACTGGCAAGAATATCGG | 3610 |
| hs132688152 | TGTTGGTCCTTCATAATACTTGG | 3611 |
| hs132683489 | GATATTTTGGAGCATTATAAAGG | 3612 |
| hs132684933 | ACATTCAAAGTGATGAGCTTTGG | 3613 |
| hs132687464 | ATTTGCCAGCATTAATAATAGGG | 3614 |
| hs132687150 | AGGGTAGCTGTATAATTAAAAGG | 3615 |
| hs132691579 | ATAGAGAATTATTATATCATTGG | 3616 |
| hs132685247 | GTTCAGAACCACGCACTTGTAGG | 3617 |
| hs132690294 | AGAGTTAAAAGAATTTATAATGG | 3618 |
| hs132686745 | TGATTTCTACCACAGCCTTTTGG | 3619 |
| hs132693361 | GCTGTAATGTTGACAATATCTGG | 3620 |
| hs132685960 | CTTTAAGGAAAGTTTTTAAAGGG | 3621 |
| hs132686695 | TATCTTGCTTTGCTATAAACAGG | 3622 |
| hs132689432 | GTAGGTTTAGAAAAAATCTATGG | 3623 |
| hs132693884 | TTCACTGTATGTGAACAAATAGG | 3624 |
| hs132689038 | TTAATTTATGAGCCTTTGTCTGG | 3625 |
| hs132689956 | TATAATATGCCAATGTCTATAGG | 3626 |
| hs132691889 | AAGTCTGATGATTTGCTTAAGGG | 3627 |
| hs132686323 | CGGCTGTTATTCAGTTATAATGG | 3628 |
| hs132686574 | TACCATACGCTAATCAACTATGG | 3629 |
| hs132690419 | TTAAAGCTGCTGAAGTTTGTTGG | 3630 |
| hs132690187 | TGTTTAGGCTGCCATTTTAAGGG | 3631 |
| hs132688062 | TTTTCCCTCCCAAATCATAAAGG | 3632 |
| hs132684226 | AGGTTTCACTGAACCATTAGAGG | 3633 |
| hs132694127 | ATATTCTTACCCTTGGAATCAGG | 3634 |
| hs132693885 | TGCTTCACTTACTCCTTAAGAGG | 3635 |
| hs132685751 | GTATATATACGTGGCTTATGAGG | 3636 |
| hs132693549 | GGTAACTGAGGTGTTCAATATGG | 3637 |
| hs132689149 | TTTTCCTGACCAGAAATAACAGG | 3638 |
| hs132691521 | ATGGCTAGTTTCTCATTAAAGGG | 3639 |
| hs132687719 | AAGCAAATTTTAGAAATAAGAGG | 3640 |
| hs132690401 | TATGTAACTGTTACTAAATACGG | 3641 |
| hs132684470 | GATTTTCAGCACCTGCTATTGGG | 3642 |
| hs132683600 | CCATAACTCCAATTTGCTAATGG | 3643 |
| hs132686384 | AGAACCGTGCCCAATACTTATGG | 3644 |
| hs132692068 | GTAGCTATAAGTGATTTAACAGG | 3645 |
| hs132690586 | ATATGGATCACTGTGGAAAAAGG | 3646 |
| hs132688828 | CTTTATCGTTTAGTTATTAAGGG | 3647 |
| hs132683925 | GGAGCATAGCTATTCCTAAATGG | 3648 |
| hs132691457 | GATTTGGGGTTGAATGGGGGGGG | 3649 |
| hs132689824 | CCAGGATTACTTGCCTTTGTAGG | 3650 |
| hs132684560 | ACAGTTCCCAAGGATTTGTTTGG | 3651 |
| hs132689295 | ATTCCAGAACAAATTCAATATGG | 3652 |
| hs132685847 | TTTCTGTTCCTTCCCACAATAGG | 3653 |
| hs132692345 | GACATGGGCAACAAATTAGATGG | 3654 |
| hs132689288 | TGTGCCTTATATTTCAAAATCGG | 3655 |
| hs132690542 | CACAATGCAGAAATCATTATAGG | 3656 |
| hs132684748 | CCATCACTTTCACAAAACTATGG | 3657 |
| hs132689353 | TATATGGGAAAGGTTACATTAGG | 3658 |
| hs132689679 | CCTGCAATTGGAAAGATACTTGG | 3659 |
| hs132685627 | GAGTAATTAAAAGTGCTTTAGGG | 3660 |
| hs132686598 | GACACAGTCTGAATAGTAATTGG | 3661 |
| hs132685119 | TTTATATCCACTATTGTGATTGG | 3662 |
| hs132693270 | GCCTTTGGAGAGGATGCATTCGG | 3663 |
| hs132693628 | CACAGTTTACTTTATGCAAATGG | 3664 |
| hs132691780 | CTATGTTACTGAAAAGTAACTGG | 3665 |
| hs132688950 | TTAGCTCTTCCAACAATAACAGG | 3666 |
| hs132690470 | CCAAAGTTATTTTTTCCCCTTGG | 3667 |
| hs132688410 | GCAATAGTCCGTAAACTTCCGGG | 3668 |
| hs132690685 | ATATGCTCCCTCATTCTTCTGGG | 3669 |
| hs132693294 | TTCTTCTCACTGAATATTAAGGG | 3670 |
| hs132684597 | AGTATTTAGAGCAAAAAAAAAGG | 3671 |
| hs132690563 | TCCTTGTGTTATTAAATAGATGG | 3672 |
| hs132688055 | ATTACTTTCATTAAAAGAATAGG | 3673 |
| hs132693762 | ATGGCACTATTGCCTCAAAATGG | 3674 |
| hs132685216 | TATGCTTTGTTTTTTCTCAATGG | 3675 |
| hs132689338 | GCAGTAGCTCAGAATGAGATAGG | 3676 |
| hs132689322 | GCACAGTAATTATTCATTATGGG | 3677 |
| hs132686984 | TCTGAGTCTGAAGCAGAAACTGG | 3678 |
| hs132693358 | GGCTTATGATGTTAATTTAGTGG | 3679 |
| hs132693346 | GTTCTTTCAGAACATAATAATGG | 3680 |
| hs132688143 | GAGAAGCTAAAGAGTAAATCAGG | 3681 |
| hs132683350 | GATAAATCTTAGCTACAATATGG | 3682 |
| hs132688664 | TTAGCACCTATGTCAGTAATTGG | 3683 |
| hs132688093 | AGTATTCTCAGAGATGAAAAAGG | 3684 |
| hs132688528 | CCTCTTTTCAGTTCAAAGAATGG | 3685 |
| hs132694023 | ACAGAGTCTATTTCTCCATTAGG | 3686 |
| hs132688429 | TTAGAGTCAGTGAGTTTATTAGG | 3687 |
| hs132688654 | ATATGGATTGTCTAAGTCATTGG | 3688 |
| hs132689959 | AAACAACCCAAAATTTTAATGGG | 3689 |
| hs132689682 | TTTCTAATAGAAGTATAAAAAGG | 3690 |
| hs132690138 | GAGAATGTAGATGGCTTAATGGG | 3691 |
| hs132683832 | ATACCACGCTTTGAAAATACTGG | 3692 |
| hs132687734 | ATAAAAGGAAACTTTTTAGATGG | 3693 |
| hs132691298 | TAGTTATGTAAATTATTTTAGGG | 3694 |
| hs132693656 | AGCCACACTGAAAGAATATATGG | 3695 |
| hs132690832 | ATATTTAGCTATAGTCGTTTAGG | 3696 |
| hs132692243 | GCATTTACCATGCAAGAAAAAGG | 3697 |
| hs132687510 | CCTTTGCCGATCAGCTTTAAGGG | 3698 |
| hs132690367 | TCCACAACTACTAAAAAAAAAGG | 3699 |
| hs132692334 | GGATCCTTGTTATAAACGTTTGG | 3700 |
| hs132691914 | TTGTTTTTAGAATTATTATCTGG | 3701 |
| hs132692633 | ATAGAAGCTGAACTCTAAACTGG | 3702 |
| hs132686317 | GGATGAACTTCATTATAATCTGG | 3703 |
| hs132683931 | AGATGACATGTCTAACAAAATGG | 3704 |
| hs132688867 | ATGCAGGGTGGTCAGGTAAAAGG | 3705 |
| hs132689225 | CTTGTGGTGCTGCTGAGATTTGG | 3706 |
| hs132691857 | ACTTTGCTGTTGTCCCCATCTGG | 3707 |
| hs132688914 | TTAGGGATATGATTACTCTTGGG | 3708 |
| hs132691271 | GCTCCCAAGCTACCTTTAACTGG | 3709 |
| hs132688537 | TTATTTGATAGACTAGTAAAAGG | 3710 |
| hs132683304 | TTTACTCCGAAAAACTATTTCGG | 3711 |
| hs132687931 | TTAATTATCACACACTATAAAGG | 3712 |
| hs132694409 | TCAGGAGTCTAGGTTTCTTCAGG | 3713 |
| hs132693062 | GCCAGGCATTGTCTGATTTAGGG | 3714 |
| hs132686069 | ATGTATTTCTTTGTTAAAGATGG | 3715 |
| hs132686077 | TGATATTAACATGAAAAAATTGG | 3716 |
| hs132685194 | CCCACATTGCTGGTACCGTTGGG | 3717 |
| hs132692800 | CTCTAGGCTAAACATATAAAAGG | 3718 |
| hs132689536 | CATAGGAGCCTTTGGCTAAAGGG | 3719 |
| hs132691344 | TCAATTCCTTGCATTTTTGCAGG | 3720 |
| hs132686914 | AGTGATTTGCATTATCCTATAGG | 3721 |
| hs132686759 | AAGAGAAGTGGAAGGTTTAGAGG | 3722 |
| hs132689861 | GATAGATTAAGGAAAGAAAAAGG | 3723 |
| hs132691186 | CTACCCGAAATATCATTCTTAGG | 3724 |
| hs132691516 | AATTTATTCATGTATAAAACTGG | 3725 |
| hs132693834 | CGGTGTGAAATAGACAAAAATGG | 3726 |
| hs132690610 | GCCATGGGCTTCATTGTCTATGG | 3727 |
| hs132684230 | TTGACTGCCCAAACCTCTAATGG | 3728 |
| hs132685294 | GCACAGAATCATTTTTATATAGG | 3729 |
| hs132692729 | GGTAGATGTATTTTTAAGCTAGG | 3730 |
| hs132685868 | GCCAACATGCAATAAAAGTTTGG | 3731 |
| hs132686992 | ACACAGGTGCGCAACTTTATAGG | 3732 |
| hs132693215 | CTTGGAATGCAATCTTTCTTGGG | 3733 |
| hs132690003 | TTTCTACTAAGGCAACATTTTGG | 3734 |
| hs132685633 | AGAGCGATAGAGGGAGATAAAGG | 3735 |
| hs132687406 | CCTATTTCTATAAACTTTAAGGG | 3736 |
| hs132692922 | TATGTTATTGAAATCATTTAGGG | 3737 |
| hs132691686 | TTCATCACCGTGATTACTAATGG | 3738 |
| hs132688571 | ACAATGTAGTGAAAATAAATTGG | 3739 |
| hs132684374 | CACACACCTTTTCAAATAAGAGG | 3740 |
| hs132691693 | TGTCCTTCAGATTTAGTTAGTGG | 3741 |
| hs132685452 | TTCTATTTAGTGCCATTTAGAGG | 3742 |
| hs132689822 | ACTGTCCTCAAGAGGAAATTAGG | 3743 |
| hs132691470 | CATTAAGCTAAGCAAATAAATGG | 3744 |
| hs132690107 | TCTCTTCCTTTATCAGATTACGG | 3745 |
| hs132693760 | CATTTGTGCTGTCAAATTAAGGG | 3746 |
| hs132691317 | CTAGATAAAATTCAATTGATAGG | 3747 |
| hs132685147 | ATTTTCTTTGGTTATATAAAGGG | 3748 |
| hs132687352 | AGTCCCAGTTATAAATTAATGGG | 3749 |
| hs132685495 | GTATACCAATTGTTCAGTTTTGG | 3750 |
| hs132685806 | TTTTATTTACTGCAGCTAAAAGG | 3751 |
| hs132683480 | ACATTTTCTGTGTCACATTAGGG | 3752 |
| hs132692490 | GCTCTCTTCAAATTTGGTATAGG | 3753 |
| hs132687270 | GCACTGTAAGGTACTTTATTGGG | 3754 |
| hs132684962 | TGTCCCCAGGAATTCTATAATGG | 3755 |
| hs132685027 | TTAAAACTCAAATTATTTGTAGG | 3756 |
| hs132690649 | CAGGGCAGAGAATTGCAAAATGG | 3757 |
| hs132685516 | CATCTTGTATGTAATTTTAAGGG | 3758 |
| hs132691524 | GGAAAATATTTCATTTTTAACGG | 3759 |
| hs132688451 | GCCACCAAAGAGTTTTTTGATGG | 3760 |
| hs132684236 | GTTTCTTTCACTGTGGTAACTGG | 3761 |
| hs132686490 | GCAGATTTAGCCAAATTAACAGG | 3762 |
| hs132687081 | TTTACAGAAACTCATATATAGGG | 3763 |
| hs132686120 | TTACTGTCTTAGAAAAGTTTAGG | 3764 |
| hs132687697 | AATACCAGATGCCACATAAAAGG | 3765 |
| hs132690636 | TCAGAGGGCAAGGGAAAAAAAGG | 3766 |
| hs132688887 | AGATGAACAATCCAGATATAGGG | 3767 |
| hs132687600 | TATAGAGTAGAAATGGAAATTGG | 3768 |
| hs132685044 | CTCAGTACTTAACATTTTAAGGG | 3769 |
| hs132692736 | AGGCATTACATGGGGATAAATGG | 3770 |
| hs132694224 | TCTCTGCTCACAACATTAACTGG | 3771 |
| hs132690462 | TGTTGTTGAGAGGATAAAAAAGG | 3772 |
| hs132687007 | ACAAATTTGAGTAAGTTATACGG | 3773 |
| hs132693787 | GCATTAAGTATCCAGCAAATTGG | 3774 |
| hs132688470 | GTTTACTACTTATTTGGTTCCGG | 3775 |
| hs132686360 | CCACTTTTCCCTAAGTCAATAGG | 3776 |
| hs132689680 | CCAAGTATCTTTCCAATTGCAGG | 3777 |
| hs132689576 | CCTTAACTTACTTAAACAATTGG | 3778 |
| hs132691377 | AGAGGTAGCTTTTCTGTCATTGG | 3779 |
| hs132693448 | CCAGGTTTTGTCAGGCACTATGG | 3780 |
| hs132689986 | TTACCATTTTTGCAGATATTGGG | 3781 |
| hs132685229 | TGTATTGTCAATCTTCCTTCTGG | 3782 |
| hs132691786 | GTAATGACTATCCCTTTAAAAGG | 3783 |
| hs132693639 | GATTTTATAAATGAGTAATATGG | 3784 |
| hs132686709 | TTATTAACCAACATAAAATATGG | 3785 |
| hs132690703 | CATACACTCTCAAAGAGATTTGG | 3786 |
| hs132692304 | TTAATTTGTAATTAGTCTATAGG | 3787 |
| hs132694041 | GTTCTATGACAAGTGTAAAAAGG | 3788 |
| hs132684210 | TGTACTTTAATGATTCTAATAGG | 3789 |
| hs132692444 | TTATGACTCCTGGACATTTTGGG | 3790 |
| hs132694247 | TATGAGAATGGGAGGATAATAGG | 3791 |
| hs132684150 | AGGTGGGCTGCCAGGTAAATTGG | 3792 |
| hs132688440 | TGGAAGTGTGAATAAAAGTTAGG | 3793 |
| hs132684670 | TCAAGTCTACCCATTCTTCTAGG | 3794 |
| hs132693447 | TATAATCTCGACGTACAATTTGG | 3795 |
| hs132692455 | AACACATTCTGCTATATCTATGG | 3796 |
| hs132691598 | AATGTGATATTTTATCAAATTGG | 3797 |
| hs132685440 | TTTAATGGCGTAAGTAAAACTGG | 3798 |
| hs132683633 | ATTCTGTCATAATTAGTCTATGG | 3799 |
| hs132688110 | CCATGGCAGGAAGATTTATTGGG | 3800 |
| hs132685317 | TTGTACTTAATAATAGTCTTAGG | 3801 |
| hs132689003 | ATAGAAACTCAGCTGAAATAAGG | 3802 |
| hs132685129 | TTTGTATCACTTAAAACATTAGG | 3803 |
| hs132693692 | GTATTAGATGTAACAAAAATTGG | 3804 |
| hs132691791 | AATCCCATTTCACTTTTGATAGG | 3805 |
| hs132689744 | GGATTGAGCTCATTTATAAAAGG | 3806 |
| hs132689425 | TTTCTGCATATATAGAAAATTGG | 3807 |
| hs132685563 | CCACTGTTCTCTATAAAATATGG | 3808 |
| hs132693462 | TTATATTGATGAACAGGTTTTGG | 3809 |
| hs132685756 | TTATATATATACATTTCTTTAGG | 3810 |
| hs132688228 | GTAGATGAATGTGATTTAAATGG | 3811 |
| hs132686157 | AGAAATCCAGTAATATTAATTGG | 3812 |
| hs132687499 | TAGTTGTCAACCTTCAAAATAGG | 3813 |
| hs132683510 | TTATTAAGAACAACTTTTGTAGG | 3814 |
| hs132687481 | GCTGAAGAATGGCAATAAAAAGG | 3815 |
| hs132693531 | TATATGGGAATCTAAATAAAAGG | 3816 |
| hs132689172 | GCTGTTATCGATTCGGTTTAGGG | 3817 |
| hs132692743 | TGTGTATTAAATGTTAAAATTGG | 3818 |
| hs132693567 | GTATCTATCTTAGGTATATATGG | 3819 |
| hs132685268 | TTAGTGAGATGAACTGTTAATGG | 3820 |
| hs132690091 | TCTTAAGCACCAACATAAAAAGG | 3821 |
| hs132684022 | ACAGTTACCCTTACTTTAGATGG | 3822 |
| hs132693438 | CCTTTCATCCCATTTGGTTATGG | 3823 |
| hs132689030 | ATTCCTTCTTTTATAACAATTGG | 3824 |
| hs132687580 | ACAATAGATGAACAAGAAAATGG | 3825 |
| hs132683728 | TCAAGCCAAAAATTATAAATAGG | 3826 |
| hs132685026 | TTATTAATGTATATGTATATAGG | 3827 |
| hs132693136 | AGACAGAGAGTAGCTCCTTTTGG | 3828 |
| hs132685209 | AGAACTTTAGGCAGAATTAAAGG | 3829 |
| hs132689915 | CTTAATTCAGAAATATATTTCGG | 3830 |
| hs132693391 | TTATCAATGCCATTAGTTACTGG | 3831 |
| hs132693636 | GATGCTGCCATATATGTAATTGG | 3832 |
| hs132684318 | ACAAAAGGATAGTTAGTAAAAGG | 3833 |
| hs132688040 | TTACTCATGTACAGAGTAAAAGG | 3834 |
| hs132691885 | GCTAAAGAACAATATATAAATGG | 3835 |
| hs132690590 | TTATAAGGCATATATTTCTTAGG | 3836 |
| hs132688561 | GATATTGCTGAGAAAAGTTTTGG | 3837 |
| hs132688554 | CCTTGAATTTATGCACTAAATGG | 3838 |
| hs132691906 | TTTGGCCTGTATATACATATTGG | 3839 |
| hs132691995 | GCCAAGTCTACAGTTCTTTAGGG | 3840 |
| hs132687892 | CTCTGTTGTGGTTTGCTTCAGGG | 3841 |
| hs132688241 | TGGCTCTTATAATAGGTTATTGG | 3842 |
| hs132685491 | CTAAATTTTCTGGCCAAATAGGG | 3843 |
| hs132685477 | CCAGTTTTCAAAAACATTGATGG | 3844 |
| hs132685890 | AGGTAAGTAAGACAGATTTTAGG | 3845 |
| hs132689835 | CTTCTAGCGCTTGTCATATAGGG | 3846 |
| hs132685993 | ATATGCTGTATTATATTTACAGG | 3847 |
| hs132683936 | GCATTGTGCATTTAAACTTTTGG | 3848 |
| hs132690779 | TGAGAGTCAGAGCCTGTTAATGG | 3849 |
| hs132683445 | ATAAAAATAACTTTATTAGAAGG | 3850 |
| hs132686884 | TGTCCAAGTCAGAAACTCTTTGG | 3851 |
| hs132690463 | TTTCACTCTACCAAATAAAAAGG | 3852 |
| hs132687095 | GCCAAGTGTCTTCCGCTAAAAGG | 3853 |
| hs132685168 | TCATATTAGCATTCACTTAGAGG | 3854 |
| hs132691015 | GGCTGTCCTTGCTAATTAATGGG | 3855 |
| hs132692286 | GATAATTCAGTGATTGTATACGG | 3856 |
| hs132686898 | TTAGACAAGCACAACTTATTAGG | 3857 |
| hs132688095 | ATACTCAGATGGACAAGTTTTGG | 3858 |
| hs132690677 | TATTTATCACACATGCAAAAAGG | 3859 |
| hs132690996 | CTATGGGCTTCCTTTCTAAAAGG | 3860 |
| hs132685710 | TTATACTGACGTTTATTATCAGG | 3861 |
| hs132689194 | GATTGTACCTTTAGACTTATGGG | 3862 |
| hs132690186 | ATAGAAATTATCCCTTAAAATGG | 3863 |
| hs132693901 | TCTTTCTCTTTCTTGTTCATTGG | 3864 |
| hs132689375 | AGATGTATACTTAAGATATATGG | 3865 |
| hs132685640 | TATGTATTTTTTTATTTAAAGGG | 3866 |
| hs132689733 | TGGACTCTGAAAACAAAAATAGG | 3867 |
| hs132694370 | GCATGAGCATGGACAGTATTTGG | 3868 |
| hs132687961 | CGTTTGGAATGACCATAAAATGG | 3869 |
| hs132686711 | ATATGTACTACGTTTTTTACTGG | 3870 |
| hs132689909 | AATCTCATACACTAGAAAAATGG | 3871 |
| hs132684722 | CCTCCTGTAACGTCAATTCTAGG | 3872 |
| hs132694092 | TAGCATGCTTAAAGCTTTAGAGG | 3873 |
| hs132686160 | ACTTAATGTACAGATTTTATAGG | 3874 |
| hs132692902 | TGTTATATAAAACCAAATAATGG | 3875 |
| hs132690895 | CTACTTTTCATGACTCTATCTGG | 3876 |
| hs132685277 | TTGAGAAACACAACTCTAAAAGG | 3877 |
| hs132690111 | CATGGAATCAGGACTATAAAAGG | 3878 |
| hs132690969 | GTAGGAATACTGGCCTTTACTGG | 3879 |
| hs132694206 | GGTAAATTCAGCAAGATTAATGG | 3880 |
| hs132693318 | CTTCTATGCTTCATTTTTGATGG | 3881 |
| hs132689673 | AAATTCCCATTGGAGAAAAATGG | 3882 |
| hs132686330 | TCTGGCCTGTGGGCCATAATTGG | 3883 |
| hs132691061 | TCCCCACTAGCCTCACCATTTGG | 3884 |
| hs132689408 | GTCCTTTTCTAAAAAAAATAAGG | 3885 |
| hs132692133 | CTAATTTTATGGTCAATAATTGG | 3886 |
| hs132690775 | ATTTTGAACCAAATTATAAATGG | 3887 |
| hs132693419 | TTTATACACAGGAATAAAAAAGG | 3888 |
| hs132686891 | GTTAAACACTTATTATTAAATGG | 3889 |
| hs132692643 | TGAATGGCTTTCCAGCCTTCTGG | 3890 |
| hs132689579 | CTTTCTGTCCAATTTATAAAAGG | 3891 |
| hs132686458 | TTTAAACTTACCCCTCAAAATGG | 3892 |
| hs132691351 | AATTTATTGGATTTAATATTGGG | 3893 |
| hs132685837 | TGTGCTTTACTATTTTCTTTAGG | 3894 |
| hs132687546 | AGTAGTGTTGTGTAAGTAAATGG | 3895 |
| hs132693049 | CTTTATCGAACCTTGTTAATTGG | 3896 |
| hs132684297 | TTGCATCTCCTTTTTCAGTATGG | 3897 |
| hs132691737 | CATCATTTACCAATATTTTAGGG | 3898 |
| hs132683388 | TTGTGACTCTGATTATCTTATGG | 3899 |
| hs132687480 | CCATTCTTCAGCATTTTTACTGG | 3900 |
| hs132691161 | TTAATATCTAACCTGTTTACAGG | 3901 |
| hs132686169 | TGAGTTATCGTTCTGTTCTTAGG | 3902 |
| hs132690550 | TCTGTGACAGTTATTAAAAATGG | 3903 |
| hs132692493 | CCAGTTACAGGCCCTTTTATGGG | 3904 |
| hs132688925 | GAGATGATCGATCTATGTTTTGG | 3905 |
| hs132687688 | TCAGCCCTCTACCCTAAATAAGG | 3906 |
| hs132689062 | GAGATCTCTCAGAAAAAAATTGG | 3907 |
| hs132693626 | GCAATAATTTAGATTGAAAAAGG | 3908 |
| hs132691735 | TTAAAGATTCCCTAAAATATTGG | 3909 |
| hs132685089 | TATTGCGGCAACTTTCTAAAAGG | 3910 |
| hs132687604 | TCACACAAGTAAAATTTTATAGG | 3911 |
| hs132684028 | CCAGACCCTCCTCTTCTAAAAGG | 3912 |
| hs132687360 | CATATAGGAGACGTAATATATGG | 3913 |
| hs132689960 | ATAAAACCCATTAAAATTTTGGG | 3914 |
| hs132692689 | ATAAAGATGTAAGTAGAATATGG | 3915 |
| hs132691688 | CCTTGGACTTGCTTCTAATATGG | 3916 |
| hs132683929 | TATAAAACAAACATATTAAAAGG | 3917 |
| hs132692592 | ATATCAATATAAAAGTTAATTGG | 3918 |
| hs132687890 | CCACAATCAGGACAGTAATTTGG | 3919 |
| hs132687177 | TCTTAAGAGCACATACTTATAGG | 3920 |
| hs132693991 | GCATGACCATGCAAACTTTCAGG | 3921 |
| hs132690137 | GTAGATGGCTTAATGGGTTTTGG | 3922 |
| hs132686326 | TTCAGTCTCTTGAGATTTACAGG | 3923 |
| hs132683595 | TTTATGCTCCATTAGCAAATTGG | 3924 |
| hs132689039 | CACACAAGTGAAAATTAAATTGG | 3925 |
| hs132689483 | CTTTGAGAGCAAACAAAAATTGG | 3926 |
| hs132689386 | ACATAGAGCTTCATACTTACTGG | 3927 |
| hs132692998 | GCAACTGCTACCACAGTTAAAGG | 3928 |
| hs132683292 | GTATTACATTCATTGATAAATGG | 3929 |
| hs132694001 | AAGTGTCACTTGAAATAAAAAGG | 3930 |
| hs132693245 | CTTTTTTTCCTTTCTGTAAAGGG | 3931 |
| hs132693741 | GCAAAAATCTGTATGCATTAAGG | 3932 |
| hs132689855 | GAAAACCAAGAGCTTATTAAAGG | 3933 |
| hs132683518 | TTTTCAGGCTCTTCATAAATAGG | 3934 |
| hs132690295 | TCTAATCTGCATTAAATAAAAGG | 3935 |
| hs132691486 | TACATACTGCAAATAATAATTGG | 3936 |
| hs132691267 | TCTTCCTTGAGTCCAGTTAAAGG | 3937 |
| hs132691189 | GGAAAAAGCTGGGAGTTAATTGG | 3938 |
| hs132686288 | TGTTCTACGTATTTCAAAAATGG | 3939 |
| hs132686710 | TGGTGTCAAGATATTAAAATTGG | 3940 |
| hs132683752 | TTATGCCACTGGATTAAAAAAGG | 3941 |
| hs132689940 | GATTTAGTATTGTAAAAAAATGG | 3942 |
| hs132684699 | TTTGAATGCAAAACACTTAGAGG | 3943 |
| hs132694215 | TCTCTAGTACTTAAGTAGTATGG | 3944 |
| hs132687841 | CTCCCTGCCATAGTTTTAAATGG | 3945 |
| hs132683334 | AATCCCTTAGTATAAATCTTTGG | 3946 |
| hs132687922 | TTTGGTGACACAAGTAAAAATGG | 3947 |
| hs132690875 | GTATTTTGCAACAGTATAATAGG | 3948 |
| hs132687535 | GTTTGGGCTTTTGTGTTAATTGG | 3949 |
| hs132688217 | CTAGGGCTAGGGCAATTAATTGG | 3950 |
| hs132689221 | GCTCGGCTATTTTTAGGTTTGGG | 3951 |
| hs132683945 | GTATTTATATTTGTCTTCTATGG | 3952 |
| hs132686003 | GCAGAGAACAACAATTTTAATGG | 3953 |
| hs132688446 | TGATGGATGTTCTTGTTTATTGG | 3954 |
| hs132683896 | TGTCATTTCTAGTACTTAATTGG | 3955 |
| hs132687957 | GTTACAGCAATGGGTCTTAATGG | 3956 |
| hs132685862 | TGGAATTCATGTATTATAAAAGG | 3957 |
| hs132687833 | ATTACCATTTCAACTCTAAATGG | 3958 |
| hs132691020 | GCAAAACGAGTGCCAATATTTGG | 3959 |
| hs132688657 | ATTGACTTCAATCTTTAATATGG | 3960 |
| hs132689333 | ACAAGGAGAACCAAATTATTTGG | 3961 |
| hs132686472 | GCACTAGAGGTTACTATAAATGG | 3962 |
| hs132684335 | TCACATTCCCTTGTAAAAAAAGG | 3963 |
| hs132689060 | AGTCAGTGCTCCTTATTTAAAGG | 3964 |
| hs132692384 | TCACAATCCCACTTTTTCTTTGG | 3965 |
| hs132688317 | TCATAAGCTAATTATTAAATTGG | 3966 |
| hs132687770 | TCATTTCAATCCACAAAAATAGG | 3967 |
| hs132687618 | ATATACACACAAGAAATTTTAGG | 3968 |
| hs132694140 | GATTGCTTCTTGCCTGAAATTGG | 3969 |
| hs132686423 | GCAGTACTCAACAAAGTAATTGG | 3970 |
| hs132691016 | AGGCTGTCCTTGCTAATTAATGG | 3971 |
| hs132685525 | GGTTATGTATATTATAAATTAGG | 3972 |
| hs132694135 | GCCGTCCTCTTACCAATTTCAGG | 3973 |
| hs132689712 | TGGCATACGGAGATCAAAATTGG | 3974 |
| hs132685552 | ATAACTTGTTTGGTGAAAAATGG | 3975 |
| hs132683911 | TTTCAATTCATGCCAATTATAGG | 3976 |
| hs132684732 | CTAACTCCAGTTTATCATTTAGG | 3977 |
| hs132693548 | TATCTGACTCTAAATAAATTTGG | 3978 |
| hs132694210 | GCAGAGACTTCAATTTTTTATGG | 3979 |
| hs132678323 | AATCAAAGATAACACCACAGGGG | 3980 |
| hs132681663 | AAGATGGTAGATGAATGCAATGG | 3981 |
| hs132678127 | GTATGAAGCAGAAACCACGAAGG | 3982 |
| hs132678114 | TATAGTATGTCCTACCACGGGGG | 3983 |
| hs132679020 | ATAGAGATCAGAAAGCCCAGTGG | 3984 |
| hs132681312 | AGGGTACTAGTGTAAGACAGGGG | 3985 |
| hs132678960 | AAGTGGAGAAAAGTTCAGAGGGG | 3986 |
| hs132678913 | TCAAAGATTCAACAACACTGTGG | 3987 |
| hs132678666 | GGGGCATCAGAAACACACAATGG | 3988 |
| hs132680468 | GAAGTTAAGAGCAAGCACTGAGG | 3989 |
| hs132681298 | GTAACATGTACCCAACAATGTGG | 3990 |
| hs132679252 | ACACTGAGAGTGGAACACAAAGG | 3991 |
| hs132677332 | ATATGCTTTGGAAAACACGGGGG | 3992 |
| hs132677361 | AGGCATGGGAAGTAACACGTAGG | 3993 |
| hs132677715 | TTCACTGTGTGTGATCCATGGGG | 3994 |
| hs132680854 | ATCTCCCTAAGCATACAGGGTGG | 3995 |
| hs132677251 | GGTGAATGCAGATGGTACAGAGG | 3996 |
| hs132678890 | GGTTAGCCCTCAAGGCAACGTGG | 3997 |
| hs132680274 | TGAATGGGAAACCATCAGAGAGG | 3998 |
| hs132683130 | TTTGTCATAGAGTATACACGAGG | 3999 |
| hs132678407 | ATGACAAAGTAAAATCACAGTGG | 4000 |
| hs132678466 | TGTACGGTCAAGAAACATGGTGG | 4001 |
| hs132682292 | TAGATATTACTCACTCAGGGAGG | 4002 |
| hs132680863 | ATTGGAGACTGTATTCACCACGG | 4003 |
| hs132678656 | AAGGGGTTGAGGGAAAGCCGAGG | 4004 |
| hs132680236 | GTAAAGGGGATAATACCCTGAGG | 4005 |
| hs132681045 | TATCATGAGAAAGACATGTGCGG | 4006 |
| hs132682883 | CACCCTTAAGTGAAGCACAGAGG | 4007 |
| hs132680658 | ACAGTGGTTTCCCTGCATGGAGG | 4008 |
| hs132678813 | GCAAACCCACTCCTACACGAAGG | 4009 |
| hs132678115 | ATATAGTATGTCCTACCACGGGG | 4010 |
| hs132680603 | GGAGAAGTGAAGAAACATGGAGG | 4011 |
| hs132681500 | ACTGAACAAAGCACTCCCGTAGG | 4012 |
| hs132682302 | TGAAATGTCTCCATTCAATGTGG | 4013 |
| hs132680837 | TGGTATTGCTAGTAGAACGGTGG | 4014 |
| hs132683067 | GATATGGGAGTTAAGATTCGGGG | 4015 |
| hs132681619 | TTTGACTGAATAGAAAGTGGTGG | 4016 |
| hs132682991 | CCTATGAGAAGCAGGCAGGGGGG | 4017 |
| hs132678425 | ACCACAACAGAGAAGGTTGGGGG | 4018 |
| hs132677450 | TCTTCTGTCAAAAGTCACAGAGG | 4019 |
| hs132679437 | ATGATGGTGAAACTGGCGGTGGG | 4020 |
| hs132678318 | TTAGAGATATTGTACCCCTGTGG | 4021 |
| hs132679242 | CATGTACATACAGTTCTACGAGG | 4022 |
| hs132677785 | TGCCTGTGTGTATGTGAACGAGG | 4023 |
| hs132678795 | AAGATAGCCTCGCAGCACAGTGG | 4024 |
| hs132679435 | ATGAATGATGGTGAAACTGGCGG | 4025 |
| hs132680592 | AAGGAAATATAAGCCCAACACGG | 4026 |
| hs132679395 | GAATTGCACACACACACACGCGG | 4027 |
| hs132681954 | GGACGCGTCAGTAAAACTGGTGG | 4028 |
| hs132681060 | TCTCTGTTATGAGTCCACAAAGG | 4029 |
| hs132679468 | GGGGCCACTTTTAAACTAGGGGG | 4030 |
| hs132682912 | AATTTATTGAACCCTCAAAGGGG | 4031 |
| hs132678361 | ATAAAACATTCAATCCCACATGG | 4032 |
| hs132679539 | CTGCAACATCAAAAACAGAGAGG | 4033 |
| hs132677945 | TCCCTTGTCCAAGAGCCCTGTGG | 4034 |
| hs132683271 | GAACTTGGTAAAGACCAAGAGGG | 4035 |
| hs132677949 | AATTCTAAATGATAACCACAGGG | 4036 |
| hs132680544 | AAAAGATGATGGAATCCCTGAGG | 4037 |
| hs132679924 | AGATACACTCGGAATTAGTGGGG | 4038 |
| hs132679119 | ATGTAACTAAGCATTTAGAGGGG | 4039 |
| hs132680979 | ATAAAGATACTAAGCCCTCGGGG | 4040 |
| hs132682746 | GTGGTGATAGTTCTAAAACAGGG | 4041 |
| hs132677368 | ATCACTATTAAAGAAGACGACGG | 4042 |
| hs132682471 | ATTAGGTATAATTATCACTGTGG | 4043 |
| hs132682690 | TCTTACATAATGTTCAAACAGGG | 4044 |
| hs132678754 | GTGATAGCCTTTGGTCAACGGGG | 4045 |
| hs132679680 | TGTATGCGTATGTCCAACAGAGG | 4046 |
| hs132679185 | AAGTGGGGTCAAGAGCCTGTGGG | 4047 |
| hs132679448 | ATACATAAAACGGAAAACTGAGG | 4048 |
| hs132677784 | CTGTGTGTATGTGAACGAGGAGG | 4049 |
| hs132678886 | TGCCTCCTATTATACCCTAGGGG | 4050 |
| hs132682364 | ATAGTGGTGTCAGATGAGAAGGG | 4051 |
| hs132682261 | ATAGCAGCCAGACAAAACAGAGG | 4052 |
| hs132680282 | TCTATCTCTTTAGGACCAGGGGG | 4053 |
| hs132680102 | TGTATTCTGTAATTGCTAGGGGG | 4054 |
| hs132682513 | CTCATGTACCTGGAAATGAGTGG | 4055 |
| hs132683156 | GTAGAAACAGAAGAAAAACTGGG | 4056 |
| hs132678090 | ATAGGCTGAAAGCTCCAACAGGG | 4057 |
| hs132680713 | TTTGAGGTACAACAGACCGAAGG | 4058 |
| hs132681473 | GCTAATGATAATCTAGACAGAGG | 4059 |
| hs132678865 | TGAGCAGAAACTGTATACAGAGG | 4060 |
| hs132680779 | TGTCTTGCTATGAAACCTAGAGG | 4061 |
| hs132677722 | AATGTGACAGATATCACATGAGG | 4062 |
| hs132680465 | TAGACGAATGGAGGTACACGAGG | 4063 |
| hs132679506 | AAAATGAACACAGACTGCAGAGG | 4064 |
| hs132682987 | AGTGAAGACTCAATAACAGGTGG | 4065 |
| hs132679519 | GAATAGGTACAGATTCAGAGTGG | 4066 |
| hs132679244 | GAGGAAATAAGAAATTCAAGCGG | 4067 |
| hs132678720 | AAGCCATTAAAACCCCTCAGAGG | 4068 |
| hs132682309 | AAGTCAGAAACACATGTGTGTGG | 4069 |
| hs132680795 | ACAGAAAGTTGGAACCAAAGGGG | 4070 |
| hs132680701 | GGAATGCATAAACACATGGGAGG | 4071 |
| hs132679823 | GTTTGGTAAATGCATCACCAAGG | 4072 |
| hs132677331 | AATATGCTTTGGAAAACACGGGG | 4073 |
| hs132679786 | GATGGGAACCAGTCTCTAGGTGG | 4074 |
| hs132678006 | TATCACCACAGGCTTCCCAGCGG | 4075 |
| hs132678647 | AGAGAAATGCTTCCTCAGTGAGG | 4076 |
| hs132682712 | CTACTGTTAAGACACAGGATGGG | 4077 |
| hs132681830 | CATGGTTTTCATGAACTGGGCGG | 4078 |
| hs132681688 | TAAACATCAGAAGACCTCAAGGG | 4079 |
| hs132679438 | ATTCATAAGATCAATGACTGTGG | 4080 |
| hs132682911 | GTTCAATAAATTCCTCAGTGGGG | 4081 |
| hs132677900 | GAGAGACCGCAAGATCAGGAGGG | 4082 |
| hs132682194 | TATGGGATCAAAGCATCCAGAGG | 4083 |
| hs132680153 | GTTTCCGTACTCCCCTAGTGGGG | 4084 |
| hs132678892 | TATAGATTAGAAAAAAATCGGGG | 4085 |
| hs132683207 | AAATATACCGAATTGCTCTGAGG | 4086 |
| hs132681337 | GCAAGAGGAAGAACACGTTGAGG | 4087 |
| hs132677073 | TAATCTCAGAAATGCCACAACGG | 4088 |
| hs132678526 | GTTTATTTTCTCTTTCTGCGAGG | 4089 |
| hs132680953 | GGGTGGGAAAGGTTTCAGAAAGG | 4090 |
| hs132680518 | GCCTTGGGAAATGAATAGGTGGG | 4091 |
| hs132680700 | AGAGGAATGCATAAACACATGGG | 4092 |
| hs132681557 | TGTGTGTGTACATATATGGGTGG | 4093 |
| hs132677134 | GCAGGGAACAATGATACCTGCGG | 4094 |
| hs132678766 | CTATGAAAACATGCACTGGGTGG | 4095 |
| hs132680614 | GGTAGGAATGCATTTCCCAGAGG | 4096 |
| hs132679415 | GGATAAACATTAGCTCTGTGTGG | 4097 |
| hs132678562 | GCACAGTTGAGACTACACCTTGG | 4098 |
| hs132677630 | AGATTAAGAGAAAAAAAACACGG | 4099 |
| hs132682986 | AAATGCTCGCTTACTCACAGAGG | 4100 |
| hs132682815 | AGCGTTCTCTATCTCCACAAAGG | 4101 |
| hs132677780 | CTCCTCGTTCACATACACACAGG | 4102 |
| hs132679359 | TCTAGATAGTATTTCCGTGGGGG | 4103 |
| hs132680512 | TTGTTACTGACTTATCTGAGAGG | 4104 |
| hs132677835 | TATCAAGGTCTCGATCGAGAGGG | 4105 |
| hs132681367 | TCATACAGAAAGGACTCAGGAGG | 4106 |
| hs132681460 | CCAAGCAAAGCACCAAACAGTGG | 4107 |
| hs132681282 | GCAAAATGTCTTAACACTAGGGG | 4108 |
| hs132677929 | CAACTAAGATAGACACAAAGGGG | 4109 |
| hs132682928 | TTTGCATGAAGCATTAGTGGTGG | 4110 |
| hs132677631 | GAACTGTTGGTGAAGCAGAGAGG | 4111 |
| hs132679526 | TCATAGAAAAGGATTCAGGAGGG | 4112 |
| hs132678672 | GTAATGTTATGAAAGAAAGGGGG | 4113 |
| hs132680792 | TTACACATAAATATGAACGATGG | 4114 |
| hs132677083 | GCTGGGAATTACCTCATGAGGGG | 4115 |
| hs132678650 | TGAGTTCTGTGGCCTCACTGAGG | 4116 |
| hs132681620 | AATCTACCTAAAATTCACAGAGG | 4117 |
| hs132682150 | AGTTGCTGTGGAAAAACCAGAGG | 4118 |
| hs132678725 | CAAATTTAATCACCTCTGAGGGG | 4119 |
| hs132677822 | ATATGCATCACTCAATACCATGG | 4120 |
| hs132679336 | AAAACAGTTAGATGGTACCAGGG | 4121 |
| hs132681708 | TCTCCTGGTAGCAAACAATGAGG | 4122 |
| hs132678678 | CAATAAGTAATACAGTATAGGGG | 4123 |
| hs132680057 | TGCACACCAGCTATACACACAGG | 4124 |
| hs132682717 | GTGACTTCATGATAAAAAGGGGG | 4125 |
| hs132677881 | CAGAATAAGAAAGCACTAGGGGG | 4126 |
| hs132679756 | TAGTGTTTGATTGAGGACGGAGG | 4127 |
| hs132682519 | TTATCTTTAGAAATGACTGGGGG | 4128 |
| hs132678418 | GAGTGTGTTCCTGAGAGGAGGGG | 4129 |
| hs132678353 | TCTAGATTTGGAGAGACAAGGGG | 4130 |
| hs132680247 | GTAAACAGGCAAAGTCACAAAGG | 4131 |
| hs132678449 | TGAAGTGCCTGGGTACTGGGTGG | 4132 |
| hs132679921 | ATATTAAGAACAGATACACTCGG | 4133 |
| hs132681939 | GTCCTTTAGAAATTTCTCTGAGG | 4134 |
| hs132679362 | GATACTCCTCATTACCCCCACGG | 4135 |
| hs132678796 | ATCACTACCACTGTGCTGCGAGG | 4136 |
| hs132681956 | AGGGTGAAGGAACCGTTTGGGGG | 4137 |
| hs132681846 | CACAGGGAACTGGCACCACGGGG | 4138 |
| hs132682275 | ATGTGGCAGTTTAATGACAGAGG | 4139 |
| hs132677440 | ACCAAGGATGACTTCCCAGTAGG | 4140 |
| hs132677857 | GTTATAGCAGTGTAGGCTGGAGG | 4141 |
| hs132677638 | TATGGAATAGCTGAGACTGAGGG | 4142 |
| hs132678587 | TTATGTGTAGATACACAGACTGG | 4143 |
| hs132677132 | ATGCCCGGGTACTTCAAGCAGGG | 4144 |
| hs132682909 | GGGTTCAATAAATTCCTCAGTGG | 4145 |
| hs132681479 | TGAGTGATAAACATCCTGCAGGG | 4146 |
| hs132677617 | TGAGGTCGAAGCTTACGGCAAGG | 4147 |
| hs132678724 | ATTTGCTGTGATAAACAGTGGGG | 4148 |
| hs132682199 | AAAACTTAAAAAGCACCTAGAGG | 4149 |
| hs132679326 | TGACTACAAAAGGTATCAGGAGG | 4150 |
| hs132677700 | TAAGTCACATCCAGAAATCGGGG | 4151 |
| hs132678719 | AGACAAAGAGTAAATTACTGAGG | 4152 |
| hs132679358 | ATCTAGATAGTATTTCCGTGGGG | 4153 |
| hs132679994 | CTACTGGGAAGGTATGCCTGTGG | 4154 |
| hs132679797 | AGAGAAGGAGGATAACTAGGAGG | 4155 |
| hs132680425 | AGAGTCCCGGTAGAAACTGGAGG | 4156 |
| hs132677195 | TGGAGGTAACCAAAATGGAGAGG | 4157 |
| hs132681311 | AAGGGTACTAGTGTAAGACAGGG | 4158 |
| hs132679143 | GCATTTTGTGCAATGCTGAGGGG | 4159 |
| hs132683053 | AGAAATGAACAAGTATCCAGAGG | 4160 |
| hs132678841 | AGGCAATACAAGAAACCTTGAGG | 4161 |
| hs132678531 | AAGGACTCAAACTTTTCGCGGGG | 4162 |
| hs132681233 | GTGAGAGTAATTGAATGCTGAGG | 4163 |
| hs132679809 | GGAGCTTTAAGAGCACTTTGGGG | 4164 |
| hs132681582 | GTAAAAAAAAATGTCAAACAAGG | 4165 |
| hs132678482 | ACAGTGCCAGGATTTGAAGGCGG | 4166 |
| hs132680237 | TAAAGGGGATAATACCCTGAGGG | 4167 |
| hs132677057 | TTGGAATATACCAGTCAGAGTGG | 4168 |
| hs132679761 | ATATATGCCAGGAGACACCTGGG | 4169 |
| hs132680008 | AATATAACTTGTTTACTGAGGGG | 4170 |
| hs132677275 | CCCTTATCAAGTGAACTGAGTGG | 4171 |
| hs132679504 | GGACTAAGCATCCCCCTGAGAGG | 4172 |
| hs132681546 | TCGAATTGCTTCAGCCCAAGGGG | 4173 |
| hs132679792 | CATGCGCATAGTCATACACATGG | 4174 |
| hs132678835 | GGACATAGTTTTCAACAGAGAGG | 4175 |
| hs132678801 | AGAGTCAGATGTCAACCGCAGGG | 4176 |
| hs132678610 | TCAAGAATTATAAGGCGGGGAGG | 4177 |
| hs132677907 | CAGGGGCTCATAAAGTAGTGAGG | 4178 |
| hs132680546 | AGTATTGTCGGATATCCTCAGGG | 4179 |
| hs132680268 | AATGAGACTTAATTACACAAAGG | 4180 |
| hs132682625 | AAGAAACCAGAAAAATCACTGGG | 4181 |
| hs132678983 | CAAAACATCTCATCAGAACACGG | 4182 |
| hs132680633 | AGACAACAGGATGTTAGCAGTGG | 4183 |
| hs132677648 | CATACTTAAGATCAAGTGGGAGG | 4184 |
| hs132678932 | AACTTCTAATAAGTAGACTGAGG | 4185 |
| hs132679363 | GGATACTCACTCCTCTGACGTGG | 4186 |
| hs132677457 | TCGCATCTTTCTGATACATGAGG | 4187 |
| hs132677959 | CTAACTTTCAGAAGATTACGTGG | 4188 |
| hs132678017 | ATTTGCCAGTAGGATGAGGAGGG | 4189 |
| hs132679505 | TGATAATGATGCTTGATGGGGGG | 4190 |
| hs132677647 | CCCCATACTTAAGATCAAGTGGG | 4191 |
| hs132679966 | TTACAGTCCAGTTACTTGAGGGG | 4192 |
| hs132681790 | AGGAAAGCCCTGTAATGACCTGG | 4193 |
| hs132681488 | AACACTACCCATGATTACACAGG | 4194 |
| hs132680072 | TATGGCCAATTAGTTGAACATGG | 4195 |
| hs132680982 | TAAAAGCACAAGAGGCCCCGAGG | 4196 |
| hs132680188 | GTTTAAACTACACTATAATGGGG | 4197 |
| hs132683180 | ACTAATCTGTGTATCCTACAGGG | 4198 |
| hs132678288 | ATTCCAAGTACAGACAAGTGTGG | 4199 |
| hs132677565 | TAAACACTAAGTCTTACGAAAGG | 4200 |
| hs132677056 | TAAACTTTATGTACCCACCCAGG | 4201 |
| hs132682463 | TTACTGGACTTAAAAGAACATGG | 4202 |
| hs132677973 | TAATCATGAAAAGAGTGGTGAGG | 4203 |
| hs132677783 | TGTGTGTATGTGAACGAGGAGGG | 4204 |
| hs132682233 | ACATCAACAGTAAAGCTAGACGG | 4205 |
| hs132677629 | GATTAAGAGAAAAAAAACACGGG | 4206 |
| hs132681976 | CTATATAATTTATATCATGGGGG | 4207 |
| hs132680290 | AGGCACACCACTAGAACACGGGG | 4208 |
| hs132682448 | TTTGGTACGCTTTCAAACTGGGG | 4209 |
| hs132677882 | ACAGAATAAGAAAGCACTAGGGG | 4210 |
| hs132682002 | ATTTGAAACAACAAGCAAGGAGG | 4211 |
| hs132677683 | GTATGAAAAGGACACGAGAGAGG | 4212 |
| hs132677289 | CAGATAGGTAGGTTGACAAGTGG | 4213 |
| hs132682316 | GATCCTTTCCATAGACTCTGAGG | 4214 |
| hs132678912 | ACATTATTATCACAGCTGCAAGG | 4215 |
| hs132679096 | ATTGGGATACTCCTATGCCAGGG | 4216 |
| hs132678068 | GATGCGGGAAAGGTGGACTGGGG | 4217 |
| hs132681427 | GCAAAAAGCACAGTGTCAAGGGG | 4218 |
| hs132681104 | TAAGGGCGTAGGGATAGGAAAGG | 4219 |
| hs132681667 | AATATGTAGACATATTGGCAGGG | 4220 |
| hs132679000 | TTTTGTTTTAGATATTAGAGAGG | 4221 |
| hs132681588 | GGTTACATCTAACCTCACAAAGG | 4222 |
| hs132680945 | AGAATATGTCAGAATTCCTGGGG | 4223 |
| hs132677660 | TAGCTTCCAAGAATGAGGGAAGG | 4224 |
| hs132680396 | ATCTGAGGATGTGCTTGCAGTGG | 4225 |
| hs132677338 | TTGGCCTCAAAGTAACAGAAGGG | 4226 |
| hs132679371 | ACGCATTTCTACAAAAACCGTGG | 4227 |
| hs132679313 | TCACATGAATAAAGTTCCTGAGG | 4228 |
| hs132679115 | TGTATGGTGTCTATATACGTGGG | 4229 |
| hs132678271 | ATAATGATTTCCAATCAGGTAGG | 4230 |
| hs132682438 | CACCTGGAGAGTTTACTGAAAGG | 4231 |
| hs132679057 | ATATTTAGCACATGACTAGGTGG | 4232 |
| hs132679472 | TTCAAGAAAAATGTTACCAGGGG | 4233 |
| hs132678713 | GCTTATGTTAAGGAAGCATGGGG | 4234 |
| hs132682842 | GCTAGCGAGCTAAACCAAAGAGG | 4235 |
| hs132678067 | GGTGGACTGGGGATACAATGTGG | 4236 |
| hs132677311 | CACAAAGAATTGCAAAACTGGGG | 4237 |
| hs132678962 | GAAAGTGGAGAAAAGTTCAGAGG | 4238 |
| hs132682357 | AACTTTATAGTACCCTACCCTGG | 4239 |
| hs132680214 | GATGGTTTTGAGTACTCAGGAGG | 4240 |
| hs132680349 | TTAAATGTAAATAATCCGTGGGG | 4241 |
| hs132681768 | ATGGCCTTTTGGCATCTGAGAGG | 4242 |
| hs132682496 | GAAATGGCATATTAAAACAGAGG | 4243 |
| hs132679822 | CCTTCATATCTGTAATCATGAGG | 4244 |
| hs132678659 | GAAACTGTAACAGTTGTAAGGGG | 4245 |
| hs132677052 | AAGACCACAATCTACCTGGGTGG | 4246 |
| hs132680871 | GCAGTGTATGAAGATTCAGAAGG | 4247 |
| hs132678981 | TTATTAATTAAAGACTTACGTGG | 4248 |
| hs132680430 | GGATTGATTTCTCAATCTCGGGG | 4249 |
| hs132678307 | GATTACGTGTGAGTGGTGCTAGG | 4250 |
| hs132680694 | GAGGATTCTCAGTTAAGGGGAGG | 4251 |
| hs132679825 | TTACACATAAGCAGAAGGAGAGG | 4252 |
| hs132682632 | ATTAGCCTGTAATTTTACAGCGG | 4253 |
| hs132680891 | GTAAAGTGGCAAATAGTTGGGGG | 4254 |
| hs132677718 | GTTTCAAGAACAGTATACTGTGG | 4255 |
| hs132680462 | TTGGATGAGTAGACGAATGGAGG | 4256 |
| hs132679396 | ACGCGGCACAATAAAACCCAAGG | 4257 |
| hs132683073 | AATGAGCTTCCTCAAACACAGGG | 4258 |
| hs132681537 | AGGTAACTGAAAACTTTCAGTGG | 4259 |
| hs132678364 | ATTCCAGGAAAGCTGCCATGTGG | 4260 |
| hs132681069 | TCAAGGACCTCAGAGTCGAGTGG | 4261 |
| hs132679501 | ACGAGATTCTTTCCTCTCAGGGG | 4262 |
| hs132681841 | TTGGGCCAGTGTAGGCCCCGTGG | 4263 |
| hs132681618 | GAATTTGACTGAATAGAAAGTGG | 4264 |
| hs132679907 | CTTTGCCAATGAAATGTGTGTGG | 4265 |
| hs132679238 | TTTCGTATGAAACCTCACATTGG | 4266 |
| hs132677685 | AGAGAGGACATGATATCCAAGGG | 4267 |
| hs132678056 | GTATGGGGAGAGTACAGGTGTGG | 4268 |
| hs132680802 | GGGAAACACTAACACAATGGTGG | 4269 |
| hs132680278 | ATTACAGACTAAATACCCCCTGG | 4270 |
| hs132680061 | TCAAAAAACGTAGAACGGAAGGG | 4271 |
| hs132679066 | CTGAACATGTGTAAAGCAAGGGG | 4272 |
| hs132677567 | TAGTTGCATTTACAATACAGTGG | 4273 |
| hs132678799 | GTGTACGTGTGTGTATGCATGGG | 4274 |
| hs132680558 | GAAGTATTAGCGAAGTGACAGGG | 4275 |
| hs132682789 | ACCTGGTGCAGAGGTCCCTGAGG | 4276 |
| hs132682133 | ATGTATGCATGTGTTATGGTAGG | 4277 |
| hs132679172 | AATGTGCTGGATGAATAAGAGGG | 4278 |
| hs132679169 | ATATAGCAAATATTCAACAATGG | 4279 |
| hs132678412 | ATAGTATCTAGGCTCAGAGACGG | 4280 |
| hs132681292 | ACACCTCCACACATATGGGAAGG | 4281 |
| hs132682477 | TTTTTTTCTACATAAAAACGTGG | 4282 |
| hs132678964 | CATTACGTTTCCATTTGCTGCGG | 4283 |
| hs132682085 | AAGTGATCACCAGAGACACATGG | 4284 |
| hs132677259 | GTGTATGTTGAAGATGAAAGGGG | 4285 |
| hs132680358 | AAAAACTGTCTAGAAACCACAGG | 4286 |
| hs132681754 | ATTGGAAATCAACTGCATGAGGG | 4287 |
| hs132681457 | TACATCCATGAAGCAGCAAGGGG | 4288 |
| hs132679810 | GAGCTTTAAGAGCACTTTGGGGG | 4289 |
| hs132677124 | TTTAGACTATTCCTACAACCAGG | 4290 |
| hs132679670 | CTACTGCCTATCTCTACCAAAGG | 4291 |
| hs132678265 | GGCCTGCTGTATTCCCTGGGTGG | 4292 |
| hs132677223 | AAAAAACCACATCAGACCGTGGG | 4293 |
| hs132683270 | TGAACTTGGTAAAGACCAAGAGG | 4294 |
| hs132682297 | AAAAATGATTAACAAAACACAGG | 4295 |
| hs132680486 | AGAGAACCAGAATGATTTTGGGG | 4296 |
| hs132678507 | ACATAAGGATCTACATGCAGTGG | 4297 |
| hs132682441 | GTAAACTCTCCAGGTGAGAGGGG | 4298 |
| hs132678845 | CTTAGCAAGAAGAATTGGAGTGG | 4299 |
| hs132677300 | AGTAGAAAGTAAAGAACACAGGG | 4300 |
| hs132678487 | GACAAAACCCCAGAGAAAGGGGG | 4301 |
| hs132680222 | AAATAGGTAGTGCCTTACAAAGG | 4302 |
| hs132678324 | AAATCAAAGATAACACCACAGGG | 4303 |
| hs132683017 | TCCTCACTATTTCTGAAACGAGG | 4304 |
| hs132681414 | AAGATTCAGCTACATCTGGTGGG | 4305 |
| hs132681788 | TGGATCACCAGGTCATTACAGGG | 4306 |
| hs132679824 | AAGGCTTACACATAAGCAGAAGG | 4307 |
| hs132678277 | TGAGTGACAGTTAAGTGGCAGGG | 4308 |
| hs132682588 | TGACAGGCATGATAGAAGCAGGG | 4309 |
| hs132682596 | GAACAGCCTGCATATTGCCACGG | 4310 |
| hs132682109 | TCTTTCAATTAAATATCGGGAGG | 4311 |
| hs132678064 | ACATTCATCTCAATTGAGCAAGG | 4312 |
| hs132679043 | GATAAACAGCATTTATACTGTGG | 4313 |
| hs132677880 | AAAGCACTAGGGGGCAACTGTGG | 4314 |
| hs132680740 | TACATGGCATCCAGAGAACAGGG | 4315 |
| hs132682283 | GACAGCCTCTACAAGTAGCAGGG | 4316 |
| hs132681370 | TCAGGAGGTCAATGTAAAGGAGG | 4317 |
| hs132681288 | AACTCTATCCAGGAGACATGGGG | 4318 |
| hs132682006 | AAGGAAAATGACAGAAATAGCGG | 4319 |
| hs132679621 | AATTTGTGTGTGATGTGTGGTGG | 4320 |
| hs132678589 | TCAATCCCCTCATATACAGAGGG | 4321 |
| hs132677763 | TGAGCAAAGCAGGAAAGGGTCGG | 4322 |
| hs132680549 | CATGTGCCTAGCACAAATAGAGG | 4323 |
| hs132677465 | TTGACTATCATCACATTCAGGGG | 4324 |
| hs132677370 | CCTTAGCAGATAAAATCAGTAGG | 4325 |
| hs132682647 | GTTGTGCTCATAGGGCACAATGG | 4326 |
| hs132681363 | CTTCTCTTGTAGATCACCAGTGG | 4327 |
| hs132677344 | GGTGATAACATTTAACAGGAGGG | 4328 |
| hs132681325 | GCTAATATATATAATTAAGGAGG | 4329 |
| hs132678664 | GTAATGTTGCCAGTACTGGAAGG | 4330 |
| hs132679548 | TTTACTATCCCAAAGTTGGGGGG | 4331 |
| hs132681248 | GTTCTTGCTATCATTAAGAGGGG | 4332 |
| hs132681697 | AAAGACAGGATATATAAGCAGGG | 4333 |
| hs132683262 | TGACTCTAGATGATTATAGGTGG | 4334 |
| hs132678065 | TGAATGTGTTCAAAAAAACCAGG | 4335 |
| hs132681141 | TAGGTAAATTAAATAATGGGAGG | 4336 |
| hs132679826 | TACACATAAGCAGAAGGAGAGGG | 4337 |
| hs132677924 | TAAAGTTTAAATGTACCTAGAGG | 4338 |
| hs132680324 | GAGTGTCTCCAGTGCCAGAGAGG | 4339 |
| hs132681195 | GTTGTCCCAGTAGAATCAGTAGG | 4340 |
| hs132677051 | CAAAAGACCACAATCTACCTGGG | 4341 |
| hs132679534 | AGATAAATATCGGAATCTGTGGG | 4342 |
| hs132680068 | ATTTGCAGTAGAGGAAACAAAGG | 4343 |
| hs132678250 | AGGAACTTAAAGTTCCCACTAGG | 4344 |
| hs132679436 | AATGATGGTGAAACTGGCGGTGG | 4345 |
| hs132678041 | TTCCCTCTAACAAACAGCCATGG | 4346 |
| hs132677943 | CATATACATTTCCCCAACCAGGG | 4347 |
| hs132678591 | ACCTGCCCTCTGTATATGAGGGG | 4348 |
| hs132679085 | TATAGTTCCAGCAGAAAGCGAGG | 4349 |
| hs132679547 | GTTTACTATCCCAAAGTTGGGGG | 4350 |
| hs132677421 | TTAGGCTTCAGATCTAGTAGAGG | 4351 |
| hs132677233 | AGTATGTTAGATTTTACCTGGGG | 4352 |
| hs132679604 | ATGGTATCACCTACTGTCAGAGG | 4353 |
| hs132680579 | CAAAAGGTAAGAGAATAGGATGG | 4354 |
| hs132681307 | GCTTTCATACAATACCCTGAAGG | 4355 |
| hs132682549 | GACACAATTATAAAACCAAGAGG | 4356 |
| hs132681210 | ATGTTGGAATCTACAGACCATGG | 4357 |
| hs132680331 | AGTATTACCGGTTGTCTAAGGGG | 4358 |
| hs132681064 | ATTCCAATACGATTGCTCCAAGG | 4359 |
| hs132677739 | AGATTTACCTGATGGTGAGGTGG | 4360 |
| hs132681511 | GGATGAAAGAGGAGACAAAGGGG | 4361 |
| hs132680870 | CAGTGTATGAAGATTCAGAAGGG | 4362 |
| hs132680337 | TATCCATTAACAGCTAAGGAAGG | 4363 |
| hs132681400 | GAAGCCAAGATGAGGTAAAGGGG | 4364 |
| hs132681586 | AAATTCTGTCTAATTTCACATGG | 4365 |
| hs132679690 | GTAGGACAGTCAAAGAGTGGAGG | 4366 |
| hs132677177 | CATATAGCATCCAAGACACAAGG | 4367 |
| hs132677067 | ATAGATGAAAAGCTCTCACAGGG | 4368 |
| hs132677574 | TTATAAGCTATAATTTCCAGTGG | 4369 |
| hs132680477 | GTAGTGAAGTTGCTTATCTGAGG | 4370 |
| hs132681923 | TCAATAAAACCACAAACACCAGG | 4371 |
| hs132677891 | AAAGGAATGGCAAGGCACATGGG | 4372 |
| hs132677168 | TTATGAAAAGAAAAAATCCAGGG | 4373 |
| hs132677655 | ACTTGATCTTAAGTATGGGGAGG | 4374 |
| hs132679443 | TGTTATGGCTAGGATAACTTGGG | 4375 |
| hs132681304 | TATTACTGCAGTACAAGCCAGGG | 4376 |
| hs132679240 | AATCTATAGTGAGATATGAAGGG | 4377 |
| hs132678785 | TTAATTCTCATGGTCATGCGGGG | 4378 |
| hs132679404 | GTGAACATCCACATGTACCTGGG | 4379 |
| hs132681158 | AGATTACCTTTCTGGTAGGGAGG | 4380 |
| hs132683195 | ATAAAAGTTTACAAAGACAGAGG | 4381 |
| hs132678980 | TTAATTAAAGACTTACGTGGAGG | 4382 |
| hs132677832 | GGCAGAGCCTCCATTCCAGAGGG | 4383 |
| hs132678130 | ATACCAGAGTAAGTACAGCTAGG | 4384 |
| hs132678967 | AGTATTCTGCCAAAATAAAGGGG | 4385 |
| hs132682157 | GCTGCCAACACCACAGTTGGGGG | 4386 |
| hs132681351 | ACATTTACTAGGCGTTAACAAGG | 4387 |
| hs132678109 | TGCTCCCATGCATACCCCCGTGG | 4388 |
| hs132680448 | CTCAGGCTTTGGAAACAGGGGGG | 4389 |
| hs132681403 | GTTACTTTGAAGCCAAGATGAGG | 4390 |
| hs132679019 | AAGAAAGTTTGTGAACCACTGGG | 4391 |
| hs132678177 | AATATTGTCAAAAATCCACCTGG | 4392 |
| hs132679583 | ACTTCTAGTATTCAGAAGAGAGG | 4393 |
| hs132682178 | TAAAGTATAGAAGATGTCCAAGG | 4394 |
| hs132681191 | TACAACCTACTGATTCTACTGGG | 4395 |
| hs132681845 | CCTGTGCTAAGAGGCAGCCAGGG | 4396 |
| hs132682060 | TGTCATGTACAGTGCTAGGTTGG | 4397 |
| hs132678777 | TTGCTAGGCTTGGCTTTCAGCGG | 4398 |
| hs132683043 | AGGATGATACTGGGACAAAGTGG | 4399 |
| hs132677245 | GTGGGAGTGCAAGTCAATAGGGG | 4400 |
| hs132681951 | GGTGGTAGAGACTAGGCTGAAGG | 4401 |
| hs132682938 | TGAAGAATTATATTGTTGTGGGG | 4402 |
| hs132677746 | AAGGGAACTAAAAGAATCGGAGG | 4403 |
| hs132678693 | CATCATCCTGCATCATGTGTGGG | 4404 |
| hs132677246 | GAGTGCAAGTCAATAGGGGAAGG | 4405 |
| hs132678117 | CTATATAGTATGTCCTACCACGG | 4406 |
| hs132680538 | AAGTAATAAGACATTTCCTAGGG | 4407 |
| hs132677103 | ACTAAAGAACATGTAACAGTGGG | 4408 |
| hs132680071 | CTAATTGGCCATAGTTACTGAGG | 4409 |
| hs132681945 | AAGGGAAGAAGACATTGTGTCGG | 4410 |
| hs132683021 | ATATTGATACAGGAAACTTACGG | 4411 |
| hs132677866 | AAACATATCTCCCTACAGAAGGG | 4412 |
| hs132682440 | TTTACTGAAAGGCATACCTAGGG | 4413 |
| hs132678852 | GAACATTTTCTAATTGAGGGTGG | 4414 |
| hs132678415 | AACAAGAGTGTGTTCCTGAGAGG | 4415 |
| hs132679493 | TCAAAGAAGACCATAGCACTAGG | 4416 |
| hs132679067 | GTAAAGCAAGGGGTCAGTTGCGG | 4417 |
| hs132677483 | GTTATATCATTTGACATCGGGGG | 4418 |
| hs132682709 | ATCACCTACTGTTAAGACACAGG | 4419 |
| hs132680951 | TTCAGAAAGGAGAAACCCATGGG | 4420 |
| hs132683070 | GAGTTAAGATTCGGGGACAGTGG | 4421 |
| hs132679568 | TGAGCATCTACACTGTGTCATGG | 4422 |
| hs132681832 | GAACTGGGCGGACTGCTACAGGG | 4423 |
| hs132680958 | AGTATATTTCTGAGGACTGGGGG | 4424 |
| hs132677922 | TTAATGTTTTCATTGAACGCAGG | 4425 |
| hs132678021 | TTCATAAAATGGGAACAAAGTGG | 4426 |
| hs132680433 | TCTCAATCTCGGGGTATGTGAGG | 4427 |
| hs132680526 | GCCTAATTCTACAGTAACTGGGG | 4428 |
| hs132681789 | CATCTTCTTTAGAACCAAGTCGG | 4429 |
| hs132680654 | TCCAGCATATCCTCCATGCAGGG | 4430 |
| hs132677936 | GCATCAGAATAATTGCTAGAGGG | 4431 |
| hs132679566 | TCTGGCCTTAAGTTCAAAAGGGG | 4432 |
| hs132678605 | GAAAATCAAGAATTATAAGGCGG | 4433 |
| hs132677539 | ATTGCATTGGAGGAACTGCATGG | 4434 |
| hs132677247 | TATTGACTTGCACTCCCACAGGG | 4435 |
| hs132679888 | ATGCTGGCATGAGGAGAGTGAGG | 4436 |
| hs132680224 | CTAATCAAATACAAGAAACAGGG | 4437 |
| hs132677181 | TGGATGCTATATGATCCTGATGG | 4438 |
| hs132680799 | ACACAATGGTGGCATGATAGGGG | 4439 |
| hs132678755 | GCTATCACATTTCCCACCATAGG | 4440 |
| hs132678643 | GCCTTCATTTCTGTGTCACGTGG | 4441 |
| hs132677354 | AGCCAAGGAGATCAATGCCAGGG | 4442 |
| hs132679350 | AACCTTCTAGTCAAACTCCAGGG | 4443 |
| hs132682168 | AGAACTGCTTTGTCCCCCAAAGG | 4444 |
| hs132678258 | GTCACCCTACTATTTCCTAGTGG | 4445 |
| hs132677345 | AGGTGATAACATTTAACAGGAGG | 4446 |
| hs132679083 | ATGTCCTGTAAATGTTCGAGTGG | 4447 |
| hs132680161 | AAATCCACCCACCCCACTAGGGG | 4448 |
| hs132678821 | TCAAGGTTGATAAGGGTCAAGGG | 4449 |
| hs132683071 | CAATCAGTCAGATTAAAGAGAGG | 4450 |
| hs132677229 | CCTGGGGTATTCATACTACAGGG | 4451 |
| hs132680657 | GTATACACCAATACTTATGAGGG | 4452 |
| hs132681053 | GCAAACAATAAGAAGCAGATGGG | 4453 |
| hs132681109 | AGATCTACATCAGCAGCCAAAGG | 4454 |
| hs132681114 | GACATAATGTCTGATGGCATAGG | 4455 |
| hs132677488 | CTGAGCACACTGGAGTCCTGAGG | 4456 |
| hs132682602 | AGGATCCCAGCCTTCTTCCGTGG | 4457 |
| hs132677824 | TCACTCAATACCATGGTAGGAGG | 4458 |
| hs132682562 | CTTGGCATTTCGGGAAACAGCGG | 4459 |
| hs132679258 | GCAAATTTCAGGTCTCTGAGGGG | 4460 |
| hs132679820 | CTCATGATTACAGATATGAAGGG | 4461 |
| hs132681806 | ATTCCACTAAACATAACGATGGG | 4462 |
| hs132683147 | TATTTTGTAAAGCTACGGAAGGG | 4463 |
| hs132677399 | TTATAGGGGATATTTAAAGGTGG | 4464 |
| hs132679866 | TTAACCTCATCCTCATAATGGGG | 4465 |
| hs132678495 | AGATGCCCCCCCTTTCTCTGGGG | 4466 |
| hs132677224 | AAAAAAACCACATCAGACCGTGG | 4467 |
| hs132680943 | AACCTGCTGCAGAACACCCTAGG | 4468 |
| hs132683164 | AGGCAGTAATCTGAGCTAGAGGG | 4469 |
| hs132679985 | GATTAACTTTTGGATATTGGGGG | 4470 |
| hs132677521 | TTTGCCTAAGCAGCTTTGGGTGG | 4471 |
| hs132681501 | GCACTCCCGTAGGTACTGAAAGG | 4472 |
| hs132678489 | CAAAACCCCAGAGAAAGGGGGGG | 4473 |
| hs132678749 | TGGCATATCTAGCCTATGGTGGG | 4474 |
| hs132677972 | ATCTCTAATCATGAAAAGAGTGG | 4475 |
| hs132679369 | TCTGCAACATGCTGAATCCACGG | 4476 |
| hs132678308 | AATAAATGATTACGTGTGAGTGG | 4477 |
| hs132678203 | GTATGACTAAGAGTGTGGATTGG | 4478 |
| hs132682018 | GGGCAAATTTCAATTTTATGCGG | 4479 |
| hs132680981 | AAAAGCACAAGAGGCCCCGAGGG | 4480 |
| hs132681566 | CTATAAAGCTATAAAATGAGTGG | 4481 |
| hs132678772 | TCACGTCCAGAGGCACACACTGG | 4482 |
| hs132680561 | AAGTTGGAAGAAATATTGGAAGG | 4483 |
| hs132681294 | CTTCAACCTTCCCATATGTGTGG | 4484 |
| hs132680369 | TCAGGAGTAGTAGTAACTCATGG | 4485 |
| hs132679906 | ACATTACAAACTCTGATGAGTGG | 4486 |
| hs132680829 | CATCCAGTGTAAAGGTGCAGAGG | 4487 |
| hs132681361 | ATCACCAGTGGGCATTCCTGAGG | 4488 |
| hs132678204 | AATCTGTATGACTAAGAGTGTGG | 4489 |
| hs132681771 | GATGCCAAAAGGCCATGACTGGG | 4490 |
| hs132678688 | CCTTGCTTACACGATCATTGTGG | 4491 |
| hs132680362 | CTCTTAATCCCATCATAAAGGGG | 4492 |
| hs132680880 | CAGAAGGACCGGAGACAGGTAGG | 4493 |
| hs132682538 | AATCCCCAACTACAATGCGTTGG | 4494 |
| hs132677227 | GAATAAAACTGAGCACCGAAAGG | 4495 |
| hs132680321 | TCTCCAGTGCCAGAGAGGGAGGG | 4496 |
| hs132682807 | TATGCACATATTATATAGTGAGG | 4497 |
| hs132682962 | GTCAAGATCTCAGTAACACTGGG | 4498 |
| hs132678756 | GGGTGTTAAGAAAACATGACAGG | 4499 |
| hs132677415 | GAAACGGTATGGTACAAGGTTGG | 4500 |
| hs132679217 | ACAGAAGTGTCAAAGTTGAGAGG | 4501 |
| hs132678464 | TGACCGTACAGAGATATGAAAGG | 4502 |
| hs132682716 | TGTGACTTCATGATAAAAAGGGG | 4503 |
| hs132680883 | AATTTTGTCCAATTCGCAGGTGG | 4504 |
| hs132683235 | TGGAGCCAAAACAATCCACTAGG | 4505 |
| hs132683143 | CAAATGACGCCACAACAACAAGG | 4506 |
| hs132680048 | TTTACCCATAACGTAAAGGAAGG | 4507 |
| hs132678542 | TAGATATTAAGGCCTGTGGGGGG | 4508 |
| hs132678717 | TAAATTACTGAGGAGGAAGACGG | 4509 |
| hs132680134 | AAGCTCAGTCTACTTTATCAGGG | 4510 |
| hs132678178 | TGGTATAGCAATTTTCCAGGTGG | 4511 |
| hs132680469 | TTAACTTCCATAATCAAAGGAGG | 4512 |
| hs132682609 | TTGTATGGTAGTTACAACATTGG | 4513 |
| hs132677820 | TCAGTGACTCTAGCTGAGAAGGG | 4514 |
| hs132678607 | AAATCAAGAATTATAAGGCGGGG | 4515 |
| hs132677071 | GAGACACACTATTAAATGAGTGG | 4516 |
| hs132680661 | ATAAGTATTGGTGTATACAGTGG | 4517 |
| hs132679489 | ATGAAACATTCAGTAGCAGTGGG | 4518 |
| hs132682873 | TTTCTTGTAATGCACATGGAGGG | 4519 |
| hs132678257 | TCACCCTACTATTTCCTAGTGGG | 4520 |
| hs132682747 | ACCACAGTCACTGAAGCCCTGGG | 4521 |
| hs132678370 | AGGATTTTGGTTATACCAAGTGG | 4522 |
| hs132682041 | AACAAATCCTAATGAACCCAGGG | 4523 |
| hs132680184 | GGTGAAGAAAGGAGAGTCGTGGG | 4524 |
| hs132680784 | GGCAAGCTTGCCTAAAAGTGAGG | 4525 |
| hs132681999 | TTGTGGAGAGAACTACGCTAGGG | 4526 |
| hs132678213 | AATCAATTTCAGGCACTGAACGG | 4527 |
| hs132679361 | GGAGTATCTCGCCACGTCAGAGG | 4528 |
| hs132677986 | GAGAGGCATAGGGAGAATAACGG | 4529 |
| hs132682551 | AAAAACCTAACTTTAGAACCCGG | 4530 |
| hs132678581 | GAACGTTTTATCATGATCTGGGG | 4531 |
| hs132678814 | TTACTGTGCTAATCCACACCTGG | 4532 |
| hs132679486 | GTTATAATCCACATGATACCAGG | 4533 |
| hs132678262 | CTATACAAACATACCACCCAGGG | 4534 |
| hs132679945 | TCACATACTGAGAACTGCATAGG | 4535 |
| hs132677376 | TTGAACACTTGCTAGCTGCAAGG | 4536 |
| hs132680288 | GCAGGCACACCACTAGAACACGG | 4537 |
| hs132677597 | GATAATGATATTTTAAACCAAGG | 4538 |
| hs132678083 | GTTTGACATGACCGTAATGGAGG | 4539 |
| hs132681928 | GTACACTATTTGTCAACCAAAGG | 4540 |
| hs132681665 | ACATGCCCTTTATGGCACAATGG | 4541 |
| hs132682801 | TTAGCTTACCTATAACAAAGTGG | 4542 |
| hs132682255 | ATTCACACATCAATCAGAAGTGG | 4543 |
| hs132681523 | TACACTATAAGATAAATGTAGGG | 4544 |
| hs132682888 | GAGAGATTGAAAGACTCTGTAGG | 4545 |
| hs132680857 | ACTCTGTTGGCACATTGCAGGGG | 4546 |
| hs132677430 | CCATACACAACTGCACCGTAGGG | 4547 |
| hs132680587 | AATTTTCTGTAAAGTAGCAGAGG | 4548 |
| hs132678316 | AATTTAATCACAGCAGTCAAGGG | 4549 |
| hs132678635 | CCTCTCGCGTCATAGTTTGGGGG | 4550 |
| hs132679806 | ACATAGTACAAAATTCATAGAGG | 4551 |
| hs132681490 | ACACAGGTTCATAATGCCACAGG | 4552 |
| hs132681094 | AGACAAATCTAGAAACAACTAGG | 4553 |
| hs132681348 | AATGTGTTACAGTAAGGAGGAGG | 4554 |
| hs132682916 | TCTGTAGTAGAGAATGTTGGAGG | 4555 |
| hs132682257 | ACTGGAATCCAGGAGCCAGATGG | 4556 |
| hs132680431 | TCAATCCTCCAGTTTCTACCGGG | 4557 |
| hs132677343 | TAAATGTTATCACCTGGAGGTGG | 4558 |
| hs132682092 | TAGAATTTGTAGGAGTAAGGAGG | 4559 |
| hs132681433 | ATGAGGCCTAAGATTTGGAGTGG | 4560 |
| hs132682759 | TTTTGAAGAGAAAATGTGGGTGG | 4561 |
| hs132678016 | GGATGAGGAGGGCATAGCAGAGG | 4562 |
| hs132677714 | CTGTGTGTGATCCATGGGGTAGG | 4563 |
| hs132681438 | ACAGCCATCAAGGTGTACTTGGG | 4564 |
| hs132682173 | GTATTTGCTTTTCATTGTGGGGG | 4565 |
| hs132682917 | AGGTCTGTAGTAGAGAATGTTGG | 4566 |
| hs132678049 | GGAGAAGAGGAAGGTCTGAAAGG | 4567 |
| hs132677966 | TCAAACCTGTAGCTCTATGGAGG | 4568 |
| hs132680836 | GACTGGTATTGCTAGTAGAACGG | 4569 |
| hs132678518 | GAAGCTGTTGAAGAGCCTTGTGG | 4570 |
| hs132682017 | TCAGCTAAGCATGCTAACCTCGG | 4571 |
| hs132679463 | GATCAGAGGCTTCAGTAGGGAGG | 4572 |
| hs132680780 | TTCATAGCAAGACAATAGATGGG | 4573 |
| hs132678358 | TCAAGCTGAATACAATTCAGTGG | 4574 |
| hs132680255 | TAATCACCTACCCCGACCAATGG | 4575 |
| hs132678132 | TCAAAGATGTAGACTGACACTGG | 4576 |
| hs132679243 | GTAGAACTGTATGTACATGAAGG | 4577 |
| hs132679147 | CATGTAGCTAAGGTTGCTAACGG | 4578 |
| hs132680814 | CCTTGGGATGGGAAACACAATGG | 4579 |
| hs132681308 | CTTTCATACAATACCCTGAAGGG | 4580 |
| hs132677409 | CTAATTACAATCTATCCCAATGG | 4581 |
| hs132677135 | GTTCCCTGCTTGAAGTACCCGGG | 4582 |
| hs132681689 | GTAAACATCAGAAGACCTCAAGG | 4583 |
| hs132680052 | ACACAGCTGATATAATTTGGTGG | 4584 |
| hs132677841 | ACATAGGCAATAGTAAATCAGGG | 4585 |
| hs132678460 | TTGAGCACTGTGTTTGCTGGAGG | 4586 |
| hs132681251 | AGTATAATATGTGCAAAACTGGG | 4587 |
| hs132680128 | TGTTACTAGAACTAAAATGAGGG | 4588 |
| hs132679580 | TTTGACTGGGCTATTCTGGGAGG | 4589 |
| hs132679714 | GCATGTCTAAGAACATCCCTAGG | 4590 |
| hs132683038 | CGAGCAGAGCTATTGTGCCATGG | 4591 |
| hs132680834 | TCTATTGGAGGAAATACAGATGG | 4592 |
| hs132679497 | AGGTAATTGGGAAATCCAGTTGG | 4593 |
| hs132682470 | GTGGTAAGCAGTCTCCAGAATGG | 4594 |
| hs132678869 | ATAAAGGGTTCAAAAATCTGTGG | 4595 |
| hs132680089 | TACAATTTCCTCAAAAACTGGGG | 4596 |
| hs132678686 | CCACAATGATCGTGTAAGCAAGG | 4597 |
| hs132681106 | GAGAACACTTTTTAGAGCCAGGG | 4598 |
| hs132677915 | AGCTCCAAATTTAAGAAACACGG | 4599 |
| hs132681850 | CCTGGCTGCCTCTTAGCACAGGG | 4600 |
| hs132680952 | TTTCAGAAAGGAGAAACCCATGG | 4601 |
| hs132681899 | AACACTTTGGTAACAACAGTAGG | 4602 |
| hs132681805 | CATTCCACTAAACATAACGATGG | 4603 |
| hs132681360 | GTGATCTACAAGAGAAGTTGGGG | 4604 |
| hs132682026 | CCTAGTCTTGCAAATGCTAGGGG | 4605 |
| hs132679256 | TTGGGTAACGAGATCACTAAGGG | 4606 |
| hs132677661 | TATATAGCTTCCAAGAATGAGGG | 4607 |
| hs132680607 | ACCCTTCACATGATAAAATGGGG | 4608 |
| hs132677303 | ATTACAAAAAGGACAAGCCATGG | 4609 |
| hs132681461 | ACCAAACAGTGGCATTGAAGAGG | 4610 |
| hs132677782 | CACAGGCATCTAGATATGGCAGG | 4611 |
| hs132683113 | TATATGTTGCGGCAATGAAGAGG | 4612 |
| hs132681341 | GTCTTGATGATGCCAATGCTAGG | 4613 |
| hs132682127 | TACCAAGTTTCCATTTACAAGGG | 4614 |
| hs132678319 | TAATTTGTCTTGGAATGATGCGG | 4615 |
| hs132682146 | CAACATAATAGGACAGCCCAGGG | 4616 |
| hs132680330 | GTATTACCGGTTGTCTAAGGGGG | 4617 |
| hs132679403 | GGATTGATCCCAGGTACATGTGG | 4618 |
| hs132678674 | GTGTAATGTTATGAAAGAAAGGG | 4619 |
| hs132678734 | ACAAACAGATGAGTTTCAGTGGG | 4620 |
| hs132679462 | GCAGATCAGAGGCTTCAGTAGGG | 4621 |
| hs132677985 | CTATGCCTCTCACAGCCAGAAGG | 4622 |
| hs132681182 | AGTTTGTAAGTATATTAGGAAGG | 4623 |
| hs132680423 | CATTAACTCAATCAGAGTCCCGG | 4624 |
| hs132683247 | TAAACCCAAGGCTTTAAGAGTGG | 4625 |
| hs132678486 | AGACAAAACCCCAGAGAAAGGGG | 4626 |
| hs132682288 | ATAAAAGAGTGCCCAGATGAGGG | 4627 |
| hs132681153 | ATATTTTGTACACATATGAAAGG | 4628 |
| hs132681875 | ACATAAAGAAGACTTAAACTAGG | 4629 |
| hs132679538 | TGCCATGGCATGAAATCCAAAGG | 4630 |
| hs132677705 | TCAATGGTAGTTGGTCATGATGG | 4631 |
| hs132677055 | GTACCCACCCAGGTAGATTGTGG | 4632 |
| hs132680245 | GCTCAGAGCTCACCTACTAAGGG | 4633 |
| hs132679512 | AGTCTGACTTTGCTCAGTGAAGG | 4634 |
| hs132681362 | TTCTCTTGTAGATCACCAGTGGG | 4635 |
| hs132677418 | TCTATGAGTACTCAGTTCCAGGG | 4636 |
| hs132681971 | GCTTACTAATGGATGTCACTAGG | 4637 |
| hs132682231 | GTCCTGCTAGATCAGGCATAGGG | 4638 |
| hs132678704 | CAGCTGATCTATCACGTCATGGG | 4639 |
| hs132680853 | GCTTAGGGAGATCACTCTGTTGG | 4640 |
| hs132677938 | TGACTACACTTCTGACACCCTGG | 4641 |
| hs132682000 | ATTGTGGAGAGAACTACGCTAGG | 4642 |
| hs132678488 | ACAAAACCCCAGAGAAAGGGGGG | 4643 |
| hs132682290 | GGGAGGATACACCCTCATCTGGG | 4644 |
| hs132681707 | CTCCTGGTAGCAAACAATGAGGG | 4645 |
| hs132681911 | AATTCCATTGGAATGACGGAAGG | 4646 |
| hs132683088 | ATCATGTTCTCAAGAAGACCAGG | 4647 |
| hs132678802 | TATGAAAGTGACAAAGCAAGTGG | 4648 |
| hs132678131 | GTTCCTAGCTGTACTTACTCTGG | 4649 |
| hs132679594 | AGATGTTTATATTCTGACGAAGG | 4650 |
| hs132679429 | AGTGTCAATAAAGGATTCAGAGG | 4651 |
| hs132677654 | CTTGATCTTAAGTATGGGGAGGG | 4652 |
| hs132680101 | CTGTATTCTGTAATTGCTAGGGG | 4653 |
| hs132678862 | TAGTAATGCTTATCTTGCCGAGG | 4654 |
| hs132677219 | ACATCAGACCGTGGGAACGGGGG | 4655 |
| hs132679759 | ATGTAATAGTCAATACGCCCAGG | 4656 |
| hs132680315 | GGAGGCAAGCATAGATCTTGGGG | 4657 |
| hs132681115 | CTGATGGCATAGGCTCCAGATGG | 4658 |
| hs132682003 | TTTGAAACAACAAGCAAGGAGGG | 4659 |
| hs132679114 | TTGTATGGTGTCTATATACGTGG | 4660 |
| hs132679661 | AATGTTATTGAGTTTATCTGTGG | 4661 |
| hs132679194 | TGTTAAACCTAGCAAAAGAGTGG | 4662 |
| hs132680766 | GCTTCAGCCTACCTTAGACTAGG | 4663 |
| hs132679420 | TTAAAATATTGAAATAGCGAGGG | 4664 |
| hs132681870 | GTGTATTTGCAGACTATCAGTGG | 4665 |
| hs132678314 | ATTAGCACATCTTAACCCATAGG | 4666 |
| hs132682673 | AATTGATCGCGATAGCAGTAGGG | 4667 |
| hs132679829 | GAGCTAAAAGGCTCCCCCAAAGG | 4668 |
| hs132677092 | TATGCTAGTACATCAGACATAGG | 4669 |
| hs132682968 | GCAAAGTACCTAAGTAACAAAGG | 4670 |
| hs132680461 | ACATTGGATGAGTAGACGAATGG | 4671 |
| hs132677878 | GGTTGGAGTACTTACAAGAACGG | 4672 |
| hs132679290 | ATGATGGCTGATATTTCCCATGG | 4673 |
| hs132683161 | AATGTGAGCCCATCAAAGGCAGG | 4674 |
| hs132678959 | AGTGGAGAAAAGTTCAGAGGGGG | 4675 |
| hs132680617 | AGAAGCATACAAAGTATACAAGG | 4676 |
| hs132681628 | GATTTGGCTTACAGGTTCAGGGG | 4677 |
| hs132681575 | TCCATAGTCAATGATGGCGATGG | 4678 |
| hs132677614 | AGCTTACGGCAAGGATGCAGGGG | 4679 |
| hs132679951 | TCTTCCGGAATACAATGTTGTGG | 4680 |
| hs132681208 | TAACATATACAATTTTACAAGGG | 4681 |
| hs132682576 | GAACAGAGATGTCATACAGATGG | 4682 |
| hs132683104 | AGCATGATAAGCATGATGTAGGG | 4683 |
| hs132679237 | ATAACTGCTTTGCCAATGTGAGG | 4684 |
| hs132681454 | TCTGTTCTCTGTTATAAAGGAGG | 4685 |
| hs132678961 | AAAGTGGAGAAAAGTTCAGAGGG | 4686 |
| hs132677467 | ATCATCACATTCAGGGGCAAGGG | 4687 |
| hs132678685 | ACAGAATGTGCCAAGCGTGTAGG | 4688 |
| hs132680506 | ATAAGGACTCTGAAATGCAAGGG | 4689 |
| hs132678273 | AATCAGGTAGGCAGGAAATGAGG | 4690 |
| hs132682252 | CATCAATCAGAAGTGGTGTAGGG | 4691 |
| hs132679614 | ATCACATGTCCAATATTACTGGG | 4692 |
| hs132681095 | CTCAAGAAGCAAACAAACCAAGG | 4693 |
| hs132679770 | TCTGAACTTCAAATACACTCTGG | 4694 |
| hs132680866 | CATCACACAATATATGGCCGTGG | 4695 |
| hs132678205 | AAAGAACCAAAAGGAACCCAAGG | 4696 |
| hs132678563 | CAGTTGAGACTACACCTTGGAGG | 4697 |
| hs132679218 | CAGAAGTGTCAAAGTTGAGAGGG | 4698 |
| hs132678859 | TAAGAGAAAATACAGTCTAGAGG | 4699 |
| hs132681837 | ACTACACAGAGAAAAGTTGTGGG | 4700 |
| hs132682437 | GTAGTAAGTACTGCTTTACATGG | 4701 |
| hs132678123 | CTGGCACTGTAGTTAAGAGATGG | 4702 |
| hs132677337 | GTTGGCCTCAAAGTAACAGAAGG | 4703 |
| hs132682415 | ACACTGCACTCAGTTGATAACGG | 4704 |
| hs132681964 | AAGGATACTTGGGAGCAGGTTGG | 4705 |
| hs132681354 | CACACCTCAGGAATGCCCACTGG | 4706 |
| hs132681267 | TAATGTATCCAAGACAACATTGG | 4707 |
| hs132682422 | ACTCCACTCCCTCTCTTGAAGGG | 4708 |
| hs132678722 | AAATTTGCTGTGATAAACAGTGG | 4709 |
| hs132678541 | GTAGATATTAAGGCCTGTGGGGG | 4710 |
| hs132678984 | TGTGGGTGGCAGTAAGGGAAGGG | 4711 |
| hs132680970 | AAATACACTTACTATCCTAAGGG | 4712 |
| hs132682976 | GAGTTCACATGCCAGTAAAGTGG | 4713 |
| hs132679634 | GTTAGAGAGAAATTATAGAGCGG | 4714 |
| hs132678004 | CCTGTGGTGATAAACAGCATTGG | 4715 |
| hs132683076 | GGAAAGGGATGAAGTTGTGAAGG | 4716 |
| hs132678253 | GTTCCCACTAGGAAATAGTAGGG | 4717 |
| hs132681682 | TTTAGAAATGGGATATCCTGGGG | 4718 |
| hs132680751 | ACCTGGACATTATGAAAGAGAGG | 4719 |
| hs132681200 | TTATTTCCTGGAACAGAACACGG | 4720 |
| hs132681813 | AAATAAGGAACTTTGTCCCAAGG | 4721 |
| hs132682398 | CATTTTGGAGATTCCATGTGTGG | 4722 |
| hs132681083 | TTTTATTTAATACTTTTGGGTGG | 4723 |
| hs132679652 | TACTGGGAGTCTCCGATAGTGGG | 4724 |
| hs132682007 | CAAGTAATATGGACAGCACAAGG | 4725 |
| hs132681642 | TAAACCTTGATTAAAAGCCAAGG | 4726 |
| hs132677508 | TTTTCCTTATGTGTCAGACGAGG | 4727 |
| hs132682568 | AACAACAAATATAAAAAAGGTGG | 4728 |
| hs132679492 | GCTACTGAATGTTTCATACCTGG | 4729 |
| hs132680283 | ATCTATCTCTTTAGGACCAGGGG | 4730 |
| hs132679478 | TTAACTATAATAGAGCCCTTCGG | 4731 |
| hs132677520 | CTTTGGGTGGTTAATCAAGTCGG | 4732 |
| hs132677836 | GTATCAAGGTCTCGATCGAGAGG | 4733 |
| hs132680502 | GGGGAAATAATACTTATCAAAGG | 4734 |
| hs132680785 | CTTGCCAGTTTTTTTCAGTGGGG | 4735 |
| hs132680685 | ATAACATTTCAAAATGCCCTGGG | 4736 |
| hs132677619 | GGTGGGTCTATCCAAGGCTGAGG | 4737 |
| hs132679122 | GGGAAGACTGGTATGTGTAAGGG | 4738 |
| hs132677997 | TTTATTAATGCCAGTGACTGCGG | 4739 |
| hs132679516 | TCTCTGTGTCTCTAACCTGAGGG | 4740 |
| hs132681725 | CATGTTTACAGGCATGGGAGAGG | 4741 |
| hs132677118 | TGTCTTTTGCCTGGAAACCAGGG | 4742 |
| hs132679136 | GAATTACAGCAGAGGAGGCGAGG | 4743 |
| hs132677510 | TTCCTTATGTGTCAGACGAGGGG | 4744 |
| hs132682010 | GGTACTAGAGTTAAAAGAAGAGG | 4745 |
| hs132677385 | GATTTCCATTAACGATGGTGTGG | 4746 |
| hs132681983 | AATTCCTCCTCCAAAAATCAGGG | 4747 |
| hs132677786 | ATCTAGATATGGCAGGCAGTAGG | 4748 |
| hs132677610 | TACAGTCTAAAATCAAGAGAAGG | 4749 |
| hs132679490 | GTTTCATACCTGGTATCATGTGG | 4750 |
| hs132679456 | GTTTCTTGCACTAAAAGATGAGG | 4751 |
| hs132678463 | TGGAGGATGAAGAATGACAAGGG | 4752 |
| hs132682434 | TGATTTGATCACAGTTCAGGTGG | 4753 |
| hs132679476 | GAAACAGTTTTCCTGCCGAAGGG | 4754 |
| hs132681798 | TAGGCAAGCTGCTACAAGGAAGG | 4755 |
| hs132677162 | TTTTGCTCCTGGCCAAAGGAGGG | 4756 |
| hs132682638 | AAAAAATGTTCAATCAAAGGTGG | 4757 |
| hs132682878 | ATATAATTCTAGGAATCCATAGG | 4758 |
| hs132682753 | TGAATAATAATGGGAAATGGAGG | 4759 |
| hs132682485 | TCTTGCTTCACTTTAATGTGAGG | 4760 |
| hs132682676 | ATGTACTTACATGATTATGTAGG | 4761 |
| hs132678736 | TCCCCTACTTTTCCTACTGTAGG | 4762 |
| hs132681458 | TTACATCCATGAAGCAGCAAGGG | 4763 |
| hs132680693 | AATGAGGATTCTCAGTTAAGGGG | 4764 |
| hs132678112 | CCTACCACGGGGGTATGCATGGG | 4765 |
| hs132678883 | CCTAGGGTATAATAGGAGGCAGG | 4766 |
| hs132678718 | CAAAGAGTAAATTACTGAGGAGG | 4767 |
| hs132678920 | TCTCCAGTGATACTTTAACATGG | 4768 |
| hs132681762 | CTCAGGACAAGTAGATAGGGAGG | 4769 |
| hs132678501 | TGCAGGATCTACCGGAAAGTAGG | 4770 |
| hs132677839 | AGTTCAAGAGTCGATGAAGAGGG | 4771 |
| hs132679623 | CATAACATGTTATAAAACATAGG | 4772 |
| hs132680355 | ACCTCCTATGTCCACCCCCACGG | 4773 |
| hs132681446 | TACATAGATCTTGTATCCCAGGG | 4774 |
| hs132679991 | GATACAAATAATGTAGCGAAGGG | 4775 |
| hs132677445 | TAGACAGACCCTTACAGGCATGG | 4776 |
| hs132677086 | AGATAGGTGATGAAAAAGCTGGG | 4777 |
| hs132681922 | AGATGTAGATGGGATCTCACTGG | 4778 |
| hs132681855 | GCAAGGCAGCTATATAGCAAAGG | 4779 |
| hs132678281 | CTATTACCATGAAATACACTAGG | 4780 |
| hs132679273 | CAAATTCTATGAGGTAGAGAGGG | 4781 |
| hs132678884 | CCCTAGGGGTGTAATCAAGAGGG | 4782 |
| hs132680151 | TAGTTTCCGTACTCCCCTAGTGG | 4783 |
| hs132683039 | TTTAGAATTCACTTACACAATGG | 4784 |
| hs132677741 | AGAGAAGGAGATTTACCTGATGG | 4785 |
| hs132677327 | ATACATTAGATTTATGCAGAGGG | 4786 |
| hs132677128 | GACAAGACCTGTAAATGCCCGGG | 4787 |
| hs132679794 | ACCCCCAGTAAGAGAGAAGGAGG | 4788 |
| hs132681493 | TTATGAACCTGTGTAATCATGGG | 4789 |
| hs132680732 | TCTCACTTCTTATTAACAGAGGG | 4790 |
| hs132683168 | GAACTGAGTGGGCTCAGTGTGGG | 4791 |
| hs132682121 | ATAGATACAAGTATATTGAAAGG | 4792 |
| hs132680505 | GAGTCCTTATCAGTATTCAAAGG | 4793 |
| hs132681731 | TTAGAATAATCAGGAACCAGTGG | 4794 |
| hs132680728 | TTCTTTCCCAAGGCCAAGAGGGG | 4795 |
| hs132682775 | AGCAAGAAGGTAACCAAAAGGGG | 4796 |
| hs132681321 | CAGGCAGAAGGCATTTCCAGAGG | 4797 |
| hs132679959 | GATCTTGTTACCTGAATCAGGGG | 4798 |
| hs132677823 | GCATCACTCAATACCATGGTAGG | 4799 |
| hs132677306 | AATTACAAATCCCTAGACATTGG | 4800 |
| hs132679457 | TTTCTTGCACTAAAAGATGAGGG | 4801 |
| hs132677577 | GGTGACAGTTTTAGTTCACAGGG | 4802 |
| hs132680910 | ATTCCATCAGAAAAAATGGTGGG | 4803 |
| hs132682268 | ATGTATACTTAGGTGTCTGTAGG | 4804 |
| hs132683237 | TACAAAATCAGACCCATAGAGGG | 4805 |
| hs132679065 | TCTGAACATGTGTAAAGCAAGGG | 4806 |
| hs132682321 | ACTTATTATTTTGTCATACACGG | 4807 |
| hs132679216 | TTTACTATATATATAAGTTGGGG | 4808 |
| hs132682841 | CTAGCGAGCTAAACCAAAGAGGG | 4809 |
| hs132677142 | GTGGCTGGATTCACAAAGCTGGG | 4810 |
| hs132679502 | CGAGATTCTTTCCTCTCAGGGGG | 4811 |
| hs132678771 | TCCAGAGGCACACACTGGAATGG | 4812 |
| hs132680762 | CCATATGCCTAGTCTAAGGTAGG | 4813 |
| hs132682012 | TTCAATCTTTTCCAAAGCCGAGG | 4814 |
| hs132677545 | ATACACACACTTTTTTTTGGGGG | 4815 |
| hs132678142 | TAAAAAATATCTTCTATCAGAGG | 4816 |
| hs132678935 | ATGTTTTTAGAAAACATCACAGG | 4817 |
| hs132677911 | GACAATGCTAGTTTTAGTAGGGG | 4818 |
| hs132682320 | TTCCATTGAAGACATTAAGACGG | 4819 |
| hs132680422 | TACATAGTCACTTTATAAGAGGG | 4820 |
| hs132683050 | TACCTTCTTGGCATCAAACAGGG | 4821 |
| hs132681465 | GGTTGACTGACAGTGGTCTAGGG | 4822 |
| hs132680897 | CTGAGAAAATCATCAGTCCAGGG | 4823 |
| hs132683248 | AAATATGGCCCTTAAACCCAAGG | 4824 |
| hs132680689 | AACAATGAAACATTTTACCCAGG | 4825 |
| hs132680942 | TGTTCTGCAGCAGGTTAAGTGGG | 4826 |
| hs132678990 | CTTTTATTTACTCTTGTGGGTGG | 4827 |
| hs132681379 | GAAAAGGGAGAGTGAGAACGAGG | 4828 |
| hs132679601 | TTTACTTTACCTCTGACAGTAGG | 4829 |
| hs132679771 | CATTTGTCTCAGAGCAATGATGG | 4830 |
| hs132681202 | TTCAGTTTGTGAATGTCATGTGG | 4831 |
| hs132681293 | CAACCTTCCCATATGTGTGGAGG | 4832 |
| hs132678662 | TTTGTCTAGTGACTGACAGAAGG | 4833 |
| hs132680060 | GATTTGAAGAAGAATATGGAGGG | 4834 |
| hs132679183 | GGTTTAATTTTGGATAAGTGGGG | 4835 |
| hs132681770 | ATCTGAGAGGACGACAGCTAGGG | 4836 |
| hs132679461 | AGCAGATCAGAGGCTTCAGTAGG | 4837 |
| hs132677737 | ATTTAAACTAATCTTAGTGGTGG | 4838 |
| hs132681174 | GGCATACAGGAATTTTTTGGGGG | 4839 |
| hs132679206 | GTAGGAAGTACATAAGACATAGG | 4840 |
| hs132681135 | CCACCAATTTAACTGTAACTCGG | 4841 |
| hs132677511 | TCCTTATGTGTCAGACGAGGGGG | 4842 |
| hs132682692 | ACATTATGTAAGACTAGCCCAGG | 4843 |
| hs132680514 | ACCCACCTATTCATTTCCCAAGG | 4844 |
| hs132679861 | AAGTACAGTTCTCTTTCTAACGG | 4845 |
| hs132680114 | TTTAAAGTTAATTTTATAGGGGG | 4846 |
| hs132679646 | TTGTCTTTCTGGAAATGAGGAGG | 4847 |
| hs132683106 | TCACATGATATTGCCCAAAGAGG | 4848 |
| hs132682919 | GCATCGTTTTGGAGTGTGCATGG | 4849 |
| hs132682994 | AGTCCTATGAGAAGCAGGCAGGG | 4850 |
| hs132681946 | GAAAAGTGAAGACATTTGAAGGG | 4851 |
| hs132679570 | TTACTTTATGTAGCTACTGAGGG | 4852 |
| hs132680589 | GTAGCAGAGGTAAATTGCCATGG | 4853 |
| hs132678882 | CACCCCTAGGGTATAATAGGAGG | 4854 |
| hs132682232 | AGTCCTGCTAGATCAGGCATAGG | 4855 |
| hs132679781 | TAGTGATAAATCGAATAAGTTGG | 4856 |
| hs132681124 | TTCCTAATTCTTGATCCAAGTGG | 4857 |
| hs132677407 | GCATTTGAGATAGCCAGTGATGG | 4858 |
| hs132679031 | CCTTTAAATGAAGATCACATTGG | 4859 |
| hs132683162 | CTAAAATGTGAGCCCATCAAAGG | 4860 |
| hs132680456 | TGAGTTGGTAAACAACCTTAAGG | 4861 |
| hs132680459 | TGGAGCCTAGACAGAGACATTGG | 4862 |
| hs132677290 | TAAAGTGAACCAAATCAAAGTGG | 4863 |
| hs132678846 | GCTAAGGCCTGGAAGCCTCAAGG | 4864 |
| hs132682770 | GCTCTTGTGGTTACAACAAGAGG | 4865 |
| hs132681987 | TCTACCCTGATTTTTGGAGGAGG | 4866 |
| hs132679459 | AAGAAACCCAGCAAGCTGAAAGG | 4867 |
| hs132681335 | AATATAACCTCAATTGCAAGAGG | 4868 |
| hs132681842 | TGCCAGTTCCCTGTGCTAAGAGG | 4869 |
| hs132680458 | AGGTTGTTTACCAACTCAGTAGG | 4870 |
| hs132677509 | TTTCCTTATGTGTCAGACGAGGG | 4871 |
| hs132678996 | GGGATTCTGAAGAAGTTCCTGGG | 4872 |
| hs132678184 | CCTAAACTTTGGTGTGCAGGAGG | 4873 |
| hs132677712 | CCATGGGGTAGGATGGTTCGAGG | 4874 |
| hs132679571 | CTTACTTTATGTAGCTACTGAGG | 4875 |
| hs132679276 | AGTTAATATCAAATTCTATGAGG | 4876 |
| hs132681076 | TACTTTTAGTGTCAACAAAGAGG | 4877 |
| hs132682582 | TTTCATGGAACAAAAAGTCACGG | 4878 |
| hs132679830 | CCAAAGGACTGAAACTTGAAAGG | 4879 |
| hs132682334 | TTAGGATTAGCTTATCCCAAAGG | 4880 |
| hs132682855 | AAAAATGTCTTCGATCTACAAGG | 4881 |
| hs132681491 | TGTGTAATCATGGGTAGTGTTGG | 4882 |
| hs132682659 | CAGCTAGTAATGTAAAATGGTGG | 4883 |
| hs132679284 | ACATTAATATCATCATCAAACGG | 4884 |
| hs132681276 | GAAGCACGAATATAAAACAAAGG | 4885 |
| hs132680872 | TAGGAATAAGTAATTCTGATGGG | 4886 |
| hs132682849 | CTGACTGACATAATAACTGCAGG | 4887 |
| hs132677673 | CTATCTCGTAAATAAGAGCTGGG | 4888 |
| hs132679208 | ATCATAGATTCTTTGCTAAGGGG | 4889 |
| hs132681796 | ATTATACACAGAAAAGCTGCAGG | 4890 |
| hs132677480 | ATTGTTATATCATTTGACATCGG | 4891 |
| hs132681556 | GTGTGTACATATATGGGTGGTGG | 4892 |
| hs132678103 | GCTTGGCTAGGGTTGTATTGGGG | 4893 |
| hs132677498 | AAGGAACTGGCACTTTTCAGTGG | 4894 |
| hs132681184 | TTCTGATGACCCTAGATCCAGGG | 4895 |
| hs132677386 | AACTTGATTTCCATTAACGATGG | 4896 |
| hs132678759 | CAGGAGCAGAGCCTTGAGATGGG | 4897 |
| hs132678937 | TTTATTTTCAGATAAAGCACAGG | 4898 |
| hs132682103 | AAAAAGCTAGTAAATGGAAGGGG | 4899 |
| hs132677819 | GATCCTTGCACAAACAAGCCAGG | 4900 |
| hs132682367 | TGGTTTTGTCTGGGAATCGGTGG | 4901 |
| hs132677236 | CTATAGAGTAAGTTGCAGGCAGG | 4902 |
| hs132681920 | AAATACTGAAAGATGTAGATGGG | 4903 |
| hs132677296 | CTTTTAACCTGAAAATCCAAAGG | 4904 |
| hs132680680 | ATTCAGTGTCAGGACAAAGATGG | 4905 |
| hs132680323 | AGTGTCTCCAGTGCCAGAGAGGG | 4906 |
| hs132679277 | TTGGTCTTGCAAAGGGTCAAGGG | 4907 |
| hs132681949 | CTAGGCTGAAGGATGATAGTGGG | 4908 |
| hs132677438 | GATGACTTCCCAGTAGGTGGAGG | 4909 |
| hs132681704 | ATAAAACTTGTTGAATCACTCGG | 4910 |
| hs132679905 | ATATTTTCTCACTCTTTCAGAGG | 4911 |
| hs132678020 | CAAAGTGGTAATTTGCCAGTAGG | 4912 |
| hs132679138 | AGAGGAGGCGAGGTAGAGGAAGG | 4913 |
| hs132681503 | CACTGCCTTTCAGTACCTACGGG | 4914 |
| hs132682311 | CCATTTTAGTGATTTCTGGAAGG | 4915 |
| hs132681035 | ATTGCTAGTCCCCACTTACAAGG | 4916 |
| hs132680441 | GGAAATTCAAGAAGGGATGACGG | 4917 |
| hs132679979 | ACAACTATGAAAAAGAGCATTGG | 4918 |
| hs132682668 | AGTGTCCTTACACTATTCGAAGG | 4919 |
| hs132677975 | ATGAAAAGAGTGGTGAGGTAGGG | 4920 |
| hs132679546 | TGTTTACTATCCCAAAGTTGGGG | 4921 |
| hs132677357 | CCATATCAGAAAATAAATGAAGG | 4922 |
| hs132683225 | AATAAATAAAAAGTAAACCTAGG | 4923 |
| hs132682671 | GTTAAAATCAGTCACTTCAGAGG | 4924 |
| hs132678505 | CTGCATGTAGATCCTTATGTGGG | 4925 |
| hs132681028 | TTCATCACTATCATAGAGATGGG | 4926 |
| hs132682098 | TGCTTGTGTAATTAACTCCATGG | 4927 |
| hs132677805 | CATTATTCAGATCATTGCAAAGG | 4928 |
| hs132680752 | TGTCCAGGTGAAAGAAATGCCGG | 4929 |
| hs132682418 | AATCTCTTTCCCTTCAAGAGAGG | 4930 |
| hs132682620 | CCAATTATAGCTATCTAGCTCGG | 4931 |
| hs132678197 | TGCTTGTGATCAAGAATGGATGG | 4932 |
| hs132677987 | TTCTGGCTGTGAGAGGCATAGGG | 4933 |
| hs132681764 | GATTTTGTTTAGAAGTAGCAAGG | 4934 |
| hs132679667 | TTTGGTAGAGATAGGCAGTAGGG | 4935 |
| hs132681347 | GAAAATGTGTTACAGTAAGGAGG | 4936 |
| hs132680059 | TGATTTGAAGAAGAATATGGAGG | 4937 |
| hs132679378 | TGCAAAAGTAAAGAAAAAGTGGG | 4938 |
| hs132679421 | CTTAAAATATTGAAATAGCGAGG | 4939 |
| hs132679677 | GCTAAAATATATGTGACTGTAGG | 4940 |
| hs132679742 | GTTTCCTTAAAGCCCTCTGAGGG | 4941 |
| hs132677713 | GTGTGATCCATGGGGTAGGATGG | 4942 |
| hs132681622 | AGAGGTTTGAGATTCACAAAAGG | 4943 |
| hs132678348 | CGTAATTGTTTCCCTTGAGGGGG | 4944 |
| hs132680439 | CAGTGTTAGGAAATTCAAGAAGG | 4945 |
| hs132679417 | TCTTCATCAACAGTTTGGAAAGG | 4946 |
| hs132677693 | TGTCACCACCAACGATGACTGGG | 4947 |
| hs132678263 | TACCACCCAGGGAATACAGCAGG | 4948 |
| hs132680127 | TTGTTACTAGAACTAAAATGAGG | 4949 |
| hs132680063 | TCAAATCAAAAAACGTAGAACGG | 4950 |
| hs132678311 | ATAAAGACAATATTTCCTATGGG | 4951 |
| hs132679356 | CAATCTAGATAGTATTTCCGTGG | 4952 |
| hs132683233 | TTTGAGATTGGATCTCCTAGTGG | 4953 |
| hs132678700 | CATGTATCATTAGGGAGTAGAGG | 4954 |
| hs132682200 | AAACAATCTAAAACTTGCAATGG | 4955 |
| hs132677250 | CTAAAACTGGTGAATGCAGATGG | 4956 |
| hs132679695 | GGACTTCTAACTTAAGACAATGG | 4957 |
| hs132680921 | TTTTAGGAACTTCTGGGCTGGGG | 4958 |
| hs132679899 | GTAATCCACTGAAGGAACTCAGG | 4959 |
| hs132677100 | ATTACATAGAAGCATTAGAAAGG | 4960 |
| hs132680507 | GATAAGGACTCTGAAATGCAAGG | 4961 |
| hs132677646 | TCCCCATACTTAAGATCAAGTGG | 4962 |
| hs132681781 | AGATGTTTATTTTAGGACTGTGG | 4963 |
| hs132680347 | CATTAAATGTAAATAATCCGTGG | 4964 |
| hs132679733 | CACCAAATACAGGCAGTCACAGG | 4965 |
| hs132680124 | TATAAAGGAAATGGGTTCAGTGG | 4966 |
| hs132681786 | TCTAAAGAAGATGGATCACCAGG | 4967 |
| hs132679537 | TTGGATTTCATGCCATGGCAAGG | 4968 |
| hs132679591 | GTTCTATTTTGGAGAGAAGGAGG | 4969 |
| hs132677350 | TATGGCCATATAAATTTCAAAGG | 4970 |
| hs132677830 | CTAAGCTCCCTCTGGAATGGAGG | 4971 |
| hs132681840 | AAGAACTCTTGGGCCAGTGTAGG | 4972 |
| hs132681074 | ATAGGTTTGGTGGACAAACTAGG | 4973 |
| hs132681471 | TAGACAGAGGTTTCCCTCCACGG | 4974 |
| hs132681574 | TGTTACTCCATAGTCAATGATGG | 4975 |
| hs132681861 | GCATGAGCTTCATACTCACTTGG | 4976 |
| hs132678517 | GTTTCTGAACTAGCAGTGCCCGG | 4977 |
| hs132680215 | GGAGATGGTTTTGAGTACTCAGG | 4978 |
| hs132681313 | TCTTACACTAGTACCCTTCAGGG | 4979 |
| hs132681410 | TGCTACAGAAGTATAAATGATGG | 4980 |
| hs132681916 | AATACCTTCCGTCATTCCAATGG | 4981 |
| hs132682711 | CCTACTGTTAAGACACAGGATGG | 4982 |
| hs132680525 | AATTCTACAGTAACTGGGGTTGG | 4983 |
| hs132679729 | ATGATTAGAATAGGGAACCTAGG | 4984 |
| hs132678757 | CTTAACACCCCGTTGACCAAAGG | 4985 |
| hs132682001 | GATATTTGAAACAACAAGCAAGG | 4986 |
| hs132681495 | ATTGTAACCTAAACTGCCTGTGG | 4987 |
| hs132677163 | ATTTTGCTCCTGGCCAAAGGAGG | 4988 |
| hs132678470 | GGCCCTTTCATATCTCTGTACGG | 4989 |
| hs132682891 | ATTGCAGTGAAAGAAATTTGCGG | 4990 |
| hs132679321 | AGGTGTGATCTAACCAGCATAGG | 4991 |
| hs132681450 | TTATTACAAGTCACTCTTTGGGG | 4992 |
| hs132679679 | TGACTGTAGGAATCCTCTGTTGG | 4993 |
| hs132678879 | TCCCTCTTGATTACACCCCTAGG | 4994 |
| hs132679360 | GTATTTCCGTGGGGGTAATGAGG | 4995 |
| hs132680917 | GCTGGGGAAATGTGGCCTGAAGG | 4996 |
| hs132678151 | TTCTTACAGCTCCTTCACATTGG | 4997 |
| hs132680997 | GCTTACTTTAGAGCAGTCACAGG | 4998 |
| hs132678059 | CTATATGCAGAATCGTGTATGGG | 4999 |
| hs132679480 | TTTGATGTTATGTACTTAGGAGG | 5000 |
| hs132683259 | ATTAGGAATGCAATATATGGAGG | 5001 |
| hs132681001 | TGTCCCTGATGCTGGAAAAGTGG | 5002 |
| hs132679053 | AATTTGTGTTTCAAAAAACTGGG | 5003 |
| hs132680576 | GAATAGGATGGTTTGTAAAGGGG | 5004 |
| hs132679155 | TGAAGATCTAAAGTGAAGCCAGG | 5005 |
| hs132677755 | CTGTAGGTCCTCAGGACACATGG | 5006 |
| hs132677708 | GCATGAACTGGCCATGAACATGG | 5007 |
| hs132677601 | AAGAGGTAGGTCATTCAGCCAGG | 5008 |
| hs132682853 | TCTTCGATCTACAAGGTCTACGG | 5009 |
| hs132681246 | TCTTATGCCTAGATGTACATAGG | 5010 |
| hs132678536 | GGAAAGGCCAGTCTATTGACAGG | 5011 |
| hs132681052 | TTATTGTTTGCTCCATACAAAGG | 5012 |
| hs132680918 | CAGAAGTTCCTAAAATCTTGTGG | 5013 |
| hs132681077 | ACTTTTAGTGTCAACAAAGAGGG | 5014 |
| hs132683094 | TTATATGTTTTGTTACAAGTAGG | 5015 |
| hs132679715 | GTGAAAAGTAATACTTCCTAGGG | 5016 |
| hs132679141 | GGGCATTTTGTGCAATGCTGAGG | 5017 |
| hs132681683 | ATAAACGTATTTTTCCCTTGAGG | 5018 |
| hs132682284 | CATGTAGTCTCTAAAGGCACAGG | 5019 |
| hs132677456 | TGCGAGTTACACACATCGACTGG | 5020 |
| hs132677502 | CAACTACACCGAATCTCACATGG | 5021 |
| hs132680991 | ACACCCCATCAACAATAAAGTGG | 5022 |
| hs132680847 | CAGTAGTTGTGGCAACCTATGGG | 5023 |
| hs132679772 | ATAAATGCAGTAAAAAGCCTAGG | 5024 |
| hs132678864 | GTGATCATTCATTTAAACCTCGG | 5025 |
| hs132678147 | CATCTTTGAAAAATTCAAGAGGG | 5026 |
| hs132681735 | TTCTTCCTGATTACATCCAGTGG | 5027 |
| hs132680443 | ATGGATTTGAAAGGACAAGAGGG | 5028 |
| hs132680932 | TCTAGACATATAATTTTAAGGGG | 5029 |
| hs132679188 | TTTCCATTTGAATATCCCACAGG | 5030 |
| hs132678896 | CTAAAAGACACACTACTGATGGG | 5031 |
| hs132677621 | ATTGCAGGTTGGAGGAGGGTGGG | 5032 |
| hs132682526 | TGCCTTTTCCAGGTTTTCTGAGG | 5033 |
| hs132677038 | AATTAATCTGCCGAAGATGACGG | 5034 |
| hs132678770 | GGCACACACTGGAATGGCACCGG | 5035 |
| hs132681025 | ACTGGAAGAGATATACAAGATGG | 5036 |
| hs132678137 | TTTAAAATATTCTGTAGCCAAGG | 5037 |
| hs132680928 | TTGCTCGGACAGAGTCTGTAGGG | 5038 |
| hs132678066 | GACTGGGGATACAATGTGGATGG | 5039 |
| hs132678692 | AAGTCTCCCACACATGATGCAGG | 5040 |
| hs132677408 | ATAGCCAGTGATGGAAATGAAGG | 5041 |
| hs132679160 | TAGTAAGGATGGATAAGGAGTGG | 5042 |
| hs132681330 | TATTAGACACAAAAATTGGAAGG | 5043 |
| hs132681302 | CCTCCAAGCATATCAAGTACAGG | 5044 |
| hs132682075 | GGAATTTTCTTTAGCTGGCAGGG | 5045 |
| hs132682838 | TATTTTCAATGATTTTCCATGGG | 5046 |
| hs132678638 | CAAACTATGACGCGAGAGGATGG | 5047 |
| hs132680455 | GTAGGTAGTATGTTATCCTCAGG | 5048 |
| hs132681322 | TGACGCTTCTTGCAGGCAGAAGG | 5049 |
| hs132682439 | GTTTACTGAAAGGCATACCTAGG | 5050 |
| hs132678880 | CCCTCTTGATTACACCCCTAGGG | 5051 |
| hs132679211 | CCAGTTACCATAAAAAGACATGG | 5052 |
| hs132679897 | TTTTACCTGAGTTCCTTCAGTGG | 5053 |
| hs132681147 | TAGAGATATAATAAGTGGAAGGG | 5054 |
| hs132678833 | TCAGACACTCCCATTCCAATGGG | 5055 |
| hs132678071 | TGAAAAGGATGCGGGAAAGGTGG | 5056 |
| hs132680676 | AATTAGAATTGTCTTTCAGAGGG | 5057 |
| hs132677825 | CTCAATACCATGGTAGGAGGAGG | 5058 |
| hs132678119 | CATCTCTTAACTACAGTGCCAGG | 5059 |
| hs132678422 | GCCACCACAACAGAGAAGGTTGG | 5060 |
| hs132683148 | TTATTTTGTAAAGCTACGGAAGG | 5061 |
| hs132681507 | GTTTCCTGAACCAGGAACATAGG | 5062 |
| hs132683210 | AACAATATATAAATAGAGGATGG | 5063 |
| hs132682280 | TTCTACCCTGCTACTTGTAGAGG | 5064 |
| hs132680735 | ACCTGATTTATTCAAAGAGGAGG | 5065 |
| hs132682196 | ATTAAAATAACCAATCCTCTAGG | 5066 |
| hs132680849 | GCAACCTATGGGATTAAGTAGGG | 5067 |
| hs132678878 | TCATAGCATACCAATTAAGCAGG | 5068 |
| hs132680004 | TTATAGGAAGAAAGTTATGAAGG | 5069 |
| hs132678832 | GTCAGACACTCCCATTCCAATGG | 5070 |
| hs132677115 | CAAAGTTTATATGTGATCCAAGG | 5071 |
| hs132680374 | ACAGCCCACTCCTAGCCCATGGG | 5072 |
| hs132683145 | CGTAGCTTTACAAAATAACAAGG | 5073 |
| hs132678465 | GACCGTACAGAGATATGAAAGGG | 5074 |
| hs132682246 | GGACTGCAAAGCACTCAAGCAGG | 5075 |
| hs132677615 | AAGCTTACGGCAAGGATGCAGGG | 5076 |
| hs132681101 | ATATTGTGATTTAAGGGCGTAGG | 5077 |
| hs132678741 | CCAATAAATCAACCTACAGTAGG | 5078 |
| hs132679629 | TTTTCAACTGGTAGTTGGGGAGG | 5079 |
| hs132678328 | AGCCTACTAAAGAGAATGAAAGG | 5080 |
| hs132682599 | TATTGCCACGGAAGAAGGCTGGG | 5081 |
| hs132681136 | CGAGTTACAGTTAAATTGGTGGG | 5082 |
| hs132678730 | CAATATTTAAGAAAAAACTTAGG | 5083 |
| hs132678278 | TAACAACAATTAGTTAAAAGCGG | 5084 |
| hs132677365 | TCACCAGAGATAGAAATATGAGG | 5085 |
| hs132683132 | TAACATATCAATAAATTACATGG | 5086 |
| hs132679094 | TATTAATCCATCCCTGGCATAGG | 5087 |
| hs132677035 | ATAGCTTTAGTTTTCAAAAGGGG | 5088 |
| hs132681275 | AAGCACGAATATAAAACAAAGGG | 5089 |
| hs132679682 | CACCAACTTATCTATTGCAAGGG | 5090 |
| hs132680284 | CATCTATCTCTTTAGGACCAGGG | 5091 |
| hs132679799 | TCCTCCTTCTCTCTTACTGGGGG | 5092 |
| hs132680348 | ATTAAATGTAAATAATCCGTGGG | 5093 |
| hs132678005 | GTGGTGATAAACAGCATTGGAGG | 5094 |
| hs132678275 | GCTGTTGAGTGACAGTTAAGTGG | 5095 |
| hs132678473 | CCAAGGAACAAGGTCCCTATGGG | 5096 |
| hs132677687 | GACTGGATCCCAGTCATCGTTGG | 5097 |
| hs132677734 | CATTTTAAAACTCTTAGTGGTGG | 5098 |
| hs132679768 | TGATAGATAAATGTAAAGCTGGG | 5099 |
| hs132678354 | GTCTAGATTTGGAGAGACAAGGG | 5100 |
| hs132680950 | AGTAGAAAAATTCTTCCCATGGG | 5101 |
| hs132681522 | AGGGTACTCTTTAAATCTAGTGG | 5102 |
| hs132679292 | GAAGACTACAAGCCATATGATGG | 5103 |
| hs132681372 | TATTTTTCATCTTTATGCGATGG | 5104 |
| hs132681655 | TGGTGAACTCTGGTCTTCCATGG | 5105 |
| hs132681383 | AGTGAGAACGAGGATGAGGGAGG | 5106 |
| hs132681474 | GTAGCTGATAGAGGTGAGAAAGG | 5107 |
| hs132679540 | AGATGGATTTTACCTTGCCATGG | 5108 |
| hs132681598 | ACTTGGTCACACTATATAAGTGG | 5109 |
| hs132677843 | AATCTGTTAAATAGAAACATAGG | 5110 |
| hs132678723 | AATTTGCTGTGATAAACAGTGGG | 5111 |
| hs132681719 | AAAGTAAACTTAAAATTAGGAGG | 5112 |
| hs132680850 | TGGACCCTACTTAATCCCATAGG | 5113 |
| hs132679699 | AGAGCAGTGTGTAAAAGGATTGG | 5114 |
| hs132678376 | TGTCCTTATAGCTTCTTGGTGGG | 5115 |
| hs132679152 | AAATCAGTCTAAATACTGCCAGG | 5116 |
| hs132679162 | GGATGGATAAGGAGTGGGAGTGG | 5117 |
| hs132680409 | GCCTAGTCCCAATTTCTGGTGGG | 5118 |
| hs132682840 | GTTTAGCTCGCTAGCTAACAGGG | 5119 |
| hs132681966 | TTGTACTATGAAGGATACTTGGG | 5120 |
| hs132677883 | TACAGAATAAGAAAGCACTAGGG | 5121 |
| hs132680543 | CCACAATTAAGAAAAGATGATGG | 5122 |
| hs132681872 | AGTTACAATGAGTTCTTCAAGGG | 5123 |
| hs132681504 | GCACTGCCTTTCAGTACCTACGG | 5124 |
| hs132682293 | TACTAGATATTACTCACTCAGGG | 5125 |
| hs132677762 | CTGCTTTGCTCAAATGTCAGGGG | 5126 |
| hs132681467 | AGTTTGAGCTGCAGCACCCGTGG | 5127 |
| hs132680758 | TAGCACAGATACAATTAAGCAGG | 5128 |
| hs132677364 | AGAGATAGAAATATGAGGCATGG | 5129 |
| hs132679550 | TCCCAAAGTTGGGGGGACATGGG | 5130 |
| hs132682239 | GCTGAAGCTGACGCTGACACGGG | 5131 |
| hs132680641 | AAAAAAATGTCAACGAAGCAGGG | 5132 |
| hs132679494 | TTTGACAACGTGTCTCAAGAAGG | 5133 |
| hs132677605 | TTTAAATTTAAGGTTCAAAGAGG | 5134 |
| hs132682721 | TCCCTGCCAGAACAAGCAAAAGG | 5135 |
| hs132677812 | TCTTGGCAGGAAACAATTGATGG | 5136 |
| hs132680399 | TTCCTTTATCCAGTTATCTGAGG | 5137 |
| hs132683058 | TCAGAACTAAAGTTTTTGAAGGG | 5138 |
| hs132679309 | TGACATCTGAATCTATGAAAGGG | 5139 |
| hs132678304 | ATGCTCTTGCCAGATTTGTAGGG | 5140 |
| hs132682113 | TTAAAAGGTACCTACCCATAGGG | 5141 |
| hs132681936 | TCTCATATAGAAGCTATTGTGGG | 5142 |
| hs132681962 | TGGGAGCAGGTTGGAGAAGAGGG | 5143 |
| hs132679990 | GTATCTCTACTTCGTCTACTGGG | 5144 |
| hs132679032 | TTAAATGAAGATCACATTGGAGG | 5145 |
| hs132682703 | TATCCTGTCACATTACACTTAGG | 5146 |
| hs132681549 | CCTGTCAGTATTGACCCCTTGGG | 5147 |
| hs132678202 | ATACAGATTAAAGAACCAAAAGG | 5148 |
| hs132677260 | GGTGTATGTTGAAGATGAAAGGG | 5149 |
| hs132678076 | ATATCCCAAAACCTCCATTACGG | 5150 |
| hs132680191 | ATTGATGTACAAGCCCGAAAAGG | 5151 |
| hs132677058 | TGACTGGTATATTCCAAGAATGG | 5152 |
| hs132680363 | AGGATATGCCCCTTTATGATGGG | 5153 |
| hs132681092 | GGCTTGGTTTGTGGTCAAGATGG | 5154 |
| hs132680933 | TGTCTAGAAAAGCTTTTGCTCGG | 5155 |
| hs132681148 | TTAGAGATATAATAAGTGGAAGG | 5156 |
| hs132678012 | GTTGTCTAACCTTTTGGCAAGGG | 5157 |
| hs132677517 | ATCAAGTCGGTACACTCGGATGG | 5158 |
| hs132680998 | ACTGCTCTAAAGTAAGCTAAAGG | 5159 |
| hs132678013 | GGTTGTCTAACCTTTTGGCAAGG | 5160 |
| hs132679344 | TTAGTACCATAGTTATACTCAGG | 5161 |
| hs132678513 | TCATCTTGAGACAAGAGCTCAGG | 5162 |
| hs132677294 | CATTTGTGAAGGTGTTTAAGTGG | 5163 |
| hs132678181 | TATTATGTGTATAGTATGGATGG | 5164 |
| hs132678641 | GCCACGTGACACAGAAATGAAGG | 5165 |
| hs132677657 | TCCCACTTGATCTTAAGTATGGG | 5166 |
| hs132677462 | AGCAGTTGCAAAGTAAAGAATGG | 5167 |
| hs132678044 | TGCCATGGCTGTTTGTTAGAGGG | 5168 |
| hs132679757 | ATCTAGTGTTTGATTGAGGACGG | 5169 |
| hs132678074 | GCAAAAAATTGAAAAGGATGCGG | 5170 |
| hs132679374 | ATTCAATCAGTAGATAAGCTTGG | 5171 |
| hs132678084 | GTAGTTTGACATGACCGTAATGG | 5172 |
| hs132682604 | CTTGGCCTATCTCAGGGTACAGG | 5173 |
| hs132681102 | TATTGTGATTTAAGGGCGTAGGG | 5174 |
| hs132677363 | GAGATAGAAATATGAGGCATGGG | 5175 |
| hs132677749 | AATTGCATAGCAATCAAAAAGGG | 5176 |
| hs132680790 | GAAAAGAACAGAATGTCCTTGGG | 5177 |
| hs132680262 | TTTAGCTCTGTGCTAATGAAAGG | 5178 |
| hs132680149 | TCAAATATGGTCCTGCACACAGG | 5179 |
| hs132677169 | CAGACTTCAACTTTCTACCCTGG | 5180 |
| hs132677193 | AGAAGACTTATAAAAGACCTTGG | 5181 |
| hs132681071 | CTAGGAACTGGAGAATATATGGG | 5182 |
| hs132679178 | TAAGAAGAGGTGTAGTAGAGAGG | 5183 |
| hs132681854 | CAAGGCAGCTATATAGCAAAGGG | 5184 |
| hs132682182 | TTCTAGATCAGCTTTAACAATGG | 5185 |
| hs132681291 | ACACACACCTCCACACATATGGG | 5186 |
| hs132678660 | TGAAACTGTAACAGTTGTAAGGG | 5187 |
| hs132681652 | AAAGTTTGTCTAGGATGCCATGG | 5188 |
| hs132677258 | CACCATGTCTTTGTTGCAGGTGG | 5189 |
| hs132680873 | ATAGGAATAAGTAATTCTGATGG | 5190 |
| hs132679477 | CTATAATAGAGCCCTTCGGCAGG | 5191 |
| hs132680356 | ATTAGACGAGCAAAAGGAAGAGG | 5192 |
| hs132679095 | GGATACTCCTATGCCAGGGATGG | 5193 |
| hs132683109 | TGAGTCCTGGATTTTTAATGTGG | 5194 |
| hs132679650 | TCTTTGTCAGTTCCCACTATCGG | 5195 |
| hs132679782 | GATAAATCGAATAAGTTGGTAGG | 5196 |
| hs132677490 | TCTAATGTATTTGAAAAATGTGG | 5197 |
| hs132679582 | CAGAATAGCCCAGTCAAAAGAGG | 5198 |
| hs132682653 | AAGTAATTAAAATCTGAATGTGG | 5199 |
| hs132682430 | CATGTTAAATTAAGGTTTAGGGG | 5200 |
| hs132677287 | GTTCAAAAAGAAACACAGATAGG | 5201 |
| hs132677788 | TCTAGAATCTAAATAAGGCTAGG | 5202 |
| hs132682952 | ATATGCAAGCTAAATATGCTTGG | 5203 |
| hs132678196 | AATATGCTTGTGATCAAGAATGG | 5204 |
| hs132678619 | CTGACAACTCCAAGGCTGGATGG | 5205 |
| hs132681785 | CGACTTGGTTCTAAAGAAGATGG | 5206 |
| hs132683074 | GCTTCCTCAAACACAGGGAAAGG | 5207 |
| hs132679220 | GTAGGCCAAGCCAAATCAATCGG | 5208 |
| hs132679968 | AGTTACAGTCCAGTTACTTGAGG | 5209 |
| hs132681364 | GGGTCAACCAAAGTGTATAAAGG | 5210 |
| hs132678206 | GGAACCCAAGGAAATCAGAAAGG | 5211 |
| hs132683112 | TAATGTGGACATATATGTTGCGG | 5212 |
| hs132682713 | CCATCCTGTGTCTTAACAGTAGG | 5213 |
| hs132680906 | CTCACAGTAGTTTTACCCCATGG | 5214 |
| hs132679935 | AACATTATTACCAAAACCATCGG | 5215 |
| hs132681819 | AGTTATAGCTGACCTTTACTGGG | 5216 |
| hs132678567 | CCAAAAGAAACACATTTCAATGG | 5217 |
| hs132682819 | CATTTAGTAACAATAAATGTTGG | 5218 |
| hs132681159 | ATAAGATTACCTTTCTGGTAGGG | 5219 |
| hs132680020 | TACAGAATCTAAAGATTGAAGGG | 5220 |
| hs132682235 | GTAAAGCTAGACGGATCAAAAGG | 5221 |
| hs132678217 | GTATAAATCCTGATACTAGCTGG | 5222 |
| hs132679124 | AATTGCAATGAAATGTATGTAGG | 5223 |
| hs132677920 | GCACAGATTAGTGTCACCTATGG | 5224 |
| hs132682790 | TTTTAGCACACCTGGTGCAGAGG | 5225 |
| hs132681041 | GCCCAAATCATCAAAGCAGATGG | 5226 |
| hs132682056 | TACTTGGAATGGATGAGCAAAGG | 5227 |
| hs132682984 | TGGGGTTACTAAACATTCAAAGG | 5228 |
| hs132680162 | AAAATCCACCCACCCCACTAGGG | 5229 |
| hs132677222 | ACCACATCAGACCGTGGGAACGG | 5230 |
| hs132680553 | ATTAGACCTCTATTTGTGCTAGG | 5231 |
| hs132681420 | TTAGATCTAATTTGTATAGGGGG | 5232 |
| hs132680815 | CCATTGTGTTTCCCATCCCAAGG | 5233 |
| hs132681068 | AAGTATCTCTCATAGGTCCTTGG | 5234 |
| hs132678492 | GTTTTGTCTTAAATTTCTGGAGG | 5235 |
| hs132681952 | TTACTGACGCGTCCCCCAAACGG | 5236 |
| hs132677297 | TTTTAACCTGAAAATCCAAAGGG | 5237 |
| hs132679848 | TGCTGTTCTCCCCAAAGCAGGGG | 5238 |
| hs132679146 | TTTTTATAATAAAAGCTGTAGGG | 5239 |
| hs132677384 | TCAAGTTGTTATATGTAAGTGGG | 5240 |
| hs132677623 | AAAGATTGCAGGTTGGAGGAGGG | 5241 |
| hs132679628 | GCTTTTTCAACTGGTAGTTGGGG | 5242 |
| hs132680664 | CTTGCTTAAGGAAAACTGGATGG | 5243 |
| hs132678887 | CTGCCTCCTATTATACCCTAGGG | 5244 |
| hs132678708 | CAGACAACTGAGTAACCAAATGG | 5245 |
| hs132678558 | TTTATGATGGATTAAGGGTGTGG | 5246 |
| hs132682743 | CAATGTCTGTGAATTGCCCAGGG | 5247 |
| hs132678111 | GGGGTATGCATGGGAGCAGAAGG | 5248 |
| hs132681640 | AATGTAATTTTTGCTAAGTGCGG | 5249 |
| hs132681222 | TTATTTCCTGCATTAGTCAAGGG | 5250 |
| hs132677846 | CTAAGAAGAGACTGAGTGCTGGG | 5251 |
| hs132681799 | AATCTAGGCAAGCTGCTACAAGG | 5252 |
| hs132677426 | ATAATTTCTCAATTACCCTACGG | 5253 |
| hs132680113 | ATTTAAAGTTAATTTTATAGGGG | 5254 |
| hs132680823 | GAGCCCAGAACATCTCTTTGTGG | 5255 |
| hs132679131 | AGGTAGGGTGGGGAAAAAGTGGG | 5256 |
| hs132680047 | AACATTTACCCATAACGTAAAGG | 5257 |
| hs132681486 | CATGTGCATTTGCAAGACCTTGG | 5258 |
| hs132677518 | TTAACCACCCAAAGCTGCTTAGG | 5259 |
| hs132678758 | ACAGGAGCAGAGCCTTGAGATGG | 5260 |
| hs132678481 | CAGTGCCAGGATTTGAAGGCGGG | 5261 |
| hs132677681 | ACTTAGTCTCAGAGTGGGAGTGG | 5262 |
| hs132682033 | ATATTAGTGTGAATGTATGTGGG | 5263 |
| hs132681430 | ATCATCTTGCAGATACAGACAGG | 5264 |
| hs132680503 | CAAGTAATCTGGACTATCACAGG | 5265 |
| hs132679127 | TGAAATGTATGTAGGTAGGGTGG | 5266 |
| hs132680199 | GTATCTGACTACCAGTTTCAGGG | 5267 |
| hs132677888 | GCCTTGAAGAAAGGTGTTGGTGG | 5268 |
| hs132678057 | ATCGTGTATGGGGAGAGTACAGG | 5269 |
| hs132681349 | TTGACAACTTACTTATTGTGTGG | 5270 |
| hs132681024 | ACATAATGCAAAATGAGCACTGG | 5271 |
| hs132683126 | TTCCCTTTATTAAGCCGGCATGG | 5272 |
| hs132680183 | GGGTGAAGAAAGGAGAGTCGTGG | 5273 |
| hs132679157 | GTGAAGCCAGGTAGTAAGGATGG | 5274 |
| hs132679125 | GCAATGAAATGTATGTAGGTAGG | 5275 |
| hs132680536 | TGAGAATCTATATTCTACATAGG | 5276 |
| hs132678458 | AGTGCTCAATGGGTTTTTGGAGG | 5277 |
| hs132680586 | TCAAATTCAAATAACGACATAGG | 5278 |
| hs132677401 | CAGAATGTTCAATTTTATAGGGG | 5279 |
| hs132677106 | AATACTTTCCAAAAAACGTCAGG | 5280 |
| hs132680152 | AGTTTCCGTACTCCCCTAGTGGG | 5281 |
| hs132678626 | GGCTGGGGAAGAATGATTTGAGG | 5282 |
| hs132677858 | AAGGTTATAGCAGTGTAGGCTGG | 5283 |
| hs132680612 | TGAGAGACTTAAAATTAGGTAGG | 5284 |
| hs132679133 | GGGAAAAAGTGGGAGAAGAAGGG | 5285 |
| hs132677946 | ACCACAGGGCTCTTGGACAAGGG | 5286 |
| hs132680523 | TAGTGATGCCCAATGAGCCTTGG | 5287 |
| hs132679595 | GATGTTTATATTCTGACGAAGGG | 5288 |
| hs132680888 | AGCTAGAAGGTAACTTTAGGAGG | 5289 |
| hs132682483 | GCTTTTAGATAGTAACAAGATGG | 5290 |
| hs132683186 | TATCTTTTGGTTTCACCAAGTGG | 5291 |
| hs132677228 | CCCTGTAGTATGAATACCCCAGG | 5292 |
| hs132678921 | CTCCAGTGATACTTTAACATGGG | 5293 |
| hs132680436 | GTGAGGCCAGTTTGCTAAAGGGG | 5294 |
| hs132678839 | TGTTGAAAACTATGTCCCATTGG | 5295 |
| hs132681869 | ATCAGTGGAAAGCACTTGAATGG | 5296 |
| hs132678642 | CACGTGGCTATACTGTTCATTGG | 5297 |
| hs132679703 | AGATAGAATTGAGAAGACTATGG | 5298 |
| hs132678529 | TAAAGGACTCAAACTTTTCGCGG | 5299 |
| hs132682540 | TAAATGGGAGCTTTGCCAAAGGG | 5300 |
| hs132682145 | TCAACATAATAGGACAGCCCAGG | 5301 |
| hs132682048 | AGTTTATCTTTCCAGTAAGAGGG | 5302 |
| hs132678414 | TTACATTCTCCATCCAGTCCAGG | 5303 |
| hs132679275 | CTTTCTGCTAACTTTTTGAAAGG | 5304 |
| hs132678773 | CTTTGATGTGTCACGTCCAGAGG | 5305 |
| hs132677059 | GGATGTCGAACCACTCTGACTGG | 5306 |
| hs132680100 | ACTGTATTCTGTAATTGCTAGGG | 5307 |
| hs132680285 | GCATCTATCTCTTTAGGACCAGG | 5308 |
| hs132682192 | AATTTCATCCTATTCTGTATGGG | 5309 |
| hs132678750 | GATATGCCATCCTATTAAGTAGG | 5310 |
| hs132678478 | TCATGTACAATTCATCCCATAGG | 5311 |
| hs132682081 | TGGTAATGTGTTTATAAGAGAGG | 5312 |
| hs132677717 | AATTCACTGTGTGTGATCCATGG | 5313 |
| hs132682022 | CTGAAATGTCTCTCTAGCACTGG | 5314 |
| hs132681472 | ATCATTAGCGTAGCTGATAGAGG | 5315 |
| hs132681775 | TCGTCCTCTCAGATGCCAAAAGG | 5316 |
| hs132678945 | CCCTCCTACCTAAAACTATGAGG | 5317 |
| hs132681271 | GCACACTCTTTATGGTGAAAGGG | 5318 |
| hs132677147 | GATCCATTTTCAAATTCCATGGG | 5319 |
| hs132679902 | AATTAACTTCAATAGGGCTGTGG | 5320 |
| hs132683075 | CTTCCTCAAACACAGGGAAAGGG | 5321 |
| hs132678347 | CCGTAATTGTTTCCCTTGAGGGG | 5322 |
| hs132678588 | CTCAATCCCCTCATATACAGAGG | 5323 |
| hs132680510 | ATAAACACTTTCATTTATCAGGG | 5324 |
| hs132678381 | GACCTAATAATTTCATGCGCTGG | 5325 |
| hs132681160 | AATAAGATTACCTTTCTGGTAGG | 5326 |
| hs132677613 | GTAAGCTTCGACCTCAGCCTTGG | 5327 |
| hs132682586 | GTACTTGAAATGTTACTGTCAGG | 5328 |
| hs132680895 | TGTGTGACGTTTTTGTAAAGTGG | 5329 |
| hs132678918 | GTGAAAACCCTCAAAAGAAAGGG | 5330 |
| hs132680069 | GTATTATCCATTTGCAGTAGAGG | 5331 |
| hs132682506 | TGAATCATTCTAATTGGTGAGGG | 5332 |
| hs132683085 | GCCTCTACAGCCATTTAAGTAGG | 5333 |
| hs132680737 | AGAACCTGATTTATTCAAAGAGG | 5334 |
| hs132682524 | TTAGAAATATGAAGAAATCAGGG | 5335 |
| hs132679225 | ACATTATATGCATAAGACATAGG | 5336 |
| hs132680351 | ATGTAAATAATCCGTGGGGGTGG | 5337 |
| hs132679308 | TTGACATCTGAATCTATGAAAGG | 5338 |
| hs132682326 | ATGCTCAGAGTGGATCTGATAGG | 5339 |
| hs132677970 | GTTGTGAATTATACTGCCAATGG | 5340 |
| hs132678637 | CTATGACGCGAGAGGATGGCAGG | 5341 |
| hs132678781 | GATTTGGTTCTGCATCAGGCTGG | 5342 |
| hs132678190 | CTAGGGCTTACCTTAGAGGCAGG | 5343 |
| hs132678477 | CATGTACAATTCATCCCATAGGG | 5344 |
| hs132680662 | TCATCCTCCCTCATAAGTATTGG | 5345 |
| hs132677925 | TATTTGATTTTAATTGGCAATGG | 5346 |
| hs132679059 | GGCACATGTTGCATAAAACTAGG | 5347 |
| hs132677928 | TCAACTAAGATAGACACAAAGGG | 5348 |
| hs132679149 | ACACAATCAGATTTATATTGTGG | 5349 |
| hs132678298 | CACAGCATTTTCTCAGGCATGGG | 5350 |
| hs132680240 | CTTCTGGCTCCCTTCCCTCAGGG | 5351 |
| hs132680605 | TCCCCATTTTATCATGTGAAGGG | 5352 |
| hs132682055 | TTGCACTTAGTATTATAATGAGG | 5353 |
| hs132677362 | TCTCTGGTGATGTCATTGTAAGG | 5354 |
| hs132678530 | AAAGGACTCAAACTTTTCGCGGG | 5355 |
| hs132681927 | ATAGTGTACTTCAATTCTGCTGG | 5356 |
| hs132678052 | AGGCTAATGTCTATTCAAATGGG | 5357 |
| hs132678019 | GGTAATTTGCCAGTAGGATGAGG | 5358 |
| hs132681577 | CATTTTGGAGGCAAGAGTTGAGG | 5359 |
| hs132682509 | AGTTTAAAAATCCAAACAATGGG | 5360 |
| hs132678233 | GTTCTGTAGATGTTCTATAGAGG | 5361 |
| hs132681518 | ATTAGATGATGAGACAAAATGGG | 5362 |
| hs132679747 | AGTCAGTGCTTTTAGTTTTGGGG | 5363 |
| hs132679967 | GTTACAGTCCAGTTACTTGAGGG | 5364 |
| hs132677411 | GGTTGCTTTTCTGACAGAAACGG | 5365 |
| hs132683079 | GGAAACTCTGTAAAGGGAGTAGG | 5366 |
| hs132681602 | ACTTTGGGCCGAAAATTTCAGGG | 5367 |
| hs132678272 | TGATTTCCAATCAGGTAGGCAGG | 5368 |
| hs132679943 | GTTCTCAGTATGTGACCTGCTGG | 5369 |
| hs132679764 | AAAAAATGTACATAATACTTGGG | 5370 |
| hs132682368 | CCCAGACAAAACCAGAATAGTGG | 5371 |
| hs132681716 | GGAGGAATTGGTCTTTCAGAAGG | 5372 |
| hs132682135 | TTAGAATCCAAGTTTACTCCCGG | 5373 |
| hs132680810 | ATTAAAATATTAAATGGGACAGG | 5374 |
| hs132680195 | TATCAGATCTGCTGGTTACAGGG | 5375 |
| hs132681305 | GCATAGTGAAACTGATGCCCTGG | 5376 |
| hs132679955 | TATTCTTTAAAGTTCCTTGGAGG | 5377 |
| hs132682824 | GAGCTAGAAGTTTTTATGGATGG | 5378 |
| hs132681249 | AGTTCTTGCTATCATTAAGAGGG | 5379 |
| hs132677989 | GTTTTCCTTCTGGCTGTGAGAGG | 5380 |
| hs132680848 | GGCAACCTATGGGATTAAGTAGG | 5381 |
| hs132679177 | AAGAGAAAGATTATAAGAAGAGG | 5382 |
| hs132680108 | TTTTGTTCACATGTAGGCTGAGG | 5383 |
| hs132678174 | AGATTTGATTCAACTGTCCATGG | 5384 |
| hs132681692 | AGCCACAGCACAACAAAAAGAGG | 5385 |
| hs132681424 | TTTCATGCCCATCAAATATGAGG | 5386 |
| hs132682734 | GCCCATTCTTCCTAATAGGCAGG | 5387 |
| hs132682530 | TTTCTTTAAGAACAGACTGTTGG | 5388 |
| hs132677620 | GAGGAGGGTGGGTCTATCCAAGG | 5389 |
| hs132679051 | GTTCTCAATAGCAAATTGCTTGG | 5390 |
| hs132682793 | TTTGTTTTCTGTGGTTGAAAGGG | 5391 |
| hs132683209 | AGCACTGCCTCAGAGCAATTCGG | 5392 |
| hs132681070 | GAATCCAAAGTATCTCTCATAGG | 5393 |
| hs132677801 | ACAGGGACAAGACCAAGAACAGG | 5394 |
| hs132682195 | GCTTTGATCCCATACAGAATAGG | 5395 |
| hs132679674 | TGTGCTGAAGTCTAAGACAAAGG | 5396 |
| hs132677855 | GCCTACACTGCTATAACCTTAGG | 5397 |
| hs132681130 | ATAAAAGTTTTAAAAAGCAAAGG | 5398 |
| hs132678831 | GAGCATTCTTGGGCACCAGCTGG | 5399 |
| hs132680104 | TATTGATGTAAACGTTATGCAGG | 5400 |
| hs132678696 | TTTATCAGACATTTTCTCCTGGG | 5401 |
| hs132683025 | GAGCAGCCTGCACAAGACTTTGG | 5402 |
| hs132681552 | TGACATACTCTCCAGCTGCTAGG | 5403 |
| hs132681281 | AGCAAAATGTCTTAACACTAGGG | 5404 |
| hs132681489 | GGTAGTGTTGGCTTTATACCAGG | 5405 |
| hs132681326 | ATATATTAGCATATGTTCTAGGG | 5406 |
| hs132680239 | GGGATAATACCCTGAGGGAAGGG | 5407 |
| hs132679944 | AGAACTGCATAGGCTTTGTATGG | 5408 |
| hs132679120 | AAGCATTTAGAGGGGAAGACTGG | 5409 |
| hs132680941 | GTGTTCTGCAGCAGGTTAAGTGG | 5410 |
| hs132678653 | TGATCCACATGATCTGGCCTCGG | 5411 |
| hs132677093 | TATGATTCAGTACCTAGCTCAGG | 5412 |
| hs132681802 | TAAGTCTTTGACCTTTCTAAGGG | 5413 |
| hs132677790 | TTCGACTTTGGAGAAGCAAAGGG | 5414 |
| hs132682206 | GTTTGAGAAGACATAGGGAATGG | 5415 |
| hs132678854 | GATATTTATATATTCGGCTGAGG | 5416 |
| hs132680575 | ATTCTCTTACCTTTTGAGAAGGG | 5417 |
| hs132677729 | GAATTGTATCTCTTCAGAAAGGG | 5418 |
| hs132681086 | ATACTTTTGGGTGGAGAGAGGGG | 5419 |
| hs132680036 | ATAACACTGTTAACTTACATGGG | 5420 |
| hs132678026 | TGGAAATTAAAATACAGTAAAGG | 5421 |
| hs132677496 | CAGAAGAAAAAGTTTAACTAGGG | 5422 |
| hs132677127 | TGACAAGACCTGTAAATGCCCGG | 5423 |
| hs132678942 | CCTCATAGTTTTAGGTAGGAGGG | 5424 |
| hs132677375 | GCTAGCTGCAAGGAATAACTTGG | 5425 |
| hs132678007 | CCAATGCTGTTTATCACCACAGG | 5426 |
| hs132682028 | GTCCTAGTCTTGCAAATGCTAGG | 5427 |
| hs132682783 | AAGTGCCTTTTCTATTCAGAAGG | 5428 |
| hs132681230 | TACCATAAAAAACTTCTAAGTGG | 5429 |
| hs132678676 | GAAGTTTGCCAAATAATACTTGG | 5430 |
| hs132680570 | TTAAGACATTATTAAAGTGTGGG | 5431 |
| hs132677050 | GCAAAAGACCACAATCTACCTGG | 5432 |
| hs132677248 | CTATTGACTTGCACTCCCACAGG | 5433 |
| hs132683120 | TCTCCCAATGAGTGATATGATGG | 5434 |
| hs132681890 | TTTGAACCTGAATCAGATGATGG | 5435 |
| hs132677305 | CTTAGCGACAGTTATGTCCATGG | 5436 |
| hs132677662 | ATATATAGCTTCCAAGAATGAGG | 5437 |
| hs132680252 | CTTACACTTGCCATTGGTCGGGG | 5438 |
| hs132680885 | ATTCGCAGGTGGTACTTGGTTGG | 5439 |
| hs132679791 | TTTTATAACCAGTTGACAGATGG | 5440 |
| hs132678528 | CTAAGGTTAATTTCAACGAAAGG | 5441 |
| hs132683282 | ACACTATGAGAGAAATAAAACGG | 5442 |
| hs132679466 | TTTTCCCCCTAGTTTAAAAGTGG | 5443 |
| hs132677207 | GTGCCCCAGCTAGGTGTACATGG | 5444 |
| hs132683200 | GATCAGTTATAACAGACTTAGGG | 5445 |
| hs132678193 | CAGGTAAAGAAAACAATCTAGGG | 5446 |
| hs132681487 | CTAACAAGCTAGCAAAATCCTGG | 5447 |
| hs132683236 | TTACAAAATCAGACCCATAGAGG | 5448 |
| hs132678843 | TGTATTGCCTTATATACACCTGG | 5449 |
| hs132683187 | TTGATTCAGTTTAAGCCACTTGG | 5450 |
| hs132679193 | AGAGACTCCACTCTTTTGCTAGG | 5451 |
| hs132681896 | AACATTGATAAATTAAACATTGG | 5452 |
| hs132680249 | ATAATCCTTACACTTGCCATTGG | 5453 |
| hs132682605 | ATTTCACTTGGCCTATCTCAGGG | 5454 |
| hs132682462 | ACCTTTTCTAGGATAAAGCTTGG | 5455 |
| hs132678902 | TAGAGCTATTTTTAAAGCAGTGG | 5456 |
| hs132680203 | GCAAAGTGATCTCTGCCAGCTGG | 5457 |
| hs132677242 | TATTAGTTTGGCATCCCTGTGGG | 5458 |
| hs132677909 | AAAAAAATATCTTTCAGCCAGGG | 5459 |
| hs132681769 | CATCTGAGAGGACGACAGCTAGG | 5460 |
| hs132677671 | CATTGGTGCTCAATATACTAAGG | 5461 |
| hs132680886 | GCGAATTGGACAAAATTCAATGG | 5462 |
| hs132681247 | TATCATTAAGAGGGGACAAATGG | 5463 |
| hs132679118 | AATGTAACTAAGCATTTAGAGGG | 5464 |
| hs132678834 | AATGGGAGTGTCTGACCAGCTGG | 5465 |
| hs132680085 | CTTGAGTTAGGTGTAAAGAATGG | 5466 |
| hs132682600 | CTTCTTCCGTGGCAATATGCAGG | 5467 |
| hs132682115 | GGGCAACTAATTCTGAAAACGGG | 5468 |
| hs132678987 | ACTCTTGTGGGTGGCAGTAAGGG | 5469 |
| hs132680581 | GCACACACTCCCTTCTCAAAAGG | 5470 |
| hs132678167 | AAAGTAGTAGAAAACAATAGTGG | 5471 |
| hs132677541 | TCTTTTTTGCAAGATTGCATTGG | 5472 |
| hs132681301 | CCTGTACTTGATATGCTTGGAGG | 5473 |
| hs132677669 | ACTAAGGATTTTTCAATGCAGGG | 5474 |
| hs132678888 | CCTGCCTCCTATTATACCCTAGG | 5475 |
| hs132679470 | CAGGGGCCACTTTTAAACTAGGG | 5476 |
| hs132682996 | TGATAAGTCCTATGAGAAGCAGG | 5477 |
| hs132680731 | GGTAGAGAGTTTCTTTCCCAAGG | 5478 |
| hs132678509 | ACCTAAACTGCAAAGCAAACCGG | 5479 |
| hs132679034 | GGTCAAAATGTCACACCAAAAGG | 5480 |
| hs132682777 | TGAGCAAGAAGGTAACCAAAAGG | 5481 |
| hs132678726 | GCAAATTTAATCACCTCTGAGGG | 5482 |
| hs132680296 | CTCAACCTAATTTATTTCTGCGG | 5483 |
| hs132679274 | TCAAATTCTATGAGGTAGAGAGG | 5484 |
| hs132679391 | TGTATTTTGTCTTTGTGCAAAGG | 5485 |
| hs132680716 | ATAAAACTGAATCCTTAAGCTGG | 5486 |
| hs132678136 | TTAGCTAACTTGCTTTACCTTGG | 5487 |
| hs132678256 | AGGAAATAGTAGGGTGACATAGG | 5488 |
| hs132677870 | CTTTAATCCAGTGCCAGGCCCGG | 5489 |
| hs132681224 | TAGAGACCCTTGACTAATGCAGG | 5490 |
| hs132678108 | AAGAAGAGACGAATTTTGCTTGG | 5491 |
| hs132678514 | GCTCTTGTCTCAAGATGACTTGG | 5492 |
| hs132680881 | GCGTCTTGCCTACCTGTCTCCGG | 5493 |
| hs132679812 | ATGTATGTATGGTACATGTAAGG | 5494 |
| hs132681548 | GACAGGTCATCCCTAGCAGCTGG | 5495 |
| hs132681482 | ATTTGCATCATTGTCCCTGCAGG | 5496 |
| hs132677470 | TTTCTTCTATCATTTCCAAAAGG | 5497 |
| hs132680297 | TTGAGCAAGACTACACCTAATGG | 5498 |
| hs132683159 | TATACATTCCTGCCTTTGATGGG | 5499 |
| hs132679987 | AATATCCAAAAGTTAATCAATGG | 5500 |
| hs132682837 | TTATTTTCAATGATTTTCCATGG | 5501 |
| hs132680601 | TTTGGGAGAAGAAATTCAGTAGG | 5502 |
| hs132680879 | AATTCAGAAGGACCGGAGACAGG | 5503 |
| hs132677412 | CTTTTCTGACAGAAACGGTATGG | 5504 |
| hs132682650 | GAGTGAGTAGTATGAGCAAATGG | 5505 |
| hs132681512 | GGGATGAAAGAGGAGACAAAGGG | 5506 |
| hs132679898 | GGAACTCAGGTAAAACTAGTAGG | 5507 |
| hs132679849 | ATGCTGTTCTCCCCAAAGCAGGG | 5508 |
| hs132679700 | CTCAGACTTAGTCATCTAAAGGG | 5509 |
| hs132678001 | CAGACTGTTCTTGGGCCGCTGGG | 5510 |
| hs132680819 | TTTCAGGGACATATTCAGAAAGG | 5511 |
| hs132678106 | AGACGAATTTTGCTTGGCTAGGG | 5512 |
| hs132680687 | CTTAATTTTGTAATTTCCCAGGG | 5513 |
| hs132677414 | CTGTCAGAAAAGCAACCATTGGG | 5514 |
| hs132679153 | TGGAATAGACTAAACTGCACTGG | 5515 |
| hs132679163 | CCTTATCCATCCTTACTACCTGG | 5516 |
| hs132682126 | TTACCAAGTTTCCATTTACAAGG | 5517 |
| hs132682701 | GATGTACTATTTTTGAAGCCAGG | 5518 |
| hs132682742 | GCAATGTCTGTGAATTGCCCAGG | 5519 |
| hs132678485 | AAGACAAAACCCCAGAGAAAGGG | 5520 |
| hs132680634 | AAACACTCAGAGTAGACAACAGG | 5521 |
| hs132677121 | ACTTCCACGATTAAAAACATAGG | 5522 |
| hs132678443 | CATGCTCACGATTCTTGCCTGGG | 5523 |
| hs132677977 | ACTCTTTTCATGATTAGAGATGG | 5524 |
| hs132682419 | ATCTCTTTCCCTTCAAGAGAGGG | 5525 |
| hs132681393 | GGAGTCCTATAGGATAATTTGGG | 5526 |
| hs132680564 | TGTAAAGCTGGACTTCAAGTTGG | 5527 |
| hs132681303 | ATATTACTGCAGTACAAGCCAGG | 5528 |
| hs132679142 | GGCATTTTGTGCAATGCTGAGGG | 5529 |
| hs132681551 | GACATACTCTCCAGCTGCTAGGG | 5530 |
| hs132677761 | CCTGCTTTGCTCAAATGTCAGGG | 5531 |
| hs132679609 | CATCTTTAAAAAGTTTCTATGGG | 5532 |
| hs132677817 | TCGCCTGGCTTGTTTGTGCAAGG | 5533 |
| hs132679060 | TCATAAAATGTAATATCTGTTGG | 5534 |
| hs132681066 | GGTCCTTGGAGCAATCGTATTGG | 5535 |
| hs132682607 | GGTCTATTCATAGACTTGTATGG | 5536 |
| hs132679573 | GTATTCTTATTCCCATTATGTGG | 5537 |
| hs132680076 | AGTATTCTACTTAGAGAAAGAGG | 5538 |
| hs132679422 | ATTCCTTTACACTCTAAAAGTGG | 5539 |
| hs132681437 | GAGTCCCAAGTACACCTTGATGG | 5540 |
| hs132678010 | TTGACTTTGCCCTTGCCAAAAGG | 5541 |
| hs132681680 | GTGACATACAAAAAGTCTACTGG | 5542 |
| hs132677968 | ATTGTGTTAATGGATTATAATGG | 5543 |
| hs132679665 | GATCAGCCTTTGGTAGAGATAGG | 5544 |
| hs132679860 | TCTCTTTCTAACGGTAAAAGTGG | 5545 |
| hs132677145 | TTTTTGTCTAAGTATATATGTGG | 5546 |
| hs132681027 | TTACGTATGTATGACTTGCTTGG | 5547 |
| hs132680714 | TGTTGTACCTCAAATAGCTTAGG | 5548 |
| hs132682247 | TGGCATGTCACCTAACTGGCTGG | 5549 |
| hs132683125 | TGTACTTCCCTTTATTAAGCCGG | 5550 |
| hs132682696 | TACTGCTTTGGAGTATTGAAAGG | 5551 |
| hs132683197 | TCTATTATCAAGCATATCCTAGG | 5552 |
| hs132682198 | TTAAAAAGCACCTAGAGGATTGG | 5553 |
| hs132680825 | CTTTGTGGATATGTGACTAAAGG | 5554 |
| hs132680796 | CACAGAAAGTTGGAACCAAAGGG | 5555 |
| hs132679827 | TTCTGCTTATGTGTAAGCCTTGG | 5556 |
| hs132679841 | ACTTAGAGAGTATACATTGAAGG | 5557 |
| hs132680408 | TGCCTAGTCCCAATTTCTGGTGG | 5558 |
| hs132677049 | TCAAGGCAGTTACGGGTAAAGGG | 5559 |
| hs132682333 | TAGGATTAGCTTATCCCAAAGGG | 5560 |
| hs132678875 | GACTGCTCTGGGTTGTACTAAGG | 5561 |
| hs132677176 | ACATGAATCCCAAAGCCATCAGG | 5562 |
| hs132681168 | TGTTTTTTCTCCTAAATAGCAGG | 5563 |
| hs132682420 | TTTCCCTTCAAGAGAGGGAGTGG | 5564 |
| hs132677269 | CATGAATTCCTCCTTGGGCCAGG | 5565 |
| hs132682699 | TGAAAGGAGTTTTAGTATAGGGG | 5566 |
| hs132682953 | GAGACCACTCCAGAGAATATAGG | 5567 |
| hs132678948 | ATAATACAAGTCGAAAAAGTTGG | 5568 |
| hs132678908 | GTTGCTCCAAATTTTAACTTGGG | 5569 |
| hs132680043 | ACAAACAAATATAATGAAAACGG | 5570 |
| hs132680390 | TTTATTTCAAGAAAAAAAGCAGG | 5571 |
| hs132682369 | ATGATTTGCTTTCATCATGATGG | 5572 |
| hs132679458 | AGAAACCCAGCAAGCTGAAAGGG | 5573 |
| hs132683034 | GATCTATGTAAAACCTTCCATGG | 5574 |
| hs132679447 | AAATGAGTGAATACATAAAACGG | 5575 |
| hs132679329 | TTGCATCTCACTAGGCAAATGGG | 5576 |
| hs132683189 | TAGACACTTTTGAAGAAGATGGG | 5577 |
| hs132681679 | TTATGGGATAAAGAAACATATGG | 5578 |
| hs132680445 | CATACTAGATTAACTTGAAAGGG | 5579 |
| hs132678337 | TCATACACATTTTTAAAAGCAGG | 5580 |
| hs132679285 | GCTCTGCTGCATGGTAGAACAGG | 5581 |
| hs132680878 | TTTTATGAATTCAGAAGGACCGG | 5582 |
| hs132681615 | GAAAACATGTTATATGAGAATGG | 5583 |
| hs132677276 | CCACTCAGTTCACTTGATAAGGG | 5584 |
| hs132678483 | GATACAGTGCCAGGATTTGAAGG | 5585 |
| hs132680957 | ATATTTCTGAGGACTGGGGGTGG | 5586 |
| hs132677175 | GGGAAAGGTGAGAGAAGTCTTGG | 5587 |
| hs132679479 | ATCAAACTGTGAATTTAAAGTGG | 5588 |
| hs132680207 | TGTGAATCCCCAGCTCCAGCTGG | 5589 |
| hs132682598 | ATATTGCCACGGAAGAAGGCTGG | 5590 |
| hs132678266 | CTGGGCCTGCTGTATTCCCTGGG | 5591 |
| hs132682795 | CATTTGTTGTTTGTTTTCTGTGG | 5592 |
| hs132677461 | TTGCAACTGCTGTTTAAGCTGGG | 5593 |
| hs132681316 | TCATGGATCTACACAACCTCTGG | 5594 |
| hs132677105 | TGGGAGCTCTCACAAGCCTTGGG | 5595 |
| hs132678191 | GATTGTTTTCTTTACCTGCAGGG | 5596 |
| hs132681382 | GAGAGTGAGAACGAGGATGAGGG | 5597 |
| hs132677818 | AGCCAGGCGAGAGAGAAAACTGG | 5598 |
| hs132678126 | AGTTATTTATGAATACAAACAGG | 5599 |
| hs132681151 | ATTAAAGTCAGCTTTGCAAATGG | 5600 |
| hs132678144 | ACATCCAGATATTCAATAGTAGG | 5601 |
| hs132681933 | ATACCTGGAAGATATATAATGGG | 5602 |
| hs132678743 | CTCATTCCTACTTAATAGGATGG | 5603 |
| hs132677596 | ATCATTATCAGCTTGAAAAGTGG | 5604 |
| hs132681706 | ATTGTTTGCTACCAGGAGAAAGG | 5605 |
| hs132677912 | AGACAATGCTAGTTTTAGTAGGG | 5606 |
| hs132679597 | TCAGGTGTGATACTGTAAACAGG | 5607 |
| hs132677697 | CTTCAAGAGAGGATGACTATAGG | 5608 |
| hs132679565 | TTCTGGCCTTAAGTTCAAAAGGG | 5609 |
| hs132680830 | TTGCCTCTGCACCTTTACACTGG | 5610 |
| hs132679316 | TTTAAAATATCAACTACCTCAGG | 5611 |
| hs132681240 | GTATGCTAATTAAAATATACAGG | 5612 |
| hs132677394 | CATTTTACCAGGTATTCTAAAGG | 5613 |
| hs132681093 | GATGGTATATATATAATCCTTGG | 5614 |
| hs132677281 | TTTTAGGTCCTTTCGTGTTGTGG | 5615 |
| hs132680192 | ATGTACAAGCCCGAAAAGGCTGG | 5616 |
| hs132679683 | GACCCTTGCAATAGATAAGTTGG | 5617 |
| hs132678454 | CCAGCAAACACAGTGCTCAATGG | 5618 |
| hs132679105 | TGTATCATGCATACTAAAGATGG | 5619 |
| hs132680084 | TCTCTATGACACACTATACTAGG | 5620 |
| hs132682558 | CGCTGTTTCCCGAAATGCCAAGG | 5621 |
| hs132678199 | GATCAAGAATGGATGGAAACAGG | 5622 |
| hs132682281 | ACAAGTAGCAGGGTAGAATCAGG | 5623 |
| hs132682066 | AATTTACTAAGTTATTTGAATGG | 5624 |
| hs132679279 | GGCATGTTTGGTCTTGCAAAGGG | 5625 |
| hs132682534 | TTAGACCAACGCATTGTAGTTGG | 5626 |
| hs132677768 | CAGCTTTTAAAAGGAGGAGGGGG | 5627 |
| hs132680009 | TACTAAACACAGACAAATGATGG | 5628 |
| hs132679903 | CACAGAAATTAACTTCAATAGGG | 5629 |
| hs132677232 | AACATACTATTTCTCTTGGATGG | 5630 |
| hs132680722 | CTCACTGTCAATTTTGAGTGGGG | 5631 |
| hs132680569 | GTTAAGACATTATTAAAGTGTGG | 5632 |
| hs132680940 | GGAATAACAAATGAATCATAAGG | 5633 |
| hs132680860 | AGTGATCTCCCTAAGCATACAGG | 5634 |
| hs132678873 | TCTGGGTTGTACTAAGGAGTAGG | 5635 |
| hs132680521 | GGCATCACTAGGAATACAACAGG | 5636 |
| hs132679022 | AACACTAATCAAAAGCAAATCGG | 5637 |
| hs132677672 | TCTATCTCGTAAATAAGAGCTGG | 5638 |
| hs132679937 | ATTTTTCCAAATGACTAAGTAGG | 5639 |
| hs132683231 | AAAATGTTTGTCATAGAAAAGGG | 5640 |
| hs132679440 | AACACTAAACCAGTTTCAAAAGG | 5641 |
| hs132678442 | TGAGCATGTGTCTTAGCAAATGG | 5642 |
| hs132678779 | GCATCAGGCTGGTAGTTGCTAGG | 5643 |
| hs132680749 | TGTAGAGAAATAAGAGGAACCGG | 5644 |
| hs132680767 | TGGAGAAGCAAGAGAAGCTAAGG | 5645 |
| hs132681428 | TGCAGCTCCTCATATTTGATGGG | 5646 |
| hs132681910 | CCAAAATTCCATTGGAATGACGG | 5647 |
| hs132682924 | TGTCTATTGCCAAATGTAAAGGG | 5648 |
| hs132680987 | TGAGCCACTTTATTGTTGATGGG | 5649 |
| hs132680715 | ATTGCACTGCACATTGCCTTCGG | 5650 |
| hs132678968 | GCCAAAATAAAGGGGATGTCAGG | 5651 |
| hs132678583 | TGGAGTACAGTTCAAAAATAGGG | 5652 |
| hs132680277 | GCTGAAATGAGACACGTTGCCGG | 5653 |
| hs132682027 | TCCTAGTCTTGCAAATGCTAGGG | 5654 |
| hs132680138 | CTATTGTAGCCATATCAATAAGG | 5655 |
| hs132681988 | CCATCTACCCTGATTTTTGGAGG | 5656 |
| hs132682897 | TTTTGTGTTTCAGGGCAGATAGG | 5657 |
| hs132678441 | GCCTGGGTCTGACATAACTTTGG | 5658 |
| hs132682136 | AGGTTCTCCGGGAGTAAACTTGG | 5659 |
| hs132677113 | TGCTCTTTACATACACTCCTTGG | 5660 |
| hs132679758 | ATCCATCTAGTGTTTGATTGAGG | 5661 |
| hs132677960 | TTTTGGCTATAACCTCTAAAGGG | 5662 |
| hs132678375 | GTGTCCTTATAGCTTCTTGGTGG | 5663 |
| hs132679299 | TTTACGTTCAAATGTTGTAAAGG | 5664 |
| hs132682507 | TCATTTGCATCACTGCTGCTTGG | 5665 |
| hs132680137 | ACTGATCTATCAGCCAAAATCGG | 5666 |
| hs132680118 | CTATGTTACACTCAGTTAGCTGG | 5667 |
| hs132682892 | TTTCACTGCAATCCTTTCATGGG | 5668 |
| hs132680435 | TGTGAGGCCAGTTTGCTAAAGGG | 5669 |
| hs132678703 | ATAGATCAGCTGCTACACTATGG | 5670 |
| hs132681359 | GGTGATCTACAAGAGAAGTTGGG | 5671 |
| hs132677933 | AATAATTGCTAGAGGGAAAAGGG | 5672 |
| hs132679713 | TGATCAAAAAAGTATAAAACAGG | 5673 |
| hs132679354 | TTAAACTACATTGAATTCCCTGG | 5674 |
| hs132677957 | ATTCAGGTAAGTGCTTAATTGGG | 5675 |
| hs132678301 | ATAAGACACAGCATTTTCTCAGG | 5676 |
| hs132680535 | GAGAATCTATATTCTACATAGGG | 5677 |
| hs132679641 | TTGTATGGATTTTATCTGATGGG | 5678 |
| hs132679397 | GTTGTGCTTTGGTTTACCTTGGG | 5679 |
| hs132679209 | AATCATAGATTCTTTGCTAAGGG | 5680 |
| hs132682250 | TTAGGAAGCACCAGCCAGTTAGG | 5681 |
| hs132677758 | CATGTGTCCTGAGGACCTACAGG | 5682 |
| hs132682237 | GCTGACGCTGACACGGGCATTGG | 5683 |
| hs132682675 | TTGAAGACTCCCTTTTAAGTTGG | 5684 |
| hs132679006 | TATTATACTAAATTAAAAACAGG | 5685 |
| hs132679913 | CTTCAGTGTGTACAGTAATAGGG | 5686 |
| hs132682225 | TTTTGCCTGGATAATTCAAATGG | 5687 |
| hs132682844 | AACAGGGCCTTATGATTAAAGGG | 5688 |
| hs132679862 | GTATTTTGTACATGAGGAAAAGG | 5689 |
| hs132680116 | CTGAGTGTAACATAGAATATAGG | 5690 |
| hs132682531 | TTAAGAACAGACTGTTGGCCAGG | 5691 |
| hs132679864 | AATTATCAAACCCCATTATGAGG | 5692 |
| hs132683127 | ATCCATGCCGGCTTAATAAAGGG | 5693 |
| hs132680571 | CTTAACTAATCACATTGCTTGGG | 5694 |
| hs132682216 | CTGTTTCTAGAACTATATTGTGG | 5695 |
| hs132681815 | GACAAAGTTCCTTATTTGCTCGG | 5696 |
| hs132677136 | TGTTCCCTGCTTGAAGTACCCGG | 5697 |
| hs132682933 | CTGTGAAGCTCTAAAACAACAGG | 5698 |
| hs132683080 | ACATTTGGAAACTCTGTAAAGGG | 5699 |
| hs132678231 | TATTAAGAATATCATGAAAAGGG | 5700 |
| hs132682677 | CATTCATTGCCAACTTAAAAGGG | 5701 |
| hs132677158 | TTGTTTTTCAAATTGAATGAAGG | 5702 |
| hs132681578 | CAAGAATGAATGCATTTTGGAGG | 5703 |
| hs132679732 | TTTTCTTCATCACCAAATACAGG | 5704 |
| hs132677499 | AAAGTCTTCCATGTGAGATTCGG | 5705 |
| hs132681133 | ATCTCTGTATCTTCAGGCACTGG | 5706 |
| hs132677609 | TAGACTGTAATATCCAATATGGG | 5707 |
| hs132679804 | TGTACTATGTGCAATTAACTAGG | 5708 |
| hs132678956 | TCTACTAATCCATTGAACTAAGG | 5709 |
| hs132681376 | GTGATGCCAAAACAAACAACTGG | 5710 |
| hs132680808 | TTTAATCTGAAGAAATCCTTGGG | 5711 |
| hs132681508 | TATGTTCCTGGTTCAGGAAACGG | 5712 |
| hs132677730 | TGAATTGTATCTCTTCAGAAAGG | 5713 |
| hs132677065 | GAGTTATCCAGTATGAGAATAGG | 5714 |
| hs132678321 | TTATCTTTGATTTCGATTAGAGG | 5715 |
| hs132682908 | AAAAGGTCATTCCCCTTTGAGGG | 5716 |
| hs132683266 | AAATATGCTCTAAACTATAGTGG | 5717 |
| hs132681827 | CTTCAGTGGGTCAAATCCCATGG | 5718 |
| hs132680727 | CTAAAACCCCTCTTGGCCTTGGG | 5719 |
| hs132682805 | AATAATTTGAATTAAAAAAACGG | 5720 |
| hs132681477 | GCTTGACTTAGCTCTGTGATTGG | 5721 |
| hs132682722 | GTTCTGGCAGGGACTGAAGTTGG | 5722 |
| hs132677917 | TGACACTAATCTGTGCCAAATGG | 5723 |
| hs132681882 | TGATCTCCAACTCTGACAATGGG | 5724 |
| hs132679264 | ATACTGTTAAACACAAAATCAGG | 5725 |
| hs132677706 | TTAAAATGTTCAATGGTAGTTGG | 5726 |
| hs132679286 | GCTGCATATTATATTTTCCATGG | 5727 |
| hs132678847 | CAATTCTTCTTGCTAAGGCCTGG | 5728 |
| hs132683243 | TATGCCACTCTTAAAGCCTTGGG | 5729 |
| hs132682859 | CTTAGTTTGCACTCATCACCTGG | 5730 |
| hs132677995 | CTTTTCAAATTTTTACCAAAAGG | 5731 |
| hs132682044 | ATCTGCATTTAATCAACCCTGGG | 5732 |
| hs132679725 | TAGATAATCTACAGAACAAATGG | 5733 |
| hs132678296 | ATTTGTGCTTGTCATTAAGCTGG | 5734 |
| hs132677863 | CTGCTCTTTTCCCTTCTGTAGGG | 5735 |
| hs132678032 | TCTTTTCCAGTTCAACAGGCTGG | 5736 |
| hs132681839 | ATTATCTTTGAAGAACTCTTGGG | 5737 |
| hs132679523 | GTACTCTTGCTCTCAAGCTCTGG | 5738 |
| hs132682989 | CCCCCCTGCCTGCTTCTCATAGG | 5739 |
| hs132679398 | TGTTGTGCTTTGGTTTACCTTGG | 5740 |
| hs132681279 | CTCAAACCTGAGTAATAAACAGG | 5741 |
| hs132679300 | GAGATCCTGTATCGTCATATTGG | 5742 |
| hs132680971 | TTAATATCAGAGTTACCCTTAGG | 5743 |
| hs132682455 | TCTGTTACCATTACTGCTGTGGG | 5744 |
| hs132680281 | GATAGATGCACATCAACTTAAGG | 5745 |
| hs132681038 | CTTCTCAAGCTCCTTGTAAGTGG | 5746 |
| hs132681524 | CTACACTATAAGATAAATGTAGG | 5747 |
| hs132678819 | TTGGGCGTCAAGGTTGATAAGGG | 5748 |
| hs132682188 | TGTGCACACACAACACATTACGG | 5749 |
| hs132680786 | GCTTGCCAGTTTTTTTCAGTGGG | 5750 |
| hs132677473 | TTAGGAGCAATTTTATTGGCAGG | 5751 |
| hs132678015 | CTATGCCCTCCTCATCCTACTGG | 5752 |
| hs132682929 | GCATGAAGCATTAGTGGTGGTGG | 5753 |
| hs132681401 | TGAAGCCAAGATGAGGTAAAGGG | 5754 |
| hs132678625 | AGGAACCTCAAATCAGACTATGG | 5755 |
| hs132678476 | GATGAATTGTACATGAAATCAGG | 5756 |
| hs132680590 | TAAATTGCCATGGAAATCATAGG | 5757 |
| hs132682120 | CTTATACAGATGTAGAGCTTAGG | 5758 |
| hs132680329 | ATCTCTCCCCCTTAGACAACCGG | 5759 |
| hs132680793 | TGAACGATGGTGCTCCCCTTTGG | 5760 |
| hs132677442 | TTCTTCTTCCATGCCTGTAAGGG | 5761 |
| hs132677641 | TCATGTTTGTTTACCTGCAATGG | 5762 |
| hs132677420 | TTTCTTCTCTGCAATTTCCCTGG | 5763 |
| hs132677237 | AATTCTATAGAGTAAGTTGCAGG | 5764 |
| hs132677283 | TAGTATGAAATAAAGAAATACGG | 5765 |
| hs132677410 | TTAGCCTTCATTTCCATCACTGG | 5766 |
| hs132682118 | TTTCAGAATTAGTTGCCCTATGG | 5767 |
| hs132680056 | ATACACACAGGCAATCAATATGG | 5768 |
| hs132677536 | TATTTCTCATAAAAACTCCTAGG | 5769 |
| hs132680764 | CCTACCTTAGACTAGGCATATGG | 5770 |
| hs132679603 | GTAAATTTAATGATGTCTAAAGG | 5771 |
| hs132677182 | AAGACACAAGGACTACGTTTGGG | 5772 |
| hs132679958 | GTAACAAGATCTGAATGTTTGGG | 5773 |
| hs132683242 | CTATGCCACTCTTAAAGCCTTGG | 5774 |
| hs132682372 | TCTTCATTCTTTTCGTGTGAAGG | 5775 |
| hs132682830 | ATTTACAAGTTAACGTTCACTGG | 5776 |
| hs132677650 | CTTAAGTATGGGGAGGGCGGGGG | 5777 |
| hs132681018 | TCAATCTGAAATTTAACATAAGG | 5778 |
| hs132678089 | AATAGGCTGAAAGCTCCAACAGG | 5779 |
| hs132680938 | ACAAATGAATCATAAGGTAGAGG | 5780 |
| hs132680724 | CTCTCACTGTCAATTTTGAGTGG | 5781 |
| hs132682425 | ATGCTAATCCAAAATAATAAGGG | 5782 |
| hs132680045 | GTTTAAACAGAACAAGGAATTGG | 5783 |
| hs132677149 | GTGTTATGTTCTTTTTCCCATGG | 5784 |
| hs132680181 | TATTTGAATAAATATCATAAGGG | 5785 |
| hs132678928 | AGGAACAAGCACGATTTTAAGGG | 5786 |
| hs132678360 | ATATGTTGTTATACAGTCATTGG | 5787 |
| hs132682851 | CTACGGAGAGTACTTTTCATAGG | 5788 |
| hs132682207 | ACAGTTCACTGAAACTTAAAGGG | 5789 |
| hs132683213 | TGAGTTGTGATAAATGATAATGG | 5790 |
| hs132681049 | TTACATTCATTGTAACCTCTTGG | 5791 |
| hs132679339 | ATAAAAACAAAACAGTTAGATGG | 5792 |
| hs132678244 | GTCTTAGTATAGGGGACATTGGG | 5793 |
| hs132680421 | GTACATAGTCACTTTATAAGAGG | 5794 |
| hs132681149 | TAACTTAGAGATATAATAAGTGG | 5795 |
| hs132680414 | GTTTTATCCCACCAGAAATTGGG | 5796 |
| hs132683093 | CATTACATAATGACAAAAATGGG | 5797 |
| hs132678342 | AATGTAGAAGCGAAATTTGAGGG | 5798 |
| hs132678923 | GCTGTTATTCAGGTGCTTGAAGG | 5799 |
| hs132681880 | TTATAATTTACAAGTTCTTAGGG | 5800 |
| hs132682988 | GACAGTGAAGACTCAATAACAGG | 5801 |
| hs132682732 | TCCTGCCTATTAGGAAGAATGGG | 5802 |
| hs132679912 | AGTATCCACACACATTTCATTGG | 5803 |
| hs132680454 | AGTATGTTATCCTCAGGCTTTGG | 5804 |
| hs132679297 | TACAACATTTGAACGTAAAACGG | 5805 |
| hs132682723 | TCCTTTTGCTTGTTCTGGCAGGG | 5806 |
| hs132679270 | GTAGAGAGGGATTTGGGCTGAGG | 5807 |
| hs132677061 | GCTATGACCTATTCTCATACTGG | 5808 |
| hs132677474 | ATTGTAGGTCCCATTTGTAATGG | 5809 |
| hs132679692 | CAAAAAATAGAGAACCTTGTAGG | 5810 |
| hs132683223 | ATAACTTTCCTAAATATCCTAGG | 5811 |
| hs132683274 | CCCTCATATCTTCTTCTGCTGGG | 5812 |
| hs132679541 | TCTGTTTTTGATGTTGCAGATGG | 5813 |
| hs132682686 | CTTTTTCCATATCATTTCCTGGG | 5814 |
| hs132683060 | ATATATTGTCCCAACTGAACAGG | 5815 |
| hs132682601 | GGGATCCTGTACCCTGAGATAGG | 5816 |
| hs132678448 | AGGCACTTCAGCAGGATATAGGG | 5817 |
| hs132677070 | TTTGCTCAGGAATTATACTAAGG | 5818 |
| hs132678615 | GCTAACAATAAAATACTAATGGG | 5819 |
| hs132679179 | GGTGTAGTAGAGAGGAAGATAGG | 5820 |
| hs132677999 | GACTGCGGCAGACTGTTCTTGGG | 5821 |
| hs132679190 | AAGGTTTTGAGCAGAATAAAGGG | 5822 |
| hs132680936 | TGATTCATTTGTTATTCCTAGGG | 5823 |
| hs132678958 | TCTCCACTTTCCGCAGCAAATGG | 5824 |
| hs132679532 | GACAAGCAAGAGATAAATATCGG | 5825 |
| hs132677047 | GGCATGATCAAGGCAGTTACGGG | 5826 |
| hs132677898 | GGAGGGACTTTTTTTGGAGTTGG | 5827 |
| hs132680298 | CCTAATTTATTTCTGCGGCTTGG | 5828 |
| hs132677633 | AAGACACTGAAGAAAGAATAGGG | 5829 |
| hs132681909 | CCTTTTGTCCAAAATTCCATTGG | 5830 |
| hs132680532 | TTAAATCTAATACAGTCGTTTGG | 5831 |
| hs132677244 | TGTGGGAGTGCAAGTCAATAGGG | 5832 |
| hs132681368 | TGTATGACCTTTATACACTTTGG | 5833 |
| hs132683118 | AAAACCATCATATCACTCATTGG | 5834 |
| hs132678561 | CCAGGCATCACTGTTTATGATGG | 5835 |
| hs132680723 | TCTCACTGTCAATTTTGAGTGGG | 5836 |
| hs132682927 | AATTTTGCATGAAGCATTAGTGG | 5837 |
| hs132680606 | TGATAAAATGGGGATTTTAGTGG | 5838 |
| hs132680812 | TTCAGATTAAAATATTAAATGGG | 5839 |
| hs132678503 | TTAAGTTCAGATTCAAGAAAAGG | 5840 |
| hs132680905 | GCTGAATATTGCGACAATATAGG | 5841 |
| hs132679203 | ATAATTTCATAGTAATAAGTAGG | 5842 |
| hs132677171 | ACTGCTGTTTATTCAAAAAGAGG | 5843 |
| hs132680748 | GTTTGATGTAGAGAAATAAGAGG | 5844 |
| hs132679107 | TGTCAGCTCCTAGATCTTCCAGG | 5845 |
| hs132681242 | GCTGCTTTTGAGCTATTTGAAGG | 5846 |
| hs132677538 | TTACTGCTGAAATTGAATATGGG | 5847 |
| hs132677036 | TATAGCTTTAGTTTTCAAAAGGG | 5848 |
| hs132679002 | ATTCAGGCCTGAGATACATTTGG | 5849 |
| hs132678403 | TTTACTTCTTCCATTCAATTGGG | 5850 |
| hs132678707 | TTTTGTTTTGATTATTCGGTTGG | 5851 |
| hs132681859 | GATAAAGGCAGAAATAGAATTGG | 5852 |
| hs132677534 | GCAGTTGCTCCTGGACAGTATGG | 5853 |
| hs132678427 | AACCTTCTCTGTTGTGGTGGCGG | 5854 |
| hs132680783 | TGATCCCCACTGAAAAAAACTGG | 5855 |
| hs132678768 | CACCCAGTGCATGTTTTCATAGG | 5856 |
| hs132682063 | GATATAATAAAAACTATTGCAGG | 5857 |
| hs132681436 | CCTCCTCCACTCCAAATCTTAGG | 5858 |
| hs132682142 | GATGTTTTTGAAATAGGTTAAGG | 5859 |
| hs132677988 | CTTCTGGCTGTGAGAGGCATAGG | 5860 |
| hs132677097 | GCTGGACTCTAAGTTCTATAGGG | 5861 |
| hs132683229 | AGAATATCGTCTATTCTTTGTGG | 5862 |
| hs132680354 | GGTAATATTAGACGAGCAAAAGG | 5863 |
| hs132680307 | AGTCATCATCAAAAAACAATAGG | 5864 |
| hs132681250 | TAGTTCTTGCTATCATTAAGAGG | 5865 |
| hs132679846 | CTTTGGGGACCCCTGCTTTGGGG | 5866 |
| hs132681309 | GTATTGTATGAAAGCTTCATTGG | 5867 |
| hs132680125 | TTTTGCCTGCTTCTGTAAAGTGG | 5868 |
| hs132683277 | GACATATTCTAGCTACTTGATGG | 5869 |
| hs132678952 | CAAAATCCAGATAAGCCTTTGGG | 5870 |
| hs132677307 | TTTGGTGTTTCCAATGTCTAGGG | 5871 |
| hs132678009 | AGTCATGTGCCATATGTAAATGG | 5872 |
| hs132681605 | AAGTTATTTTTACTCCATAAGGG | 5873 |
| hs132680438 | ACAGAGATATGCTCAGTGTTAGG | 5874 |
| hs132680169 | CTATGGGGTAAAAATGCAATAGG | 5875 |
| hs132680485 | AATGATTTTGGGGCTCATAATGG | 5876 |
| hs132682941 | AGTAAACATATATCTCTGATAGG | 5877 |
| hs132681745 | TTAGAATTTGTTTTTACTGTGGG | 5878 |
| hs132682806 | GGTTTACTGACTTAGATGATAGG | 5879 |
| hs132680295 | GTCTTTATCTCAATGCCATTAGG | 5880 |
| hs132681065 | AGGACCTATGAGAGATACTTTGG | 5881 |
| hs132680241 | ACTTCTGGCTCCCTTCCCTCAGG | 5882 |
| hs132680437 | TGATAACCCCTTTAGCAAACTGG | 5883 |
| hs132679197 | GGTCATATGGTCCCAATATAGGG | 5884 |
| hs132680761 | AATTCCATATGCCTAGTCTAAGG | 5885 |
| hs132680228 | ACATGGTTTATGAAAGAAATGGG | 5886 |
| hs132681632 | AGATGTAGACGTTATCTATAGGG | 5887 |
| hs132682071 | TGCTTCCAGTTTAAAAAAAACGG | 5888 |
| hs132682693 | ATAAGCTAGACTATTAAGATTGG | 5889 |
| hs132678716 | GATAAATTCAGCTTATGTTAAGG | 5890 |
| hs132679219 | GAGGGACTGTACTAATTTGTAGG | 5891 |
| hs132681264 | AAAAGCATAGGACTACAAAATGG | 5892 |
| hs132681576 | ACCATCGCCATCATTGACTATGG | 5893 |
| hs132678309 | GCTAATCTGTTCAGAGTCATAGG | 5894 |
| hs132678251 | AGTTCCCACTAGGAAATAGTAGG | 5895 |
| hs132681502 | GGCAGTGCCTATTCACTAACTGG | 5896 |
| hs132678332 | TCATTTCTCTTCAAAGCCTTAGG | 5897 |
| hs132679755 | GTCCTCAATCAAACACTAGATGG | 5898 |
| hs132682680 | CACAGACCAGAAACAGAAATTGG | 5899 |
| hs132680416 | GTATATAAGGGAATCATTACAGG | 5900 |
| hs132677301 | CTGAAACGTATTGATCCCTTTGG | 5901 |
| hs132680741 | TTACATGGCATCCAGAGAACAGG | 5902 |
| hs132682082 | ACACATTACCAATTTTAAATGGG | 5903 |
| hs132680769 | TCATAATATTTAGTTGATAAGGG | 5904 |
| hs132679639 | GGTTGGTGATCTTTGTTGTATGG | 5905 |
| hs132682889 | GAAAGACTCTGTAGGTCAATTGG | 5906 |
| hs132678598 | CTAACAACATTAGAATCATTAGG | 5907 |
| hs132681816 | TGTCAATGTTTCCATACCTTGGG | 5908 |
| hs132682570 | TTTTTATATTTGTTGTTAGTGGG | 5909 |
| hs132677122 | TTTTAATCGTGGAAGTTTAAGGG | 5910 |
| hs132679724 | GTAAATAAAATATAACAAATCGG | 5911 |
| hs132677634 | GAAGACACTGAAGAAAGAATAGG | 5912 |
| hs132682867 | GCAGAGTATGGTTTTTAAAAGGG | 5913 |
| hs132680807 | TTTTAATCTGAAGAAATCCTTGG | 5914 |
| hs132682226 | TTTATGTCATTGGTTTTGCCTGG | 5915 |
| hs132679727 | GGAACCTAGGAAATAGGAATTGG | 5916 |
| hs132679596 | TTTCTACTCTAAATTGAGTCAGG | 5917 |
| hs132681299 | GATCTGGCTTAATAATATATAGG | 5918 |
| hs132680472 | ATAGATTTCTGGAGTAAAACAGG | 5919 |
| hs132683221 | ATAACCATCTTATAAATATTCGG | 5920 |
| hs132677379 | AATAGAGTAACATACATATTGGG | 5921 |
| hs132679988 | TAATGTAGCGAAGGGTACTATGG | 5922 |
| hs132683216 | GAAGATCTATTTCAACTTGCGGG | 5923 |
| hs132681930 | GCTGTTGTAAAGGAGATTACAGG | 5924 |
| hs132680572 | TCTTAACTAATCACATTGCTTGG | 5925 |
| hs132679637 | CTATTCAGAAGAAGAAAAAATGG | 5926 |
| hs132680420 | TTAGCATATTAACTCCTTTGAGG | 5927 |
| hs132679113 | GCTTTACATTATAAATTGTATGG | 5928 |
| hs132678710 | AATAAGTAGGCGAAACCATTTGG | 5929 |
| hs132677346 | TCCAGGTGATAACATTTAACAGG | 5930 |
| hs132682391 | TATTCTGTTCCTTAGCCTAAAGG | 5931 |
| hs132678030 | TTGTTTTGCTTTACTTTGGTAGG | 5932 |
| hs132680024 | GAACATTTATATCCCTAATAGGG | 5933 |
| hs132681214 | TCAATAGGATTTTCTCAGAATGG | 5934 |
| hs132679038 | CCAAAAGATGAAAATATGTTTGG | 5935 |
| hs132682904 | GCAAGTACTTGATTTAAATTGGG | 5936 |
| hs132678525 | TCTCTTTCTGCGAGGACCTATGG | 5937 |
| hs132681100 | TGACAGATATTGTGATTTAAGGG | 5938 |
| hs132681391 | AGAGAAGAGAGGAGTCCTATAGG | 5939 |
| hs132679303 | AGTCTGTCTTTAGCACTAACCGG | 5940 |
| hs132679831 | TTGAAAGGAATTCTGCAGATAGG | 5941 |
| hs132677651 | TCTTAAGTATGGGGAGGGCGGGG | 5942 |
| hs132681751 | GCTTACTGTCTACTTTTTCAGGG | 5943 |
| hs132678133 | AACTGTATGAAAGAGCCTATTGG | 5944 |
| hs132678439 | TGGAGTCTTGTTGCATTCCTGGG | 5945 |
| hs132683190 | TTAGACACTTTTGAAGAAGATGG | 5946 |
| hs132677314 | GACCAGCTCCCAGAAAAATAAGG | 5947 |
| hs132682707 | AGTAGAATAGAAAGATATATAGG | 5948 |
| hs132678559 | CACTGTTTATGATGGATTAAGGG | 5949 |
| hs132677733 | ATTCATTTTAAAACTCTTAGTGG | 5950 |
| hs132679101 | GGCTTCTTCTTTGGGCTGTAGGG | 5951 |
| hs132680370 | TCACAAAGTTTCCCAGAAACAGG | 5952 |
| hs132682735 | GTGTGCCCATTCTTCCTAATAGG | 5953 |
| hs132679828 | AGAGGGTGCAGAGAGCTAAAAGG | 5954 |
| hs132678183 | CCTCCTGCACACCAAAGTTTAGG | 5955 |
| hs132681481 | GCAGGGACAATGATGCAAATTGG | 5956 |
| hs132678905 | CTGCAAGACAAGCAAAATATAGG | 5957 |
| hs132677040 | CTTTTTTCATCCGTCATCTTCGG | 5958 |
| hs132680139 | AATGAGATTTTGTAACCATAAGG | 5959 |
| hs132678648 | CTGCTTTTCAGGAAGTAACTTGG | 5960 |
| hs132680577 | AGAATAGGATGGTTTGTAAAGGG | 5961 |
| hs132678087 | TTTTTTTTTAATCAGCCAATAGG | 5962 |
| hs132677600 | GCACCAATCTTTTAAAAAGTTGG | 5963 |
| hs132679916 | GTTCTTTCTTTGGGGATATGAGG | 5964 |
| hs132680711 | TTTCTCCTAAAACATCTTCCTGG | 5965 |
| hs132681381 | GGAGAGTGAGAACGAGGATGAGG | 5966 |
| hs132682322 | CTTCCTGAGCTTGAAATGAATGG | 5967 |
| hs132678855 | GCTGAGGTTGAGAGCTTTCAAGG | 5968 |
| hs132678931 | AGAAGTTCAAAACAAGGCTTTGG | 5969 |
| hs132678223 | AGGAAAATTCCAAAGTTAAAGGG | 5970 |
| hs132682213 | GAATAATCTTTCCAAATAAAGGG | 5971 |
| hs132678868 | CCTGATGCTTTTGCAATAAAGGG | 5972 |
| hs132681659 | AGCCACATATAGTTGACATTTGG | 5973 |
| hs132678085 | TACTGGTTCCTGAACTCAATTGG | 5974 |
| hs132681197 | CAGCTACAAGACAACCTTGTAGG | 5975 |
| hs132679746 | GCACTGACTCATGGTTTAAAGGG | 5976 |
| hs132677302 | ATGACCTTCTGATTACAAAAAGG | 5977 |
| hs132677583 | TAGGTACCTGACATTTCAATAGG | 5978 |
| hs132680078 | CTTTTAATCCAGATACTTAGTGG | 5979 |
| hs132677935 | GATGCAGAATCAGTCTTAAGAGG | 5980 |
| hs132682189 | TTAAAACAATGCCATTATAGTGG | 5981 |
| hs132678574 | TTCTTACCAAAGAAACAAAAAGG | 5982 |
| hs132678378 | CTGCCCACCAAGAAGCTATAAGG | 5983 |
| hs132680624 | TGATAGCTTTGATTTTCAATAGG | 5984 |
| hs132680304 | TGTAATGTCCTTAACTCTTAGGG | 5985 |
| hs132682051 | ATGCTTTGTGTGTTGTTGCTAGG | 5986 |
| hs132683133 | GAATTAATCTTATCTAGAAATGG | 5987 |
| hs132677459 | TACTTCTGTGTACTTTTTGTGGG | 5988 |
| hs132681571 | GTTTAAAACTGTAGTATAAAGGG | 5989 |
| hs132678916 | TTTATAGCCCTTTCTTTTGAGGG | 5990 |
| hs132678406 | TATTAATATTCCCAATTGAATGG | 5991 |
| hs132677243 | CTGTGGGAGTGCAAGTCAATAGG | 5992 |
| hs132679803 | AGGAGGCTAGCTAACATAATAGG | 5993 |
| hs132683048 | ATTGGCATACAGAGTGTAATAGG | 5994 |
| hs132683084 | GGTTGTCTCAGCACAACATTTGG | 5995 |
| hs132678037 | AGTGGTATGGTACCATAAAAAGG | 5996 |
| hs132678134 | ATAAATACTTAGTGTCCAATAGG | 5997 |
| hs132682762 | TATTAATTATTTGAATGCTTTGG | 5998 |
| hs132678379 | GGATATTAACATTTTTTAAAGGG | 5999 |
| hs132682763 | TGAATGAGAAAGGTATCATTTGG | 6000 |
| hs132678186 | CTCAATGTCTTCCTAAACTTTGG | 6001 |
| hs132679485 | ACTAAGGAAAACAGGCTATAGGG | 6002 |
| hs132682662 | TATAGTTTATAGGCTAATAATGG | 6003 |
| hs132683051 | CTACCTTCTTGGCATCAAACAGG | 6004 |
| hs132680066 | TTTGTTTCCTCTACTGCAAATGG | 6005 |
| hs132680515 | ATTCATTTCCCAAGGCTCATTGG | 6006 |
| hs132679871 | AATCATCCATGTAGGACAATAGG | 6007 |
| hs132683250 | GAGACATCAAATCTAGATAATGG | 6008 |
| hs132679753 | AAAGCAGTACATATGCAAAAAGG | 6009 |
| hs132679722 | TAAAAAATAAAAGAGCTAAAAGG | 6010 |
| hs132679199 | TTTCTTTGTGGAAGGTCATATGG | 6011 |
| hs132678966 | TAGTATTCTGCCAAAATAAAGGG | 6012 |
| hs132678031 | ATAATCTTTTCCAGTTCAACAGG | 6013 |
| hs132679271 | TATGAGGTAGAGAGGGATTTGGG | 6014 |
| hs132679432 | GTATTGTTCAATCATTTTGCAGG | 6015 |
| hs132679726 | CATTCCAATTCCTATTTCCTAGG | 6016 |
| hs132680726 | GCTAAAACCCCTCTTGGCCTTGG | 6017 |
| hs132678405 | AATTGAATGGAAGAAGTAAAAGG | 6018 |
| hs132683011 | GTGGCTTGCTCACAGTAAATAGG | 6019 |
| hs132682658 | TCACAGCTAGTAATGTAAAATGG | 6020 |
| hs132678881 | TTACACCCCTAGGGTATAATAGG | 6021 |
| hs132677703 | TAGCAACCTTTAAATTTAAAGGG | 6022 |
| hs132679705 | ATAGTCTTCTCAATTCTATCTGG | 6023 |
| hs132678955 | GTTAAGAAGCCTTAGTTCAATGG | 6024 |
| hs132681568 | CATTTTATAGCTTTATAGATAGG | 6025 |
| hs132678805 | GAATTACCCAAAAATATAAAAGG | 6026 |
| hs132682131 | ATTGCTATAGTTAAGAGTTTGGG | 6027 |
| hs132680734 | GCCTCCTCTTTGAATAAATCAGG | 6028 |
| hs132680424 | ATCAGAGTCCCGGTAGAAACTGG | 6029 |
| hs132681269 | GTTGTCTTGGATACATTAAATGG | 6030 |
| hs132682020 | TTTGTCTCTAAATAAGGTTTAGG | 6031 |
| hs132678339 | TGTTTTATAATTCTCCAATAGGG | 6032 |
| hs132683122 | TGTTTGAGAAAAAATTTTATGGG | 6033 |
| hs132682337 | CATGGAAGACTCAAGTTATACGG | 6034 |
| hs132679643 | TCATTTCCAGAAAGACAATATGG | 6035 |
| hs132677504 | TATAGTATCTGGAAGCAAATAGG | 6036 |
| hs132681323 | GGGCTCCTGACGCTTCTTGCAGG | 6037 |
| hs132680035 | TAACTGGTACGTCTCCTTATAGG | 6038 |
| hs132682049 | CAGTTTATCTTTCCAGTAAGAGG | 6039 |
| hs132677472 | ATAAAATTGCTCCTAATTGTAGG | 6040 |
| hs132678494 | GGGGCATCTCAGTCCTAAATTGG | 6041 |
| hs132683160 | TTTTAGAGCTTTCTTCATAAAGG | 6042 |
| hs132681592 | CTTTTTTCCTATTATTTACTGGG | 6043 |
| hs132677198 | GAGAGGGAGTAAAGGATTATAGG | 6044 |
| hs132681760 | CAGTCTCAGGACAAGTAGATAGG | 6045 |
| hs132680739 | TTACTCATCACCCTGTTCTCTGG | 6046 |
| hs132679245 | TCATTGAATTAATCACTAATAGG | 6047 |
| hs132681429 | TTGCAGCTCCTCATATTTGATGG | 6048 |
| hs132678274 | TCATTTCCTGCCTACCTGATTGG | 6049 |
| hs132682062 | TTTTTATTATATCTGCACTATGG | 6050 |
| hs132682342 | TCCATGAGAACCAAAACTATTGG | 6051 |
| hs132681185 | GGACTCTTCTCATCCAATTCCGG | 6052 |
| hs132679442 | GGAGCACAGCCTTTTGAAACTGG | 6053 |
| hs132678512 | ATTGTTGTCTGATTTATTACCGG | 6054 |
| hs132682388 | TGGCCCTACAGCTAAGAAAATGG | 6055 |
| hs132680242 | ATATCTCTAGTCTCTACTTCTGG | 6056 |
| hs132683188 | TGAAGAAGATGGGAATTTAGAGG | 6057 |
| hs132679332 | TCTGAAATCATATTTCGATTTGG | 6058 |
| hs132677075 | ATCTTACATGAGATACTTAGAGG | 6059 |
| hs132681967 | CTTGTACTATGAAGGATACTTGG | 6060 |
| hs132679335 | GTTTTGTTTTTATTCCAAATAGG | 6061 |
| hs132683082 | GCTGAGACAACCTACTTAAATGG | 6062 |
| hs132682163 | TTAATTGTTGAAACCTATAAAGG | 6063 |
| hs132679574 | GTATTATTCTTCCACATAATGGG | 6064 |
| hs132680392 | GCAGTGGCAGACTAGCTTTTGGG | 6065 |
| hs132682412 | TTGTCTCCTGAAATAATAATAGG | 6066 |
| hs132682925 | ATAGACATAGAGACCTAAAATGG | 6067 |
| hs132683245 | TCTTAAAGCCTTGGGTTTAAGGG | 6068 |
| hs132679553 | TCCCATGTCCCCCCAACTTTGGG | 6069 |
| hs132682963 | TCTTGACAATTCCTTTTAGAAGG | 6070 |
| hs132677728 | TTCAAGGTATGATCTTAAAAAGG | 6071 |
| hs132679854 | TTACTTTTGTAATTTAATAAGGG | 6072 |
| hs132682030 | ATAGCTCTTAGTAAATAGTATGG | 6073 |
| hs132682985 | CCAAAATGCAACCATTGTTTAGG | 6074 |
| hs132683212 | GCTTTGATTTCTATATTAACAGG | 6075 |
| hs132682023 | CTAGTGAGATGCTCATTAACTGG | 6076 |
| hs132678889 | CCTATAGATTAGAAAAAAATCGG | 6077 |
| hs132677252 | CCAGTTTTAGTAATTGTATAGGG | 6078 |
| hs132678080 | GGTCATGTCAAACTACTTACTGG | 6079 |
| hs132679221 | AATCAATCGGCCAAGCAAAATGG | 6080 |
| hs132680598 | TGTGTTCTCTAACTATGATTAGG | 6081 |
| hs132680073 | TTTGATATCCTCAGTAACTATGG | 6082 |
| hs132677096 | GCAAAGAAAATATTCCTAAAAGG | 6083 |
| hs132678248 | TCAAGACTAGTCTTAGTATAGGG | 6084 |
| hs132680299 | CCAAGCCGCAGAAATAAATTAGG | 6085 |
| hs132680965 | GGATTATATGAATAATGCTTTGG | 6086 |
| hs132680042 | TCTCAGACATTACAAACTTTAGG | 6087 |
| hs132680097 | GCCTGACTATCGTAAATTTAGGG | 6088 |
| hs132681394 | TATTTCCCAAATTATCCTATAGG | 6089 |
| hs132678965 | ATAGTATTCTGCCAAAATAAAGG | 6090 |
| hs132678957 | GGGAAGACTAATTAAAAAAATGG | 6091 |
| hs132681593 | GAAGAAACCCAGTAAATAATAGG | 6092 |
| hs132680333 | TTAGTATTACCGGTTGTCTAAGG | 6093 |
| hs132680816 | AATGTATTCTTAACAAGCTTTGG | 6094 |
| hs132683131 | GTATACACGAGGCTTTTTAAAGG | 6095 |
| hs132679869 | CTTTATCTATGCTTTGCTATAGG | 6096 |
| hs132681674 | TCTGTGCCTTGATTAAAAAAAGG | 6097 |
| hs132681888 | TTTCTGTTGCCAATTTATAATGG | 6098 |
| hs132683146 | GCTACGGAAGGGATATTTAAAGG | 6099 |
| hs132683234 | GATCTCCTAGTGGATTGTTTTGG | 6100 |
| hs132681807 | TTGCCCATCGTTATGTTTAGTGG | 6101 |
| hs132682450 | TTTTTGGTACGCTTTCAAACTGG | 6102 |
| hs132682906 | ATTGATTCTCAACTGGAAAAAGG | 6103 |
| hs132677039 | CGAAGATGACGGATGAAAAAAGG | 6104 |
| hs132682203 | TCTTATTTCAGTGAACTAAAAGG | 6105 |
| hs132681456 | AGTTTCCCCTTGCTGCTTCATGG | 6106 |
| hs132677453 | CATAAAGTTGGACCACTAAAAGG | 6107 |
| hs132677435 | GGTGGAGGATGGGAATAATTAGG | 6108 |
| hs132679355 | AATCATATGTAGTCATTCTATGG | 6109 |
| hs132678219 | ATATTTTTTCCCTTTAACTTTGG | 6110 |
| hs132678014 | GCAGAGGTTGTCTAACCTTTTGG | 6111 |
| hs132681475 | GATAGAGGTGAGAAAGGTTCTGG | 6112 |
| hs132681329 | GAATTATTAGACACAAAAATTGG | 6113 |
| hs132679845 | GACCTACCCCCTAAGCTTTAGGG | 6114 |
| hs132682839 | GGTTTAGCTCGCTAGCTAACAGG | 6115 |
| hs132678279 | CTAATTGTTGTTAAATTGATTGG | 6116 |
| hs132682627 | GTGCAAACAGTTATATTTAATGG | 6117 |
| hs132682025 | CTAGGGGATTAATATTAATAAGG | 6118 |
| hs132681517 | AATTAGATGATGAGACAAAATGG | 6119 |
| hs132677463 | GCTTGACTATCATCACATTCAGG | 6120 |
| hs132677133 | TGAAGTACCCGGGCATTTACAGG | 6121 |
| hs132680145 | TTACAAAATACAATTCCTTATGG | 6122 |
| hs132681972 | TTCCATTTAATGCTTACTAATGG | 6123 |
| hs132681122 | ACCTTACTATATACATATAATGG | 6124 |
| hs132682310 | CCTTCCAGAAATCACTAAAATGG | 6125 |
| hs132683035 | GGTTTTACATAGATCTAAAATGG | 6126 |
| hs132679030 | TCTATAGCAAATGAAAAAAAAGG | 6127 |
| hs132678091 | TGTTGGAGCTTTCAGCCTATTGG | 6128 |
| hs132677645 | TTAAGCCTGTTTACCATTGCAGG | 6129 |
| hs132681613 | ATTCTCATATAACATGTTTTCGG | 6130 |
| hs132678155 | AAAACTTGTCACTATCAAAATGG | 6131 |
| hs132679611 | ATATCTATTGAAGATATAATTGG | 6132 |
| hs132679293 | TGAAAAGTAGGCCAGAAATTTGG | 6133 |
| hs132679084 | GTGACCACTCGAACATTTACAGG | 6134 |
| hs132678359 | AATAAATTCATCTAATAAAAAGG | 6135 |
| hs132679024 | TTGTATATAAAAAGTTAAATAGG | 6136 |
| hs132682726 | CTACAATGTGACTTTCTAAATGG | 6137 |
| hs132681580 | CTCATTCTCCTACTACTCTTTGG | 6138 |
| hs132678179 | TCCATACTATACACATAATATGG | 6139 |
| hs132683173 | GTTTTTGGTTAATTGATAAATGG | 6140 |
| hs132677381 | TGTGCATTCGAACTTTAAATAGG | 6141 |
| hs132679191 | GCAGTAGTTGACATTAAAAATGG | 6142 |
| hs132682544 | CAGAATAGTTGGGAACTAAATGG | 6143 |
| hs132677612 | ACTTAAAGTTACATCAAAAATGG | 6144 |
| hs132677212 | TGAATATGACTAATAGAAATTGG | 6145 |
| hs132681658 | GGAAGAATTTGACAGAAATATGG | 6146 |
| hs132678208 | TGATTTCCTTGGGTTCCTTTTGG | 6147 |
| hs132681604 | GTTTTTTTCCCTGAAATTTTCGG | 6148 |
| hs132682383 | CTAATAAACTAAAGTCAAATTGG | 6149 |
| hs132679409 | CTTAGAGCTCTTTCAATTATGGG | 6150 |
| hs132681418 | ATTTAGATCTAATTTGTATAGGG | 6151 |
| hs132682951 | CTAGCAACTATCATGCTAATTGG | 6152 |
| hs132677793 | TATAATTGCCATCAAATAAAAGG | 6153 |
| hs132677773 | TTTAGGGGTCAGCTTTTAAAAGG | 6154 |
| hs132679778 | CTAAAAGGCATTTTTAAATATGG | 6155 |
| hs132677602 | TGAACCTTAAATTTAAATAATGG | 6156 |
| hs132683284 | AAGTTGTGAAATTTTCAAAATGG | 6157 |
| hs132679843 | GCTTTTATGTGTTTACTTAGAGG | 6158 |
| hs132677958 | TCTGAAAGTTAGCCCTTTAGAGG | 6159 |
| hs132681023 | GCGTTTTCTACTCTTAATAAAGG | 6160 |
| hs132680912 | CTGTATTCCATCAGAAAAAATGG | 6161 |
| hs132679886 | CTATTTGCTCAACATTATTCAGG | 6162 |
| hs132679851 | TTATTTTTCTGTCAGTAAAATGG | 6163 |
| hs132682054 | AGACACTTGAATATGAAAATTGG | 6164 |
| hs132680050 | CATACCTTCCTTTACGTTATGGG | 6165 |
| hs132680875 | TTTCTGGAATGACTGATCTTAGG | 6166 |
| hs132679004 | GCTTGCACAGAATTTTTAATTGG | 6167 |
| hs132680616 | GGTGATTGCATTGATATTAAAGG | 6168 |
| hs132677795 | ATTCCTTTCCTTTTATTTGATGG | 6169 |
| hs132678600 | GGACTGTTGTCATAAGTTATAGG | 6170 |
| hs132683003 | ATTTTTGTCCCAACAATAATAGG | 6171 |
| hs132677402 | TCAGAATGTTCAATTTTATAGGG | 6172 |
| hs132677265 | CTTGAAAAGGAAGCCATTAAAGG | 6173 |
| hs132680106 | GCATAACGTTTACATCAATATGG | 6174 |
| hs132679817 | TCTCCTTCATTGTAAATTTAGGG | 6175 |
| hs132678260 | ACCACTAAAGAAAATCTTTTCGG | 6176 |
| hs132683138 | GATAGGGCTTATTTTTAAAATGG | 6177 |
| hs132682079 | TCATAACACCCATTTAAAATTGG | 6178 |
| hs132680404 | ATTATGGCACAAAAACTATTTGG | 6179 |
| hs132677532 | TATTTTTGCATGCAAATAATTGG | 6180 |
| hs132679100 | GTTCAATATAACTCATTAAATGG | 6181 |
| hs132681534 | CTTATGCTGGTCAGATTAAATGG | 6182 |
| hs132683261 | TTACCTTGAAAACTAGTATTAGG | 6183 |
| hs132681887 | TTTTAATCTCCATTATAAATTGG | 6184 |
| hs132679717 | CTATTCAACTATAAAGTAAATGG | 6185 |
| hs132677155 | ATATCTAACCTGGAATAATTTGG | 6186 |
| hs132681720 | CCAAAAGTAAACTTAAAATTAGG | 6187 |
| hs132683203 | GTTAGAGGATGCAAATATTTTGG | 6188 |
| hs132678742 | CTTTCTCATTCCTACTTAATAGG | 6189 |
| hs132682053 | ATAAAATCAGTTTTTATTAAAGG | 6190 |
| hs132680340 | GATCCTTCCTTAGCTGTTAATGG | 6191 |
| hs132682465 | GTAATCAGACAACCTTAATTTGG | 6192 |
| hs132682665 | ATAACATGTTGTGTAATTATTGG | 6193 |
| hs132679340 | GATATTGGCCCTTTCTAAAAAGG | 6194 |
| hs132682811 | TGAAACATCGAATACAATTTTGG | 6195 |
| hs132682164 | TATAGGTTTCAACAATTAATTGG | 6196 |
| hs132681050 | TCTTTTTGTGGTAAGAAATATGG | 6197 |
| hs132678527 | CCTTAGACCTAGATTCTTAAAGG | 6198 |
| hs132678791 | AATCATTACCTCCAACTTAATGG | 6199 |
| hs132677774 | ATATGTGCTTTTGAGATAATTGG | 6200 |
| hs132677513 | TCATACTGTCCTAAGAAAAATGG | 6201 |
| hs132682306 | ATAGCAACCAAAATTCTTTCTGG | 6202 |
| hs132677010 | TGCCAAATGAAAATCATACGAGG | 6203 |
| hs132676914 | TCAGTCAGGGAAATGACCAGAGG | 6204 |
| hs132676992 | GATCAAGGTCTTACACTGGGGGG | 6205 |
| hs132676974 | TTAGCGTTAAGTGTTACAGGAGG | 6206 |
| hs132676981 | AGGGCAAAGATAAGAGACGAAGG | 6207 |
| hs132676896 | AATCACCAAACATTTACACATGG | 6208 |
| hs132676746 | GTAAACGAACAGAGCCTGTGAGG | 6209 |
| hs132676985 | TTATTTATGACTATAGACTGGGG | 6210 |
| hs132676819 | GGAACTACTTGAGAAAGACCAGG | 6211 |
| hs132676877 | TATCTTCTACTCCTAATGGGAGG | 6212 |
| hs132676913 | TAGATGGATTGACTCAGTCAGGG | 6213 |
| hs132677014 | TGACATCTAGTAGAATGACTAGG | 6214 |
| hs132676918 | AGACAACAGCCTCCTAACAGAGG | 6215 |
| hs132676901 | GCCAGTATAAGGTAAGCTAGTGG | 6216 |
| hs132676891 | TCTGTGATTAGGACAAACAATGG | 6217 |
| hs132676760 | TCATGCAGTAATCCCCAACTGGG | 6218 |
| hs132676860 | CCCCTATTAGAGTCTCCTTGGGG | 6219 |
| hs132676803 | TTAGGTGAGAACTTTGCAAGTGG | 6220 |
| hs132676868 | GTACCTTCCTTTTAACCCTGGGG | 6221 |
| hs132676861 | CCCCAAGGAGACTCTAATAGGGG | 6222 |
| hs132676919 | GACAACAGCCTCCTAACAGAGGG | 6223 |
| hs132676986 | TATTTATGACTATAGACTGGGGG | 6224 |
| hs132676970 | AATATATACCGAGAAAAGAGTGG | 6225 |
| hs132676940 | AATAATAACTTTAGTCAAGGGGG | 6226 |
| hs132676889 | AAGGCAAAATCCTCAGACCAGGG | 6227 |
| hs132676899 | TATAAGGTAAGCTAGTGGTAGGG | 6228 |
| hs132677009 | ATTTTTACTTTTTCTGCAGGCGG | 6229 |
| hs132676838 | TTCTTGGAGGAAAATCTAAGAGG | 6230 |
| hs132676750 | GCTAAAAACGTACCAGGCCAAGG | 6231 |
| hs132676996 | TGTTGATCAAGGTCTTACACTGG | 6232 |
| hs132676856 | GCAGTTTCTGATCCTGAAGTGGG | 6233 |
| hs132676967 | AGGTTTCTGGCAGACAGTTGGGG | 6234 |
| hs132676869 | CATCCCCAGGGTTAAAAGGAAGG | 6235 |
| hs132676958 | TGGGAGGGAAAAATTAGCAAAGG | 6236 |
| hs132676865 | AAAATAAGAAATACTCCCCAAGG | 6237 |
| hs132676833 | TTAATGTTTTGTGAAATCAAAGG | 6238 |
| hs132676991 | ATCAAGGTCTTACACTGGGGGGG | 6239 |
| hs132676989 | AAGACGATTATTTCAGTCAGGGG | 6240 |
| hs132676928 | ACGAACATTTGGAAGACACTGGG | 6241 |
| hs132676821 | GCAAATGATAAGGAGTTCTAGGG | 6242 |
| hs132676956 | GGCCTTCTGTGGAATTTGGGAGG | 6243 |
| hs132676752 | GTTTATGCTAAAAACGTACCAGG | 6244 |
| hs132676922 | TGTCCTGGCCCTCTGTTAGGAGG | 6245 |
| hs132676990 | TCAAGGTCTTACACTGGGGGGGG | 6246 |
| hs132676820 | TGATAAGGAGTTCTAGGGTGAGG | 6247 |
| hs132676754 | AAGTGTTGCCATTCTTAGAGTGG | 6248 |
| hs132676802 | ACTTTGCAAGTGGTTTGCATTGG | 6249 |
| hs132676790 | AATGGTGAGTGAGTGGAGAAGGG | 6250 |
| hs132676938 | TTATAGCTGAACTGTTTGCTGGG | 6251 |
| hs132676900 | GTATAAGGTAAGCTAGTGGTAGG | 6252 |
| hs132676816 | ATACTTTCCAGCAAAGCTCCTGG | 6253 |
| hs132676930 | TCTGTATTCGGTAACAAAGTAGG | 6254 |
| hs132676828 | AGTTACCTTGGAGAGTAAGATGG | 6255 |
| hs132676849 | GTGGCTTTGGTCTAACTACTAGG | 6256 |
| hs132676945 | ATGTGAGATTAATACTAAAACGG | 6257 |
| hs132676846 | CTATATATACACAAGAAGATTGG | 6258 |
| hs132676759 | TTCATTCTATAGCCCAGTTGGGG | 6259 |
| hs132676872 | TATTGGCTCAGGACATCCCCAGG | 6260 |
| hs132677000 | CCACATGTATAAACTCATACAGG | 6261 |
| hs132676773 | GCTCTAAACCTCAATCACTTTGG | 6262 |
| hs132676839 | CATCATTCTGACATTCTTGGAGG | 6263 |
| hs132676817 | GGAGCTTTGCTGGAAAGTATAGG | 6264 |
| hs132676851 | CATAATTGAAAGGAAATATGTGG | 6265 |
| hs132676853 | AATGTTTGAATATGATACAAAGG | 6266 |
| hs132676975 | CGTTAAGTGTTACAGGAGGCAGG | 6267 |
| hs132676948 | AGGACTTCAGAATTTAATTGAGG | 6268 |
| hs132676775 | TTAGGGAACCAAAGTGATTGAGG | 6269 |
| hs132676898 | CCTTATACTGGCCAATCTCATGG | 6270 |
| hs132676950 | CTTCAGAATTTAATTGAGGAGGG | 6271 |
| hs132676916 | GAAGTGATCACGCAGATACCTGG | 6272 |
| hs132676890 | GGGCTGACAGATCTGTGATTAGG | 6273 |
| hs132676942 | GAAATAATAACTTTAGTCAAGGG | 6274 |
| hs132676841 | TCCCACTTTCCACCAAAAAGAGG | 6275 |
| hs132676867 | AGTACCTTCCTTTTAACCCTGGG | 6276 |
| hs132676917 | TCTCCTGTGTTGCAATCCTCTGG | 6277 |
| hs132676744 | TTTCATTTCTAGTAGATTGTCGG | 6278 |
| hs132676784 | GCTAATAAGGGCTTGTAGTAAGG | 6279 |
| hs132676810 | GTAGTGCCCTGGACAAGGTCTGG | 6280 |
| hs132676785 | CTCTGGGCAATAGCTAATAAGGG | 6281 |
| hs132676876 | TAGTATACATGCCTCCCATTAGG | 6282 |
| hs132676886 | GGATCGGGAGCATTTGTTAGAGG | 6283 |
| hs132676932 | TGTAAATTGTTCACAGTCATAGG | 6284 |
| hs132676982 | ATGATTTAGCAGAATTTGATAGG | 6285 |
| hs132676879 | GATATATCTTCTACTCCTAATGG | 6286 |
| hs132676934 | TTTACATGATGACCTCAAATTGG | 6287 |
| hs132676863 | CTCCCCAAGGAGACTCTAATAGG | 6288 |
| hs132676870 | AGGACATCCCCAGGGTTAAAAGG | 6289 |
| hs132676921 | GCTGTTGTCTCTTCACTTCCAGG | 6290 |
| hs132676832 | TTTGATTTCACAAAACATTAAGG | 6291 |
| hs132676847 | TTGACAGTATACAAGTTTAATGG | 6292 |
| hs132676745 | CTTAATAGTGAAATTATAAATGG | 6293 |
| hs132676266 | AAGTTATATCTCATTCACAGGGG | 6294 |
| hs132676139 | AAAGCATTCAGATCACTAGGGGG | 6295 |
| hs132676427 | ATTTGATAGAGATTTAACTGTGG | 6296 |
| hs132676332 | TTGGAAATCCAGGTCCACAGTGG | 6297 |
| hs132676686 | ACAGGGGAAGAGTCACCAAGTGG | 6298 |
| hs132676685 | CCTCCAATGCCATGACACAGGGG | 6299 |
| hs132676568 | GCTTCATATTGCCTACCATGTGG | 6300 |
| hs132676471 | TGAGGAACAGGGTTAAACTGAGG | 6301 |
| hs132676650 | TGTAGCAGTGAAGAAAAGAGCGG | 6302 |
| hs132676220 | CATAGGACAAGAACTTAAGGGGG | 6303 |
| hs132676178 | ATGAAGACTCTAGAGGAGGGAGG | 6304 |
| hs132676403 | ATCAATTGTCTATCACAAGGTGG | 6305 |
| hs132676438 | TTACAAACAAAAGTGGGACAGGG | 6306 |
| hs132676209 | TATGTCAAAGCAAACCATGGAGG | 6307 |
| hs132676556 | ATATGTCATAAACTTGACAGTGG | 6308 |
| hs132676573 | TGTCAGAGATTACAGCCACATGG | 6309 |
| hs132676535 | TGTATAGTGAATGTATCACTTGG | 6310 |
| hs132676600 | TCTGAGCTAGAGCTTTAATGGGG | 6311 |
| hs132676477 | AGAGGGACAGGCTAAAATGGAGG | 6312 |
| hs132676173 | ATGTCTTCTTCAAACTACCAAGG | 6313 |
| hs132676460 | CTCTACAGATATAAGCTGAGTGG | 6314 |
| hs132676187 | CAGACCAGACCGTCAATGTGAGG | 6315 |
| hs132676129 | GTGTCTCACGGAAGCCCAAGGGG | 6316 |
| hs132676545 | TGGCCATTCCATAGGCTCAGGGG | 6317 |
| hs132676201 | ATTAAATAGTAGGGTCACTGTGG | 6318 |
| hs132676546 | CAGAGCTCAGTAGATCCACATGG | 6319 |
| hs132676097 | AGTGACAAGTCTTATGACAGTGG | 6320 |
| hs132676505 | TTATGAATGTGATCCAGACAAGG | 6321 |
| hs132676416 | AAATCATAAGAGGGAAGACGAGG | 6322 |
| hs132676265 | AAAGTTATATCTCATTCACAGGG | 6323 |
| hs132676229 | ACTCTACCTCCTAAATGACTGGG | 6324 |
| hs132676667 | ATAAGCTGAGCAACATTTGGGGG | 6325 |
| hs132676709 | TGAAGTAAAATCTGAAAACAGGG | 6326 |
| hs132676504 | TGAATGTGATCCAGACAAGGAGG | 6327 |
| hs132676198 | ACAACACTGATACACACAGTAGG | 6328 |
| hs132676175 | GATAACCATGAAGACTCTAGAGG | 6329 |
| hs132676527 | TTGTCTCCAGAAGAATAGGAAGG | 6330 |
| hs132676547 | TCTCCCCTGAGCCTATGGAATGG | 6331 |
| hs132676621 | AGATTTCTATATATGGCCCGTGG | 6332 |
| hs132676053 | TGGCACTCCTTGGAGCTAGGTGG | 6333 |
| hs132676318 | GTGTAAAAGCACATGCTGCAGGG | 6334 |
| hs132676551 | TCAAGTCTGAACTCTCAACAAGG | 6335 |
| hs132676525 | AGACACGCTGAGAGTCACTTCGG | 6336 |
| hs132676371 | AAAGCAAGAGGTATACTAAGGGG | 6337 |
| hs132676401 | GTAACTTCAAATCTACAATGTGG | 6338 |
| hs132676503 | GAATGTGATCCAGACAAGGAGGG | 6339 |
| hs132676651 | GATATTTCCAAAGAGTCCATCGG | 6340 |
| hs132676372 | AAGCAAGAGGTATACTAAGGGGG | 6341 |
| hs132676408 | ATAGTGTTGAAATATACCCTAGG | 6342 |
| hs132676326 | GATATATACAACCTAAACGGTGG | 6343 |
| hs132676495 | TGTGTGGACAAACTTATCACTGG | 6344 |
| hs132676126 | AGGTAGGCAACTGTGTCTCACGG | 6345 |
| hs132676415 | TAAGCAAGGAAATCATAAGAGGG | 6346 |
| hs132676593 | GGAACCTTTACCTTAGCCTGTGG | 6347 |
| hs132676492 | AAACCTATGAAAAGAAACCCTGG | 6348 |
| hs132676635 | GGGCTCCTCTAGCCCCAAAGGGG | 6349 |
| hs132676148 | GTATGCATAAGATATTGGCCAGG | 6350 |
| hs132676452 | GTGCAGTGATAACTTTATTGCGG | 6351 |
| hs132676335 | TGAAATACGGAGATAAGGAAGGG | 6352 |
| hs132676135 | GAATGAAGAAAAGTATCAGGTGG | 6353 |
| hs132676538 | GCCCTATGAAAGACACCATGTGG | 6354 |
| hs132676668 | GTTGCTCAGCTTATCTGGCAGGG | 6355 |
| hs132676443 | AGGTAAAGCAAATCCCAGCTAGG | 6356 |
| hs132676515 | GTATAAAACTACATTTGTGAAGG | 6357 |
| hs132676050 | AGTACTTCCACCTAGCTCCAAGG | 6358 |
| hs132676035 | AATCAACCTGTTAAAGAAAGGGG | 6359 |
| hs132676426 | AATACATCTTGGGGAGCGGGGGG | 6360 |
| hs132676672 | TCTTAAAATAACCATAAGCAGGG | 6361 |
| hs132676253 | AAATAAGACGTCAGAATGAGGGG | 6362 |
| hs132676197 | TGTGACAAGCACTCTATGGTTGG | 6363 |
| hs132676448 | TGGGAGACTATGCTACTAAGTGG | 6364 |
| hs132676695 | CAGTCACAGCTAAACCAAGTAGG | 6365 |
| hs132676327 | ATATATACAACCTAAACGGTGGG | 6366 |
| hs132676465 | ACAGGTTAAACGCCAGGACAGGG | 6367 |
| hs132676146 | AGGGACAGAATTGTATTCAGTGG | 6368 |
| hs132676154 | ATTGTTTCTAGGAAGGCAGGTGG | 6369 |
| hs132676080 | AAACATACTCACTCATACTGCGG | 6370 |
| hs132676232 | TCATAGCCCAGTCATTTAGGAGG | 6371 |
| hs132676693 | AAACCAAGTAGGCAGGTCAGAGG | 6372 |
| hs132676594 | TTCTGCATGAGAATGTCTTGAGG | 6373 |
| hs132676207 | TGGAGGATAGAAAACCTCCATGG | 6374 |
| hs132676113 | TGAACTTTTTGACACTGGGGGGG | 6375 |
| hs132676241 | CTTGAATAAAATATTGACGATGG | 6376 |
| hs132676128 | TGTGTCTCACGGAAGCCCAAGGG | 6377 |
| hs132676222 | ACTAGTCTGGCGATAGAAGAGGG | 6378 |
| hs132676344 | TATTCTTAACATTCTATGCGTGG | 6379 |
| hs132676210 | CATTATGTCAAAGCAAACCATGG | 6380 |
| hs132676058 | GTTAGGTCTCTAATAAACAATGG | 6381 |
| hs132676190 | AGAGAAGTTACTGTATGGCAGGG | 6382 |
| hs132676555 | TTGAGAGTACAAATAACTGTAGG | 6383 |
| hs132676512 | AAACCCATGAGAAATGGCTCAGG | 6384 |
| hs132676707 | ACAAAGTCCTGCAGTCAACTCGG | 6385 |
| hs132676238 | CCATGTTCCTGAAGGAAAGTGGG | 6386 |
| hs132676200 | TGGTTGGAAATTAAATAGTAGGG | 6387 |
| hs132676219 | GCATAGGACAAGAACTTAAGGGG | 6388 |
| hs132676073 | GATTCCTCATATGGCAGTAGAGG | 6389 |
| hs132676221 | ATGCCTTTAAAATAGACAGCAGG | 6390 |
| hs132676069 | CACTCCTCAAATGTTTAAGGTGG | 6391 |
| hs132676137 | AGAAAGCATTCAGATCACTAGGG | 6392 |
| hs132676625 | TTAAAACCTCCGAAACTCCACGG | 6393 |
| hs132676714 | TGTCTTTTCCCTTCCCTCTGTGG | 6394 |
| hs132676078 | ATCAATAGATAAATTCATAGTGG | 6395 |
| hs132676059 | TAACATTCATTCTAAAACAGAGG | 6396 |
| hs132676629 | ACTGATTATGTCTAGCCTTGTGG | 6397 |
| hs132676606 | ATAATGGCTTAAATTTGAGAAGG | 6398 |
| hs132676466 | TTAAACGCCAGGACAGGGAAAGG | 6399 |
| hs132676567 | GGGGTGAGAAGAAGAAAGCATGG | 6400 |
| hs132676678 | AAGTGCCTGGTGAAATGTATTGG | 6401 |
| hs132676094 | GTCTTATGACAGTGGATAAGGGG | 6402 |
| hs132676623 | TATGGCCCGTGGAGTTTCGGAGG | 6403 |
| hs132676076 | ATAGTGGCTTTTCCTTATGGAGG | 6404 |
| hs132676570 | CTGTAATCTCTGACACACAAAGG | 6405 |
| hs132676366 | CCTATTATGTTCACATATGGAGG | 6406 |
| hs132676597 | GGTTTTGTCCTCTAACGCATAGG | 6407 |
| hs132676624 | TAAAACCTCCGAAACTCCACGGG | 6408 |
| hs132676359 | GACTTGAGCTTAAAAAGTTGGGG | 6409 |
| hs132676430 | TTAGAACTTAGTTATATCCAAGG | 6410 |
| hs132676534 | AGTGAAGTAGGAGTGGAAAACGG | 6411 |
| hs132676674 | AGTTTATCTTTGAAAGAGAGAGG | 6412 |
| hs132676283 | GTATTTTACACTGCATATGAGGG | 6413 |
| hs132676181 | GGTATTTGTCATGCCTGAAAGGG | 6414 |
| hs132676691 | CTAATCTCCTCTGAACCACTTGG | 6415 |
| hs132676062 | CTAGACAGAGATAAGAGAGCTGG | 6416 |
| hs132676425 | TAATACATCTTGGGGAGCGGGGG | 6417 |
| hs132676162 | ATATAATCACATTTTGCAAGTGG | 6418 |
| hs132676530 | CGTGGTTTCTGGCTAATTTGGGG | 6419 |
| hs132676081 | AACATACTCACTCATACTGCGGG | 6420 |
| hs132676627 | CCTGTTGACAATCAGCGAAACGG | 6421 |
| hs132676500 | GTATGCAGTTTAAAAATGGAAGG | 6422 |
| hs132676288 | TTTTAAAATTAGCTAAGGGAGGG | 6423 |
| hs132676571 | TGTAATCTCTGACACACAAAGGG | 6424 |
| hs132676488 | TGAGTGATTTTTTTAAACCAGGG | 6425 |
| hs132676583 | ATAAATCAAAATGCTCAAGCAGG | 6426 |
| hs132676150 | CTGTTATTATAACAATTCACAGG | 6427 |
| hs132676374 | AGAGGTATACTAAGGGGGATGGG | 6428 |
| hs132676596 | TCTCATGCAGAAACCAAACTTGG | 6429 |
| hs132676590 | TCAGCCACAGGCTAAGGTAAAGG | 6430 |
| hs132676239 | TCCATGTTCCTGAAGGAAAGTGG | 6431 |
| hs132676510 | CAGCCTGAGCCATTTCTCATGGG | 6432 |
| hs132676719 | GCAAATGCATGCTGCCACAGAGG | 6433 |
| hs132676381 | TATTTTACAGGCCTATTGTGTGG | 6434 |
| hs132676716 | GCATGCTGCCACAGAGGGAAGGG | 6435 |
| hs132676424 | ATAATACATCTTGGGGAGCGGGG | 6436 |
| hs132676688 | AGAGTCACCAAGTGGTTCAGAGG | 6437 |
| hs132676677 | TTAGGGAGCTACTAAGTGCCTGG | 6438 |
| hs132676093 | AGACTTGTCACTGTGTAAGCAGG | 6439 |
| hs132676703 | AGACAGCGTTTAATAAGTGTTGG | 6440 |
| hs132676613 | TATATTTTCTGTAAGAAATGTGG | 6441 |
| hs132676684 | GCCTCCAATGCCATGACACAGGG | 6442 |
| hs132676250 | GAAAGAAGAATAGCAGACTAGGG | 6443 |
| hs132676611 | AACCTCCTAATGTTTTAGTAGGG | 6444 |
| hs132676046 | TAGATTACTGCAGATTAAAGAGG | 6445 |
| hs132676458 | CCAATTTGCATTCTTTGTGGTGG | 6446 |
| hs132676324 | TCAATACATGCATCTCTCAATGG | 6447 |
| hs132676484 | AATATTCCTTTAAAAATCAGAGG | 6448 |
| hs132676290 | TTAAAATTAGCTAAGGGAGGGGG | 6449 |
| hs132676192 | ATAAAAGAGAAGTTACTGTATGG | 6450 |
| hs132676553 | CTTGCACATCCAACTTCATAGGG | 6451 |
| hs132676608 | TTCCCTACTAAAACATTAGGAGG | 6452 |
| hs132676417 | GGAAGACGAGGACACAAGCTTGG | 6453 |
| hs132676491 | CATAGGTTTATAGAATTAGGAGG | 6454 |
| hs132676109 | AGCCCAGTACCTGAATATAGTGG | 6455 |
| hs132676368 | CTAATTTCCCTTAAAGCAAGAGG | 6456 |
| hs132676244 | CTTTTACTGTAAACCATCATGGG | 6457 |
| hs132676206 | TTATTCAAATGTAATTTTGGAGG | 6458 |
| hs132676251 | GTAAATAAGACGTCAGAATGAGG | 6459 |
| hs132676589 | TATTGTTCAGCCACAGGCTAAGG | 6460 |
| hs132676706 | GCAGTGTTTTGTGCAACAAACGG | 6461 |
| hs132676297 | AAACTTTTAAGGATATTTCAGGG | 6462 |
| hs132676615 | ATGTCATATGAATCTCTTCAGGG | 6463 |
| hs132676669 | TGTTGCTCAGCTTATCTGGCAGG | 6464 |
| hs132676682 | AGGATGTTAATAAGAAAGGAAGG | 6465 |
| hs132676052 | TTGGAGCTAGGTGGAAGTACTGG | 6466 |
| hs132676497 | GAGATATAGAAACCAAAAACGGG | 6467 |
| hs132676226 | ATTAATATATAATCATTTCAGGG | 6468 |
| hs132676387 | GCATATAATGTTAGATAAAGAGG | 6469 |
| hs132676196 | GTGTTGTGACAAGCACTCTATGG | 6470 |
| hs132676414 | GTAAGCAAGGAAATCATAAGAGG | 6471 |
| hs132676542 | CTGAGCTCTGGCCATTCCATAGG | 6472 |
| hs132676369 | TTAAAGCAAGAGGTATACTAAGG | 6473 |
| hs132676445 | TTTTATTTACAAGTTTTTGAAGG | 6474 |
| hs132676715 | AGACATAGACCTGACCTGATAGG | 6475 |
| hs132676087 | GTTGCCATGGACCTGACTCTGGG | 6476 |
| hs132676263 | AAGCATTTGCGGTTTTAACTTGG | 6477 |
| hs132676034 | AAATCAACCTGTTAAAGAAAGGG | 6478 |
| hs132676184 | TCTTCAATGCCTCACATTGACGG | 6479 |
| hs132676394 | GGCTTTTTAAATACTCACCTTGG | 6480 |
| hs132676642 | GTACCGCATTTTCTTTATGCAGG | 6481 |
| hs132676498 | TGAGATATAGAAACCAAAAACGG | 6482 |
| hs132676578 | AATTTCCTTTTAAAATTCACAGG | 6483 |
| hs132676037 | GAAAGGGGTAAAACATTTGAAGG | 6484 |
| hs132676554 | AATAAATCTCCCTATGAAGTTGG | 6485 |
| hs132676533 | GGAGTGGAAAACGGTATAATCGG | 6486 |
| hs132676631 | TTATGTCTAGCCTTGTGGTTGGG | 6487 |
| hs132676055 | TTAATTATAATGGCACTCCTTGG | 6488 |
| hs132676698 | ATTTTTACTTGGGGTATGAATGG | 6489 |
| hs132676513 | ATATGAAAACCCATGAGAAATGG | 6490 |
| hs132676303 | CATAATAATTTTCAATGCATGGG | 6491 |
| hs132676140 | CTAGGGGGTAATTAACTGATTGG | 6492 |
| hs132676108 | ACTGGGCTCAGACCTGATAAAGG | 6493 |
| hs132676518 | TTATAATTTGTGTATGTGGCAGG | 6494 |
| hs132676581 | TTTTTTCTAAAGTTGAGCTTGGG | 6495 |
| hs132676628 | CCGTTTCGCTGATTGTCAACAGG | 6496 |
| hs132676634 | TGGGCTCCTCTAGCCCCAAAGGG | 6497 |
| hs132676526 | CTGGTTGTCTCCAGAAGAATAGG | 6498 |
| hs132676453 | AGTACTTTCTTCTCAATAAGTGG | 6499 |
| hs132676357 | TTAAAAAGTTGGGGCTATATGGG | 6500 |
| hs132676390 | CAGTGACTATTTTCAATGATGGG | 6501 |
| hs132676392 | AGTCACTGAATTAGATTGAATGG | 6502 |
| hs132676681 | TTTTAGGATGTTAATAAGAAAGG | 6503 |
| hs132676520 | CCACATACACAAATTATAAACGG | 6504 |
| hs132676089 | GGAGACTTAGATCAAGTAATAGG | 6505 |
| hs132676347 | TGATCACTCTGATACATTGCAGG | 6506 |
| hs132676559 | AATCCATGAAGAAAAATTGATGG | 6507 |
| hs132676056 | GCAGTCAGCCTTAATTATAATGG | 6508 |
| hs132676721 | GATTGTGGCTAATATAATTCAGG | 6509 |
| hs132676167 | TCTCCACAAAAAGCTTTAAGAGG | 6510 |
| hs132676622 | ATATATGGCCCGTGGAGTTTCGG | 6511 |
| hs132676189 | CAGGGTGATGATTATTTAAGAGG | 6512 |
| hs132676130 | AAGCCCAAGGGGTAAGAAAAAGG | 6513 |
| hs132676276 | TGCTTTAGTCTAATTTCAATTGG | 6514 |
| hs132676179 | TCCCTCCTCTAGAGTCTTCATGG | 6515 |
| hs132676665 | AGATAAGCTGAGCAACATTTGGG | 6516 |
| hs132676057 | TCTGTTTTAGAATGAATGTTAGG | 6517 |
| hs132676661 | GAAACTGTGCAGCAATTAATTGG | 6518 |
| hs132676552 | TCTTGCACATCCAACTTCATAGG | 6519 |
| hs132676362 | CCTCCATATGTGAACATAATAGG | 6520 |
| hs132676380 | ATAAAGTGCAAATATTTTACAGG | 6521 |
| hs132676038 | GTTTTACCCCTTTCTTTAACAGG | 6522 |
| hs132676235 | CTTTTATCCCACTTTCCTTCAGG | 6523 |
| hs132676306 | ATGTACTTCCACCATCTATTTGG | 6524 |
| hs132676587 | GTCATAAGTATAAACAAAAATGG | 6525 |
| hs132676574 | ATATACCTGTGAATTTTAAAAGG | 6526 |
| hs132676195 | CACTTCACACAGATTTTAAAAGG | 6527 |
| hs132676142 | CTAGTGATCTGAATGCTTTCTGG | 6528 |
| hs132676522 | CTTCGTCGTAAACTTGTTAAAGG | 6529 |
| hs132676386 | TTAAGTTTCTGAATACTTAAAGG | 6530 |
| hs132675817 | AAGTAGAAGCAATAACAGCGAGG | 6531 |
| hs132675963 | TAAGTGGGAGAATATCTGCAAGG | 6532 |
| hs132675764 | AAGTAATGAAAATAGAACCAAGG | 6533 |
| hs132675934 | CTACTTCACAACAGTAAGCGGGG | 6534 |
| hs132675834 | TTTAGAGACAGTGAGACCTGAGG | 6535 |
| hs132675941 | TTTGGAATAAAAATATATGGGGG | 6536 |
| hs132675797 | TTATGTACAGTGACTGCAGGAGG | 6537 |
| hs132675916 | AAGCAGTTTGGGGAGAGATGCGG | 6538 |
| hs132675880 | TAGATCTATCAGATAACGAGCGG | 6539 |
| hs132675849 | TTGGTAGGCAGGAATTTACAGGG | 6540 |
| hs132675859 | ATAACAAATCTTGCTAACAGTGG | 6541 |
| hs132675871 | GTTCAATGAGAAGTCCTCCATGG | 6542 |
| hs132675898 | AAAACAACAGGCTATAAGAGAGG | 6543 |
| hs132675986 | TAGTGGATATAAGTTCATGTTGG | 6544 |
| hs132675777 | TGAACCTTAACCTTCCCAGAAGG | 6545 |
| hs132675831 | GGTCTCACTGTCTCTAAAGGAGG | 6546 |
| hs132675977 | TGCCACTAAGATGGTAACCGTGG | 6547 |
| hs132675755 | ATAAAGAAAAAAAACAACAAAGG | 6548 |
| hs132675804 | AGCAGATAGTGGCTTAATGGTGG | 6549 |
| hs132675845 | TGTCTTGCACAGAAGTGACTTGG | 6550 |
| hs132675876 | CTATGTTATTAATAGTGCCATGG | 6551 |
| hs132675833 | GAGACAGTGAGACCTGAGGACGG | 6552 |
| hs132675931 | ACAACAGTAAGCGGGGAGAGGGG | 6553 |
| hs132675811 | TTTAACGTTATGCACATAGAGGG | 6554 |
| hs132675877 | GTATGTTGCATTAGAAAAAGGGG | 6555 |
| hs132675841 | CAGAGGAATAGAAATGAAGAGGG | 6556 |
| hs132675982 | ACGTATATATATACAATGAGTGG | 6557 |
| hs132675889 | TGCTTAATAGTTTTAGAACAGGG | 6558 |
| hs132675989 | GCAGAGGTCAAATAGACAGTCGG | 6559 |
| hs132675832 | AGACAGTGAGACCTGAGGACGGG | 6560 |
| hs132675896 | TCAAGGATATGGTGATTCAAGGG | 6561 |
| hs132675795 | GAGTAACTGACTAAATAAGTAGG | 6562 |
| hs132676020 | ACATTTGTTTCACACGTCAAGGG | 6563 |
| hs132675927 | GGCAACAAAGAATAGCTCATAGG | 6564 |
| hs132675802 | TTCCAACCGTAATTTAGCACAGG | 6565 |
| hs132675800 | GTCCTGTGCTAAATTACGGTTGG | 6566 |
| hs132675824 | ATCATTGAGCTGGTCCCCAAAGG | 6567 |
| hs132675964 | ATTTCTTATCACGATTAAGTGGG | 6568 |
| hs132675900 | GTGGAGGCGCTTTTGCAATGAGG | 6569 |
| hs132676018 | AAAAGCAGCACTATGGAGCAGGG | 6570 |
| hs132675930 | GGAGAGGGGAAGAGAAAGAGCGG | 6571 |
| hs132675984 | ATGTATATGTATACAATGAGTGG | 6572 |
| hs132676012 | CCTTAACATTTCAGACGACATGG | 6573 |
| hs132675972 | ACCATGGCCCAGAACAGGGGTGG | 6574 |
| hs132675728 | TTACATTTTTGACCTACATGTGG | 6575 |
| hs132675936 | TGCTACTTCACAACAGTAAGCGG | 6576 |
| hs132675738 | GAAGTTAAGTCTATTCTCAAAGG | 6577 |
| hs132675971 | GGGACCATGGCCCAGAACAGGGG | 6578 |
| hs132675892 | GAGATAAGTAAGCAGAGGCTGGG | 6579 |
| hs132675919 | GACACCATCACTATATGGAACGG | 6580 |
| hs132675787 | CTAAGCCTATTATTTAGGAGTGG | 6581 |
| hs132675935 | GCTACTTCACAACAGTAAGCGGG | 6582 |
| hs132675929 | GAGAGGGGAAGAGAAAGAGCGGG | 6583 |
| hs132676005 | TTTATTTTAATCACTACCAAAGG | 6584 |
| hs132675858 | GATTTGTTATTACAACCAGCTGG | 6585 |
| hs132675766 | CTATGAGACAATCAAATACATGG | 6586 |
| hs132675860 | ACTTGGCTCTGGCAACCAGCTGG | 6587 |
| hs132675788 | ATAATAGGCTTAGTTATCAAGGG | 6588 |
| hs132675924 | CAGCCCGTTCCATATAGTGATGG | 6589 |
| hs132675970 | AGGGACCATGGCCCAGAACAGGG | 6590 |
| hs132675961 | TGATACACCTCAAATAAAAGTGG | 6591 |
| hs132676004 | TAATCACTACCAAAGGAATGAGG | 6592 |
| hs132675854 | ACTTTTACTGGACAATTCAAGGG | 6593 |
| hs132675872 | CATTGAACTTGTGAAAACTCAGG | 6594 |
| hs132675947 | TTTTAATGAACGTGGTGGGGGGG | 6595 |
| hs132675917 | AGATGCGGTATGTTGCTTGCTGG | 6596 |
| hs132675974 | TATACCAAATGCCACTAAGATGG | 6597 |
| hs132675882 | TAATCTTTGCTGAAATTCAAGGG | 6598 |
| hs132675905 | TGGACTAGTTCAGATGGAGTTGG | 6599 |
| hs132675816 | AAGCAATAACAGCGAGGCTGAGG | 6600 |
| hs132675807 | TGAATCACATTCATCATTGGAGG | 6601 |
| hs132675956 | TGACTGCTGTTTTTACTGGTCGG | 6602 |
| hs132675783 | AATAAAAAGATGACTAACATTGG | 6603 |
| hs132675830 | TCAGGTCTCACTGTCTCTAAAGG | 6604 |
| hs132675995 | TTTTTAAGTCACCTGAGCATAGG | 6605 |
| hs132675794 | ACTAAATAAGTAGGTCAAGAAGG | 6606 |
| hs132675754 | ATATAGCTTGCGTTAAATGATGG | 6607 |
| hs132675981 | AACTTGAATTTAAACTTCCACGG | 6608 |
| hs132675759 | AGGAAATATTTATCCCTAAGAGG | 6609 |
| hs132675842 | GGAATAGAAATGAAGAGGGTAGG | 6610 |
| hs132675967 | TTTACTCTTTTGATAGTGAAGGG | 6611 |
| hs132675999 | ATAAAAGATTAGTAGAATATGGG | 6612 |
| hs132675949 | AGCCTCTCAAACTATTTTGAGGG | 6613 |
| hs132675979 | TTCCACGGTTACCATCTTAGTGG | 6614 |
| hs132675920 | ACACCATCACTATATGGAACGGG | 6615 |
| hs132675983 | CAATGAGTGGAAAATATTAGTGG | 6616 |
| hs132675911 | GCATGCTTGAGTTCTTCTGCTGG | 6617 |
| hs132675743 | CTTGTTCATTGAAAGCTAGAAGG | 6618 |
| hs132675798 | CCTGCAGTCACTGTACATAAAGG | 6619 |
| hs132675801 | CGGTTGGAATCAGCAGATAGTGG | 6620 |
| hs132675985 | CAATGAGTGGATAATATTAGTGG | 6621 |
| hs132675950 | TAGCCTCTCAAACTATTTTGAGG | 6622 |
| hs132675761 | CTACATATGTACACCTCTTAGGG | 6623 |
| hs132675965 | TATTTCTTATCACGATTAAGTGG | 6624 |
| hs132676002 | AATGAGGCTTTCTCAGTCTTGGG | 6625 |
| hs132675867 | GCATATTTTGTAACATAGGCAGG | 6626 |
| hs132675921 | ATATGGAACGGGCTGAAAATCGG | 6627 |
| hs132675799 | TTGTGTCCTGTGCTAAATTACGG | 6628 |
| hs132675770 | CTTCTTCCAGCATCTCATAAGGG | 6629 |
| hs132675739 | TTGAGAATAGACTTAACTTCTGG | 6630 |
| hs132675875 | AGTATGTTGCATTAGAAAAAGGG | 6631 |
| hs132675978 | GTTACCATCTTAGTGGCATTTGG | 6632 |
| hs132675954 | ACTTAGAATGAGTACATTGATGG | 6633 |
| hs132675973 | GCCACCCCTGTTCTGGGCCATGG | 6634 |
| hs132675775 | CTTTCCTTCTGGGAAGGTTAAGG | 6635 |
| hs132675910 | ATACAGTTCAGAGTGCCTTTTGG | 6636 |
| hs132675870 | GACAGTAGAAAAACAGTAATAGG | 6637 |
| hs132675918 | TATTTGACACCATCACTATATGG | 6638 |
| hs132675852 | TGAATTGTCCAGTAAAAGTATGG | 6639 |
| hs132675945 | ACATGGCTGCTGAATTTAATAGG | 6640 |
| hs132676013 | GGATCATCACGGTATAAAATAGG | 6641 |
| hs132675790 | ATAATCCACTCCTAAATAATAGG | 6642 |
| hs132675952 | GCATCAATCTGGTTTAGTTCTGG | 6643 |
| hs132675885 | AAGGGCTTCTACCATTTAATAGG | 6644 |
| hs132676010 | ATAGGTTCTGGTACATTTACAGG | 6645 |
| hs132675781 | GGCATTTGTTAACTTCTATATGG | 6646 |
| hs132675737 | TATTATGTCTCATTACTAATTGG | 6647 |
| hs132675992 | TCTATTTGACCTCTGCAAAATGG | 6648 |
| hs132675710 | CATGATCTGGAACCATACTGGGG | 6649 |
| hs132675704 | AATGGAACGTTAGATCAATGTGG | 6650 |
| hs132675598 | GCACAATTAGGAATGTCATGAGG | 6651 |
| hs132675643 | AGCAACCTGATTTAGAACAGAGG | 6652 |
| hs132675617 | GGTCAGGGACAGATAAAACAGGG | 6653 |
| hs132675605 | TGCCAAATACAAAAAGAAGGGGG | 6654 |
| hs132675550 | AAATGGGGAAGGTAAGACAAGGG | 6655 |
| hs132675700 | ATATGTATCCCATAGAACACTGG | 6656 |
| hs132675613 | TATTTTGACAGAGAAAGAGTGGG | 6657 |
| hs132675626 | AAAACGTTAGAGAGCCAACAAGG | 6658 |
| hs132675642 | AGGTTGCTGAGTAATTCACCTGG | 6659 |
| hs132675685 | GGCAAAGTTAGAATACAACCTGG | 6660 |
| hs132675556 | GGTTAACATTAAATGCTGTGTGG | 6661 |
| hs132675675 | GTGTGTGTATTTGAGAGAGAGGG | 6662 |
| hs132675515 | GGAAGATTCTAAGTATCAAGTGG | 6663 |
| hs132675691 | GTATCAATATGTTTGACACTGGG | 6664 |
| hs132675547 | AGTAAAAAAAGGACATAGGTGGG | 6665 |
| hs132675681 | CCATCATCAGATACGTAGTGTGG | 6666 |
| hs132675635 | AGCCACATGCTTAACACTAAGGG | 6667 |
| hs132675490 | TTGTCAATGAAAATTAAGCATGG | 6668 |
| hs132675661 | GGCATAGGAAAATGGTAGAGTGG | 6669 |
| hs132675644 | AATTCACCTGGAAACCCAGTCGG | 6670 |
| hs132675532 | GGGGTATTGGAAAATATGGAGGG | 6671 |
| hs132675639 | TCAATACAGTTAGATGAGAAGGG | 6672 |
| hs132675518 | GATTCTAAGTATCAAGTGGTGGG | 6673 |
| hs132675696 | CGTGAAAGTGGTTATAGTGGAGG | 6674 |
| hs132675517 | AGATTCTAAGTATCAAGTGGTGG | 6675 |
| hs132675536 | ATAAGCTTGTTAGCTGTTAGGGG | 6676 |
| hs132675486 | CCTATTTGACTGGAATAGTGTGG | 6677 |
| hs132675578 | AATAAGGTAAGTTAAGATTGGGG | 6678 |
| hs132675603 | TCTGCCAAATACAAAAAGAAGGG | 6679 |
| hs132675507 | ATGTTTTGTAAAATCTGAAGCGG | 6680 |
| hs132675634 | GGCCCTTAGTGTTAAGCATGTGG | 6681 |
| hs132675698 | ACATTAGCGGAGCGTGAAAGTGG | 6682 |
| hs132675568 | GGGATGAAACATAATATGGCTGG | 6683 |
| hs132675572 | CACATATTATTATAACCAGCAGG | 6684 |
| hs132675597 | TGTGCCAACGACAATCGCTATGG | 6685 |
| hs132675690 | CTGGGAATCCCAGTAAATCCAGG | 6686 |
| hs132675645 | ATTCACCTGGAAACCCAGTCGGG | 6687 |
| hs132675625 | ATTGAAGTCTTCCACTTGAAGGG | 6688 |
| hs132675610 | CAGGAGAGAGTATATAAATGAGG | 6689 |
| hs132675672 | CGTTTCATTTACCCAACCCAGGG | 6690 |
| hs132675659 | TCTAGAAGCAGGATTGGCATAGG | 6691 |
| hs132675649 | TGGGGTTAATTAAATCCAGCTGG | 6692 |
| hs132675552 | GTAAAACTAACCAACTATGAAGG | 6693 |
| hs132675637 | CTTCTCATCTAACTGTATTGAGG | 6694 |
| hs132675705 | ACATTGATCTAACGTTCCATTGG | 6695 |
| hs132675627 | CTAACGTTTTTCCCTTCAAGTGG | 6696 |
| hs132675665 | TTAGGCAATGCATTTAGCCCTGG | 6697 |
| hs132675680 | CCACACTACGTATCTGATGATGG | 6698 |
| hs132675525 | ATAATTTATCCCAACAGCACTGG | 6699 |
| hs132675699 | TGTTGAAACATTAACATTAGCGG | 6700 |
| hs132675669 | CCTGGGTTGGGTAAATGAAACGG | 6701 |
| hs132675524 | GCTGAGTTTGCATAAGGTGTTGG | 6702 |
| hs132675702 | TCTTAATTAGTTCATTCCAATGG | 6703 |
| hs132675686 | AACTTTGCCGAGAATGGTGTTGG | 6704 |
| hs132675541 | TCATTTTTTTCTGTAGGCAATGG | 6705 |
| hs132675652 | TTAATTAACCCCATCCCGACTGG | 6706 |
| hs132675573 | ACTTGTACTTTGGTTCCTGCTGG | 6707 |
| hs132675561 | ATTAAAAATCTGGTGATCCTTGG | 6708 |
| hs132675574 | ACTGTCAGCTACTTGTACTTTGG | 6709 |
| hs132675501 | AATCCAAGTGCAGGAAACTATGG | 6710 |
| hs132675496 | TTAAATAGAAATATTTTAAAGGG | 6711 |
| hs132675660 | GCAGGATTGGCATAGGAAAATGG | 6712 |
| hs132675567 | CCTGGGGATGAAACATAATATGG | 6713 |
| hs132675653 | GTCAGGTGTCTTGCTCCAGCTGG | 6714 |
| hs132675531 | CCTAACAGCTAACAAGCTTATGG | 6715 |
| hs132675611 | TATACTCTCTCCTGTCATATTGG | 6716 |
| hs132675559 | GGTTGCAAGTCAATAAATTATGG | 6717 |
| hs132674283 | AGGCTTCTAAGAAGTCACTGTGG | 6718 |
| hs132672277 | AAAGAAGTTGAACCTCATGGCGG | 6719 |
| hs132673128 | TGATTAAGCCAAAATCACAGAGG | 6720 |
| hs132675048 | GACAACGGCCAAAATCAGTGTGG | 6721 |
| hs132674950 | TTCACATCACGACCTCACTGAGG | 6722 |
| hs132672183 | TAAGGTACAACTGTGTACGGGGG | 6723 |
| hs132674056 | CCACCCAGTAGATTTCCATGTGG | 6724 |
| hs132672163 | AGATTGTACATAGAAAACTGAGG | 6725 |
| hs132672089 | CTACGGATAGAAGTAGACCAAGG | 6726 |
| hs132673075 | TCACGTCTTGAAGCACCAGGTGG | 6727 |
| hs132675182 | TTTAACAAATCAGCCCCTAGGGG | 6728 |
| hs132672199 | AAGCATGATAAGAATCATGGAGG | 6729 |
| hs132672364 | TTGTTACAAGTAGAAAACAGTGG | 6730 |
| hs132673420 | GTGTAAGATTGTGTCTACAGTGG | 6731 |
| hs132672437 | GTTATACTTGATACACAGTGAGG | 6732 |
| hs132674934 | TCTGCTGTTTAACTACAGTGAGG | 6733 |
| hs132674077 | TAGCAGATCTAGCAGCTCTGGGG | 6734 |
| hs132673921 | ATATATGAGAGAAATCAACAAGG | 6735 |
| hs132674282 | TATGCATCACCTCTACCACAAGG | 6736 |
| hs132674043 | AATGAGCAGATTATATACTGGGG | 6737 |
| hs132673572 | TATGTACCTAGGACCTAATGGGG | 6738 |
| hs132672367 | TGCTTGCCTAGAGCATAACAGGG | 6739 |
| hs132673305 | TATTGGCATTCACATCCCAGTGG | 6740 |
| hs132672482 | TAACTGAAAACTGAAACCAGTGG | 6741 |
| hs132672548 | CTAAGCGTCTGCATACCTGGGGG | 6742 |
| hs132673330 | GCTCATCTGGACAATCACAAGGG | 6743 |
| hs132672280 | TTTGCCTTCAGACCGCCATGAGG | 6744 |
| hs132673242 | ACTGGACATAGCTAATCACACGG | 6745 |
| hs132673250 | GCAGATGTGTGAACATCTGGAGG | 6746 |
| hs132674112 | AAGCTGTTCATTGTGTACAGCGG | 6747 |
| hs132675207 | ATAACATCAAGAGAACACACTGG | 6748 |
| hs132672827 | TTTTTTGCTGAAACTAAACGAGG | 6749 |
| hs132674853 | TCAAACCTAAGGATGGTGGGGGG | 6750 |
| hs132675364 | GAAAACGAGACCATAAGCAGTGG | 6751 |
| hs132673156 | AGAACATCCATAATAACACAAGG | 6752 |
| hs132674072 | GCTACTCTCCTCCCTCAAAGCGG | 6753 |
| hs132673886 | ATTGCAGTAAGAGTAAATGGGGG | 6754 |
| hs132673203 | ATGTGGTTGAAATGTGACCATGG | 6755 |
| hs132674317 | TCTGTAAGATAAAAACATGGAGG | 6756 |
| hs132674407 | TATTTATATAGAGCACTAGGAGG | 6757 |
| hs132672475 | TATGTGGGATGGTGTCATGGTGG | 6758 |
| hs132672590 | CCTGATGACCAGAAACCCAGGGG | 6759 |
| hs132673370 | AACCACAGAATGCTTTACAGAGG | 6760 |
| hs132672631 | TGTGGTGCTGTAGAGATATGGGG | 6761 |
| hs132673371 | CAGACCTGCCCTTATCTTGGGGG | 6762 |
| hs132673675 | GCAGTGCAGAGGGTTTGTGGTGG | 6763 |
| hs132674316 | ACTTCTGTAAGATAAAAACATGG | 6764 |
| hs132674687 | GTAAGTGTGTGTACTATGAGGGG | 6765 |
| hs132675166 | TAATGAGAAATGCAGCCATGAGG | 6766 |
| hs132673353 | AATGCCAAGAAAAGTGACCGTGG | 6767 |
| hs132675036 | GACTACTATTAAATTAACTGGGG | 6768 |
| hs132673668 | GGTTTCCAAAACCTGACACAGGG | 6769 |
| hs132672948 | ATTTGAATATAGACCTCACATGG | 6770 |
| hs132675399 | ATACTGCAAACTAAGGACGTAGG | 6771 |
| hs132672125 | AGTGGGATAGTAATAGTTTGGGG | 6772 |
| hs132672299 | TGGCTAATCGGACTTCCCTGAGG | 6773 |
| hs132674330 | AATGGGTACAGAACACATGGTGG | 6774 |
| hs132672887 | ATAAACTATTTATGACACTGCGG | 6775 |
| hs132674526 | AGTAATGGGACTAAGACCGGAGG | 6776 |
| hs132675438 | GCCTTGAGAATTGAGAACAGAGG | 6777 |
| hs132672775 | TAGGTGTTACGAGTGTTGAGTGG | 6778 |
| hs132675212 | ACGTAAATGTTATTTCCCAGTGG | 6779 |
| hs132673682 | GTTGGAGTATACTCCCTGTGTGG | 6780 |
| hs132674626 | GTGCCAGGTAAGAAGCGTAGAGG | 6781 |
| hs132674061 | GATGCCATAAATGACTGGAGTGG | 6782 |
| hs132672889 | AAACTATTTATGACACTGCGGGG | 6783 |
| hs132673855 | ATACAGTGCAAAATACAAAGAGG | 6784 |
| hs132672219 | GTATTCTTAGGTTTTGCGGGGGG | 6785 |
| hs132672877 | ACTATGATCCATGATATGAGAGG | 6786 |
| hs132675449 | AATGAGTAACCATCCAACAGAGG | 6787 |
| hs132673633 | TTAAAGAGATTATAGCAACTGGG | 6788 |
| hs132672665 | TAAGGTGATATAAAACCTAGAGG | 6789 |
| hs132674169 | CTATTACTATGCCTATAATGCGG | 6790 |
| hs132673937 | ATATAGAAATGCATCTAAGGGGG | 6791 |
| hs132674756 | AGACGAATGTAGTATGTCTGGGG | 6792 |
| hs132672319 | TGAGCATCAACCTCCCCACAGGG | 6793 |
| hs132672208 | ACTCTGGCAAAGTTTCAGAAGGG | 6794 |
| hs132674929 | AAAAGAGACAGCCAGAAGTGGGG | 6795 |
| hs132675434 | AGTTGGAAAAGTGAAATCGGTGG | 6796 |
| hs132673980 | AGTGGAATAGTAATAGTTGGGGG | 6797 |
| hs132674255 | TAAGAAAATCGTGGTCCCAGTGG | 6798 |
| hs132672306 | CTCTTCTTTAGATTTCCCAGCGG | 6799 |
| hs132673050 | GTAGGAATAGAGCCTACCAGTGG | 6800 |
| hs132672257 | TGTTAAGAAACTCAATACAGAGG | 6801 |
| hs132673979 | GTGGAATAGTAATAGTTGGGGGG | 6802 |
| hs132674779 | TTAAATATGAAATAGTAACATGG | 6803 |
| hs132674733 | TAGGCCAGCAGTACTATGGTGGG | 6804 |
| hs132674994 | ATATCTTAAATTCCTCAGAGTGG | 6805 |
| hs132673317 | TAGGTAAGAGGAAAAATGCGAGG | 6806 |
| hs132673866 | TGGAAGGCAAGCCTGTAGAGAGG | 6807 |
| hs132674337 | TTTTGTTTAGAAAGAACATGGGG | 6808 |
| hs132672555 | ATAGTTTTAGTGAGATGACAAGG | 6809 |
| hs132674488 | ACTAAGAAAGAGCACTCCAGAGG | 6810 |
| hs132674123 | CTTTAGTGTATTCATATGTGAGG | 6811 |
| hs132673403 | CCAGCTAGTAAGCACACTCAGGG | 6812 |
| hs132673077 | ACTTCACGTCTTGAAGCACCAGG | 6813 |
| hs132672584 | AGGTAGAGCTCTACTAGCAGGGG | 6814 |
| hs132672323 | TTCCTAATACAAACCCTGTGGGG | 6815 |
| hs132672881 | AGAAATTGCATATATTCCAGGGG | 6816 |
| hs132672186 | ATAGGCATTTCCAAAGACTGTGG | 6817 |
| hs132672615 | GGAAAGCTTTCCTAATCAAGGGG | 6818 |
| hs132674392 | TATTTTGGAGAGTCATCACAGGG | 6819 |
| hs132673209 | CTTCTCTTAAGATTCCACAATGG | 6820 |
| hs132674519 | AATGGCCTGCTAAAGGGCAAGGG | 6821 |
| hs132675183 | ATAATTTCTATCTCCCCTAGGGG | 6822 |
| hs132672426 | GCTTAAGTGATTGTCACCTGCGG | 6823 |
| hs132672486 | TATTTTCTAAGCACTACTGGAGG | 6824 |
| hs132673012 | CTATATGACCATGGTCCAGGAGG | 6825 |
| hs132674195 | GAGTATGATCTTCTATAACACGG | 6826 |
| hs132672749 | ATATGTTGCCAAGAAATCAGAGG | 6827 |
| hs132674840 | TCAAAAAGGTTAAGAACCCGAGG | 6828 |
| hs132673006 | GTAAAAACGTTGTCTCAGCAGGG | 6829 |
| hs132672657 | GTAGGAGTGTTATCATACTGAGG | 6830 |
| hs132673342 | TTCTGAAAATCATAAAGACAGGG | 6831 |
| hs132673603 | CATTGCATACCATTTAGCTGGGG | 6832 |
| hs132674331 | ATGGGTACAGAACACATGGTGGG | 6833 |
| hs132673051 | TAGGAATAGAGCCTACCAGTGGG | 6834 |
| hs132675041 | TTTGTCCTATGTGACAACAATGG | 6835 |
| hs132675211 | AGTGGCCTAAAAGTTAGAAGAGG | 6836 |
| hs132673560 | AGTTAGTACCCATAGAGGTGAGG | 6837 |
| hs132673314 | CCACCAGCTGAGTCTATCTGTGG | 6838 |
| hs132673218 | TATTGTCACATAAATAAGAGAGG | 6839 |
| hs132674221 | GTAAAACTAAGAAACGTCAGTGG | 6840 |
| hs132672065 | AGTCAGATCAACTTAATGTGCGG | 6841 |
| hs132673519 | TGTTTGGGACAGCTACTCCAGGG | 6842 |
| hs132673885 | TTGCAGTAAGAGTAAATGGGGGG | 6843 |
| hs132673000 | TTTTTAACAAAAACTTACAAGGG | 6844 |
| hs132674707 | TATGCTAAAACATATACCTGTGG | 6845 |
| hs132672972 | AACTGAAGAAGGCCTGACGTTGG | 6846 |
| hs132673380 | AAATAGTATTGAGTGATGGGTGG | 6847 |
| hs132672448 | ATGGTATAAGGGCAGTCCTGGGG | 6848 |
| hs132674527 | GTAATGGGACTAAGACCGGAGGG | 6849 |
| hs132672990 | AAGCTGAATGATTCTCCAAGAGG | 6850 |
| hs132672383 | AGCAGGGATACAGTGGCAAGTGG | 6851 |
| hs132672191 | AACTGACAATAAAACAAAGGAGG | 6852 |
| hs132672531 | ATTCTTAACTCACAAGAGAGTGG | 6853 |
| hs132672518 | TGTCTTACTCTACATTCAGGGGG | 6854 |
| hs132672553 | TAAAACTATGAGTCAGCGGTGGG | 6855 |
| hs132672054 | TTCTTTACTGAGAACCAAAGCGG | 6856 |
| hs132674340 | TCTTCAAACACTAATTGTGGTGG | 6857 |
| hs132673530 | TGACTATTCATGTGACACAAAGG | 6858 |
| hs132672607 | CCAAGATCAGAGCCCATATGGGG | 6859 |
| hs132673946 | TTTCACTGCTCAGCAATATGCGG | 6860 |
| hs132672496 | TTGGGAGGTCCTAATTACAATGG | 6861 |
| hs132672845 | CATGTAGCTCACATATCCCATGG | 6862 |
| hs132674262 | TCATAAATAAAACTATCTGGTGG | 6863 |
| hs132672410 | TAAACTTAGGACAATTGTGACGG | 6864 |
| hs132672218 | TACGCTCCTTTCAGAGACTGTGG | 6865 |
| hs132675261 | ACTTCCTGAGAATGGTGACACGG | 6866 |
| hs132672586 | GCTAGTAGAGCTCTACCTGAGGG | 6867 |
| hs132673738 | TGGGAGACAAGCAGATACATGGG | 6868 |
| hs132673526 | GGCCTGCATCAGAGTCATCAGGG | 6869 |
| hs132674251 | TTAGAAATTATAATCCCACTGGG | 6870 |
| hs132673304 | ATTGGCATTCACATCCCAGTGGG | 6871 |
| hs132672463 | ATTCCTAAGGAAGAAACGAGTGG | 6872 |
| hs132672283 | AGAATGAGGTTAAGGTACCATGG | 6873 |
| hs132672697 | AGTACATAAATAAAGACATGAGG | 6874 |
| hs132672175 | GAAAACTTGAGAGAAATAGGAGG | 6875 |
| hs132674671 | GAACTGATAACAATGTTGCTCGG | 6876 |
| hs132675089 | AATTAGTAACAAGATGAGAGAGG | 6877 |
| hs132675340 | TGGTGGTTTCCCTAACACAATGG | 6878 |
| hs132674013 | TATGAATTATGGTTGCAGGAGGG | 6879 |
| hs132674843 | AATTGGCCTATAATACACCTCGG | 6880 |
| hs132674256 | ATTCTATGTTAAGAAAATCGTGG | 6881 |
| hs132672835 | TTGTCTAGGCCCTTACCAAGAGG | 6882 |
| hs132673015 | TAAGTATACCTCCTGGACCATGG | 6883 |
| hs132673985 | CATCTCCACATACATAGCAGTGG | 6884 |
| hs132673865 | ACTGAACTCTTAGAGAGCAGTGG | 6885 |
| hs132675055 | GCATGTGGAAATCTGTATGTTGG | 6886 |
| hs132672519 | GTGTCTTACTCTACATTCAGGGG | 6887 |
| hs132673920 | TATATGAGAGAAATCAACAAGGG | 6888 |
| hs132674037 | AAATCCCTGGATAGGCCAGGTGG | 6889 |
| hs132675397 | TGTTTTCTCTTAGTTCAGTGAGG | 6890 |
| hs132673497 | GAATCATAGACATACTTCAAGGG | 6891 |
| hs132674894 | AGACTAAGAATAAAAAGTAGGGG | 6892 |
| hs132673049 | GCCATGAGCAGAAAAAGAGTAGG | 6893 |
| hs132674463 | GACTCTTGCTTGATATAGGGGGG | 6894 |
| hs132674070 | TAGCACTCCTCAGTACTACCAGG | 6895 |
| hs132673010 | GCCTCTGCTCTATATGACCATGG | 6896 |
| hs132672799 | TCACATTGTTTGCCCCCATGGGG | 6897 |
| hs132675238 | GATATCAATTGGCTGAACAAGGG | 6898 |
| hs132672161 | TAGCGATGTCTTGGCAACAGTGG | 6899 |
| hs132673129 | GATTAAGCCAAAATCACAGAGGG | 6900 |
| hs132672405 | TTCCTGTTGATTAGGGCACACGG | 6901 |
| hs132674462 | TATAGGATGATCTAAGAACTCGG | 6902 |
| hs132675274 | ATGTTCTTACTTATTTACCAGGG | 6903 |
| hs132672910 | TTGATTTTACTTCAACCTAGAGG | 6904 |
| hs132672260 | AAGACAGTGACTAAAGGCAATGG | 6905 |
| hs132674032 | TCTCTTACCATAAACCCACCTGG | 6906 |
| hs132672384 | TCAATGAAGCAGGGATACAGTGG | 6907 |
| hs132674446 | TTCTTTCTACGTTTACAAGGGGG | 6908 |
| hs132674613 | ATGAATGTGCTCATGTGCATGGG | 6909 |
| hs132673232 | ACACCTTCCTATATCTGGGTAGG | 6910 |
| hs132673661 | CCTAACTTCATATGAAACACGGG | 6911 |
| hs132672164 | GATTGTACATAGAAAACTGAGGG | 6912 |
| hs132672109 | ACACAGCAGTGAATAGGCACAGG | 6913 |
| hs132674741 | AATCAAAGTGAAACATGAGAAGG | 6914 |
| hs132672282 | TGGCAGTGTAGTGCAATGACTGG | 6915 |
| hs132674991 | ATTTCTAGCAAGTCTCCTGAAGG | 6916 |
| hs132674276 | GAGCACACATCCCCTTCATGTGG | 6917 |
| hs132673731 | CATGCATTGAACCAAAACTCAGG | 6918 |
| hs132674346 | CTGGAAGACAAGCTAAGAGAGGG | 6919 |
| hs132672414 | GGTATGTAACTTCTAAACCCAGG | 6920 |
| hs132672670 | CCTTCAACTGTGGAAACCCAAGG | 6921 |
| hs132673776 | GAAAACAAGTTGGTTACAGGAGG | 6922 |
| hs132674292 | AGAAGGTAGAGCCACAACAAAGG | 6923 |
| hs132673809 | GTTTCCCAAATTGATCCACAAGG | 6924 |
| hs132672264 | ATTTTATGAAATGAGAGCAGTGG | 6925 |
| hs132672478 | ATACGCAACATCAGAATAAGGGG | 6926 |
| hs132675290 | AGCACTCTCTTAATATAGGGAGG | 6927 |
| hs132672386 | GAAGGCAATTCAATGAAGCAGGG | 6928 |
| hs132674666 | CAGTAAAATCAATAATAAAGGGG | 6929 |
| hs132674725 | CATATTACATGAAAGTGTGAGGG | 6930 |
| hs132672128 | TGGAATTCAAAACTGCCAGTAGG | 6931 |
| hs132672778 | GTAACAACAATAAAGAATTGTGG | 6932 |
| hs132674094 | AATGAAACTCTATCAACCGATGG | 6933 |
| hs132673669 | TGGTTTCCAAAACCTGACACAGG | 6934 |
| hs132673282 | GTGTACGGCACCATAGTCAAGGG | 6935 |
| hs132672325 | GTTTCCTAATACAAACCCTGTGG | 6936 |
| hs132673676 | CCACTCATAGTAAGCCACACAGG | 6937 |
| hs132674812 | GACATGGTTGATTCATGGAGCGG | 6938 |
| hs132672893 | GCCTAACGTTTACTAGATGTAGG | 6939 |
| hs132672614 | CGTTCCCATTGAGATAAGAGTGG | 6940 |
| hs132672467 | GGACCATTGGACAAACCAGATGG | 6941 |
| hs132674694 | TTACCATATAAATTACCAAGAGG | 6942 |
| hs132672724 | ATTGGTGCCTCTTTATATCGGGG | 6943 |
| hs132672431 | TTAGCGAGGAAGATAAGGGGTGG | 6944 |
| hs132674739 | ATTCTCACTTAATGTTCCAGTGG | 6945 |
| hs132674663 | GCTACTGTGCCACCTATCAGTGG | 6946 |
| hs132672173 | AGTACAGAATGGATTGCTGGAGG | 6947 |
| hs132674699 | TCTGTCGGGCATAAGAATGTCGG | 6948 |
| hs132673463 | TCTTAATAAAAGTTTTCTCGAGG | 6949 |
| hs132672295 | ATCTTGGGCAGTCAACCTCAGGG | 6950 |
| hs132674464 | GGACTCTTGCTTGATATAGGGGG | 6951 |
| hs132672651 | TATGGTCAGGACATTCACTTGGG | 6952 |
| hs132675028 | GGCAGCATCAGATTAACCTAGGG | 6953 |
| hs132674868 | TCGATAAGATGATTTTGGAGCGG | 6954 |
| hs132672355 | CAATGCTTCTCATTAAAGGACGG | 6955 |
| hs132673878 | ACAAGCCCCTTAACTGTCAGTGG | 6956 |
| hs132672251 | AATAAATGTTATGGACTTCGGGG | 6957 |
| hs132672213 | TGATGATAGCTTTAGAAGCGTGG | 6958 |
| hs132675414 | TGTATAGAAGCAATAAAAAGGGG | 6959 |
| hs132673013 | ACCATGGTCATATAGAGCAGAGG | 6960 |
| hs132673442 | ACACAGCACTCACAGAACTCAGG | 6961 |
| hs132674594 | CTGCGAAGTCCTGAATTCAGAGG | 6962 |
| hs132672730 | GTTACTCAACTAGAGAAGGTAGG | 6963 |
| hs132674874 | AGAACTACTGTATGGATCCGTGG | 6964 |
| hs132673938 | AATATAGAAATGCATCTAAGGGG | 6965 |
| hs132673811 | CAGATCTACACATAGATAGAGGG | 6966 |
| hs132673565 | TCTCCCATTGGGGACCTACGTGG | 6967 |
| hs132672220 | CGTATTCTTAGGTTTTGCGGGGG | 6968 |
| hs132674445 | TTTCTTTCTACGTTTACAAGGGG | 6969 |
| hs132672244 | TAATGTTTATAATTATGGGGTGG | 6970 |
| hs132674227 | ATTCTAAAGACAGCATCTGACGG | 6971 |
| hs132674509 | GTGCGAGTGTATCTTTCTAGGGG | 6972 |
| hs132674068 | TAATGTGCCCGCTTTGAGGGAGG | 6973 |
| hs132673758 | AAACACATTACCTGCCCACAGGG | 6974 |
| hs132674965 | AAACTAGAGCCAGAATAAGAGGG | 6975 |
| hs132675235 | TGTGATGATTACAGCAGAGGCGG | 6976 |
| hs132674051 | TTACATTGGCCATTTTTCTGGGG | 6977 |
| hs132674760 | TCTCCATGTATTGACAAGATGGG | 6978 |
| hs132672495 | GGAGGTCCTAATTACAATGGTGG | 6979 |
| hs132675336 | AAGTCTGAGACTTCAAGAGATGG | 6980 |
| hs132673974 | TTACAGTTAATAATTTCCTGAGG | 6981 |
| hs132672552 | CTAAAACTATGAGTCAGCGGTGG | 6982 |
| hs132673689 | CTATATGACAGAGTTGGCAGAGG | 6983 |
| hs132672735 | AATCATGATAGAGGATGCATGGG | 6984 |
| hs132674948 | ATCCACATATGACTCAAGGTGGG | 6985 |
| hs132672211 | ATTCTCAAACTAATTAACAATGG | 6986 |
| hs132673063 | CAACAGCAACAACAAAAGGGGGG | 6987 |
| hs132673285 | CATGACCATAGCCAAGTGTACGG | 6988 |
| hs132673253 | TAACTGACAGTGAGTGACAAAGG | 6989 |
| hs132673020 | CACGTTACATTTTAACAGTGGGG | 6990 |
| hs132672221 | GCGTATTCTTAGGTTTTGCGGGG | 6991 |
| hs132674234 | ATTGTATCAGAAATATCTAACGG | 6992 |
| hs132672524 | AAATTGAGCACTGCTTGAAGGGG | 6993 |
| hs132674127 | CATGCTCTAAACACCACTAAGGG | 6994 |
| hs132672739 | ATCAGATTATGTGGATGCAATGG | 6995 |
| hs132674848 | CCATTGCTCAAACCTAAGGATGG | 6996 |
| hs132672281 | GTCATTGCACTACACTGCCATGG | 6997 |
| hs132675234 | AAATGTGATGATTACAGCAGAGG | 6998 |
| hs132674609 | ATTGAGCAGCGATGTGCACAGGG | 6999 |
| hs132675233 | AAATGTTTATAAGTTTACACAGG | 7000 |
| hs132675467 | AAATGTGTATGCCCTATGGATGG | 7001 |
| hs132672129 | TTCCACATAATCTCAGAAGTGGG | 7002 |
| hs132674560 | TTACATCTAGGGATACAGACTGG | 7003 |
| hs132673295 | ATGCAGTGACAATATCAGAATGG | 7004 |
| hs132673485 | TTGCAAATCTGTCTAAACATGGG | 7005 |
| hs132673394 | ACTATGCTAGGCACAGGGCAGGG | 7006 |
| hs132675319 | GGGGAGAATTTTATTATTGGGGG | 7007 |
| hs132672432 | ATGTTAGCGAGGAAGATAAGGGG | 7008 |
| hs132674905 | AATGCCTCTTGATCAATCTGAGG | 7009 |
| hs132672542 | AGTGCTCTCAGACTGAGGATAGG | 7010 |
| hs132674491 | AGGTCAGACAGAATATGGATAGG | 7011 |
| hs132674726 | GCATATTACATGAAAGTGTGAGG | 7012 |
| hs132673390 | AAATATATAGAAACTATGCTAGG | 7013 |
| hs132672547 | GCTAAGCGTCTGCATACCTGGGG | 7014 |
| hs132672559 | CAATAAAGAAACAAAAACCTAGG | 7015 |
| hs132675392 | GTATAGCTCTTTATCCTCAGAGG | 7016 |
| hs132672472 | TCAGCACAAAACATTTATGTGGG | 7017 |
| hs132672210 | TCATTGAAGTGAATCCACTCTGG | 7018 |
| hs132674522 | ACTCTCTTATATTACAATCGTGG | 7019 |
| hs132674406 | ATATATTTATATAGAGCACTAGG | 7020 |
| hs132675287 | CCCCTGTGAATAACAAAGTGAGG | 7021 |
| hs132673907 | CATACAGATGAAGTAAAAGAGGG | 7022 |
| hs132673756 | ATAGATGAAAAGTTCCCTGTGGG | 7023 |
| hs132675123 | AATTAGAAAAATCTTTGCAGGGG | 7024 |
| hs132675296 | GTCTTACAGAGAAATCATAACGG | 7025 |
| hs132674814 | CATGGTTGATTCATGGAGCGGGG | 7026 |
| hs132673778 | GTTGGTTACAGGAGGAAGCAGGG | 7027 |
| hs132675255 | AACAACTCAAGAGTGATGCAGGG | 7028 |
| hs132674288 | TTCATATAGCCATTGAAGAGGGG | 7029 |
| hs132675444 | ATAGGCTAGAAGAAGAGTAGAGG | 7030 |
| hs132674947 | AATCCACATATGACTCAAGGTGG | 7031 |
| hs132673709 | ACAGAAGTCACAATGATAGTGGG | 7032 |
| hs132675073 | ATTTATTTACCATTTATCCAGGG | 7033 |
| hs132675325 | TGTTTCATTATTCTCCTAAGGGG | 7034 |
| hs132673427 | GTTTCAAAATGGACTTAAGGGGG | 7035 |
| hs132672420 | GGAAGCTACTCAGTCAGCATGGG | 7036 |
| hs132675343 | TTATGGCTGCAAATAAGATGAGG | 7037 |
| hs132674938 | TGTACTCTTGCCTCTTGAGGAGG | 7038 |
| hs132672263 | TTGAGGAGTATGAAAATGATGGG | 7039 |
| hs132674631 | AATATTCCCTGAAAACAGTAGGG | 7040 |
| hs132674634 | TGAATCACAAAAGGCACGAAGGG | 7041 |
| hs132675260 | CTCAGGAAGTCAAAATAAAGGGG | 7042 |
| hs132672322 | CTAATACAAACCCTGTGGGGAGG | 7043 |
| hs132673399 | TTTGTGTGTTTATGAGGACAGGG | 7044 |
| hs132672226 | GGTCAACCACAGTCTCTGAAAGG | 7045 |
| hs132673892 | AGCAAAACGACCACAAATAGGGG | 7046 |
| hs132673237 | TCTTGTATGTGCCGAGAAGGAGG | 7047 |
| hs132673415 | ATCTTCAGGGCAGTGATGCGGGG | 7048 |
| hs132673681 | CCTGTGTGGCTTACTATGAGTGG | 7049 |
| hs132674562 | CTAGATGTAAGATTCTGACCTGG | 7050 |
| hs132674108 | TTATTTCTCCTGCATTAATGGGG | 7051 |
| hs132674058 | ATTCCACATGGAAATCTACTGGG | 7052 |
| hs132673246 | AGAATCATGACTCTTTGAGAGGG | 7053 |
| hs132673662 | GCCTAACTTCATATGAAACACGG | 7054 |
| hs132672349 | AATGTTAAAAAATTTAACATGGG | 7055 |
| hs132673130 | ACAGAGGGTTACCAGTGACTTGG | 7056 |
| hs132675297 | ACAGTACATTCTACATGCGTTGG | 7057 |
| hs132672237 | ACAAATGGTCTCCTACAACTAGG | 7058 |
| hs132673079 | AAGTAATGTGATTTAGTGTGAGG | 7059 |
| hs132674529 | GTCCCATTACTTGAAAGAAGGGG | 7060 |
| hs132673289 | CTGTTACCAAAGGGAATGGGTGG | 7061 |
| hs132674076 | TCTGGGGCAACATTCTGCAATGG | 7062 |
| hs132674088 | GTCCTGACTAGCTCTCCTGCAGG | 7063 |
| hs132674147 | TAGCATACTGTTGTTGTGTGCGG | 7064 |
| hs132672925 | AATAATATCAATGCAAGCAGAGG | 7065 |
| hs132674542 | TAAGCATTTGAGAACATCAGTGG | 7066 |
| hs132674706 | GATATACAAGAAGCTGCAGAAGG | 7067 |
| hs132672317 | TAAACAACAAAAACTGTGAGAGG | 7068 |
| hs132675194 | AATATGATATTTGTGTGTGCGGG | 7069 |
| hs132673144 | TGATGGACTTTACCTAAGGTGGG | 7070 |
| hs132673523 | AGAGTCATCAGGGAAGGTGGTGG | 7071 |
| hs132674012 | TTATGAATTATGGTTGCAGGAGG | 7072 |
| hs132675228 | TGAGAGGGTTAATTCTCAGGAGG | 7073 |
| hs132673677 | CACTCATAGTAAGCCACACAGGG | 7074 |
| hs132673414 | TAATGAGCCAATGATCCTGATGG | 7075 |
| hs132673717 | TGTAATGTACAGAGAACTGAAGG | 7076 |
| hs132672427 | AAGCAAAGCACGAAAACCGCAGG | 7077 |
| hs132672498 | TTATTGTATTTGTAGTTGGGAGG | 7078 |
| hs132673164 | AATGAATCTTTCAAAAGGAGAGG | 7079 |
| hs132673863 | GCTAGAGCTAGGAAGTGAAAGGG | 7080 |
| hs132673329 | AGCTCATCTGGACAATCACAAGG | 7081 |
| hs132674235 | GCTTTATGAAACCCACGAGTGGG | 7082 |
| hs132674835 | GCCAATAAAATACATAAGGGAGG | 7083 |
| hs132674842 | ATTGGCCTATAATACACCTCGGG | 7084 |
| hs132673204 | GTGGTACTTTCGACTGTCCATGG | 7085 |
| hs132674775 | AATACACTCAGTATTCATGTAGG | 7086 |
| hs132672771 | ATCTGGTGATAAACGTTCAGAGG | 7087 |
| hs132673627 | CTAAAGCCAAGATTTGCTGAAGG | 7088 |
| hs132672685 | TGACAAGAAATAATCTAGAGAGG | 7089 |
| hs132672583 | CAGGTAGAGCTCTACTAGCAGGG | 7090 |
| hs132673971 | CTGAGGGACTACTTTCTGAGTGG | 7091 |
| hs132672094 | TTTCAATGCACAAGGCTGAAAGG | 7092 |
| hs132673951 | GCATATTGCTGAGCAGTGAAAGG | 7093 |
| hs132672435 | TAGACTATTTTATGTTAGCGAGG | 7094 |
| hs132674214 | AGATTTACAGACACATAGTAGGG | 7095 |
| hs132675114 | TAGAAGTCTAAACAAATGCTGGG | 7096 |
| hs132672886 | GCAATCTTAACTTTCTATGGAGG | 7097 |
| hs132673496 | AGAATCATAGACATACTTCAAGG | 7098 |
| hs132673286 | GTGTAACCAGGTCATCATAGTGG | 7099 |
| hs132675129 | TTTGTCTTATGGCAATCGTGTGG | 7100 |
| hs132674481 | TGAGTTGCTGTGACAGTCTGTGG | 7101 |
| hs132673876 | TCTTGGTCGTTGAATAACAATGG | 7102 |
| hs132674851 | GCTCAAACCTAAGGATGGTGGGG | 7103 |
| hs132675052 | CCATATAGCTGCTAGGACAACGG | 7104 |
| hs132674399 | AAGCACTGATCAAGAATGAGAGG | 7105 |
| hs132672098 | GTTTGTAAGATTATTAAGGAAGG | 7106 |
| hs132673760 | AACTAAGAGCAGCCCAACACAGG | 7107 |
| hs132674320 | TTCAGGAACATGTATTCCCAAGG | 7108 |
| hs132673198 | GGAAATTATTAACAGACCAGTGG | 7109 |
| hs132674435 | CAAGATGAATCTCCTTTGGAGGG | 7110 |
| hs132674298 | CTGCTCTTATATCTTTCCAAGGG | 7111 |
| hs132672873 | TGACAGTATGGCTTGAGAGAGGG | 7112 |
| hs132674389 | TGTTGTTATGCATCTTTCAGGGG | 7113 |
| hs132674421 | TGTCAATGCCTAACTGATGAGGG | 7114 |
| hs132673943 | TTAAGCTAGGAAAGAAAGTGGGG | 7115 |
| hs132674662 | TTCATCATTCCACTGATAGGTGG | 7116 |
| hs132674444 | TTTTCTTTCTACGTTTACAAGGG | 7117 |
| hs132673960 | TTTGCATATCTGAATTTCTGAGG | 7118 |
| hs132673293 | GGTGTGTTATCTGTTACCAAAGG | 7119 |
| hs132672837 | TTAACAGCTCCTCTTGGTAAGGG | 7120 |
| hs132672550 | CTTGAATCTTTCAAACCCCCAGG | 7121 |
| hs132672743 | TCAAATAGAAAGTATTACAAAGG | 7122 |
| hs132674228 | GGAATAGAGCTATTCATCCCTGG | 7123 |
| hs132672182 | CTAAGGTACAACTGTGTACGGGG | 7124 |
| hs132674085 | CTGTTAATGGAAAGTCTGGAAGG | 7125 |
| hs132673038 | ATAAGACTAAAAGAAAGGACAGG | 7126 |
| hs132674365 | ACCAAATGTACATTTGGAGAGGG | 7127 |
| hs132673913 | CATGTTTCGAAATAGAGAAGAGG | 7128 |
| hs132673933 | TTAATCCTGTAGACAAAATGTGG | 7129 |
| hs132675099 | ACTCAAACAGTTTCCAGCAAGGG | 7130 |
| hs132675473 | CTCTACCATACTAAAAGCAGTGG | 7131 |
| hs132672878 | TCTATAATATTGTAGTCCATAGG | 7132 |
| hs132673997 | TCTGCCTAACATTTATCATGAGG | 7133 |
| hs132675224 | TGAAATACCGTATATAGAGAAGG | 7134 |
| hs132675070 | CTGTTTAGCAAAACTAACCCTGG | 7135 |
| hs132675134 | ATAGGATTTATTCCTTGCAGAGG | 7136 |
| hs132673912 | ATGTTTCGAAATAGAGAAGAGGG | 7137 |
| hs132675446 | CTGTAATCTCTACGTCTGGAAGG | 7138 |
| hs132673806 | ATACAAGATTAATCTCCTTGTGG | 7139 |
| hs132675054 | ATGTGCTCCATATAGCTGCTAGG | 7140 |
| hs132672358 | TTAATAACGATAAAAAGTAATGG | 7141 |
| hs132673586 | AGTCTAGTAAATTTGGTAGGGGG | 7142 |
| hs132673690 | AGTGATCTATATGACAGAGTTGG | 7143 |
| hs132673812 | ATAGATAGAGGGACATTCCAAGG | 7144 |
| hs132673513 | AATCTTCCCAGGCAATTCTGAGG | 7145 |
| hs132675168 | TAATGTCACTGTATAGGCCAAGG | 7146 |
| hs132672847 | CATGGGATATGTGAGCTACATGG | 7147 |
| hs132672681 | AAATCACATGAATGGTAAGAGGG | 7148 |
| hs132674787 | ACTATACACACCAAGTCTGTAGG | 7149 |
| hs132675271 | CAAATTTGACCAAAAAAGGAAGG | 7150 |
| hs132672510 | CATGCTGTACTTTACAGGCATGG | 7151 |
| hs132672228 | TCATAACATCTTAAATAAGAGGG | 7152 |
| hs132673649 | AGAAATGTACTAGGTTTCATGGG | 7153 |
| hs132672958 | TCTTATCAGACATTTCACCTTGG | 7154 |
| hs132673273 | AATGTGTGTTCCATGTCAAATGG | 7155 |
| hs132674287 | TCATATAGCCATTGAAGAGGGGG | 7156 |
| hs132672156 | GGAAGAGTATTCGGGGCAGTGGG | 7157 |
| hs132675441 | CTACCATTTCAAAATTGGCATGG | 7158 |
| hs132673280 | TGGTGCCGTACACTTGGCTATGG | 7159 |
| hs132672159 | AGAATAAACATGCAGAGGTAGGG | 7160 |
| hs132675088 | TCACTGTGTTCTGCTATCTGAGG | 7161 |
| hs132673325 | CGAAGCATGAAATATTCAGAAGG | 7162 |
| hs132674746 | GGATTACTGGAGTAGAGGCAAGG | 7163 |
| hs132675471 | ATTCATAATATAGTGCAAGAAGG | 7164 |
| hs132672298 | AGTCCGATTAGCCAGTCGCTGGG | 7165 |
| hs132672596 | GATAGTTATACCAAGCCCCTGGG | 7166 |
| hs132673135 | TTTCTAGGCATCCAATAATGTGG | 7167 |
| hs132672589 | TCCTGATGACCAGAAACCCAGGG | 7168 |
| hs132673929 | TCATTTCTAATTTTTCTCTGGGG | 7169 |
| hs132674855 | CACCCATCTGTGCAAACGTGTGG | 7170 |
| hs132675400 | CTTAGTTTGCAGTATAGGGGAGG | 7171 |
| hs132673488 | GAGAATTCCATTCCTAACCTGGG | 7172 |
| hs132672729 | TATAGTTACTCAACTAGAGAAGG | 7173 |
| hs132674751 | ACATTCACAAGCAGCAGCAAGGG | 7174 |
| hs132672696 | ATAAATAAAGACATGAGGAAAGG | 7175 |
| hs132673369 | GGTTCCCCCAAGATAAGGGCAGG | 7176 |
| hs132673498 | TTCAAGGGATCCATGAACACTGG | 7177 |
| hs132672289 | TACTCACATTGACTTTCTTGGGG | 7178 |
| hs132672876 | AGGCAGAGAATGCTCAGCAGTGG | 7179 |
| hs132674606 | GGAAAGAGCTATCTTAAGGATGG | 7180 |
| hs132672706 | ACATTACCAAGTCCTGAGAAAGG | 7181 |
| hs132673791 | AATTTCTTGTTGAGCCGGAATGG | 7182 |
| hs132674954 | ACTCTAAAATTAGATGGCAAAGG | 7183 |
| hs132674089 | GGAGAGCTAGTCAGGACTCAGGG | 7184 |
| hs132675035 | GGACTACTATTAAATTAACTGGG | 7185 |
| hs132673784 | TAATCAGCAAGCTTAATCTAGGG | 7186 |
| hs132675083 | GAATTTTTAATAATAACTAAGGG | 7187 |
| hs132674625 | AATTGTCCCATGACTGTGCCAGG | 7188 |
| hs132675132 | TCTTCAGGTTTCCCTCTGCAAGG | 7189 |
| hs132674164 | TTTAATGGTATGTATTAATGCGG | 7190 |
| hs132675426 | AGGAGATCTTTCAATATGGATGG | 7191 |
| hs132673528 | TGTGCTTTGCTCTCAAAGTGTGG | 7192 |
| hs132673054 | GTTTTAATTTTCCCACTGGTAGG | 7193 |
| hs132672841 | GCTGTGATATACTTCACATTGGG | 7194 |
| hs132675230 | TACACAGGTTAAAATTGAGAGGG | 7195 |
| hs132674483 | TCTCGCTAGCTATAAATACCAGG | 7196 |
| hs132675180 | ACTTTAACAAATCAGCCCCTAGG | 7197 |
| hs132675012 | AGAGTCAGCATTTATTCTAGAGG | 7198 |
| hs132672660 | TGTTTAATGGTCTTTCACAATGG | 7199 |
| hs132675273 | TTTTTGTTCTAAAGTTACCCTGG | 7200 |
| hs132672744 | TAATAGAGCTAAGCTTCTAGAGG | 7201 |
| hs132673587 | CAGTCTAGTAAATTTGGTAGGGG | 7202 |
| hs132673239 | GTATGTGCCGAGAAGGAGGCTGG | 7203 |
| hs132674505 | TGATAGGAACATAGGAAAGTCGG | 7204 |
| hs132672254 | TAGTGCAGTCATCATAAATGAGG | 7205 |
| hs132672279 | CGGTCTGAAGGCAAATTCAAAGG | 7206 |
| hs132675263 | GGATTAAAACCAAACCCTAACGG | 7207 |
| hs132672540 | AAATACGTTTTTGTAAAGCACGG | 7208 |
| hs132674279 | CCTTCACTTTCCACATGAAGGGG | 7209 |
| hs132674910 | AGATTAACGTGAAGAAGGCTAGG | 7210 |
| hs132675384 | TTACTGTTTTATGAAAGGATAGG | 7211 |
| hs132672345 | TGTAGACCATATCATGGCAATGG | 7212 |
| hs132673595 | TGCTGAGTCACAGTTCTCAAGGG | 7213 |
| hs132673372 | ACAGACCTGCCCTTATCTTGGGG | 7214 |
| hs132674852 | CTCAAACCTAAGGATGGTGGGGG | 7215 |
| hs132673680 | TACTATGAGTGGCAGTGCAGAGG | 7216 |
| hs132673975 | TTCTGAAGCAGAAAAATACATGG | 7217 |
| hs132674253 | AAGAAAATCGTGGTCCCAGTGGG | 7218 |
| hs132672628 | AAAATATCAAAGACAACTAACGG | 7219 |
| hs132673684 | AAGCAAGCAAGCAATTAAGAAGG | 7220 |
| hs132674145 | CACACAACAACAGTATGCTATGG | 7221 |
| hs132675374 | CCTATTTTCTGATTATGATGTGG | 7222 |
| hs132675109 | GACTAGCTGTCACATAAGACTGG | 7223 |
| hs132672115 | GGGGCAATAAAAATAATGCTTGG | 7224 |
| hs132672304 | CGCTGGGAAATCTAAAGAAGAGG | 7225 |
| hs132672479 | ACAACTGACAAAATAACTGATGG | 7226 |
| hs132675420 | CTTGATAAGCACATGTACTTGGG | 7227 |
| hs132674093 | ACTCAGTAACACTCTCCTGCAGG | 7228 |
| hs132675284 | TCCTCACTTTGTTATTCACAGGG | 7229 |
| hs132672398 | GCAAATGCACAGAAGCTGGTGGG | 7230 |
| hs132674952 | AGTTAACTGAGAAAGAGTAGAGG | 7231 |
| hs132672872 | TTGACAGTATGGCTTGAGAGAGG | 7232 |
| hs132674857 | GTCCACACGTTTGCACAGATGGG | 7233 |
| hs132673290 | TATCTGTTACCAAAGGGAATGGG | 7234 |
| hs132675405 | CTCCCTTAGAGCTAAAAACAGGG | 7235 |
| hs132675453 | ATTACAACAGAAGTAGTGGCAGG | 7236 |
| hs132673911 | TTCGAAATAGAGAAGAGGGATGG | 7237 |
| hs132674374 | GACTCATCTTAATTTTCACATGG | 7238 |
| hs132674677 | TAAAAATGTCTAAATGCTAGTGG | 7239 |
| hs132674809 | CTGACATTAATAGCTTGGATCGG | 7240 |
| hs132673429 | ATTTTGAAACAAACTTAGCAAGG | 7241 |
| hs132672652 | TTATGGTCAGGACATTCACTTGG | 7242 |
| hs132672324 | TTTCCTAATACAAACCCTGTGGG | 7243 |
| hs132674623 | TGTTGAACCATATGAAGCCCAGG | 7244 |
| hs132672588 | TTCCTGATGACCAGAAACCCAGG | 7245 |
| hs132673445 | GAGAAAGATGGAAGCTAAGGAGG | 7246 |
| hs132674459 | TATTAACTCTTTAGGATACAGGG | 7247 |
| hs132673730 | GAACCAAAACTCAGGCCGTAAGG | 7248 |
| hs132673428 | GAAACAAACTTAGCAAGGAGTGG | 7249 |
| hs132675191 | ATAAAGGGTAAATTCAAGCCAGG | 7250 |
| hs132672447 | GATGGTATAAGGGCAGTCCTGGG | 7251 |
| hs132675029 | AGGCAGCATCAGATTAACCTAGG | 7252 |
| hs132673408 | TTTTAGAAGAGATTACCATCAGG | 7253 |
| hs132674670 | ACAGAGAGTCATTACTAGATGGG | 7254 |
| hs132672169 | GTAAATACTTTTCAATGAGAGGG | 7255 |
| hs132672616 | GGGAAAGCTTTCCTAATCAAGGG | 7256 |
| hs132674062 | CATCTGACTTTGATTCCACATGG | 7257 |
| hs132672914 | TCTCTAATCTGCACTAGAGAAGG | 7258 |
| hs132673813 | TCTATCTATGTGTAGATCTGTGG | 7259 |
| hs132673576 | CAAAAAATAGAAGCCAATGTGGG | 7260 |
| hs132672180 | TCTAAGGTACAACTGTGTACGGG | 7261 |
| hs132673062 | CCAACAGCAACAACAAAAGGGGG | 7262 |
| hs132673029 | GGACAGAAGAGTGTCTTCCTAGG | 7263 |
| hs132674270 | ACTGCATTATCCTTGTAGACAGG | 7264 |
| hs132672960 | TCACATTTTCTCATAGGCCAAGG | 7265 |
| hs132673569 | TTGCCACGTAGGTCCCCAATGGG | 7266 |
| hs132672259 | TTATGTAAGACAGTGACTAAAGG | 7267 |
| hs132674240 | CCCCATTCATATGTCATGAAGGG | 7268 |
| hs132674233 | TACAATCTTTGCCCACTCGTGGG | 7269 |
| hs132672193 | AAGGAGGAAGAGGACGAAAGAGG | 7270 |
| hs132673043 | GAATCTTTAATATAGGTAGTGGG | 7271 |
| hs132673699 | AAGAGATCATACTTTTACTGTGG | 7272 |
| hs132675133 | TAGGATTTATTCCTTGCAGAGGG | 7273 |
| hs132672875 | GGCAGAGAATGCTCAGCAGTGGG | 7274 |
| hs132672044 | TGAAAGCTACTCTGTTAGATGGG | 7275 |
| hs132674111 | ATAAATGACCCCATTAATGCAGG | 7276 |
| hs132673153 | AATTGAATGGAGGTAAAACTAGG | 7277 |
| hs132673299 | ACATTGGCTTTATTTTAGAACGG | 7278 |
| hs132674171 | TTAGCGGTGTTGCTTTTGGTTGG | 7279 |
| hs132673774 | CAGAGTAAGTGAAAACAAGTTGG | 7280 |
| hs132675060 | TGTACAGAAGGATACAAGGCAGG | 7281 |
| hs132675165 | TATTTAGCATTAGAACCTCATGG | 7282 |
| hs132672419 | TGGAAGCTACTCAGTCAGCATGG | 7283 |
| hs132673867 | GAACTTTGAAAGGTTATGGAAGG | 7284 |
| hs132674137 | AGTAATAGTAAATGAGGCTGGGG | 7285 |
| hs132674901 | TAGAGCACGAGCATTTGTGCAGG | 7286 |
| hs132673465 | ATGATTCATTACTATAAGCAGGG | 7287 |
| hs132673412 | TTATCTTCAGGGCAGTGATGCGG | 7288 |
| hs132672541 | ACTCAAGTGCTCTCAGACTGAGG | 7289 |
| hs132674897 | ATGTGGTCAAATCAACCCATTGG | 7290 |
| hs132674591 | ATTCTAATCACCCCATCTGAAGG | 7291 |
| hs132672348 | GTTAAAAAATTTAACATGGGAGG | 7292 |
| hs132672767 | CATTGTGTACCAAGCTCTTGAGG | 7293 |
| hs132672084 | AAGTCCGTTAGCACAAAAGCAGG | 7294 |
| hs132673767 | TTTAACTTATATTTTATACCTGG | 7295 |
| hs132674930 | GAAAAGAGACAGCCAGAAGTGGG | 7296 |
| hs132674453 | TATATTAGTTCACTTACATAGGG | 7297 |
| hs132673214 | ATTTATGGTATCTATGGAAGAGG | 7298 |
| hs132672953 | GGCTGAAGTCATTTAATCCATGG | 7299 |
| hs132675401 | GTCCTTAGTTTGCAGTATAGGGG | 7300 |
| hs132674973 | AGGTTACTCATTTTATTACAGGG | 7301 |
| hs132673495 | GTCTTTGAGTAACATATCCCAGG | 7302 |
| hs132674992 | TCACAGTTGTCATGGATACATGG | 7303 |
| hs132674504 | ACATTAAATGATAGGAACATAGG | 7304 |
| hs132672121 | TTTCATTTACATAAAACATGTGG | 7305 |
| hs132674468 | CCAAGCATAATTACCCGTGATGG | 7306 |
| hs132673786 | AGTTACTGCAACTGAATGACAGG | 7307 |
| hs132673934 | AATGTGGAAGTAACAAAGACAGG | 7308 |
| hs132675464 | TGCAGTATGCTCCATCCATAGGG | 7309 |
| hs132675104 | AGAGAGACAACTGTGCAGACAGG | 7310 |
| hs132672802 | GGCGTTAAAAAAATCCCCATGGG | 7311 |
| hs132675150 | ATAATTATAAGTGATATCACAGG | 7312 |
| hs132672908 | AAGGCAGTCATGGTTCCTCTAGG | 7313 |
| hs132673881 | TATGACCACTGACAGTTAAGGGG | 7314 |
| hs132674735 | ATGGTGGGAACAAACTCCACTGG | 7315 |
| hs132674411 | AGTCAGAGTATCAAAATCACTGG | 7316 |
| hs132673691 | TCTATGGTATCGGGATAAGATGG | 7317 |
| hs132674100 | GAAGTTACTGCAATATTAGGAGG | 7318 |
| hs132673065 | ACAGCAACAACAAAAGGGGGGGG | 7319 |
| hs132674616 | AGTTTTTTAGAATTCTTGCTGGG | 7320 |
| hs132675163 | TGTAATAGACACTTTGTGGATGG | 7321 |
| hs132675231 | TTACACAGGTTAAAATTGAGAGG | 7322 |
| hs132673973 | TACAGTTAATAATTTCCTGAGGG | 7323 |
| hs132674740 | GAAGGTTAGGAATTTTACGTTGG | 7324 |
| hs132673533 | TTAATAATAGTAAAAAAGCCAGG | 7325 |
| hs132672920 | GTGATTATAGAGGTGTAAGCAGG | 7326 |
| hs132673735 | CTTCAGCAATGTGATGAGTAGGG | 7327 |
| hs132674313 | AGACTATGCATGAAGTACTAAGG | 7328 |
| hs132672732 | AGTTGAGTAACTATATATGTAGG | 7329 |
| hs132674096 | ATTTGTTAATAGGTGACCATCGG | 7330 |
| hs132674060 | AGTCAGATGCCATAAATGACTGG | 7331 |
| hs132672309 | TCAGGCCAAGTTTCTGCCAAGGG | 7332 |
| hs132674961 | AGCCACACTAAAGTTATCAAAGG | 7333 |
| hs132673479 | CTACAGGTAATAACATGACTTGG | 7334 |
| hs132674432 | AATATGTCATGAATTAATAACGG | 7335 |
| hs132673119 | ATTACTAGGAGATTCAAAAACGG | 7336 |
| hs132672372 | TAAGTTCCATTTGTATTGGTAGG | 7337 |
| hs132675018 | GGTCCCTTTATACTCCTGTAGGG | 7338 |
| hs132675220 | CTTTGTCATCAAAAAGGCAATGG | 7339 |
| hs132674960 | GCCACACTAAAGTTATCAAAGGG | 7340 |
| hs132674335 | GATTTTGTTTAGAAAGAACATGG | 7341 |
| hs132672275 | TATGAAACACTCATCAGAAACGG | 7342 |
| hs132675450 | TGGGGAGATCCTCTGTTGGATGG | 7343 |
| hs132674038 | AAGAAATCCCTGGATAGGCCAGG | 7344 |
| hs132672341 | ACAAAGTGCTGGAAGTAAAGTGG | 7345 |
| hs132673292 | GTGTGTTATCTGTTACCAAAGGG | 7346 |
| hs132672403 | AAAACTCCTAAAGTCACAGTTGG | 7347 |
| hs132675102 | GTGAAGCAATAAATAGAAGAAGG | 7348 |
| hs132672956 | TGCTTTATTTGAGCTTAGAGAGG | 7349 |
| hs132673584 | GTTCCATTTCCTCCCCACATTGG | 7350 |
| hs132672951 | GATAAACTACAAAGAGTGCCTGG | 7351 |
| hs132674321 | GGAACATGTATTCCCAAGGTTGG | 7352 |
| hs132672278 | TGAACCTCATGGCGGTCTGAAGG | 7353 |
| hs132674688 | GGTAAGTGTGTGTACTATGAGGG | 7354 |
| hs132673064 | AACAGCAACAACAAAAGGGGGGG | 7355 |
| hs132674987 | ACACTTGCAAAAGTATAACTAGG | 7356 |
| hs132674168 | GTAATAGGATTACATTTACCTGG | 7357 |
| hs132672781 | CTAATTTTTTTCAGAATGGGAGG | 7358 |
| hs132673550 | AAGCTCTTGTTACTTTGAGAGGG | 7359 |
| hs132673706 | GCACAATGATTTAATGGCTAGGG | 7360 |
| hs132673679 | ACTATGAGTGGCAGTGCAGAGGG | 7361 |
| hs132674126 | TCATGCTCTAAACACCACTAAGG | 7362 |
| hs132672810 | GTTTGCTTATTCAACACCCATGG | 7363 |
| hs132673229 | CCTCCAAAAGATGAGATTCGAGG | 7364 |
| hs132674071 | CTACTCTCCTCCCTCAAAGCGGG | 7365 |
| hs132673493 | AACATATCCCAGGTTAGGAATGG | 7366 |
| hs132674289 | TTTCATATAGCCATTGAAGAGGG | 7367 |
| hs132673611 | ATAATATCATTATTGTACTCAGG | 7368 |
| hs132672966 | AAAAATACAGGAAGAAGAAGAGG | 7369 |
| hs132672858 | TGATGCCAAGAACACAACTCTGG | 7370 |
| hs132672394 | CAAAAACTATCCCAGCGAAGAGG | 7371 |
| hs132675066 | GCACTGTTAGTGTAGTTGTAAGG | 7372 |
| hs132673416 | TTCAGGGCAGTGATGCGGGGTGG | 7373 |
| hs132675377 | CTCCTATTCTGTAAGTTCAAAGG | 7374 |
| hs132675043 | ACAATGGTATTAAATATTGGTGG | 7375 |
| hs132673081 | GATTTAGTGTGAGGAAAGAAAGG | 7376 |
| hs132674158 | ATGTTTGGATTCATAAGTGTTGG | 7377 |
| hs132672201 | GTTTGCTGCTGGTTGGCTGGGGG | 7378 |
| hs132674876 | TTTAAATATACCAAAATCCACGG | 7379 |
| hs132672272 | TCACTATTTTCATTCTGAGACGG | 7380 |
| hs132674854 | CCATCCTTAGGTTTGAGCAATGG | 7381 |
| hs132673437 | TTTATAGAGCATTTGTATGGGGG | 7382 |
| hs132673137 | TAATGTGGTTGCGTAGGCACAGG | 7383 |
| hs132672252 | CATTTATGATGACTGCACTAAGG | 7384 |
| hs132672683 | CTGAACATAAATCACATGAATGG | 7385 |
| hs132674704 | TGTAAAACAATTTATTCCCAAGG | 7386 |
| hs132672842 | GGCTGTGATATACTTCACATTGG | 7387 |
| hs132672684 | ATCTAGAGAGGAGCAATAGTAGG | 7388 |
| hs132674342 | TGTGAACAATGATTTAAAGTAGG | 7389 |
| hs132674776 | CAGTATTCATGTAGGCTCCCAGG | 7390 |
| hs132673464 | CTGCTTATAGTAATGAATCATGG | 7391 |
| hs132672792 | TGACCAAAATTGGTCACAGATGG | 7392 |
| hs132672894 | TCCTACATCTAGTAAACGTTAGG | 7393 |
| hs132672148 | ATTTCAATAGCTGGAAATGAGGG | 7394 |
| hs132673556 | ACATTCTTCCTCACCTCTATGGG | 7395 |
| hs132672315 | AGCAAGATTTCAAAGATAGCAGG | 7396 |
| hs132674110 | GGTCATTTATATGATATGTCAGG | 7397 |
| hs132673421 | ATACCTACACAACAACTGTAAGG | 7398 |
| hs132672840 | ATTATGTTAACAGCTCCTCTTGG | 7399 |
| hs132674095 | TCACCTATTAACAAATAAGATGG | 7400 |
| hs132673167 | CAGTTTTGATAGAATAAATGAGG | 7401 |
| hs132672826 | GTATATTTATACATTCCAGTTGG | 7402 |
| hs132675004 | TCTGGTCTGAGGAATACTGCTGG | 7403 |
| hs132673795 | ATTTCTTCTACGAAAAGTCCAGG | 7404 |
| hs132672726 | GCCTCTTTATATCGGGGAAGAGG | 7405 |
| hs132674380 | ACTCCTTCTGTGCCCCAATACGG | 7406 |
| hs132674762 | GTGCCCATCTTGTCAATACATGG | 7407 |
| hs132672443 | GGATTTCAAAATATGCCTGATGG | 7408 |
| hs132672449 | AGGACTGCCCTTATACCATCAGG | 7409 |
| hs132674703 | ATACAATTATCAATTACCTTGGG | 7410 |
| hs132673219 | ATATATGATCCTTTTTTCTGAGG | 7411 |
| hs132674745 | GATTACTGGAGTAGAGGCAAGGG | 7412 |
| hs132673505 | AGTTAAGAAGCAGTGGTCTAGGG | 7413 |
| hs132672457 | ACAATTATGTGCCAATTGGAAGG | 7414 |
| hs132673121 | GAGTTGATACCATATAAATTGGG | 7415 |
| hs132673948 | TCAGCAATATGCGGCACACCTGG | 7416 |
| hs132673256 | AGGATTTTGTGTTTAAAAAGGGG | 7417 |
| hs132674101 | ACTGCAATATTAGGAGGCTATGG | 7418 |
| hs132672520 | TGTGTCTTACTCTACATTCAGGG | 7419 |
| hs132672717 | ATTTACACTTGAGTCAACATTGG | 7420 |
| hs132675005 | AGAATGAGTGGGAAGGTCACAGG | 7421 |
| hs132673541 | ATATCTCAAACCATAAATGATGG | 7422 |
| hs132673383 | TAGACATTGTGTTTCTTCCAGGG | 7423 |
| hs132673441 | ACAGAACAAGATCAGAAACTTGG | 7424 |
| hs132674178 | AATGAGCCTCAAAAAATACTAGG | 7425 |
| hs132672464 | GGAAGAAACGAGTGGACCATTGG | 7426 |
| hs132674327 | CCTTCAGTTACACCAACCTTGGG | 7427 |
| hs132673570 | CTTGCCACGTAGGTCCCCAATGG | 7428 |
| hs132672728 | GCCTCTTCCCCGATATAAAGAGG | 7429 |
| hs132674636 | TTCGGGACTTGAATCACAAAAGG | 7430 |
| hs132672599 | GATAAGGAGTCAGGCTCCATAGG | 7431 |
| hs132673435 | TTGTAATGTTACTCATTCATGGG | 7432 |
| hs132673227 | CTCGAATCTCATCTTTTGGAGGG | 7433 |
| hs132672334 | GTTGGCACAACCAAAACCTATGG | 7434 |
| hs132674190 | GAATAATTCATAAATGGAGATGG | 7435 |
| hs132673817 | TGGATAACTGGTGAAAGCCTTGG | 7436 |
| hs132673845 | GAGTTAAACCCTGAATTGTAGGG | 7437 |
| hs132673540 | TTAATAATACATGTAATGCATGG | 7438 |
| hs132673610 | TATCATTATTGTACTCAGGTTGG | 7439 |
| hs132672363 | AAGCATGTTGAAAAGCGTAATGG | 7440 |
| hs132674724 | AATATGTGTGAAGACCAGTTAGG | 7441 |
| hs132673652 | AAACAGAGATTTTTCCCTAACGG | 7442 |
| hs132673142 | TGGATGATGGACTTTACCTAAGG | 7443 |
| hs132675058 | AGAATCTTTGTATCATTGGATGG | 7444 |
| hs132673589 | ATCAGTCTAGTAAATTTGGTAGG | 7445 |
| hs132672051 | TGTCAATTCAAATGGTGCACTGG | 7446 |
| hs132672337 | AGGTTATTGTTTACAACCATAGG | 7447 |
| hs132674384 | TCAAACTACTATTATATCAAGGG | 7448 |
| hs132674872 | GCAGAGTAAGAACTACTGTATGG | 7449 |
| hs132672303 | TGAAGGTTACACCCAGCGACTGG | 7450 |
| hs132672815 | CAGTCAACCTTCTATTTCCACGG | 7451 |
| hs132673245 | AAGAATCATGACTCTTTGAGAGG | 7452 |
| hs132672580 | CTCTCAAATACCTCTCCCTCAGG | 7453 |
| hs132674589 | AAGTTATGCTTCCTTCAGATGGG | 7454 |
| hs132673908 | ACATACAGATGAAGTAAAAGAGG | 7455 |
| hs132673972 | TTAACTGTAAAATAAGATAGAGG | 7456 |
| hs132675409 | AGCTAAAAACAGGGGTAGGAGGG | 7457 |
| hs132674827 | AATCATTTTTAAATAGCCCCAGG | 7458 |
| hs132674525 | TCAAGTAATGGGACTAAGACCGG | 7459 |
| hs132672563 | GTTGCTAATATCTAGTCAAAGGG | 7460 |
| hs132674645 | GCCTGACAAACTGCCCTCAATGG | 7461 |
| hs132675256 | GAATCCGTGTCACCATTCTCAGG | 7462 |
| hs132673559 | AACTGGACTGTTTAATGTGATGG | 7463 |
| hs132673956 | TTATTGCCATAGAGAAATTGAGG | 7464 |
| hs132673978 | CTATTCCACTGCTATGTATGTGG | 7465 |
| hs132672484 | AATGCGTGATGTTCGTCCACTGG | 7466 |
| hs132672261 | TGAGTACTATAAAGTATTTGAGG | 7467 |
| hs132673644 | TGATTGTGAGCCATTTCCATTGG | 7468 |
| hs132674002 | AGTAGGGCAGATATTTGCCCTGG | 7469 |
| hs132673642 | ATTTGTTCATATTAAGACATAGG | 7470 |
| hs132674041 | GTGCTTCTTGAAGAAATCCCTGG | 7471 |
| hs132672504 | TGTGAAGCATTGATGGAGAAAGG | 7472 |
| hs132674475 | GATTTTATGTTAGCCATCACGGG | 7473 |
| hs132672622 | TTGGGGCCAAAACCGTCAATGGG | 7474 |
| hs132674341 | AATTAGTGTTTGAAGATGACAGG | 7475 |
| hs132674689 | GGGTAAGTGTGTGTACTATGAGG | 7476 |
| hs132674226 | GATGCTGTCTTTAGAATTTGGGG | 7477 |
| hs132673553 | TAAGGAAGTGACTTGCTGCCTGG | 7478 |
| hs132675069 | GATATGCAACTTTTACTGACAGG | 7479 |
| hs132673945 | GATTAAGCTAGGAAAGAAAGTGG | 7480 |
| hs132672103 | TTTTCTAGAATTTCAAGATACGG | 7481 |
| hs132672387 | CCATAAAAGCCCTCTTCGCTGGG | 7482 |
| hs132672974 | AAGTGGCTATGGCCAACGTCAGG | 7483 |
| hs132673055 | TCAAGTTTTAATTTTCCCACTGG | 7484 |
| hs132674763 | ACACATACATCCGCATACTAAGG | 7485 |
| hs132675305 | CTTTTAAAATGAGTAGCAGTAGG | 7486 |
| hs132672770 | GGTGATAAACGTTCAGAGGTTGG | 7487 |
| hs132674300 | CTGTGTCACCTCAAATTCTGTGG | 7488 |
| hs132672206 | AGGGAGTGTTTGCTGCTGGTTGG | 7489 |
| hs132672422 | TCTCTCGACATGCCTGTTGTGGG | 7490 |
| hs132672774 | GCACGTGTGCACATGCAAGCTGG | 7491 |
| hs132674257 | ATTTTCCTCTTATTGACTTGAGG | 7492 |
| hs132673278 | GCACTGAACAGGTCATTAGTTGG | 7493 |
| hs132674066 | ACAGTAATGTGCCCGCTTTGAGG | 7494 |
| hs132673502 | GGATCTGTGTGAGATTCTGCTGG | 7495 |
| hs132673554 | GAGAGCTGTGAACAATTGTAAGG | 7496 |
| hs132675298 | TCTTACAGAGAAATCATAACGGG | 7497 |
| hs132672311 | GTTTCTGCCAAGGGACAGGCAGG | 7498 |
| hs132673653 | ATATACATGATTTTCCGTTAGGG | 7499 |
| hs132675081 | TAATAACTAAGGGCTAATAAGGG | 7500 |
| hs132675253 | CAAGAATTAGCTCTGCCTGTTGG | 7501 |
| hs132673578 | TTTGGGTTCTGTATGTACCTAGG | 7502 |
| hs132672293 | CCTTCAACAAAACTAATCTTGGG | 7503 |
| hs132673358 | AAAACTCTGCTTTCCTTCCACGG | 7504 |
| hs132672300 | GTGTAACCTTCAAACACCGCTGG | 7505 |
| hs132673316 | CAAGATGCCTCATAGGTAAGAGG | 7506 |
| hs132673468 | ATATAGTCGAAGACTTTTCAGGG | 7507 |
| hs132672764 | CCCAGAATACCTCAAGAGCTTGG | 7508 |
| hs132672581 | TCAGGTAGAGCTCTACTAGCAGG | 7509 |
| hs132673651 | TATATATGTAGAAATGTACTAGG | 7510 |
| hs132673861 | TTCCTGTTTCTGCTGTTAGAGGG | 7511 |
| hs132674398 | TTTAGTTTGAGCATGTCACTTGG | 7512 |
| hs132674232 | ATACAATCTTTGCCCACTCGTGG | 7513 |
| hs132672469 | ATGCCATCTGGTTTGTCCAATGG | 7514 |
| hs132672501 | TGGAAATTACTTGACATCCATGG | 7515 |
| hs132674203 | TCAGACTGCCTCTAAAATAGAGG | 7516 |
| hs132674378 | TTAGTTTGCTAGGTTTGTAGTGG | 7517 |
| hs132672825 | AGTTGGACCAATTTTTTGAAAGG | 7518 |
| hs132674450 | CTTGTAAACGTAGAAAGAAAAGG | 7519 |
| hs132673604 | TCATTGCATACCATTTAGCTGGG | 7520 |
| hs132674906 | CTTTCCTCAGATTGATCAAGAGG | 7521 |
| hs132675130 | AAAAAGAGAATAAATCTAAGAGG | 7522 |
| hs132672256 | TTAGGTGATCACAGAGTGCCAGG | 7523 |
| hs132672102 | TCTACTATATAAGAAACAATTGG | 7524 |
| hs132673522 | AGGGAAGGTGGTGGTTAAAACGG | 7525 |
| hs132674130 | TATAGATTGGACAATAAGGTTGG | 7526 |
| hs132673411 | TCATTGGCTCATTATCTTCAGGG | 7527 |
| hs132672882 | TAGAAATTGCATATATTCCAGGG | 7528 |
| hs132674943 | TTTTCTGTATAGAATCAGTTGGG | 7529 |
| hs132672709 | GGCTTCCTGACTTTAAGAAAAGG | 7530 |
| hs132674861 | CTAAACCTTACTCAAAATAGTGG | 7531 |
| hs132674986 | GTCATATGACATCGTAAAAGTGG | 7532 |
| hs132673815 | GCTGAAGTAGTGATTGTCCTTGG | 7533 |
| hs132673028 | AAGTAGACTTAGAGAGCCATAGG | 7534 |
| hs132672535 | CCAAATTAGAGAGCATTAGCAGG | 7535 |
| hs132672849 | GATGAAGATTTTAAGTGCCATGG | 7536 |
| hs132673391 | ATAGAAACTATGCTAGGCACAGG | 7537 |
| hs132672783 | AATTTTTTTCAGAATGGGAGGGG | 7538 |
| hs132672989 | ATGAATCAGTGTTTGAAAAGAGG | 7539 |
| hs132675031 | GTTTCTATGTTCATTTTCCTAGG | 7540 |
| hs132673531 | TAGCTATGCCTTCTATATGTAGG | 7541 |
| hs132674373 | ACATCTAAACATTTTTTGGAAGG | 7542 |
| hs132672671 | GCTAATAAAGCCTTCAACTGTGG | 7543 |
| hs132672897 | TTTGGTGTCCTTTATCGTCATGG | 7544 |
| hs132673637 | ATCAATAGTGAGAATTTATGTGG | 7545 |
| hs132672855 | TCTTTAGCATTTTTAAACAATGG | 7546 |
| hs132675361 | GGAAGAGTCTATATATAGAAAGG | 7547 |
| hs132674907 | ATAGCAAAGGTAGTGCTTGAAGG | 7548 |
| hs132674414 | TAGAAGGAAACACCTGAAACCGG | 7549 |
| hs132673944 | ATTAAGCTAGGAAAGAAAGTGGG | 7550 |
| hs132674482 | AGTAGAGCTGACTGAAAGCCTGG | 7551 |
| hs132673037 | AAGAGATAAGACTAAAAGAAAGG | 7552 |
| hs132672572 | TTTTCTCAGTGGCTGGATGAGGG | 7553 |
| hs132675098 | GTTTCCAGCAAGGGTTGCCCAGG | 7554 |
| hs132675181 | CTTTAACAAATCAGCCCCTAGGG | 7555 |
| hs132672674 | CTAAATTTACCTAAATAAAAGGG | 7556 |
| hs132674557 | GCAGCATTATCAATTTATCCAGG | 7557 |
| hs132675079 | ATCCACTTGAAATAAGCAAATGG | 7558 |
| hs132674039 | CAAGAAGCACAGCTACTGTATGG | 7559 |
| hs132674674 | CGCACATTCCCTTTTTCATGAGG | 7560 |
| hs132672048 | TAATATAACTTTCCATTGGAGGG | 7561 |
| hs132675299 | AGAATTGTCTGATTCACAAAAGG | 7562 |
| hs132672344 | CTAAGTTGTAGACCATATCATGG | 7563 |
| hs132675395 | TTAGTTCAGTGAGGTAGAAAGGG | 7564 |
| hs132675210 | CTTCTAACTTTTAGGCCACTGGG | 7565 |
| hs132674447 | AGAAAGAAAAGGTTAGGAAAGGG | 7566 |
| hs132674641 | TGCTGGACTTCTACCATTGAGGG | 7567 |
| hs132672758 | ATAACAAAATTTAATTCTGGTGG | 7568 |
| hs132673376 | AATAAAATAGTATTGAGTGATGG | 7569 |
| hs132674437 | CCAAAGGAGATTCATCTTGTGGG | 7570 |
| hs132674403 | ACATTATTCTTCTCAAGGATGGG | 7571 |
| hs132673804 | CATCATATCAAGTTGCCCATAGG | 7572 |
| hs132672597 | AGATAGTTATACCAAGCCCCTGG | 7573 |
| hs132674055 | TGATTATCTTTAAATGTGACAGG | 7574 |
| hs132674025 | CGAGTGTGCATTTATATATGAGG | 7575 |
| hs132672095 | ACAAAGTTATACAACACATATGG | 7576 |
| hs132674630 | TATCCTCTACGCTTCTTACCTGG | 7577 |
| hs132672529 | CTCTTTTGCAGAATTTTGTAGGG | 7578 |
| hs132675185 | TTATAATTTCTATCTCCCCTAGG | 7579 |
| hs132674207 | TAATGTATTTTTGAAAGCATCGG | 7580 |
| hs132672573 | CTCATCCAGCCACTGAGAAAAGG | 7581 |
| hs132674133 | ATTGATTCTGAAAGATATCAAGG | 7582 |
| hs132674500 | TATTTCTTCTGTTCCAGGATGGG | 7583 |
| hs132675117 | CTGGATCTTATCCTATAAAAGGG | 7584 |
| hs132675468 | TTTAAAATGTGTATGCCCTATGG | 7585 |
| hs132674280 | CCCTTCACTTTCCACATGAAGGG | 7586 |
| hs132674496 | GCCCAGAGCAGAAATTCAAATGG | 7587 |
| hs132674883 | AGGTATTGAAAATTGCATATAGG | 7588 |
| hs132672232 | TTTACAGTTTTACTAAACATTGG | 7589 |
| hs132673303 | ACTGGGATGTGAATGCCAATAGG | 7590 |
| hs132672618 | GAGAAAACACATCCCATTGACGG | 7591 |
| hs132673163 | ATAGAAATGAATCTTTCAAAAGG | 7592 |
| hs132672793 | ATTTTGGTCAATTTTTTCACAGG | 7593 |
| hs132673805 | TCTAGTTTATAAAAGTCATCAGG | 7594 |
| hs132672576 | GGAAGCTATACCAAGAAAATGGG | 7595 |
| hs132674978 | ATTCTGTAGTAGTTACAAGTAGG | 7596 |
| hs132673796 | GCTAAATGTGAATGTCTAGTAGG | 7597 |
| hs132674939 | CCTCAAGAGGCAAGAGTACATGG | 7598 |
| hs132674968 | AAACATTTGGAATCAACTGATGG | 7599 |
| hs132675184 | TATAATTTCTATCTCCCCTAGGG | 7600 |
| hs132675119 | ATTTGCACTCTTCTACCAAAAGG | 7601 |
| hs132674311 | AGATGACTATTCCATGAAGCTGG | 7602 |
| hs132672133 | AATTACTTATTTCTACCTACTGG | 7603 |
| hs132673275 | GTTGGGATTGAGTCAAGAGTTGG | 7604 |
| hs132674882 | ATATGCAATTTTCAATACCTTGG | 7605 |
| hs132673740 | TTTTCCTTTATAACATAATGAGG | 7606 |
| hs132673625 | ACACGTTTGGACATTATTTGAGG | 7607 |
| hs132675158 | CTTGTCACAGAATTTTGCCTTGG | 7608 |
| hs132672808 | AATTTATTATCTTGGTCCATGGG | 7609 |
| hs132674714 | TTGTTGTCTAAGCTGAAGAAAGG | 7610 |
| hs132674293 | ATATTTTTCTGCCTTTGTTGTGG | 7611 |
| hs132674544 | TGCACAAGCATATTTTTGGTTGG | 7612 |
| hs132674892 | TCAGACTAAGAATAAAAAGTAGG | 7613 |
| hs132672913 | AACTGAATTTTTGTACCAGTTGG | 7614 |
| hs132672950 | CTTTGTAGTTTATCAAGAGCAGG | 7615 |
| hs132675249 | CAGGAGTAAAACTTTCCAACAGG | 7616 |
| hs132674455 | GTATCCTAAAGAGTTAATATAGG | 7617 |
| hs132672142 | TTAAAATCATATAATATTGGAGG | 7618 |
| hs132674932 | TGTTGTAGTTGTTAATCAAAGGG | 7619 |
| hs132673535 | ATATTTTGAAATGAGTCTTAGGG | 7620 |
| hs132675416 | CTTCTCCTTTGGATAATGCATGG | 7621 |
| hs132673909 | TAGAGAAGAGGGATGGGCATAGG | 7622 |
| hs132675086 | ACATTTTTATTGGCTGCAAAAGG | 7623 |
| hs132673772 | GAAAATGGTTGTGTATCAAAAGG | 7624 |
| hs132674413 | ATTACTCCTTGAATTGGCTATGG | 7625 |
| hs132672969 | AAGATAAGAATAAATCCAACAGG | 7626 |
| hs132673810 | ACAGATCTACACATAGATAGAGG | 7627 |
| hs132672688 | ATAAATTCTATTTATTGCTAGGG | 7628 |
| hs132672741 | ATAATTAATATAAGATAGACTGG | 7629 |
| hs132675267 | TGACATTTACAAACCCGTTAGGG | 7630 |
| hs132672082 | TTAAGCTAAGATAAATTGTATGG | 7631 |
| hs132673875 | TGACCCATTATTTTATCAAGTGG | 7632 |
| hs132674003 | TTCTCTGTGGCAACAAAGTAGGG | 7633 |
| hs132674521 | TGGAACCCTTGCCCTTTAGCAGG | 7634 |
| hs132675373 | TTTTTTGTGGATATATACATAGG | 7635 |
| hs132674514 | AGGGATGTCTGAAATTCCTTAGG | 7636 |
| hs132672196 | GGGTAGATTGAAACTTTTGTTGG | 7637 |
| hs132674520 | TTAGCAGGCCATTTCTCCTAAGG | 7638 |
| hs132674555 | CGAGCACTGATAAGATTAGCTGG | 7639 |
| hs132674730 | ACTACCTACAGCATTGTGATAGG | 7640 |
| hs132674261 | CTATCTGGTGGTCTTCTCAATGG | 7641 |
| hs132673368 | CTGTGGTTCCCCCAAGATAAGGG | 7642 |
| hs132675240 | ATGTTGGGTTTGATATCAATTGG | 7643 |
| hs132672080 | CATTACCTTGACCTCCTGCCAGG | 7644 |
| hs132674532 | ACGAATTGTTTCTTTCCCTCCGG | 7645 |
| hs132675217 | AGCAATTTCCTCTTCCCTGATGG | 7646 |
| hs132672695 | TCAATGTACAAACTAACTTAGGG | 7647 |
| hs132672816 | GTTGCATCCGTGGAAATAGAAGG | 7648 |
| hs132672445 | AAATATGCCTGATGGTATAAGGG | 7649 |
| hs132675068 | TGACAGGACATAATGAACTTTGG | 7650 |
| hs132674132 | TAGACACTTGAAATATAGATTGG | 7651 |
| hs132673924 | TTACACTGAAAGTTGCTAGAAGG | 7652 |
| hs132674162 | CTTGAATTGAATCAGAGAAATGG | 7653 |
| hs132673287 | CAGCTACCACCCATTCCCTTTGG | 7654 |
| hs132674379 | AGAAATCTACTTAGTTTGCTAGG | 7655 |
| hs132672400 | ATTTGCTCTGAGGCACTCTCAGG | 7656 |
| hs132673333 | TGTCCAGATGAGCTTTAGCTTGG | 7657 |
| hs132673451 | GCAAAGGTAAAAATAATTGTAGG | 7658 |
| hs132674675 | TTTAAAAACCTCATGAAAAAGGG | 7659 |
| hs132672508 | ATCATCATATTTGTGTTACATGG | 7660 |
| hs132674016 | TCAGGTTTACTTGCAAGGAATGG | 7661 |
| hs132674796 | ATTTTTGTGGGTACATACTAGGG | 7662 |
| hs132674653 | ATACCAGAATTAGAGTATATCGG | 7663 |
| hs132673347 | ACCAGAAAGAGTACAAATAAGGG | 7664 |
| hs132673030 | AGAGTGTCTTCCTAGGCAATAGG | 7665 |
| hs132673123 | GCAAAAGCCTATAGACATACAGG | 7666 |
| hs132674361 | TCTAACCTAAATGTATTTGAGGG | 7667 |
| hs132673155 | TCCAATTCTGTTCTGCAGACGGG | 7668 |
| hs132673536 | TTGAAATGAGTCTTAGGGCTAGG | 7669 |
| hs132672742 | TTAATATAAGATAGACTGGTTGG | 7670 |
| hs132673848 | TTAACAAGTTATATGATTCAGGG | 7671 |
| hs132674258 | AATTACCTCAAGTCAATAAGAGG | 7672 |
| hs132672433 | TATGTTAGCGAGGAAGATAAGGG | 7673 |
| hs132675293 | TCTCTGAGTTTCAAACAGATTGG | 7674 |
| hs132674742 | TCATATATAACTCAATATACCGG | 7675 |
| hs132675443 | TTGCCATGCCAATTTTGAAATGG | 7676 |
| hs132673011 | GCTCTATATGACCATGGTCCAGG | 7677 |
| hs132674458 | GTTAATATAGGATGTAATATAGG | 7678 |
| hs132673230 | GTCTGTACCTACCCAGATATAGG | 7679 |
| hs132672701 | GATTAGTAAGAACCTTTCTCAGG | 7680 |
| hs132672071 | GTAGCAAATGAATCTTTTGAAGG | 7681 |
| hs132673608 | TGGCTGTTAAAAAGTCATAAAGG | 7682 |
| hs132674405 | GTATTTTTATCTTTTTGACTTGG | 7683 |
| hs132672258 | GAGTTTCTTAACAAATACTTAGG | 7684 |
| hs132672517 | AATGAACCTTTATTCCCAAAAGG | 7685 |
| hs132674517 | AGGAGAAATGGCCTGCTAAAGGG | 7686 |
| hs132674215 | GAGATTTACAGACACATAGTAGG | 7687 |
| hs132674206 | AGAAAATTTTGTAATGAGGTGGG | 7688 |
| hs132673630 | AGCAAATCTTGGCTTTAGCCAGG | 7689 |
| hs132673127 | CTAGAGCTCAAGTGAAATATGGG | 7690 |
| hs132672868 | GTAAAGACTCTAATATATAAGGG | 7691 |
| hs132672416 | CACTAACTTGACATAACAAATGG | 7692 |
| hs132673723 | CCCTTCAAAGCCTTTCCTTACGG | 7693 |
| hs132674914 | TAAAAAATATGGAATATTAAGGG | 7694 |
| hs132674021 | CCTTTTGATCTAGCTATCCTGGG | 7695 |
| hs132674859 | ACTTTTGCTTATGTTAGCTCAGG | 7696 |
| hs132674426 | TTACCATTTCAAATTATGAATGG | 7697 |
| hs132673646 | GCGTCTGTTTTTTCATCCAATGG | 7698 |
| hs132675413 | GTGTATAGAAGCAATAAAAAGGG | 7699 |
| hs132673880 | TTAACTGTCAGTGGTCATAAAGG | 7700 |
| hs132675447 | ATAACTTTGACTCTTATCTCTGG | 7701 |
| hs132673332 | GTCCAGATGAGCTTTAGCTTGGG | 7702 |
| hs132673042 | GGAATCTTTAATATAGGTAGTGG | 7703 |
| hs132673745 | GCTCAACTTTTTTTTTGTATGGG | 7704 |
| hs132674953 | GTTAACTGAGAAAGAGTAGAGGG | 7705 |
| hs132672968 | CTTCAGTGAGAACTTCCTGTTGG | 7706 |
| hs132672672 | CATGTCTCTAGAGATAGAATAGG | 7707 |
| hs132674810 | GTGCAGACATGGTTGATTCATGG | 7708 |
| hs132675341 | CAATTCTTGCCATTGTGTTAGGG | 7709 |
| hs132672092 | AAATGAATAAAACAATAAAACGG | 7710 |
| hs132673993 | GGAGACATAAGTATGTGAAATGG | 7711 |
| hs132672546 | TGCTAAGCGTCTGCATACCTGGG | 7712 |
| hs132675386 | AGAGCATACACCCCAATAATGGG | 7713 |
| hs132674452 | ATAGAGGTAGACGTTTTAAAGGG | 7714 |
| hs132672604 | CTGATCTTGGATTGACCAGATGG | 7715 |
| hs132674153 | AACCTCAACTTAAACCCAAATGG | 7716 |
| hs132673508 | TTTGCGCAACAATTTTCAAATGG | 7717 |
| hs132673532 | ATTCAAAGCCTACATATAGAAGG | 7718 |
| hs132672110 | TGTATAACACAGCAGTGAATAGG | 7719 |
| hs132672047 | TATTAACATGTCAATTCAAATGG | 7720 |
| hs132672302 | ACACCCAGCGACTGGCTAATCGG | 7721 |
| hs132674263 | AAGCTGATAGTTCCTAACTATGG | 7722 |
| hs132674731 | GTGATAGGCCAGCAGTACTATGG | 7723 |
| hs132675308 | GCCTGAGGTTTAGTTTTAAGTGG | 7724 |
| hs132672162 | AGTGAAAACTAAGATGAAACTGG | 7725 |
| hs132673061 | ACCAACAGCAACAACAAAAGGGG | 7726 |
| hs132673882 | TTATGACCACTGACAGTTAAGGG | 7727 |
| hs132674727 | AGGAAGTCACCACCAATAACAGG | 7728 |
| hs132674419 | AGTTCTATCTTCTTCTGTACCGG | 7729 |
| hs132675113 | CTAGAAGTCTAAACAAATGCTGG | 7730 |
| hs132673906 | TTACATGTCTGTTTTGTTTGTGG | 7731 |
| hs132672738 | ATAAGTTGAGATATACTTTGAGG | 7732 |
| hs132673169 | AGGCAGAAGACAGCTTTTAAGGG | 7733 |
| hs132673562 | AACAGAACCTTTTCTCCCATTGG | 7734 |
| hs132672753 | GTTTAAACTTTGCAATACTAAGG | 7735 |
| hs132674963 | CATTAAATAATTAAAGTAGAAGG | 7736 |
| hs132674603 | TTAAGGAAAGAGCTATCTTAAGG | 7737 |
| hs132672740 | TTTCAATTCATCAGATTATGTGG | 7738 |
| hs132673009 | TTATTACATAGTCAGTACTATGG | 7739 |
| hs132674388 | GAAAGATGCATAACAACAATTGG | 7740 |
| hs132673992 | CACTACCTTCTATTAAAAAGAGG | 7741 |
| hs132672992 | TTAAAGATAGATTGGGGGGGTGG | 7742 |
| hs132673558 | TCACCTCTATGGGTACTAACTGG | 7743 |
| hs132673761 | ACAGGAATATTTGCTCTTTGAGG | 7744 |
| hs132674510 | TGTGCGAGTGTATCTTTCTAGGG | 7745 |
| hs132672217 | AATCTAAGAACATTTCAGATTGG | 7746 |
| hs132674825 | GAACTCATCTGTGTGTTTAGAGG | 7747 |
| hs132675047 | CCGTTGTCCTAGCAGCTATATGG | 7748 |
| hs132674367 | ATATTGCATGCGCTAATCATAGG | 7749 |
| hs132674599 | TTACATGTCTAAATACCAATTGG | 7750 |
| hs132673161 | GATATGGATATACTTATAGATGG | 7751 |
| hs132675264 | GATTAAAACCAAACCCTAACGGG | 7752 |
| hs132674541 | ACATCAGTGGTTTAGTTTTGAGG | 7753 |
| hs132673482 | TGGTCCTGTTCTCTGGGTAATGG | 7754 |
| hs132672537 | ACTTTGTCATCATCACTCATAGG | 7755 |
| hs132672623 | CTTGGGGCCAAAACCGTCAATGG | 7756 |
| hs132674691 | AAGCAATACACACTACATATCGG | 7757 |
| hs132673331 | GGACAATCACAAGGGTTTAGAGG | 7758 |
| hs132672470 | CATCAGTGTTGTATGCCATCTGG | 7759 |
| hs132672404 | TGCCGTGTGCCCTAATCAACAGG | 7760 |
| hs132674989 | ACTATCATGAACCAGCCTTCAGG | 7761 |
| hs132672658 | GTCATTCTGCAGTCCATCATAGG | 7762 |
| hs132674667 | GCAGTAAAATCAATAATAAAGGG | 7763 |
| hs132675428 | GATTTCATCAATTCCAAAACAGG | 7764 |
| hs132674412 | CCAATTCAAGGAGTAATAGAAGG | 7765 |
| hs132672188 | TAGCACATAACTTTGTATATAGG | 7766 |
| hs132675335 | GCATGAATCACGAATTTTGCTGG | 7767 |
| hs132675017 | TTTCCCTACAGGAGTATAAAGGG | 7768 |
| hs132674916 | GTATTTCACATGCAGTTTGTTGG | 7769 |
| hs132672458 | AATTACAATTATGTGCCAATTGG | 7770 |
| hs132672314 | TTCCATTCCTGCCTGTCCCTTGG | 7771 |
| hs132673226 | CCTCGAATCTCATCTTTTGGAGG | 7772 |
| hs132672335 | TTTTGGTTGTGCCAACATATAGG | 7773 |
| hs132672477 | CATACGCAACATCAGAATAAGGG | 7774 |
| hs132672763 | ATAACTGCACACCAAGCATTTGG | 7775 |
| hs132673663 | TGATTTACTTCTAAAATAAGTGG | 7776 |
| hs132673748 | AGGAAAAAAAACACAAAAAACGG | 7777 |
| hs132675257 | CACGGATTCTATTTTCTGCTTGG | 7778 |
| hs132672255 | GATCACAGAGTGCCAGGAATTGG | 7779 |
| hs132675262 | TTATTTTGACTTCCTGAGAATGG | 7780 |
| hs132673843 | AAGAAATTCCCTACAATTCAGGG | 7781 |
| hs132674354 | ATTCCTCTAGTATTGCTATTCGG | 7782 |
| hs132672663 | TTTTGTGTTGGTAAATTTGTCGG | 7783 |
| hs132674503 | GGAAAAACACATTAAATGATAGG | 7784 |
| hs132673040 | TAGTTACTAATATTTTTCATAGG | 7785 |
| hs132673962 | ATGAATTTCATACTGACTACTGG | 7786 |
| hs132673897 | CCAGAACAATGAGACCAAATAGG | 7787 |
| hs132672455 | GGCACATAATTGTAATTCATAGG | 7788 |
| hs132674856 | GGTGTCCCCCCACCATCCTTAGG | 7789 |
| hs132674425 | TGGCCATTCATAATTTGAAATGG | 7790 |
| hs132675161 | AATATATTAAAAACATCTATTGG | 7791 |
| hs132675277 | TTTCATATCCTAAATGGAAATGG | 7792 |
| hs132673563 | ACAGAACCTTTTCTCCCATTGGG | 7793 |
| hs132672528 | TCTGCAAAAGAGATTCAGTTTGG | 7794 |
| hs132672954 | AATGACTTCAGCCTGCTTTGTGG | 7795 |
| hs132675368 | TGAAGTTTGAGACAGTAAAATGG | 7796 |
| hs132673573 | GTATGTACCTAGGACCTAATGGG | 7797 |
| hs132672598 | TCATAGCTCCAGTCCCCATATGG | 7798 |
| hs132672984 | TTAATGTAGCACTTCAGCTTTGG | 7799 |
| hs132673614 | AGAGAGCAGGGGAAGAAAACAGG | 7800 |
| hs132674936 | CAGTGAGGTGAATTTATTAAGGG | 7801 |
| hs132675314 | GGAAGTCATTGAAACCTTGTAGG | 7802 |
| hs132673873 | CATCCACTTGATAAAATAATGGG | 7803 |
| hs132674720 | GCTTGTTAATGTTAATTCCTTGG | 7804 |
| hs132674286 | ATTTGCTTCCCCCTCTTCAATGG | 7805 |
| hs132674838 | CGTTTCTATAGCTCTCAAAAAGG | 7806 |
| hs132672544 | GCTATGCTTCCAAAGTTCTAAGG | 7807 |
| hs132674679 | TTACTATTCAAGATATTCTTGGG | 7808 |
| hs132672785 | TAGAAGACAATGCATTTTAACGG | 7809 |
| hs132673483 | GTAATGGAGTAAAATGTTACTGG | 7810 |
| hs132672790 | TTTTTTCACAGGCATTTAATGGG | 7811 |
| hs132673187 | TGGAAAGGTGTATACTACTATGG | 7812 |
| hs132675137 | TGTCATTTAAATCTTATAAGAGG | 7813 |
| hs132672961 | TAACTATCACATTTTCTCATAGG | 7814 |
| hs132674956 | CAGTTAACTCTAAAATTAGATGG | 7815 |
| hs132675259 | TCTCAGGAAGTCAAAATAAAGGG | 7816 |
| hs132672687 | GATAAATTCTATTTATTGCTAGG | 7817 |
| hs132674642 | CAGCAGTGCTCATTTCTATAGGG | 7818 |
| hs132674440 | ATATTAGTAGTTGGGCCAATAGG | 7819 |
| hs132673538 | GCATTACATGTATTATTAAAGGG | 7820 |
| hs132673440 | AGTTTTATAGAGCATTTGTATGG | 7821 |
| hs132672454 | TTTCCTGATTTCCTTCCAATTGG | 7822 |
| hs132673645 | GTTTTTTCATCCAATGGAAATGG | 7823 |
| hs132673041 | GAGTTTGGAATCTTTAATATAGG | 7824 |
| hs132673574 | TGTATGTACCTAGGACCTAATGG | 7825 |
| hs132674018 | CATATATTGATGTTGCATATAGG | 7826 |
| hs132674180 | CAAAAAAGTAGCCATTTAAAGGG | 7827 |
| hs132674353 | TTAAATTCTACATTCTCTGCTGG | 7828 |
| hs132674166 | CGCATTATAGGCATAGTAATAGG | 7829 |
| hs132674408 | AGTGCTCTATATAAATATATTGG | 7830 |
| hs132673990 | TTTATTATCTAATTGATCCTTGG | 7831 |
| hs132675404 | TCTCCCTTAGAGCTAAAAACAGG | 7832 |
| hs132672313 | CCTGTCCCTTGGCAGAAACTTGG | 7833 |
| hs132672694 | TTCAATGTACAAACTAACTTAGG | 7834 |
| hs132674552 | GGCCTCACGCCCTATAAAACTGG | 7835 |
| hs132675300 | TGTTTATGCTCATAGATAATAGG | 7836 |
| hs132673125 | ACAGCTTCCTGTATGTCTATAGG | 7837 |
| hs132675138 | ACTATTTTACAACAATATTTGGG | 7838 |
| hs132673309 | CTTGTTCTCATGGAATCCTTAGG | 7839 |
| hs132672461 | ATAAAACGTGATTAATTGCTTGG | 7840 |
| hs132675014 | CTAGAGGCAGGCAAAATAACTGG | 7841 |
| hs132672675 | GTAAATTTAGTACACAATTAGGG | 7842 |
| hs132674332 | ACATGGTGGGAAGCAATAAAAGG | 7843 |
| hs132675448 | TTTCTGCTTCTTTAGTTGATAGG | 7844 |
| hs132672982 | GTGCCTCAAGACTAAATATTGGG | 7845 |
| hs132672468 | GGTCCACTCGTTTCTTCCTTAGG | 7846 |
| hs132674338 | TGTTCTTTCTAAACAAAATCCGG | 7847 |
| hs132673395 | ATAGTTTCTATATATTTATTAGG | 7848 |
| hs132673654 | AATATACATGATTTTCCGTTAGG | 7849 |
| hs132673600 | ATTCCTGCCAAATTATTTCATGG | 7850 |
| hs132672368 | TAGAGCATAACAGGGAAAAAAGG | 7851 |
| hs132673474 | ATTTGCAGATTTGGTGAAAATGG | 7852 |
| hs132673492 | TTACTCAAAGACGTTCTATCTGG | 7853 |
| hs132674865 | AAGGAACTATAGAAAAAGTTAGG | 7854 |
| hs132675219 | ATATTACTTTGTCATCAAAAAGG | 7855 |
| hs132674871 | ATGTCCAACTAATAAAATTAGGG | 7856 |
| hs132675456 | TCTGTTGTAATTTTTCACTCTGG | 7857 |
| hs132674050 | AGACTCAGTCCCCAGAAAAATGG | 7858 |
| hs132673378 | TATTTAGTTGTATAAGTAATGGG | 7859 |
| hs132672655 | ATACTTTGTATATTCATTGTAGG | 7860 |
| hs132673977 | GTATAATATAAACACAAAATAGG | 7861 |
| hs132672854 | TTACTGATGTAATTTATAACAGG | 7862 |
| hs132675023 | CTAAAGTTATGTCAAATAACTGG | 7863 |
| hs132672880 | GAATATATGCAATTTCTAATAGG | 7864 |
| hs132673247 | GCAACAGCGCTGACATTTCTAGG | 7865 |
| hs132674873 | GCTTCCCTAATTTTATTAGTTGG | 7866 |
| hs132673602 | ATTCTGTCTCCCCAGCTAAATGG | 7867 |
| hs132673686 | CTCACTCTCCCAAGTCAATATGG | 7868 |
| hs132672707 | TTACTTTTGCTTTTACTGATAGG | 7869 |
| hs132674715 | GTAAGATCAAGGAAAATATTAGG | 7870 |
| hs132674593 | CGCAGTTTTCCAAAAGAAATCGG | 7871 |
| hs132672828 | CTAAACGAGGTGAATAAAATAGG | 7872 |
| hs132672666 | AGAATATTACAATAATATTAAGG | 7873 |
| hs132674755 | GTATGTCTGGGGTAGAAATTTGG | 7874 |
| hs132675474 | GACTACCACTGCTTTTAGTATGG | 7875 |
| hs132672489 | TATCTTTGTAATTTGAAAATGGG | 7876 |
| hs132675075 | CTGATATTAATGCCATAAATAGG | 7877 |
| hs132674661 | TATTTCATCATTCCACTGATAGG | 7878 |
| hs132673619 | TTAGAATTTTCCTAAGGAAATGG | 7879 |
| hs132672659 | TAGGTTTTATTACTGTTTAATGG | 7880 |
| hs132674220 | GTCAAAGTCAATTTACATAATGG | 7881 |
| hs132673060 | CACCAACAGCAACAACAAAAGGG | 7882 |
| hs132672731 | ACTAGAGAAGGTAGGCAAATAGG | 7883 |
| hs132674308 | TGAAATATAAACCAGCTTCATGG | 7884 |
| hs132672570 | TCTTTCCTTTTCTCAGTGGCTGG | 7885 |
| hs132674524 | TACCCCTTCTTTCAAGTAATGGG | 7886 |
| hs132672821 | TCAAATATAATACATTTAAATGG | 7887 |
| hs132675016 | TTTTCCCTACAGGAGTATAAAGG | 7888 |
| hs132674660 | AATTTAGTCAAATTTATTACAGG | 7889 |
| hs132675222 | GCACTTACATGAATGAAAAATGG | 7890 |
| hs132675155 | GATGGTAAATGAGAATAAAATGG | 7891 |
| hs132675225 | AGGTGTTCCTTCTCTATATACGG | 7892 |
| hs132673953 | GCCTTACCTCAATTTCTCTATGG | 7893 |
| hs132672690 | CTATATTTGTATTATTTTAGAGG | 7894 |
| hs132672648 | ACAAATATCTTCTTATTATGAGG | 7895 |
| hs132673397 | GTCCTCATAAACACACAAAATGG | 7896 |
| hs132673994 | TGTCTCCTCTTTTTAATAGAAGG | 7897 |
| hs132672360 | TGTTTCTCCTGGATAAATATAGG | 7898 |
| hs132674249 | AGGAATGTCAGTAAATTTAATGG | 7899 |
| hs132675049 | GCAGCTATATGGAGCACATTTGG | 7900 |
| hs132674643 | CCAGCAGTGCTCATTTCTATAGG | 7901 |
| hs132675218 | TCATCTGTATCATACCTATTTGG | 7902 |
| hs132673365 | TCTCAGTAGAACATTAAAAATGG | 7903 |
| hs132673749 | GAACATGACTAAAATCTTAAAGG | 7904 |
| hs132673958 | TTTAGCTTAGTTCTTCTGTTTGG | 7905 |
| hs132673957 | AACAGAAGAACTAAGCTAAATGG | 7906 |
| hs132673080 | ACTTTGTGTTCAAGGTAAAATGG | 7907 |
| hs132673890 | GGAGCAAAACGACCACAAATAGG | 7908 |
| hs132672985 | AAGCTGAAGTGCTACATTAATGG | 7909 |
| hs132673629 | AGATTTGCTGAAGGCTTTTAGGG | 7910 |
| hs132675376 | CCACATCATAATCAGAAAATAGG | 7911 |
| hs132673606 | TTTCTAGATAACTATTAAAATGG | 7912 |
| hs132673872 | CCATCCACTTGATAAAATAATGG | 7913 |
| hs132674201 | AATAGTCTGACACTCTAAATTGG | 7914 |
| hs132675460 | TCATTCATATACTCTATAAAAGG | 7915 |
| hs132673108 | CCTTTTGGTCAGGTGCAATTAGG | 7916 |
| hs132673976 | AATAAATCTGCATTGAAAAATGG | 7917 |
| hs132673307 | TCTGATGTATTTTATCCTATTGG | 7918 |
| hs132675466 | ATAGGGCATACACATTTTAAAGG | 7919 |
| hs132673244 | CACATCGCTTTATTGTAAAATGG | 7920 |
| hs132672052 | AATTAAACAAATACACAAAATGG | 7921 |
| hs132672356 | TGAGAAGCATTGTTACTAAATGG | 7922 |
| hs132674362 | TAGCGCCCTCAAATACATTTAGG | 7923 |
| hs132673171 | TTAAGGGATTAAGGAGAAAATGG | 7924 |
| hs132673800 | TTACACTCTCTGAATGCTTTTGG | 7925 |
| hs132672494 | TGGCTTCCACCATTGTAATTAGG | 7926 |
| hs132672338 | CATTGATGTGATATTCTAAAAGG | 7927 |
| hs132673819 | CCAGTTATCCAGAAGATTAAAGG | 7928 |
| hs132674543 | GCATGAAAGCTGATGTTTTATGG | 7929 |
| hs132674097 | ATGCCATCTTATTTGTTAATAGG | 7930 |
| hs132675458 | ATAGAGTATATGAATGAAATTGG | 7931 |
| hs132672577 | TGGAAGCTATACCAAGAAAATGG | 7932 |
| hs132674113 | CTTGACATCTTTGTATTAATAGG | 7933 |
| hs132672713 | TTATTGCTCTTTAAAATATATGG | 7934 |
| hs132672438 | TCATGTGACATAAATTTAATTGG | 7935 |
| hs132675118 | TGTGAGATATGCCCTTTTATAGG | 7936 |
| hs132674167 | TGCGGAGTAGTTTTGTTTAATGG | 7937 |
| hs132674547 | ATAAAATGAATGAAGCTAAATGG | 7938 |
| hs132675279 | GTAGAAAGCCATTTCCATTTAGG | 7939 |
| hs132674912 | GCAAAGATATTATCATTTAATGG | 7940 |
| hs132674977 | TTTTCTTTGCATTAACTAAAAGG | 7941 |
| hs132672823 | AGGAAATCCTTTCAAAAAATTGG | 7942 |
| hs132673598 | TGACAAACCATGAAATAATTTGG | 7943 |
| hs132672134 | TTATATTCAAAGTATTTTAATGG | 7944 |
| hs132672138 | TTAAAATGTGTTCCTCTAATTGG | 7945 |
| hs132672490 | GTATCTTTGTAATTTGAAAATGG | 7946 |
| hs132675085 | CCAATGAAAGACATTTTTATTGG | 7947 |
| hs132671958 | AAAACCATAAGTAACCCGAAAGG | 7948 |
| hs132671949 | ATCACGGTCAGTCTAGCACAGGG | 7949 |
| hs132671973 | GAGCATGCTATTTAGCCATGAGG | 7950 |
| hs132671959 | GAAAGGACTACTTTCCAACATGG | 7951 |
| hs132671995 | GTTTGTGCCTGAGAAAGCATAGG | 7952 |
| hs132671948 | ATCTGCAGAGAAGGGTTTGGGGG | 7953 |
| hs132671985 | AATTCATCAGGTCCATACCCTGG | 7954 |
| hs132671944 | TGACCGTGATCTGCAGAGAAGGG | 7955 |
| hs132671968 | GAAGGCACTTAATATCATGTTGG | 7956 |
| hs132671977 | ACTCCAACATTTATATGGGATGG | 7957 |
| hs132672008 | TTTCATTGTGTGTTACACAATGG | 7958 |
| hs132671989 | GAAATTTTTAGTCCTTTCCAGGG | 7959 |
| hs132671950 | GATCACGGTCAGTCTAGCACAGG | 7960 |
| hs132671979 | TGGGATGGTACTCTTCAATCCGG | 7961 |
| hs132671980 | GTACCATCCCATATAAATGTTGG | 7962 |
| hs132671955 | GGGGGAGTGGATAGAGAGGAGGG | 7963 |
| hs132671999 | TTCTTTCTCTCAACAGTGGGTGG | 7964 |
| hs132671993 | TGAATGTGTGGAGAGCTCTTAGG | 7965 |
| hs132672002 | GTGGGGTTCTTCCATGACATTGG | 7966 |
| hs132672004 | ATAATTGTATAGAATTTAGTGGG | 7967 |
| hs132672011 | ATTCATATGTACCATATCATAGG | 7968 |
| hs132671982 | TTATGGATGCCTGTTTTTGCCGG | 7969 |
| hs132671946 | TGATCTGCAGAGAAGGGTTTGGG | 7970 |
| hs132671966 | ATATAAATGATATTCGAAATTGG | 7971 |
| hs132672010 | TGTTCTATATTCCTATGATATGG | 7972 |
| hs132670257 | CAGACACTGATATCACACTGAGG | 7973 |
| hs132671162 | TCTCACAAGTAAGCTCACTGGGG | 7974 |
| hs132670891 | CAAAACCTAAGCAAACCCTGGGG | 7975 |
| hs132671289 | TAATTGGAGTTATTCCACAGGGG | 7976 |
| hs132670076 | GAAGCCAAGTAATAGAGACGGGG | 7977 |
| hs132670047 | GCATCAGTAAGAAAACAAGGAGG | 7978 |
| hs132670717 | ACATATGAGTGTATACACTGAGG | 7979 |
| hs132670065 | GTGTGTAAGAGCAAGCAAGGAGG | 7980 |
| hs132670299 | GTACTGTGCTGTTAGGCACGGGG | 7981 |
| hs132670673 | TGGGCACTATAAATATGACAAGG | 7982 |
| hs132671813 | TATAAGAGGAGAGAGCCATGTGG | 7983 |
| hs132670286 | GTGGCACTAACAAAAACACAAGG | 7984 |
| hs132670256 | TCAGCAACGTAATGAAGACGAGG | 7985 |
| hs132671620 | AAGAAATATTTACAACAACGTGG | 7986 |
| hs132670703 | GCATAAGATGGAAATCCTAGGGG | 7987 |
| hs132670134 | ATGTAGCACATACACACCAGGGG | 7988 |
| hs132670885 | ATGTGTACTTCTAAACAGTGAGG | 7989 |
| hs132671421 | GAGACAGTGAACCAAGCATGGGG | 7990 |
| hs132671501 | TGAGAAATAAGATTAGCCCGTGG | 7991 |
| hs132671040 | AATTTGGAGCCTGATATGAGTGG | 7992 |
| hs132671843 | GAGCGATGCCTTAATGAAGGTGG | 7993 |
| hs132670086 | ATACACTCAAGTGACAGGAGAGG | 7994 |
| hs132671001 | CACATGTTACTGCTCCAATGTGG | 7995 |
| hs132670117 | ACGGGGCCCCCTAACAAACAGGG | 7996 |
| hs132670111 | CATAGGCATACTATTTACGGAGG | 7997 |
| hs132671519 | GTAATGATGCCAATGATGAGAGG | 7998 |
| hs132671541 | ATACATGTGTTAAATCCATGTGG | 7999 |
| hs132671899 | ACATTGCCGAGAATATGACCTGG | 8000 |
| hs132671280 | AATATTGCTAGGTATGCTAGGGG | 8001 |
| hs132670768 | GATTATGGGAAACTGGAAGGAGG | 8002 |
| hs132671810 | ATAGGGGACAGTCACCCACATGG | 8003 |
| hs132671595 | GGTGTTTAAGCAGGTCAGGGCGG | 8004 |
| hs132670784 | GTGCTGCAAATGAATTGAGGAGG | 8005 |
| hs132671078 | AAGTGAATATATGCTATGAAGGG | 8006 |
| hs132670663 | AATTGATGAGATATATACTGAGG | 8007 |
| hs132670833 | GGTTGAAGGACACATTGTGGAGG | 8008 |
| hs132671580 | AGGTGAGCTAAGCATTACACAGG | 8009 |
| hs132670181 | GACTTAAGAAGGTAATCTTGGGG | 8010 |
| hs132671317 | GTGAGTAAAATTGATTGACGTGG | 8011 |
| hs132670023 | CCACTGGCATTATCAAACACAGG | 8012 |
| hs132670929 | ATACATCAGGTTGACCTGGGAGG | 8013 |
| hs132671611 | GATACCAGAGATAGGTCCAGAGG | 8014 |
| hs132670842 | GTAGGGCATATGGTTAGGGGAGG | 8015 |
| hs132671628 | TATCACATAGAGTGTTAGAGGGG | 8016 |
| hs132671357 | ACTCCCGTCTCAAATATAAGGGG | 8017 |
| hs132670211 | GATGATAAAAACATATTTTGGGG | 8018 |
| hs132671854 | AATAAAGTGATAATGAGAGGGGG | 8019 |
| hs132670669 | AACAGAAGTAGGGAAAGAGGGGG | 8020 |
| hs132671313 | ATACTATTTTGTTTACTGGGGGG | 8021 |
| hs132670672 | TCTTAGAGTGTATTGGACGTGGG | 8022 |
| hs132670337 | TTACAAACACCCTAAAAGGGGGG | 8023 |
| hs132670024 | CCTGTGTTTGATAATGCCAGTGG | 8024 |
| hs132671832 | GCACAAATCACAAGAGATGGGGG | 8025 |
| hs132670136 | AATGTAGCACATACACACCAGGG | 8026 |
| hs132671255 | TGACTGTGAGAGAATGAGGGAGG | 8027 |
| hs132670449 | AGGGAGAGAAATCCCTATGGGGG | 8028 |
| hs132671610 | TAGGTCCAGAGGTATTGCTGTGG | 8029 |
| hs132671636 | ACATGAATAGAATAATTCAGAGG | 8030 |
| hs132670977 | ACTTATGCATGGGATTGGGGAGG | 8031 |
| hs132671759 | GCATGAGGGAAGACAGCAGGAGG | 8032 |
| hs132670115 | AAATATTGCAAGCAAGCACGGGG | 8033 |
| hs132671794 | AAGACACAGAATAGTCAGAGAGG | 8034 |
| hs132670869 | ATAGTGGGAAGCTTGATTGGTGG | 8035 |
| hs132670689 | TGTGTGACACGATAAATTGGAGG | 8036 |
| hs132671459 | AATAAGTCATTATTACTCAGTGG | 8037 |
| hs132670278 | GTATCATACGTACACATATGGGG | 8038 |
| hs132671898 | TCTATATGAGGAAGACTCAGTGG | 8039 |
| hs132671746 | ATATATAGCAGTTACTACAGTGG | 8040 |
| hs132671923 | AAACAAACCAGCTCTTCACGAGG | 8041 |
| hs132671092 | CTATGTACTTTGCTTCAGGGAGG | 8042 |
| hs132671037 | GTTATAAGCCACAGGCAGAGAGG | 8043 |
| hs132670418 | GCATTCGGGAAACTTTAAGTCGG | 8044 |
| hs132671546 | GCTGCCAGAATTCTATAGCAGGG | 8045 |
| hs132671080 | GCAATGTTGGATATTCAAAGAGG | 8046 |
| hs132671838 | ATTTGGTAATATTTACACAAAGG | 8047 |
| hs132671853 | CAATAAAGTGATAATGAGAGGGG | 8048 |
| hs132670862 | TCTAGTGCATAAAGAACCTGAGG | 8049 |
| hs132670336 | CTTACAAACACCCTAAAAGGGGG | 8050 |
| hs132670857 | GCTATAATTTATGGTCCCAGAGG | 8051 |
| hs132671328 | CTTACTCTACTCAGTGCCTGAGG | 8052 |
| hs132670175 | TATCAAAGTCAGCATTAAGGGGG | 8053 |
| hs132671822 | AGAGCAGGTACAGCCTGTAGGGG | 8054 |
| hs132670613 | CAATATTTTTGACAGTACAGAGG | 8055 |
| hs132671688 | AGAAAGTAATTGGCAAACAGAGG | 8056 |
| hs132670145 | TTAAGTCCTTCGAGATGACAAGG | 8057 |
| hs132671627 | AACACTCTATGTGATAGTGGAGG | 8058 |
| hs132670126 | AGAACAACAGAAGCTGTTTGGGG | 8059 |
| hs132671398 | GTAACTCTTCTATTACGATGAGG | 8060 |
| hs132670901 | ATATATACTCAGCTACAAGTGGG | 8061 |
| hs132670167 | ATTGCCTTACTGTTAGACAAGGG | 8062 |
| hs132671306 | TCAAAGATCTCTAAAGATGGGGG | 8063 |
| hs132671381 | CTTGAACAGAGCTTTTTACGAGG | 8064 |
| hs132670150 | ACTATGCCTTGTCATCTCGAAGG | 8065 |
| hs132671339 | AGAGACAGCAAACAATGTTGAGG | 8066 |
| hs132671093 | GTACATAGCAGTATCCCACTTGG | 8067 |
| hs132670415 | ATCATGCTGCATAAAACCAGTGG | 8068 |
| hs132670902 | ATACTCAGCTACAAGTGGGTCGG | 8069 |
| hs132670776 | GTATACTTGTGTGCTGTGAGAGG | 8070 |
| hs132671875 | AGTAAACTAGCCAAGGATGGAGG | 8071 |
| hs132670186 | TTGCTCTTACACATTTTTGGGGG | 8072 |
| hs132671693 | GAGGGACAAGTTGTGCCAGGAGG | 8073 |
| hs132671733 | TCAAACGACAGTAAAAGTGAAGG | 8074 |
| hs132671809 | CATGCACGTGTTTTCCATAGGGG | 8075 |
| hs132670629 | GGATATCTATCCTTGCCTTGAGG | 8076 |
| hs132671397 | ATAAAAGAATGAGTAATACAAGG | 8077 |
| hs132671288 | ATAATTGGAGTTATTCCACAGGG | 8078 |
| hs132670760 | AATTTTGATGATACACTCTGTGG | 8079 |
| hs132671722 | GCTCTTAAAAGTAGATGTAGGGG | 8080 |
| hs132671105 | GAGTTTCCAAGATCTGCTGAGGG | 8081 |
| hs132671653 | AATACATCATTTATTCCACTAGG | 8082 |
| hs132671499 | GTAGAAGTAGAAGAATCCACGGG | 8083 |
| hs132670505 | ATCCTGTTGGCTGAAAGCAGTGG | 8084 |
| hs132671129 | TGTATTATCTTACTACAACTAGG | 8085 |
| hs132671081 | ATGTTGGATATTCAAAGAGGAGG | 8086 |
| hs132670755 | GCACATTGTATGTTATACACAGG | 8087 |
| hs132670775 | TATACTTGTGTGCTGTGAGAGGG | 8088 |
| hs132671911 | TAATGGTTATAAAAAACATGAGG | 8089 |
| hs132671650 | CTAAATGCAGACACACCTAGTGG | 8090 |
| hs132670146 | TTCGAGATGACAAGGCATAGTGG | 8091 |
| hs132671537 | TAGTAGATTTGAGACCCACATGG | 8092 |
| hs132670691 | GCATTAAGCAACACCAACAAGGG | 8093 |
| hs132670736 | ATGATAAAATAAGTATCAGGCGG | 8094 |
| hs132671812 | ATAAGAGGAGAGAGCCATGTGGG | 8095 |
| hs132670863 | AGGTTCTTTATGCACTAGAGAGG | 8096 |
| hs132671471 | TCAGTGCTATCTTTACAGCATGG | 8097 |
| hs132670031 | AGCTCTTCTAGATTACTGGAAGG | 8098 |
| hs132670439 | AATTTAGTGATATATGTGAAAGG | 8099 |
| hs132671206 | CTAACATTACTAATTACTGAAGG | 8100 |
| hs132671414 | TACAATACAATCGCAAACTGGGG | 8101 |
| hs132670072 | AGAGGCTGGAGCGCTCTGCGTGG | 8102 |
| hs132671354 | TATATTTGAGACGGGAGTGGGGG | 8103 |
| hs132671212 | TTACGGACAATGATTAACACTGG | 8104 |
| hs132670623 | AGATTGATGATAATCTTGAGTGG | 8105 |
| hs132671540 | TACATGTGTTAAATCCATGTGGG | 8106 |
| hs132670106 | GCTCAAGAGGGACTTTGCTGAGG | 8107 |
| hs132671028 | GGTTTAAAGAGGCCAGTAGGTGG | 8108 |
| hs132671749 | TACCAGATATGCTATGCTAGAGG | 8109 |
| hs132670923 | GGAAAATGAGTAATTAGCAGAGG | 8110 |
| hs132671773 | CATCACTCGTTCTCCCACACAGG | 8111 |
| hs132671893 | GCCTTATAAATGTTACAGCGTGG | 8112 |
| hs132671110 | TGAGTTCGAAAAGTTATGGAGGG | 8113 |
| hs132671175 | AATCTACCAGATTTTCAACATGG | 8114 |
| hs132671536 | TTACATTGACATGCTTAAGGGGG | 8115 |
| hs132670944 | AGTATGTAACTAACTCAAGTGGG | 8116 |
| hs132671660 | GTTATAGTCCAAAAACCTAGAGG | 8117 |
| hs132671242 | AAGCAGATGATGATGCCCTATGG | 8118 |
| hs132670028 | TGAAAATTGATGTATGCATGAGG | 8119 |
| hs132670909 | AGAACATTATATAAGTACCTAGG | 8120 |
| hs132670804 | GTGATAGAATCGCATTGCAGGGG | 8121 |
| hs132670470 | TGAGAGGTGATGTAGCATCTCGG | 8122 |
| hs132670022 | AAAATGCTAGGACTGTCCACTGG | 8123 |
| hs132670990 | CTTAACATGTTCCAACCAGAAGG | 8124 |
| hs132670297 | CCCGCCTTCCTACAATGCAAGGG | 8125 |
| hs132671871 | AGAGTCGATGGAGAGTCAAGAGG | 8126 |
| hs132670534 | CTTTTTTGTAATAACTCAAGGGG | 8127 |
| hs132671902 | GAAAAAGTCACACTGAAACCAGG | 8128 |
| hs132670716 | CATATGAGTGTATACACTGAGGG | 8129 |
| hs132670258 | GAATACCAGAAAGACAGCAATGG | 8130 |
| hs132670374 | ATGGACTACGGTCATTCTAGAGG | 8131 |
| hs132671456 | ATATTGCCTCAATATTTCAAGGG | 8132 |
| hs132671476 | ACTTGAATAGTGATCACCATAGG | 8133 |
| hs132670791 | ATCTGTAGGGAACTAGAGGTGGG | 8134 |
| hs132670413 | ATTGTTATATACAGCAATGGAGG | 8135 |
| hs132670628 | GCTGATGGCATCGAATGCATTGG | 8136 |
| hs132671218 | TATATATGTACTTAGAACAAAGG | 8137 |
| hs132670332 | GTAAGAACAAAGAAATCACTAGG | 8138 |
| hs132671299 | CGAAAGTTCTTAAAGTCTAGGGG | 8139 |
| hs132671566 | GCACATAAGTGTCACAGAGTAGG | 8140 |
| hs132671086 | ACAATGCTTTCCAAATCACCAGG | 8141 |
| hs132670729 | TGAATAATGCAGAGATGCGAAGG | 8142 |
| hs132670785 | ACAGTGCTGCAAATGAATTGAGG | 8143 |
| hs132671386 | GCAGTTTATGCATCCTACAATGG | 8144 |
| hs132670676 | AGACAAAGCAAGGTTTTGTGAGG | 8145 |
| hs132670808 | ATACAGTTAGCTTATTCTGGAGG | 8146 |
| hs132670095 | TAAACTACTAAGTTTGCAGGAGG | 8147 |
| hs132671524 | ATGCAGATGATCTAGAAATGAGG | 8148 |
| hs132671690 | ACAATATGTGGACAGTCTGAGGG | 8149 |
| hs132671243 | TGTTAGCAAGAAACACCATAGGG | 8150 |
| hs132670397 | CCAATGAGAATCAATTGGAGGGG | 8151 |
| hs132670504 | GACTTTTATTACTTTCCTTGAGG | 8152 |
| hs132670474 | TTCCTAAGAGAATTAGACCAAGG | 8153 |
| hs132671355 | ATATAAGGGGCGTAGCCTGAGGG | 8154 |
| hs132671076 | AGTCTTTATGATCCATCCAGTGG | 8155 |
| hs132671157 | ATTATCTAAGATCAACACTCAGG | 8156 |
| hs132671256 | CTCTGACTGTGAGAGAATGAGGG | 8157 |
| hs132670545 | AATATGCAGCAAATATTACTGGG | 8158 |
| hs132670335 | TCTTACAAACACCCTAAAAGGGG | 8159 |
| hs132670988 | TTAACACAGTATATTAACTGTGG | 8160 |
| hs132671686 | GTAATTGGCAAACAGAGGATGGG | 8161 |
| hs132671049 | GCTGCTTCTTCATGATAGGAGGG | 8162 |
| hs132670321 | CCTTTCTCATAAAATCAAGAAGG | 8163 |
| hs132670074 | CTGAAGCCAAGTAATAGAGACGG | 8164 |
| hs132670236 | AATTAAGCCTCTGAAGTCTGTGG | 8165 |
| hs132671588 | TACTGCATCTAGCCTACATGTGG | 8166 |
| hs132671022 | AAAAACAGTTGATTAAACAATGG | 8167 |
| hs132671663 | CTAACTCAGAGCTATTAGGAGGG | 8168 |
| hs132670401 | GCTTGGACATGCAATAGAGTGGG | 8169 |
| hs132671103 | TTACATTTAGCTGTAAACAAGGG | 8170 |
| hs132671866 | AGTCTTGGTGATTTCAACGGAGG | 8171 |
| hs132670950 | TTTATCATATAAAGACCTTGAGG | 8172 |
| hs132671377 | AGAATGAAAATACTTTAGGTGGG | 8173 |
| hs132671666 | TTACTATTTTAAATACATCAGGG | 8174 |
| hs132670627 | CATTCGATGCCATCAGCACAGGG | 8175 |
| hs132671797 | TGAGAAAAATGTTTCATCAGTGG | 8176 |
| hs132670523 | AACGCTAATAAATAAGAGAGAGG | 8177 |
| hs132670434 | GCAACTGGTGTATGTTAAGGAGG | 8178 |
| hs132670854 | TTGGCTTAGCAGGTAGCAGAAGG | 8179 |
| hs132671089 | GGTTTTTGTGCAAGCCAAGTGGG | 8180 |
| hs132670240 | GCGATGTCTTCAAGGAACATAGG | 8181 |
| hs132671756 | GTTAAAAACGTATGGCATGAGGG | 8182 |
| hs132671550 | CAGGGATTAAAATTAGGCATGGG | 8183 |
| hs132671886 | GCCAATTCGAAACGTGCTAGAGG | 8184 |
| hs132670178 | AACACTTCTTATATATGCAGAGG | 8185 |
| hs132671472 | GTAAAGATAGCACTGATACATGG | 8186 |
| hs132671061 | TCTGAAGTTGACATATTTTGGGG | 8187 |
| hs132670557 | ATGGTAGCTGATTCCACTAGAGG | 8188 |
| hs132670889 | CATATAAAGTACATTCCCCAGGG | 8189 |
| hs132670375 | ATTACAAACTCTATGGACTACGG | 8190 |
| hs132670155 | CGAACTTCATCATATCAAAGAGG | 8191 |
| hs132670725 | GAGAAATCATGTTTTTGACAGGG | 8192 |
| hs132670622 | ACTGAAAACCAAATTTGCAAAGG | 8193 |
| hs132671873 | CTTTGCAAGTAAACTAGCCAAGG | 8194 |
| hs132671859 | TACACCAAGCAAAGATATAGGGG | 8195 |
| hs132671075 | CTTAGTCATAGTCCACTGGATGG | 8196 |
| hs132670822 | ATTCAGCTTCATAAGTCCCAGGG | 8197 |
| hs132670500 | ACTTTCCTTGAGGCAGAGGGTGG | 8198 |
| hs132670530 | TCACTATACTTCAGTTCTGGTGG | 8199 |
| hs132671776 | TTAATTTTGTCCATATAACAAGG | 8200 |
| hs132670169 | AGGGGTTAGAACAACTTCCATGG | 8201 |
| hs132670668 | AAACAGAAGTAGGGAAAGAGGGG | 8202 |
| hs132670166 | GCAATTTCTGAAGGTCTTAGGGG | 8203 |
| hs132670104 | TCCTGAGAACATGCTCAAGAGGG | 8204 |
| hs132670867 | CATAATAGTGTCATATGTGAAGG | 8205 |
| hs132671799 | TTCTCAGCCTCTACACTCAGCGG | 8206 |
| hs132670293 | ACCCTTGCATTGTAGGAAGGCGG | 8207 |
| hs132670794 | GATCGATCTATCTATCTGTAGGG | 8208 |
| hs132671729 | CTGAATAGTTGATGGCCACAGGG | 8209 |
| hs132670675 | CAGCAAGCAAAGACAAAGCAAGG | 8210 |
| hs132671158 | TCTAAGATCAACACTCAGGAAGG | 8211 |
| hs132670877 | TTAAAATTCCAAATAGAACAAGG | 8212 |
| hs132670174 | CTATCAAAGTCAGCATTAAGGGG | 8213 |
| hs132671819 | GTAGAGCAGGTACAGCCTGTAGG | 8214 |
| hs132671104 | AGAGTTTCCAAGATCTGCTGAGG | 8215 |
| hs132670402 | AGCTTGGACATGCAATAGAGTGG | 8216 |
| hs132670357 | TCATGAATCTCTGTCAGCCAAGG | 8217 |
| hs132671270 | TTATTGCCAAAGTTGTAAGGAGG | 8218 |
| hs132670533 | ACTTTTTTGTAATAACTCAAGGG | 8219 |
| hs132671788 | ATTGAATACAGATTGAAGCCAGG | 8220 |
| hs132670070 | GAGAAAGGCTTCCTCTGGAGAGG | 8221 |
| hs132670759 | TTGATGATACACTCTGTGGTAGG | 8222 |
| hs132670812 | TTGACACTTTCAACTCAGCATGG | 8223 |
| hs132670538 | TTGGTCACAGAAATTTCTAAGGG | 8224 |
| hs132670273 | CTTCTTAACTGGAGCATCGGAGG | 8225 |
| hs132671725 | ACATGATTAGTTATCAGGATGGG | 8226 |
| hs132671422 | AGAGACAGTGAACCAAGCATGGG | 8227 |
| hs132671820 | ACTTCAAAGGAACTCAAGACAGG | 8228 |
| hs132671629 | CTATCACATAGAGTGTTAGAGGG | 8229 |
| hs132670665 | ACTTCAACAAAACAGAAGTAGGG | 8230 |
| hs132670326 | AATTCTCCTAAATCAATGGAAGG | 8231 |
| hs132670396 | CAATGAGAATCAATTGGAGGGGG | 8232 |
| hs132670931 | TTTGTAAAGGACAAGAATAGGGG | 8233 |
| hs132670956 | AGATGACTTTGATTGATAGAGGG | 8234 |
| hs132670501 | ATAAAAGTCCTCTATCCTGTTGG | 8235 |
| hs132671036 | TTATAAGCCACAGGCAGAGAGGG | 8236 |
| hs132670059 | ATTTAGCCTTTATTTCCAGAAGG | 8237 |
| hs132671522 | GGATTTGCTACACATAAAAGAGG | 8238 |
| hs132671874 | GCAAGTAAACTAGCCAAGGATGG | 8239 |
| hs132670027 | ATACATCAATTTTCAAATAGCGG | 8240 |
| hs132670897 | ATATAGATGACAGTTTATGTTGG | 8241 |
| hs132670120 | ACAAAACCCTGTTTGTTAGGGGG | 8242 |
| hs132670742 | AGTTTTATCAGAGAGCAAGATGG | 8243 |
| hs132671867 | GTCTTGGTGATTTCAACGGAGGG | 8244 |
| hs132671035 | TATAACGTGAACGCCTTGAGAGG | 8245 |
| hs132670602 | CAACAAGAAAGTTAAGGCAAAGG | 8246 |
| hs132670292 | CTAACCCTTGCATTGTAGGAAGG | 8247 |
| hs132670234 | ACGTTATCCACAGACTTCAGAGG | 8248 |
| hs132671348 | GTTTTGAACAGAAAACCCTCAGG | 8249 |
| hs132670765 | GTTCAGTGATGAAAGTAAGGAGG | 8250 |
| hs132671458 | TATTTGTTTAAAAATTAAGACGG | 8251 |
| hs132670805 | AGTGATAGAATCGCATTGCAGGG | 8252 |
| hs132671072 | TGCTAAGTAAAAAGTTAGAAGGG | 8253 |
| hs132671705 | TCCAGCAATCCTGTGTTGGGAGG | 8254 |
| hs132670879 | AATTACATCTTCTCAACCAGTGG | 8255 |
| hs132671265 | GTCAAATTTTCTCTTCACAAAGG | 8256 |
| hs132670829 | TTATTCAGTAGGCAAGAGAAGGG | 8257 |
| hs132670404 | GGGCAGTGAGATTTCAGAGATGG | 8258 |
| hs132671166 | TTATGCTCTTATGTATAAGAGGG | 8259 |
| hs132670746 | GGAGGAAAATACAGAGACTACGG | 8260 |
| hs132671029 | CTTGGTTTAAAGAGGCCAGTAGG | 8261 |
| hs132671716 | TTTCCTTAGAATCTTTTACTCGG | 8262 |
| hs132671740 | TTTAATAAGAACAATTTCAGTGG | 8263 |
| hs132670446 | ATAAGGGAGAGAAATCCCTATGG | 8264 |
| hs132671149 | CAGGCAACCAGAAAATAAGAAGG | 8265 |
| hs132671889 | GCGTGGGAATGAAATGTGCTAGG | 8266 |
| hs132671189 | AGAATTTCATTGCCATTTTGGGG | 8267 |
| hs132671039 | GCGTTCACGTTATAAGCCACAGG | 8268 |
| hs132671594 | GCCCTGACCTGCTTAAACACCGG | 8269 |
| hs132671374 | ATAACCCTATGCTTAAGAGATGG | 8270 |
| hs132671879 | GTACACATTTTTTAGCCGATAGG | 8271 |
| hs132670239 | ACATCGCTCATTAAAAAAGGTGG | 8272 |
| hs132670958 | ATCAAAGTCATCTTTAGCATAGG | 8273 |
| hs132671892 | CCTTATAAATGTTACAGCGTGGG | 8274 |
| hs132671861 | TGCTCCTTAATCTTAGGAGGTGG | 8275 |
| hs132671464 | TAGTTATGAGGATGAGACTAGGG | 8276 |
| hs132670756 | AATGTGCTATATAAAATATGTGG | 8277 |
| hs132671340 | GTGGAAGGAGAGTTGATGGGGGG | 8278 |
| hs132670295 | CCTTGCATTGTAGGAAGGCGGGG | 8279 |
| hs132671147 | TAAGAAGGTAAAATACAGGCAGG | 8280 |
| hs132671679 | GATACTTTTAATCTAAGGAGTGG | 8281 |
| hs132670719 | CCACAAAGTTGATAGGCATAGGG | 8282 |
| hs132671498 | AGTAGAAGTAGAAGAATCCACGG | 8283 |
| hs132670747 | GGCATTGAAGAGAATTAAGTAGG | 8284 |
| hs132670030 | AATACAATATTAATTTGGTGTGG | 8285 |
| hs132670696 | CCATTCTTCTAGTTTTATGGAGG | 8286 |
| hs132671845 | CCTTCATTAAGGCATCGCTCAGG | 8287 |
| hs132670618 | GCAGGTGATATCGCTAAAGATGG | 8288 |
| hs132671842 | CCTGAGCGATGCCTTAATGAAGG | 8289 |
| hs132670778 | GGAGGATGCAGGTAGGGGAAGGG | 8290 |
| hs132670678 | GAGCTCATTGATGTCATCCAAGG | 8291 |
| hs132670708 | TGGTGAGAACTTTTAAGCGAAGG | 8292 |
| hs132670355 | CTCTGTCAGCCAAGGAATGTCGG | 8293 |
| hs132670651 | ACCAGTATCCTGATATATGTTGG | 8294 |
| hs132670409 | ACAGCAATGGAGGTAGGGCAGGG | 8295 |
| hs132671511 | CTTCATTATAACATGTCCCAAGG | 8296 |
| hs132670039 | TTCCTTTAATGCCATTGCAGAGG | 8297 |
| hs132670908 | CCAAATTTCTGAAAGATCCAAGG | 8298 |
| hs132670981 | AGCAATCAGGACTTATGCATGGG | 8299 |
| hs132670948 | TATCATATAAAGACCTTGAGGGG | 8300 |
| hs132670797 | AAGTACAATCAAATTTGAAGAGG | 8301 |
| hs132671248 | TTAAAATATACTAAGCATGTAGG | 8302 |
| hs132670077 | CTATTACTTGGCTTCAGATGGGG | 8303 |
| hs132670792 | TATCTGTAGGGAACTAGAGGTGG | 8304 |
| hs132670619 | GTAATGCATTACTCAATGGCAGG | 8305 |
| hs132671269 | TATTGCCAAAGTTGTAAGGAGGG | 8306 |
| hs132671137 | ATTACCATCTCTTTCAGGAGTGG | 8307 |
| hs132670036 | ATATTTACATACCTCTGCAATGG | 8308 |
| hs132670050 | GTAAGAAAACAAGGAGGTAGGGG | 8309 |
| hs132670177 | GCAGCCTGAGAGATGTGCGCAGG | 8310 |
| hs132671589 | CAGTATGCCTTCTTTAGGAAGGG | 8311 |
| hs132671349 | TACGCCCCTTATATTTGAGACGG | 8312 |
| hs132671273 | TAGTGTGTTAGGTTTATCAGTGG | 8313 |
| hs132670158 | AAGAGGGTAAGTGCACTGAAAGG | 8314 |
| hs132670447 | TAAGGGAGAGAAATCCCTATGGG | 8315 |
| hs132671750 | ATATCTGGTAGCAGAATTGGAGG | 8316 |
| hs132670265 | CAGAAGGCGCAGAGTTCGAATGG | 8317 |
| hs132671450 | TTACCATGATAACCCAATTGTGG | 8318 |
| hs132671205 | CATTACTAATTACTGAAGGATGG | 8319 |
| hs132671058 | CTGAGAAAATTTGCAGCACAGGG | 8320 |
| hs132671282 | TATAACACTGGACTAAGCATTGG | 8321 |
| hs132670398 | CCCAATGAGAATCAATTGGAGGG | 8322 |
| hs132671811 | ATGTGGGTGACTGTCCCCTATGG | 8323 |
| hs132671870 | AATTGAGTCTAGAGAGTCGATGG | 8324 |
| hs132671882 | AATGATAGACCTGATCCTATCGG | 8325 |
| hs132671115 | AGAGTTCAAGGTTTGCTCAAGGG | 8326 |
| hs132671350 | ACGCCCCTTATATTTGAGACGGG | 8327 |
| hs132670677 | AAGCAAGGTTTTGTGAGGCAAGG | 8328 |
| hs132671910 | TATGTAACTTCGAAAAGTGTAGG | 8329 |
| hs132670793 | ATCTATCTGTAGGGAACTAGAGG | 8330 |
| hs132671858 | GTATGCTCCTTAATCTTAGGAGG | 8331 |
| hs132671549 | GCAGGGATTAAAATTAGGCATGG | 8332 |
| hs132671848 | TGTGCTACCAGAATAGGCATTGG | 8333 |
| hs132671607 | GAGTCAATTTTTGCGAACCAAGG | 8334 |
| hs132670193 | TGATTTATTGATATTTACACAGG | 8335 |
| hs132671703 | CAGCAATCCTGTGTTGGGAGGGG | 8336 |
| hs132670767 | GGAAAGTTTAATCAATTCTGAGG | 8337 |
| hs132670274 | TGTCTTCTTAACTGGAGCATCGG | 8338 |
| hs132671112 | CTTTTCGAACTCATACAGACCGG | 8339 |
| hs132671692 | TCTGAGGGACAAGTTGTGCCAGG | 8340 |
| hs132671088 | CGGTTTTTGTGCAAGCCAAGTGG | 8341 |
| hs132670244 | GGAGAAAGGTAAAAAAATCAGGG | 8342 |
| hs132671457 | CAGTGGCCCTTGAAATATTGAGG | 8343 |
| hs132670455 | CTACCCATTAGTTCCCCCATAGG | 8344 |
| hs132670288 | TCAGTATATGAGATTGCTGTAGG | 8345 |
| hs132671387 | GATGAATGGGATCCAAGAATCGG | 8346 |
| hs132670443 | ATGTGGTATATCCTTCATAAGGG | 8347 |
| hs132671865 | TTAAGTCTTGGTGATTTCAACGG | 8348 |
| hs132671225 | TGAAATTAATGTGTGTTCAGTGG | 8349 |
| hs132671085 | GCCATCAGAGAGTAGAAGAAAGG | 8350 |
| hs132671171 | TCCCAGAACAGGTAGTGAAAGGG | 8351 |
| hs132670982 | AAGCAATCAGGACTTATGCATGG | 8352 |
| hs132671375 | AAATGGTATTACTAAAGCCATGG | 8353 |
| hs132671547 | GCACATATCCTTGCATATTGTGG | 8354 |
| hs132671582 | ATAACTGAGTCACACAACTAAGG | 8355 |
| hs132670873 | TTGTACACTTACAATATGGCAGG | 8356 |
| hs132670984 | TAAATAAATCCTTTTTGCCAGGG | 8357 |
| hs132670871 | GCATGTGGCAGTTACTTGAATGG | 8358 |
| hs132671905 | ATTTTTCTGGATCTACGTAAAGG | 8359 |
| hs132670584 | TTTTACAAATTTATTAACACGGG | 8360 |
| hs132671834 | CAGCACAAATCACAAGAGATGGG | 8361 |
| hs132670850 | AAGTGAAGTAATATTTCTATGGG | 8362 |
| hs132670646 | GTAACTTAAGTAAATGTAGGAGG | 8363 |
| hs132671428 | ATTCTAATCGATGGGTCTTGTGG | 8364 |
| hs132671096 | AAACTCTTAGATCATATGCCTGG | 8365 |
| hs132671728 | CTGTGGCCATCAACTATTCAGGG | 8366 |
| hs132671430 | AATCCATAATTCTAATCGATGGG | 8367 |
| hs132670641 | TCTTTGTCTAGCCTATCTACTGG | 8368 |
| hs132670892 | TCAAAACCTAAGCAAACCCTGGG | 8369 |
| hs132671798 | TCTCAGCCTCTACACTCAGCGGG | 8370 |
| hs132670394 | GAGGGGGAGAGATTGGAGAGAGG | 8371 |
| hs132671621 | TGAATGCCTGTTGTGTAAGAAGG | 8372 |
| hs132670229 | ATGAGAGGCATGACAGTGAATGG | 8373 |
| hs132670431 | AACTAGGTAAATTTTCTCCAAGG | 8374 |
| hs132670611 | ATATTGACTGAATTGACTGTAGG | 8375 |
| hs132671763 | ACAAAAGCACTCTGAGCTAAGGG | 8376 |
| hs132671586 | TAAGAGGCCCTTCCTAAAGAAGG | 8377 |
| hs132670301 | GTGTACTGTGCTGTTAGGCACGG | 8378 |
| hs132670511 | ATAGGAGTAATCATGCTTCAGGG | 8379 |
| hs132671739 | TGAAATTGTTCTTATTAAAGGGG | 8380 |
| hs132671823 | CTGTACCTGCTCTACTTCAAAGG | 8381 |
| hs132670282 | TGTTGTGTAGATGGGAGTGATGG | 8382 |
| hs132670502 | ATTACTTTCCTTGAGGCAGAGGG | 8383 |
| hs132670904 | TATATGTATAATTATCTTGGTGG | 8384 |
| hs132671431 | AAATCCATAATTCTAATCGATGG | 8385 |
| hs132671895 | GGTTCCATACAATATTGAAAGGG | 8386 |
| hs132671791 | CTTTAGCTTATGAAACTCATGGG | 8387 |
| hs132670788 | TATGTGATGGTACTTTCCTAAGG | 8388 |
| hs132670707 | TTGTCTATTTGACACTGGGTCGG | 8389 |
| hs132671714 | TTTTTGTTCTGAGGTTGCTGAGG | 8390 |
| hs132671445 | ACTAATGTATCACAATATGCAGG | 8391 |
| hs132670975 | ACAAGATTCCTTCATAAGGATGG | 8392 |
| hs132671488 | TCCTGGCTTCTAGGATCAAGTGG | 8393 |
| hs132671154 | TGAAGCTATCATTAGAGGAAAGG | 8394 |
| hs132671400 | TCTATTACGATGAGGTTGATGGG | 8395 |
| hs132670159 | AGAGGGTAAGTGCACTGAAAGGG | 8396 |
| hs132671662 | TCTAACTCAGAGCTATTAGGAGG | 8397 |
| hs132671517 | TGAGAGGTATGTATAACAATGGG | 8398 |
| hs132670453 | TACCCATTAGTTCCCCCATAGGG | 8399 |
| hs132670881 | TTGAAGTAGAAAATTCTGACAGG | 8400 |
| hs132670937 | ATTTACAGAAACAAACCATAGGG | 8401 |
| hs132670685 | ACTTCTGGTTATTACTGGCCCGG | 8402 |
| hs132670592 | GGGAGTTTATGGTTGTTGTAGGG | 8403 |
| hs132670372 | AGTAAACTGCTTTAGACAAGTGG | 8404 |
| hs132670828 | AGTAGGCAAGAGAAGGGCATTGG | 8405 |
| hs132671808 | GCATGCACGTGTTTTCCATAGGG | 8406 |
| hs132671787 | AACTCAAGTAAGATTGGAAGTGG | 8407 |
| hs132671019 | CCAAGTATCATTTTGAGTTGGGG | 8408 |
| hs132670898 | TAAACTGTCATCTATATTGGTGG | 8409 |
| hs132671044 | ATGTTTGTATGAACAGAGACAGG | 8410 |
| hs132671050 | AGCTGCTTCTTCATGATAGGAGG | 8411 |
| hs132670144 | TCAGAAGAGCACAAGTAAGTTGG | 8412 |
| hs132670694 | TAAGGTCCAATGAAATATGATGG | 8413 |
| hs132670655 | GTACCGAGAATTAAGAATATGGG | 8414 |
| hs132670478 | TCTGTCATGGAAAGATGCCTTGG | 8415 |
| hs132670634 | CCTTATTTCTCCTCAAGGCAAGG | 8416 |
| hs132671697 | GTTGTGCCAGGAGGGGGCAAAGG | 8417 |
| hs132670577 | CAGATAATAACATTGTGCCTAGG | 8418 |
| hs132670276 | GGTATCATACGTACACATATGGG | 8419 |
| hs132670359 | AATAACTCTGTACTGTTTGGTGG | 8420 |
| hs132671715 | GGATATCCTTTTTTGTTCTGAGG | 8421 |
| hs132670092 | AACCTAAAGGCATTTATCAAGGG | 8422 |
| hs132670552 | ATAAGTTGAAATGTACCAATGGG | 8423 |
| hs132671518 | ATGAGAGGTATGTATAACAATGG | 8424 |
| hs132671489 | GCCACTTGATCCTAGAAGCCAGG | 8425 |
| hs132671264 | TTGTGAAGAGAAAATTTGACTGG | 8426 |
| hs132670425 | GAGAACTAACAACAATAAAGAGG | 8427 |
| hs132670506 | GCTTTCAGCCAACAGGATAGAGG | 8428 |
| hs132671236 | TAGTAACTTTGTGTAAAGGTGGG | 8429 |
| hs132670795 | TGATCGATCTATCTATCTGTAGG | 8430 |
| hs132671285 | TATAACTGTAGAAATACAAAAGG | 8431 |
| hs132670296 | AGGGTTAGTGTAATATCTAAAGG | 8432 |
| hs132670740 | TCAGAGAGCAAGATGGCATAGGG | 8433 |
| hs132671202 | ACCATTTTATTTATCTGCACCGG | 8434 |
| hs132670526 | TTAATAGGATGCATAATGTCAGG | 8435 |
| hs132670700 | CTGTTACTGAAAGCATAAGATGG | 8436 |
| hs132671601 | AATTTGAATGAATTATTGGATGG | 8437 |
| hs132670939 | ATTTTCTTCTCACAGATGACTGG | 8438 |
| hs132671642 | AAAAAAGCTTTGAATAGTGTGGG | 8439 |
| hs132670563 | CATATTTTAATTTATGGACGTGG | 8440 |
| hs132670780 | ATTGAGGAGGATGCAGGTAGGGG | 8441 |
| hs132670820 | CATTCAGCTTCATAAGTCCCAGG | 8442 |
| hs132671283 | AAAAGGCTCAACTATAACACTGG | 8443 |
| hs132671130 | ACTACAACTAGGATGCCAAATGG | 8444 |
| hs132671868 | GGTGATTTCAACGGAGGGAATGG | 8445 |
| hs132671384 | TGGTCCATCGACTCGTACTCAGG | 8446 |
| hs132670287 | TGAGATTGCTGTAGGTTTCGTGG | 8447 |
| hs132671916 | ATGAAAGTAGAAACCATTACGGG | 8448 |
| hs132670816 | TTTTTGGCAATCCTTCCTAAAGG | 8449 |
| hs132671801 | TTCTTAATCACAGAGACAAATGG | 8450 |
| hs132670699 | CCTCCATAAAACTAGAAGAATGG | 8451 |
| hs132670259 | AATACCAGAAAGACAGCAATGGG | 8452 |
| hs132670650 | GTATCCTGATATATGTTGGTTGG | 8453 |
| hs132671293 | ACAAAAAACCAGAAAGAGTTGGG | 8454 |
| hs132671734 | GAGGTGTTGCTGATTAGAGTAGG | 8455 |
| hs132671196 | TTTGTGTACACATAGTGTAAAGG | 8456 |
| hs132670204 | AGGCAAATTTCTTTTTTCGTCGG | 8457 |
| hs132671410 | GAGTGAACAGCAATACTTAAGGG | 8458 |
| hs132671164 | ATAATACAAAGCACGTAAAAGGG | 8459 |
| hs132671894 | GGGTTCCATACAATATTGAAAGG | 8460 |
| hs132671592 | TTGTTTGGATCTCCACATGTAGG | 8461 |
| hs132670674 | AGTTGTTGAGAATGATTCCTAGG | 8462 |
| hs132671649 | ATAAATGTAAATTACATGCTAGG | 8463 |
| hs132670151 | TTTTCCAGTTTCTATAAAAGTGG | 8464 |
| hs132671720 | AGGCTCTTAAAAGTAGATGTAGG | 8465 |
| hs132671152 | CTATCATTAGAGGAAAGGTTGGG | 8466 |
| hs132670131 | CCAAAGGTTGAATTTTCTCTGGG | 8467 |
| hs132671074 | TTCACTTAGTCATAGTCCACTGG | 8468 |
| hs132671680 | AGTATCACTAATTTGCTTCGAGG | 8469 |
| hs132671760 | CCTAGAAATACAAATTAAGTTGG | 8470 |
| hs132671370 | TTCGTCCATCTCTTAAGCATAGG | 8471 |
| hs132671006 | ATATAAACTAGAATGATGTTTGG | 8472 |
| hs132671132 | TTAATGCACCATTTTTTCACAGG | 8473 |
| hs132671221 | TTTATATCTTAACTTTGTAGTGG | 8474 |
| hs132670837 | GGAGGAGTAGGAGAGGCGGCTGG | 8475 |
| hs132671051 | AGAAGCTGCTTCTTCATGATAGG | 8476 |
| hs132671743 | GTAACTTTATCGGTCAAAATGGG | 8477 |
| hs132671083 | TCTCTGATGGCACAGTAGCCTGG | 8478 |
| hs132670786 | TTTAAATATCTGTTATGTGATGG | 8479 |
| hs132670670 | TATATGATCTTAGAGTGTATTGG | 8480 |
| hs132671914 | TCTAAGGTTCTCTTTGACAAAGG | 8481 |
| hs132670800 | CTGCAATGCGATTCTATCACTGG | 8482 |
| hs132671239 | TATTTTTTAATCACACATGAAGG | 8483 |
| hs132670475 | GCATCTTTCCATGACAGAGCAGG | 8484 |
| hs132670438 | GTGATATATGTGAAAGGTAACGG | 8485 |
| hs132670436 | AACGGGATAAACCAAGCAACTGG | 8486 |
| hs132670208 | TTAAAATGTCTTTTATTCCAAGG | 8487 |
| hs132670078 | TCTATTACTTGGCTTCAGATGGG | 8488 |
| hs132671222 | TATAAAAGAGTTTAATTGCATGG | 8489 |
| hs132670422 | TTATAAAAGTTGAATGGCAAAGG | 8490 |
| hs132670763 | GATGAAAGTAAGGAGGAGATGGG | 8491 |
| hs132670352 | GTAAACAAACCGACATTCCTTGG | 8492 |
| hs132670349 | TGAGGTGTGGGAGGAAAAATGGG | 8493 |
| hs132670660 | TATTACATACTGTCATTCACTGG | 8494 |
| hs132670433 | CTTAACATACACCAGTTGCTTGG | 8495 |
| hs132670452 | GGGATTTCTCTCCCTTATGAAGG | 8496 |
| hs132671569 | GTACAGATCTTCAAATATCCAGG | 8497 |
| hs132671302 | ACAGAAGGAATCTTAATGTCAGG | 8498 |
| hs132671383 | ACGAGTCGATGGACCATTGTAGG | 8499 |
| hs132671389 | CTTATTACTAGCGTGATGAATGG | 8500 |
| hs132671477 | TTAAAACAGGTTTTCAAAGAGGG | 8501 |
| hs132670219 | AAGATGCCAGTATCATCAACTGG | 8502 |
| hs132671675 | GTAATTTGTTCAGTCACATCAGG | 8503 |
| hs132670581 | ATGCATGTACTTTCAGGCCTAGG | 8504 |
| hs132670600 | GTACCTCAACAAGAAAGTTAAGG | 8505 |
| hs132671131 | CTACAACTAGGATGCCAAATGGG | 8506 |
| hs132671201 | GCACATTTAACAAACTCTTAGGG | 8507 |
| hs132670848 | TGACTGTTCGTTTCGTGTGTAGG | 8508 |
| hs132670421 | TATAAAAGTTGAATGGCAAAGGG | 8509 |
| hs132671173 | AGTTGAGCCATTCCCAGAACAGG | 8510 |
| hs132670300 | TGTACTGTGCTGTTAGGCACGGG | 8511 |
| hs132671371 | TCGTCCATCTCTTAAGCATAGGG | 8512 |
| hs132670865 | TCTAATTTGTGTTAATTACTGGG | 8513 |
| hs132670940 | GGATTGTTATTTAAATTTGGTGG | 8514 |
| hs132670890 | ACATTCCCCAGGGTTTGCTTAGG | 8515 |
| hs132671677 | AGTCACATCAGGATGTATAAAGG | 8516 |
| hs132671754 | TTAAATGAGTTAAAAACGTATGG | 8517 |
| hs132671099 | GGTCTGAGTTTCTCTCTGCTTGG | 8518 |
| hs132671512 | TTAGGTGTCTCTGGGAACTCTGG | 8519 |
| hs132670661 | GAGTTCTTATGGCAGCAGTCAGG | 8520 |
| hs132671052 | TCAAAGATATACTCAAAGATTGG | 8521 |
| hs132671318 | GTCTCAGTATACTGTGAAAAGGG | 8522 |
| hs132671238 | TTTATAGTAACTTTGTGTAAAGG | 8523 |
| hs132670067 | GGAGGTCGATTAGAAAAATCTGG | 8524 |
| hs132670253 | TCAATGGTTTGGAAGCTACTAGG | 8525 |
| hs132671696 | GGACAAGTTGTGCCAGGAGGGGG | 8526 |
| hs132670105 | TTCTCAGGATTTTTGAAGCCCGG | 8527 |
| hs132670591 | TTATGGTTGTTGTAGGGCAGTGG | 8528 |
| hs132670403 | GATTTCAGAGATGGCTAGCTTGG | 8529 |
| hs132671641 | TGATAATTCTAAATAAGAATAGG | 8530 |
| hs132670248 | GTATGATACGACTGTTAACCTGG | 8531 |
| hs132671795 | ATTCTGTGTCTTTCATTACCTGG | 8532 |
| hs132670141 | GTTGGTGCTGGCAGCTTAGTGGG | 8533 |
| hs132670947 | GAGAAACAATAAACCAATGATGG | 8534 |
| hs132671435 | GTTAACTTGCTAAAATTCATGGG | 8535 |
| hs132670959 | TCAGTTGGATTTCTTTCAAAGGG | 8536 |
| hs132671240 | ATTTTTAGAATAATGATATGAGG | 8537 |
| hs132670993 | TTCTCAGTCAAGCAGAACTCTGG | 8538 |
| hs132670451 | ATCCCTATGGGGGAACTAATGGG | 8539 |
| hs132671637 | AATAATTCAGAGGACAAAACAGG | 8540 |
| hs132670632 | CTTGCCTTGAGGAGAAATAAGGG | 8541 |
| hs132670721 | TCTCAGTCCACAAAGTTGATAGG | 8542 |
| hs132671466 | CTATTCGAAGTTATACATATGGG | 8543 |
| hs132670687 | TCAACAATTTATTCATAAAGTGG | 8544 |
| hs132671427 | CGATGGGTCTTGTGGAATAGTGG | 8545 |
| hs132670639 | GTAAGATATCCATAATCAATTGG | 8546 |
| hs132670081 | ACTACCCCGTCTCTATTACTTGG | 8547 |
| hs132670861 | CTTTAAGACACAGTAGAGATAGG | 8548 |
| hs132671167 | GTTATGCTCTTATGTATAAGAGG | 8549 |
| hs132671440 | TATGTTGTCAGAAACATCTAAGG | 8550 |
| hs132671013 | CCCCAACTCAAAATGATACTTGG | 8551 |
| hs132670496 | ATACCATTCTTTGAAATGGCAGG | 8552 |
| hs132670026 | AACAGCAGATACAGACAATAGGG | 8553 |
| hs132671442 | TCTAGAATGACTGGTTTAGATGG | 8554 |
| hs132670101 | TGTACTCCTTGCAGGTCGCCGGG | 8555 |
| hs132671667 | TTTTAAATACATCAGGGCTTAGG | 8556 |
| hs132670749 | AATAATCACTTACTAAAAATGGG | 8557 |
| hs132671612 | TGTCTCTTGATACCAGAGATAGG | 8558 |
| hs132671824 | TGAGCTTTCACTGCCCCTACAGG | 8559 |
| hs132670400 | GTGACCCAATGAGAATCAATTGG | 8560 |
| hs132670272 | CCAGTTAAGAAGACAAAAATGGG | 8561 |
| hs132670210 | GGAAATAAAGATATAAAAAGTGG | 8562 |
| hs132670205 | TATATTTTCCATAGAGACCTTGG | 8563 |
| hs132670945 | CAAGTGGGCTCATCCATCATTGG | 8564 |
| hs132670683 | ATAAACTGAAAGTCAAATGTAGG | 8565 |
| hs132670733 | GCAGGTTATCATATGAATAGTGG | 8566 |
| hs132671271 | GTTTTATTGCCAAAGTTGTAAGG | 8567 |
| hs132671900 | TGTTTAGTACTTTCTATATGAGG | 8568 |
| hs132671214 | TAATAGAGAAAACTGAAGATAGG | 8569 |
| hs132671509 | TTTCAGCCAAAAATTTCCTTGGG | 8570 |
| hs132671470 | ATAATACTAAACACTTACATAGG | 8571 |
| hs132671027 | ACAAAAATGTTGCCACCTACTGG | 8572 |
| hs132670864 | GTGTTACTTGACTTGTCCTCAGG | 8573 |
| hs132670644 | AGAAGAGCTTGAAAATCATATGG | 8574 |
| hs132671918 | ATAAATATGAATTCCCGTAATGG | 8575 |
| hs132671291 | AGTAAAGAACATTTACTGAATGG | 8576 |
| hs132671478 | GTTAAAACAGGTTTTCAAAGAGG | 8577 |
| hs132670773 | AGAAATAATACATAGAATTACGG | 8578 |
| hs132670388 | GGAGTCTTGGATCAAATAACAGG | 8579 |
| hs132671516 | TTATACATACCTCTCATCATTGG | 8580 |
| hs132670762 | ATGATGTTAATATTTCAGATTGG | 8581 |
| hs132670935 | ATTCAGCTTATTAAACCCTATGG | 8582 |
| hs132670042 | TGGAAATATTTCATTACCTAGGG | 8583 |
| hs132670471 | TTTTACCTTATACTGATATGAGG | 8584 |
| hs132670616 | GCTAAAAACAAAGGACTTTGAGG | 8585 |
| hs132671268 | TGACTCCCTCCTTACAACTTTGG | 8586 |
| hs132670571 | GAAACTGTAAATCTGGAATAGGG | 8587 |
| hs132670279 | TCTATAGCCTATTAAACAAAAGG | 8588 |
| hs132670325 | GGCAAATTCTCCTAAATCAATGG | 8589 |
| hs132670423 | GTGATTTTATAAAAGTTGAATGG | 8590 |
| hs132671324 | AATAAGACTGTCTTCACAACTGG | 8591 |
| hs132670809 | CTAACTGTATTTGGAAATCAGGG | 8592 |
| hs132671475 | CCATAGGATTAATAAAACTTAGG | 8593 |
| hs132671495 | TTTGGTACCTCAAATAGTTCAGG | 8594 |
| hs132670608 | TCACACAAAATAACTAAAATGGG | 8595 |
| hs132670706 | CATTTTGTCTATTTGACACTGGG | 8596 |
| hs132670713 | AAATGCTTCAAATTATACTTAGG | 8597 |
| hs132671913 | TCTTTGACAAAGGCTGCTAATGG | 8598 |
| hs132670705 | ACATTTTGTCTATTTGACACTGG | 8599 |
| hs132670724 | TGAGAAATCATGTTTTTGACAGG | 8600 |
| hs132670118 | GTGCTTGCTTGCAATATTTGTGG | 8601 |
| hs132670519 | CATGCTTCCATTTCCTCAGTTGG | 8602 |
| hs132670522 | TTTCTATGTTCACCTCACTTAGG | 8603 |
| hs132670915 | GGTCAATTTTGAAGATAAATGGG | 8604 |
| hs132670726 | ACAGGGCTTGTTTCTCAAGTTGG | 8605 |
| hs132671869 | GTTGAAATCACCAAGACTTAAGG | 8606 |
| hs132670750 | GTATGTTAGTTAAATACTAATGG | 8607 |
| hs132670771 | ATAGAATTACGGCTGATTATGGG | 8608 |
| hs132670507 | TTCCACTGCTTTCAGCCAACAGG | 8609 |
| hs132671493 | ACTTGTTCCTGAACTATTTGAGG | 8610 |
| hs132670122 | TTACAAAACCCTGTTTGTTAGGG | 8611 |
| hs132671814 | AAAATAACGTTCTCTATAAGAGG | 8612 |
| hs132671047 | TTTGTCACAAAAGTGCTTTAGGG | 8613 |
| hs132671432 | GCTCATCTTAGACAGCTATATGG | 8614 |
| hs132670251 | ATTTCCACAAATTCAATCAATGG | 8615 |
| hs132670751 | TATGTTAGTTAAATACTAATGGG | 8616 |
| hs132670657 | CAGCCCATATTCTTAATTCTCGG | 8617 |
| hs132670579 | TATTATCTGAAAAGCTTGATAGG | 8618 |
| hs132671741 | GAGAGAGCATGTAACTTTATCGG | 8619 |
| hs132671860 | ATACACCAAGCAAAGATATAGGG | 8620 |
| hs132671462 | GATTTAGAGTTCTAGTTATGAGG | 8621 |
| hs132670323 | CCTTCTTGATTTTATGAGAAAGG | 8622 |
| hs132670541 | ATATTATAAACATATAAAATTGG | 8623 |
| hs132670247 | AGAATTTCTCTGACTAGAAATGG | 8624 |
| hs132670917 | TTTAAAATAACATTTATATAGGG | 8625 |
| hs132671885 | TCCTCTAGCACGTTTCGAATTGG | 8626 |
| hs132670201 | GTATATGTATATATGTTCTAAGG | 8627 |
| hs132671827 | AGATGATGTTACATGTAGTTAGG | 8628 |
| hs132671482 | GCAATTCTTGCATCATGTATTGG | 8629 |
| hs132671771 | GGAGAACGAGTGATGTAATTTGG | 8630 |
| hs132671358 | CACTCCCGTCTCAAATATAAGGG | 8631 |
| hs132670932 | CTTTGTAAAGGACAAGAATAGGG | 8632 |
| hs132671718 | ATACCGAGTAAAAGATTCTAAGG | 8633 |
| hs132671023 | TTACGAGTACCAACACTATTAGG | 8634 |
| hs132670587 | AGCAATGTTGGCCAACTATACGG | 8635 |
| hs132670874 | TTCATTGTACACTTACAATATGG | 8636 |
| hs132671561 | AATGAAATTTCAAATCCATTTGG | 8637 |
| hs132670311 | TCCATTTTCAAGGTATTGATTGG | 8638 |
| hs132670710 | TTTCTACTCAAGTCAGATAATGG | 8639 |
| hs132670562 | GGCTTTATATTACACTAAAAAGG | 8640 |
| hs132670190 | CTTAAATACGAGCTAGGTTAAGG | 8641 |
| hs132671505 | GAAAGAGGACTACATATCTTAGG | 8642 |
| hs132671534 | CTTTACATTGACATGCTTAAGGG | 8643 |
| hs132670992 | GCAGAACTCTGGACACCTTCTGG | 8644 |
| hs132670485 | AGAATCTCTTTCCTTGAATAGGG | 8645 |
| hs132670477 | TGCCTTGGTCTAATTCTCTTAGG | 8646 |
| hs132670112 | TGAGCAGAACAAACAAATATTGG | 8647 |
| hs132670093 | TGCCCTTGATAAATGCCTTTAGG | 8648 |
| hs132671857 | GGTGTATGCTCCTTAATCTTAGG | 8649 |
| hs132670331 | TTTCTCAGAAATTTATTAGATGG | 8650 |
| hs132671134 | ATAGCAAGCCTGTGAAAAAATGG | 8651 |
| hs132670821 | TGGAGAGGCACTTTGCCTTTAGG | 8652 |
| hs132670758 | CAGAGTGTATCATCAAAATTAGG | 8653 |
| hs132671136 | AGGAGTGGAAGAGAGAGAATTGG | 8654 |
| hs132671644 | ATTTCCTAACCTATTTTGATAGG | 8655 |
| hs132670058 | ATGCTGTTAGAAATTTTAGATGG | 8656 |
| hs132671619 | TTTATAAATATAATGCAAAATGG | 8657 |
| hs132670238 | AAGACATCGCTCATTAAAAAAGG | 8658 |
| hs132671712 | CAGCAACCTCAGAACAAAAAAGG | 8659 |
| hs132670099 | GGGAGGAGTGTACTCCTTGCAGG | 8660 |
| hs132670986 | GTTCTATGTCCCTGGCAAAAAGG | 8661 |
| hs132671046 | GTTCATACAAACATTCGATTTGG | 8662 |
| hs132670636 | TTTTAGTATCTACTCATTAGAGG | 8663 |
| hs132670154 | TCTGCCACTTTTATAGAAACTGG | 8664 |
| hs132671678 | TTAGTGATACTTTTAATCTAAGG | 8665 |
| hs132670680 | TGACTTTCAGTTTATTTAACAGG | 8666 |
| hs132671043 | TTTGGAGTAAAAATACAAATTGG | 8667 |
| hs132671460 | TATGGTGCAATTTTTTTCATTGG | 8668 |
| hs132671448 | TATCATGGTAAAGAGCTTTATGG | 8669 |
| hs132671069 | ATATAATATCTGTTTCAATTTGG | 8670 |
| hs132670686 | AAGTGGCTGCAAATATAATTTGG | 8671 |
| hs132670029 | TTAAAGCTCTTCTAGATTACTGG | 8672 |
| hs132670207 | CTTTTATTCCAAGGTCTCTATGG | 8673 |
| hs132671425 | AGACCCATCGATTAGAATTATGG | 8674 |
| hs132671673 | TTTAGCTCAACTCTATAAATTGG | 8675 |
| hs132671738 | CTGAAATTGTTCTTATTAAAGGG | 8676 |
| hs132670559 | AACTCAGTACATATTCTATTTGG | 8677 |
| hs132670640 | ATAAGTTTACCAATTGATTATGG | 8678 |
| hs132670922 | GCAGAGGTGCAGATTATATTTGG | 8679 |
| hs132670308 | TTTTATATAAAACCTGAAATTGG | 8680 |
| hs132671784 | ACAAAGAGTTGAAAATTAATTGG | 8681 |
| hs132671295 | GTTAAGTGAATGAACAAAATTGG | 8682 |
| hs132670369 | TTAATCAGTCATTTATAAATAGG | 8683 |
| hs132671496 | TTAAAGTCATTTTAACTATTTGG | 8684 |
| hs132671617 | TTACATTTGGTAAAGATATTTGG | 8685 |
| hs132670429 | GTTTGGTTCAATTAGATTTTAGG | 8686 |
| hs132671286 | GCTTCATGATATGGTATAATTGG | 8687 |
| hs132670723 | TTTACTGATGTTTTTCTAAATGG | 8688 |
| hs132669975 | TCATGTACCCCTGACAAAGAAGG | 8689 |
| hs132669980 | GTTAACTTCCTTCTTTGTCAGGG | 8690 |
| hs132670000 | AGGATGTAAGAGAGTAATTGAGG | 8691 |
| hs132669977 | TCTTTGTCAGGGGTACATGATGG | 8692 |
| hs132669986 | CTGGGAATATAAATCTAAATGGG | 8693 |
| hs132669982 | AATTAGAACTCTAGGTAAATCGG | 8694 |
| hs132669985 | AGATTTATATTCCCAGTAAAAGG | 8695 |
| hs132669674 | CTACTGCTTAATCACCCATGGGG | 8696 |
| hs132669542 | AACATGAACCAGATGCACAGAGG | 8697 |
| hs132669647 | AATAACATAAAGAAGCTGCGTGG | 8698 |
| hs132669873 | TGGAAACCTAGAGTGCACAAAGG | 8699 |
| hs132669493 | ATAGACTTTATAAAACCTAGAGG | 8700 |
| hs132669226 | GGCCTAGGTTCACAGCCCAGCGG | 8701 |
| hs132669877 | CACAAAGGAGAAGACACTGGTGG | 8702 |
| hs132669704 | AGAGTCAAATAGACACTTGGGGG | 8703 |
| hs132669332 | TAATTCATAGTAATCTCCTGGGG | 8704 |
| hs132669518 | GCTCAAAAATTCTAGCAAGGTGG | 8705 |
| hs132669812 | AGGGTTTGGATAGATCCAGTCGG | 8706 |
| hs132669466 | AGATACGTTTTCATGTCCGGGGG | 8707 |
| hs132669451 | TGACTGGCAATAGAGAGCCGAGG | 8708 |
| hs132669209 | AAGCCCACTATCAGTCACCCCGG | 8709 |
| hs132669349 | GAAGAACCTAAGGATTGTGGAGG | 8710 |
| hs132669876 | GTGCACAAAGGAGAAGACACTGG | 8711 |
| hs132669902 | TCACTACCCCTAGATGTGAGAGG | 8712 |
| hs132669212 | TCTGAGAAATAATAGTTCCGGGG | 8713 |
| hs132669230 | TGGTTGTAGAGCTTACCGCTGGG | 8714 |
| hs132669579 | GAAGTCTTACACATTTGAGGGGG | 8715 |
| hs132669336 | ACACTTCTCTCTGTAAGTGGAGG | 8716 |
| hs132669696 | GAGGTGCTGAACAAATGAGTGGG | 8717 |
| hs132669288 | TTAGAGACACATTTACCATGAGG | 8718 |
| hs132669677 | ATGTTGATATCATACCCCATGGG | 8719 |
| hs132669738 | GCTGTGGAATACACTTGGGATGG | 8720 |
| hs132669396 | ATTAGCAAGGAAGTGAGTAGTGG | 8721 |
| hs132669302 | CAGAGCTCAAGAAATCTCTGTGG | 8722 |
| hs132669363 | CTATTAAATATGATCAAGAGTGG | 8723 |
| hs132669276 | ATAAAATTACAGGGAAACAGGGG | 8724 |
| hs132669920 | TTAAAAAAAAAAAAGGACCAAGG | 8725 |
| hs132669882 | TGGCAAGGACAGTAAAGAGGTGG | 8726 |
| hs132669859 | GGCAGACATCCCTTGAAGGGGGG | 8727 |
| hs132669904 | TTATTCGCCTGCTGTGATCGTGG | 8728 |
| hs132669683 | ATTTTTCTATCAAGTGACGTGGG | 8729 |
| hs132669387 | GACGCTTCCTTCCTTCACTGAGG | 8730 |
| hs132669580 | TGAAGTCTTACACATTTGAGGGG | 8731 |
| hs132669858 | TGGCAGACATCCCTTGAAGGGGG | 8732 |
| hs132669697 | GGAGGTGCTGAACAAATGAGTGG | 8733 |
| hs132669901 | ATTGTACCTCTCACATCTAGGGG | 8734 |
| hs132669756 | ATAAAAGCAACAGAATTGGAGGG | 8735 |
| hs132669273 | ACTCTTCATATAAAATTACAGGG | 8736 |
| hs132669832 | CATACATAGTCAAAGTGGCAAGG | 8737 |
| hs132669506 | AAAATACACCTAATAAAAGGAGG | 8738 |
| hs132669842 | TTATGTCAATCTCAGCTAGGTGG | 8739 |
| hs132669795 | ACTTGAATAATAGTAACTGTGGG | 8740 |
| hs132669607 | TTGGAGAATGAAGTGAGGCAAGG | 8741 |
| hs132669399 | CCATTGCAAGGCTGAACCAGAGG | 8742 |
| hs132669864 | GTTGCAATTTTAATTCCAGGAGG | 8743 |
| hs132669295 | TGATCGCACAAGGTGCAGGATGG | 8744 |
| hs132669473 | AACTCTGTTAGAATGTAGGAAGG | 8745 |
| hs132669739 | TAATGCTGTGGAATACACTTGGG | 8746 |
| hs132669326 | AAGATATTAACATTAATAGGAGG | 8747 |
| hs132669860 | GGATGTCTGCCATTAAGTGAAGG | 8748 |
| hs132669722 | AGAAACTAAAACTTCTGTGACGG | 8749 |
| hs132669871 | GAGCATTTAGCCTCCAGGAGAGG | 8750 |
| hs132669300 | GTGGACCCACAGATTAAGGTAGG | 8751 |
| hs132669307 | ATAGAACATGAAAGATTACTGGG | 8752 |
| hs132669913 | TATAGTGTGAAATTATGGAGGGG | 8753 |
| hs132669735 | AATACACTTGGGATGGTAGGAGG | 8754 |
| hs132669831 | GAACACATACATAGTCAAAGTGG | 8755 |
| hs132669590 | TACAGCTTCACAGATGCTGAGGG | 8756 |
| hs132669277 | TTTTATATGAAGAGTGTGAGTGG | 8757 |
| hs132669695 | ACGCAAACTTCACTGCACTTGGG | 8758 |
| hs132669210 | GTTCCGGGGTGACTGATAGTGGG | 8759 |
| hs132669805 | TTTCTCTAATCGCTAAACCAAGG | 8760 |
| hs132669643 | AAACTGTTCAGCTATATGGATGG | 8761 |
| hs132669596 | ATCTAGTTACTGAGGCAAGTAGG | 8762 |
| hs132669494 | AAAACTTACAAATTACCAAGAGG | 8763 |
| hs132669261 | GGATGCTGCATACTATTGGCAGG | 8764 |
| hs132669758 | GGAGGGAAAATACAATAGCAAGG | 8765 |
| hs132669224 | GTAATGAATTTAAAGTCCAAAGG | 8766 |
| hs132669376 | AGATTTATGTGACACTGGCAGGG | 8767 |
| hs132669428 | ATAGTGGTAGGTTAAAGAGCTGG | 8768 |
| hs132669319 | CATCTGGTTGGGGATTTTTGGGG | 8769 |
| hs132669378 | ACTGGCAGGGAAGTATCAAAGGG | 8770 |
| hs132669939 | AAAGGCACAGAAGCATGCACAGG | 8771 |
| hs132669715 | TTTTGGTTTATATCAGCAGGAGG | 8772 |
| hs132669592 | GTAGGTAAACTAGGCCAGTAGGG | 8773 |
| hs132669283 | ATAGCTGGTCCGAAGTATTGGGG | 8774 |
| hs132669395 | AGTTTTCTGGGTGATTAGCAAGG | 8775 |
| hs132669208 | GCATCTATTGGACATTGAGAAGG | 8776 |
| hs132669483 | ATGTCATTCCTCATAGACATTGG | 8777 |
| hs132669206 | TATGGAATGTTCGCATTTGGGGG | 8778 |
| hs132669482 | TCATTCCTCATAGACATTGGAGG | 8779 |
| hs132669801 | ACTAAAACTAAACAGAGAAGTGG | 8780 |
| hs132669427 | TTATTTCTGAGGATAGTGGTAGG | 8781 |
| hs132669667 | ACATTCGGTTAGGTTGCACATGG | 8782 |
| hs132669840 | AAGCTCTTGAATCTTCCCTAAGG | 8783 |
| hs132669586 | GAACTAGAGGACAATAGCGATGG | 8784 |
| hs132669609 | ATGAACACATTAGTTTTAGGGGG | 8785 |
| hs132669275 | TATAAAATTACAGGGAAACAGGG | 8786 |
| hs132669893 | TTAGTGGACCTGGTAAACACTGG | 8787 |
| hs132669622 | TAATAACTATGGCAATTAGTGGG | 8788 |
| hs132669569 | TGTTGCATGGCAATAATGAATGG | 8789 |
| hs132669467 | CAGATACGTTTTCATGTCCGGGG | 8790 |
| hs132669825 | AAAACGTGATTATGAAAAGAGGG | 8791 |
| hs132669388 | TCACAGGCCTCAGTGAAGGAAGG | 8792 |
| hs132669834 | AAAAGTCTATGTGATAGAAGTGG | 8793 |
| hs132669373 | TACCTTAAGTAGACTAGCAGTGG | 8794 |
| hs132669423 | TTTTTAACATAACCTTGCCAAGG | 8795 |
| hs132669630 | TTTCTGAACTATTTACTGTGAGG | 8796 |
| hs132669720 | TAAAACTTCTGTGACGGTAAGGG | 8797 |
| hs132669655 | GCAGATGTCATCAGGAGCCAAGG | 8798 |
| hs132669221 | CTATTTTCTATATCTTTGTGGGG | 8799 |
| hs132669570 | GGCAATAATGAATGGTCCATAGG | 8800 |
| hs132669299 | CATAGCCTACCTTAATCTGTGGG | 8801 |
| hs132669625 | ACCCTGCAACCTGATCTTGGAGG | 8802 |
| hs132669894 | TAGTGGACCTGGTAAACACTGGG | 8803 |
| hs132669600 | CTGCAAAGCCTGACACTGCATGG | 8804 |
| hs132669432 | GCACAGTCTTGGGTCACACAGGG | 8805 |
| hs132669218 | ACAAAGATATAGAAAATAGTGGG | 8806 |
| hs132669685 | AACAGAAGATATCTAAGTAGAGG | 8807 |
| hs132669744 | ATTGGCTTACTCCTCTTAAGGGG | 8808 |
| hs132669271 | TGGTATGTAACATTACATACAGG | 8809 |
| hs132669285 | TGATAACAACCCCAATACTTCGG | 8810 |
| hs132669397 | GCTAATCACCCAGAAAACTCTGG | 8811 |
| hs132669398 | GTGAGTAGTGGTCCATTGCAAGG | 8812 |
| hs132669263 | TATCCTCATACACTAATGCCAGG | 8813 |
| hs132669619 | GATGTCAGCATACTTCGTGTTGG | 8814 |
| hs132669338 | ACTGCCAACTGCCAAAGAATGGG | 8815 |
| hs132669389 | AAAGAGCATCAGTAAAGTGCAGG | 8816 |
| hs132669293 | CAGTGCAGCTTGATCGCACAAGG | 8817 |
| hs132669589 | TGAAGCTGTAGAAACCCTACTGG | 8818 |
| hs132669591 | CTACAGCTTCACAGATGCTGAGG | 8819 |
| hs132669422 | ATATTAGTTGTAAAATACACTGG | 8820 |
| hs132669767 | TTGCAGTTATTTTTTCACAATGG | 8821 |
| hs132669462 | GAGAGCTGTCATCCCACCCCCGG | 8822 |
| hs132669678 | TATGTTGATATCATACCCCATGG | 8823 |
| hs132669322 | TTTTGGGGTAGGGACCAGAATGG | 8824 |
| hs132669673 | GCTACTGCTTAATCACCCATGGG | 8825 |
| hs132669535 | TGCCACTTGTGAGGACCCAAGGG | 8826 |
| hs132669324 | GGGTAGGGACCAGAATGGCTGGG | 8827 |
| hs132669684 | TATTTTTCTATCAAGTGACGTGG | 8828 |
| hs132669420 | GAGTGGGGAGGTTAACCTCTGGG | 8829 |
| hs132669238 | AATTACACAACTTTATGTTGAGG | 8830 |
| hs132669841 | TTTTTATGTCAATCTCAGCTAGG | 8831 |
| hs132669645 | TAATATTTTTCAACTATCCGAGG | 8832 |
| hs132669201 | GTAATAAGTTGCTTTCAAAGAGG | 8833 |
| hs132669845 | TTAGCATTTCCATTCTCTAGTGG | 8834 |
| hs132669350 | ATCACACCTCCACAATCCTTAGG | 8835 |
| hs132669593 | AGTAGGTAAACTAGGCCAGTAGG | 8836 |
| hs132669434 | TGCACAGTCTTGGGTCACACAGG | 8837 |
| hs132669461 | TTTTGCCTATACCTACTGTCAGG | 8838 |
| hs132669914 | TTATAGTGTGAAATTATGGAGGG | 8839 |
| hs132669670 | CTATTGGACTAATAAGAAGTAGG | 8840 |
| hs132669289 | TCTGATTGCAATGTTCCTCATGG | 8841 |
| hs132669856 | AATGGCAGACATCCCTTGAAGGG | 8842 |
| hs132669627 | ACCTCCAAGATCAGGTTGCAGGG | 8843 |
| hs132669809 | AGGAAATAATGGCTTCCGACTGG | 8844 |
| hs132669560 | ATGAGTCTCGAAGTTTATTGTGG | 8845 |
| hs132669260 | GTGTGGATGCTGCATACTATTGG | 8846 |
| hs132669503 | GCCAGTAACTGTGAATTCTAGGG | 8847 |
| hs132669527 | GTGATAGTTTGAATAAAAGCCGG | 8848 |
| hs132669915 | ATTATAGTGTGAAATTATGGAGG | 8849 |
| hs132669382 | CTTTACTGAAGGGCATACTTGGG | 8850 |
| hs132669789 | AATCCTCTCCAGTTTGCCTAAGG | 8851 |
| hs132669814 | TTCTCTCTCTACCTAAGACAGGG | 8852 |
| hs132669386 | TGAAATAACTAGTAATGAAGAGG | 8853 |
| hs132669903 | ATCATCACCACGATCACAGCAGG | 8854 |
| hs132669548 | TTTTTATTTGTAATTTCTGGGGG | 8855 |
| hs132669479 | TTTGTCCTCCAATGTCTATGAGG | 8856 |
| hs132669400 | AGAGGAAGAAGCAAAATTGAAGG | 8857 |
| hs132669740 | TTAATGCTGTGGAATACACTTGG | 8858 |
| hs132669608 | TGCCTCACTTCATTCTCCAATGG | 8859 |
| hs132669425 | AGTATTATAAATCTTTACCTTGG | 8860 |
| hs132669924 | AATGTATTCAAATCCACAGTTGG | 8861 |
| hs132669357 | GCAAATAGTAGTCCATATGCTGG | 8862 |
| hs132669613 | GGAAAGAAGGTGCTGTAAAGAGG | 8863 |
| hs132669846 | TAGCATTTCCATTCTCTAGTGGG | 8864 |
| hs132669367 | ATTTTGTCCTATAGGAAGGAAGG | 8865 |
| hs132669705 | ATTTGACTCTGGAAATAAGATGG | 8866 |
| hs132669806 | TAATCGCTAAACCAAGGCTTAGG | 8867 |
| hs132669779 | CTGAGGCAATTAGAAAAGGCTGG | 8868 |
| hs132669543 | CCAGATGCACAGAGGTAACATGG | 8869 |
| hs132669287 | TTATTGTAAACACTCTCAAAAGG | 8870 |
| hs132669621 | GATACATGACATGCTCTCCTTGG | 8871 |
| hs132669930 | ATATTGACTTAATAAAAAAGAGG | 8872 |
| hs132669384 | CCCTTCCTTACTTTACTGAAGGG | 8873 |
| hs132669818 | TCCAAGTTGAAGCTAATCATTGG | 8874 |
| hs132669750 | GTTTTGTAATTGACCAGTATCGG | 8875 |
| hs132669337 | GACTGCCAACTGCCAAAGAATGG | 8876 |
| hs132669290 | AGATGAGATATCCATTTAAGTGG | 8877 |
| hs132669445 | CTTCAAGTCGTCATTTAGACGGG | 8878 |
| hs132669364 | CCCTAAACCTTCCTTCCTATAGG | 8879 |
| hs132669617 | ATACTTCGTGTTGGGAAAGAAGG | 8880 |
| hs132669838 | GAATAATTATTAAGAAGGCAAGG | 8881 |
| hs132669932 | TTTGAATAATGTTTTTCAAGAGG | 8882 |
| hs132669616 | GTATGCTGACATCTTGGGCTAGG | 8883 |
| hs132669444 | CCTTCAAGTCGTCATTTAGACGG | 8884 |
| hs132669379 | GTATGCCCTTCAGTAAAGTAAGG | 8885 |
| hs132669348 | CTGTTTTCAAACTTGTTCTGAGG | 8886 |
| hs132669891 | TAATAGATGGAGTAAATTAGTGG | 8887 |
| hs132669488 | CTATTAGGACCATCAAATACAGG | 8888 |
| hs132669475 | TTGGTAATCTAATTATCTGTCGG | 8889 |
| hs132669453 | GCAATAGAGAGCCGAGGGAGTGG | 8890 |
| hs132669294 | AGCTTGATCGCACAAGGTGCAGG | 8891 |
| hs132669861 | TCAAAAATTCCCCCCTTCAAGGG | 8892 |
| hs132669909 | ATAAGGTACATGTAAGATAGAGG | 8893 |
| hs132669749 | CATTTAAATCATATAAACATTGG | 8894 |
| hs132669745 | TTTTGTTAGAACCCCTTAAGAGG | 8895 |
| hs132669852 | ATCCCCATCCCACTAGAGAATGG | 8896 |
| hs132669672 | TGCTACTGCTTAATCACCCATGG | 8897 |
| hs132669524 | ATTGAGGTAATGTTTAAGTCCGG | 8898 |
| hs132669539 | CCAGACAACATGACTTGTTAGGG | 8899 |
| hs132669633 | TCATTTCTAGTTGTTGGATGGGG | 8900 |
| hs132669497 | TGTGAGCTCTTTAGGAAGACAGG | 8901 |
| hs132669231 | CTGGTTGTAGAGCTTACCGCTGG | 8902 |
| hs132669917 | GCATATCTAATGAGTTTCCTTGG | 8903 |
| hs132669225 | AATTTAAAGTCCAAAGGCCTAGG | 8904 |
| hs132669284 | CCAATACTTCGGACCAGCTATGG | 8905 |
| hs132669731 | TGGAAGTGCAAGAAGACAACTGG | 8906 |
| hs132669639 | TTTCTCACATTCCATTAAGTAGG | 8907 |
| hs132669470 | ATGAACATAAAGGAATTCAATGG | 8908 |
| hs132669668 | TGATAACGTGACATTCGGTTAGG | 8909 |
| hs132669402 | CCTCTGGTTCAGCCTTGCAATGG | 8910 |
| hs132669321 | GGTTGGGGATTTTTGGGGTAGGG | 8911 |
| hs132669457 | TTATTCTAATTCCACTCCCTCGG | 8912 |
| hs132669919 | AACTCATTAGATATGCTAGAAGG | 8913 |
| hs132669669 | ATAGATGATAACGTGACATTCGG | 8914 |
| hs132669335 | CTGTAAGTGGAGGTGAAGTCAGG | 8915 |
| hs132669810 | ATTATTTCCTGCCTAAGCCTTGG | 8916 |
| hs132669525 | TTATTCAAACTATCACAGATAGG | 8917 |
| hs132669660 | ATAGTTTTGATATTTCTGATTGG | 8918 |
| hs132669796 | CTATTATTCAAGTTGCATCTAGG | 8919 |
| hs132669237 | TTATCCCATGTCCCTGATGCTGG | 8920 |
| hs132669941 | AGTGACATTTTAATGTTTGGAGG | 8921 |
| hs132669353 | TCAGAATCCTTTCCAACAATGGG | 8922 |
| hs132669534 | CCTCACAAGTGGCAAATACCTGG | 8923 |
| hs132669484 | ATTGCTAGATTGCTAAACTTAGG | 8924 |
| hs132669836 | CAAAAGCTATACCAGCTAGAAGG | 8925 |
| hs132669829 | GTTCAGCAAAGCTTACATAATGG | 8926 |
| hs132669455 | AATTAGAATAAAGTAGTATAGGG | 8927 |
| hs132669657 | GATATATTCATATTTGTCCTTGG | 8928 |
| hs132669472 | CAGAAACTCTGTTAGAATGTAGG | 8929 |
| hs132669691 | ATTCGTTGCAGTAAATACTTTGG | 8930 |
| hs132669753 | TCTCCAGTCACAAATGAGTTTGG | 8931 |
| hs132669723 | ACAATTATCAGAAAAGTTGATGG | 8932 |
| hs132669889 | ATCTTAATATCGATAATAGATGG | 8933 |
| hs132669585 | TCTCATGCTAACTTTCTGAATGG | 8934 |
| hs132669815 | TTTCTCTCTCTACCTAAGACAGG | 8935 |
| hs132669804 | TAGAAATTATAGCATAGCTTTGG | 8936 |
| hs132669565 | TATGGTGTATCATTAAATATAGG | 8937 |
| hs132669650 | AATCAGGAAGATTGATTTGAGGG | 8938 |
| hs132669573 | TGATAAACGTTATAAATCTTGGG | 8939 |
| hs132669641 | ATATGGATGGAGGACAAAAATGG | 8940 |
| hs132669635 | CTTCATTTCTAGTTGTTGGATGG | 8941 |
| hs132669248 | CAGAAGATACTCGATCAATATGG | 8942 |
| hs132669786 | CCAGTTTGCCTAAGGAATAAGGG | 8943 |
| hs132669816 | AGATTACTAGTAAACAAAAATGG | 8944 |
| hs132669365 | CCTATAGGAAGGAAGGTTTAGGG | 8945 |
| hs132669687 | TGTGAAATAAAATCACAATTAGG | 8946 |
| hs132669250 | ATATGGAAACACTAATAATATGG | 8947 |
| hs132669391 | GTAAAGTGCAGGTGAATTATAGG | 8948 |
| hs132669820 | CATTCTCATGACCATGATAATGG | 8949 |
| hs132669370 | AATTGGATGCGAAGTATAATTGG | 8950 |
| hs132669200 | GCTTTCAAAGAGGTCATAATAGG | 8951 |
| hs132669830 | TTATCACATTAGAATTTAATAGG | 8952 |
| hs132669408 | CCAAAGATGGTTTAAATAAAGGG | 8953 |
| hs132669956 | TTAAGAAGACTAACAAGTTTTGG | 8954 |
| hs132669514 | AAAATTGTACTGATGAAAATTGG | 8955 |
| hs132669508 | ATACATTACCTCCTTTTATTAGG | 8956 |
| hs132669291 | AGTTTTTGCTTCCACTTAAATGG | 8957 |
| hs132669107 | GGAGTGTAAAGCGAAATCCAGGG | 8958 |
| hs132669142 | AGATCTTAGAGGACTCCTGGGGG | 8959 |
| hs132668681 | AGAAATACAATTATTCACAGAGG | 8960 |
| hs132668554 | TAAACACAGGAATAAATTAGGGG | 8961 |
| hs132668867 | TCTACAGTTTTGGCACACTGAGG | 8962 |
| hs132668978 | AAACCCTCATTATAGCACATGGG | 8963 |
| hs132668700 | ATACTTAGGAAATAAAGACATGG | 8964 |
| hs132668561 | TTATGGGATCTGGAGCACCCAGG | 8965 |
| hs132668974 | TGTACTATACTGAATGCTGGAGG | 8966 |
| hs132669081 | AATAAATAAATAATGCCTGGAGG | 8967 |
| hs132669141 | TAGATCTTAGAGGACTCCTGGGG | 8968 |
| hs132669030 | AAGCAATTGATCCTATATGGTGG | 8969 |
| hs132668775 | ACTCCTAAGCTACCAAATGGGGG | 8970 |
| hs132668550 | TATTTTGTAATGATGAACAGGGG | 8971 |
| hs132668718 | TGGGTATTCAATGAAGTCGGAGG | 8972 |
| hs132668551 | TGATGAACAGGGGTAAACACAGG | 8973 |
| hs132668663 | TCATAGATGAATTTATACAACGG | 8974 |
| hs132668981 | TATAGAGTATATATTCACATCGG | 8975 |
| hs132668653 | AATAAAGGCCTTGATCATTGGGG | 8976 |
| hs132669006 | TGTGATATATTTTTTCACAGGGG | 8977 |
| hs132668980 | TAGGTGACTGAGTTTCTAAGAGG | 8978 |
| hs132668906 | TAACAAGCATCATATTCCAAGGG | 8979 |
| hs132668971 | GGATTTAAGCCTGATCATGGAGG | 8980 |
| hs132668996 | GTGGCATGATGCTATTGAGGAGG | 8981 |
| hs132668543 | TGACAGCCTCACTTAATAGGGGG | 8982 |
| hs132669036 | TGTTGAAATGAAGATAGCAAAGG | 8983 |
| hs132668896 | GAAGACACTGACCTCTCCAATGG | 8984 |
| hs132669011 | CCCAAGAGTGAGTCTTTCTGGGG | 8985 |
| hs132668970 | GATTTAAGCCTGATCATGGAGGG | 8986 |
| hs132668633 | TGTACTCACCGTAAGTCCAGAGG | 8987 |
| hs132668924 | CATGGCTAACTTTCTGACAAGGG | 8988 |
| hs132668536 | AGGGTTCAGTTGAGACCAAGTGG | 8989 |
| hs132669121 | TAACTTCCAGTGTAGGCCAAGGG | 8990 |
| hs132668807 | TCTGTGATAACCATCAGATGAGG | 8991 |
| hs132669127 | AATGTGCCACATCGGAAAAGCGG | 8992 |
| hs132668916 | ACACCAGAGATAACATCACCAGG | 8993 |
| hs132669186 | ACACAGTAACACAGATGACATGG | 8994 |
| hs132668819 | CTATATGCAGAATGTATGGGTGG | 8995 |
| hs132669177 | TTGTGGATGAGAACAACGCAGGG | 8996 |
| hs132669128 | GCCACATCGGAAAAGCGGTACGG | 8997 |
| hs132668632 | GTACTCACCGTAAGTCCAGAGGG | 8998 |
| hs132669017 | CAATGTCTATGAAGGATGAGTGG | 8999 |
| hs132669166 | AAAAATGCTTTCTGGCAGCGAGG | 9000 |
| hs132669178 | TTTGTGGATGAGAACAACGCAGG | 9001 |
| hs132668804 | GTTCAAACTACCTCATCTGATGG | 9002 |
| hs132668568 | CAATAATAAATAGTGTAACAGGG | 9003 |
| hs132669159 | AGGCTCCTGCTCAATTCATGAGG | 9004 |
| hs132669053 | AGTTTGGTGATGCAGCAGATGGG | 9005 |
| hs132669110 | TTCTGATGTTCAGTGTTACGTGG | 9006 |
| hs132668549 | TTATTTTGTAATGATGAACAGGG | 9007 |
| hs132668542 | TCATTTCCCCCTATTAAGTGAGG | 9008 |
| hs132668724 | GCTGTTCATCATGAATTGGAAGG | 9009 |
| hs132669020 | TCCTTCATAGACATTGCTGTAGG | 9010 |
| hs132668528 | GTTTTCCCCAAACCCCCACATGG | 9011 |
| hs132668977 | GCACCCATGTGCTATAATGAGGG | 9012 |
| hs132668859 | CAGTGCTTAGATTTGGTAGGTGG | 9013 |
| hs132668808 | GCATGGGCAGACATATGTCATGG | 9014 |
| hs132668998 | AAGGATTTCCCTCGAAGGAGTGG | 9015 |
| hs132669126 | GAGAAGCAAATGTGCCACATCGG | 9016 |
| hs132668984 | TGTGCATAGATGCATAACCTTGG | 9017 |
| hs132669101 | AGCTAAGTACGGATAACCTAGGG | 9018 |
| hs132668582 | TATTCAAACTCTATGGCAGGTGG | 9019 |
| hs132668557 | AGGAGGCTGCTCTAGAACCAAGG | 9020 |
| hs132668894 | GAGGTCAGTGTCTTCTCATGAGG | 9021 |
| hs132669144 | CTGGGGGTCAATAATAGTAAGGG | 9022 |
| hs132669145 | GGGTCAATAATAGTAAGGGCTGG | 9023 |
| hs132669114 | ACGTAACACTGAACATCAGAAGG | 9024 |
| hs132668719 | TGTTGGGTATTCAATGAAGTCGG | 9025 |
| hs132668530 | TAATCTTTGTGCAATCAGGAAGG | 9026 |
| hs132668760 | CATATCATTCAGGCCAGCAAGGG | 9027 |
| hs132668938 | ACTCACTCTCATATTTAGCAAGG | 9028 |
| hs132669079 | AAGGGCATAAAGAGGCCAGAAGG | 9029 |
| hs132668744 | GGATTTCTTGGGAAATGATGGGG | 9030 |
| hs132668715 | CCATAGCTGAGTCTATGCTATGG | 9031 |
| hs132668941 | GACAGTGCCATAGGATCAAGTGG | 9032 |
| hs132668757 | TAGAGCCTAGCCCAGTGTAGAGG | 9033 |
| hs132669102 | TAGCTAAGTACGGATAACCTAGG | 9034 |
| hs132668576 | GCACGTTTGCCAGAAAGTAACGG | 9035 |
| hs132669120 | GTAACTTCCAGTGTAGGCCAAGG | 9036 |
| hs132669008 | TTTTTTCACAGGGGAATGAGAGG | 9037 |
| hs132668544 | GTGACAGCCTCACTTAATAGGGG | 9038 |
| hs132668587 | TGGAAGGGAACTTATTAGGATGG | 9039 |
| hs132668805 | TTATCACAGAAAAATTTGCATGG | 9040 |
| hs132668982 | CTATATTCTGACTTCAGTGAAGG | 9041 |
| hs132668710 | GGGTATCCTGTAAATTCGATGGG | 9042 |
| hs132668829 | TTATTGGGAGAGAGTTGGGGTGG | 9043 |
| hs132668995 | GCATCATGCCACTCCTTCGAGGG | 9044 |
| hs132668593 | TATAAGCTCTGGAATGTCAAAGG | 9045 |
| hs132668930 | TTAATGGTTATGATGGTAAGGGG | 9046 |
| hs132668781 | AATGCTTCTCAATAGCCTGAAGG | 9047 |
| hs132668674 | AATGGTAATGAAATTTAACTAGG | 9048 |
| hs132668961 | AGAGCAGAGAAACAGGAGGCCGG | 9049 |
| hs132668621 | ATTCTTGCCATATTGATCAAGGG | 9050 |
| hs132669104 | AATGTTAGCATAGCTAAGTACGG | 9051 |
| hs132668713 | CCATAGCATAGACTCAGCTATGG | 9052 |
| hs132668806 | TATCACAGAAAAATTTGCATGGG | 9053 |
| hs132668725 | GCGTTCTGTGTTGTTTCTAGGGG | 9054 |
| hs132668642 | TAGCTGTAAGACAAAAGCTATGG | 9055 |
| hs132668832 | TGATTATCCTCCTGCTCAGACGG | 9056 |
| hs132668875 | GTTATGAACAGAATTTCTAGTGG | 9057 |
| hs132668787 | CTACATATGGAAAAGGACATAGG | 9058 |
| hs132668617 | AAATCAGAACAACTAAGTAATGG | 9059 |
| hs132668794 | AGTCAGAATGTATTAGATGAGGG | 9060 |
| hs132668816 | GCATGGATTGTAGAAAGAGTAGG | 9061 |
| hs132668539 | GCCTTTCTAACTTACAAATGTGG | 9062 |
| hs132668862 | AATCTCAGTGTAAGAATGCAAGG | 9063 |
| hs132669044 | TTAAATTCTGCTCAGATCAGAGG | 9064 |
| hs132668815 | GATTGTAGAAAGAGTAGGAAAGG | 9065 |
| hs132668571 | TTTGTTACATAACACAAGAGTGG | 9066 |
| hs132668590 | TATAGTGTTCTAAAATGTTGAGG | 9067 |
| hs132668523 | GTCATAGCAGAAAATTGGCTTGG | 9068 |
| hs132668893 | TCCTTTAAACACCATTGGAGAGG | 9069 |
| hs132668878 | TCAACCTTATATTTTAACACGGG | 9070 |
| hs132668736 | TTAATTCAAGACATCTGGACAGG | 9071 |
| hs132668637 | GCAAATCTACTCCAGCACTCTGG | 9072 |
| hs132668748 | CCTTCTACCACCTCTACACTGGG | 9073 |
| hs132669097 | ACTTCCTTCATAGCTGCCCTAGG | 9074 |
| hs132668836 | ATATAGGCTTATGATAAGAATGG | 9075 |
| hs132668844 | AAACATAGAGTAAGCAGTAGTGG | 9076 |
| hs132668907 | TTAACAAGCATCATATTCCAAGG | 9077 |
| hs132668594 | ATAAAACTCTTTTTGCCATGAGG | 9078 |
| hs132668527 | AATGTTAAGAAAGGTACAGCAGG | 9079 |
| hs132668864 | ATAAATAGTGATGCTCAAAAGGG | 9080 |
| hs132668707 | ATAGAAAACAAAAACAAGGATGG | 9081 |
| hs132668750 | GTAGAAGGAATGGGGCATAGAGG | 9082 |
| hs132668630 | GTACAAGGAAATAAAGGAGTGGG | 9083 |
| hs132669119 | CTGCATGTAACTTCCAGTGTAGG | 9084 |
| hs132668589 | GCTTCGAAAATAAGAAGCCTAGG | 9085 |
| hs132668703 | CATGGAATTATGTAAGGCTAGGG | 9086 |
| hs132668902 | GTAAAATAAGAATATGGGCATGG | 9087 |
| hs132668613 | AAGAAGTGTAAGAAAAACATTGG | 9088 |
| hs132669056 | AGATGGGAGTTGCTTTTGTGGGG | 9089 |
| hs132669163 | TTTCTTATCCAAAATTATGAGGG | 9090 |
| hs132668934 | CTTTCATTAAGCACTTATGAGGG | 9091 |
| hs132668858 | GTACAGTGCTTAGATTTGGTAGG | 9092 |
| hs132668676 | TGACATTTGACAACAAACATTGG | 9093 |
| hs132668697 | ACACATGTAAAATAATACTTAGG | 9094 |
| hs132668625 | AATCTAACACAAGAACCCTCTGG | 9095 |
| hs132668534 | CTTGGTCTCAACTGAACCCTAGG | 9096 |
| hs132668721 | AGTACCGGCAATTAGCTGTTGGG | 9097 |
| hs132668782 | ATGCTTCTCAATAGCCTGAAGGG | 9098 |
| hs132668991 | TTACTATTAATGAAAGCTCAAGG | 9099 |
| hs132669019 | GATGAGTGGTTTCTCCCAAATGG | 9100 |
| hs132669000 | TGATGTTGACAAAGAATGAAAGG | 9101 |
| hs132668801 | TACACTACATTAATAAAAGTAGG | 9102 |
| hs132668776 | CCATTTGGTAGCTTAGGAGTTGG | 9103 |
| hs132668931 | TTTAATGGTTATGATGGTAAGGG | 9104 |
| hs132668679 | TTAATGTTACATCTCACTGTTGG | 9105 |
| hs132669041 | CTCTCTCTCTCTCAACTGGAGGG | 9106 |
| hs132668796 | CTAATAGACATCATAAGTGCAGG | 9107 |
| hs132669133 | TTTCCTGATGGCATAGCAGACGG | 9108 |
| hs132668797 | ACTTATGATGTCTATTAGAAAGG | 9109 |
| hs132668770 | GAATATCAAAAGCTTTTCTAGGG | 9110 |
| hs132668526 | GAGAAGACAAATGTTAAGAAAGG | 9111 |
| hs132669042 | GCTCTCTCTCTCTCAACTGGAGG | 9112 |
| hs132668960 | AGGCCGGAAAACATGTTAAGAGG | 9113 |
| hs132669140 | CTAGATCTTAGAGGACTCCTGGG | 9114 |
| hs132668646 | TATGAACTATGCAACGTTCAGGG | 9115 |
| hs132669052 | AAGTTTGGTGATGCAGCAGATGG | 9116 |
| hs132668936 | CACTTATGAGGGCAAAAGACAGG | 9117 |
| hs132668942 | GTTAAGTTTGACAGTGCCATAGG | 9118 |
| hs132669039 | GCTTCTGTATATTCTGTGACAGG | 9119 |
| hs132668840 | AGAGGAAAAAGAGACTATAAGGG | 9120 |
| hs132668846 | ATCTAGACATTCCCAATCCTGGG | 9121 |
| hs132668842 | TTAAAGAACTACAGGAGTAATGG | 9122 |
| hs132669010 | CCCCAGAAAGACTCACTCTTGGG | 9123 |
| hs132668810 | GACAGTCCCTAATCACGAAATGG | 9124 |
| hs132668615 | TAAGAGTTGGATTCATTCCATGG | 9125 |
| hs132669082 | TTTATTTAAAGGGCATAAAGAGG | 9126 |
| hs132668573 | AAGAGTGGAGAAACAAGTTTGGG | 9127 |
| hs132669183 | TCATCTGTGTTACTGTGTATAGG | 9128 |
| hs132668913 | CTATTAAGATATTTGCATATGGG | 9129 |
| hs132668567 | ATAAATAGTGTAACAGGGTAAGG | 9130 |
| hs132668874 | TCATAACAGAAATATTAAACAGG | 9131 |
| hs132669073 | GTGCTCAAATAGTTAAAAGTAGG | 9132 |
| hs132668788 | CTATGTCCTTTTCCATATGTAGG | 9133 |
| hs132668717 | AATACCCAACAGCTAATTGCCGG | 9134 |
| hs132668813 | AGGAAAGGCAAGAGAATTGAGGG | 9135 |
| hs132668752 | GTAGAGGTGGTAGAAGGAATGGG | 9136 |
| hs132668854 | GCTCTAGTCTCTAGGATCTCAGG | 9137 |
| hs132668779 | AGTCTTTCAGAACCCCCATTTGG | 9138 |
| hs132669181 | TTAAAAAATACAGCATAGCTAGG | 9139 |
| hs132668609 | GTGGCCAGTGTATCATAAGTTGG | 9140 |
| hs132668517 | TGAAATGATTTGCTCTCAAATGG | 9141 |
| hs132669025 | GTAACCAAGAAAAGTTCAATTGG | 9142 |
| hs132668914 | GGGAATGCAACAGAGATAGAAGG | 9143 |
| hs132669029 | CTTTACCTTACCTAATTGCCAGG | 9144 |
| hs132668668 | CATTTTTGTGTACTTCTCTAGGG | 9145 |
| hs132668772 | CCAACTCCTAAGCTACCAAATGG | 9146 |
| hs132669080 | TTAAATAAATAAATAATGCCTGG | 9147 |
| hs132669174 | CCTGGAAGACAGACTTCATAGGG | 9148 |
| hs132668714 | GACTCAGCTATGGAAAGAACAGG | 9149 |
| hs132669131 | GAGCACCTGAAGAAGAAGTTAGG | 9150 |
| hs132668917 | TTACCTGGTGATGTTATCTCTGG | 9151 |
| hs132669089 | AATTTCATAGTCATTAGAAATGG | 9152 |
| hs132668985 | GTCTAAATCTCTCAAAAGAATGG | 9153 |
| hs132668616 | TTAAAAGACTCAATAAGAGTTGG | 9154 |
| hs132668921 | GATAATTTCAATTAAAACGTTGG | 9155 |
| hs132668847 | ATTGGGAATGTCTAGATAGAAGG | 9156 |
| hs132668821 | GTAACTATATGCAGAATGTATGG | 9157 |
| hs132669105 | TCTTTTGGAGCTCTTCTCCCTGG | 9158 |
| hs132668958 | TGACAACAAAAATTGTAAACAGG | 9159 |
| hs132668698 | TTTACATGTGTTCTAAGAAAGGG | 9160 |
| hs132669143 | CCTGGGGGTCAATAATAGTAAGG | 9161 |
| hs132668691 | GGCTCTTGTCCATGTCAAGTTGG | 9162 |
| hs132668892 | TTTTTTCCTTTAAACACCATTGG | 9163 |
| hs132668891 | AACTCAGTAAATTAGAAATAGGG | 9164 |
| hs132668843 | CCTGTAGTTCTTTAAAATAGAGG | 9165 |
| hs132668565 | GTAACAGGGTAAGGTCTTATGGG | 9166 |
| hs132668908 | ATTATTTTGACATAAATGATTGG | 9167 |
| hs132668612 | TTACATGTAAAAATGAAAACTGG | 9168 |
| hs132668946 | GGGCAATTGGGACTTGAGATTGG | 9169 |
| hs132668537 | ATTATTCTGGTTTTATCCTAGGG | 9170 |
| hs132668519 | GTTTTAGGTTAATAAAAAATGGG | 9171 |
| hs132668920 | ATAATTTCAATTAAAACGTTGGG | 9172 |
| hs132668973 | TACAAGACATCGTAAATGATTGG | 9173 |
| hs132669116 | ACTTCAACTTCCAAAAGTATTGG | 9174 |
| hs132668592 | TGGAATGTCAAAGGCATGATAGG | 9175 |
| hs132668754 | CCCAGTGTAGAGGTGGTAGAAGG | 9176 |
| hs132668624 | CATCTCACAACTCTCTTTAGTGG | 9177 |
| hs132668940 | GGATCAAGTGGTGCTTTTAGTGG | 9178 |
| hs132668580 | TCTTAGGTATTCAAACTCTATGG | 9179 |
| hs132668591 | TGACATTCCAGAGCTTATAATGG | 9180 |
| hs132668857 | GCTTGTACAGTGCTTAGATTTGG | 9181 |
| hs132668541 | GCCACATTTGTAAGTTAGAAAGG | 9182 |
| hs132668545 | TGTGACAGCCTCACTTAATAGGG | 9183 |
| hs132668898 | TCAAGACTCTGTCATCTTAGAGG | 9184 |
| hs132668643 | CATTTGAAGTCTGAGAAAATAGG | 9185 |
| hs132668784 | GTATTTATTGAGTCCCCTTCAGG | 9186 |
| hs132668683 | TAAATAAGTCTCTAGTTAAAGGG | 9187 |
| hs132668871 | GGTAGTACTCTCTTATTCATTGG | 9188 |
| hs132668553 | GTAAACACAGGAATAAATTAGGG | 9189 |
| hs132668623 | ATATAAACCCTTGATCAATATGG | 9190 |
| hs132668905 | CTTGTTAATAGAATTCTTATGGG | 9191 |
| hs132668524 | TGAGCGTCATAGCAGAAAATTGG | 9192 |
| hs132668654 | TTATTCATGGATTCCTAGTTTGG | 9193 |
| hs132669137 | TCTTAAATGCTAGATCTTAGAGG | 9194 |
| hs132668766 | TTTCCATGAAGATCTATTTACGG | 9195 |
| hs132668945 | AGATTGGTAATTAAGAAAATAGG | 9196 |
| hs132668965 | GTTTAATTTCTCATGAATAATGG | 9197 |
| hs132668711 | GCTTCTCCCATCGAATTTACAGG | 9198 |
| hs132669118 | TTAAATGTGTCCAATACTTTTGG | 9199 |
| hs132668664 | TATACAACGGAGATTTATATTGG | 9200 |
| hs132668803 | TTAATGTAGTGTAAATAAATTGG | 9201 |
| hs132668596 | TTTTGCCATGAGGCCAAAAATGG | 9202 |
| hs132668937 | AGTGCTTAATGAAAGTTTAATGG | 9203 |
| hs132668574 | GGACATTTCCCGTTACTTTCTGG | 9204 |
| hs132668096 | GTATAACATATGGTACAGGGCGG | 9205 |
| hs132668025 | CCATGCTCAGATCCACAACGGGG | 9206 |
| hs132668325 | AGATGTATTGAAGGTCTGTGTGG | 9207 |
| hs132668193 | CCCACCATACAAAATTACAGGGG | 9208 |
| hs132668355 | GGAATGCTTAGAAAGAGATGTGG | 9209 |
| hs132668065 | CACACAATGCATCAGCAACGGGG | 9210 |
| hs132668294 | CAATAAACAGTATCACGCAGAGG | 9211 |
| hs132667922 | ATGTGTATTAACCCACCTAGAGG | 9212 |
| hs132668104 | TTTAGCTGTCACTTCCAAAGGGG | 9213 |
| hs132668151 | CAAGGAGCTCAGAGTCTGCGAGG | 9214 |
| hs132668243 | AGTCGTCTGTTTAAACCCGGTGG | 9215 |
| hs132668026 | TGCTCAGATCCACAACGGGGAGG | 9216 |
| hs132668225 | GTGTAACATGAGTATCACACAGG | 9217 |
| hs132668463 | GAAGGCAGTAGCCATTAAAGGGG | 9218 |
| hs132668395 | TCTGTTGAAATGCCATCCAGAGG | 9219 |
| hs132668274 | AGGTTTATAGCTATGTCCTGAGG | 9220 |
| hs132668505 | GATACAATAGTAACTAACTGCGG | 9221 |
| hs132668095 | ACTGTATAACATATGGTACAGGG | 9222 |
| hs132668058 | CAGGCGTATATCCACTAAGGCGG | 9223 |
| hs132668349 | AACGCAACAGATGATTCCTGAGG | 9224 |
| hs132668176 | GAGTTGGCATGAGACATAGAGGG | 9225 |
| hs132668043 | GAAAATCTGACAAGCCTGGGAGG | 9226 |
| hs132668036 | ATAGCAACTCAACCAAACAAAGG | 9227 |
| hs132668479 | CAGTCCGTAGAACTCACAGATGG | 9228 |
| hs132668401 | TGTGGTGATGAGGGATGCCAGGG | 9229 |
| hs132668289 | TTTACGTGCTGAAAATTCTGGGG | 9230 |
| hs132668031 | TGACATGTGAATTAAATGGGAGG | 9231 |
| hs132668069 | CGGGGCTTTATGAAAGAGGGAGG | 9232 |
| hs132668070 | CCCGTTGCTGATGCATTGTGTGG | 9233 |
| hs132668425 | CTAGAATTATAATATACCCAGGG | 9234 |
| hs132668385 | TGGTAAAGAGGTACAGATGGGGG | 9235 |
| hs132667934 | CAGTGATGCGAGATACCCACAGG | 9236 |
| hs132668340 | AATGGTAAATCAAGAGAGCGTGG | 9237 |
| hs132668023 | GTCCATGCTCAGATCCACAACGG | 9238 |
| hs132668116 | TTAATCTAATCTGCTTCACAAGG | 9239 |
| hs132668049 | CCTGGCTCCAGTAAATTCAAGGG | 9240 |
| hs132668362 | AATCTGCAGTAACATTGCTGAGG | 9241 |
| hs132668458 | AAGATTGAAGACAAAAGAGGAGG | 9242 |
| hs132667892 | ATGTACGCTAGAAGTTGGAAGGG | 9243 |
| hs132668157 | CTACTGTCGTTATCAATAGGTGG | 9244 |
| hs132668503 | AATAGTAACTAACTGCGGATGGG | 9245 |
| hs132668363 | ATCTGCAGTAACATTGCTGAGGG | 9246 |
| hs132668477 | ACTCACAGATGGTTGTATGGTGG | 9247 |
| hs132668082 | TCCATTATGAGGCAGAAATGTGG | 9248 |
| hs132667931 | TACACGCATGGGTAAACAGAAGG | 9249 |
| hs132668447 | TTGGGACTGAAGGAACTGATGGG | 9250 |
| hs132668068 | CAACGGGGCTTTATGAAAGAGGG | 9251 |
| hs132668163 | GTTCTAAGACAAAAGTTGGGTGG | 9252 |
| hs132668260 | ATTGTGATGGTGAAAAAGGAAGG | 9253 |
| hs132667989 | TGTTAAACAAGAGTATGTATGGG | 9254 |
| hs132668133 | ATCCCAATTGAACAGACTGGTGG | 9255 |
| hs132667944 | TTACCTGACATGCACTGACAGGG | 9256 |
| hs132668234 | TGAAGTTCTCTACAGAATGAGGG | 9257 |
| hs132668421 | TGTTTAATACTGGACTGAGAGGG | 9258 |
| hs132668177 | AGAGTTGGCATGAGACATAGAGG | 9259 |
| hs132667923 | TGTGTTAATATCCTCTAGGTGGG | 9260 |
| hs132668108 | ATAATGGTATTCATTCAGTAGGG | 9261 |
| hs132668090 | TGAGAACCAAGCCATACCCTTGG | 9262 |
| hs132668030 | TCATATTGTTGTACGTGCCAAGG | 9263 |
| hs132668143 | CAAGCTACCAAGGAATGCCTGGG | 9264 |
| hs132667936 | TAAGCTCGCACATTTCCTGTGGG | 9265 |
| hs132668451 | CAGCATTAAAAGGAATCAAGTGG | 9266 |
| hs132667894 | TTCCAACTTCTAGCGTACATAGG | 9267 |
| hs132668046 | ATACTTAAGATTTACCTCCCAGG | 9268 |
| hs132668012 | GTAAGAAATTGGATAAAATGGGG | 9269 |
| hs132667994 | ATGGCATCACAAAAATGTATGGG | 9270 |
| hs132668246 | CAAAGAGGTCGTAACATACAGGG | 9271 |
| hs132668086 | ACTGAATAGTGTTTAGCCAAGGG | 9272 |
| hs132668440 | TAAAGTATAGAACTCAAGCATGG | 9273 |
| hs132668161 | TCTAAGACAAAAGTTGGGTGGGG | 9274 |
| hs132668014 | GGTTTCTAATCCTAATACAGTGG | 9275 |
| hs132668063 | ACCACACAATGCATCAGCAACGG | 9276 |
| hs132667917 | CTATAAAGTGCATCATAATGAGG | 9277 |
| hs132668222 | GTTAAAATAACAAAGCCACTTGG | 9278 |
| hs132668485 | TTTTCAATATAATTCATCCAAGG | 9279 |
| hs132668336 | CTGTTTCTAATAAAGAAAGAAGG | 9280 |
| hs132668145 | GAGGAAACCTCAAGCTACCAAGG | 9281 |
| hs132668277 | TTCCATTAACCACTCCTACAGGG | 9282 |
| hs132668134 | GGTATCCCAATTGAACAGACTGG | 9283 |
| hs132668242 | CAGAGTCGTCTGTTTAAACCCGG | 9284 |
| hs132667897 | AAGAGACAAGCAATATGAAGTGG | 9285 |
| hs132668278 | TCCATTAACCACTCCTACAGGGG | 9286 |
| hs132668379 | CTTCCTTATTGGACTAAATGGGG | 9287 |
| hs132668480 | TAACCACACAAGCATCACCTTGG | 9288 |
| hs132668383 | ACCTCTTTACCAAAGACAGCTGG | 9289 |
| hs132668005 | TTTGAGTGATTCCAGAGCTGAGG | 9290 |
| hs132668017 | CATTATTGCTAAAAGCTCAAGGG | 9291 |
| hs132667961 | TAAGCTTGTAACCCCATAGGTGG | 9292 |
| hs132667937 | TTAAGCTCGCACATTTCCTGTGG | 9293 |
| hs132668402 | GATGAGGGATGCCAGGGCGAAGG | 9294 |
| hs132668059 | GCGGAAACAATTATGTGCCATGG | 9295 |
| hs132668433 | GGACGAGAAGATGGTACAGAAGG | 9296 |
| hs132668249 | AGCTTGTATGAATCAAATGCAGG | 9297 |
| hs132668487 | GGGTCAGCTAATGATGAAATGGG | 9298 |
| hs132668432 | CGAGAAGATGGTACAGAAGGAGG | 9299 |
| hs132668312 | GGATCAGATTATTAAACATGAGG | 9300 |
| hs132668387 | TTTGGTAAAGAGGTACAGATGGG | 9301 |
| hs132668048 | CCTTGAATTTACTGGAGCCAGGG | 9302 |
| hs132668194 | ACCCACCATACAAAATTACAGGG | 9303 |
| hs132668111 | GCATCTGGATATCTCACTGTAGG | 9304 |
| hs132668403 | TGCCAGGGCGAAGGAAGAGAAGG | 9305 |
| hs132668253 | ACAAGTCTATAGAGACTGGTAGG | 9306 |
| hs132668155 | GTGCAAGACATTAACCTCAAAGG | 9307 |
| hs132668076 | AGAAATAAGGGATTTTCAGAGGG | 9308 |
| hs132668004 | TTGAGTGATTCCAGAGCTGAGGG | 9309 |
| hs132668450 | TCAAAGAAAAGTTATAGCAAGGG | 9310 |
| hs132668374 | ATTTTTAGAGTTCTAACTCAGGG | 9311 |
| hs132668367 | GTTTGAAGAAAACCAAAGACAGG | 9312 |
| hs132668087 | ATAGTGTTTAGCCAAGGGTATGG | 9313 |
| hs132668287 | ACGTAAATCAGATACTAGAAAGG | 9314 |
| hs132668131 | GATACCAGAAAATAAAGAGCAGG | 9315 |
| hs132668172 | CTTTACAATTTTAAACAACAGGG | 9316 |
| hs132668301 | AATTGGCTGATCCAATGGAATGG | 9317 |
| hs132668109 | GTTGTGTAGCACAAATTAGAAGG | 9318 |
| hs132668292 | CTTTCTCTATGAGTGTTCGTGGG | 9319 |
| hs132667927 | CCTAGTCACTTAGAAATAGGTGG | 9320 |
| hs132667891 | TATGTACGCTAGAAGTTGGAAGG | 9321 |
| hs132667930 | TTAAAAAAAAATACACGCATGGG | 9322 |
| hs132668164 | ATGGTTCTAAGACAAAAGTTGGG | 9323 |
| hs132668347 | CTAAGATCTAAATCATACATTGG | 9324 |
| hs132668015 | TTTTTTTTTCTCTAGAGAAGAGG | 9325 |
| hs132668055 | CTCTTAACGCCAATACTGATGGG | 9326 |
| hs132668067 | GCAACGGGGCTTTATGAAAGAGG | 9327 |
| hs132668346 | AGATCTAAATCATACATTGGAGG | 9328 |
| hs132668223 | AGTATCACACAGGTTACAAAGGG | 9329 |
| hs132668439 | ACTATTGGCTGGAATTGTCGTGG | 9330 |
| hs132668204 | TATGTACTAATGCAATTGAGTGG | 9331 |
| hs132668085 | TACTGAATAGTGTTTAGCCAAGG | 9332 |
| hs132667883 | CCAGGTATTGGGAGATCGATGGG | 9333 |
| hs132667960 | TTTTAAGCTTGTAACCCCATAGG | 9334 |
| hs132668268 | GAATTAGTTTGAGATACAAAGGG | 9335 |
| hs132668105 | AAAATTCCTTGAGTTCAGACAGG | 9336 |
| hs132668028 | CCCCGTTGTGGATCTGAGCATGG | 9337 |
| hs132668475 | ACAACCATCTGTGAGTTCTACGG | 9338 |
| hs132668267 | TTACATTTATATCTATAGATGGG | 9339 |
| hs132667915 | TTGTGATGCTTTCTATGCAAAGG | 9340 |
| hs132668060 | GCCTTAGTGGATATACGCCTGGG | 9341 |
| hs132667911 | ATTTTCTTCTACAATCCAAATGG | 9342 |
| hs132668455 | GACACTACAGAGCTATATAGAGG | 9343 |
| hs132668411 | ACTCAGAGAAGGATCTTAGCAGG | 9344 |
| hs132668146 | GGTATAAAACGATAAGGAAGAGG | 9345 |
| hs132667975 | AAATTCTGAGATGTACTAATGGG | 9346 |
| hs132667910 | TCTTCTACAATCCAAATGGTAGG | 9347 |
| hs132668457 | AAGAAGATTGAAGACAAAAGAGG | 9348 |
| hs132668002 | AAGACTATATCCCTCAGCTCTGG | 9349 |
| hs132668042 | GAGGAAAATCTGACAAGCCTGGG | 9350 |
| hs132668264 | TTAAAAAATAACTAATCATAGGG | 9351 |
| hs132668192 | GGAAATATAAGCAAGTATGCTGG | 9352 |
| hs132668269 | TGAATTAGTTTGAGATACAAAGG | 9353 |
| hs132668103 | TTTTAGCTGTCACTTCCAAAGGG | 9354 |
| hs132668390 | GCCAGCTGTCTTTGGTAAAGAGG | 9355 |
| hs132668321 | TTATATGGCAGAAATTCTCCTGG | 9356 |
| hs132668281 | TGTGTAAAGTGTGCCCCTGTAGG | 9357 |
| hs132668166 | ATTGAAAGATCCAAAAAAGATGG | 9358 |
| hs132668492 | GGGAAGCAGGGGTTGAAGGGAGG | 9359 |
| hs132668088 | GTTTAGCCAAGGGTATGGCTTGG | 9360 |
| hs132668179 | TTAGAATTTGTGATTTCTTGTGG | 9361 |
| hs132667952 | TAGATCTGCATCATCACGTCAGG | 9362 |
| hs132668247 | ACCTCTTTGCACATACCACCGGG | 9363 |
| hs132668427 | CTGGGTATATTATAATTCTAGGG | 9364 |
| hs132668142 | CCTGGGAATGTGATGAATGCTGG | 9365 |
| hs132668399 | TGAAGGAATTGTGGTGATGAGGG | 9366 |
| hs132668434 | AGAAGAAGAGGACGAGAAGATGG | 9367 |
| hs132668150 | GACTCTGAGCTCCTTGAGGTGGG | 9368 |
| hs132668149 | CTGCGAGGTATAAAACGATAAGG | 9369 |
| hs132668368 | TTTTCCAAAGAAGACTCTAAAGG | 9370 |
| hs132667957 | TCTTCTTCACTGGAGTTGATGGG | 9371 |
| hs132668202 | CTAATGCAATCGAGTGTGACAGG | 9372 |
| hs132668405 | AGGAAGAGAAGGCGTGAGATGGG | 9373 |
| hs132668391 | ATCCTTTTTGAGCCTCTGGATGG | 9374 |
| hs132668493 | GCTGGGAAGCAGGGGTTGAAGGG | 9375 |
| hs132667987 | TTTGCAATTCTTTAACGGGTTGG | 9376 |
| hs132668304 | CAATTTGTAGAAATACTTTGTGG | 9377 |
| hs132668258 | TATCAGAAGCAGACAGAAGTTGG | 9378 |
| hs132668446 | TTTGGGACTGAAGGAACTGATGG | 9379 |
| hs132668282 | GCCCCAGCTAAATCCTCAAATGG | 9380 |
| hs132667935 | GCATCACTGTTTGAGCGCTATGG | 9381 |
| hs132668300 | AATGGAATGGAATGAATTTGAGG | 9382 |
| hs132668137 | CCAGCATTCATCACATTCCCAGG | 9383 |
| hs132668141 | AGGCATTCCTTGGTAGCTTGAGG | 9384 |
| hs132668474 | AAACAAATACAGAAGAGTATAGG | 9385 |
| hs132667908 | TTTCAATTAAGAGTCTTTTGAGG | 9386 |
| hs132668497 | TTAAATAAGGCTGGGAAGCAGGG | 9387 |
| hs132668314 | TATTTTCAAGAAGGAAAACCTGG | 9388 |
| hs132668273 | TAAACCTTCTAAGTTAATATGGG | 9389 |
| hs132668348 | GCTTAAAGAAAACTTTCCTCAGG | 9390 |
| hs132668394 | TCAACAGAATTTTAGTGCAATGG | 9391 |
| hs132668305 | AATGTCATCTGATATAAAGTTGG | 9392 |
| hs132668024 | TCCATGCTCAGATCCACAACGGG | 9393 |
| hs132668352 | AATGAGCAAATGTTAAAGAATGG | 9394 |
| hs132668215 | AATAGAAGTTATTTATGAAGAGG | 9395 |
| hs132668317 | ATAAAAATAATTATAAAGAATGG | 9396 |
| hs132668489 | GCAGACATTTTTCTAAGGCATGG | 9397 |
| hs132668453 | ATCTAGACTAGTATCTAAAAGGG | 9398 |
| hs132668462 | GGAAGGCAGTAGCCATTAAAGGG | 9399 |
| hs132668315 | CAAGTATTCTATTTTCAAGAAGG | 9400 |
| hs132668007 | ATAGCCATTCAGGCAATGATAGG | 9401 |
| hs132668074 | TAGCAGAGAAGTGTATTATAGGG | 9402 |
| hs132668032 | ATATGACATGTGAATTAAATGGG | 9403 |
| hs132667890 | CTCCTATGTACGCTAGAAGTTGG | 9404 |
| hs132668044 | TGGGAGGTAAATCTTAAGTATGG | 9405 |
| hs132668437 | ATCTCTGTTTCAAAGTCAAATGG | 9406 |
| hs132667949 | GCATCATCACGTCAGGTAGATGG | 9407 |
| hs132667966 | AATTATACATTTCCACCTATGGG | 9408 |
| hs132667995 | TATGGCATCACAAAAATGTATGG | 9409 |
| hs132667980 | GATATAAAGAACACTTTAAGTGG | 9410 |
| hs132668372 | TACATGCAGATATATCCTATTGG | 9411 |
| hs132668173 | CAACAGGGCCATATTCCAATAGG | 9412 |
| hs132668110 | ATCCAGATGCCGAATCCCTTTGG | 9413 |
| hs132668471 | CAAAGACATCTACTAAATATCGG | 9414 |
| hs132668276 | ATTCCATTAACCACTCCTACAGG | 9415 |
| hs132668375 | ACTCTAAAAATACTTCCAATAGG | 9416 |
| hs132668328 | GAATAACATACCTAAAAATAGGG | 9417 |
| hs132668406 | CGCCTTCTCTTCCTTCGCCCTGG | 9418 |
| hs132668190 | TCCCCTGTAATTTTGTATGGTGG | 9419 |
| hs132667904 | GTAAAAGGTGTATTTTCCTTAGG | 9420 |
| hs132668369 | TTTAGAGTCTTCTTTGGAAAAGG | 9421 |
| hs132668156 | CTACTACTGTCGTTATCAATAGG | 9422 |
| hs132668279 | GCCCCTGTAGGAGTGGTTAATGG | 9423 |
| hs132668358 | GCGTAGAGAGTCTGTTTCATAGG | 9424 |
| hs132667981 | TTATGATTCAGCTAATATCTAGG | 9425 |
| hs132668165 | GATGGTTCTAAGACAAAAGTTGG | 9426 |
| hs132668407 | GATGGGCACTCAACACTAATAGG | 9427 |
| hs132668270 | GTTTTCTGACTTAAAACAATAGG | 9428 |
| hs132668393 | TGCCATCCAGAGGCTCAAAAAGG | 9429 |
| hs132667909 | AATGAGAATTGCCTACCATTTGG | 9430 |
| hs132668378 | TCTTCCTTATTGGACTAAATGGG | 9431 |
| hs132668189 | ATTTCCCCTGTAATTTTGTATGG | 9432 |
| hs132667939 | ACTGCTTCGTAATAATTAGATGG | 9433 |
| hs132668235 | ATGCTTCTACTAGATAAGATTGG | 9434 |
| hs132668241 | ATATCCAACCGTCTGTTCATAGG | 9435 |
| hs132668020 | TGTGAGACTTTGTAATTCATTGG | 9436 |
| hs132668019 | AATTACAAAGTCTCACAAAATGG | 9437 |
| hs132667943 | TAAAATGGCACGTAACAAAATGG | 9438 |
| hs132668170 | ACACAGTGAAACATTTTAGAAGG | 9439 |
| hs132667941 | TCAGTGCATGTCAGGTAAAATGG | 9440 |
| hs132668239 | AAGATTTTTTTGACTTAAAAGGG | 9441 |
| hs132667928 | TCTCCTAGTCACTTAGAAATAGG | 9442 |
| hs132668364 | GAGAGAAAGAATCTAATTATCGG | 9443 |
| hs132667979 | TGTTCTTTATATCCAAATAATGG | 9444 |
| hs132668084 | GCCACATTTCTGCCTCATAATGG | 9445 |
| hs132668016 | CTGAGTTTCTATTTACGTATAGG | 9446 |
| hs132668091 | TGAAGCACCATACTATAAAATGG | 9447 |
| hs132667899 | TATTATACCACTATATAAAATGG | 9448 |
| hs132668237 | GTGGAGTGCAAGAAAATAAAAGG | 9449 |
| hs132667903 | TTGCAGTGAGAACAAGTAAAAGG | 9450 |
| hs132667999 | AATATTGCTAAGCAATAAATAGG | 9451 |
| hs132668356 | TATATAGTCTTCAGTTTTAGAGG | 9452 |
| hs132668444 | ACTCAAGCATGGATTCTTTTGGG | 9453 |
| hs132668322 | GAGAATTTCTGCCATATAAAAGG | 9454 |
| hs132668125 | TCACTGCTGTTATTCTTTGCAGG | 9455 |
| hs132668361 | AGTCTTGTCCTTAACTTTAGAGG | 9456 |
| hs132667906 | AGTTAGACTTTTATTCTTTAGGG | 9457 |
| hs132668009 | GTTAGACAAGTGTAAGAAATTGG | 9458 |
| hs132668230 | TCACTGTTGGTCAAAATAATTGG | 9459 |
| hs132667870 | CTAGC | 9460 |
| | TGTAAAAGACACGGGG | |
| | GG | |
| hs132667871 | ACTAG | 9461 |
| | CTGTAAAAGACACGGG | |
| | GG | |
| hs132667872 | AACTA | 9462 |
| | GCTGTAAAAGACACGG | |
| | GG | |
| hs132667874 | GAAAC | 9463 |
| | TAGCTGTAAAAGACAC | |
| | GG | |
| hs132667867 | TGCAG | 9464 |
| | TGCCTTTTTAGTGCAT | |
| | GG | |
| hs132667744 | GTAAATGTATTTCTGCTGTGGGG | 9465 |
| hs132667669 | AGAGCTCTAGAAAATTCAGGTGG | 9466 |
| hs132667700 | GCGTATTCCTGATGAGAGTGAGG | 9467 |
| hs132667816 | GGAAGATGTTTGATGGTACGAGG | 9468 |
| hs132667653 | CCCTCAGTTAATAATCCTGGGGG | 9469 |
| hs132667789 | AGAGCATTTTTATGGCCCTGAGG | 9470 |
| hs132667773 | TAGTGAAAACTGGTGACAGATGG | 9471 |
| hs132667734 | GCTGGGGAAGGACAGCTCTGGGG | 9472 |
| hs132667541 | TGAACCGAATGTTAAAACTGTGG | 9473 |
| hs132667804 | TCGAGATCAAGAAAAAACACAGG | 9474 |
| hs132667633 | TCATTTGTTTAGGTACCATGAGG | 9475 |
| hs132667762 | CAAGCAGGACACAATCCCATGGG | 9476 |
| hs132667680 | GGAATGTAGACCTACCTCCAAGG | 9477 |
| hs132667801 | ACACTAAGACATCCAGACTCAGG | 9478 |
| hs132667610 | GTGGGGGTTTAGCAATAGGTAGG | 9479 |
| hs132667649 | CCAGGATTATTAACTGAGGGAGG | 9480 |
| hs132667573 | TGTATTTGCTGCCAAATATGGGG | 9481 |
| hs132667770 | TAGTATCACTGATAACCCATGGG | 9482 |
| hs132667608 | AAATAAAACTCACGGTGTGGGGG | 9483 |
| hs132667844 | TCTCATTCTCAAAACACTCGAGG | 9484 |
| hs132667631 | AAAACCTATGAGTAACCTCATGG | 9485 |
| hs132667683 | GACCTACCTCCAAGGTAGGGAGG | 9486 |
| hs132667551 | AAGTTCCTGTGAGCTGTGGAGGG | 9487 |
| hs132667748 | AGAATCACGTAAATTATCGTGGG | 9488 |
| hs132667600 | ACTGAACTATAACATACTCCAGG | 9489 |
| hs132667790 | AGAAATCTTGGCTAGCCTCAGGG | 9490 |
| hs132667636 | TGGCTTAATAATAAAACAGGAGG | 9491 |
| hs132667555 | TGTAACCCTCCACAGCTCACAGG | 9492 |
| hs132667566 | CTGACTTAATCTGTAAACAAAGG | 9493 |
| hs132667654 | TCCCTCAGTTAATAATCCTGGGG | 9494 |
| hs132667839 | TTAAACTCATAGTATCAGACCGG | 9495 |
| hs132667704 | GTAACTACACATAATATAGGTGG | 9496 |
| hs132667648 | CCCCCAGGATTATTAACTGAGGG | 9497 |
| hs132667516 | AAAGATCACAGAATAAGCCTGGG | 9498 |
| hs132667675 | AGAGAATGTGTACAAGGCAAAGG | 9499 |
| hs132667861 | TGAAAGCTAGAAAGTACATACGG | 9500 |
| hs132667761 | GCAAGCAGGACACAATCCCATGG | 9501 |
| hs132667565 | GGGAGTAATGTTCAGTACCCAGG | 9502 |
| hs132667682 | GTAGACCTACCTCCAAGGTAGGG | 9503 |
| hs132667778 | TTCACTGCCTGAGTCAGCAAAGG | 9504 |
| hs132667529 | GAATTCCGTCAGGAATTAAGGGG | 9505 |
| hs132667771 | ATAGTATCACTGATAACCCATGG | 9506 |
| hs132667593 | TGAACTGCAGAATGCTCTAGTGG | 9507 |
| hs132667629 | ATTTGGAAACATGTCAGAAGAGG | 9508 |
| hs132667588 | CTAAGTAGCCCATTTTGGCATGG | 9509 |
| hs132667818 | TTTTTGTAATTCTTTTTGGTAGG | 9510 |
| hs132667568 | TTTGTTTACAGATTAAGTCAGGG | 9511 |
| hs132667538 | ATAATCACAGCATTTCAGAATGG | 9512 |
| hs132667514 | GTTTTAACAGGTTTTAAAAGCGG | 9513 |
| hs132667840 | TCATTTCTAGTAAAAGAGGCCGG | 9514 |
| hs132667724 | AGATATTCTCAAATGGCTGTGGG | 9515 |
| hs132667666 | ACATATCAATAATTAACCATAGG | 9516 |
| hs132667578 | TGTCATGACAATTTGCTCTGTGG | 9517 |
| hs132667622 | TCTGAGTAAGTAAGTCAAAGAGG | 9518 |
| hs132667852 | GCTGCTTAGAACTTTATGAAAGG | 9519 |
| hs132667646 | CCCAGTTCCAGAACTCCCCCAGG | 9520 |
| hs132667835 | TGGTACGTATTTAAAATGAGTGG | 9521 |
| hs132667589 | TTAGATTGTCCTTTTCAGGAAGG | 9522 |
| hs132667854 | AAGTGGGAAAAAACTATAGGTGG | 9523 |
| hs132667713 | AATTATTTGAGGGCCATCCCAGG | 9524 |
| hs132667527 | GATTTCCCCTTAATTCCTGACGG | 9525 |
| hs132667694 | TGTATCCTTACAAAAGGCCCAGG | 9526 |
| hs132667768 | TGTCCTGCTTGCCATTGAGGTGG | 9527 |
| hs132667536 | TGAGAAAATGAGAATGGCTAAGG | 9528 |
| hs132667546 | AGTAAAAGAACTAAGTCGCTGGG | 9529 |
| hs132667708 | ATCACCAGTTGTACTTTGGGAGG | 9530 |
| hs132667728 | TAAGGGCTACAGAAGTACTTAGG | 9531 |
| hs132667687 | GTACAGTCAATAAATCAGACTGG | 9532 |
| hs132667732 | AGGACAGCTCTGGGGAAGCAGGG | 9533 |
| hs132667723 | TCAAATGGCTGTGGGATCAAAGG | 9534 |
| hs132667572 | CCAAATATGGGGTTGTTAAGTGG | 9535 |
| hs132667559 | ATTCCTTTCAAATCAACCAAAGG | 9536 |
| hs132667719 | CTACCTTTACCTCAACTCCTGGG | 9537 |
| hs132667643 | ATGAGTATCAAAAATGATGATGG | 9538 |
| hs132667794 | AGTTGCAGTTATTTTTTCCAAGG | 9539 |
| hs132667518 | TGGTGTATGGAATGCAACCCAGG | 9540 |
| hs132667725 | AAGATATTCTCAAATGGCTGTGG | 9541 |
| hs132667759 | ATCCCCACCTCCCACCTCAATGG | 9542 |
| hs132667614 | TATGGCTCTCAGCTTGGGACAGG | 9543 |
| hs132667612 | TTGCATTAGTACTAGAACAAAGG | 9544 |
| hs132667533 | GTTTTCGTTTGAATTCCGTCAGG | 9545 |
| hs132667624 | GCCATATGTGTAAGGCTGAATGG | 9546 |
| hs132667743 | ATTTCTGCTGTGGGGACTGTTGG | 9547 |
| hs132667644 | ACTCATATCTAAAATTTACTAGG | 9548 |
| hs132667780 | CAAATTCTAGACCAGCTGTCAGG | 9549 |
| hs132667856 | CTACTTTTCTGAATTAAAGTGGG | 9550 |
| hs132667823 | AGTTTTGGCCTGCAGCTGATGGG | 9551 |
| hs132667717 | GTAAAGGTAGCAAGTCAAACAGG | 9552 |
| hs132667672 | TGAATTTTCTAGAGCTCTATGGG | 9553 |
| hs132667710 | TCAACCTCCCAAAGTACAACTGG | 9554 |
| hs132667522 | TCAAAAGTTCAGAAAAATAGAGG | 9555 |
| hs132667859 | ATAGTGATAATATACAGTACTGG | 9556 |
| hs132667795 | CCAAGGAATCAACTGATATGAGG | 9557 |
| hs132667689 | TACCTTCCTCCCTACCTTGGAGG | 9558 |
| hs132667558 | TTCCTTTCAAATCAACCAAAGGG | 9559 |
| hs132667596 | GTAAAAGACAAACTAATTGAAGG | 9560 |
| hs132667712 | TGTCTGAATTAATTATTTGAGGG | 9561 |
| hs132667632 | GGTACCATGAGGTTACTCATAGG | 9562 |
| hs132667830 | TTAAATTAAAAGACCAATGAGGG | 9563 |
| hs132667645 | ATGATGGCAGATTAGTTGTTTGG | 9564 |
| hs132667635 | AAGCCACATGAAGAAACATTTGG | 9565 |
| hs132667820 | ATTCTTTTGCAAACTCTCAATGG | 9566 |
| hs132667539 | GTTTCCACAGTTTTAACATTCGG | 9567 |
| hs132667772 | CCTAGACATGTAGTGAAAACTGG | 9568 |
| hs132667832 | CCATAAATGCTAACCCTCATTGG | 9569 |
| hs132667554 | TCCACAGCTCACAGGAACTTTGG | 9570 |
| hs132667553 | GGAGGGTTACAAACTCCCTTTGG | 9571 |
| hs132667806 | TTACACATGTAAAGTTCATTAGG | 9572 |
| hs132667809 | CTAAGAACAACTTTCTTGATTGG | 9573 |
| hs132667547 | ACTTAGTTCTTTTACTTAATGGG | 9574 |
| hs132667601 | GCTTTGTTTCCCCTTAAAAATGG | 9575 |
| hs132667543 | TGGATGCTACAAAAATTTTATGG | 9576 |
| hs132667544 | ACCAGATGTTAATTGATTAATGG | 9577 |
| hs132667845 | GAGATTTCTTTGGCAATAAATGG | 9578 |
| hs132667105 | AGACAGGAGAGGGACTACGAAGG | 9579 |
| hs132667274 | GTAGACCTCTTTGTACAAGGGGG | 9580 |
| hs132667013 | ATAATGATTACCATCCCAAGAGG | 9581 |
| hs132667332 | AATACCTGGACACAAGCTGAGGG | 9582 |
| hs132667188 | ATAAGTTTTAATGGGCATGGAGG | 9583 |
| hs132667380 | TAGACAAGAAAGCATTTGAGGGG | 9584 |
| hs132667017 | GTTTCCAAGGAAGTCACAAGAGG | 9585 |
| hs132667103 | TTAGGAAATATAGACAGGAGAGG | 9586 |
| hs132667484 | CTTCCAGCCCAATAGTAACGGGG | 9587 |
| hs132667130 | AAACCATCATCTATGACATGAGG | 9588 |
| hs132667001 | AGGCCATCACCTCAATACATGGG | 9589 |
| hs132667397 | AAATTATGAGACCACCACCTAGG | 9590 |
| hs132667357 | GGAAAAGAGAAATTAAAAGGAGG | 9591 |
| hs132667036 | ATCTCCCTGCCTCATACACGAGG | 9592 |
| hs132667046 | GCTGAAATAAGAAGATCCTGAGG | 9593 |
| hs132666968 | GTCAAACTGTAGGAAATAAGGGG | 9594 |
| hs132667205 | AAGCTGAGTTACTAATCACAAGG | 9595 |
| hs132667438 | GGCAGATCACATAAAACAGGAGG | 9596 |
| hs132667203 | AAAAGATCAGATGGTCACAAAGG | 9597 |
| hs132666998 | TTTGTGAGCCAAGACATGAGAGG | 9598 |
| hs132667407 | TCACATTTCAGGGAGCATTGTGG | 9599 |
| hs132667383 | ATACAATGAAGATAAAATGGAGG | 9600 |
| hs132667111 | TATTCAGCAGCTGAACCACAAGG | 9601 |
| hs132667342 | TTCTTTATATGAGTACAACTGGG | 9602 |
| hs132666994 | CTGCCAAAAGTATGAACAGGAGG | 9603 |
| hs132666955 | TCAGTAAAGACTGCTTGGAGTGG | 9604 |
| hs132667362 | CTGTTTTTGAGCTCACAGGAAGG | 9605 |
| hs132667333 | TAATACCTGGACACAAGCTGAGG | 9606 |
| hs132666972 | GTTTGACCAATATATTGAAGGGG | 9607 |
| hs132667096 | AGTTCTACCCTATATTTACAGGG | 9608 |
| hs132667037 | GGACTGATTCCTTTCATGTGAGG | 9609 |
| hs132666952 | TGTGAGAAGTATTGAAACAGAGG | 9610 |
| hs132667435 | ATGTATTGCAAAGAAGCCATGGG | 9611 |
| hs132667038 | CAGTCCTCGTGTATGAGGCAGGG | 9612 |
| hs132667033 | TTTGCCAAGAGGTAAGGGAAGGG | 9613 |
| hs132667326 | TAATAACTCTTGCTATCTGGAGG | 9614 |
| hs132667052 | ACATATCTTTACCTATGTGAGGG | 9615 |
| hs132667011 | AACGTTACAATCGTCATGAGAGG | 9616 |
| hs132667335 | TGAAAAAATAATCAGCTAGGAGG | 9617 |
| hs132667178 | AATACTGAAATTGTAAACAATGG | 9618 |
| hs132667134 | ACAATGCTAGACATAAACATTGG | 9619 |
| hs132667222 | CAGTGTTAGATGGTATGTAGAGG | 9620 |
| hs132667493 | TTTTTATTTAACTAAACACAGGG | 9621 |
| hs132667088 | CAACATGATATACTGTGAAGCGG | 9622 |
| hs132667149 | AGCATCCTGATATTATTCCAGGG | 9623 |
| hs132667401 | AAAAAGTAGTCCCATTCCAAGGG | 9624 |
| hs132667426 | TTTATGGCAGCAATAATCTGTGG | 9625 |
| hs132667385 | ATACATGCTGTAATGCATGTTGG | 9626 |
| hs132667489 | GTCTTGTTGTTGGAGAATGGTGG | 9627 |
| hs132667320 | TATGTTTATAAAAAATTACAGGG | 9628 |
| hs132667456 | TCTATATTGAGACAAGTACATGG | 9629 |
| hs132667424 | AATGGCAATCTTTCTTAACACGG | 9630 |
| hs132667192 | AAAAACCAACTCCACAGGCATGG | 9631 |
| hs132667488 | GTTGTTGGAGAATGGTGGTGAGG | 9632 |
| hs132667481 | GGAACTGAGTGACTTAGAGCAGG | 9633 |
| hs132667392 | CATCACGTCAATTGCCTAGGTGG | 9634 |
| hs132667016 | ATATAAGTTGGATTGCCAGAAGG | 9635 |
| hs132667416 | GAGCTGTAACAGTTAACTGTAGG | 9636 |
| hs132667271 | TATATCCCCCTTGTACAAAGAGG | 9637 |
| hs132667348 | AATGGTTAAAACCTATTGTGAGG | 9638 |
| hs132667471 | TTAGTTATACTAACACATACGGG | 9639 |
| hs132667112 | AGAATTTTATAGAAACCTTGTGG | 9640 |
| hs132667120 | GCATACTGGTATTTTTCACGTGG | 9641 |
| hs132667236 | TTTTTTAGCTGCCTTTCCAGTGG | 9642 |
| hs132667476 | GAGCAGGCGAACAAAATGCTGGG | 9643 |
| hs132667322 | TTTTTAAAGAAGTCAATCCAGGG | 9644 |
| hs132667324 | AGGCTTACAATGAGAAAAGAGGG | 9645 |
| hs132667197 | GCCTTGTGCTACCATGCCTGTGG | 9646 |
| hs132667073 | ACTTTCTTTGCATACAGTGGAGG | 9647 |
| hs132667454 | ACATCTGATAAGTAGCACTTAGG | 9648 |
| hs132666963 | TTGGTTCACTGAAATAGCAAGGG | 9649 |
| hs132667237 | GCCTTTCCAGTGGCAAAAGTAGG | 9650 |
| hs132667104 | TAGGAAATATAGACAGGAGAGGG | 9651 |
| hs132667391 | AGACATCACGTCAATTGCCTAGG | 9652 |
| hs132667459 | AAATTCATAGCTCAATGCACAGG | 9653 |
| hs132667132 | TTATGGTATATGAATGTGGATGG | 9654 |
| hs132667235 | AACATCCTATCCCTATTCAAAGG | 9655 |
| hs132667446 | ACTGTGATTGAAAATCATATAGG | 9656 |
| hs132667483 | GCTGGAAGAAATTCACTCTAGGG | 9657 |
| hs132667277 | TTGTAGACCTCTTTGTACAAGGG | 9658 |
| hs132667220 | GTATGTAGAGGAAGGCCAGATGG | 9659 |
| hs132666997 | GATGGATGAACTGTTGCCCCCGG | 9660 |
| hs132667202 | TCCTTGGGCAAAAGATCAGATGG | 9661 |
| hs132667028 | GCTCCTTTGCTTGTAACCAGAGG | 9662 |
| hs132667226 | ACACATTCTATAATTAGGGATGG | 9663 |
| hs132667313 | AATCACATGTGCTGTTGAAAGGG | 9664 |
| hs132667299 | GAACTGCCATGTACAAGAAGAGG | 9665 |
| hs132667451 | AAGTTTGTAGTATTGCCTCCAGG | 9666 |
| hs132667487 | CCATTCTCCAACAACAAGACAGG | 9667 |
| hs132667109 | TGTCCAAAGCCAAGAAACATTGG | 9668 |
| hs132666988 | CCTGTTCATACTTTTGGCAGCGG | 9669 |
| hs132667444 | TTATTATGTAATGATTGTGTGGG | 9670 |
| hs132667247 | TTACAGACAATGCATTTACAAGG | 9671 |
| hs132666974 | AGTTTGACCAATATATTGAAGGG | 9672 |
| hs132667146 | ATGTAATCTTATGAGTAAGAAGG | 9673 |
| hs132667334 | GATTTGACTTGAATAATACCTGG | 9674 |
| hs132667152 | CTGCAAGAAGAGGCACACCCTGG | 9675 |
| hs132667431 | TGGAAGCAATGGGTACATATGGG | 9676 |
| hs132667083 | TGGATACATGTGCCCATGGAGGG | 9677 |
| hs132667139 | TCACTCAATGCATTAGAATGAGG | 9678 |
| hs132667208 | TGTATGCTACATTCAGCTATGGG | 9679 |
| hs132667014 | GGCAATCCAACTTATATGCAAGG | 9680 |
| hs132667093 | TTCTTATTTCTCCATCCCGAAGG | 9681 |
| hs132667291 | TGCCGAAGCAAGCACCTGATGGG | 9682 |
| hs132667317 | ATATATATCGATCAGGGCCAAGG | 9683 |
| hs132667376 | TGTAGTTCAGTGTGTCTGGAGGG | 9684 |
| hs132666962 | TTTGGTTCACTGAAATAGCAAGG | 9685 |
| hs132667330 | AATACCCTCAGCTTGTGTCCAGG | 9686 |
| hs132667144 | CTATCTTTTCTAAACGCTCAGGG | 9687 |
| hs132667021 | CTGCCTCTGGTTACAAGCAAAGG | 9688 |
| hs132667062 | GAGAAGATAAAACCTGTGCTTGG | 9689 |
| hs132667338 | GACCTTTTGAAGCAGTTGCATGG | 9690 |
| hs132667053 | TTACCTATGTGAGGGACTGAAGG | 9691 |
| hs132667145 | TGAGCGTTTAGAAAAGATAGTGG | 9692 |
| hs132667381 | ATAGACAAGAAAGCATTTGAGGG | 9693 |
| hs132667465 | TTTTGTCTTATGTAGTTGGATGG | 9694 |
| hs132667437 | ATGGCTTCTTTGCAATACATTGG | 9695 |
| hs132667234 | AGTCTCCTTTGAATAGGGATAGG | 9696 |
| hs132667170 | TGAGCATAGCTGATAAGAGTTGG | 9697 |
| hs132667227 | GGATGGATATTATCAAGTATAGG | 9698 |
| hs132667077 | TATAACTGCATGAATTAGCTTGG | 9699 |
| hs132667228 | ATATTATCAAGTATAGGCTTAGG | 9700 |
| hs132667395 | TGGTGGTCTCATAATTTCTAGGG | 9701 |
| hs132667060 | TTTCTACATTTGCCAAGCACAGG | 9702 |
| hs132667447 | TGATTGAAAATCATATAGGCTGG | 9703 |
| hs132667486 | TTCTTCCAGCCCAATAGTAACGG | 9704 |
| hs132666983 | TGAGTTCCCATATTCATCACTGG | 9705 |
| hs132667455 | ATGTACTTGTCTCAATATAGAGG | 9706 |
| hs132667300 | AATTTGCCTCTTCTTGTACATGG | 9707 |
| hs132667042 | GGAATCAGTCCTCGTGTATGAGG | 9708 |
| hs132667287 | TTGGGTATAATAAAAAGCACTGG | 9709 |
| hs132667155 | TGTGTTATTTTGATAGGCCTGGG | 9710 |
| hs132667092 | CTACTTCATTTCCTTCGGGATGG | 9711 |
| hs132667477 | AGAGCAGGCGAACAAAATGCTGG | 9712 |
| hs132667184 | TCTATGTATATGTAACCCTTTGG | 9713 |
| hs132667439 | TGTTTTATGTGATCTGCCCATGG | 9714 |
| hs132667000 | GAGGCCATCACCTCAATACATGG | 9715 |
| hs132667427 | GCAATAATCTGTGGAAGCAATGG | 9716 |
| hs132667159 | ATGTGCATTTTTTAAACAAGTGG | 9717 |
| hs132667098 | TTATTCTGATTGTATGAAGTAGG | 9718 |
| hs132667031 | CTGAATTTGCCAAGAGGTAAGGG | 9719 |
| hs132667189 | AACTTATTGATCACTCCAAAGGG | 9720 |
| hs132667412 | ATGCTCCCTGAAATGTGAAAAGG | 9721 |
| hs132667289 | GGAGTTTTAAATCTCCCATCAGG | 9722 |
| hs132667480 | GTTCCCCGTTACTATTGGGCTGG | 9723 |
| hs132667119 | ATCGTCCCTATTTTGCCGTAAGG | 9724 |
| hs132667418 | CCTAAACATGTCCCTTAAAAGGG | 9725 |
| hs132667301 | ATATGTATTTTATCTTAAAGAGG | 9726 |
| hs132667464 | CAACTACATAAGACAAAAAGAGG | 9727 |
| hs132667201 | TGAACTTTGGGAGAATCCTTGGG | 9728 |
| hs132667086 | CATTTCCCTTTACCCTCCATGGG | 9729 |
| hs132667337 | TGCCATGCAACTGCTTCAAAAGG | 9730 |
| hs132666989 | CTGTTCATACTTTTGGCAGCGGG | 9731 |
| hs132667350 | ACATTTTCTATAATGATTGTGGG | 9732 |
| hs132667213 | ATATTATATGATAATTAAGAAGG | 9733 |
| hs132667194 | GTGGAGTTGGTTTTTAAGCATGG | 9734 |
| hs132667039 | TCAGTCCTCGTGTATGAGGCAGG | 9735 |
| hs132667294 | CAGGATATAGATGATATAGAAGG | 9736 |
| hs132667151 | GCACACCCTGGAATAATATCAGG | 9737 |
| hs132667399 | ACTGATTTGATTATTCCAATTGG | 9738 |
| hs132667018 | AACAGACCTTGCATATAAGTTGG | 9739 |
| hs132667157 | GAGCATAGTTTGTTTTTCCCAGG | 9740 |
| hs132667415 | TCTAGTTCATTTGCATTGCATGG | 9741 |
| hs132667297 | TTTTGGGCATCCTTGAATGTAGG | 9742 |
| hs132667085 | ATGTGCCCATGGAGGGTAAAGGG | 9743 |
| hs132667463 | ACATATTCTGCCACATAGCCTGG | 9744 |
| hs132667206 | CTTTATCTTATGAAGTATAGAGG | 9745 |
| hs132666957 | GAGAAACTGTTCTTGCTTGTTGG | 9746 |
| hs132667494 | ACACAGGGCAAAAACTAATCTGG | 9747 |
| hs132667095 | GCATTCACCCTGTAAATATAGGG | 9748 |
| hs132667250 | TCTCAAATACCTATTATAGTTGG | 9749 |
| hs132667136 | AATATCTTATAGAAGAACTTTGG | 9750 |
| hs132667440 | ATAGTACTAAAACTACTATTAGG | 9751 |
| hs132667485 | TCTTCCAGCCCAATAGTAACGGG | 9752 |
| hs132667254 | AGTTACATCTCAAATAACTTTGG | 9753 |
| hs132667117 | AGGAACCTTACGGCAAAATAGGG | 9754 |
| hs132666977 | AATGTTCTAGGCAATGGAATTGG | 9755 |
| hs132667078 | GCACAAATGTTCTATGAGATTGG | 9756 |
| hs132667318 | GCAGTTTTTTCTAATTGCCTTGG | 9757 |
| hs132667496 | ATCTGGTTGCTTCTTTTGTAGGG | 9758 |
| hs132667421 | TTGACGTGCATCCCTTTTAAGGG | 9759 |
| hs132667225 | TTCATTACTTCAGTGTTAGATGG | 9760 |
| hs132667233 | AATTAAGTCTCCTTTGAATAGGG | 9761 |
| hs132667186 | AGTGATCAATAAGTTTTAATGGG | 9762 |
| hs132667365 | GGATTGATCCAAATCTTTGCTGG | 9763 |
| hs132667370 | ATTTATTTCTAGCCCACAATAGG | 9764 |
| hs132667331 | GGGTATTTAAGTTGATTAGAAGG | 9765 |
| hs132667498 | ATAGTCTGTAGCATGATAATTGG | 9766 |
| hs132667268 | GTTTAATTAAAAATACTCTAAGG | 9767 |
| hs132667224 | TGAAACACATTCTATAATTAGGG | 9768 |
| hs132667091 | AATACTACTTCATTTCCTTCGGG | 9769 |
| hs132666992 | ATACTTTTGGCAGCGGGCCGGGG | 9770 |
| hs132666967 | GGTCAAACTGTAGGAAATAAGGG | 9771 |
| hs132667212 | ATTATTGTCAAATACTTAGAAGG | 9772 |
| hs132667429 | AGATTATTGCTGCCATAAAATGG | 9773 |
| hs132666960 | TGTTTTATCTGACTTAAGTTTGG | 9774 |
| hs132667099 | TCATGTACTAAATTTCCTTATGG | 9775 |
| hs132667387 | GTATCTCTTCACTTGCTCATAGG | 9776 |
| hs132667371 | GTGGGCTAGAAATAAATAAAAGG | 9777 |
| hs132666964 | TTAGCGCCCCTTCAATATATTGG | 9778 |
| hs132667319 | GTATGTTTATAAAAAATTACAGG | 9779 |
| hs132667071 | AAGAAAATGCAGTTTTTAGTAGG | 9780 |
| hs132667230 | TTTTTTTGTCAGGAGATAATAGG | 9781 |
| hs132667110 | TAGAATTCTTTGATTGACTATGG | 9782 |
| hs132667479 | CTCAGTTCCCCGTTACTATTGGG | 9783 |
| hs132667059 | ATCCTTTTAGCCTACCAAAATGG | 9784 |
| hs132667267 | GTATTTTTAATTAAACTTACTGG | 9785 |
| hs132666987 | TTACCTCCTGTTCATACTTTTGG | 9786 |
| hs132667045 | TCTTATTTCAGCTTCCAAATTGG | 9787 |
| hs132667165 | ATAATATGTAAATAATTAAATGG | 9788 |
| hs132667265 | ACTAGTCGTGGAGAGCTTTCAGG | 9789 |
| hs132667253 | TTGCTATCTCCAACTATAATAGG | 9790 |
| hs132666675 | AGTCTCACAAAACTGCAGAGCGG | 9791 |
| hs132666123 | GCTTTGTGGAGTCAACACCATGG | 9792 |
| hs132666149 | TAACCTATTCATAAGCACAGAGG | 9793 |
| hs132666112 | ATGGTTTAGACACAGAACTGGGG | 9794 |
| hs132666823 | CTTCAGATGATACAAAAACGGGG | 9795 |
| hs132666413 | CAAGGAGTGTGAAAACACGTGGG | 9796 |
| hs132666766 | CTGAGCCATATACCTCACTGTGG | 9797 |
| hs132666773 | ACAGAACTAGAGATGTCAGGTGG | 9798 |
| hs132666374 | AACAATGTGACAGATGACAGAGG | 9799 |
| hs132666082 | GTGAATTTGAAAGTGACTGGTGG | 9800 |
| hs132666415 | GCAAAAACTGCATGACCCAGAGG | 9801 |
| hs132666761 | GGGCAGTTCTCTCCACAGTGAGG | 9802 |
| hs132666915 | CTATAGGTGATAAACTACTGTGG | 9803 |
| hs132666190 | ACTCAGGGAGAGTACATGGGAGG | 9804 |
| hs132666329 | CCATGTGGAAACAAGCAGAGAGG | 9805 |
| hs132666571 | GATGACTTGGACCCTGTGGGAGG | 9806 |
| hs132666871 | TCCAAGCACTCTACTCACGATGG | 9807 |
| hs132666787 | AAGATGGAGGAAGATTGTGGTGG | 9808 |
| hs132666876 | TAGCCAAGCTGACACAACCAGGG | 9809 |
| hs132666259 | TTCAAAATATCAAAAGCCTGGGG | 9810 |
| hs132666201 | GGTCTGAGTAGGATCCGGGAAGG | 9811 |
| hs132666689 | AAAACAGACCAGGTGAACTGAGG | 9812 |
| hs132666451 | AAGCATGACTCTCCAAACTGTGG | 9813 |
| hs132666923 | ACTGACATTACAGGTACCCGAGG | 9814 |
| hs132666444 | GTGTACCTCAAAATGGCTGAAGG | 9815 |
| hs132666786 | AGATGGAGGAAGATTGTGGTGGG | 9816 |
| hs132666092 | CATGCCAGCCTAGTACCTTGAGG | 9817 |
| hs132666499 | TATTACAACATAGCAAGGCGAGG | 9818 |
| hs132666352 | TATCTTCCCCCCTTCCACAGAGG | 9819 |
| hs132666277 | AGAACACAGTGCCCATCAGGAGG | 9820 |
| hs132666147 | ATTTTACTAGGGCAGTGGTGAGG | 9821 |
| hs132666510 | CTTCCCAGTCTATGAAACCAAGG | 9822 |
| hs132666191 | GGCACTCAGGGAGAGTACATGGG | 9823 |
| hs132666542 | AAAGTGACAATTCATTGCAGGGG | 9824 |
| hs132666715 | AGTGATATTATTGTACACGTAGG | 9825 |
| hs132666570 | GATGATGACTTGGACCCTGTGGG | 9826 |
| hs132666296 | GAAATACGTTAACCATTGCAGGG | 9827 |
| hs132666826 | ATTGATAATTGTCTACAGGGTGG | 9828 |
| hs132666833 | CTGAGGTAGCTACAAAACCTGGG | 9829 |
| hs132666343 | TGTAAGTGTGTCTCCCTCTGTGG | 9830 |
| hs132666845 | TTTCAGGAAAGAGTGGCTAGTGG | 9831 |
| hs132666648 | TCATAAGGAAAGAGAATCAGGGG | 9832 |
| hs132666500 | GCAAGGCGAGGAATTTCCGAAGG | 9833 |
| hs132666573 | AGTCATCATCCAATGCATGGTGG | 9834 |
| hs132666183 | ATTGCTGGCATAAAGAGATGAGG | 9835 |
| hs132666750 | GAATGCCATAGGAAGCACTTGGG | 9836 |
| hs132666804 | GCCAGGCTCGAATCAATGAGAGG | 9837 |
| hs132666492 | TCATTGACTCTAGTGTCCCATGG | 9838 |
| hs132666694 | TGTCCTCAAGGATAATGTAGAGG | 9839 |
| hs132666024 | AGAGATCTTTGGAGCCACCAGGG | 9840 |
| hs132666211 | CATCTCAGCAAAGATATCTGAGG | 9841 |
| hs132666177 | ATCTCTTTATGCCAGCAATGAGG | 9842 |
| hs132666407 | TGACATTTTAGAATTTGTGGGGG | 9843 |
| hs132666888 | ATTAGAATTTGTGTATACCAGGG | 9844 |
| hs132666693 | ATGCCTCTACATTATCCTTGAGG | 9845 |
| hs132666065 | AGAGACTTAGAGCCCAACTTGGG | 9846 |
| hs132666179 | AAAGAGATGAGGCTATCCACTGG | 9847 |
| hs132666080 | TCTCATATTTATGAGTCAGAGGG | 9848 |
| hs132666795 | TTTGCATTTGATGAACATTGAGG | 9849 |
| hs132666626 | AAAGAATCAGGAGTCCGTAAGGG | 9850 |
| hs132666603 | AAGAGACAGGTTTCTGACCAGGG | 9851 |
| hs132666905 | GTTCTCGTAAACTACCAGGATGG | 9852 |
| hs132666084 | AGATAGTCTAAATCAAATGAGGG | 9853 |
| hs132666087 | TGAAACTCAAACGTGCCTCAAGG | 9854 |
| hs132666540 | TATGCGAACAAAACTTACATGGG | 9855 |
| hs132666685 | ATTCTGTCTGCTTACCCTGGAGG | 9856 |
| hs132666569 | GGATGATGACTTGGACCCTGTGG | 9857 |
| hs132666806 | GATCTGTGTGATACTCCCATAGG | 9858 |
| hs132666113 | AACTGGGGCTCAGCTCACACTGG | 9859 |
| hs132666730 | ATTATTAAAGATCATCAGGATGG | 9860 |
| hs132666434 | AATAAATGTGGGTCACTAGAGGG | 9861 |
| hs132666173 | CCCAGAACTCTAAGAGATGGTGG | 9862 |
| hs132666582 | GTTTAAATAACTTCCAACCCGGG | 9863 |
| hs132666248 | TTGTAAAGTCCCATCATACAGGG | 9864 |
| hs132666050 | TTACTGACATTCAGTGACATGGG | 9865 |
| hs132666611 | GGAAACTCAACTTTCTCCTGGGG | 9866 |
| hs132666575 | AGTTTAAATTGCCTCCCACAGGG | 9867 |
| hs132666029 | CTACTGATAAATTTAAAGAGCGG | 9868 |
| hs132666719 | TAGATAGTTAAGAAACTATGTGG | 9869 |
| hs132666904 | TCATGTTCTCGTAAACTACCAGG | 9870 |
| hs132666174 | ACTCCCAGAACTCTAAGAGATGG | 9871 |
| hs132666160 | TTGATTTCATGGGAAACAGGTGG | 9872 |
| hs132666698 | TTTATGAACGCTTTACTTGGGGG | 9873 |
| hs132666788 | ATGAAGATGGAGGAAGATTGTGG | 9874 |
| hs132666281 | AAATGTGTAAACTTTGAGAGTGG | 9875 |
| hs132666491 | AGATAATCAAGTGTTGCCATGGG | 9876 |
| hs132666219 | ACTTTGCACTAAGTGAGCCTGGG | 9877 |
| hs132666649 | TTATGACAGCCCCCATAAAGGGG | 9878 |
| hs132666276 | TGTCAGAAGAGCCTCCTGATGGG | 9879 |
| hs132666536 | TTACTTCATACTTCTGCTGGAGG | 9880 |
| hs132666574 | CCAAGTCATCATCCAATGCATGG | 9881 |
| hs132666027 | AATGAATAATTTATCCCTGGTGG | 9882 |
| hs132666483 | ATATAATGTGGTTAAGACCATGG | 9883 |
| hs132666898 | TTAGGTATTATGTAAATCAGTGG | 9884 |
| hs132666831 | GACATTTTACTATACAGCCCAGG | 9885 |
| hs132666485 | AAGACTTTTGGAGCTCACCATGG | 9886 |
| hs132666586 | ACTAGATCCTTTTGGAACCCGGG | 9887 |
| hs132666249 | TTTACAAGTTTTAATTTCAGGGG | 9888 |
| hs132666130 | AGGGAGGCCTGGACACCAGTTGG | 9889 |
| hs132666682 | CTGCATCCTAACTACATAGGAGG | 9890 |
| hs132666797 | GCAGTTGCTGGATGCACAAGCGG | 9891 |
| hs132666505 | AGAAAGTGAGAAATGGCATAGGG | 9892 |
| hs132666652 | TGAATTCTAGAGAATGCTATGGG | 9893 |
| hs132666220 | TCGTTCGTAATTAAATACCCAGG | 9894 |
| hs132666768 | ACATGAAATGACGGAAATGATGG | 9895 |
| hs132666412 | CCAAGGAGTGTGAAAACACGTGG | 9896 |
| hs132666168 | GGACAAAAAGAGTTTTAGCAAGG | 9897 |
| hs132666328 | CATGTGGAAACAAGCAGAGAGGG | 9898 |
| hs132666827 | TTAATTGATAATTGTCTACAGGG | 9899 |
| hs132666836 | AGCTACAGTGTACCTCAGACAGG | 9900 |
| hs132666506 | GAGAAAGTGAGAAATGGCATAGG | 9901 |
| hs132666122 | GTGGAGATAACCAATATACTTGG | 9902 |
| hs132666912 | AGACATTTAATGCAAAACTAGGG | 9903 |
| hs132666841 | GCCTAAATGAGAAGTCCCATTGG | 9904 |
| hs132666523 | TCTCTCTCAAAAATATGCAATGG | 9905 |
| hs132666395 | ACTGCAATCAACGATTCTATGGG | 9906 |
| hs132666370 | ATAGGCTTTTTATTATAACTGGG | 9907 |
| hs132666301 | AGAGCCCTGGGACCCTGCAATGG | 9908 |
| hs132666216 | AATGCCATCTGCAAAGCACTGGG | 9909 |
| hs132666462 | GCTATTACAGATAACATACAAGG | 9910 |
| hs132666812 | ACAAGTCAGGTAAAGAGTCGTGG | 9911 |
| hs132666512 | GGTTTCATAGACTGGGAAGGGGG | 9912 |
| hs132666128 | GTCTGTGAAGATTTTAAGGGAGG | 9913 |
| hs132666111 | AATGGTTTAGACACAGAACTGGG | 9914 |
| hs132666633 | ATATCAACACGAAAGAAGACAGG | 9915 |
| hs132666055 | TATAATTCACTGGTTTGGGTAGG | 9916 |
| hs132666043 | ACCGTCAAGTTTCTGACCCATGG | 9917 |
| hs132666198 | CTGAACCTAAAACTAAGCGTTGG | 9918 |
| hs132666401 | ATTTAATACCAAATTAAGGTAGG | 9919 |
| hs132666062 | GCTTGCAGATAGAGAGACACAGG | 9920 |
| hs132666849 | GTTTGGAGAATGGATTGTGTTGG | 9921 |
| hs132666262 | ATATTTGGTATATATGCCCCAGG | 9922 |
| hs132666035 | CCGTTGAAGTAATTAAGCATAGG | 9923 |
| hs132666846 | TTTTTGTTTTCAGGAAAGAGTGG | 9924 |
| hs132666273 | TTTTAAACATTTATATACACAGG | 9925 |
| hs132666275 | ATGTCAGAAGAGCCTCCTGATGG | 9926 |
| hs132666214 | CATGAAGACATTTAAATCAGAGG | 9927 |
| hs132666819 | ATATTCAGTGAAATATTAGTGGG | 9928 |
| hs132666600 | AGAGATATAAAAATGATGCCAGG | 9929 |
| hs132666748 | AGAATAATACTGAATGCCATAGG | 9930 |
| hs132666439 | AATTGAAGACAAGCTCTGAAAGG | 9931 |
| hs132666227 | TTATGTGAGTTGTTAAAGTGGGG | 9932 |
| hs132666175 | TCTCTCCAATATTCTGCCAGTGG | 9933 |
| hs132666372 | TCAGTGGTTTACACAGCACCTGG | 9934 |
| hs132666398 | AAAATATTTTGACGTCTGGTAGG | 9935 |
| hs132666119 | ATATACTTGGCAGAGCCTCTAGG | 9936 |
| hs132666178 | CTTTATGCCAGCAATGAGGATGG | 9937 |
| hs132666097 | TTTCTGCAGTGAATTGGAGGTGG | 9938 |
| hs132666046 | AACTTGAAATTAAGAGACTAGGG | 9939 |
| hs132666875 | TTAGCCAAGCTGACACAACCAGG | 9940 |
| hs132666548 | GACTGGATAATTAAAGAGTAGGG | 9941 |
| hs132666890 | ATATGCTGCTTAAAAACATACGG | 9942 |
| hs132666298 | TTAACCATTGCAGGGTCCCAGGG | 9943 |
| hs132666225 | TTCAATGCCATATTCAACGTTGG | 9944 |
| hs132666094 | GTTGGAGAAAGATTTCTACTAGG | 9945 |
| hs132666210 | GATATCTTTGCTGAGATGCATGG | 9946 |
| hs132666236 | GGAAAGTAAGTTAAGCACTAAGG | 9947 |
| hs132666232 | GGACTGATTCCAAAGGTGTAGGG | 9948 |
| hs132666546 | TTAAAGAGTAGGGAAGCATTGGG | 9949 |
| hs132666348 | TGTCTCCCTCTGTGGAAGGGGGG | 9950 |
| hs132666840 | CCTAAATGAGAAGTCCCATTGGG | 9951 |
| hs132666044 | GATTGAGTTGTTATTTATCGAGG | 9952 |
| hs132666794 | AGTAGGTATGGTTAATTGGAAGG | 9953 |
| hs132666327 | GAGGGACAAGTTTTCCCAGAAGG | 9954 |
| hs132666894 | TTACAATAACTGGAATGAAAGGG | 9955 |
| hs132666300 | CAATGGTTAACGTATTTCACAGG | 9956 |
| hs132666322 | GAAAGGGGAAGAGTAGAGAGAGG | 9957 |
| hs132666709 | AATAGATCTAGTTTTACCCTAGG | 9958 |
| hs132666860 | CTGTTTGGAGAACTGTTGGAGGG | 9959 |
| hs132666623 | TGTAGAGTATGAGAGGGTAAAGG | 9960 |
| hs132666734 | GAACAATTCCAGGAAGAGCTAGG | 9961 |
| hs132666417 | AACTGCATGACCCAGAGGATGGG | 9962 |
| hs132666042 | ACCATGGGTCAGAAACTTGACGG | 9963 |
| hs132666030 | CCTATGCTTAATTACTTCAACGG | 9964 |
| hs132666585 | GATCCTTTTGGAACCCGGGTTGG | 9965 |
| hs132666230 | ATACATAAGTTCATTAGCAATGG | 9966 |
| hs132666760 | AGTAAATCAATTAGAAAGGAGGG | 9967 |
| hs132666351 | ATCTTCCCCCCTTCCACAGAGGG | 9968 |
| hs132666364 | CAAAACCTCTTTAATTAGAGAGG | 9969 |
| hs132666625 | GAAAGAATCAGGAGTCCGTAAGG | 9970 |
| hs132666167 | AGGAAATCAGAGATGTTTGAAGG | 9971 |
| hs132666085 | GAGATAGTCTAAATCAAATGAGG | 9972 |
| hs132666511 | TCTATGAAACCAAGGTAAGATGG | 9973 |
| hs132666541 | GTATGCGAACAAAACTTACATGG | 9974 |
| hs132666929 | GCTTTTATCTGCAAATGAAGTGG | 9975 |
| hs132666604 | ATTCTTAGTAAAGTCTTCCCTGG | 9976 |
| hs132666337 | ATTACAGAATATTATTAAAGGGG | 9977 |
| hs132666068 | GGATTTTATTCCATTGGAGGAGG | 9978 |
| hs132666017 | TTAATACTAATAGAGGTGTCAGG | 9979 |
| hs132666837 | ATCTTTCTGTATGTGCCCAATGG | 9980 |
| hs132666064 | GTTGGGCTCTAAGTCTCTACAGG | 9981 |
| hs132666438 | ATTGAAGACAAGCTCTGAAAGGG | 9982 |
| hs132666245 | CCATAAATAATAACAAATTGAGG | 9983 |
| hs132666507 | ACACGTGAGAAAGTGAGAAATGG | 9984 |
| hs132666235 | AGCACTAAGGAATATATTAGTGG | 9985 |
| hs132666189 | GTACTCTCCCTGAGTGCCAATGG | 9986 |
| hs132666120 | TGGTTATCTCCACACAAAGAAGG | 9987 |
| hs132666579 | AGAGAAGTCAACATGCTTGTGGG | 9988 |
| hs132666423 | GCACAGAAGTGTGTTTCAAAGGG | 9989 |
| hs132666803 | GGAGTCAGTCACTTAGAGCCAGG | 9990 |
| hs132666140 | CCTTTTATACAAAGATCGTATGG | 9991 |
| hs132666686 | TTCTGTCTGCTTACCCTGGAGGG | 9992 |
| hs132666154 | TGAAAGGGAAGAACTCCAACAGG | 9993 |
| hs132666791 | TATGTGTGCAATGTATAAGTAGG | 9994 |
| hs132666034 | CGTTGAAGTAATTAAGCATAGGG | 9995 |
| hs132666449 | CTGTGGACATATTTTCTCAAGGG | 9996 |
| hs132666514 | TTGGTTTCATAGACTGGGAAGGG | 9997 |
| hs132666702 | TAGCAACCAGGCAGAATGAATGG | 9998 |
| hs132666583 | CTTCCAACCCGGGTTCCAAAAGG | 9999 |
| hs132666315 | ATTAAAATGTCTTCTCACTTCGG | 10000 |
| hs132666132 | TATCAGAACTACGTGCCAACTGG | 10001 |
| hs132666023 | AAGAGATCTTTGGAGCCACCAGG | 10002 |
| hs132666238 | GGATAGAGTCTGATAACATTCGG | 10003 |
| hs132666868 | TGATGGAGTGAAAAGCATAATGG | 10004 |
| hs132666493 | AGTGCAGCTTCCATAGACTTTGG | 10005 |
| hs132666576 | AAGTTTAAATTGCCTCCCACAGG | 10006 |
| hs132666621 | AGAAGGGAGTGAAGCTGGAGAGG | 10007 |
| hs132666610 | TGGAAACTCAACTTTCTCCTGGG | 10008 |
| hs132666435 | TTTATTTATCATAAGGACATAGG | 10009 |
| hs132666538 | CCAGCAGAAGTATGAAGTAAAGG | 10010 |
| hs132666411 | AGGAATTAGTTATAAAGCCAAGG | 10011 |
| hs132666628 | GAACTATCTGTATTCCCTTACGG | 10012 |
| hs132666345 | GTGTGTCTCCCTCTGTGGAAGGG | 10013 |
| hs132666558 | TCTTCATGACAGTTTATGCTGGG | 10014 |
| hs132666477 | GGAAATTAAGGGAAATTCCAAGG | 10015 |
| hs132666090 | TGAGGCACGTTTGAGTTTCAGGG | 10016 |
| hs132666814 | AAAGAGTCGTGGGTGACAACTGG | 10017 |
| hs132666047 | CTAGTCTCTTAATTTCAAGTTGG | 10018 |
| hs132666848 | ACACAATCCATTCTCCAAACAGG | 10019 |
| hs132666725 | GTAAATAAGGATACCCTAATGGG | 10020 |
| hs132666614 | AGTTTCCAATTATTAATTCAGGG | 10021 |
| hs132666897 | CAGTGGATATATTAAAATGATGG | 10022 |
| hs132666671 | ATTAGGTCCTTCAAATAACTTGG | 10023 |
| hs132666363 | ACTTACCTCTCTAATTAAAGAGG | 10024 |
| hs132666326 | CCTCTCTGCTTGTTTCCACATGG | 10025 |
| hs132666096 | CTGCAGTGAATTGGAGGTGGTGG | 10026 |
| hs132666815 | TGGTAGTGTGTCACAGAAGCTGG | 10027 |
| hs132666747 | GAAGGTAGACATAAAATTAGTGG | 10028 |
| hs132666589 | AAGTACACGTCAGATTGAAATGG | 10029 |
| hs132666690 | TGGTCTGTTTTTTCCCTCCAGGG | 10030 |
| hs132666822 | GCTTCAGATGATACAAAAACGGG | 10031 |
| hs132666883 | ATAATTCCAAAGTAAATCACTGG | 10032 |
| hs132666390 | ATCATTCAATTTGTATGCAAGGG | 10033 |
| hs132666522 | TTTTGATCTCACTTGAGAGTTGG | 10034 |
| hs132666067 | TATTCTTGTTCCTCCTCCAATGG | 10035 |
| hs132666019 | GCTGCTATTAATACTAATAGAGG | 10036 |
| hs132666737 | ATAGGCTTCCTTTGTTTTGACGG | 10037 |
| hs132666103 | TTGTATGTTTCCTTTTAAAACGG | 10038 |
| hs132666079 | CTGACTCATAAATATGAGATAGG | 10039 |
| hs132666153 | TTTGGACTCTTCTCACCTGTTGG | 10040 |
| hs132666508 | GATAAAGTAAAAGCCACAATTGG | 10041 |
| hs132666488 | TTTTCAGCATCAAAAATAAGTGG | 10042 |
| hs132666231 | ATAATGTGTCCCTACACCTTTGG | 10043 |
| hs132666141 | CATACGATCTTTGTATAAAAGGG | 10044 |
| hs132666641 | TTAACCTGCCCAGGAATAGATGG | 10045 |
| hs132666865 | TGTTTATAGACAACGCTTAGTGG | 10046 |
| hs132666673 | ACTAAAATCTGAAAGTTGTATGG | 10047 |
| hs132666389 | AACATACTAAGTATATAATTCGG | 10048 |
| hs132666879 | AAAGGCCCAGTGATTTACTTTGG | 10049 |
| hs132666013 | TATTTTTTGCTGTCTTAGGTTGG | 10050 |
| hs132666292 | CTTTCAGGTTTCTAAGGATGAGG | 10051 |
| hs132666810 | TCCTTCTGCCTTATTCCTATGGG | 10052 |
| hs132666830 | ACTGAACGAAGATAGCAAAAAGG | 10053 |
| hs132666642 | TCTATTCCTGGGCAGGTTAAGGG | 10054 |
| hs132666241 | GCTAAAGGAGATTTTAAGCTAGG | 10055 |
| hs132666243 | AAGATATACTTGAAGAATAGAGG | 10056 |
| hs132666161 | GCTTGAATTGTGTAAGAATTGGG | 10057 |
| hs132666552 | GAGAGGCATGGAAGACTTATGGG | 10058 |
| hs132666018 | GCAGCACTATTTTCCCTGTTAGG | 10059 |
| hs132666118 | AGAGGCTCTGCCAAGTATATTGG | 10060 |
| hs132666529 | GCATGTAGACTTAAACCTTCTGG | 10061 |
| hs132666127 | TCTGTCTGTGAAGATTTTAAGGG | 10062 |
| hs132666467 | TACACAGTACAGTAATTTAGTGG | 10063 |
| hs132666266 | ATTTTTATGGGAAATTCAACTGG | 10064 |
| hs132666706 | TCATGTAGCAGTTATTTTAAGGG | 10065 |
| hs132666447 | GAGAAAATATGTCCACAGTTTGG | 10066 |
| hs132666402 | TTAATCATTTCAAAATTCATTGG | 10067 |
| hs132666807 | GTGATACTCCCATAGGAATAAGG | 10068 |
| hs132666478 | TGAGGATTTTAGGAAATTAAGGG | 10069 |
| hs132666414 | CCACGTGTTTTCACACTCCTTGG | 10070 |
| hs132666650 | CTTATGACAGCCCCCATAAAGGG | 10071 |
| hs132666307 | TTAAAATGTTCATTAAGAATTGG | 10072 |
| hs132666906 | GGATGGATCCCATCTATTATAGG | 10073 |
| hs132666742 | ACGAATGTAATGTAATTTACTGG | 10074 |
| hs132666909 | AGAAGTACACCTATAATAGATGG | 10075 |
| hs132666726 | TATTTACCCAAAACAATTAAGGG | 10076 |
| hs132666470 | TTGTATACTTTGTACCCTAATGG | 10077 |
| hs132666724 | GGTAAATAAGGATACCCTAATGG | 10078 |
| hs132666736 | ACATAAAGAAGTGTAATAATAGG | 10079 |
| hs132666680 | ATTTATCCTCCTATGTAGTTAGG | 10080 |
| hs132666336 | AATTACAGAATATTATTAAAGGG | 10081 |
| hs132666163 | TATTTTATGTTGCTTCCATTTGG | 10082 |
| hs132666086 | GATTGCTCACTTCTACTTAGAGG | 10083 |
| hs132666176 | GCTATCCACTGGCAGAATATTGG | 10084 |
| hs132666379 | TTTGAGACCATGCAAATATAAGG | 10085 |
| hs132666524 | TGAGAGAGAGAAATATGATATGG | 10086 |
| hs132666445 | CTTTTGTGTGTACCTCAAAATGG | 10087 |
| hs132666052 | CCTTAAATACTCTCCTACTATGG | 10088 |
| hs132666665 | GTAATGACACTTTTGCCTATTGG | 10089 |
| hs132666799 | GATATAAAGCTTTAATTTGAGGG | 10090 |
| hs132666039 | CCTTAAATTGGGTACTTAAAGGG | 10091 |
| hs132666359 | TATCTTTCTGGTATTTTCTTGGG | 10092 |
| hs132666032 | TTACTTCAACGGATATTCTATGG | 10093 |
| hs132666440 | TTATCTGCATAACGAAAATATGG | 10094 |
| hs132666720 | CATAGTTTCTTAACTATCTAAGG | 10095 |
| hs132666105 | AGACATTTGACCGTTTTAAAAGG | 10096 |
| hs132666722 | TTAAATCCCTTAATTGTTTTGGG | 10097 |
| hs132666608 | ACCATCCCTGAATTAATAATTGG | 10098 |
| hs132666728 | GATACCCTAATGGGTAAAATTGG | 10099 |
| hs132666501 | GCTATGTTGTAATAAGTTAAAGG | 10100 |
| hs132666212 | AATGTCTTCATGCTTTAAATTGG | 10101 |
| hs132666853 | GCTCTGTTAATTTCTGTATTAGG | 10102 |
| hs132666106 | ATTCTCGTCTCTGTTCTTTATGG | 10103 |
| hs132666400 | ATAATCTTCCTACCTTAATTTGG | 10104 |
| hs132665858 | AGCCTGCCGAGTGAGAGTGTGGG | 10105 |
| hs132665800 | TACAGCTGAATGGAACCAGGTGG | 10106 |
| hs132665870 | ACTGGCTTGATGATTTCCAGCGG | 10107 |
| hs132665656 | TTAGAATAACTGGAACAATGTGG | 10108 |
| hs132665886 | GAAAATCTGGAGCTTCTGTGAGG | 10109 |
| hs132665669 | AGGGCCAACAAGATATCACAAGG | 10110 |
| hs132665649 | GTTATCTTCTCCTATCAGAGGGG | 10111 |
| hs132665639 | TAAATATCAAGAAAGACAGGAGG | 10112 |
| hs132665801 | TGACTATGTATTAAGCCACCTGG | 10113 |
| hs132665592 | AATACTGGAGTACAGATGGGAGG | 10114 |
| hs132665760 | GAAGCAAAGAATACAACACAGGG | 10115 |
| hs132665564 | ATTACTAATAAGTATATAGGCGG | 10116 |
| hs132665740 | TGAACATGGCAATTTCGAGGAGG | 10117 |
| hs132665715 | TACTTTGCAAGATAGACCTGTGG | 10118 |
| hs132665863 | TAGAAGAAAATATAGACTCGAGG | 10119 |
| hs132665981 | AAAATTATACCATCTCAGAAGGG | 10120 |
| hs132665914 | GCTCGTGTCATGAACTTGAGTGG | 10121 |
| hs132665651 | AGTAGAATGAGAACACTATGTGG | 10122 |
| hs132666006 | GAATCACAGATAACAAAGCACGG | 10123 |
| hs132665889 | AATCCCCTTGTGATCAAAGGAGG | 10124 |
| hs132665696 | TTTGAGGACGAACGTATGGGTGG | 10125 |
| hs132665892 | ATAGCCTCCTTTGATCACAAGGG | 10126 |
| hs132665881 | CCATAACGACTTGATAGGGAGGG | 10127 |
| hs132665939 | GATTTGCATTTTATCAATAGCGG | 10128 |
| hs132665987 | CCTTTCTCAGTATTTACATGTGG | 10129 |
| hs132665764 | AGAGGTGCTAATAAAGAAGGAGG | 10130 |
| hs132665849 | CAGTCATTCTAAAGTAGTGGGGG | 10131 |
| hs132665992 | ATATCTTATAATATATGACAGGG | 10132 |
| hs132665668 | ATTTAACTATTAATTCAGCAGGG | 10133 |
| hs132665897 | TGATACTAAAAGAATGTGAGAGG | 10134 |
| hs132665624 | GGAACGTGCACAATCATGGATGG | 10135 |
| hs132665925 | ATACATTGTATTTAGCCTGAGGG | 10136 |
| hs132665824 | GAAAATATACTTAGGATGCACGG | 10137 |
| hs132665876 | GGTAATCAAGTGCAGATCCGAGG | 10138 |
| hs132665799 | ATATACAGCTGAATGGAACCAGG | 10139 |
| hs132665652 | TTATGATGATAACATTTAGAGGG | 10140 |
| hs132665794 | AAAGCTAATGATAAAGACCTGGG | 10141 |
| hs132665968 | ATTTAGATGTGATCTTCCCATGG | 10142 |
| hs132665709 | GTGACTGTGTATCAACAAAGAGG | 10143 |
| hs132665915 | AATAACCTATATAAACCGACAGG | 10144 |
| hs132665810 | GGAAGAGGCTATCCTTCACCTGG | 10145 |
| hs132665839 | GCCAGAAGTCATTAACTGCAAGG | 10146 |
| hs132665591 | TAAAATACTGGAGTACAGATGGG | 10147 |
| hs132665997 | GTAACTGAACAATATATGAAAGG | 10148 |
| hs132665612 | GGTGCTTTGAAATTTTCAAGTGG | 10149 |
| hs132665891 | TAGCCTCCTTTGATCACAAGGGG | 10150 |
| hs132665803 | GATGAGCCTCAACCAGGTGAAGG | 10151 |
| hs132665853 | AGTCATAGTGCTGTTTTCGAGGG | 10152 |
| hs132665808 | GCCTCTTCCACTAAGGCAGGTGG | 10153 |
| hs132665733 | GAGTATTCAGATGATACCCTTGG | 10154 |
| hs132665711 | ATACACAGTCACTTAAAATGAGG | 10155 |
| hs132665879 | ATGACCATAACGACTTGATAGGG | 10156 |
| hs132665661 | ACATGCTCTGCTGTTGCTAGGGG | 10157 |
| hs132665697 | TCATTTGAGGACGAACGTATGGG | 10158 |
| hs132665617 | GCTAATTGGAAAGGGATCCTGGG | 10159 |
| hs132665941 | TAAAAGTTCCTGTTATCATGTGG | 10160 |
| hs132665934 | GAGAGTTCAGGTTGATAAAGGGG | 10161 |
| hs132665820 | AATTATTTCAAGCTTTTGTGGGG | 10162 |
| hs132665610 | ATTTTACATGGTGCTCTGAAGGG | 10163 |
| hs132665572 | TCAAAGTACTCCTATTTCCAAGG | 10164 |
| hs132665831 | TGATAGCAGGAGAGCCCACAGGG | 10165 |
| hs132665899 | ATTTAAAAGCTGACTTCTGGGGG | 10166 |
| hs132665851 | AGTAGTGGGGGTACAAAAGAGGG | 10167 |
| hs132665744 | GAGAAAGATAATATTGTCAAGGG | 10168 |
| hs132665871 | CTGGCTTGATGATTTCCAGCGGG | 10169 |
| hs132665679 | ATAGTTTTTATAGTAAAATGGGG | 10170 |
| hs132665908 | ACTGGCTAAAAGCATTTGAAAGG | 10171 |
| hs132665920 | TTTCTTACTACATCACCTGTCGG | 10172 |
| hs132665994 | ATATAATAGATACCATACTTAGG | 10173 |
| hs132665862 | TGCTTTCCCACACTCTCACTCGG | 10174 |
| hs132665750 | ATAATTTAGAATCCGAGTAGTGG | 10175 |
| hs132665607 | TGAAAGATACACATTTTACATGG | 10176 |
| hs132665749 | CATGTTTAGAAGCCACTACTCGG | 10177 |
| hs132665846 | AGTCAGTCATTCTAAAGTAGTGG | 10178 |
| hs132665995 | ACAATTGTGCAAGCTTGCTAAGG | 10179 |
| hs132665710 | GTATCAACAAAGAGGAACATAGG | 10180 |
| hs132665965 | TCGTATTTGTAAAAATACAATGG | 10181 |
| hs132665692 | TGAAAATCGCCATTTCTCCTGGG | 10182 |
| hs132665834 | TAGTTATTTCAGCATCCCTGTGG | 10183 |
| hs132665647 | GAGTTATCTTCTCCTATCAGAGG | 10184 |
| hs132665797 | AATCTGGATATACAGCTGAATGG | 10185 |
| hs132665762 | GAGGAACAGAGGTCTGAAAGAGG | 10186 |
| hs132665833 | AGTTATTTCAGCATCCCTGTGGG | 10187 |
| hs132665700 | ATGAACTTCTGTTTTCAGGTAGG | 10188 |
| hs132665887 | TCAAATCCCCTTGTGATCAAAGG | 10189 |
| hs132665825 | GAAAGCTTATGAAGAACTGCTGG | 10190 |
| hs132665726 | TTCCCATGTTTTCAAGACCAAGG | 10191 |
| hs132665636 | TTTCTCCTCTAAGATGCTCAGGG | 10192 |
| hs132665874 | AAAAAGTTGATTCTGAAAGAGGG | 10193 |
| hs132665802 | CCCAGAGATGAGCCTCAACCAGG | 10194 |
| hs132665769 | GTTAGAGAAGATACAGAAAGAGG | 10195 |
| hs132665927 | TTTACCTTGATGTTTCCCTCAGG | 10196 |
| hs132665704 | TTCTGTGAGTTAATGGGAGACGG | 10197 |
| hs132665648 | AGTTATCTTCTCCTATCAGAGGG | 10198 |
| hs132665943 | TAAGACAGCTTTGATCAGGCAGG | 10199 |
| hs132665727 | TCCCATGTTTTCAAGACCAAGGG | 10200 |
| hs132665686 | GAGCACATATTTCAGTTTGTCGG | 10201 |
| hs132665969 | TGAAAAGTAATTTTATCCATGGG | 10202 |
| hs132665984 | CCACATGTAAATACTGAGAAAGG | 10203 |
| hs132665741 | TTATGAACATGGCAATTTCGAGG | 10204 |
| hs132665807 | ATAGCCTCTTCCACTAAGGCAGG | 10205 |
| hs132665844 | GGAATTGTCTCTGTTAGGTGAGG | 10206 |
| hs132665979 | ATAAAACTTCCTATTTGTGAAGG | 10207 |
| hs132665867 | GTGTTTACTACCCAGAGTATAGG | 10208 |
| hs132665945 | ATTATTGATCTGATTCTCACAGG | 10209 |
| hs132665955 | TTTTTAGGCTGCTTGAAAAAGGG | 10210 |
| hs132665590 | GTAAAATACTGGAGTACAGATGG | 10211 |
| hs132665772 | TAAGTTACTCTTCAGTCAAAGGG | 10212 |
| hs132665845 | GAATTGTCTCTGTTAGGTGAGGG | 10213 |
| hs132665916 | GGTTATTTTAAACTCATGGCAGG | 10214 |
| hs132665938 | ATTTGCATTTTATCAATAGCGGG | 10215 |
| hs132665662 | TGTTGCTAGGGGTATTTCAATGG | 10216 |
| hs132665586 | AAGACTTCATTTACATCCACAGG | 10217 |
| hs132665875 | AACTTTTTACCAACTCCCGCTGG | 10218 |
| hs132665688 | TGATGAGAGTACCTTGTTCGTGG | 10219 |
| hs132665608 | GTATCTTTCAGCTAAGAGATAGG | 10220 |
| hs132665766 | AGAAAGAGGAGGCTTTTAAGAGG | 10221 |
| hs132665972 | ACAGGCAGAGTAAATAAAATGGG | 10222 |
| hs132665619 | TTTTACCTGCTAATTGGAAAGGG | 10223 |
| hs132666003 | GATTCTTCTGTGACTGACTAGGG | 10224 |
| hs132665558 | TGACAGAGTGAAACATCTTAAGG | 10225 |
| hs132665743 | GATAATATTGTCAAGGGCAGAGG | 10226 |
| hs132665780 | TTGGTTTGTGTGTTAGGCATGGG | 10227 |
| hs132665616 | AGGATCCCTTTCCAATTAGCAGG | 10228 |
| hs132665729 | GGTCTTGAAAACATGGGAAAGGG | 10229 |
| hs132665722 | GCACTTTAAGAGAGCATTGTGGG | 10230 |
| hs132665948 | TTGTTTGCTGTGATTTTTGAGGG | 10231 |
| hs132665898 | ATATTCTAGGACACAATCTTGGG | 10232 |
| hs132665991 | AATATCTTATAATATATGACAGG | 10233 |
| hs132665792 | CTAAAAGATGAAAACACAACCGG | 10234 |
| hs132665565 | ATACTTATTAGTAATTTCAATGG | 10235 |
| hs132665765 | TTAAGAGGTGCTAATAAAGAAGG | 10236 |
| hs132665674 | AATGGTCTCTGACAGTCTATAGG | 10237 |
| hs132665878 | TCTGGCTTTGGGCTCTTCCTCGG | 10238 |
| hs132665635 | AAGCACCCTGAGCATCTTAGAGG | 10239 |
| hs132665885 | CCCTCCCTATCAAGTCGTTATGG | 10240 |
| hs132665933 | TGAGAGTTCAGGTTGATAAAGGG | 10241 |
| hs132665576 | GTTCAAAATGTAACTCTTAAGGG | 10242 |
| hs132665940 | ATAGGTAACCACATGATAACAGG | 10243 |
| hs132665905 | ACAAGGAAAAAATTATGCTTAGG | 10244 |
| hs132665937 | ATAAAATGCAAATCGTTCATAGG | 10245 |
| hs132665837 | TATTTTCTCATTGAAGACTTTGG | 10246 |
| hs132665755 | AGAATTATAAATACTAAAATGGG | 10247 |
| hs132665950 | TTTCAAGTATACTTCTTTGAAGG | 10248 |
| hs132665784 | ACTTGAACGTGAAGTGGCTTTGG | 10249 |
| hs132665713 | CTATCTTGCAAAGTAAAAACTGG | 10250 |
| hs132665928 | ATTTCATGTCCCATTTTAGTAGG | 10251 |
| hs132665909 | TTAGCCAGTTAGCATATCATAGG | 10252 |
| hs132665659 | AGACATGCTCTGCTGTTGCTAGG | 10253 |
| hs132665561 | GTTGTAGCAAGTCATGAAAATGG | 10254 |
| hs132665895 | GAGCACATGGTAACGCAAATTGG | 10255 |
| hs132665819 | GTGAATGATGGGGAAAGTTTAGG | 10256 |
| hs132665673 | CATTCCTTGTGATATCTTGTTGG | 10257 |
| hs132665975 | CATGCATCTTTGTATATGATTGG | 10258 |
| hs132665877 | GATGACCATAACGACTTGATAGG | 10259 |
| hs132665854 | GCTGTTTTCGAGGGTAAAATTGG | 10260 |
| hs132665776 | TCTGCAGTTGCATTTAAGATAGG | 10261 |
| hs132665919 | CATCACCTGTCGGTTTATATAGG | 10262 |
| hs132665869 | GAGAAATTGGAAGCATTAACTGG | 10263 |
| hs132665684 | TATGTGCTCATTCTACTTAAAGG | 10264 |
| hs132665868 | ATAACAGAGATAGGAGAAATTGG | 10265 |
| hs132665663 | TCAATGGATATTATTCAAAAAGG | 10266 |
| hs132665912 | TTTTAGGCTTTCATTAAAAATGG | 10267 |
| hs132665911 | TTTTAATGAAAGCCTAAAAATGG | 10268 |
| hs132665634 | ATATAACATTTACCTTTTAATGG | 10269 |
| hs132665568 | TATTTCTGCTAAATTATTTATGG | 10270 |
| hs132665428 | AAGAATGTATGTGTCTTACGTGG | 10271 |
| hs132665421 | ACTTCTGAGGAAACTATACGTGG | 10272 |
| hs132665475 | GCAGATACCAGGGAACACTGGGG | 10273 |
| hs132665275 | CTGTTGTTGTGGGAAATGAGTGG | 10274 |
| hs132665500 | GTGATAATACAGTTTCTAGGGGG | 10275 |
| hs132665195 | AAAACATTTCAAAAAAAACGTGG | 10276 |
| hs132665200 | ATATGCTGCCTGCTCAACAAGGG | 10277 |
| hs132665114 | CATAATGCTACTCAGGATGGCGG | 10278 |
| hs132665473 | AGATACCAGGGAACACTGGGGGG | 10279 |
| hs132665124 | CTAAGTATAAAAGAAGTCCGTGG | 10280 |
| hs132665072 | GGAGTACCTACTATGTACCATGG | 10281 |
| hs132665185 | TCTCTTGTGTGAAAATTAGGGGG | 10282 |
| hs132665484 | AGTCGTTGAGGTAATAACGTTGG | 10283 |
| hs132665207 | ATTCTATTAGATACTGCCAGTGG | 10284 |
| hs132665240 | AATAAATATATTGCACCCAGAGG | 10285 |
| hs132665441 | AGCCCAAAAGTACAGTTGAGTGG | 10286 |
| hs132665248 | TGAAGTACTTGCATGGAAGGTGG | 10287 |
| hs132665144 | GTGAAACGTCAACATGAACCAGG | 10288 |
| hs132665289 | AATCAAACTGCCCAGTACTGAGG | 10289 |
| hs132665084 | AGATCTCTGTAGGTACATGAGGG | 10290 |
| hs132665176 | CATCTATAATAGCAAAAAGGGGG | 10291 |
| hs132665241 | TTATTCACAGACTATTTCAGGGG | 10292 |
| hs132665423 | GAGGAACAGCCATTTACATGAGG | 10293 |
| hs132665288 | GCATTATACTAAGTGTCATGTGG | 10294 |
| hs132665263 | ACCTGTGAAGACAAGTTCTGAGG | 10295 |
| hs132665362 | ACTTAATGAGGGCAAGTGAGAGG | 10296 |
| hs132665143 | AGCAGTTAGAAGTCCATGAAGGG | 10297 |
| hs132665530 | TTATTACAAAACAGGAATGACGG | 10298 |
| hs132665368 | TAAGCTTTGGATCATAACCCTGG | 10299 |
| hs132665121 | ATCTCATTAAAAATATATCAGGG | 10300 |
| hs132665474 | CAGATACCAGGGAACACTGGGGG | 10301 |
| hs132665098 | GTGCAAGTTAGCAAAACCACAGG | 10302 |
| hs132665298 | GCTACTCATTCATGATGAAGGGG | 10303 |
| hs132665127 | TATGTAGCTCCTGTAGGCCACGG | 10304 |
| hs132665479 | GCTAACTTTGGCAGATACCAGGG | 10305 |
| hs132665352 | ACTGATGCCTTTCATGACCAAGG | 10306 |
| hs132665284 | CCAAAAGGAAGAGCCTCTGTGGG | 10307 |
| hs132665166 | TATATTGCAACATTTTTTGGGGG | 10308 |
| hs132665123 | ATGTAAAAAGAATATATACATGG | 10309 |
| hs132665106 | AATAGAACATTAGACTATCAGGG | 10310 |
| hs132665077 | CCATACATAGCATGAAAGCATGG | 10311 |
| hs132665432 | TCCATAGCCTAAGCTTGGGAAGG | 10312 |
| hs132665076 | AAATGACATAGCATATTCCATGG | 10313 |
| hs132665399 | TGAAAGTACAAAATTTACCATGG | 10314 |
| hs132665268 | AAAAGTAATACCCCTAGTGAGGG | 10315 |
| hs132665457 | TTAATGGGGAAACAGAGCTATGG | 10316 |
| hs132665465 | TCTTTACTGGTAGAACAAAAGGG | 10317 |
| hs132665419 | ATGAGTGGTTTATACAAGAGTGG | 10318 |
| hs132665397 | ACCATGGTTACAATGTAGAAGGG | 10319 |
| hs132665129 | TTAGAATATGTAGCTCCTGTAGG | 10320 |
| hs132665053 | AAGCTCTGATGGGAGCTGGAAGG | 10321 |
| hs132665250 | GCAGTTCTGAAGTACTTGCATGG | 10322 |
| hs132665155 | TGTATGGTTGACCTTTCTCAGGG | 10323 |
| hs132665435 | GAATCTTCCTTCCCAAGCTTAGG | 10324 |
| hs132665395 | TCCCTTCTACATTGTAACCATGG | 10325 |
| hs132665066 | AACCCAGAAAACTTTATCAAGGG | 10326 |
| hs132665221 | TTCTCATTTTAATTGAAGAGAGG | 10327 |
| hs132665142 | AAGCAGTTAGAAGTCCATGAAGG | 10328 |
| hs132665311 | AGATTTTGTCAGATGGGTGAGGG | 10329 |
| hs132665051 | AAAGTGCTCAAAGCTCTGATGGG | 10330 |
| hs132665364 | GGAAACTGTTAACTTAATGAGGG | 10331 |
| hs132665074 | ACATAGTAGGTACTCCAGAATGG | 10332 |
| hs132665177 | CTATAATAGCAAAAAGGGGGAGG | 10333 |
| hs132665105 | ACATTAGACTATCAGGGCAGAGG | 10334 |
| hs132665445 | CCCTCTGAAATACACCTGAAGGG | 10335 |
| hs132665401 | TATGCTGAGAAGACAGGATAGGG | 10336 |
| hs132665402 | ATATGCTGAGAAGACAGGATAGG | 10337 |
| hs132665407 | GCTTCATAGACATTTAGAAACGG | 10338 |
| hs132665418 | TAGTGGCATAATAATATGAGTGG | 10339 |
| hs132665203 | CCTATAGGAAATGTCTCCACTGG | 10340 |
| hs132665302 | GTCTGTATACAAATGTGGCATGG | 10341 |
| hs132665332 | CCTTTCACCATAATTTCTAGAGG | 10342 |
| hs132665119 | AAATATATCAGGGAAGAAGGAGG | 10343 |
| hs132665184 | CTCTCTTGTGTGAAAATTAGGGG | 10344 |
| hs132665345 | TGGAGTTTTAGGACAACCTAGGG | 10345 |
| hs132665245 | AGTAATGATCCATTTCCTCTGGG | 10346 |
| hs132665507 | AACCAAATACAGTCAGTCATGGG | 10347 |
| hs132665495 | ATTTCTGATTAATTCAGTGTAGG | 10348 |
| hs132665151 | TCAATCCAGCATCAGTCTATCGG | 10349 |
| hs132665194 | ATCTCACATGATTATAAATGTGG | 10350 |
| hs132665216 | GCTTTAAAATACTTGGGCAGGGG | 10351 |
| hs132665350 | TGAAAGGCCTTGGTCATGAAAGG | 10352 |
| hs132665381 | AAAGAACTGGAACATCCATAAGG | 10353 |
| hs132665313 | GCAACCAGATTTTGTCAGATGGG | 10354 |
| hs132665283 | TCTGTGGGTGTGTGTGAGCACGG | 10355 |
| hs132665104 | CATTAGACTATCAGGGCAGAGGG | 10356 |
| hs132665431 | TATCTCCATAGCCTAAGCTTGGG | 10357 |
| hs132665491 | TGGTAAGTAAAACTACATGCAGG | 10358 |
| hs132665270 | AATTTATATTGCCCTCACTAGGG | 10359 |
| hs132665130 | TTGCTTCCTTAAGCATTAGGAGG | 10360 |
| hs132665394 | ATACTTTGCACCATCAAATGAGG | 10361 |
| hs132665260 | TTTTGCTGAACTTTAAGTGAGGG | 10362 |
| hs132665357 | GTAGCTGTGCATATTGAAAAGGG | 10363 |
| hs132665272 | TTGAAAATAAAAACGCAAAAGGG | 10364 |
| hs132665542 | TAGGGATTTGATAAACACATAGG | 10365 |
| hs132665297 | GGCTACTCATTCATGATGAAGGG | 10366 |
| hs132665370 | AAAATGTCTCTAGTAAAAAGAGG | 10367 |
| hs132665040 | CTTTCAGTCTGTGGGTTCAGGGG | 10368 |
| hs132665159 | AACTACAGTGAAGTACCAATAGG | 10369 |
| hs132665073 | TAGGTACTCCAGAATGGTAATGG | 10370 |
| hs132665161 | GCAGTATCCGTTAAAATCTAGGG | 10371 |
| hs132665412 | TTTAGAATGAAATTCCAAGATGG | 10372 |
| hs132665205 | CCAGTGGAGACATTTCCTATAGG | 10373 |
| hs132665526 | AGACTTCTTTTTTAAATGAGAGG | 10374 |
| hs132665116 | ATATTTTTAATGAGATACAATGG | 10375 |
| hs132665209 | CTTTTGGCTTCAGTGTACATTGG | 10376 |
| hs132665513 | GAATTTATGCCCCAAAATGCAGG | 10377 |
| hs132665463 | GGTCGAACTGATACGTCATTAGG | 10378 |
| hs132665430 | ATATCTCCATAGCCTAAGCTTGG | 10379 |
| hs132665305 | GCTAAAACTCTCATGATCACTGG | 10380 |
| hs132665315 | TGAATTATGTTATTTGAGGTGGG | 10381 |
| hs132665096 | AGAACGTCTCTAGAAACATAAGG | 10382 |
| hs132665082 | GCAACATCACGCTGTGCTATGGG | 10383 |
| hs132665154 | TCACTTTGTGTCCCTGAGAAAGG | 10384 |
| hs132665434 | TCCTTCCCAAGCTTAGGCTATGG | 10385 |
| hs132665525 | AAAAAGTTACTTAAAAGTAAAGG | 10386 |
| hs132665191 | TGACTAATATGGGATATAGAAGG | 10387 |
| hs132665038 | ATTATTTTTTTCTTTCTAGAGGG | 10388 |
| hs132665387 | GCCTGCTTTAAAACTATACATGG | 10389 |
| hs132665110 | TTGTGTTTCAAATGAATTGAGGG | 10390 |
| hs132665256 | TGTTTTGTGTTATTCTACCCTGG | 10391 |
| hs132665278 | GTCTCTTTCTTTCACACACATGG | 10392 |
| hs132665366 | ATCCAAAGCTTACATATTGACGG | 10393 |
| hs132665422 | GGAAACTATACGTGGTTAAGAGG | 10394 |
| hs132665087 | TGTTCTTTTGAGATCTCTGTAGG | 10395 |
| hs132665075 | CATATTCCATGGTACATAGTAGG | 10396 |
| hs132665504 | GAGTGAACAAGTTTAAGAACAGG | 10397 |
| hs132665396 | TAGAAGGGAACCTCATTTGATGG | 10398 |
| hs132665451 | TGTTCCTTGAACGACAAAAACGG | 10399 |
| hs132665145 | ACATGAACCAGGTATTAAACAGG | 10400 |
| hs132665348 | AATAATACACTGAAAGGCCTTGG | 10401 |
| hs132665503 | TTATTTAGATGTATGTTGCTTGG | 10402 |
| hs132665477 | TGGCAGATACCAGGGAACACTGG | 10403 |
| hs132665363 | TTAACAGTTTCCTGAGACCAGGG | 10404 |
| hs132665374 | TTATGTCAGCATTAATTGGTTGG | 10405 |
| hs132665543 | TGATTTAGATAATCTTCTTAGGG | 10406 |
| hs132665467 | ATTAACACAGTCAACCAAACAGG | 10407 |
| hs132665163 | ACGGATACTGCAGAGCCTATTGG | 10408 |
| hs132665164 | AACCTGTCCCTAGATTTTAACGG | 10409 |
| hs132665427 | TTTTCCTTTCTATACAAAAAGGG | 10410 |
| hs132665137 | ATCATAATACTTTTTTCAAACGG | 10411 |
| hs132665536 | TGAGTTCATTGGAAGAAAATGGG | 10412 |
| hs132665292 | CTTATTCCTTCCTCAGTACTGGG | 10413 |
| hs132665056 | ACATTTTTAATTTTAAAAATGGG | 10414 |
| hs132665149 | TTTTTTGCCTGTTTAATACCTGG | 10415 |
| hs132665043 | GATCATTTCTTTCAGTCTGTGGG | 10416 |
| hs132665089 | TAAAAGTTCGAAGAGTAGTTTGG | 10417 |
| hs132665329 | GATACTATAATTATGTAATAGGG | 10418 |
| hs132665342 | ATAAAAACAGACATATGTTTAGG | 10419 |
| hs132665458 | TTAAGTAGTCATCCTAGTTATGG | 10420 |
| hs132665202 | TCTAAACTATAAACTCCTATAGG | 10421 |
| hs132665408 | AGAAACGGACAGAAATCATTTGG | 10422 |
| hs132665095 | GGTAATATAATTTAGTTAAAGGG | 10423 |
| hs132665443 | AAAGGAGCTATTATGATTTAGGG | 10424 |
| hs132665238 | TCTAATGTGCTACAACAAAATGG | 10425 |
| hs132665049 | TCTTATGTCTTATTACTTGATGG | 10426 |
| hs132665385 | ATACATGGACGTTTTCCTTATGG | 10427 |
| hs132665353 | ATAATATTGCTAACTAATAATGG | 10428 |
| hs132665173 | CGACATCTATAATAGCAAAAAGG | 10429 |
| hs132665375 | GTTCTTATGTCAGCATTAATTGG | 10430 |
| hs132665158 | CTATAAGTTGATTTTCAATATGG | 10431 |
| hs132664985 | GGAGTACAGAGGAACCACACCGG | 10432 |
| hs132664986 | AAAATGTAGCTGGAGTACAGAGG | 10433 |
| hs132664984 | GGTTACTACACTATCCGGTGTGG | 10434 |
| hs132664909 | GAACAAATGGAGATCACCAGAGG | 10435 |
| hs132664872 | AAGGAGATACCATACTCTGGAGG | 10436 |
| hs132664830 | ACATCATTTTGATATAAGAGGGG | 10437 |
| hs132664948 | ATAGACATGGAAAGGTGACAAGG | 10438 |
| hs132664960 | TGAGCTATGTGCTTTTCGCGGGG | 10439 |
| hs132664824 | ATGGGTATGAATGCGTACAGTGG | 10440 |
| hs132664974 | AGGAGACAGTCAGATCCTCATGG | 10441 |
| hs132664903 | GGTTGAGGAAGTCATCCTAAGGG | 10442 |
| hs132664973 | TCCTCATGGTAAAAGCAGCATGG | 10443 |
| hs132665001 | CATTTTTTACAGGATACATGTGG | 10444 |
| hs132664900 | GGGTGATAAGGAGGTGACAACGG | 10445 |
| hs132664927 | TATGCATGAATGTACTGTGTAGG | 10446 |
| hs132664995 | ATGGAAATAGTTAGAAACACGGG | 10447 |
| hs132664820 | ACATATTGAACAAAGAGCCAAGG | 10448 |
| hs132664970 | GCCATGCTGCTTTTACCATGAGG | 10449 |
| hs132664895 | GGGTCTGAGAGAAAGAGTAAGGG | 10450 |
| hs132664933 | CCTCTGATTCACAGGAAAAGCGG | 10451 |
| hs132665007 | ACAGCGCTAATTGATTGTGTCGG | 10452 |
| hs132664996 | CATGGAAATAGTTAGAAACACGG | 10453 |
| hs132664836 | TGCCACTTTTTAAATTACGGTGG | 10454 |
| hs132664953 | TTAATACAAAAGGCAAGGAGTGG | 10455 |
| hs132664979 | GATCCTTTCTGGACTAAAAGGGG | 10456 |
| hs132664889 | GCTTAATATTATTCATACAAGGG | 10457 |
| hs132664907 | ATATATTAAATATTGTCTGGAGG | 10458 |
| hs132664958 | ATGAGAAACTGATTAGAGGATGG | 10459 |
| hs132664812 | TCTTATCAGATATTTGTGAAGGG | 10460 |
| hs132665006 | TGTCGGTTTGCGTAAGCATGAGG | 10461 |
| hs132664920 | AAATTTCCTGCTAGGAACAGTGG | 10462 |
| hs132664981 | GGGGTAAGAACTCTGATGCTGGG | 10463 |
| hs132664868 | AGAAAGTTGGTATATATCCATGG | 10464 |
| hs132664935 | CCGCTTTTCCTGTGAATCAGAGG | 10465 |
| hs132664854 | AGTAACTTTTGTATTAGGAGAGG | 10466 |
| hs132664874 | TAAAGAATAAACAGACTCAAAGG | 10467 |
| hs132664837 | AGCCACCGTAATTTAAAAAGTGG | 10468 |
| hs132664808 | CTTTCCATACTCTATGGCACAGG | 10469 |
| hs132664940 | CTTTTGGATAAAGAATACCTTGG | 10470 |
| hs132664994 | ACACGGGTCAACAACAAGCTTGG | 10471 |
| hs132664988 | TCAAATTATCGTAATTTGAGAGG | 10472 |
| hs132664910 | ATCAAGACCAAGATACTAGCAGG | 10473 |
| hs132664888 | GTTCTGCTGAGATTAGTGATAGG | 10474 |
| hs132664886 | TTAGCTCATATTCTAATGATCGG | 10475 |
| hs132664807 | TGGCACAGGATGAAGTCAACCGG | 10476 |
| hs132664867 | TCAAACCAGTGCTAGAAAGTTGG | 10477 |
| hs132664982 | TTACCCCTTTTAGTCCAGAAAGG | 10478 |
| hs132664869 | CTTTCTAGCACTGGTTTGAGAGG | 10479 |
| hs132664972 | CATGGTAAAAGCAGCATGGCCGG | 10480 |
| hs132664991 | AGGAAAATATTTAAAACTGATGG | 10481 |
| hs132664962 | TATGAGCTATGTGCTTTTCGCGG | 10482 |
| hs132664959 | GGGAATGAGAAACTGATTAGAGG | 10483 |
| hs132664870 | ATATACCAACTTTCTAGCACTGG | 10484 |
| hs132664928 | TATATCTGAATCTCAAGAATCGG | 10485 |
| hs132664831 | CACATCATTTTGATATAAGAGGG | 10486 |
| hs132664932 | GGATTATTCCTCTGATTCACAGG | 10487 |
| hs132664921 | ATATTTTTAAATTTCCTGCTAGG | 10488 |
| hs132664862 | TGACGATTCAATCGTTTTGGTGG | 10489 |
| hs132664899 | ACTCTTTCTCTCAGACCCATTGG | 10490 |
| hs132664898 | AAGAGTAAGGGGTGATAAGGAGG | 10491 |
| hs132664947 | TGACAAGGTTGATTTAGAAAAGG | 10492 |
| hs132664913 | TAGCAGGTCCCAGAAGTCATTGG | 10493 |
| hs132664969 | TTAAGGAATCCTCATAGAAATGG | 10494 |
| hs132664952 | TTGCATACTGTAAAAAAGAATGG | 10495 |
| hs132665015 | CCTATTGATTATGCTCCAAATGG | 10496 |
| hs132664823 | ACATAATTTAGAGCATTAATGGG | 10497 |
| hs132664841 | GATAATAACACTTTTAAAAAGGG | 10498 |
| hs132664990 | TCTCAAATTACGATAATTTGAGG | 10499 |
| hs132664957 | GGATGGACTAAAACGTTTCCAGG | 10500 |
| hs132665018 | CCATTTGGAGCATAATCAATAGG | 10501 |
| hs132664832 | GCACATCATTTTGATATAAGAGG | 10502 |
| hs132664880 | TTAGAGAGCCTTATCAGAATTGG | 10503 |
| hs132664871 | TTAGTTGGTCCTCCAGAGTATGG | 10504 |
| hs132664806 | TCATCCTGTGCCATAGAGTATGG | 10505 |
| hs132664864 | ATTAAAGACGTTTAGCAAAATGG | 10506 |
| hs132664848 | CATTTTCTCTTTCTTCTAATGGG | 10507 |

What is claimed is:

1. A method of treating a subject with Duchenne muscular dystrophy (DMD), comprising:
administering to the subject a composition comprising an adeno-associated viral vector, an RNA-guided nuclease or nickase or a DNA endonuclease, and at least one dystrophin-targeted guide RNA in amount sufficient to delete a skippable exon encoding the dystrophin gene,
wherein the subject with DMD has a mutation causing a frameshift in the dystrophin gene and wherein the deletion of the skippable exon prevents the frameshift.

2. The method of claim 1, wherein the DNA endonuclease is a Cas9 endonuclease or a Cpf1 endonuclease.

3. The method of any of the preceding claims, wherein the composition comprises two dystrophin-targeted guide RNAs that recognize different DNA sequences.

4. The method of any of the preceding claims, wherein the skippable exon is selected from the group consisting of exon 2, exon 8, exon 17, exon 43, exon 44, exon 45, exon 46, exon 50, exon 51, exon 52, exon 53, and exon 55.

5. The method of any of the preceding claims, wherein the mutation is a deletion, an insertion, a duplication, or a translocation.

6. The method of claim 5, wherein the mutation is a deletion of exons 3-7, 3-19, 3-21, 4-7, 5-7, 6-7, 12-16, 18-33, 18-41, 18-44, 44, 44-47, 44-49, 44-51, 14-43, 19-43, 30-43, 35-43, 36-43, 40-43, 42-43, 45, 45-54, 12-44, 18-44, 46-47, 46-48, 46-49, 46-51, 46-53, 46-55, 21-45, 47-54, 47-56, 51, 51-53, 51-55, 45-50, 47-50, 48-50, 49-50, 50, 52, 52-63, 53, 53-55, 10-52, 45-52, 46-52, 47-52, 48-52, 49-52, 50-52, 45-54, or 48-54 of the dystrophin gene.

7. The method of any of the preceding claims, wherein the skippable exon is selected from the list in Table 1, based on the deleted exon or deleted exons that specifically occur in the dystrophin gene of the subject.

8. The method of any of the preceding claims, wherein preventing the frameshift in the dystrophin gene results in partial or complete restoration of dystrophin protein activity.

9. A method of treating a subject with Duchenne muscular dystrophy (DMD), comprising:
administering to the subject a composition comprising an adeno-associated viral vector, an RNA-guided nuclease or nickase or a DNA endonuclease, and at least one dystrophin targeted guide RNA in amount sufficient to restore the reading frame of the dystrophin gene in the subject;
wherein the dystrophin targeted guide RNA recognizes a target site in the dystrophin gene selected from the sequences identified in Table 2.

10. The method of claim 9, wherein the DNA endonuclease is a Cas9 endonuclease or a Cpf1 endonuclease.

11. The method of any one of claims 9-10, wherein the composition comprises two dystrophin-targeted guide RNAs that recognize different target sites.

12. The method of any one of claims 9-11, wherein one or more target sites recognized by the at least one dystrophin targeted guide RNA occur in an intron of the dystrophin gene.

13. The method of claims any one of 9-12, wherein restoration of the reading frame of the dystrophin gene results in partial or complete restoration of dystrophin protein activity.

14. A method of treating a subject with a genetic disease or disorder, comprising a gene editing strategy comprising:
administering to the subject a composition comprising an adeno-associated viral vector, an RNA-guided nuclease or nickase or a DNA endonuclease, at least one guide RNA, and optionally a donor DNA template in an amount sufficient to
(i) delete a skippable exon encoding a mutated gene, wherein such deletion prevents a frameshift of the mutated gene; or
(ii) correct the DNA sequence of the mutated gene via homology directed repair (HDR)
wherein the mutated gene is associated with the cause of the disease or disorder; and
wherein said method of treatment reduces at least one symptom associated with the disease or disorder.

15. The method of claim 14, wherein the DNA endonuclease is a Cas9 endonuclease or a Cpf1 endonuclease.

16. The method of any one of claims 14-15, wherein the composition comprises two guide RNAs that recognize different target sites.

17. The method of any one of claims claim 14-16, wherein the genetic disease or disorder is a muscle-related disease or disorder selected from the group consisting of Hypertrophic cardiomyopathy (HCM or CMH), amyotrophic lateral sclerosis, Becker's muscular dystrophy, central core disease, centronuclear myopathy (including myotubular myopathy), Charcot-Marie-Tooth disease, congenital muscular dystrophy, congenital myasthenic syndrome, Dejerine-Sottas disease, dermatomyositis, Duchenne muscular dystrophy, Emery-Dreifuss muscular dystrophy, facioscapulohumeral muscular dystrophy, Friedreich's ataxia, hyperthyroid myopathy, hypothyroid myopathy, inclusion body myositis, Lambert-Eaton myasthenic syndrome, Limb-girdle muscular dystrophy, mitochondrial myopathy, myasthenia gravis, myotonia congenita (including Thomsen's disease and Becker disease), nemaline myopathy, paramyotonia congenita, periodic paralysis (including hypokalemic and hyperkalemic), polymyositis, spinal and bulbar muscular atrophy, and spinal muscular atrophy.

18. The method of any one of claims 14-17, wherein the donor DNA template comprises a correct DNA sequence for insertion in the mutated gene via a homology directed repair mechanism.

19. The method of any one of claims 14-18, wherein the at least one guide RNA recognizes a target site in a gene selected from the group consisting of MYH7, TNNT2, TPM1, MYBPC3, PRKAG2, TNNI3, MYL3, TNN, MYL2, ACTC1, CSRP3, TNNC1, MYH6, VCL, MYOZ2, JPH2, PLN, CALR3, NEXN, MYPN, ACTN2, LDB3, TCAP, FLNC, SOD1, C9ORF72, and RYR.

20. The method of any one of claims 14-19, wherein the disease or disorder is suitable for treatment by an exon skipping therapeutic strategy.

21. The method of claim 20, wherein the disease or disorder is selected from the group comprising Ataxia-telangiectasia, congenital disorder of glycosylation, fronto-temporal dementia and parkinsonism linked to chromosome 17, Niemann-Pick disease type C, neurofibromatosis type 1, neurofibromatosis type 2, megalencephalic leukoencephalopathy with subcortical cysts type 1, Pelizaeus-Merzbacher disease, familial dysautonomia, Marfan syndrome, and Loeys-Dietz syndrome.

22. The method of any of the preceding claims, wherein the subject is a human.
